(19) United States
(12) Patent Application Publication
(10) Pub. No.: US 2005/0202549 A1
(43) Pub. Date: Sep. 15, 2005

(54) Crystal structure

(75) Inventors: David Graham Brown, Sandwich (GB); Colin Roger Groom, Sandwich (GB); Andrew Lee Hopkins, Sandwich (GB); Timothy Mark Jenkins, Sandwich (GB); Sarah Helen Kamp, Sandwich (GB); Margaret Mary O'Gara, Sandwich (GB); Heather Joan Ringrose, Sandwich (GB); Colin Mark Robinson, Sandwich (GB); Wendy Elaine Taylor, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/837,081

(22) Filed: Apr. 30, 2004

Related U.S. Application Data
(63) Continuation-in-part of application No. 10/427,222, filed on May 1, 2003.
(63) Continuation-in-part of application No. 10/415,839, filed on Oct. 27, 2003.

(30) Foreign Application Priority Data
May 1, 2003 (GB) 0310058.3
Oct. 24, 2002 (WO) PCT/IB02/04426
Nov. 2, 2001 (GB) 0126417.5

(51) Int. Cl. G06F 19/00; G01N 33/48; G01N 33/50; C12N 9/16

(52) U.S. Cl. 435/196; 702/19

(57) ABSTRACT

The present invention relates to the soakable crystals of a phosphodiesterase 5 (PDE5) and their uses in identifying PDE5 ligands, including PDE5 ligands and inhibitor compounds. The present invention also relates to methods of identifying such PDE5 inhibitor compounds and their medical use. The present invention additionally relates to crystals of PDE5 into which ligands may be soaked and crystals of PDE5 comprising PDE5 ligands that have been soaked into the crystal.

### RELATED APPLICATIONS AND PRIORITY CLAIMS THERETO

This application is a continuation-in-part of U.S. application Ser. No. 10/427,222 filed May 1, 2003 which is a continuation-in-part of U.S. application Ser. No. 10,415,839, filed on Apr. 30, 2003, which is the National Stage Application of International Application No. PCT/EB02/04426 filed on Oct. 24, 2002, having designated the United States. The subject matter of each of the co-pending U.S. application Ser. No. 10/415,839 and co-pending U.S. application Ser. No. 10/472,222 and International Application No. PCT/EB02/04426 is incorporated-by-reference in their entirety herein.

This application claims priority under 35 USC §119, 120 and 365 to the following patent applications: Application No. GB0126417.5 filed in the United Kingdom on Nov. 2, 2001; Application No. UK 0310058.3 filed May 1, 2003; International Application No. PCT/IB02/04426 filed in the WO on Oct. 24, 2002 and having designated the U.S. as a receiving country; U.S. application Ser. No. 10/415,839 filed on Apr. 30, 2003 and U.S. application Ser. No. 10/427,222 filed on May 1, 2003.

### TECHNICAL FIELD

The present invention relates to crystal structures of a phosphodiesterase 5 (PDE5) and ligand complexes of PDE5 and to their uses in identifying PDE5 ligands, including PDE5 inhibitor compounds. The present invention also relates to methods of identifying such PDE5 inhibitor compounds and their medical use. The present invention additionally relates to crystals of PDE5 into which ligands may be soaked and to crystals of PDE5 comprising ligands that have been soaked into the crystal. Also contemplated by the present invention is the use of crystals of PDE5 into which ligands may be soaked in identifying ligands of PDE5, including PDE5 inhibitor compounds.

### BACKGROUND OF THE INVENTION

A wide variety of biological processes, including cardiac muscle contraction, regulation of blood flow, neural transmission, glandular secretion, cell differentiation and gene expression are affected by steady state levels of the cyclic nucleotide biological second messengers cAMP and cGMP. Intracellular receptors for these molecules include cyclic nucleotide dependent protein kinases (PGK) (Lohmann et al. 1997), cyclic nucleotide-gated channels, and class I phosphodiesterases (PDEs) (Charbonneau 1990). PDEs are a large family of proteins, which were first reported by Sutherland and co-workers (Rall & Sutherland 1958, Butcher & Sutherland 1962). The family of cyclic nucleotide phosphodiesterases catalyse the hydrolysis of 3′, 5′-cyclic nucleotides to the corresponding 5′ monophosphates. Current literature shows that there are eleven related, but biochemically distinct, human phosphodiesterase gene groups and that many of these groups include more than one gene subtype giving a total of twenty genes. Some PDEs are highly specific for hydrolysis of cAMP (PDE4, PDE7, PDE8), some are highly cGMP specific (PDE5, PDE6, PDE9), and some have mixed specificity (PDE1, PDE2, PDE3, PDE10, PDE11).

All PDEs are multi-domain proteins; each PDE has a ˜270 amino acid domain located towards the C-terminus, which has a high degree of amino acid sequence conservation between families (Charbonneau 1986). This domain has been extensively studied and shown to be responsible for the common catalytic function (Francis, S. H. et al. 1994). Non-homologous segments in the remainder of the protein have regulatory function or confer specific binding properties. PDE2, PDE5, PDE6 and PDE10 are all reported to contain putative GAF domains within their regulatory amino terminal portion (Aravind & Ponting 1997 and Soderling & Beavo 2000). These GAF domains have been shown to bind cGMP but their function is not yet fully understood. Full length mammalian PDEs characterised to date are dimeric in solution, but the relevance of the dimeric structure is unknown. The structure of the regulatory segment of PDE2A bound to cGMP has recently been solved and reveals a parallel dimer of four GAF domains, with cGMP binding to only one of the two GAF domains on each monomer (Martinez, et al. 2001).

PDE5, a cGMP specific PDE, has been recognised in recent years as an important therapeutic target. It is composed of the conserved C-terminal, zinc containing, catalytic domain, which catalyses the cleavage of cGMP, and an N-terminal regulatory portion, which contains two GAF domain repeats. Each GAF domain contains a cGMP-binding site, one of high affinity and the other of lower affinity. PDE5 activity is regulated through binding of cGMP to the high and low affinity cGMP binding sites followed by phosphorylation, which occurs only when both sites are occupied (Thomas et al. 1990). PDE5 is found in varying concentrations in a number of tissues including platelets, vascular and visceral smooth muscle, and skeletal muscle. The protein is a key regulator of cGMP levels in the smooth muscle of the erectile corpus cavemosal tissue. The physiological mechanism of erection involves release of nitric oxide (NO) in the corpus cavemosum during sexual stimulation. NO then activates the enzyme guanylate cyclase, which results in increased levels of cGMP, producing smooth muscle relaxation in the corpus cavemosum and allowing in flow of blood. Inhibition of PDE5 inhibits the breakdown of cGMP allowing the levels of cGMP, and hence smooth muscle relaxation, to be maintained (Corbin & Francis 1999). Sildenafil (UK-092,480), the active ingredient of Viagra® and a potent inhibitor of PDE5, has attracted widespread attention for the effective treatment of male erectile dysfunction.

Structural information has recently been shown for the catalytic domain of PDE4b a cAMP-specific PDE (Xu et al. 2000). This structure provides information about the overall fold of the catalytic domains of the PDE family, but, to date, no structural information is known about the way in which potential inhibitors bind to the enzyme.

The X-ray structure of a recombinant PDE5 comprising the catalytic domain in complex with Sildenafil has been determined (see earlier related patent application Number PCT/IB02/04426: Example 9, “crystallisation of wild type PDE5 catalytic domain with Sildenafil”, page 44, line 29 to page 45, line 17; Example 13, “Data collection, structure determination and refinement of wild type PDE5 with Sildenafil”, page 48, line 1 to page 49, line 9; Table 4, page 91 to 248; incorporated herein by reference). An engineered form of this PDE5, PDE5*, which shows improved qualities for the production of crystals of ligand complexes, has also been produced and the structure of such a complex has been determined (see Application Number PCT/IB02/04426: Example 11, “crystallisation of PDE5* with Sildenafil”, page 46, lines 8 to 25; Example 15, “data collection, structure determination and refinement of PDE5* with Sildenafil”, page 50, line 9 to page 51, line 6; and Table 6, “atomic co-ordinates for baculovirus-expressed PDE5* complex with Sildenafil”, pages 328 to 408; incorporated herein by reference). These complexes not only provide important structural information about this novel family of proteins but also assist the design of more potent and specific inhibitors of PDEs to treat the many diseases where PDEs play a role.

It is desirable to be able to obtain crystals of biological macromolecules in a complex with their ligands in order to obtain details of specific ligand interactions at a level of molecular resolution and, preferably, at atomic resolution. This invariably forms the critical step in providing information from which further, and possibly improved, ligands of the macromolecule may be designed and subsequently tested in further rounds of crystallisation. It is therefore most desirable that this process of obtaining complexes of the macromolecule be rapid and reproducible and minimise the need to constantly find new crystallisation conditions for each potential complex or use involved and time intensive structure solution methods for the solution of each potential complex structure. To this end the most desirable crystal form of a macromolecule of interest is one which is amenable to having potential ligands soaked into the preformed crystal of the apo macromolecule. This has distinct advantages over conventionally produced co-crystals. In particular no new crystallisation conditions need to be found for each potential complex. The lattice symmetry and cell dimensions for each new ligand complex will be substantially the same as for the apo macromolecule such that the data collection parameters for the apo and all complex crystals will essentially be identical. The structure solution can then occur using rapid difference fourier methods, thus avoiding more involved time and labour intensive phasing methods. Consequently, soakable crystals of macromolecules allow for particularly rapid screening and structural determination of new macromolecule-ligand complexes.

The engineered form of PDE5, PDE5*, allows the production of soakable crystals of ligand complexes, and the structure of such a soaked PDE5*-ligand complex has been determined.

### SUMMARY OF THE INVENTION

It has been found that PDE5 can be crystallised. It has also been found that manipulating the wild-type PDE5 amino acid sequence can facilitate the crystallisation of PDE5. Specifically, it has been found that manipulations of certain portions of the PDE5 amino acid sequence can facilitate the crystallisation of PDE5. More specifically it has been shown that particular manipulations of the PDE5 amino acid sequence can provide soakable crystal forms of PDE5 into which potential PDE5 ligands may be introduced by the process of crystal soaking. Manipulating the wild-type PDE5 amino acid sequence results in a sequence modified PDE5 protein.

It has been shown that manipulations of the catalytic domain of PDE5, specifically the 657-682 region of PDE5 (the “loop region”), can facilitate the crystallisation of PDE5 and particularly provide soakable crystals of PDE5. More specifically, manipulations of the loop region amino acid sequence (HRGVNNSYIQRSEHPLAQLYCHSIME=SEQ ID NO: 1) of PDE5 can facilitate the crystallisation of PDE5 and provide soakable crystals of PDE5. This manipulation can be achieved by deletion, addition or substitution of one or more amino acid residues of the PDE5 loop region or it can be achieved by complete replacement of the PDE5 loop region with a loop region (or other amino acid sequence) from another protein, preferably another PDE, more preferably PDE4, most preferably PDE4b (Genbank Accession Number=L20966).

Crystals of PDE5 have been found to be useful for screening for PDE5 ligands, especially PDE5 inhibitors by (i) co-crystallising PDE5 with the PDE5 ligand (e.g. PDE5 inhibitor), as shown in Application Number PCT/IB02/04426 (Example 9, “crystallisation of wild type PDE5 catalytic domain with Sildenafil”, page 44, line 29 to page 45, line 17; Example 11, “crystallisation of PDE5* with Sildenafil”, page 46, lines 8 to 25; Example 13, “Data collection, structure determination and refinement of wild type PDE5 with Sildenafil”, page 48, line 1 to page 49, line 9; Example 15, “data collection, structure determination and refinement of PDE5* with Sildenafil”, page 50, line 9 to page 51, line 6; Table 4 “atomic co-ordinates for wild type PDE5 complexed with Sildenafil”, pages 91 to 240; and Table 6, “atomic co-ordinates for baculovirus-expressed PDE5* complex with Sildenafil”, pages 328 to 408; incorporated herein by reference), or (ii) by soaking the PDE5 ligand (e.g. PDE5 inhibitor) into the crystal of PDE5, as shown in the present invention.

PDE5 ligands, especially PDE5 inhibitors, as identified by the methods of the present invention are useful in curative, palliative or prophylactic treatments.

Thus, the present invention provides the following (numbered) aspects:

Aspects

1. A crystal of phosphodiesterase 5 (PDE5), wherein the crystal is soakable.
2. The crystal of PDE5 according to aspect 1, wherein said PDE5 is from a mammal.
3. The crystal of PDE5 according to aspect 1 or aspect 2, wherein said PDE5 is from a human.
4. The crystal of PDE5 according to any one of aspects 1 to 3, wherein said PDE5 is an isoform selected from the group consisting of PDE5A1, PDE5A2, PDE5A3 and PDE5A4.
5. The crystal of PDE5 according to any one of aspects 1 to 4, wherein said PDE5 comprises SEQ ID NO: 1 (PDE5 loop region) or a homologue, fragment, variant, analogue or derivative thereof.

SEQ ID NO: 1 is the so-called “loop region” of PDE5. This loop region or a homologue, fragment, variant, analogue or derivative thereof includes additions, deletions or substitutions of amino acid residues comprised within the loop region.

Preferably, a variant in relation to the amino acid sequence of the crystal of the PDE5 of the present invention includes the deletion or substitution of the histidine (His/H) residue as shown emboldened and underlined in SEQ ID NO: 1 (HRGVNNSYIQRSEHPLAQLYCHSIME). Replacement of said histidine (H) residue is preferably by way of incorporating one or more amino acid residues (other than histidine), preferably wherein said amino acid residues are neutral or non-polar.

More preferably, a variant in relation to the amino acid sequence of the crystal of the PDE5 of the present invention includes the complete replacement of the loop region with a loop region (or other amino acid sequence) from another protein, preferably a PDE, more preferably PDE4, most preferably PDE4b (see hereinafter).
6. The crystal of PDE5 according to any one of aspects 1 to 5, wherein said PDE5 comprises SEQ ID NO: 4 (PDE4 loop) or a homologue, fragment, variant, analogue or derivative thereof.
7. The crystal of PDE5 according to any one of aspects 1 to 6, wherein said PDE5 comprises SEQ ID NO: 5 (PDE5*=loop swapped PDE5 catalytic domain) or a homologue, fragment, variant, analogue or derivative thereof
8. The crystal of PDE5 according to any one of aspects 1 to 7, wherein said PDE5 comprises SEQ ID NO: 6 (full PDE5 sequence comprising PDE5*) or a homologue, fragment, variant, analogue or derivative thereof.
9. The crystal of PDE5 according to any of the preceding aspects which is grown using polyethylene glycol as a precipitant. Polyethylene glycol of molecular weights 2000 to 8000 are commonly used. Preferably the molecular weight of the polyethylene glycol is 4000. The polyethylene glycol may be at a concentration of between 10% and 30%, but is most preferably 20%. Additionally, the PDE5 protein used to grow the crystals is desired to be of a concentration between 1 and 20 mg/ml, preferably between 5 and 15 mg/ml and most preferably is at 10 mg/ml concentration.
10. The crystal of PDE5 according to any of the preceding aspects which is grown in a buffer in the pH range of 6.5 to 8.0. Preferably the pH is in the range of 7.0 to 7.8; most preferably the pH is 7.4. The buffer should be one capable of providing buffer capacity over the required pH range, preferably HEPES.
11. The crystal of PDE5 according to any of the preceding aspects which is grown in the presence of an alcohol. Preferably the alcohol is isopropanol.
12. The crystal of PDE5 according to any of the preceding aspects which is grown in a solution containing HEPES buffer, polyethylene glycol 4000 and iso-propanol. Preferably the solution contains 0.1M sodium HEPES pH 7.4, 20% polyethylene glycol 4000 and 10% iso-propanol. The crystal is grown from solution incubated at or below room temperature (20-25° C.). Preferably the crystal is grown from a solution incubated at a temperature within the range of 2-6° C. More preferably, the crystal is grown from a solution incubated at about 4° C.

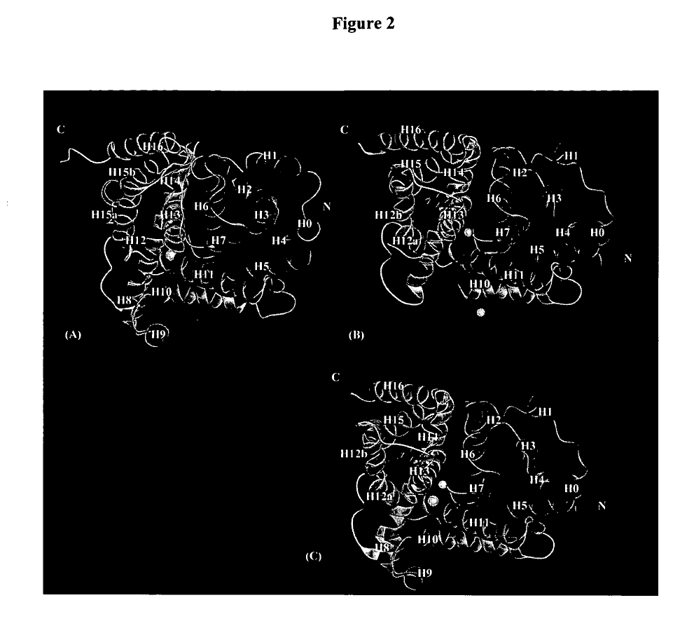

Soakable crystals may be grown using a variety of methods such as dialysis, sitting drop vapour diffusion or batch methods, microcrystallisation methods, micro or macro seeding methods or gel crystallisation methods but are preferably grown using hanging drop vapour diffusion.
13. The crystal of PDE5 as defined in any of the preceding aspects, which has one or more of the following characteristics:
   (a) a space group C2;
   (b) unit cell dimensions a˜56 Å±1%, b˜77 Å±1%, c˜81 Å±1%, α=γ=90°, β=103°≅1%;
   (c) 1 molecule per asymmetric unit;
   (d) comprises a PDE5 of a molecular weight of approximately 40 kDa±2 kDa;
   (e) a calculated solvent content of approximately 44±5%; and
   (f) a monoclinic crystal system.
14. The crystal of PDE5 according to any of the preceding aspects, wherein PDE5 has an active site within the third sub-domain of the protein which is bounded by Helices 15 (H15 813-824) and 14 (H14 772-797), the C-terminus of Helix 13 (H13 749-765), and the C-terminus of Helix 11 (H11 706-721) along with the loop region between Helices 11 and 12a (H12a 725-731) as shown in FIG. 2.
15. The crystal of PDE5 according to aspect 14, wherein the active site is capable of accommodating a pyrazolo-pyrimidinone. An example of a preferred pyrazolo-pyrimidinone is UK-088,800.

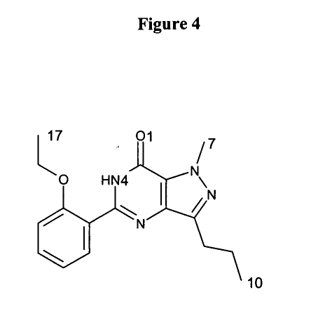

UK-088,800 is illustrated in FIG. 4 and is a structural representation of the compound with the chemical name 5-(2-ethoxyphenyl)-1-methyl-3-propyl-1,6-dihydro-7H-pyrazolo[4,3-d]pyrimidin-7-one.
16. The crystal of PDE5 according to any of the preceding aspects, wherein PDE5 has an active site within the third sub-domain of the protein and comprises Leu 765, Ala 767 and Ile 768 and one or more of Phe 820, Val 782, Phe 786, Tyr 612, Leu 804, Ala 779, Ala 783, Ile 813, Met 816 and Gln 817.
17. The crystal of PDE5 according to aspect 16, wherein the active site is capable of accommodating a pyrazolo-pyrimidinone. An example of a preferred pyrazolo-pyrimidinone is UK-088,800.
18. The crystal of PDE5 according to any of the preceding aspects, wherein said PDE5 has a three-dimensional structure characterised by the atomic co-ordinates set out in Table 4 (PDE5* apo C2 form) or a derivative set as expressed in any reference frame.
19. A heavy atom derivative of the crystal of PDE5 according to any of the preceding aspects.
20. The crystal of PDE5 according to any of the preceding aspects, wherein a PDE5 ligand has been soaked in. The process of soaking may involve transferring the crystal to be soaked from the solution in which it is grown into a stabilising solution containing the ligand or to which the ligand is to be added once the crystal is present in it. It is desirable that the stabilising solution has the physical properties such that the crystal, when transferred, retains its structural integrity and does not crack or dissolve or alter significantly in its crystal parameters, symmetry or cell dimensions. Preferably, the stabilising solution comprises some or all of the chemical constituents from which the crystal was grown. More preferably, the stabilising solution is of similar or identical composition to the solutions used for crystal growth as set out in aspects 9 to 12 above, although the concentration of precipitant used may be slightly increased. Most preferably the stabilising solution has the same pH as the crystal growth solution. The ligand may be added to the solution containing, or to contain, the crystal for soaking as a solid or in a liquid form (i.e. the ligand already being in solution). Preferably the ligand is in solution and the solvent maybe aqueous, organic or non-organic. Most preferably the ligand is in DMSO. The ligand is commonly added to the soaking solution to yield a final ligand concentration above the expected binding constant for the ligand by PDE5; preferably this is greater than or equal to 10 times the binding constant. The final concentration of ligand present in the crystal soaking solution is preferably between 0.1 and 20 mg/ml, more preferably between 0.5 and 10 mg/ml, and most preferably between 1 and 5 mg/ml.
21. The crystal of PDE5 according to aspect 20, wherein said PDE5 ligand is a PDE5 inhibitor.
22. The crystal of PDE5 according to aspect 21, wherein said PDE5 inhibitor is a pyrazolo-pyrimidinone, preferably UK-088,800.
23. The crystal of PDE5 according to aspect 22, wherein said PDE5 has a three-dimensional structure characterised by the atomic co-ordinates set out in Table 5 (PDE5*-UK-088,800 complex) or a derivative set as expressed in any reference frame.
24. Use of the atomic co-ordinates determined from the crystal of PDE5 according to any one of the preceding aspects for deriving a three-dimensional structure of a PDE5 or a mutant, derivative, variant, analogue, homologue, sub-domain or fragment thereof.
25. Use according to aspect 24, wherein the PDE5 sub-domain is the catalytic domain.
26. Use of the three-dimensional structure of PDE5 as derivable according to aspect 24 or aspect 25 to computationally or otherwise evaluate the binding interactions of a chemical compound with an active site on PDE5.
27. Use of the three-dimensional structure of PDE5 as derivable according to aspect 26 to design a compound capable of associating with PDE5. Preferably said compound is a pyrazolo-pyrimidinone, more preferably UK-088,800.
28. Use according to aspect 26 or aspect 27, wherein the compound is a PDE5 ligand.
29. Use according to aspect 28, wherein said compound is a PDE5 inhibitor.
30. Use according to aspect 29, wherein said PDE5 inhibitor is a pyrazolo-pyrimidinone, preferably UK-088,800.
31. A method of selecting a compound capable of associating with PDE5 from a group of potential PDE5 ligand compounds comprising the following steps:
   i. soaking the crystal of PDE5 according to any one of aspects 1 to 19 in a solution containing a potential PDE5 ligand compound;
   ii. determining the three-dimensional structure from the soaked crystal; and
   iii. assessing whether the compound is bound to PDE5.
32. A compound selected by the use according to aspect 31.
33. A compound designed by the use according to any one of aspects 27 to 30 or identified by the method of aspect 31.
34. The compound according to aspect 33, which is a PDE5 inhibitor.
35. A method of selecting a PDE5 ligand from a group of potential PDE5 ligands, comprising the following steps:
   (a) computationally creating a three-dimensional representation of the structure of PDE5 as derivable according to aspect 24 or aspect 25, and a three-dimensional representation of the structure of the potential PDE5 ligand;
   (b) co-displaying the three-dimensional representation of the potential PDE5 ligand with the three-dimensional representation of the PDE5 structure; and
   (c) assessing whether the three-dimensional representation of the potential PDE5 ligand fits the three-dimensional representation of an active site of the PDE5 structure.

Preferably, ligands are built using molecular graphics tools, more preferably the designed ligand is energy minimised prior to co-display and analysis.
36. The method according to aspect 35, further comprising the following steps:
   (d) incorporating the potential PDE5 ligand in a biological PDE5 activity assay; and
   (e) determining whether the potential PDE5 ligand modulates PDE5 activity in said assay.
37. The method according to aspect 35 or aspect 36 wherein said potential PDE5 ligand is a potential PDE5 inhibitor compound and said potential PDE5 inhibitor compound inhibits PDE5 activity.
38. A PDE5 ligand selected by the method of any one of aspects 35 to 37. Preferably the ligand is a PDE5 inhibitor.
39. Use of a PDE5 ligand according to aspect 38 as a pharmaceutical. Preferably the ligand is a PDE5 inhibitor.
40. A pharmaceutical composition comprising one or more PDE5 ligands according to aspect 38 and one or more pharmaceutically acceptable excipients.
41. Use of a PDE5 ligand according to aspect 38 in the manufacture of a medicament for the prophylaxis or treatment of a condition, disease, disorder or dysfunction where the inhibition of PDE5 is prophylactically or therapeutically beneficial.
42. Use according to aspect 41, wherein said disorder is a mammalian sexual disorder.

The curative, palliative or prophylactic treatments contemplated by the present invention include the curative, palliative or prophylactic treatment of mammalian sexual disorders, in particular the treatment of mammalian sexual dysfunctions such as male erectile dysfunction (MED), impotence, female sexual dysfunction (FSD), clitoral dysfunction, female hypoactive sexual desire disorder, female sexual arousal disorder (FSAD), female sexual pain disorder or female sexual orgasmic dysfunction (FSOD) as well as sexual dysfunction due to spinal cord injury or selective serotonin re-uptake inhibitor (SSRI) induced sexual dysfunction but, clearly, will also be useful for treating other medical conditions for which PDE5 inhibitor is indicated. Such conditions include premature labour, dysmenorrhoea, benign prostatic hyperplasia (BPH), bladder outlet obstruction, incontinence, stable, unstable and variant (Prinzmetal) angina, hypertension, pulmonary hypertension, chronic obstructive pulmonary disease, coronary artery disease, congestive heart failure, atherosclerosis, conditions of reduced blood vessel patency, e.g. post-percutaneous transluminal coronary angioplasty (post-PTCA), peripheral vascular disease, stroke, nitrate induced tolerance, bronchitis, allergic asthma, chronic asthma, allergic rhinitis, diseases and conditions of the eye such as glaucoma, optic neuropathy, macular degeneration, elevated intra-occular pressure, retinal or arterial occlusion and diseases characterised by disorders of gut motility, e.g. irritable bowel syndrome (IBS).

Further medical conditions for which a PDE5 inhibitor is indicated, and for which treatment with compounds of the present invention may be useful include pre-eclampsia, Kawasaki's syndrome, nitrate tolerance, multiple sclerosis, diabetic nephropathy, neuropathy including autonomic and peripheral neuropathy and in particular diabetic neuropathy and symptoms thereof e.g. gastroparesis, peripheral diabetic neuropathy, Alzheimer's disease, acute respiratory failure, psoriasis, skin necrosis, cancer, metastasis, baldness, nutcracker oesophagus, anal fissure, haemorrhoids, the insulin resistance syndrome, diabetes, hypoxic vasoconstriction as well as the stabilisation of blood pressure during haemodialysis.

Particularly preferred conditions include MED and FSD (preferably FSAD).

Further (numbered) aspects of the present invention include:
43. Use of the atomic co-ordinates determined from the crystal of PDE5 according to any one of aspects 1 to 23, to solve the crystal structure of a mutant, derivative, fragment, variant, analogue, homologue or complex of PDE5.
44. Use of the atomic co-ordinates determined from the crystal of PDE5 according to any one of aspects 1 to 23, to produce a model of the three-dimensional structure of PDE5-related proteins.
45. Use of the three-dimensional structure of PDE5 as derivable according to aspect 24 or aspect 25 to design site-directed mutants that mimic other PDE5 isoforms or variants thereof.
46. A method of soaking a chemical compound into a crystal according to any one of aspects 1 to 19 comprising the following steps:
   a) incubating the crystal in an aqueous stabilising solution comprising buffer and polyethylene glycol;
   b) combining the chemical compound with the stabilising solution preferably wherein the chemical compound is in solid or liquid form, more preferably in liquid form, most preferably solublised in DMSO; and
   c) optionally adding a cryo-protectant to the stabilising solution.
47. A method according to aspect 46, wherein the stabilising solution comprises polyethylene glycol which is of the molecular weight 4000. The polyethylene glycol may be at a concentration of between 10% and 30%, but is most preferably 20%. Additionally the pH is preferably in the range of 7.0 to 7.8; most preferably the pH is 7.4. The buffer should be one capable of providing buffer capacity over the required pH range; preferably HEPES. Preferably the stabilising solution contains an alcohol; most preferably this is isopropanol. Preferably the stabilising solution contains 0.1M sodium HEPES pH 7.4, 20% polyethylene glycol 4000 and 10% iso-propanol. Examples of suitable cryoprotectants include 2-methyl-2,4-pentanediol (MPD) and organic polymers e.g. lower molecular weight PEG. Carbohydrates such as sorbitol or xylitol, and alcohols may also be used. Preferably the cryoprotectant is glycerol.

A PDE5 ligand (also known as a PDE5 inhibitor compound) according to aspect 38 (hereinafter referred to as “the compound”) can be administered alone but, in human therapy, will generally be administered in admixture with a suitable pharmaceutical excipient diluent or carrier selected with regard to the intended route of administration and standard pharmaceutical practice. Thus, the pharmaceutical compositions, pharmaceuticals and medicaments contemplated by the present invention may be formulated in various ways well-known to one of skill in the art and administered by similarly well-known methods.

For example, the compound of the invention can be administered orally, buccally or sublingually in the form of tablets, capsules (including soft gel capsules), ovules, elixirs, solutions or suspensions, which may contain flavouring or colouring agents, for immediate-, delayed-, modified-, or controlled-release such as sustained-, dual-, or pulsatile delivery applications. The compound may also be administered via intracavernosal injection. The compound may also be administered via fast dispersing or fast dissolving dosages forms or in the form of a high-energy dispersion or as coated particles. Suitable pharmaceutical formulations of the compound may be in coated or un-coated form as desired.

Such tablets may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compound may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate-modifying excipients maybe present both within the dosage form i.e. within the matrix, and/or on the dosage form i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl flimarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compound can also be administered parenterally, for example, intracavernosally, intravenously, intra-arterially, intraperitoneally, intrathecally, intraventricularly, intraurethrally intrastemally, intracranially, intramuscularly or subcutaneously, or they may be administered by infusion or needleless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

For oral and parenteral administration to human patients, the daily dosage level of the compound will usually be from 10 to 500 mg (in single or divided doses).

Thus, for example, tablets or capsules of the compound may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions (including MED and FSD), the compound may be taken as a single dose on an “as required” basis (i.e. as needed or desired).

The compound can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™ or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

The compound may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the compound can be administered in the form of a suppository or pessary, or they may be applied topically in the form of a gel, hydrogel, lotion, solution, cream, ointment or dusting powder. The compound may also be dermally administered. The compound may also be transdermally administered, for example, by the use of a skin patch. The compound may also be administered by the ocular, pulmonary or rectal routes.

For ophthalmic use, the compound can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, the compound may be formulated in an ointment such as petrolatum.

For application topically to the skin, the compound of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compound may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

Generally, in humans, oral administration the compound is the preferred route, being the most convenient and, for example in MED, avoiding the well-known disadvantages associated with intracavernosal (i.c.) administration. A preferred oral dosing regimen in MED for a typical man is from 25 to 250 mg of compound when required. In circumstances where the recipient suffers from a swallowing disorder or from impairment of drug absorption after oral administration, the drug may be administered parenterally, sublingually or buccally.

For veterinary use, the compound, or a veterinarily acceptable salt thereof, or a veterinarily acceptable solvate or pro-drug thereof, is administered as a suitably acceptable formulation in accordance with normal veterinary practice and the veterinary surgeon will determine the dosing regimen and route of administration which will be most appropriate for a particular animal.

The present invention provides the following numbered preferred aspects:

Preferred Aspects of the Invention

1. A crystal of sequence-modified PDE5 protein comprising SEQ ID NO: 4 into which a compound of the formula represented in FIG. 4 is capable of being soaked such that the compound is bound to the active site of the protein.

The term “sequence modified PDE5 protein” as used herein includes the wild-type PDE5 protein amino acid sequence which has been manipulated to facilitate the crystallisation of the protein, and in particular provide soakable crystal forms of the protein into which a PDE5 ligand or potential ligand may be introduced by soaking the crystal in a solution comprising the ligand so that the ligand can enter the crystal and bind to the active site of the protein in the process of crystal soaking. The term includes a PDE5 protein that has been subjected to manipulations of the amino acid sequence of the catalytic domain of PDE5, specifically the 657-682 region of PDE5 (the “loop region”) or SEQ ID No:1, and that facilitate the crystallisation of the protein and particularly provide soakable crystal forms of the protein. The relevant manipulations include manipulation by deletion, addition or substitution of one or more amino acid residues of the PDE5 loop region. Such manipulations for example include the deletion or substitution of the histidine (His/H) residue as shown emboldened and underlined in SEQ ID NO: 1 (HRGVNNSYIQRSEHPLAQLYCHSIME) to incorporate one or more amino acid residues (other than histidine), particularly amino acid residues that are neutral or non-polar. The relevant manipulations also include manipulation by complete replacement of the PDE5 loop region with a loop region (or other amino acid sequence) from another protein or protein sequence, for example another PDE such as PDE4, preferably PDE4b. The sequence modified PDE5 protein may comprise a manipulated sequence of the full PDE5 sequence or alternatively a manipulated sequence of a sub-domain or fragment of the PDE5 sequence, and is preferably a manipulated sequence of the PDE5 catalytic domain.
2. A crystal according to preferred aspect 1 comprising SEQ ID NO:5.
3. A crystal according to either preferred aspect 1 or preferred aspect 2 which is grown using polyethylene glycol as a precipitant Polyethylene glycol of molecular weights 2000 to 8000 are commonly used. Preferably the molecular weight of the polyethylene glycol is 4000. The polyethylene glycol may be at a concentration of between 10% and 30%, but is most preferably 20%. Additionally, the protein used to grow the crystals is desired to be of a concentration between 1 and 20 mg/ml, preferably between 5 and 15 mg/ml and most preferably is at 10 mg/ml concentration.
4. A crystal according to any of the preceding preferred aspects which is grown in a buffer in the pH range of 6.5 to 8.0. Preferably the pH is in the range of 7.0 to 7.8; most preferably the pH is 7.4. The buffer should be one capable of providing buffer capacity over the required pH range, preferably HEPES.
5. A crystal according to any of the preceding preferred aspects which is grown in the presence of an alcohol. Preferably the alcohol is isopropanol.
6. A crystal according to any of the preceding preferred aspects which is grown in a solution containing HEPES buffer, polyethylene glycol 4000 and iso-propanol. Preferably the solution contains 0.1M sodium HEPES pH 7.4, 20% polyethylene glycol 4000 and 10% iso-propanol. The crystal is grown from a solution incubated at or below room temperature (20-25° C.). Preferably the crystal is grown from a solution incubated at a temperature within the range of 2-6° C. More preferably, the crystal is grown from a solution incubated at about 4° C.
7. A crystal according to any of the preceding preferred aspects, which has one or more of the following characteristics:
   (a) a space group C2;
   (b) unit cell dimensions a˜56 Å±1%, b˜77 Å±1%, c˜81 Å±1%, α=γ=90°, β=103°±1%;
   (c) 1 molecule per asymmetric unit;
   (d) comprises a protein of a molecular weight of approximately 40 kDa±2 kDa;
   (e) a calculated solvent content of approximately 44±5%; and
   (f) a monoclinic crystal system.
8. A crystal according to any of the preceding preferred aspects, wherein the protein has an active site within the third sub-domain of the protein which is bounded by Helices 15 (H15 813-824) and 14 (H14 772-797), the C-terminus of Helix 13 (H13 749-765), and the C-terminus of Helix 11 (H11 706-721) along with the loop region between Helices 11 and 12a (H12a 725-731) as shown in FIG. 2.
9. A crystal according to any of the preceding preferred aspects, wherein the protein has an active site within the third sub-domain of the protein and comprises Leu 765, Ala 767 and Ile 768 and one or more of Phe 820, Val 782, Phe 786, Tyr 612, Leu 804, Ala 779, Ala 783, Ile 813, Met 816 and Gln 817.
10. A crystal according to in any of the preceding preferred aspects, wherein the protein has a three-dimensional structure characterised by the atomic co-ordinates set out in Table 4 or a derivative set as expressed in any reference frame.
11. A crystal according to any of the preceding preferred aspects, wherein a PDE5 ligand has been soaked in. The process of soaking may involve transferring the crystal to be soaked from the solution in which it is grown into a stabilising solution containing the ligand or to which the ligand is to be added once the crystal is present in it so that the ligand can enter the crystal and preferably bind to the active site of the protein. It is desirable that the stabilising solution has the physical properties such that the crystal, when transferred, retains its structural integrity and does not crack or dissolve or alter significantly in its crystal parameters, symmetry or cell dimensions. Preferably, the stabilising solution comprises some or all of the chemical constituents from which the crystal was grown. More preferably, the stabilising solution is of similar or identical composition to the solutions used for crystal growth as set out in aspects 3 to 6 above, although the concentration of precipitant used may be slightly increased by between 1 to 10%. Most preferably the stabilising solution has the same pH as the crystal growth solution. The ligand may be added to the solution containing, or to contain, the crystal for soaking as a solid or in a liquid form (i.e. the ligand already being in solution). Preferably the ligand is in solution and the solvent maybe aqueous, organic or non-organic. Most preferably the ligand is in DMSO. The ligand is commonly added to the soaking solution to yield a final ligand concentration above the expected binding constant for the ligand by PDE5; preferably this is greater than or equal to 10 times the binding constant. The final concentration of ligand present in the crystal soaking solution is preferably between 0.1 and 20 mg/ml, more preferably between 0.5 and 10 mg/ml, and most preferably between 1 and 5 mg/ml. Optionally a cryoprotectant such as 2-methyl-2,4-pentanediol (MPD), alcohols and organic polymers e.g. lower molecular weight PEG or carbohydrates such as sorbitol or xylitol, may also added to the soaking solution if the crystal is to be frozen prior to data collection.
12. A crystal according to preferred aspect 11, wherein the protein has a three-dimensional structure characterised by the atomic co-ordinates set out in Table 5 or a derivative set as expressed in any reference frame.
13. A heavy atom derivative of a crystal according to any of the preceding preferred aspects.
14. Use of the atomic co-ordinates determined from a crystal according to any one of preferred aspects 1 to 13 for deriving a three-dimensional structure of a PDE5 or a mutant, derivative, variant, analogue, homologue, sub-domain or fragment thereof. The co-ordinate set or a part thereof can be used according to known methods to provide a full or partial phasing model for molecular replacement procedures to determine the X-ray crystal structure of a structurally similar or related protein or protein sub-domains or fragments, additionally the coordinate set can provide a structural model from which NMR assignments for NMR structure solution of structurally similar or related proteins or protein sub-domains or fragments can be determined.
15. Use according to preferred aspect 14, wherein the sub-domain is the catalytic domain.
16. A method of identifying a compound which is a ligand of PDE5 comprising the following steps:
   a) providing a crystal according any one of preferred aspects 1 to 13,
   b) contacting the crystal with the compound under conditions conducive to soaking the compound into the crystal,
   c) determining, by X-ray diffraction and structure solution, whether or not the compound is bound to the active site of the protein in the resultant soaked crystal,
   d) optionally assaying the ligand to determine whether it is an inhibitor of PDE5.
17. The crystal may be soaked under the conditions as set out in preferred aspect 11 and the X-ray crystal structure may be determined by X-ray diffraction of the crystal and application of any suitable known method such as direct methods, heavy atom phasing methods, single or multiple anomalous dispersion, molecular replacement or difference Fourier methods. The presence or absence of the ligand at the active site of the protein can be determined from the electron density maps, for example from difference Fourier maps or omit maps, calculated from the X-ray diffraction data.
18. A method according to preferred aspect 17 wherein the compound is based on the structural template as shown in FIG. 4.
19. A method of designing a compound capable of associating with PDE5, comprising the following steps:
   (a) computationally creating a three-dimensional representation of the structure of the protein present in the crystal according to any of preferred aspects 1 to 13, or a fragment or subdomain thereof,
   (b) computationally creating a three-dimensional representation of the structure of a compound,
   (c) co-displaying the three-dimensional representation of the compound structure with the three-dimensional representation of the protein structure,
   (d) assessing whether the three-dimensional representation of the compound structure fits the three-dimensional representation of an active site of the protein structure,
   (e) optionally making assaying the ligand to determine whether it is an inhibitor of PDE5.
20. A method of selecting a PDE5 ligand from a group of potential PDE5 ligands, comprising the following steps:
   (a) computationally creating a three-dimensional representation of the structure of the protein present in the crystal according to any of preferred aspects 1 to 13, or a fragment or subdomain thereof,
   (b) computationally creating a three-dimensional representation of the structure of the ligand,
   (c) co-displaying the three-dimensional representation of the ligand structure with the three-dimensional representation of the protein structure,
   (d) assessing whether the three-dimensional representation of the ligand structure fits the three-dimensional representation of an active site of the protein structure,
   (e) optionally making and assaying the ligand to determine whether it is an inhibitor of PDE5.
21. According to preferred embodiments of the preferred aspects 19 and 20 the three-dimensional representation of the structure of the protein may be directly derived from the atomic coordinates determined for the crystal and may be suitably displayed to identify the active site of the protein as defined in preferred aspect 8 and 9. The active site of the protein may be computationally and graphically rendered to display the atomic radii of the atoms comprising the active site such that a similarly graphically rendered compound molecule may be docked into the active site to look for the goodness of fit between the protein active site and the compound, preferably by examination for steric clashes and/or favourable packing and/or bonding interactions between the groups and atoms comprising the active site of the protein and those of the compound. Alternatively the resulting docked compound-protein model may be analysed computationally to assess the fit or may be adjusted computationally, for example by molecular dynamics and energy minimisation of the model, to improve the fit of the compound in the active site prior to fit analysis.
22. Use of the three-dimensional structure as derivable according to preferred aspect 17 to design site-directed mutants of PDE5.

In a preferred embodiment of the preferred aspect of the invention a site directed mutant is preferably designed by computationally creating a three-dimensional representation of the structure of the protein present in the crystal according to any of preferred aspects 1 to 14, or a protein fragment or protein subdomain as may be directly derived from the atomic coordinates determined for the crystal. The model may be overlayed with, or least squares fitted against, a model of a similar or related protein or protein sequence fragment in order to allow substitution of amino acids or whole peptide regions between the model on a knowledge based basis to alter the physical characteristics of the protein, for example to substitute a known unstable loop or region with a known stable loop or region between structures (the stability of a region of protein sequence can be judged by the B factors for the protein region in the coordinate set). One or more side chain replacements, additions or deletions may be made in the model of the protein or whole fragments of polypeptide may be replaced, added or deleted to potentially affect the physical property of the protein, for example, ability to crystallise by alteration of unstable regions, solubility by substitution of hydrophobic groups with hydrophilic groups. Optionally the site directed mutant of the protein may be made and tested for the improvement in the physical parameter for example by assessing ability to crystallise the mutated protein or the solubility of the mutated protein in comparison to the native protein. Alternatively site directed mutation of the active site residues may be designed in order to assess side chains involved in key bond donor or acceptor functions with a PDE5 ligand or potential ligand compound, or to design active site mutations to better accommodate a compound. This may be achieved by co-displaying the protein and compound models as detailed in preferred aspects 18 and/or 19 and making replacements of protein side chains at the active site of the protein model in order to assess, either by graphics visualisation of the fit and bonding interactions between the compound and the protein model or by computational analysis of the model complex for steric fit or clashing and/or bonding interactions, whether a compound is better accommodated in the active site by such changes; this process may optionally involve model energy minimisation and/or molecular dynamics steps as described in the persevered embodiments in preferred aspects 18 and/or 19. Optionally the site directed mutant of the protein may be made and assayed in a PDE5 ligand binding assay.
23. A method of soaking a compound into a crystal according to any one of preferred aspects 1 to 13 comprising the following steps:
   (a) incubating the crystal in an aqueous stabilising solution comprising buffer and polyethylene glycol;
   (b) combining the compound with the stabilising solution; and
   (c) optionally adding a cryo-protectant to the stabilising solution.

Preferably the stabilising solution is of similar or identical composition to the solutions used for crystal growth as set out in aspects 3 to 6 above, although the concentration of precipitant used may be slightly increased by between 1 to 10%. Most preferably the stabilising solution has the same pH as the crystal growth solution. More preferably the stabilising solution comprises 0.1M sodium hepes pH 7.4, 20% PEG 4000, 10% isopropanol. Preferably the compound is in solution and the solvent maybe aqueous, organic or non-organic. Most preferably the compound is in DMSO. The ligand is commonly added to the soaking solution to yield a final ligand concentration above the expected binding constant for the ligand by PDE5; preferably this is greater than or equal to 10 times the binding constant. The final concentration of ligand present in the crystal soaking solution is preferably between 0.1 and 20 mg/ml, more preferably between 0.5 and 10 mg/ml, and most preferably between 1 and 5 mg/ml. Preferably the cryoprotectant is selected from one or more of 2-methyl-2,4-pentanediol (MPD), alcohols, organic polymers e.g. lower molecular weight PEG, carbohydrates such as sorbitol or xylitol, most preferably the cryoprotectant is glycerol.

### DETAILED DESCRIPTION OF THE INVENTION

The term “apo” as used herein is taken to mean macromolecule and in particular any protein (or named protein) that is detached from a/its ligand(s) and/or prosthetic group(s).

The term “buffer” as used herein is taken to include any solution containing a weak acid and a conjugate base of this acid (or, less commonly, a weak base and its conjugate acid). Thus, a “buffer” as used herein resists change in its pH level when an acid or a base is added to it, because the acid neutralises an added base (or, less commonly, the base neutralises an added acid).

An “activity assay” as referred to herein with reference to PDE5 is taken to mean an in vitro assay of PDE inhibitory activities against cyclic guanosine 3′,5′-monophosphate (cGMP) and cyclic adenosine 3′,5′-monophosphate (cAMP) phosphodiesterases which can be determined by measurement of their IC₅₀ values (the concentration of compound required for 50% inhibition of enzyme activity). Preferably the required PDE enzymes can be isolated from a variety of sources, including human corpus cavernosum, human and rabbit platelets, human cardiac ventricle, human skeletal muscle and bovine retina, essentially by a modification of the method of Thompson W J and Appleman M M; Biochemistry 10(2),311-316, 1971, as described by Ballard S A et al.; J. Urology 159(6), 2164-2171, 1998. In particular, cGMP-specific PDE5 and cGMP-inhibited cAMP PDE3 can be obtained from human corpus cavernosum tissue, human platelets or rabbit platelets.

Assays can be performed either using a modification of the “batch” method of Thompson, W J et al.; Biochemistry 18(23), 5228-5237, 1979, essentially as described by Ballard S A et al.; J. Urology 159(6), 2164-2171, 1998, or using a scintillation proximity assay for the direct detection of [³H]-labelled AMP/GMP using a modification of the protocol described by Amersham plc under product code TRKQ7090/7100. In summary, for the scintillation proximity assay, the effect of PDE inhibitors are investigated by assaying a fixed amount of enzyme in the presence of varying inhibitor concentrations and low substrate (cGMP or cAMP in a 3:1 ratio unlabelled to [³H]-labeled at a concentration of ˜1/3 Kₘ or less), such that IC₅₀≅Kᵢ. The final assay volume is made up to 100 μl with assay buffer [20 mM Tris-HCl pH 7.4, 5 mM MgCl₂, 1 mg/ml bovine serum albumin]. Reactions are initiated with enzyme, incubated for 30-60 min at 30° C. to give <30% substrate turnover and terminated with 50 μl yttrium silicate SPA beads (containing 3 mM of the respective unlabelled cyclic nucleotide for PDE5). Plates are re-sealed and shaken for 20 min, after which the beads are allowed to settle for 30 min in the dark and then counted on a TopCount plate reader (Packard, Meriden, Conn.). Radioactivity units are converted to % activity of an uninhibited control (100%), plotted against inhibitor concentration, and inhibitor IC₅₀ values obtained using the ‘Fit Curve’ Microsoft Excel extension.

The term “precipitant” as used herein is taken to include any substance that, when added to solution comprising a biological molecule, causes the biological molecule to precipitate or crystallise from the solution.

The term “complex” as used herein is taken to mean a biological macromolecule, preferably a protein, with ligand(s) bound and may be formed before, during or after protein crystallisation.

The term “soaking” or “capable of being soaked” as used herein is taken to mean a process of placing a crystal in an aqueous solution containing a chemical compound, (usually) small molecule (e.g. inhibitor), or adding a chemical compound to an aqueous solution containing a crystal such that the compound may be able to enter the crystal lattice by diffusion and may interact (e.g. contact and bond with) with the molecules comprising the lattice preferably, where the molecules comprising the lattice are protein molecules, to form a protein-ligand complex, most preferably where the molecules comprising the lattice are protein molecules and the ligand or compound become bound to the active site of the protein molecule. The compound may be added to the aqueous solution in solid form, or it may be dissolved in a suitable solvent, preferably di-methyl-sulphoxide (DMSO).

The term “soakable crystal” as used herein is taken to mean a crystal into which a small molecule or ligand may be soaked without significant disruption of the lattice such that the ligand can enter and pass through the crystal lattice and have access to, and may associate with, the molecules comprising the lattice. Preferably a soakable crystal is a crystal which is ameanable to the soaking process in which a small molecule or ligand either in solution or in solid form is added to the solution containing the crystal. The small molecule may then enter the crystal lattice by diffusion and associate with the molecules comprising the crystal lattice. It is preferable that the soakable crystal binds the ligand without disruption of the lattice or cracking of the crystal and that the lattice symmetry and crystal parameters are not significantly altered by the soaking and ligand binding process. It is preferable that the soakable crystal diffracts X-rays to atomic resolution, preferably to beyond 3.5 Å, more preferably beyond 2.5 Å, most preferably 1.5 Å, after undergoing the soaking process.

The term “stabilising solution” as used herein is taken to mean a solution into which a crystal may be transferred for the purposes of further manipulation, for example for soaking, and in which the crystal retains its structural integrity and does not crack or dissolve or alter significantly in its crystal parameters, symmetry or cell dimensions. Preferably the stabilising solution comprises some or all of the chemical constituents from which the crystal was grown and is of the approximately the same, and more preferably identical, pH.

The term “cryoprotectant” as used herein is taken to mean a chemical compound which, when added to a solution, allows the solution to be rapidly frozen without the formation of ice crystals. Such a cryoprotectant is preferably an alcohol such as ethanol or a carbohydrate such as xylitol or sorbitol, but may also be 2-methyl-2,4-pentanediol (MPD) or other organic polymers e.g. lower molecular weight PEG. Most preferably the cryoprotectant is glycerol.

The term “co-crystallisation” as used herein is taken to mean crystallisation of a pre-formed comple of a macromolecule with its ligand i.e. a protein/small molecule complex.

The terms “mutant”, “variant”, “homologue”, “analogue”, “derivative” or “fragment”, are in relation to the amino acid sequence of the PDE5 protein or polypeptide sequence which is used to produce the crystal of the present invention. The terms include any substitution of, variation of, modification of, replacement of, deletion of, or addition of one (or more) amino acids from (or to) the sequence providing the resultant PDE5 is capable of being crystallised.

Typically, for the “mutant”, “variant”, “homologue”, “analogue”, “derivative” or “fragment” in relation to the amino acid sequence of the protein or polypeptide of the PDE5 of the crystal of the present invention, the types of amino acid substitutions that could be made should maintain the hydrophobicity/hydrophilicity of the amino acid sequence. Amino acid substitutions may be made, for example from 1, 2 or 3 to 10, 20 or 30 substitutions, provided that the modified PDE5 retains the ability to be crystallised in accordance with present invention. Amino acid substitutions may include the use of non-naturally occurring analogues.

In relation to amino acid sequences, the term “variant” as used herein refers to additions, deletions or substitutions of amino acid residues comprised within the wild-type amino acid sequence or fragment thereof. Preferably, a variant in relation to the amino acid sequence of the crystal of the PDE5 of the present invention could include the deletion or substitution of the histidine (His/H) residue as shown emboldened and underlined in SEQ ID NO: 1 (HRGVNNSYIQRSEHPLAQLYCHSIME), which sequence is comprised in the protein or polypeptide of PDE5 of the crystal of the present invention. Replacement of said histidine (H) residue is preferably by way of incorporating one or more amino acid residues (other than histidine), preferably wherein said amino acid residues are neutral or non-polar.

The terms “mutant”, “variant”, “homologue”, “analogue”, “derivative” or “fragment” in relation to the nucleotide sequence coding for the PDE5 of the crystal of the present invention include any substitution of, variation of, modification of, replacement of, deletion of, or addition of, one (or more) nucleotide from (or to) the sequence providing the resultant nucleotide sequence codes for, or is capable of coding for, a PDE5 which is capable of being crystallised.

In relation to nucleotide sequences, the term “variant” as used herein refers to additions, deletions or substitutions of nucleotides of the wild-type nucleotide sequence or fragment thereof.

The term “fragment” as used herein refers to any portion of the PDE5 amino acid sequence as defined in the present invention provided the resultant PDE5 comprising said PDE5 portion is capable of being crystallised. Thus, the term “fragment” also includes PDE5, which comprises any portion of SEQ ID NOS: 1, 2, 3, 4, 5, or 6.

An example of a specific fragment of SEQ ID NO: 6 (full-length “loop-swapped” PDE5 sequence) according to the present invention could be SEQ ID NO: 5 (“loop-swapped” PDE5 catalytic domain). Moreover, an example of a specific fragment of SEQ ID NO: 5 (“loop-swapped” PDE5 catalytic domain) according to the present invention could be SEQ ID NO: 4 (PDE4 “loop region”; HPGVSNQFLINTNSELALMYNDESVLE).

The term “analogue” as used herein means a sequence similar to the amino acid sequence of the PDE5 polypeptide of the crystal of the present invention or of any one of SEQ ID NOS: 1, 2, 3, 4, 5 or 6, but wherein non-detrimental (i.e. not detrimental to the PDE5's capability of being crystallised) amino acid substitutions or deletions have been made.

The term “derivative” as used herein in relation to the amino acid sequence of the PDE5 of the crystal of the present invention, or of any one of SEQ ID NOS: 1, 2, 3, 4, 5 or 6, includes chemical modification of PDE5. Illustrative of such modifications would be replacement of hydrogen by an alkyl, acyl, or amino group.

The term “heavy atom derivative” as used herein in relation to crystals refers to a crystal comprising macromolecules in the crystal lattice which are modified by the inclusion of a heavy atoms (i.e. of significantly greater atomic mass than the atoms common to organic macromolecules, e.g. carbon, nitrogen, oxygen, phosphorous, etc.) into their structure. This is commonly achieved using ionic compounds such as salts of mercury, platinum, gold or silver which may covalently bond to certain groups on the macromolecule either during co-crystalisation with the ionic compound or by soaking the compound into the crystal. Other processes may also be used such as derivitisation with seleno-methionine or with noble gases such as Xenon. The purpose of such heavy atom derivatives of crystals is to provide phasing information for structure solution.

As used herein a “deletion” is defined as a change in either nucleotide or amino acid sequence in which one or more nucleotides or amino acid residues, respectively, are absent.

As used herein an “insertion” or “addition” is a change in a nucleotide or amino acid sequence, which has resulted in the addition of one or more nucleotides or amino acid residues, respectively, as compared to the sequences of the naturally occurring PDE5.

As used herein “substitution” results from the replacement of one or more nucleotides or amino acids by different nucleotides or amino acids, respectively.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The term “homologue” covers homology specifically with respect to protein structure, primary secondary, tertiary and quaternary, and covers any structural PDE5 homologue that is capable of being crystallised.

With respect to homology of the amino acid sequences detailed herein, preferably there is at least 70%, more preferably at least 75%, more preferably at least 80%, yet more preferably at least 85%, even more preferably at least 90% homology to SEQ ID NOS: 1, 2, 3, 4, 5 or 6. More preferably there is at least 95%, and most preferably at least 98%, homology to SEQ ID NOS: 1, 2, 3, 4, 5 or 6.

With respect to homology of the nucleotide sequences coding for the amino acid sequences detailed herein, preferably there is at least 70%, more preferably at least 75%, more preferably at least 80%, yet more preferably at least 85%, even more preferably at least 90% homology to the nucleotide sequences which code for SEQ ID NOS: 1, 2, 3, 4, 5 or 6. More preferably there is at least 95%, and most preferably at least 98%, homology to the nucleotide sequences which code for SEQ ID NOS: 1, 2, 3, 4, 5 or 6.

The term “homologue” with respect to the nucleotide sequence of the PDE5 as defined in the present invention and the amino acid sequence of the PDE5 as defined in the present invention may be synonymous with allelic variations of the sequences.

In particular, the term “homology” as used herein may be equated with the term “identity”. Here, sequence homology with respect to, for example, the amino acid sequence of PDE5 of the crystal of the present invention can be determined by a strict comparison of any one or more of the sequences with another sequence to see if that other sequence has at least 70% identity to the sequence(s). Relative sequence homology (i.e. sequence identity) can also be determined by commercially available computer programs that can calculate percentage (%) homology between two or more sequences. A typical example of such a computer program is CLUSTAL.

Percentage homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for off-line and on-line searching (see Ausubel et al., 1999 ibid, pages 7-58 to 7-60). However, for some applications it is preferred to use the GCG Bestfit program.

Although the final percentage homology can be measured in terms of identity, in some cases, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate percentage homology, preferably percentage sequence identity. The software typically does this as part of the sequence comparison and generates a numerical result.

As indicated, for some applications, sequence homology (or identity) may be determined using any suitable homology algorithm, using for example default parameters. For a discussion of basic issues in similarity searching of sequence databases, see Altschul et al (1994) Nature Genetics 6:119-129. For some applications, the BLAST algorithm is employed, with parameters set to default values. The BLAST algorithm is described in detail at http://www.ncbi.nih.gov/BLAST/blast_help.html. Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

Other computer program methods to determine identify and similarity between the two sequences include but are not limited to the GCG program package (Devereux et al 1984 Nucleic Acids Research 12: 387) and FASTA (Atschul et al 1990 J Molec Biol 403-410).

The amino acid sequence of the PDE5 of the present invention present invention may be produced by expression of a nucleotide sequence coding for the same in a suitable expression system.

In addition, or in the alternative, the protein itself could be produced using chemical methods to synthesize a PDE5 amino acid sequence, in whole or in part. For example, peptides can be synthesized by solid phase techniques, cleaved from the resin, and purified by preparative high performance liquid chromatography (e.g. Creighton (1983) Proteins Structures and Molecular Principles, W H Freeman and Co., New York, N.Y., USA). The composition of the synthetic peptides may be confirmed by amino acid analysis or sequencing (e.g. the Edman degradation procedure).

Direct peptide synthesis can be performed using various solid-phase techniques (Roberge J Y et al, Science, Vol 269, 1995, pp. 202-204) and automated synthesis may be achieved, for example, using the ABI 431 A Peptide Synthesizer (Perkin Elmer, Boston, Mass., USA) in accordance with the instructions provided by the manufacturer. Additionally, the amino acid sequence of PDE5, or any part thereof, may be altered during direct synthesis and/or combined using chemical methods with a sequence from other subunits, or any part thereof, to produce a variant polypeptide.

Protein Engineering of PDE5*

Analysis of the catalytic domain of wild-type PDE5 protein by mass spectrometry and SDS-PAGE (data not shown) shows that the protein is cleaved within the region of residues 664-682. High concentrations of protease inhibitors provide some stabilisation of the protein, but cleavage still prevents reproducible crystallisation.

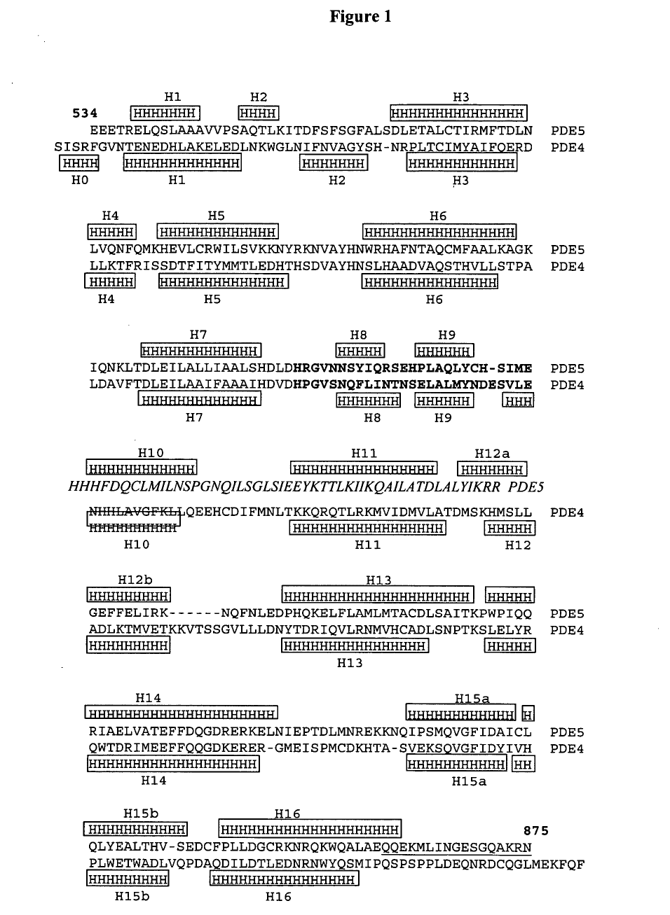

An engineered form of the catalytic domain of PDE5, called ‘PDE5*’, has been produced where the 657-682 region of PDE5 has been replaced by the same region in PDE4 producing a chimeric construct, (see FIG. 1 for sequence alignment of this region). The C-terminus of this construct is also truncated (C-term 858) compared to the wild-type structure (C-term 875). Hereafter this engineered construct will be referred to as ‘PDE5*’.

This engineered protein has been shown to be stable to degradation by mass spectrometry and SDS-PAGE (data not shown). The protein shows improved biophysical properties allowing an alternative purification protocol to be developed.

The new protocol utilises binding to a Blue sepharose column and specific elution with cGMP. The wild-type protein had been shown not to bind to this column probably due to the disorder of the structure around the protease cleavage site. This PDE5* protein was used to produce crystals with inhibitors which diffract to higher resolution and have no disordered regions. The protein has also been used reproducibly to produce crystals with further inhibitors which routinely diffract to 1.8 Å resolution or higher, making it an improved protein for use in structure based drug design.

The Structure of PDE4 and Wild Type PDE5 Catalytic Domains

(This is also detailed in Application Number PCT/IB02/04426, page 28, line 19 to page 29, line 17 and is incorporated herein by reference.)

The structure of the catalytic domain of PDE4b was published (Xu et al. 2000). A topological comparison of the PDE5 catalytic domain with the structures in the Protein Data Bank (PDB) does not reveal significant additional homology with other known protein structures except for the PDE4 structure. Comparisons between the two structures have been made (FIG. 1 of the present application shows a sequence and secondary structural alignment of the two proteins). The structure and domain arrangement is virtually identical to that of PDE4, save that the second sub-domain highlighted in PDE4 is only partially present in the PDE5 structure, as detailed below.

The structure is composed of a single domain of 15 α helices arranged in a compact fold (FIG. 2 of the present application). Within the overall domain, three sub-domains can also be defined. Helices 1 (H1 539-545) and 2 (H2 551-554) lie on the exterior of the protein and comprise the N-terminal region of the construct. These two helices do not overlay with the equivalent ones (H0, H1 and H2) in the PDE4 structure. This region is not well conserved across the PDE protein family. Helices 3 (H3 568-582), 4 (H4 584-588), 5 (H5 592-604), 6 (H6 615-631) and 7 (H7 640-652) form the first sub-domain of the protein and are contained within the core of the protein. There is no observable electron density for helices 8 and 9 based on the PDE4 nomenclature. Helix 10 (H10 684-694) is again on the exterior and forms the dimer interface within the structure. Helices 10 and 11 (H11 706-721) are the visible portion of the second sub-domain. Helices 12 (H12a 725-731, H12b 733-741), 13 (H13 749-765), 14 (H14 772-797), 15 (H15 813-824), 16 (H16 826-836) and 17 (H17 841-861) form the third sub-domain of the protein. It should be noted that in PDE5 helix H12 is not a contiguous helix as in PDE4 but is composed of two short helices with a kink in the middle and helix H15 is a contiguous helix in PDE5 but not in PDE4.

Structure of PDE5* Catalytic Domain Complexed with UK-088,800

The structure of the catalytic domain of PDE5* protein complexed with UK-088,800 was determined by molecular replacement using one molecule of the protein structure from the PDE5* complex with Sildenafil (as described in Application Number PCT/IB02/04426: Example 11, “crystallisation of PDE5* with Sildenafil”, page 46, lines 8 to 25; Example 15, “data collection, structure determination and refinement PDE5* with Sildenafil”, page 50, line 9 to page 51, line 6 and Table 6, “atomic co-ordinates for baculovirus-expressed PDE5* complex with Sildenafil”, pages 328 to 408; incorporated herein by reference) as a basis for the search model, the co-ordinates for which are included in Table 3 of the present application. The PDE5* catalytic domain crystallises in the space group C2 as a monomer with one molecule present in the asymmetric unit. The C2 form of the PDE5* catalytic domain crystallises in the space group C2 with approximate cell dimensions a=55.97 Å, b=76.55 Å, c=80.50 Å and β=103.09. They contain one molecule per asymmetric unit and have a solvent content of 44% (Matthews, 1969). The structure of the PDE5* comprises 17 α helices and the overall fold is very similar to the wild-type structure with a number of important differences. The major difference in the structure is the presence of helices H8 and H9 composed of the swapped portion from PDE4, residues 657-682. These fold in an identical way to that observed in the PDE4 structure and complete the second sub-domain of the protein. The entire C-terminal region of this construct can also be built into the electron density leaving just three disordered residues at the N-terminus of this structure. This is likely to contribute to its enhanced properties for crystallisation.

PDE5*: Active Site and Protein-inhibitor Interactions

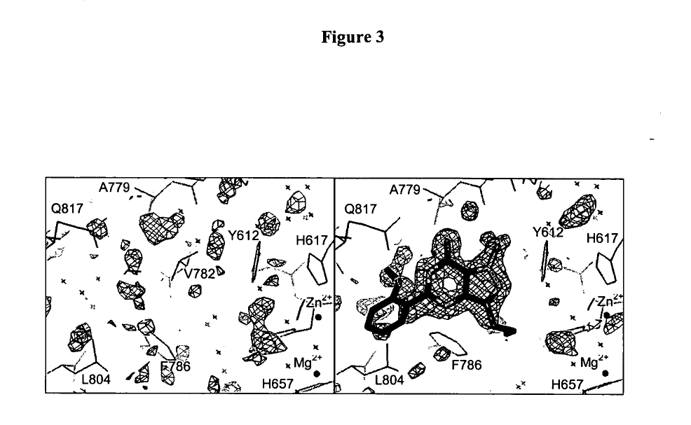

UK-088,800 occupies the same region of the active site as observed for the previously described UK-092,480, Sildenafil, as detailed below (Application Number PCT/IB02/04426: FIG. 4, “A view of compound Sildenafil bound to loop swapped PDE5 (PDE5*), page 425; incorporated herein by reference) and demonstrates the same mainly hydrophobic interactions with the protein (FIG. 3 “Comparison of the fo-fc electron density map contoured at 2.5σ of apo PDE5* (left) and PDE5* soaked with UK-088,800 (right), at the active site region” of the present application). The same two direct hydrogen bonds observed in the complex of PDE5* with Sildenafil are also observed between Gln 817 of the protein and inhibitor (Oε1-N4 2.9 Å and Nε2-O1 3.1 Å).

Carbon atom C7 of the inhibitor points into a small hydrophobic pocket formed by Leu 765, Ala 767 and Ile 768. These residues, together with Phe 820, form a planar face to the binding site against which the purine ring of the inhibitor stacks. The opposite side of the purine packs against Val 782. The C10 propyl substituent form good van der Waals contacts with Phe 786. Phe 786 packs against Leu 804 which in turn forms additional hydrophobic interactions with the phenyl moiety of the inhibitor. The O-alkyl moiety occupies a small pocket bounded by Ala 779, Phe 786, Ala 783, Val 782, Leu 804, Ile 813, Met 816 and Gln 817. There is no direct interaction between the inhibitor and the zinc ion found in the active site. The structure confirms the competitive nature of the mode of inhibition of UK-088,800 by binding in the active site, therefore blocking access for the cGMP substrate (which has also been modelled—data not included).

The main difference comparing Sildenafil and UK-088,800 is that the latter inhibitor lacks the sulphonylpiperazine present in Sildenafil which points out of the active site.

Wild-type PDE5-Sildenafil Complex: Active Site and Protein-inhibitor Interactions

(This is detailed in Application Number PCT/IB02/04426, page 30, line 8 to page 31, line 1, and is incorporated herein by reference.)

The active site lies mainly within the third sub-domain of the protein and is bounded by helices H15, H14, the C-terminus of H13, and the C-terminus of H11, along with the loop region between H11 and H12a. The majority of the interactions between the inhibitor and the protein are hydrophobic in nature, with only two direct hydrogen bonds observed (Application Number PCT/IB02/04426: FIG. 3, page 424, incorporated herein by reference). The first is between N17 of the purine ring of the inhibitor and Oε1 of Gln 817 (2.8 Å) and the second is from the adjacent oxygen atom O16 of the inhibitor to Nε2 of the same residue Gln 817 (3.1 Å).

Carbon atom C12 of the inhibitor points into a small hydrophobic pocket formed by Leu 765, Ala 767 and Ile 768. These residues, together with Phe 820, form a planar face to the binding site against which the purine ring of the inhibitor stacks. The opposite side of the purine packs against Val 782. The C5 propyl substituent forms good van der Waals contacts with Val 782 and Phe 786 and Tyr 612. Phe 786 and Leu 804 form additional hydrophobic interactions with the phenyl moiety of the inhibitor. The O-alkyl moiety occupies a small pocket bounded by Ala 779, Phe 786, Ala 783, Val 782, Leu 804, Ile 813, Met 816 and Gln 817. The sulphonamide group points out towards the solvent whilst the piperazine ring is bounded by the extended residues 662-665, although whether the conformation of this part of the structure is unaffected by the chain break is questionable. There is no direct interaction between the inhibitor and the zinc ion found in the active site. The structure confirms the competitive nature of the mode of inhibition of Sildenafil by binding in the active site therefore blocking access for the cGMP substrate (which has also been modelled—data not included).

Wild Type PDE5-Sildenafil Complex: Metal Ions in the Active Site

Only one zinc atom is present in the active site of this structure. The co-ordination of the ion within the active site is also consistent with that expected for zinc. The metal is co-ordinated by His 653 (Nε2-Zn 2.0 Å), His 617 (Nε2-Zn 2.1 Å), Asp 764 (OD2-Zn 2.2 Å) and also Asp 654 (OD2-Zn 2.2 Å). These residues are completely conserved across the PDE gene family. There is no evidence of a second metal ion in the active site.

The present invention will now be described, by way of example only, with reference to the accompanying Sequence Listing and Figures, in which:
SEQ ID NO: 1 shows the amino acid sequence of the loop region from PDE5.
SEQ ID NO: 2 shows the amino acid sequence of the wild-type PDE5 catalytic domain.
SEQ ID NO: 3 shows the amino acid sequence of the full-length wild-type PDE5 sequence.
SEQ ID NO: 4 shows the amino acid sequence of the loop region of PDE4.
SEQ ID NO: 5 shows the amino acid sequence of the loop-swapped PDE5 catalytic domain=PDE5*.
SEQ ID NO: 6 shows the amino acid sequence of full-length PDE5 sequence comprising PDE5*.
SEQ ID NOS: 7-14 are oligonucleotide primers.

FIG. 1 shows an alignment of PDE5 (upper sequence) and PDE4b (lower sequence) catalytic domains. Positions and numbering of helices from the structures are marked for each. Residues in bold show a sequence alignment for the engineered region. The sequence from PDE4 has been used to replace the corresponding region in PDE5. This results in a residue insertion in this region. Underlining highlight C-terminal region absent in PDE5*.

FIG. 2 shows a ribbon representation of the overall fold of proteins showing secondary structure elements. The inhibitor is shown in an all atom stick representation and the metal ions as spheres. (A)=PDE4b, (B)=wild-type PDE5+Sildenafil, (C)=“loop-swapped” PDE5 (PDE5*)+Sildenafil. Helices are numbered using PDE4 structure as reference. Helices H0-H7 form sub-domain 1, helices H8-H11 form sub-domain 2, and helices H12-H16 form sub-domain 3.

FIG. 3: Comparison of the fo-fc electron density map contoured at 2.5σ of apo PDE5* (“loop-swapped” PDE5) (left) and PDE5* soaked with UK-088,800 (right), at the active site region.

FIG. 4: A two dimensional representation of the structure of UK-088,800, 5-(2-ethoxyphenyl)-1-methyl-3-propyl-1,6-dihydro-7H-pyrazolo[4,3-d]pyrimidin-7-one (only the Hydrogen on N4 is illustrated).

Experimental Section

### EXAMPLE 1

### Construction and Expression of PDE5 Wild-type Catalytic Domain (E534-N875)

Oligonucleotide primers were designed from the sequence of human PDE5 (Accession number=AB001635). DNA fragments were generated by PCR amplification from a full-length PDE5 clone. The following oligonucleotides were used:

| | |
|---|---|
| PDE5 5′ Untagged Oligo: | |
| CGTGAATTCATGGAGGAAGAAACAAGAGAGCTAC | (SEQ ID NO: 7) |
| PDE5 3′ Long Oligo: | |
| CGTTCTAGACTATCAGTTCCGCTTGGCCTGGCCGCT | (SEQ ID NO: 8) |
| TTCCCC | |

The PCR reaction was carried out for 30 cycles in a total volume of 50 μl in a solution containing 1.5 mM MgCl₂, 200 μM dNTPs, 50 pmol of each primer and 2.5 units of Expand DNA polymerase (Roche, East Sussex, UK). Each cycle was 94° C., 1 min, 50° C., 1 min and 72° C., 2 mins.

The final amplified DNA fragments for both constructs were separated on a 1% agarose gel and purified using a QIAquick gel extraction kit (Qiagen, West Sussex, UK). The fragment was then digested using EcoRI and XbaI, and ligated into pFastbacl EcoRI/XbaI-digested vector (Life Technologies, Paisley, UK). The ligation was carried out at 12° C. for 16 hours. The ligation mix was then electroporated into E. coli (TOP 10) (Invitrogen, Gronigen, The Netherlands).

Clones containing the desired insert were selected by using 2YT plates containing 100 μg/ml ampicillin and checked using endonuclease digestion for presence of correct size insert. DNA sequence analysis was carried out by Lark (Saffron Waldon, UK).

Recombinant bacmid DNA was produced by transforming E.coli DH10BAC™ with pFastbacl::PDE5 catalytic domain (534-875) plasmid DNA. This was carried out according to the method shown in the Bac to Bac™ baculovirus expression manual (Life Technologies, Paisley, UK). PCR analysis was used to verify successful transposition to the bacmid using pUC/M13 amplification primers (Invitrogen, Gronigen, The Netherlands).

Generation of primary baculovirus stocks was carried out by transfection using Sf-9 insect cells. Bacmid DNA containing the correct insert was mixed with CELLFECTIN™ transfection reagent (Life Technologies, Paisley, UK) and added to a monolayer of Sf-9 insect cells using SF-900II serum free medium (Invitrogen, Gronigen, The Netherlands). Following 72 hours incubation at 27° C. the supernatant was harvested as the initial baculovirus stock. This stock was amplified by adding the initial virus stock into a suspension of Sf-9 insect cells at 1×10⁶ cells/ml in 1 litre Erlenmeyer flasks (Corning Life Sciences, New York, USA), at an agitation of 125 rpm at 27° C. After 6 days post infection the supernatant was harvested by centrifugation and stored at 4° C. as the working virus stock. The titre of this working stock was determined by conventional plaque assay analysis as in the Bac to Bac baculovirus expression manual (Invitrogen, Gronigen, The Netherlands).

Protein expression was optimised in Erlenmeyer flask cultures using Sf-9 and T.ni High5 insect cell cultures looking at different multiplicity's of infection (MOI) and harvest times, the optimal conditions found were then scaled up into fermenters.

The fermenters used were autoclavable Applikon 3 litre stirred vessels controlled using Applikon 1030 biocontrollers. Inoculum of T.ni High5 cells was initially prepared from shake flask cultures. The fermenter was inoculated with 5×10⁵ cells/ml, with an initial working volume of 1.8 litres made up in Excel 405 serum free medium (JRH Biosciences, Kansas, USA). Temperature was controlled at 27° C., dissolved oxygen concentration controlled at 60% and pH was measured but not controlled. Oxygen concentration was controlled throughout. Agitation was set at 150 rpm with a double impeller system of marine impeller within the culture and Rushton impeller at the liquid/headspace interface. Aeration was continuous to the headspace at 0.5 l/min.

When the viable cell density reached 2×10⁶ cells/ml the culture was infected using an MOI of 1 from the titred baculovirus working stock. Harvest time for the culture was 48 hours post infection. This was achieved by centrifugation at 2000 g for 15 mins; the insect cell pellet was then stored at −80° C. prior to purification.

### EXAMPLE 2

### Construction and Expression of His6-tagged PDE5 Wild-type Catalytic Domain (E534-N875)

Oligonucleotide primers were designed from the sequence of human PDE5 (=PDE5A1 isoform; Accession number=AB001635). DNA fragments were generated by PCR amplification from a full-length PDE5 clone. The following oligonucleotides were used:

| | |
|---|---|
| PDE5 5′ His6 Oligo | |
| CGTGAATTCATGCATCATCATCATCATCATCTTCTG | (SEQ ID NO: 9) |
| GTTCCGCGTGGATCTGCGCCCGAGGAAGAAACAAGA | |
| GAGCTAC | |
| PDE5 3′ Long Oligo | |
| CGTTCTAGACTATCAGTTCCGCTTGGCCTGGCCGCT | (SEQ ID NO: 8) |
| TTCCCC | |

The PCR reaction was carried out for 30 cycles in a total volume of 50 μl in a solution containing 1.5 mM MgCl₂, 200 μM dNTPs, 50 pmol of each primer and 2.5 units of Expand DNA polymerase (Roche, East Sussex, UK). Each cycle was 94° C., 1 min, 50° C., 1 min and 72° C., 2 mins.

The final amplified DNA fragments for both constructs were separated on a 1% agarose gel and purified using a QIAquick gel extraction kit (Qiagen, West Sussex, UK). The fragment was then digested using EcoRI and XbaI, and ligated into pFastbacl EcoRI/XbaI-digested vector (Life Technologies, Paisley, UK). The ligation was carried out at 12° C. for 16 hours. The ligation mix was then electroporated into E. coli (TOP 10) (Invitrogen, Gronigen, The Netherlands).

Clones containing the desired insert were selected by using 2YT plates containing 100 μg/ml ampicillin and checked using endonuclease digestion for presence of correct size insert. DNA sequence analysis was carried out by Lark (Saffron Waldon, UK).

Methods to generate the recombinant baculovirus were as those for wild-type PDE5 catalytic domain (see EXAMPLE 1).

Expression optimisation again showed T.ni High5 insect cells to give the best expression. Therefore baculovirus expression in fermenters was carried out using the same procedures as for the previous construct.

### EXAMPLE 3

### Construction and Expression of PDE5* (E534-E858) in Baculovirus

The PDE5* construct was produced by using overlap extension PCR where the following oligonucleotides were used:

| | |
|---|---|
| A: | |
| CGTGAATTCATGGAGGAAGAAACAAGAGAGCTAC | (SEQ ID NO: 7) |
| B: | |
| CAAAGAAAGTTCTGAATTTGTGTTGATGAGAAAC | (SEQ ID NO: 10) |
| TGATTGGAGACTCCAGGATGATCCAAATCGTGGC | |
| TTAG | |
| C: | |
| ATCAACACAAATTCAGAACTTGCTTTGATGTATA | (SEQ ID NO: 11) |
| ATGATGAATCTGTGTTGGAACACCATCATTTTGA | |
| CCAG | |
| D: | |
| CGTTCTAGACTATCATTCTGCAAGGGCCTGCCAT | (SEQ ID NO: 12) |
| TTCTG | |

Initial DNA fragments were generated using oligonucleotides A+B and C+D with the same template DNA as for the wild-type PDE5 catalytic domain construct. The PCR reaction was carried out for 30 cycles in a total volume of 50 μl in a solution containing 1.5 mM MgCl₂, 200 μM dNTPs, 50 pmol of each primer and 2 units of Expand DNA polymerase (Roche, East Sussex, UK). Each cycle was 94° C., 1 min, 50° C., 2 min, and 72° C., 3 min. In the second round of PCR, DNA products from PCR A+B and C+D were used as template DNA with the oligonucleotides A+D used to amplify the full-length construct. The PCR conditions were the same as the initial PCR reaction. This generates a construct with the PDE4 swapped region and a C-terminal truncation (C-term 858) as compared to PDE5 catalytic domain (C-term 875).

Methods to generate the recombinant baculovirus were as those for wild-type PDE5 catalytic domain (see EXAMPLE 1).

Expression optimisation again showed T.ni High5 insect cells to give the best expression. Therefore baculovirus expression in fermenters was carried out using the same procedures as for the previous construct.

### EXAMPLE 4

### Construction and Expression of PDE5* (E534-E858) in E. coli

The PDE5* construct in E. coli was produced by using PCR where the following oligonucleotides were used and the template DNA being pFastbacl::PDE5* plasmid DNA (sequence verified), produced in EXAMPLE 3.

| | |
|---|---|
| E: | |
| CGTCATATGGAGGAAGAAACAAGAGAGCTAC | (SEQ ID NO: 13) |
| F: | |
| CGTCTCGAGCTATCATTCTGCAAGGGCCTGCCAT | (SEQ ID NO: 14) |
| TTCTG | |

The PCR reaction was carried out for 30 cycles in a total volume of 50 μl in a solution containing 1.5 mM MgCl₂, 200 μM dNTPs, 50 pmol of each primer and 2.5 units of Expand DNA polymerase (Roche, East Sussex, UK). Each cycle was 94° C., 1 min, 50° C., 1 min and 72° C., 2 mins.

The final amplified DNA fragment was separated on a 1% agarose gel and purified using a QIAquick gel extraction kit (Qiagen, West Sussex, UK). The fragments were then digested using Ndel and Xhol, and ligated into pET21C (Novagen, Nottingham, UK) Ndel/Xhol-digested vector. The ligation was carried out at 12° C. for 16 hours. The ligation mix was then electroporated into E. coli (TOP 10) (Invitrogen, Gronigen, The Netherlands).

Clones containing the desired insert were selected by using 2YT plates containing 100 μg/ml ampicillin. Plasmid DNA was also checked using endonuclease digestion for presence of correct size insert. DNA sequence analysis was carried out by Lark (Saffron Waldon, UK).

The correctly sequenced plasmid DNA was then electroporated into E. coli BL21 (DE3) (Novagen, Nottingham, UK) for expression. Expression was carried out in 7 litre Applikon fermenters using 5 litre 2YT broth containing 100 μg/ml carbenicillin as the medium. Agitation was set at 1000 rpm using a double rushton impeller assembly and aeration to the sparger at 2 litres/min. The fermenter was inoculated with an overnight shake flask culture grown at 37° C. and 200 rpm, the inoculation density was 1% vol/vol. The fermentation was pH controlled at 7.2 using 20% vol/vol NH₄OH solution and temperature initially set to 37° C. When the OD_{600 nm} reached 1.5 the temperature set-point was reduced to 25° C. and then the culture was induced with EPTG at a final concentration of 1 mM. The fermentation was then harvested 4 hours post-induction by batch centrifugation (8,000 rpm for 10 minutes). The final pellet was then frozen (−80° C.) to await subsequent purification.

### EXAMPLE 5

### Purification of PDE5* Catalytic Domain

Pellet from the E. coli fermentation was resuspended into 10 mls lysis buffer per gram wet cell weight and mechanically broken using a continuous cell disrupter (Constant Systems, Warwickshire, UK) at a pressure of 20 kpsi. The lysis buffer consisted of 50 mM Tris HCl (pH 7.5), 100 mM NaCl, 1 mM DTT containing EDTA-free protease inhibitor cocktail tablets (Roche, East Sussex, UK) and 10 μM E-64. The lysate was chilled and centrifuged at 14000 g for 45 min to remove cell debris. All purifications were subsequently carried out using an Äkta Explorer purification system (Amersham Pharmacia, Buckinghamshire, UK). The supernatant was applied to a 50 ml Q-sepharose fast-flow column (Amersham Pharmacia, Buckinghamshire, UK) at 5 ml/min with the flow-through collected. The flow-through sample was then applied at 50 ml/min to a 2 litre G-25 superfine desalting column (Amersham Pharmacia, Buckinghamshire, UK) pre-equilibrated in Blue sepharose buffer A (50 mM Bis-Tris (pH 6.4), 50 mM NaCl, 2 mM EDTA, 2 mM EGTA and 1 mM DTT). The protein fraction was eluted in Blue sepharose buffer A.

The next column step was carried out in series, loading the sample initially onto a 20 ml SP-sepharose high performance column (Amersham Pharmacia, Buckinghamshire, UK) then flow-through from this directly onto a 10 ml Blue sepharose fast-flow column (Amersham Pharmacia, Buckinghamshire, UK) at a flow-rate of 2 ml/min. Once loading was complete, the SP-sepharose column was taken out of line and the Blue sepharose column washed with 5 column volumes of Blue sepharose buffer A. The column was washed with Blue sepharose buffer A containing 1 M NaCl until the absorbance 280 nm reached baseline and then washed with 5 column volumes of Blue sepharose buffer A. PDE5* protein was step-eluted using Blue sepharose buffer containing 20 mM cGMP (Na-salt) (Sigma, Dorset, UK). Fractions were assayed on Tris-glycine SDS gels (Invitrogen, Gronigen, The Netherlands) and pooled accordingly. These fractions were concentrated to 2.5 mg/ml using centrifugal concentrators (Vivascience, Gloucestershire, UK) and loaded at 1.5 ml/min onto a Superdex-200 prep grade 26/60 column pre-equilibrated with 50 mM Bis-Tris (pH 6.8), 500 mM NaCl, 1 mM DTT and 2 μM E-64. The eluted fractions were analysed on Tris-glycine SDS PAGE gels.

### EXAMPLE 6

### Crystallisation of PDE5*

The PDE5* fractions from the final gel filtration column were pooled (total volume of 25 mls) and the protein concentration was assayed (0.2 mg/ml). The protein solution was supplemented with 10 μM E-64 and 1 mg/ml leupeptin (Sigma, Dorset, UK). The solution was concentrated to 10 mg/ml using a Centriprep 10 kDa Molecular weight cut-off centrifugal concentrator (Amicon Bioseparations, Maine, USA) at 3,000 rpm, 4° C. Prior to crystallisation, the protein solution was centrifuged for 5 min at 14,000 rpm in an Eppendorf centrifuge.

Hanging drop vapour diffusion crystallisation trials were set up at 4° C. Drops comprised of 2 μl reservoir buffer mixed with 2 μl protein solution were suspended on cover slips over 500 μl reservoir solutions containing 0.1M sodium HEPES pH 7.4, 20% Polyethylene glycol 4000 and 10% iso-Propanol. Plate-shaped crystals, up to 700 μm in largest dimension, grew after 1-3 days. Crystals were transferred to a 20 μl drop of 0.1M sodium HEPES pH 7.4 and 20% Polyethylene glycol 4000 in square microbridges. Crystals were then transferred to a solution of 0.1M sodium HEPES pH 7.4, 20% Polyethylene glycol 4000 with 15% glycerol and then frozen during X-ray data collection.

The crystals belong to space group C2 with cell dimensions a=55.97 Å, b=76.55 Å, c=80.50 Å and β=103.09. They contain one molecule per asymmetric unit and have a solvent content of 44% (Matthews, 1969).

### EXAMPLE 7

### Soaking Inhibitor into PDE5*

Plate-shaped crystals of apo PDE5* obtained according to methods detailed in EXAMPLE 6 were transferred to a 20 μl drop of 0.1M sodium HEPES and 20% Polyethylene glycol 4000 in square microbridges. To this, 1.0 μl of a 20 mg/ml concentration of inhibitor dissolved in DMSO was added. Crystals were then transferred to a solution of 0.1M sodium HEPES pH 7.4, 20% Polyethylene glycol 4000 with 15% glycerol and then frozen during X-ray data collection.

### EXAMPLE 8

### Data Collection, Structure Determination and Refinement of the Soakable C2 Crystal Form of PDE5*

The structure of the E. coli-engineered PDE5* was solved by molecular replacement (MR) using the coordinates of one subunit of PDE5* in the P2₁ crystal form (the crystals belong to the monoclinic space group P2₁, with unit cell dimensions a=54.93 Å, b=77.77 Å, c=82.05 Å, α=γ=90° 62 =100.955°) the coordinate set is detailed in Table 3. Molecular replacement was performed using AMORE (CCP4, J.Navaza 1994). The resulting map was of good quality and the structure was refitted using QUANTA® (Accelrys products).

A representative X-ray diffraction data set was collected with a Rigaku Saturn92 CCD detector on an in-house FR-D rotating anode (Rigaku), with Osmic mirrors (MSC). Data were processed using the CrystalClear/D*Trek processing package (Rigaku-MSC). Data collection statistics are summarised in Table 1 (Apo).

The crystals belong to space group C2 with cell dimensions a=55.97 Å, b=76.55 Å, c=80.50 Å and β=103.09. They contain one molecule per asymmetric unit and have a solvent content of 44% (Matthews, 1968).

Refinement was carried out in the resolution range 30-1.6 Å using CNX (Brünger et al., 1998) with the “mlf” maximum likelihood target function. Partial structure factors from a flat bulk-solvent model and anisotropic B-factor correction were supplied throughout the refinement. The R-factor for the refined model is 0.231 (free R-factor, 5% of data, 0.267) for all data in the resolution range 30-1.6 Å. The refinement statistics are summarised in Table 2 (Apo).

The co-ordinate set for the refined model of apo PDE5* is recorded in Table 4. The model contains 323 amino acid residues, 537-858 (residue Glu 681A has been numbered to maintain PDE5 numbering scheme), a zinc ion and a magnesium ion, as well as 222 water molecules.

### EXAMPLE 9

### Data Collection, Structure Determination and Refinement of the Soakable C2 Crystal Form of PDE5* Soaked with UK-088,800

The structure of the UK-088,800 soaked into the C2 crystal form of PDE5* was solved by difference Fourier methods.

X-ray diffraction data were collected with an RaxisIV image plate detector on an in-house FR-D rotating anode (Rigaku), with Osmic mirrors (MSC). All data were processed using the HKL package (Otwinowski & Minor, 1997). Data collection statistics are summarised in Table 1 (UK-088,800).

Refinement was carried out in the resolution range 30-1.5 Å using CNX (Brünger et al., 1998) with the “mlf” maximum likelihood target function. Partial structure factors from a flat bulk-solvent model and anisotropic B-factor correction were supplied throughout the refinement. The R-factor for the refined model is 0.219 (free R-factor, 5% of data, 0.228) for all data in the resolution range 30-1.5 Å. The refinement statistics are summarised in Table 2 (UK-088,800).

The co-ordinate set for the refined model of PDE5*-UK-088,800 is recorded in Table 5. The model contains 323 amino acid residues, 537-858 (residue Glu 681A has been numbered to maintain PDE5 numbering scheme), the ligand UK-088,800, a zinc ion and a magnesium ion, as well as 183 water molecules.

### EXAMPLE 10

### Comparison of the Apo PDE5* and PDE5* Soaked with UK-088,800

The (fo-fc)α_{calc} electron density map of PDE5* soaked with UK-088,800 calculated with experimental structure factor amplitudes and model phases derived prior to incorporation of the ligand into the model, shows a clearly featured peak that can unequivocally be interpreted as UK-088,880 (FIG. 3). In contrast, the equivalent (fo-fc)α_{calc} electron density map of apo PDE5* shows no significant residual features that could be interpreted as a small molecule ligand. The experiment allowed the experimental determination of the binding mode of UK-088,800 to PDE5* and demonstrates that the C2 crystal form of PDE5* as described herein is amenable to ligand soaking. The structure of PDE5* in the C2 crystal form does not show significant differences to that observed with the P2₁, crystal form.

TABLE 1
Data collection statistics for the soakable C2
crystal form of PDE5*

| | Apo | UK-088,800 |
|---|---|---|
| Resolution [Å] | 1.6 | 1.5 |
| Unique reflections | 41189 | 52038 |
| Completeness [%] | 94.5 | 99.5 |
| Redundancy | 2.9 | 3.5 |
| R_{sym}^{a} | 0.074 | 0.049 |

${R}_{sym}^{a} = {\sum }_{hkl} {\sum }_{i} \vert {I}_{i} ( hkl ) - \overline{I ( hkl )} \vert / {\sum }_{hkl} {\sum }_{i} \vert {I}_{i} ( hkl ) \vert$

TABLE 2
Refinement statistics of soakable C2 crystal form of PDE5*

| | Apo | UK-088,800 |
|---|---|---|
| Resolution range, Å | 30.0-1.6 | 30.0-1.5 |
| Amino acid residues, no. | 323 | 323 |
| Water molecules, no. | 222 | 183 |
| rmsd bond length, Å | 0.005 | 0.005 |
| rmsd bond angles, ° | 1.130 | 1.158 |
| ^{a}R-factor | 0.231 | 0.219 |
| Free R-factor (5% of data) | 0.267 | 0.228 |

${}^{a} R = {\sum }_{hkl} \vert \vert {F}_{obs} \vert - k \vert {F}_{calc} \vert \vert / {\sum }_{hkl} \vert {F}_{obs} \vert$

TABLE 3
Atomic coordinates for baculovirus - expressed PDE5* complex with Sildenafil
Atom Number, Atom Type, Residue Type, Residue Number Cartesian Coordinates
X, Y, Z, Atom Occupancy (O) and Temperature Factor (B)

| | | | | | | X | Y | Z | O | B |
|---|---|---|---|---|---|---|---|---|---|---|
| ATOM | 1 | CB | THR | A | 537 | −3.008 | 28.295 | 13.745 | 1.00 | 40.04 |
| ATOM | 2 | OG1 | THR | A | 537 | −4.423 | 28.364 | 13.527 | 1.00 | 40.20 |
| ATOM | 3 | CG2 | THR | A | 537 | −2.273 | 28.516 | 12.435 | 1.00 | 40.01 |
| ATOM | 4 | C | THR | A | 537 | −2.764 | 26.998 | 15.853 | 1.00 | 42.52 |
| ATOM | 5 | O | THR | A | 537 | −3.657 | 26.395 | 16.451 | 1.00 | 44.33 |
| ATOM | 6 | N | THR | A | 537 | −3.513 | 25.856 | 13.760 | 1.00 | 44.29 |
| ATOM | 7 | CA | THR | A | 537 | −2.652 | 26.924 | 14.338 | 1.00 | 43.47 |
| ATOM | 8 | N | ARG | A | 538 | −1.857 | 27.751 | 16.466 | 1.00 | 33.65 |
| ATOM | 9 | CA | ARG | A | 538 | −1.818 | 27.897 | 17.916 | 1.00 | 31.51 |
| ATOM | 10 | CB | ARG | A | 538 | −3.201 | 28.267 | 18.463 | 1.00 | 51.67 |
| ATOM | 11 | CG | ARG | A | 538 | −3.420 | 27.913 | 19.928 | 1.00 | 52.25 |
| ATOM | 12 | CD | ARG | A | 538 | −2.377 | 28.532 | 20.845 | 1.00 | 52.37 |
| ATOM | 13 | NE | ARG | A | 538 | −1.851 | 27.547 | 21.788 | 1.00 | 52.59 |
| ATOM | 14 | CZ | ARG | A | 538 | −1.048 | 27.833 | 22.809 | 1.00 | 52.58 |
| ATOM | 15 | NH1 | ARG | A | 538 | −0.676 | 29.087 | 23.032 | 1.00 | 52.27 |
| ATOM | 16 | NH2 | ARG | A | 538 | −0.608 | 26.864 | 23.600 | 1.00 | 52.33 |
| ATOM | 17 | C | ARG | A | 538 | −1.336 | 26.584 | 18.521 | 1.00 | 30.67 |
| ATOM | 18 | O | ARG | A | 538 | −0.175 | 26.465 | 18.906 | 1.00 | 30.03 |
| ATOM | 19 | N | GLU | A | 539 | −2.221 | 25.594 | 18.593 | 1.00 | 27.90 |
| ATOM | 20 | CA | GLU | A | 539 | −1.827 | 24.310 | 19.151 | 1.00 | 27.13 |
| ATOM | 21 | CB | GLU | A | 539 | −3.000 | 23.322 | 19.146 | 1.00 | 33.40 |
| ATOM | 22 | CG | GLU | A | 539 | −2.627 | 21.946 | 19.679 | 1.00 | 34.46 |
| ATOM | 23 | CD | GLU | A | 539 | −3.814 | 21.007 | 19.761 | 1.00 | 34.35 |
| ATOM | 24 | OE1 | GLU | A | 539 | −4.656 | 21.037 | 18.843 | 1.00 | 34.50 |
| ATOM | 25 | OE2 | GLU | A | 539 | −3.899 | 20.233 | 20.738 | 1.00 | 34.57 |
| ATOM | 26 | C | GLU | A | 539 | −0.666 | 23.734 | 18.346 | 1.00 | 27.02 |
| ATOM | 27 | O | GLU | A | 539 | 0.280 | 23.189 | 18.909 | 1.00 | 27.32 |
| ATOM | 28 | N | LEU | A | 540 | −0.739 | 23.866 | 17.027 | 1.00 | 26.77 |
| ATOM | 29 | CA | LEU | A | 540 | 0.320 | 23.354 | 16.166 | 1.00 | 26.36 |
| ATOM | 30 | CB | LEU | A | 540 | −0.070 | 23.496 | 14.695 | 1.00 | 24.87 |
| ATOM | 31 | CG | LEU | A | 540 | 1.027 | 23.155 | 13.682 | 1.00 | 24.86 |
| ATOM | 32 | CD1 | LEU | A | 540 | 1.426 | 21.689 | 13.833 | 1.00 | 25.12 |
| ATOM | 33 | CD2 | LEU | A | 540 | 0.531 | 23.447 | 12.264 | 1.00 | 25.63 |
| ATOM | 34 | C | LEU | A | 540 | 1.631 | 24.091 | 16.415 | 1.00 | 26.72 |
| ATOM | 35 | O | LEU | A | 540 | 2.681 | 23.479 | 16.603 | 1.00 | 26.47 |
| ATOM | 36 | N | GLN | A | 541 | 1.573 | 25.416 | 16.407 | 1.00 | 25.80 |
| ATOM | 37 | CA | GLN | A | 541 | 2.777 | 26.196 | 16.626 | 1.00 | 26.31 |
| ATOM | 38 | CB | GLN | A | 541 | 2.455 | 27.682 | 16.484 | 1.00 | 28.46 |
| ATOM | 39 | CG | GLN | A | 541 | 2.334 | 28.108 | 15.029 | 1.00 | 28.55 |
| ATOM | 40 | CD | GLN | A | 541 | 1.709 | 29.475 | 14.868 | 1.00 | 28.37 |
| ATOM | 41 | OE1 | GLN | A | 541 | 1.843 | 30.334 | 15.736 | 1.00 | 28.46 |
| ATOM | 42 | NE2 | GLN | A | 541 | 1.033 | 29.687 | 13.747 | 1.00 | 29.26 |
| ATOM | 43 | C | GLN | A | 541 | 3.415 | 25.894 | 17.978 | 1.00 | 25.96 |
| ATOM | 44 | O | GLN | A | 541 | 4.636 | 25.785 | 18.082 | 1.00 | 26.05 |
| ATOM | 45 | N | SER | A | 542 | 2.589 | 25.733 | 19.008 | 1.00 | 27.15 |
| ATOM | 46 | CA | SER | A | 542 | 3.103 | 25.440 | 20.341 | 1.00 | 27.52 |
| ATOM | 47 | CB | SER | A | 542 | 1.980 | 25.510 | 21.379 | 1.00 | 38.20 |
| ATOM | 48 | OG | SER | A | 542 | 1.535 | 26.841 | 21.558 | 1.00 | 38.87 |
| ATOM | 49 | C | SER | A | 542 | 3.752 | 24.067 | 20.390 | 1.00 | 27.50 |
| ATOM | 50 | O | SER | A | 542 | 4.795 | 23.888 | 21.015 | 1.00 | 27.75 |
| ATOM | 51 | N | LEU | A | 543 | 3.131 | 23.095 | 19.732 | 1.00 | 23.90 |
| ATOM | 52 | CA | LEU | A | 543 | 3.676 | 21.743 | 19.723 | 1.00 | 23.62 |
| ATOM | 53 | CB | LEU | A | 543 | 2.702 | 20.785 | 19.022 | 1.00 | 31.43 |
| ATOM | 54 | CG | LEU | A | 543 | 2.929 | 19.274 | 19.167 | 1.00 | 32.27 |
| ATOM | 55 | CD1 | LEU | A | 543 | 1.959 | 18.529 | 18.263 | 1.00 | 31.54 |
| ATOM | 56 | CD2 | LEU | A | 543 | 4.340 | 18.904 | 18.800 | 1.00 | 32.06 |
| ATOM | 57 | C | LEU | A | 543 | 5.025 | 21.725 | 19.009 | 1.00 | 23.65 |
| ATOM | 58 | O | LEU | A | 543 | 5.998 | 21.144 | 19.498 | 1.00 | 23.61 |
| ATOM | 59 | N | ALA | A | 544 | 5.079 | 22.365 | 17.848 | 1.00 | 24.41 |
| ATOM | 60 | CA | ALA | A | 544 | 6.295 | 22.402 | 17.052 | 1.00 | 24.73 |
| ATOM | 61 | CB | ALA | A | 544 | 6.014 | 23.090 | 15.728 | 1.00 | 23.31 |
| ATOM | 62 | C | ALA | A | 544 | 7.477 | 23.076 | 17.748 | 1.00 | 25.97 |
| ATOM | 63 | O | ALA | A | 544 | 8.619 | 22.642 | 17.600 | 1.00 | 25.19 |
| ATOM | 64 | N | ALA | A | 545 | 7.202 | 24.125 | 18.512 | 1.00 | 29.21 |
| ATOM | 65 | CA | ALA | A | 545 | 8.259 | 24.861 | 19.196 | 1.00 | 30.03 |
| ATOM | 66 | CB | ALA | A | 545 | 7.837 | 26.315 | 19.351 | 1.00 | 32.13 |
| ATOM | 67 | C | ALA | A | 545 | 8.660 | 24.288 | 20.553 | 1.00 | 30.21 |
| ATOM | 68 | O | ALA | A | 545 | 9.698 | 24.657 | 21.105 | 1.00 | 30.14 |
| ATOM | 69 | N | ALA | A | 546 | 7.849 | 23.382 | 21.088 | 1.00 | 28.91 |
| ATOM | 70 | CA | ALA | A | 546 | 8.126 | 22.790 | 22.391 | 1.00 | 28.92 |
| ATOM | 71 | CB | ALA | A | 546 | 6.878 | 22.068 | 22.913 | 1.00 | 27.14 |
| ATOM | 72 | C | ALA | A | 546 | 9.308 | 21.831 | 22.400 | 1.00 | 29.12 |
| ATOM | 73 | O | ALA | A | 546 | 9.648 | 21.222 | 21.381 | 1.00 | 29.57 |
| ATOM | 74 | N | VAL | A | 547 | 9.936 | 21.702 | 23.564 | 1.00 | 27.29 |
| ATOM | 75 | CA | VAL | A | 547 | 11.060 | 20.791 | 23.714 | 1.00 | 26.19 |
| ATOM | 76 | CB | VAL | A | 547 | 11.917 | 21.148 | 24.947 | 1.00 | 37.00 |
| ATOM | 77 | CG1 | VAL | A | 547 | 12.925 | 20.042 | 25.223 | 1.00 | 37.18 |
| ATOM | 78 | CG2 | VAL | A | 547 | 12.635 | 22.464 | 24.711 | 1.00 | 37.04 |
| ATOM | 79 | C | VAL | A | 547 | 10.482 | 19.395 | 23.903 | 1.00 | 26.09 |
| ATOM | 80 | O | VAL | A | 547 | 9.473 | 19.226 | 24.588 | 1.00 | 27.35 |
| ATOM | 81 | N | VAL | A | 548 | 11.110 | 18.400 | 23.286 | 1.00 | 28.09 |
| ATOM | 82 | CA | VAL | A | 548 | 10.638 | 17.029 | 23.409 | 1.00 | 28.48 |
| ATOM | 83 | CB | VAL | A | 548 | 10.792 | 16.254 | 22.079 | 1.00 | 23.53 |
| ATOM | 84 | CG1 | VAL | A | 548 | 10.198 | 14.845 | 22.215 | 1.00 | 24.59 |
| ATOM | 85 | CG2 | VAL | A | 548 | 10.123 | 17.016 | 20.953 | 1.00 | 23.63 |
| ATOM | 86 | C | VAL | A | 548 | 11.468 | 16.336 | 24.475 | 1.00 | 28.05 |
| ATOM | 87 | O | VAL | A | 548 | 12.637 | 16.033 | 24.252 | 1.00 | 29.14 |
| ATOM | 88 | N | PRO | A | 549 | 10.879 | 16.080 | 25.655 | 1.00 | 28.24 |
| ATOM | 89 | CD | PRO | A | 549 | 9.550 | 16.472 | 26.151 | 1.00 | 28.37 |
| ATOM | 90 | CA | PRO | A | 549 | 11.647 | 15.414 | 26.709 | 1.00 | 28.05 |
| ATOM | 91 | CB | PRO | A | 549 | 10.648 | 15.321 | 27.858 | 1.00 | 28.48 |
| ATOM | 92 | CG | PRO | A | 549 | 9.781 | 16.524 | 27.640 | 1.00 | 28.59 |
| ATOM | 93 | C | PRO | A | 549 | 12.133 | 14.045 | 26.254 | 1.00 | 28.74 |
| ATOM | 94 | O | PRO | A | 549 | 11.639 | 13.495 | 25.262 | 1.00 | 27.64 |
| ATOM | 95 | N | SER | A | 550 | 13.103 | 13.505 | 26.977 | 1.00 | 30.38 |
| ATOM | 96 | CA | SER | A | 550 | 13.658 | 12.202 | 26.649 | 1.00 | 30.47 |
| ATOM | 97 | CB | SER | A | 550 | 14.923 | 11.963 | 27.464 | 1.00 | 32.70 |
| ATOM | 98 | OG | SER | A | 550 | 14.611 | 11.959 | 28.843 | 1.00 | 32.28 |
| ATOM | 99 | C | SER | A | 550 | 12.654 | 11.101 | 26.955 | 1.00 | 29.91 |
| ATOM | 100 | O | SER | A | 550 | 11.683 | 11.310 | 27.684 | 1.00 | 30.19 |
| ATOM | 101 | N | ALA | A | 551 | 12.898 | 9.922 | 26.396 | 1.00 | 27.46 |
| ATOM | 102 | CA | ALA | A | 551 | 12.022 | 8.784 | 26.626 | 1.00 | 27.31 |
| ATOM | 103 | CB | ALA | A | 551 | 12.451 | 7.610 | 25.756 | 1.00 | 33.59 |
| ATOM | 104 | C | ALA | A | 551 | 12.064 | 8.389 | 28.097 | 1.00 | 27.99 |
| ATOM | 105 | O | ALA | A | 551 | 11.031 | 8.093 | 28.700 | 1.00 | 27.97 |
| ATOM | 106 | N | GLN | A | 552 | 13.264 | 8.382 | 28.670 | 1.00 | 30.15 |
| ATOM | 107 | CA | GLN | A | 552 | 13.433 | 8.019 | 30.074 | 1.00 | 31.28 |
| ATOM | 108 | CB | GLN | A | 552 | 14.917 | 8.099 | 30.457 | 1.00 | 50.35 |
| ATOM | 109 | CG | GLN | A | 552 | 15.547 | 9.470 | 30.254 | 1.00 | 51.01 |
| ATOM | 110 | CD | GLN | A | 552 | 17.068 | 9.439 | 30.310 | 1.00 | 51.35 |
| ATOM | 111 | OE1 | GLN | A | 552 | 17.725 | 8.932 | 29.398 | 1.00 | 51.08 |
| ATOM | 112 | NE2 | GLN | A | 552 | 17.635 | 9.979 | 31.385 | 1.00 | 51.12 |
| ATOM | 113 | C | GLN | A | 552 | 12.595 | 8.934 | 30.969 | 1.00 | 31.11 |
| ATOM | 114 | O | GLN | A | 552 | 11.888 | 8.470 | 31.868 | 1.00 | 30.93 |
| ATOM | 115 | N | THR | A | 553 | 12.660 | 10.232 | 30.704 | 1.00 | 32.81 |
| ATOM | 116 | CA | THR | A | 553 | 11.904 | 11.202 | 31.484 | 1.00 | 32.49 |
| ATOM | 117 | CB | THR | A | 553 | 12.250 | 12.637 | 31.060 | 1.00 | 35.77 |
| ATOM | 118 | OG1 | THR | A | 553 | 13.615 | 12.915 | 31.399 | 1.00 | 35.76 |
| ATOM | 119 | CG2 | THR | A | 553 | 11.339 | 13.639 | 31.761 | 1.00 | 35.80 |
| ATOM | 120 | C | THR | A | 553 | 10.399 | 10.999 | 31.333 | 1.00 | 32.89 |
| ATOM | 121 | O | THR | A | 553 | 9.658 | 11.065 | 32.313 | 1.00 | 33.04 |
| ATOM | 122 | N | LEU | A | 554 | 9.954 | 10.750 | 30.105 | 1.00 | 29.86 |
| ATOM | 123 | CA | LEU | A | 554 | 8.534 | 10.545 | 29.827 | 1.00 | 28.95 |
| ATOM | 124 | CB | LEU | A | 554 | 8.278 | 10.693 | 28.325 | 1.00 | 27.17 |
| ATOM | 125 | CG | LEU | A | 554 | 8.586 | 12.088 | 27.769 | 1.00 | 27.34 |
| ATOM | 126 | CD1 | LEU | A | 554 | 8.439 | 12.101 | 26.255 | 1.00 | 27.36 |
| ATOM | 127 | CD2 | LEU | A | 554 | 7.650 | 13.105 | 28.410 | 1.00 | 27.14 |
| ATOM | 128 | C | LEU | A | 554 | 8.011 | 9.197 | 30.326 | 1.00 | 28.80 |
| ATOM | 129 | O | LEU | A | 554 | 6.803 | 9.014 | 30.486 | 1.00 | 28.57 |
| ATOM | 130 | N | LYS | A | 555 | 8.925 | 8.260 | 30.561 | 1.00 | 24.74 |
| ATOM | 131 | CA | LYS | A | 555 | 8.588 | 6.935 | 31.071 | 1.00 | 25.34 |
| ATOM | 132 | CB | LYS | A | 555 | 7.764 | 7.072 | 32.356 | 1.00 | 44.27 |
| ATOM | 133 | CG | LYS | A | 555 | 8.420 | 7.960 | 33.402 | 1.00 | 45.10 |
| ATOM | 134 | CD | LYS | A | 555 | 7.451 | 8.363 | 34.501 | 1.00 | 45.29 |
| ATOM | 135 | CE | LYS | A | 555 | 8.106 | 9.352 | 35.455 | 1.00 | 45.10 |
| ATOM | 136 | NZ | LYS | A | 555 | 7.173 | 9.818 | 36.518 | 1.00 | 45.31 |
| ATOM | 137 | C | LYS | A | 555 | 7.818 | 6.084 | 30.060 | 1.00 | 24.58 |
| ATOM | 138 | O | LYS | A | 555 | 7.148 | 5.116 | 30.433 | 1.00 | 25.45 |
| ATOM | 139 | N | ILE | A | 556 | 7.932 | 6.430 | 28.783 | 1.00 | 26.12 |
| ATOM | 140 | CA | ILE | A | 556 | 7.222 | 5.699 | 27.740 | 1.00 | 26.45 |
| ATOM | 141 | CB | ILE | A | 556 | 7.160 | 6.515 | 26.432 | 1.00 | 24.50 |
| ATOM | 142 | CG2 | ILE | A | 556 | 6.294 | 7.753 | 26.640 | 1.00 | 24.65 |
| ATOM | 143 | CG1 | ILE | A | 556 | 8.571 | 6.907 | 25.987 | 1.00 | 24.30 |
| ATOM | 144 | CD1 | ILE | A | 556 | 8.631 | 7.477 | 24.582 | 1.00 | 24.18 |
| ATOM | 145 | C | ILE | A | 556 | 7.806 | 4.321 | 27.446 | 1.00 | 26.74 |
| ATOM | 146 | O | ILE | A | 556 | 7.203 | 3.525 | 26.722 | 1.00 | 26.46 |
| ATOM | 147 | N | THR | A | 557 | 8.974 | 4.027 | 28.004 | 1.00 | 26.58 |
| ATOM | 148 | CA | THR | A | 557 | 9.571 | 2.718 | 27.782 | 1.00 | 26.91 |
| ATOM | 149 | CB | THR | A | 557 | 11.098 | 2.750 | 27.964 | 1.00 | 33.52 |
| ATOM | 150 | OG1 | THR | A | 557 | 11.653 | 3.811 | 27.179 | 1.00 | 33.53 |
| ATOM | 151 | CG2 | THR | A | 557 | 11.709 | 1.430 | 27.506 | 1.00 | 33.78 |
| ATOM | 152 | C | THR | A | 557 | 8.983 | 1.711 | 28.766 | 1.00 | 26.91 |
| ATOM | 153 | O | THR | A | 557 | 9.108 | 0.501 | 28.578 | 1.00 | 27.08 |
| ATOM | 154 | N | ASP | A | 558 | 8.327 | 2.213 | 29.810 | 1.00 | 22.97 |
| ATOM | 155 | CA | ASP | A | 558 | 7.739 | 1.347 | 30.824 | 1.00 | 24.55 |
| ATOM | 156 | CB | ASP | A | 558 | 7.564 | 2.101 | 32.145 | 1.00 | 42.16 |
| ATOM | 157 | CG | ASP | A | 558 | 8.816 | 2.834 | 32.573 | 1.00 | 43.08 |
| ATOM | 158 | OD1 | ASP | A | 558 | 9.918 | 2.258 | 32.459 | 1.00 | 43.16 |
| ATOM | 159 | OD2 | ASP | A | 558 | 8.692 | 3.987 | 33.036 | 1.00 | 43.34 |
| ATOM | 160 | C | ASP | A | 558 | 6.386 | 0.797 | 30.408 | 1.00 | 23.87 |
| ATOM | 161 | O | ASP | A | 558 | 5.522 | 1.537 | 29.940 | 1.00 | 24.35 |
| ATOM | 162 | N | PHE | A | 559 | 6.200 | −0.505 | 30.593 | 1.00 | 26.69 |
| ATOM | 163 | CA | PHE | A | 559 | 4.936 | −1.140 | 30.257 | 1.00 | 26.24 |
| ATOM | 164 | CB | PHE | A | 559 | 5.052 | −2.662 | 30.372 | 1.00 | 34.04 |
| ATOM | 165 | CG | PHE | A | 559 | 5.885 | −3.290 | 29.292 | 1.00 | 34.73 |
| ATOM | 166 | CD1 | PHE | A | 559 | 5.537 | −3.135 | 27.952 | 1.00 | 34.65 |
| ATOM | 167 | CD2 | PHE | A | 559 | 7.018 | −4.036 | 29.610 | 1.00 | 34.64 |
| ATOM | 168 | CE1 | PHE | A | 559 | 6.304 | −3.713 | 26.943 | 1.00 | 34.43 |
| ATOM | 169 | CE2 | PHE | A | 559 | 7.793 | −4.618 | 28.610 | 1.00 | 34.92 |
| ATOM | 170 | CZ | PHE | A | 559 | 7.435 | −4.456 | 27.272 | 1.00 | 34.80 |
| ATOM | 171 | C | PHE | A | 559 | 3.841 | −0.639 | 31.196 | 1.00 | 26.88 |
| ATOM | 172 | O | PHE | A | 559 | 2.664 | −0.631 | 30.837 | 1.00 | 25.97 |
| ATOM | 173 | N | SER | A | 560 | 4.245 | −0.213 | 32.389 | 1.00 | 25.49 |
| ATOM | 174 | CA | SER | A | 560 | 3.321 | 0.285 | 33.407 | 1.00 | 26.93 |
| ATOM | 175 | CB | SER | A | 560 | 3.945 | 0.120 | 34.797 | 1.00 | 39.67 |
| ATOM | 176 | OG | SER | A | 560 | 5.170 | 0.830 | 34.899 | 1.00 | 40.02 |
| ATOM | 177 | C | SER | A | 560 | 2.920 | 1.745 | 33.197 | 1.00 | 26.06 |
| ATOM | 178 | O | SER | A | 560 | 2.205 | 2.327 | 34.018 | 1.00 | 26.04 |
| ATOM | 179 | N | PHE | A | 561 | 3.382 | 2.326 | 32.098 | 1.00 | 26.05 |
| ATOM | 180 | CA | PHE | A | 561 | 3.081 | 3.711 | 31.751 | 1.00 | 25.67 |
| ATOM | 181 | CB | PHE | A | 561 | 3.551 | 3.982 | 30.321 | 1.00 | 26.66 |
| ATOM | 182 | CG | PHE | A | 561 | 3.158 | 5.330 | 29.795 | 1.00 | 25.48 |
| ATOM | 183 | CD1 | PHE | A | 561 | 3.990 | 6.430 | 29.972 | 1.00 | 26.67 |
| ATOM | 184 | CD2 | PHE | A | 561 | 1.948 | 5.502 | 29.127 | 1.00 | 26.67 |
| ATOM | 185 | CE1 | PHE | A | 561 | 3.624 | 7.686 | 29.489 | 1.00 | 26.12 |
| ATOM | 186 | CE2 | PHE | A | 561 | 1.571 | 6.748 | 28.642 | 1.00 | 25.76 |
| ATOM | 187 | CZ | PHE | A | 561 | 2.412 | 7.844 | 28.823 | 1.00 | 26.46 |
| ATOM | 188 | C | PHE | A | 561 | 1.597 | 4.082 | 31.865 | 1.00 | 26.23 |
| ATOM | 189 | O | PHE | A | 561 | 0.715 | 3.293 | 31.533 | 1.00 | 25.55 |
| ATOM | 190 | N | SER | A | 562 | 1.335 | 5.304 | 32.320 | 1.00 | 24.91 |
| ATOM | 191 | CA | SER | A | 562 | −0.029 | 5.803 | 32.463 | 1.00 | 26.24 |
| ATOM | 192 | CB | SER | A | 562 | −0.406 | 5.906 | 33.940 | 1.00 | 42.72 |
| ATOM | 193 | OG | SER | A | 562 | −1.720 | 6.414 | 34.084 | 1.00 | 43.29 |
| ATOM | 194 | C | SER | A | 562 | −0.134 | 7.179 | 31.817 | 1.00 | 24.23 |
| ATOM | 195 | O | SER | A | 562 | 0.730 | 8.024 | 32.013 | 1.00 | 25.59 |
| ATOM | 196 | N | ASP | A | 563 | −1.199 | 7.401 | 31.055 | 1.00 | 23.00 |
| ATOM | 197 | CA | ASP | A | 563 | −1.410 | 8.675 | 30.373 | 1.00 | 22.76 |
| ATOM | 198 | CB | ASP | A | 563 | −2.135 | 8.446 | 29.043 | 1.00 | 25.24 |
| ATOM | 199 | CG | ASP | A | 563 | −3.635 | 8.228 | 29.230 | 1.00 | 23.77 |
| ATOM | 200 | OD1 | ASP | A | 563 | −4.037 | 7.150 | 29.718 | 1.00 | 23.35 |
| ATOM | 201 | OD2 | ASP | A | 563 | −4.421 | 9.143 | 28.906 | 1.00 | 24.81 |
| ATOM | 202 | C | ASP | A | 563 | −2.263 | 9.659 | 31.176 | 1.00 | 22.96 |
| ATOM | 203 | O | ASP | A | 563 | −2.297 | 10.848 | 30.865 | 1.00 | 21.79 |
| ATOM | 204 | N | PHE | A | 564 | −2.958 | 9.150 | 32.187 | 1.00 | 24.29 |
| ATOM | 205 | CA | PHE | A | 564 | −3.878 | 9.962 | 32.987 | 1.00 | 25.23 |
| ATOM | 206 | CB | PHE | A | 564 | −4.394 | 9.157 | 34.180 | 1.00 | 41.24 |
| ATOM | 207 | CG | PHE | A | 564 | −5.576 | 9.787 | 34.857 | 1.00 | 41.58 |
| ATOM | 208 | CD1 | PHE | A | 564 | −6.793 | 9.895 | 34.191 | 1.00 | 41.99 |
| ATOM | 209 | CD2 | PHE | A | 564 | −5.469 | 10.300 | 36.146 | 1.00 | 42.28 |
| ATOM | 210 | CE1 | PHE | A | 564 | −7.888 | 10.506 | 34.797 | 1.00 | 41.91 |
| ATOM | 211 | CE2 | PHE | A | 564 | −6.558 | 10.915 | 36.763 | 1.00 | 41.92 |
| ATOM | 212 | CZ | PHE | A | 564 | −7.769 | 11.018 | 36.086 | 1.00 | 41.83 |
| ATOM | 213 | C | PHE | A | 564 | −3.410 | 11.321 | 33.490 | 1.00 | 25.10 |
| ATOM | 214 | O | PHE | A | 564 | −4.185 | 12.278 | 33.488 | 1.00 | 25.27 |
| ATOM | 215 | N | GLU | A | 565 | −2.158 | 11.406 | 33.921 | 1.00 | 29.45 |
| ATOM | 216 | CA | GLU | A | 565 | −1.618 | 12.651 | 34.459 | 1.00 | 30.36 |
| ATOM | 217 | CB | GLU | A | 565 | −0.456 | 12.341 | 35.408 | 1.00 | 40.02 |
| ATOM | 218 | CG | GLU | A | 565 | −0.821 | 11.524 | 36.645 | 1.00 | 40.24 |
| ATOM | 219 | CD | GLU | A | 565 | −1.312 | 10.120 | 36.320 | 1.00 | 40.61 |
| ATOM | 220 | OE1 | GLU | A | 565 | −0.655 | 9.424 | 35.514 | 1.00 | 40.05 |
| ATOM | 221 | OE2 | GLU | A | 565 | −2.352 | 9.711 | 36.883 | 1.00 | 40.18 |
| ATOM | 222 | C | GLU | A | 565 | −1.144 | 13.652 | 33.409 | 1.00 | 30.08 |
| ATOM | 223 | O | GLU | A | 565 | −0.860 | 14.810 | 33.732 | 1.00 | 30.87 |
| ATOM | 224 | N | LEU | A | 566 | −1.070 | 13.217 | 32.155 | 1.00 | 25.91 |
| ATOM | 225 | CA | LEU | A | 566 | −0.587 | 14.073 | 31.078 | 1.00 | 25.51 |
| ATOM | 226 | CB | LEU | A | 566 | 0.157 | 13.219 | 30.051 | 1.00 | 30.68 |
| ATOM | 227 | CG | LEU | A | 566 | 1.256 | 12.294 | 30.578 | 1.00 | 31.45 |
| ATOM | 228 | CD1 | LEU | A | 566 | 1.775 | 11.429 | 29.437 | 1.00 | 30.44 |
| ATOM | 229 | CD2 | LEU | A | 566 | 2.377 | 13.112 | 31.186 | 1.00 | 31.07 |
| ATOM | 230 | C | LEU | A | 566 | −1.642 | 14.889 | 30.349 | 1.00 | 25.31 |
| ATOM | 231 | O | LEU | A | 566 | −2.793 | 14.475 | 30.220 | 1.00 | 24.57 |
| ATOM | 232 | N | SER | A | 567 | −1.228 | 16.054 | 29.859 | 1.00 | 27.06 |
| ATOM | 233 | CA | SER | A | 567 | −2.110 | 16.928 | 29.096 | 1.00 | 26.87 |
| ATOM | 234 | CB | SER | A | 567 | −1.619 | 18.373 | 29.159 | 1.00 | 26.86 |
| ATOM | 235 | OG | SER | A | 567 | −0.406 | 18.519 | 28.439 | 1.00 | 26.48 |
| ATOM | 236 | C | SER | A | 567 | −2.038 | 16.447 | 27.650 | 1.00 | 26.54 |
| ATOM | 237 | O | SER | A | 567 | −1.187 | 15.617 | 27.314 | 1.00 | 25.78 |
| ATOM | 238 | N | ASP | A | 568 | −2.921 | 16.963 | 26.800 | 1.00 | 25.65 |
| ATOM | 239 | CA | ASP | A | 568 | −2.935 | 16.588 | 25.388 | 1.00 | 25.44 |
| ATOM | 240 | CB | ASP | A | 568 | −4.077 | 17.292 | 24.657 | 1.00 | 35.03 |
| ATOM | 241 | CG | ASP | A | 568 | −5.437 | 16.732 | 25.028 | 1.00 | 35.82 |
| ATOM | 242 | OD1 | ASP | A | 568 | −6.450 | 17.284 | 24.555 | 1.00 | 36.02 |
| ATOM | 243 | OD2 | ASP | A | 568 | −5.489 | 15.744 | 25.788 | 1.00 | 35.24 |
| ATOM | 244 | C | ASP | A | 568 | −1.604 | 16.946 | 24.739 | 1.00 | 26.02 |
| ATOM | 245 | O | ASP | A | 568 | −1.040 | 16.154 | 23.981 | 1.00 | 24.45 |
| ATOM | 246 | N | LEU | A | 569 | −1.104 | 18.140 | 25.040 | 1.00 | 26.56 |
| ATOM | 247 | CA | LEU | A | 569 | 0.177 | 18.584 | 24.501 | 1.00 | 27.05 |
| ATOM | 248 | CB | LEU | A | 569 | 0.544 | 19.958 | 25.066 | 1.00 | 35.85 |
| ATOM | 249 | CG | LEU | A | 569 | 1.958 | 20.460 | 24.751 | 1.00 | 36.55 |
| ATOM | 250 | CD1 | LEU | A | 569 | 2.110 | 20.683 | 23.255 | 1.00 | 36.24 |
| ATOM | 251 | CD2 | LEU | A | 569 | 2.211 | 21.755 | 25.509 | 1.00 | 36.44 |
| ATOM | 252 | C | LEU | A | 569 | 1.267 | 17.582 | 24.864 | 1.00 | 25.81 |
| ATOM | 253 | O | LEU | A | 569 | 2.080 | 17.214 | 24.021 | 1.00 | 27.30 |
| ATOM | 254 | N | GLU | A | 570 | 1.283 | 17.151 | 26.126 | 1.00 | 24.45 |
| ATOM | 255 | CA | GLU | A | 570 | 2.266 | 16.183 | 26.596 | 1.00 | 23.61 |
| ATOM | 256 | CB | GLU | A | 570 | 2.143 | 15.984 | 28.104 | 1.00 | 28.33 |
| ATOM | 257 | CG | GLU | A | 570 | 2.600 | 17.182 | 28.912 | 1.00 | 28.49 |
| ATOM | 258 | CD | GLU | A | 570 | 2.473 | 16.953 | 30.402 | 1.00 | 28.93 |
| ATOM | 259 | OE1 | GLU | A | 570 | 1.346 | 16.679 | 30.864 | 1.00 | 27.96 |
| ATOM | 260 | OE2 | GLU | A | 570 | 3.500 | 17.049 | 31.111 | 1.00 | 29.89 |
| ATOM | 261 | C | GLU | A | 570 | 2.149 | 14.832 | 25.892 | 1.00 | 23.15 |
| ATOM | 262 | O | GLU | A | 570 | 3.166 | 14.194 | 25.622 | 1.00 | 23.63 |
| ATOM | 263 | N | THR | A | 571 | 0.931 | 14.382 | 25.592 | 1.00 | 21.78 |
| ATOM | 264 | CA | THR | A | 571 | 0.803 | 13.099 | 24.897 | 1.00 | 21.61 |
| ATOM | 265 | CB | THR | A | 571 | −0.670 | 12.597 | 24.814 | 1.00 | 20.96 |
| ATOM | 266 | OG1 | THR | A | 571 | −1.477 | 13.550 | 24.117 | 1.00 | 22.41 |
| ATOM | 267 | CG2 | THR | A | 571 | −1.242 | 12.365 | 26.212 | 1.00 | 20.91 |
| ATOM | 268 | C | THR | A | 571 | 1.362 | 13.236 | 23.483 | 1.00 | 22.39 |
| ATOM | 269 | O | THR | A | 571 | 1.924 | 12.283 | 22.929 | 1.00 | 21.33 |
| ATOM | 270 | N | ALA | A | 572 | 1.218 | 14.425 | 22.906 | 1.00 | 21.89 |
| ATOM | 271 | CA | ALA | A | 572 | 1.726 | 14.683 | 21.564 | 1.00 | 22.51 |
| ATOM | 272 | CB | ALA | A | 572 | 1.235 | 16.037 | 21.061 | 1.00 | 23.95 |
| ATOM | 273 | C | ALA | A | 572 | 3.247 | 14.647 | 21.603 | 1.00 | 23.15 |
| ATOM | 274 | O | ALA | A | 572 | 3.885 | 14.120 | 20.690 | 1.00 | 23.62 |
| ATOM | 275 | N | LEU | A | 573 | 3.830 | 15.211 | 22.660 | 1.00 | 24.60 |
| ATOM | 276 | CA | LEU | A | 573 | 5.281 | 15.215 | 22.805 | 1.00 | 25.21 |
| ATOM | 277 | CB | LEU | A | 573 | 5.694 | 16.061 | 24.013 | 1.00 | 27.78 |
| ATOM | 278 | CG | LEU | A | 573 | 5.470 | 17.565 | 23.808 | 1.00 | 27.76 |
| ATOM | 279 | CD1 | LEU | A | 573 | 5.732 | 18.313 | 25.101 | 1.00 | 27.67 |
| ATOM | 280 | CD2 | LEU | A | 573 | 6.391 | 18.074 | 22.699 | 1.00 | 27.52 |
| ATOM | 281 | C | LEU | A | 573 | 5.777 | 13.784 | 22.953 | 1.00 | 25.21 |
| ATOM | 282 | O | LEU | A | 573 | 6.797 | 13.413 | 22.374 | 1.00 | 25.36 |
| ATOM | 283 | N | CYS | A | 574 | 5.052 | 12.979 | 23.725 | 1.00 | 21.84 |
| ATOM | 284 | CA | CYS | A | 574 | 5.432 | 11.581 | 23.897 | 1.00 | 21.46 |
| ATOM | 285 | CB | CYS | A | 574 | 4.446 | 10.852 | 24.807 | 1.00 | 23.13 |
| ATOM | 286 | SG | CYS | A | 574 | 4.663 | 11.173 | 26.560 | 1.00 | 22.80 |
| ATOM | 287 | C | CYS | A | 574 | 5.426 | 10.890 | 22.540 | 1.00 | 21.90 |
| ATOM | 288 | O | CYS | A | 574 | 6.277 | 10.047 | 22.260 | 1.00 | 21.49 |
| ATOM | 289 | N | THR | A | 575 | 4.449 | 11.240 | 21.705 | 1.00 | 23.44 |
| ATOM | 290 | CA | THR | A | 575 | 4.334 | 10.631 | 20.384 | 1.00 | 21.88 |
| ATOM | 291 | CB | THR | A | 575 | 3.006 | 11.042 | 19.699 | 1.00 | 21.68 |
| ATOM | 292 | OG1 | THR | A | 575 | 1.916 | 10.641 | 20.536 | 1.00 | 22.63 |
| ATOM | 293 | CG2 | THR | A | 575 | 2.857 | 10.370 | 18.340 | 1.00 | 22.72 |
| ATOM | 294 | C | THR | A | 575 | 5.534 | 11.000 | 19.518 | 1.00 | 22.93 |
| ATOM | 295 | O | THR | A | 575 | 6.111 | 10.140 | 18.865 | 1.00 | 20.91 |
| ATOM | 296 | N | ILE | A | 576 | 5.929 | 12.268 | 19.529 | 1.00 | 22.16 |
| ATOM | 297 | CA | ILE | A | 576 | 7.091 | 12.673 | 18.748 | 1.00 | 23.75 |
| ATOM | 298 | CB | ILE | A | 576 | 7.403 | 14.171 | 18.924 | 1.00 | 26.26 |
| ATOM | 299 | CG2 | ILE | A | 576 | 8.756 | 14.495 | 18.311 | 1.00 | 26.23 |
| ATOM | 300 | CG1 | ILE | A | 576 | 6.318 | 15.016 | 18.260 | 1.00 | 26.29 |
| ATOM | 301 | CD1 | ILE | A | 576 | 6.476 | 16.494 | 18.490 | 1.00 | 26.83 |
| ATOM | 302 | C | ILE | A | 576 | 8.311 | 11.872 | 19.209 | 1.00 | 22.92 |
| ATOM | 303 | O | ILE | A | 576 | 9.111 | 11.416 | 18.394 | 1.00 | 23.77 |
| ATOM | 304 | N | ARG | A | 577 | 8.447 | 11.703 | 20.521 | 1.00 | 24.95 |
| ATOM | 305 | CA | ARG | A | 577 | 9.575 | 10.965 | 21.077 | 1.00 | 24.47 |
| ATOM | 306 | CB | ARG | A | 577 | 9.560 | 11.050 | 22.609 | 1.00 | 26.95 |
| ATOM | 307 | CG | ARG | A | 577 | 10.652 | 10.234 | 23.294 | 1.00 | 27.15 |
| ATOM | 308 | CD | ARG | A | 577 | 12.031 | 10.561 | 22.731 | 1.00 | 27.06 |
| ATOM | 309 | NE | ARG | A | 577 | 12.398 | 11.956 | 22.957 | 1.00 | 26.65 |
| ATOM | 310 | CZ | ARG | A | 577 | 13.447 | 12.556 | 22.401 | 1.00 | 27.38 |
| ATOM | 311 | NH1 | ARG | A | 577 | 14.247 | 11.888 | 21.578 | 1.00 | 27.39 |
| ATOM | 312 | NH2 | ARG | A | 577 | 13.696 | 13.829 | 22.666 | 1.00 | 26.81 |
| ATOM | 313 | C | ARG | A | 577 | 9.565 | 9.509 | 20.625 | 1.00 | 24.18 |
| ATOM | 314 | O | ARG | A | 577 | 10.619 | 8.913 | 20.416 | 1.00 | 24.97 |
| ATOM | 315 | N | MET | A | 578 | 8.372 | 8.938 | 20.471 | 1.00 | 21.76 |
| ATOM | 316 | CA | MET | A | 578 | 8.247 | 7.552 | 20.032 | 1.00 | 21.44 |
| ATOM | 317 | CB | MET | A | 578 | 6.782 | 7.122 | 20.042 | 1.00 | 25.13 |
| ATOM | 318 | CG | MET | A | 578 | 6.222 | 6.855 | 21.425 | 1.00 | 23.93 |
| ATOM | 319 | SD | MET | A | 578 | 4.483 | 6.363 | 21.351 | 1.00 | 23.95 |
| ATOM | 320 | CE | MET | A | 578 | 3.892 | 6.945 | 22.915 | 1.00 | 24.88 |
| ATOM | 321 | C | MET | A | 578 | 8.820 | 7.370 | 18.630 | 1.00 | 22.27 |
| ATOM | 322 | O | MET | A | 578 | 9.562 | 6.422 | 18.373 | 1.00 | 22.21 |
| ATOM | 323 | N | PHE | A | 579 | 8.462 | 8.270 | 17.722 | 1.00 | 22.29 |
| ATOM | 324 | CA | PHE | A | 579 | 8.960 | 8.194 | 16.355 | 1.00 | 22.58 |
| ATOM | 325 | CB | PHE | A | 579 | 8.317 | 9.267 | 15.474 | 1.00 | 22.36 |
| ATOM | 326 | CG | PHE | A | 579 | 6.970 | 8.885 | 14.925 | 1.00 | 22.77 |
| ATOM | 327 | CD1 | PHE | A | 579 | 5.812 | 9.077 | 15.672 | 1.00 | 22.73 |
| ATOM | 328 | CD2 | PHE | A | 579 | 6.864 | 8.341 | 13.649 | 1.00 | 21.91 |
| ATOM | 329 | CE1 | PHE | A | 579 | 4.560 | 8.733 | 15.151 | 1.00 | 22.92 |
| ATOM | 330 | CE2 | PHE | A | 579 | 5.625 | 7.993 | 13.119 | 1.00 | 22.30 |
| ATOM | 331 | CZ | PHE | A | 579 | 4.469 | 8.190 | 13.871 | 1.00 | 22.31 |
| ATOM | 332 | C | PHE | A | 579 | 10.466 | 8.390 | 16.328 | 1.00 | 24.14 |
| ATOM | 333 | O | PHE | A | 579 | 11.183 | 7.701 | 15.600 | 1.00 | 22.99 |
| ATOM | 334 | N | THR | A | 580 | 10.935 | 9.336 | 17.133 | 1.00 | 24.07 |
| ATOM | 335 | CA | THR | A | 580 | 12.350 | 9.667 | 17.198 | 1.00 | 24.45 |
| ATOM | 336 | CB | THR | A | 580 | 12.567 | 10.901 | 18.085 | 1.00 | 24.15 |
| ATOM | 337 | OG1 | THR | A | 580 | 11.712 | 11.961 | 17.636 | 1.00 | 24.88 |
| ATOM | 338 | OG2 | THR | A | 580 | 14.008 | 11.370 | 18.009 | 1.00 | 24.18 |
| ATOM | 339 | C | THR | A | 580 | 13.239 | 8.535 | 17.691 | 1.00 | 24.89 |
| ATOM | 340 | O | THR | A | 580 | 14.218 | 8.188 | 17.030 | 1.00 | 24.15 |
| ATOM | 341 | N | ASP | A | 581 | 12.903 | 7.955 | 18.840 | 1.00 | 26.03 |
| ATOM | 342 | CA | ASP | A | 581 | 13.703 | 6.870 | 19.402 | 1.00 | 26.27 |
| ATOM | 343 | CB | ASP | A | 581 | 13.395 | 6.710 | 20.894 | 1.00 | 26.96 |
| ATOM | 344 | CG | ASP | A | 581 | 14.029 | 7.808 | 21.733 | 1.00 | 26.96 |
| ATOM | 345 | OD1 | ASP | A | 581 | 14.376 | 8.868 | 21.167 | 1.00 | 26.78 |
| ATOM | 346 | OD2 | ASP | A | 581 | 14.176 | 7.620 | 22.958 | 1.00 | 27.14 |
| ATOM | 347 | C | ASP | A | 581 | 13.578 | 5.534 | 18.676 | 1.00 | 26.95 |
| ATOM | 348 | O | ASP | A | 581 | 14.306 | 4.589 | 18.978 | 1.00 | 27.55 |
| ATOM | 349 | N | LEU | A | 582 | 12.654 | 5.447 | 17.727 | 1.00 | 25.40 |
| ATOM | 350 | CA | LEU | A | 582 | 12.516 | 4.231 | 16.934 | 1.00 | 25.74 |
| ATOM | 351 | CB | LEU | A | 582 | 11.040 | 3.918 | 16.642 | 1.00 | 23.64 |
| ATOM | 352 | CG | LEU | A | 582 | 10.286 | 3.274 | 17.817 | 1.00 | 22.73 |
| ATOM | 353 | CD1 | LEU | A | 582 | 8.801 | 3.146 | 17.484 | 1.00 | 21.47 |
| ATOM | 354 | CD2 | LEU | A | 582 | 10.873 | 1.900 | 18.125 | 1.00 | 22.27 |
| ATOM | 355 | C | LEU | A | 582 | 13.284 | 4.483 | 15.635 | 1.00 | 26.83 |
| ATOM | 356 | O | LEU | A | 582 | 13.259 | 3.669 | 14.710 | 1.00 | 26.36 |
| ATOM | 357 | N | ASN | A | 583 | 13.967 | 5.625 | 15.588 | 1.00 | 30.93 |
| ATOM | 358 | CA | ASN | A | 583 | 14.767 | 6.026 | 14.433 | 1.00 | 32.87 |
| ATOM | 359 | CB | ASN | A | 583 | 15.944 | 5.065 | 14.247 | 1.00 | 52.34 |
| ATOM | 360 | CG | ASN | A | 583 | 17.069 | 5.331 | 15.225 | 1.00 | 53.08 |
| ATOM | 361 | OD1 | ASN | A | 583 | 18.026 | 4.561 | 15.312 | 1.00 | 53.41 |
| ATOM | 362 | ND2 | ASN | A | 583 | 16.967 | 6.432 | 15.962 | 1.00 | 53.31 |
| ATOM | 363 | C | ASN | A | 583 | 13.973 | 6.114 | 13.143 | 1.00 | 31.76 |
| ATOM | 364 | O | ASN | A | 583 | 14.443 | 5.706 | 12.085 | 1.00 | 32.63 |
| ATOM | 365 | N | LEU | A | 584 | 12.769 | 6.665 | 13.235 | 1.00 | 27.18 |
| ATOM | 366 | CA | LEU | A | 584 | 11.904 | 6.813 | 12.073 | 1.00 | 27.03 |
| ATOM | 367 | CB | LEU | A | 584 | 10.455 | 6.521 | 12.468 | 1.00 | 25.42 |
| ATOM | 368 | CG | LEU | A | 584 | 9.877 | 5.107 | 12.377 | 1.00 | 27.73 |
| ATOM | 369 | CD1 | LEU | A | 584 | 10.936 | 4.047 | 12.537 | 1.00 | 26.75 |
| ATOM | 370 | CD2 | LEU | A | 584 | 8.771 | 4.975 | 13.422 | 1.00 | 25.52 |
| ATOM | 371 | C | LEU | A | 584 | 11.971 | 8.208 | 11.468 | 1.00 | 26.54 |
| ATOM | 372 | O | LEU | A | 584 | 11.760 | 8.380 | 10.270 | 1.00 | 27.46 |
| ATOM | 373 | N | VAL | A | 585 | 12.265 | 9.204 | 12.296 | 1.00 | 28.54 |
| ATOM | 374 | CA | VAL | A | 585 | 12.300 | 10.589 | 11.831 | 1.00 | 29.02 |
| ATOM | 375 | CB | VAL | A | 585 | 12.403 | 11.550 | 13.025 | 1.00 | 27.38 |
| ATOM | 376 | CG1 | VAL | A | 585 | 12.404 | 12.993 | 12.545 | 1.00 | 27.75 |
| ATOM | 377 | CG2 | VAL | A | 585 | 11.236 | 11.310 | 13.971 | 1.00 | 27.59 |
| ATOM | 378 | C | VAL | A | 585 | 13.375 | 10.951 | 10.804 | 1.00 | 30.26 |
| ATOM | 379 | O | VAL | A | 585 | 13.059 | 11.379 | 9.693 | 1.00 | 29.85 |
| ATOM | 380 | N | GLN | A | 586 | 14.639 | 10.799 | 11.167 | 1.00 | 36.79 |
| ATOM | 381 | CA | GLN | A | 586 | 15.697 | 11.132 | 10.228 | 1.00 | 38.86 |
| ATOM | 382 | CB | GLN | A | 586 | 17.024 | 11.300 | 10.969 | 1.00 | 56.08 |
| ATOM | 383 | CG | GLN | A | 586 | 16.935 | 12.315 | 12.102 | 1.00 | 56.46 |
| ATOM | 384 | CD | GLN | A | 586 | 18.288 | 12.833 | 12.551 | 1.00 | 56.85 |
| ATOM | 385 | OE1 | GLN | A | 586 | 18.393 | 13.538 | 13.556 | 1.00 | 56.83 |
| ATOM | 386 | NE2 | GLN | A | 586 | 19.332 | 12.494 | 11.801 | 1.00 | 56.71 |
| ATOM | 387 | C | GLN | A | 586 | 15.796 | 10.046 | 9.163 | 1.00 | 38.13 |
| ATOM | 388 | O | GLN | A | 586 | 16.005 | 10.336 | 7.985 | 1.00 | 38.63 |
| ATOM | 389 | N | ASN | A | 587 | 15.615 | 8.799 | 9.586 | 1.00 | 34.73 |
| ATOM | 390 | CA | ASN | A | 587 | 15.665 | 7.652 | 8.685 | 1.00 | 34.34 |
| ATOM | 391 | CB | ASN | A | 587 | 15.120 | 6.420 | 9.399 | 1.00 | 37.16 |
| ATOM | 392 | CG | ASN | A | 587 | 15.340 | 5.140 | 8.620 | 1.00 | 36.75 |
| ATOM | 393 | OD1 | ASN | A | 587 | 15.392 | 5.141 | 7.389 | 1.00 | 36.63 |
| ATOM | 394 | ND2 | ASN | A | 587 | 15.450 | 4.031 | 9.341 | 1.00 | 37.09 |
| ATOM | 395 | C | ASN | A | 587 | 14.834 | 7.911 | 7.430 | 1.00 | 34.66 |
| ATOM | 396 | O | ASN | A | 587 | 15.294 | 7.691 | 6.307 | 1.00 | 34.46 |
| ATOM | 397 | N | PHE | A | 588 | 13.600 | 8.372 | 7.628 | 1.00 | 30.54 |
| ATOM | 398 | CA | PHE | A | 588 | 12.711 | 8.654 | 6.511 | 1.00 | 30.38 |
| ATOM | 399 | CB | PHE | A | 588 | 11.366 | 7.964 | 6.734 | 1.00 | 24.15 |
| ATOM | 400 | CG | PHE | A | 588 | 11.487 | 6.479 | 6.910 | 1.00 | 23.77 |
| ATOM | 401 | CD1 | PHE | A | 588 | 11.369 | 5.896 | 8.169 | 1.00 | 24.08 |
| ATOM | 402 | CD2 | PHE | A | 588 | 11.762 | 5.663 | 5.820 | 1.00 | 23.24 |
| ATOM | 403 | CE1 | PHE | A | 588 | 11.526 | 4.515 | 8.336 | 1.00 | 24.41 |
| ATOM | 404 | CE2 | PHE | A | 588 | 11.921 | 4.285 | 5.969 | 1.00 | 23.43 |
| ATOM | 405 | CZ | PHE | A | 588 | 11.804 | 3.707 | 7.228 | 1.00 | 23.76 |
| ATOM | 406 | C | PHE | A | 588 | 12.535 | 10.151 | 6.304 | 1.00 | 30.36 |
| ATOM | 407 | O | PHE | A | 588 | 11.595 | 10.599 | 5.649 | 1.00 | 30.79 |
| ATOM | 408 | N | GLN | A | 589 | 13.462 | 10.912 | 6.873 | 1.00 | 35.42 |
| ATOM | 409 | CA | GLN | A | 589 | 13.489 | 12.366 | 6.750 | 1.00 | 36.58 |
| ATOM | 410 | CB | GLN | A | 589 | 14.167 | 12.739 | 5.433 | 1.00 | 49.72 |
| ATOM | 411 | CG | GLN | A | 589 | 15.501 | 12.043 | 5.243 | 1.00 | 49.96 |
| ATOM | 412 | CD | GLN | A | 589 | 15.680 | 11.505 | 3.839 | 1.00 | 50.27 |
| ATOM | 413 | OE1 | GLN | A | 589 | 14.788 | 10.852 | 3.296 | 1.00 | 50.16 |
| ATOM | 414 | NE2 | GLN | A | 589 | 16.839 | 11.762 | 3.249 | 1.00 | 50.04 |
| ATOM | 415 | C | GLN | A | 589 | 12.133 | 13.055 | 6.846 | 1.00 | 36.29 |
| ATOM | 416 | O | GLN | A | 589 | 11.648 | 13.655 | 5.882 | 1.00 | 36.29 |
| ATOM | 417 | N | MET | A | 590 | 11.524 | 12.969 | 8.021 | 1.00 | 36.14 |
| ATOM | 418 | CA | MET | A | 590 | 10.240 | 13.608 | 8.258 | 1.00 | 35.67 |
| ATOM | 419 | CB | MET | A | 590 | 9.459 | 12.847 | 9.331 | 1.00 | 32.44 |
| ATOM | 420 | CG | MET | A | 590 | 9.055 | 11.431 | 8.966 | 1.00 | 31.88 |
| ATOM | 421 | SD | MET | A | 590 | 8.254 | 10.607 | 10.373 | 1.00 | 31.19 |
| ATOM | 422 | CE | MET | A | 590 | 6.882 | 11.641 | 10.615 | 1.00 | 32.88 |
| ATOM | 423 | C | MET | A | 590 | 10.515 | 15.022 | 8.759 | 1.00 | 35.35 |
| ATOM | 424 | O | MET | A | 590 | 11.351 | 15.209 | 9.641 | 1.00 | 35.80 |
| ATOM | 425 | N | LYS | A | 591 | 9.839 | 16.014 | 8.191 | 1.00 | 35.07 |
| ATOM | 426 | CA | LYS | A | 591 | 10.016 | 17.389 | 8.648 | 1.00 | 34.67 |
| ATOM | 427 | CB | LYS | A | 591 | 9.427 | 18.381 | 7.641 | 1.00 | 46.77 |
| ATOM | 428 | CG | LYS | A | 591 | 10.350 | 18.721 | 6.480 | 1.00 | 47.35 |
| ATOM | 429 | CD | LYS | A | 591 | 10.650 | 17.509 | 5.612 | 1.00 | 47.48 |
| ATOM | 430 | CE | LYS | A | 591 | 11.738 | 17.813 | 4.587 | 1.00 | 47.57 |
| ATOM | 431 | NZ | LYS | A | 591 | 11.398 | 18.958 | 3.694 | 1.00 | 47.52 |
| ATOM | 432 | C | LYS | A | 591 | 9.274 | 17.492 | 9.976 | 1.00 | 33.93 |
| ATOM | 433 | O | LYS | A | 591 | 8.132 | 17.049 | 10.081 | 1.00 | 34.06 |
| ATOM | 434 | N | HIS | A | 592 | 9.926 | 18.068 | 10.983 | 1.00 | 27.75 |
| ATOM | 435 | CA | HIS | A | 592 | 9.332 | 18.205 | 12.310 | 1.00 | 26.86 |
| ATOM | 436 | CB | HIS | A | 592 | 10.227 | 19.071 | 13.208 | 1.00 | 27.82 |
| ATOM | 437 | CG | HIS | A | 592 | 9.770 | 19.139 | 14.632 | 1.00 | 26.72 |
| ATOM | 438 | CD2 | HIS | A | 592 | 9.317 | 20.178 | 15.374 | 1.00 | 27.57 |
| ATOM | 439 | ND1 | HIS | A | 592 | 9.755 | 18.040 | 15.464 | 1.00 | 27.40 |
| ATOM | 440 | CE1 | HIS | A | 592 | 9.314 | 18.399 | 16.657 | 1.00 | 27.96 |
| ATOM | 441 | NE2 | HIS | A | 592 | 9.041 | 19.692 | 16.628 | 1.00 | 27.67 |
| ATOM | 442 | C | HIS | A | 592 | 7.927 | 18.796 | 12.277 | 1.00 | 28.09 |
| ATOM | 443 | O | HIS | A | 592 | 7.021 | 18.288 | 12.933 | 1.00 | 26.68 |
| ATOM | 444 | N | GLU | A | 593 | 7.749 | 19.865 | 11.508 | 1.00 | 32.89 |
| ATOM | 445 | CA | GLU | A | 593 | 6.456 | 20.534 | 11.405 | 1.00 | 34.01 |
| ATOM | 446 | CB | GLU | A | 593 | 6.572 | 21.771 | 10.510 | 1.00 | 53.17 |
| ATOM | 447 | CG | GLU | A | 593 | 7.483 | 22.870 | 11.051 | 1.00 | 53.98 |
| ATOM | 448 | CD | GLU | A | 593 | 8.914 | 22.403 | 11.272 | 1.00 | 54.08 |
| ATOM | 449 | OE1 | GLU | A | 593 | 9.519 | 21.853 | 10.326 | 1.00 | 53.79 |
| ATOM | 450 | OE2 | GLU | A | 593 | 9.434 | 22.591 | 12.394 | 1.00 | 53.97 |
| ATOM | 451 | C | GLU | A | 593 | 5.370 | 19.613 | 10.856 | 1.00 | 33.01 |
| ATOM | 452 | O | GLU | A | 593 | 4.230 | 19.634 | 11.319 | 1.00 | 32.84 |
| ATOM | 453 | N | VAL | A | 594 | 5.730 | 18.808 | 9.864 | 1.00 | 27.04 |
| ATOM | 454 | CA | VAL | A | 594 | 4.783 | 17.886 | 9.249 | 1.00 | 25.85 |
| ATOM | 455 | CB | VAL | A | 594 | 5.382 | 17.279 | 7.962 | 1.00 | 24.72 |
| ATOM | 456 | CG1 | VAL | A | 594 | 4.446 | 16.233 | 7.381 | 1.00 | 24.57 |
| ATOM | 457 | CG2 | VAL | A | 594 | 5.631 | 18.383 | 6.945 | 1.00 | 24.02 |
| ATOM | 458 | C | VAL | A | 594 | 4.386 | 16.771 | 10.215 | 1.00 | 26.36 |
| ATOM | 459 | O | VAL | A | 594 | 3.214 | 16.386 | 10.283 | 1.00 | 25.05 |
| ATOM | 460 | N | LEU | A | 595 | 5.356 | 16.246 | 10.956 | 1.00 | 24.15 |
| ATOM | 461 | CA | LEU | A | 595 | 5.062 | 15.187 | 11.918 | 1.00 | 25.41 |
| ATOM | 462 | CB | LEU | A | 595 | 6.352 | 14.633 | 12.536 | 1.00 | 24.61 |
| ATOM | 463 | CG | LEU | A | 595 | 6.204 | 13.640 | 13.696 | 1.00 | 25.86 |
| ATOM | 464 | CD1 | LEU | A | 595 | 5.345 | 12.453 | 13.272 | 1.00 | 24.75 |
| ATOM | 465 | CD2 | LEU | A | 595 | 7.577 | 13.163 | 14.141 | 1.00 | 25.05 |
| ATOM | 466 | C | LEU | A | 595 | 4.148 | 15.745 | 13.009 | 1.00 | 25.02 |
| ATOM | 467 | O | LEU | A | 595 | 3.197 | 15.088 | 13.435 | 1.00 | 24.20 |
| ATOM | 468 | N | CYS | A | 596 | 4.426 | 16.970 | 13.451 | 1.00 | 24.36 |
| ATOM | 469 | CA | CYS | A | 596 | 3.603 | 17.588 | 14.479 | 1.00 | 24.03 |
| ATOM | 470 | CB | CYS | A | 596 | 4.195 | 18.931 | 14.907 | 1.00 | 27.15 |
| ATOM | 471 | SG | CYS | A | 596 | 5.699 | 18.771 | 15.892 | 1.00 | 26.30 |
| ATOM | 472 | C | CYS | A | 596 | 2.177 | 17.789 | 13.987 | 1.00 | 24.31 |
| ATOM | 473 | O | CYS | A | 596 | 1.222 | 17.537 | 14.721 | 1.00 | 24.29 |
| ATOM | 474 | N | ARG | A | 597 | 2.032 | 18.233 | 12.742 | 1.00 | 22.43 |
| ATOM | 475 | CA | ARG | A | 597 | 0.703 | 18.460 | 12.194 | 1.00 | 22.27 |
| ATOM | 476 | CB | ARG | A | 597 | 0.777 | 19.177 | 10.841 | 1.00 | 37.36 |
| ATOM | 477 | CG | ARG | A | 597 | −0.597 | 19.576 | 10.315 | 1.00 | 38.44 |
| ATOM | 478 | CD | ARG | A | 597 | −0.541 | 20.392 | 9.032 | 1.00 | 38.82 |
| ATOM | 479 | NE | ARG | A | 597 | 0.033 | 21.719 | 9.233 | 1.00 | 38.78 |
| ATOM | 480 | CZ | ARG | A | 597 | 1.286 | 22.043 | 8.936 | 1.00 | 38.71 |
| ATOM | 481 | NH1 | ARG | A | 597 | 2.101 | 21.133 | 8.421 | 1.00 | 38.58 |
| ATOM | 482 | NH2 | ARG | A | 597 | 1.721 | 23.278 | 9.149 | 1.00 | 38.90 |
| ATOM | 483 | C | ARG | A | 597 | −0.042 | 17.140 | 12.042 | 1.00 | 21.87 |
| ATOM | 484 | O | ARG | A | 597 | −1.231 | 17.049 | 12.347 | 1.00 | 22.25 |
| ATOM | 485 | N | TRP | A | 598 | 0.661 | 16.114 | 11.578 | 1.00 | 22.62 |
| ATOM | 486 | CA | TRP | A | 598 | 0.039 | 14.802 | 11.396 | 1.00 | 20.68 |
| ATOM | 487 | CB | TRP | A | 598 | 1.048 | 13.826 | 10.788 | 1.00 | 21.19 |
| ATOM | 488 | CG | TRP | A | 598 | 0.506 | 12.443 | 10.641 | 1.00 | 20.61 |
| ATOM | 489 | CD2 | TRP | A | 598 | 0.680 | 11.358 | 11.554 | 1.00 | 21.45 |
| ATOM | 490 | CE2 | TRP | A | 598 | −0.059 | 10.262 | 11.048 | 1.00 | 20.54 |
| ATOM | 491 | CE3 | TRP | A | 598 | 1.386 | 11.201 | 12.755 | 1.00 | 20.69 |
| ATOM | 492 | CD1 | TRP | A | 598 | −0.304 | 11.982 | 9.644 | 1.00 | 21.12 |
| ATOM | 493 | NE1 | TRP | A | 598 | −0.648 | 10.671 | 9.882 | 1.00 | 21.75 |
| ATOM | 494 | CZ2 | TRP | A | 598 | −0.111 | 9.028 | 11.701 | 1.00 | 20.03 |
| ATOM | 495 | CZ3 | TRP | A | 598 | 1.334 | 9.974 | 13.406 | 1.00 | 21.23 |
| ATOM | 496 | CH2 | TRP | A | 598 | 0.588 | 8.901 | 12.875 | 1.00 | 21.56 |
| ATOM | 497 | C | TRP | A | 598 | −0.461 | 14.256 | 12.734 | 1.00 | 20.83 |
| ATOM | 498 | O | TRP | A | 598 | −1.575 | 13.743 | 12.833 | 1.00 | 20.85 |
| ATOM | 499 | N | ILE | A | 599 | 0.371 | 14.362 | 13.762 | 1.00 | 18.38 |
| ATOM | 500 | CA | ILE | A | 599 | −0.001 | 13.881 | 15.081 | 1.00 | 17.01 |
| ATOM | 501 | CB | ILE | A | 599 | 1.138 | 14.106 | 16.103 | 1.00 | 18.07 |
| ATOM | 502 | CG2 | ILE | A | 599 | 0.625 | 13.881 | 17.516 | 1.00 | 18.87 |
| ATOM | 503 | CG1 | ILE | A | 599 | 2.304 | 13.158 | 15.794 | 1.00 | 18.94 |
| ATOM | 504 | CD1 | ILE | A | 599 | 3.523 | 13.367 | 16.669 | 1.00 | 18.33 |
| ATOM | 505 | C | ILE | A | 599 | −1.263 | 14.596 | 15.554 | 1.00 | 18.40 |
| ATOM | 506 | O | ILE | A | 599 | −2.184 | 13.969 | 16.081 | 1.00 | 16.74 |
| ATOM | 507 | N | LEU | A | 600 | −1.311 | 15.912 | 15.371 | 1.00 | 21.06 |
| ATOM | 508 | CA | LEU | A | 600 | −2.486 | 16.657 | 15.802 | 1.00 | 21.13 |
| ATOM | 509 | CB | LEU | A | 600 | −2.202 | 18.162 | 15.761 | 1.00 | 21.76 |
| ATOM | 510 | CG | LEU | A | 600 | −1.149 | 18.582 | 16.788 | 1.00 | 21.76 |
| ATOM | 511 | CD1 | LEU | A | 600 | −0.811 | 20.053 | 16.597 | 1.00 | 21.85 |
| ATOM | 512 | CD2 | LEU | A | 600 | −1.672 | 18.324 | 18.201 | 1.00 | 22.51 |
| ATOM | 513 | C | LEU | A | 600 | −3.728 | 16.325 | 14.979 | 1.00 | 20.73 |
| ATOM | 514 | O | LEU | A | 600 | −4.841 | 16.351 | 15.501 | 1.00 | 21.49 |
| ATOM | 515 | N | SER | A | 601 | −3.540 | 16.032 | 13.694 | 1.00 | 21.08 |
| ATOM | 516 | CA | SER | A | 601 | −4.665 | 15.674 | 12.833 | 1.00 | 20.75 |
| ATOM | 517 | CB | SER | A | 601 | −4.225 | 15.579 | 11.372 | 1.00 | 22.55 |
| ATOM | 518 | OG | SER | A | 601 | −4.008 | 16.869 | 10.822 | 1.00 | 23.14 |
| ATOM | 519 | C | SER | A | 601 | −5.228 | 14.333 | 13.287 | 1.00 | 20.19 |
| ATOM | 520 | O | SER | A | 601 | −6.438 | 14.125 | 13.275 | 1.00 | 20.13 |
| ATOM | 521 | N | VAL | A | 602 | −4.340 | 13.424 | 13.679 | 1.00 | 21.43 |
| ATOM | 522 | CA | VAL | A | 602 | −4.773 | 12.116 | 14.168 | 1.00 | 21.85 |
| ATOM | 523 | CB | VAL | A | 602 | −3.564 | 11.204 | 14.471 | 1.00 | 17.69 |
| ATOM | 524 | CG1 | VAL | A | 602 | −4.018 | 9.928 | 15.198 | 1.00 | 18.88 |
| ATOM | 525 | CG2 | VAL | A | 602 | −2.867 | 10.845 | 13.174 | 1.00 | 19.25 |
| ATOM | 526 | C | VAL | A | 602 | −5.581 | 12.312 | 15.445 | 1.00 | 21.58 |
| ATOM | 527 | O | VAL | A | 602 | −6.705 | 11.823 | 15.566 | 1.00 | 20.63 |
| ATOM | 528 | N | LYS | A | 603 | −5.011 | 13.052 | 16.394 | 1.00 | 20.30 |
| ATOM | 529 | CA | LYS | A | 603 | −5.697 | 13.303 | 17.655 | 1.00 | 19.93 |
| ATOM | 530 | CB | LYS | A | 603 | −4.833 | 14.186 | 18.567 | 1.00 | 29.59 |
| ATOM | 531 | CG | LYS | A | 603 | −5.553 | 14.663 | 19.829 | 1.00 | 29.47 |
| ATOM | 532 | CD | LYS | A | 603 | −4.587 | 15.206 | 20.882 | 1.00 | 30.01 |
| ATOM | 533 | CE | LYS | A | 603 | −3.846 | 16.449 | 20.416 | 1.00 | 30.55 |
| ATOM | 534 | NZ | LYS | A | 603 | −2.899 | 16.934 | 21.464 | 1.00 | 31.23 |
| ATOM | 535 | C | LYS | A | 603 | −7.052 | 13.968 | 17.430 | 1.00 | 19.43 |
| ATOM | 536 | O | LYS | A | 603 | −8.034 | 13.636 | 18.095 | 1.00 | 20.76 |
| ATOM | 537 | N | LYS | A | 604 | −7.100 | 14.894 | 16.481 | 1.00 | 19.98 |
| ATOM | 538 | CA | LYS | A | 604 | −8.325 | 15.629 | 16.174 | 1.00 | 21.57 |
| ATOM | 539 | CB | LYS | A | 604 | −8.024 | 16.775 | 15.207 | 1.00 | 38.76 |
| ATOM | 540 | CG | LYS | A | 604 | −9.262 | 17.531 | 14.741 | 1.00 | 39.38 |
| ATOM | 541 | CD | LYS | A | 604 | −8.910 | 18.610 | 13.732 | 1.00 | 39.39 |
| ATOM | 542 | CE | LYS | A | 604 | −10.158 | 19.340 | 13.255 | 1.00 | 39.74 |
| ATOM | 543 | NZ | LYS | A | 604 | −9.843 | 20.385 | 12.242 | 1.00 | 39.72 |
| ATOM | 544 | C | LYS | A | 604 | −9.413 | 14.754 | 15.579 | 1.00 | 20.90 |
| ATOM | 545 | O | LYS | A | 604 | −10.609 | 15.039 | 15.742 | 1.00 | 21.11 |
| ATOM | 546 | N | ASN | A | 605 | −9.009 | 13.688 | 14.892 | 1.00 | 21.37 |
| ATOM | 547 | CA | ASN | A | 605 | −9.987 | 12.812 | 14.273 | 1.00 | 21.04 |
| ATOM | 548 | CB | ASN | A | 605 | −9.486 | 12.315 | 12.920 | 1.00 | 27.07 |
| ATOM | 549 | CG | ASN | A | 605 | −9.635 | 13.366 | 11.836 | 1.00 | 27.98 |
| ATOM | 550 | OD1 | ASN | A | 605 | −8.775 | 14.234 | 11.666 | 1.00 | 27.85 |
| ATOM | 551 | ND2 | ASN | A | 605 | −10.745 | 13.308 | 11.112 | 1.00 | 27.52 |
| ATOM | 552 | C | ASN | A | 605 | −10.506 | 11.662 | 15.116 | 1.00 | 21.57 |
| ATOM | 553 | O | ASN | A | 605 | −11.072 | 10.702 | 14.590 | 1.00 | 23.07 |
| ATOM | 554 | N | TYR | A | 606 | −10.292 | 11.754 | 16.422 | 1.00 | 20.07 |
| ATOM | 555 | CA | TYR | A | 606 | −10.853 | 10.792 | 17.349 | 1.00 | 20.46 |
| ATOM | 556 | CB | TYR | A | 606 | −9.858 | 10.374 | 18.437 | 1.00 | 18.79 |
| ATOM | 557 | CG | TYR | A | 606 | −8.993 | 9.216 | 17.992 | 1.00 | 18.19 |
| ATOM | 558 | CD1 | TYR | A | 606 | −7.745 | 9.430 | 17.409 | 1.00 | 19.08 |
| ATOM | 559 | CE1 | TYR | A | 606 | −6.989 | 8.362 | 16.913 | 1.00 | 18.94 |
| ATOM | 560 | CD2 | TYR | A | 606 | −9.467 | 7.909 | 18.074 | 1.00 | 18.23 |
| ATOM | 561 | CE2 | TYR | A | 606 | −8.722 | 6.840 | 17.578 | 1.00 | 17.63 |
| ATOM | 562 | CZ | TYR | A | 606 | −7.485 | 7.077 | 16.999 | 1.00 | 18.23 |
| ATOM | 563 | OH | TYR | A | 606 | −6.747 | 6.029 | 16.489 | 1.00 | 15.86 |
| ATOM | 564 | C | TYR | A | 606 | −12.015 | 11.580 | 17.947 | 1.00 | 21.76 |
| ATOM | 565 | O | TYR | A | 606 | −11.975 | 12.819 | 18.007 | 1.00 | 20.70 |
| ATOM | 566 | N | ARG | A | 607 | −13.060 | 10.882 | 18.365 | 1.00 | 19.57 |
| ATOM | 567 | CA | ARG | A | 607 | −14.223 | 11.562 | 18.931 | 1.00 | 20.12 |
| ATOM | 568 | CB | ARG | A | 607 | −15.497 | 10.791 | 18.575 | 1.00 | 23.54 |
| ATOM | 569 | CG | ARG | A | 607 | −15.679 | 10.605 | 17.074 | 1.00 | 23.38 |
| ATOM | 570 | CD | ARG | A | 607 | −17.034 | 10.010 | 16.715 | 1.00 | 23.37 |
| ATOM | 571 | NE | ARG | A | 607 | −17.142 | 9.734 | 15.282 | 1.00 | 24.11 |
| ATOM | 572 | CZ | ARG | A | 607 | −18.274 | 9.408 | 14.665 | 1.00 | 24.11 |
| ATOM | 573 | NH1 | ARG | A | 607 | −19.405 | 9.321 | 15.355 | 1.00 | 24.42 |
| ATOM | 574 | NH2 | ARG | A | 607 | −18.274 | 9.163 | 13.362 | 1.00 | 25.10 |
| ATOM | 575 | C | ARG | A | 607 | −14.037 | 11.647 | 20.438 | 1.00 | 20.33 |
| ATOM | 576 | O | ARG | A | 607 | −14.168 | 10.648 | 21.150 | 1.00 | 20.09 |
| ATOM | 577 | N | LYS | A | 608 | −13.736 | 12.847 | 20.926 | 1.00 | 25.24 |
| ATOM | 578 | CA | LYS | A | 608 | −13.484 | 13.033 | 22.349 | 1.00 | 25.95 |
| ATOM | 579 | CB | LYS | A | 608 | −13.108 | 14.491 | 22.636 | 1.00 | 41.13 |
| ATOM | 580 | CG | LYS | A | 608 | −14.175 | 15.511 | 22.294 | 1.00 | 41.73 |
| ATOM | 581 | CD | LYS | A | 608 | −13.670 | 16.916 | 22.603 | 1.00 | 41.87 |
| ATOM | 582 | CE | LYS | A | 608 | −14.750 | 17.972 | 22.413 | 1.00 | 41.79 |
| ATOM | 583 | NZ | LYS | A | 608 | −14.231 | 19.340 | 22.703 | 1.00 | 41.73 |
| ATOM | 584 | C | LYS | A | 608 | −14.625 | 12.599 | 23.254 | 1.00 | 26.44 |
| ATOM | 585 | O | LYS | A | 608 | −14.397 | 12.225 | 24.405 | 1.00 | 25.96 |
| ATOM | 586 | N | ASN | A | 609 | −15.847 | 12.626 | 22.732 | 1.00 | 27.23 |
| ATOM | 587 | CA | ASN | A | 609 | −17.005 | 12.248 | 23.522 | 1.00 | 27.32 |
| ATOM | 588 | CB | ASN | A | 609 | −18.212 | 13.066 | 23.071 | 1.00 | 36.30 |
| ATOM | 589 | CG | ASN | A | 609 | −17.995 | 14.554 | 23.282 | 1.00 | 36.84 |
| ATOM | 590 | OD1 | ASN | A | 609 | −17.809 | 15.008 | 24.411 | 1.00 | 37.02 |
| ATOM | 591 | ND2 | ASN | A | 609 | −17.998 | 15.316 | 22.199 | 1.00 | 37.30 |
| ATOM | 592 | C | ASN | A | 609 | −17.317 | 10.758 | 23.541 | 1.00 | 26.80 |
| ATOM | 593 | O | ASN | A | 609 | −18.330 | 10.332 | 24.098 | 1.00 | 27.72 |
| ATOM | 594 | N | VAL | A | 610 | −16.451 | 9.964 | 22.920 | 1.00 | 21.65 |
| ATOM | 595 | CA | VAL | A | 610 | −16.616 | 8.512 | 22.946 | 1.00 | 20.92 |
| ATOM | 596 | CB | VAL | A | 610 | −16.097 | 7.862 | 21.645 | 1.00 | 20.35 |
| ATOM | 597 | CG1 | VAL | A | 610 | −15.897 | 6.370 | 21.846 | 1.00 | 20.76 |
| ATOM | 598 | CG2 | VAL | A | 610 | −17.120 | 8.086 | 20.522 | 1.00 | 18.26 |
| ATOM | 599 | C | VAL | A | 610 | −15.780 | 8.109 | 24.171 | 1.00 | 19.89 |
| ATOM | 600 | O | VAL | A | 610 | −14.590 | 8.414 | 24.254 | 1.00 | 20.96 |
| ATOM | 601 | N | ALA | A | 611 | −16.423 | 7.450 | 25.130 | 1.00 | 19.80 |
| ATOM | 602 | CA | ALA | A | 611 | −15.791 | 7.078 | 26.397 | 1.00 | 19.90 |
| ATOM | 603 | CB | ALA | A | 611 | −16.780 | 6.287 | 27.251 | 1.00 | 20.34 |
| ATOM | 604 | C | ALA | A | 611 | −14.448 | 6.363 | 26.383 | 1.00 | 19.34 |
| ATOM | 605 | O | ALA | A | 611 | −13.549 | 6.731 | 27.138 | 1.00 | 20.80 |
| ATOM | 606 | N | TYR | A | 612 | −14.307 | 5.343 | 25.542 | 1.00 | 18.10 |
| ATOM | 607 | CA | TYR | A | 612 | −13.060 | 4.599 | 25.512 | 1.00 | 15.69 |
| ATOM | 608 | CB | TYR | A | 612 | −13.321 | 3.134 | 25.873 | 1.00 | 20.49 |
| ATOM | 609 | CG | TYR | A | 612 | −12.107 | 2.251 | 25.709 | 1.00 | 20.53 |
| ATOM | 610 | CD1 | TYR | A | 612 | −12.047 | 1.304 | 24.685 | 1.00 | 21.50 |
| ATOM | 611 | CE1 | TYR | A | 612 | −10.932 | 0.484 | 24.536 | 1.00 | 20.27 |
| ATOM | 612 | CD2 | TYR | A | 612 | −11.020 | 2.359 | 26.577 | 1.00 | 20.70 |
| ATOM | 613 | CE2 | TYR | A | 612 | −9.906 | 1.546 | 26.434 | 1.00 | 19.44 |
| ATOM | 614 | CZ | TYR | A | 612 | −9.870 | 0.611 | 25.414 | 1.00 | 20.76 |
| ATOM | 615 | OH | TYR | A | 612 | −8.769 | −0.205 | 25.284 | 1.00 | 20.43 |
| ATOM | 616 | C | TYR | A | 612 | −12.264 | 4.682 | 24.218 | 1.00 | 14.59 |
| ATOM | 617 | O | TYR | A | 612 | −11.064 | 4.921 | 24.268 | 1.00 | 15.21 |
| ATOM | 618 | N | HIS | A | 613 | −12.915 | 4.500 | 23.069 | 1.00 | 16.35 |
| ATOM | 619 | CA | HIS | A | 613 | −12.197 | 4.553 | 21.793 | 1.00 | 15.66 |
| ATOM | 620 | CB | HIS | A | 613 | −12.942 | 3.763 | 20.705 | 1.00 | 19.05 |
| ATOM | 621 | CG | HIS | A | 613 | −12.971 | 2.286 | 20.941 | 1.00 | 21.28 |
| ATOM | 622 | CD2 | HIS | A | 613 | −12.010 | 1.341 | 20.797 | 1.00 | 20.01 |
| ATOM | 623 | ND1 | HIS | A | 613 | −14.087 | 1.628 | 21.408 | 1.00 | 19.61 |
| ATOM | 624 | CE1 | HIS | A | 613 | −13.816 | 0.342 | 21.541 | 1.00 | 20.40 |
| ATOM | 625 | NE2 | HIS | A | 613 | −12.562 | 0.140 | 21.176 | 1.00 | 20.73 |
| ATOM | 626 | C | HIS | A | 613 | −11.998 | 5.989 | 21.330 | 1.00 | 15.17 |
| ATOM | 627 | O | HIS | A | 613 | −12.633 | 6.451 | 20.376 | 1.00 | 17.00 |
| ATOM | 628 | N | ASN | A | 614 | −11.101 | 6.682 | 22.020 | 1.00 | 18.64 |
| ATOM | 629 | CA | ASN | A | 614 | −10.784 | 8.068 | 21.725 | 1.00 | 17.28 |
| ATOM | 630 | CB | ASN | A | 614 | −11.344 | 8.977 | 22.827 | 1.00 | 21.97 |
| ATOM | 631 | CG | ASN | A | 614 | −10.939 | 8.530 | 24.219 | 1.00 | 21.21 |
| ATOM | 632 | OD1 | ASN | A | 614 | −9.752 | 8.417 | 24.526 | 1.00 | 22.66 |
| ATOM | 633 | ND2 | ASN | A | 614 | −11.928 | 8.277 | 25.074 | 1.00 | 21.32 |
| ATOM | 634 | C | ASN | A | 614 | −9.265 | 8.207 | 21.628 | 1.00 | 18.30 |
| ATOM | 635 | O | ASN | A | 614 | −8.543 | 7.210 | 21.671 | 1.00 | 17.11 |
| ATOM | 636 | N | TRP | A | 615 | −8.773 | 9.434 | 21.504 | 1.00 | 20.23 |
| ATOM | 637 | CA | TRP | A | 615 | −7.335 | 9.650 | 21.388 | 1.00 | 20.84 |
| ATOM | 638 | CB | TRP | A | 615 | −7.020 | 11.152 | 21.346 | 1.00 | 18.84 |
| ATOM | 639 | CG | TRP | A | 615 | −5.567 | 11.469 | 21.610 | 1.00 | 20.81 |
| ATOM | 640 | CD2 | TRP | A | 615 | −4.446 | 11.146 | 20.775 | 1.00 | 20.87 |
| ATOM | 641 | CE2 | TRP | A | 615 | −3.287 | 11.632 | 21.427 | 1.00 | 20.24 |
| ATOM | 642 | CE3 | TRP | A | 615 | −4.307 | 10.493 | 19.542 | 1.00 | 20.69 |
| ATOM | 643 | CD1 | TRP | A | 615 | −5.055 | 12.115 | 22.700 | 1.00 | 19.88 |
| ATOM | 644 | NE1 | TRP | A | 615 | −3.689 | 12.217 | 22.599 | 1.00 | 19.37 |
| ATOM | 645 | CZ2 | TRP | A | 615 | −2.007 | 11.488 | 20.886 | 1.00 | 21.34 |
| ATOM | 646 | CZ3 | TRP | A | 615 | −3.031 | 10.346 | 19.004 | 1.00 | 21.28 |
| ATOM | 647 | CH2 | TRP | A | 615 | −1.897 | 10.845 | 19.679 | 1.00 | 21.14 |
| ATOM | 648 | C | TRP | A | 615 | −6.457 | 8.993 | 22.453 | 1.00 | 19.80 |
| ATOM | 649 | O | TRP | A | 615 | −5.383 | 8.479 | 22.133 | 1.00 | 19.45 |
| ATOM | 650 | N | ARG | A | 616 | −6.888 | 9.010 | 23.713 | 1.00 | 19.15 |
| ATOM | 651 | CA | ARG | A | 616 | −6.063 | 8.424 | 24.764 | 1.00 | 18.73 |
| ATOM | 652 | CB | ARG | A | 616 | −6.658 | 8.707 | 26.146 | 1.00 | 20.84 |
| ATOM | 653 | CG | ARG | A | 616 | −6.628 | 10.197 | 26.516 | 1.00 | 20.98 |
| ATOM | 654 | CD | ARG | A | 616 | −5.208 | 10.754 | 26.511 | 1.00 | 21.78 |
| ATOM | 655 | NE | ARG | A | 616 | −5.185 | 12.195 | 26.772 | 1.00 | 22.05 |
| ATOM | 656 | CZ | ARG | A | 616 | −4.519 | 12.760 | 27.773 | 1.00 | 22.62 |
| ATOM | 657 | NH1 | ARG | A | 616 | −3.820 | 12.012 | 28.610 | 1.00 | 22.11 |
| ATOM | 658 | NH2 | ARG | A | 616 | −4.558 | 14.074 | 27.936 | 1.00 | 22.34 |
| ATOM | 659 | C | ARG | A | 616 | −5.869 | 6.922 | 24.559 | 1.00 | 18.23 |
| ATOM | 660 | O | ARG | A | 616 | −4.799 | 6.388 | 24.857 | 1.00 | 18.43 |
| ATOM | 661 | N | HIS | A | 617 | −6.889 | 6.246 | 24.041 | 1.00 | 17.23 |
| ATOM | 662 | CA | HIS | A | 617 | −6.754 | 4.812 | 23.796 | 1.00 | 17.41 |
| ATOM | 663 | CB | HIS | A | 617 | −8.095 | 4.194 | 23.403 | 1.00 | 16.86 |
| ATOM | 664 | CG | HIS | A | 617 | −7.964 | 2.830 | 22.798 | 1.00 | 13.76 |
| ATOM | 665 | CD2 | HIS | A | 617 | −8.291 | 2.369 | 21.569 | 1.00 | 17.85 |
| ATOM | 666 | ND1 | HIS | A | 617 | −7.384 | 1.772 | 23.465 | 1.00 | 17.12 |
| ATOM | 667 | CE1 | HIS | A | 617 | −7.357 | 0.716 | 22.670 | 1.00 | 15.55 |
| ATOM | 668 | NE2 | HIS | A | 617 | −7.899 | 1.052 | 21.515 | 1.00 | 16.82 |
| ATOM | 669 | C | HIS | A | 617 | −5.737 | 4.566 | 22.682 | 1.00 | 17.79 |
| ATOM | 670 | O | HIS | A | 617 | −4.911 | 3.643 | 22.770 | 1.00 | 17.66 |
| ATOM | 671 | N | ALA | A | 618 | −5.807 | 5.384 | 21.635 | 1.00 | 16.69 |
| ATOM | 672 | CA | ALA | A | 618 | −4.897 | 5.268 | 20.499 | 1.00 | 17.60 |
| ATOM | 673 | CB | ALA | A | 618 | −5.268 | 6.270 | 19.416 | 1.00 | 19.06 |
| ATOM | 674 | C | ALA | A | 618 | −3.478 | 5.517 | 20.973 | 1.00 | 18.60 |
| ATOM | 675 | O | ALA | A | 618 | −2.553 | 4.777 | 20.642 | 1.00 | 16.65 |
| ATOM | 676 | N | PHE | A | 619 | −3.327 | 6.573 | 21.764 | 1.00 | 16.36 |
| ATOM | 677 | CA | PHE | A | 619 | −2.040 | 6.942 | 22.316 | 1.00 | 18.60 |
| ATOM | 678 | CB | PHE | A | 619 | −2.205 | 8.224 | 23.142 | 1.00 | 21.01 |
| ATOM | 679 | CG | PHE | A | 619 | −0.989 | 8.594 | 23.938 | 1.00 | 22.31 |
| ATOM | 680 | CD1 | PHE | A | 619 | 0.166 | 9.041 | 23.305 | 1.00 | 21.99 |
| ATOM | 681 | CD2 | PHE | A | 619 | −0.995 | 8.475 | 25.326 | 1.00 | 22.04 |
| ATOM | 682 | CE1 | PHE | A | 619 | 1.304 | 9.366 | 24.045 | 1.00 | 21.42 |
| ATOM | 683 | CE2 | PHE | A | 619 | 0.139 | 8.798 | 26.076 | 1.00 | 21.94 |
| ATOM | 684 | CZ | PHE | A | 619 | 1.292 | 9.246 | 25.426 | 1.00 | 21.32 |
| ATOM | 685 | C | PHE | A | 619 | −1.459 | 5.816 | 23.176 | 1.00 | 17.86 |
| ATOM | 686 | O | PHE | A | 619 | −0.285 | 5.470 | 23.040 | 1.00 | 17.74 |
| ATOM | 687 | N | ASN | A | 620 | −2.281 | 5.239 | 24.049 | 1.00 | 17.68 |
| ATOM | 688 | CA | ASN | A | 620 | −1.814 | 4.160 | 24.920 | 1.00 | 18.84 |
| ATOM | 689 | CB | ASN | A | 620 | −2.868 | 3.793 | 25.972 | 1.00 | 22.20 |
| ATOM | 690 | CG | ASN | A | 620 | −3.028 | 4.867 | 27.037 | 1.00 | 22.41 |
| ATOM | 691 | OD1 | ASN | A | 620 | −2.078 | 5.578 | 27.354 | 1.00 | 22.93 |
| ATOM | 692 | ND2 | ASN | A | 620 | −4.220 | 4.969 | 27.611 | 1.00 | 21.57 |
| ATOM | 693 | C | ASN | A | 620 | −1.450 | 2.921 | 24.113 | 1.00 | 18.91 |
| ATOM | 694 | O | ASN | A | 620 | −0.514 | 2.201 | 24.461 | 1.00 | 18.42 |
| ATOM | 695 | N | THR | A | 621 | −2.184 | 2.682 | 23.031 | 1.00 | 16.41 |
| ATOM | 696 | CA | THR | A | 621 | −1.901 | 1.519 | 22.191 | 1.00 | 16.82 |
| ATOM | 697 | CB | THR | A | 621 | −2.965 | 1.360 | 21.093 | 1.00 | 17.95 |
| ATOM | 698 | OG1 | THR | A | 621 | −4.246 | 1.190 | 21.704 | 1.00 | 17.42 |
| ATOM | 699 | CG2 | THR | A | 621 | −2.664 | 0.130 | 20.233 | 1.00 | 17.02 |
| ATOM | 700 | C | THR | A | 621 | −0.526 | 1.693 | 21.556 | 1.00 | 18.34 |
| ATOM | 701 | O | THR | A | 621 | 0.276 | 0.749 | 21.505 | 1.00 | 18.19 |
| ATOM | 702 | N | ALA | A | 622 | −0.249 | 2.901 | 21.085 | 1.00 | 15.98 |
| ATOM | 703 | CA | ALA | A | 622 | 1.041 | 3.211 | 20.479 | 1.00 | 18.35 |
| ATOM | 704 | CB | ALA | A | 622 | 1.005 | 4.592 | 19.864 | 1.00 | 16.90 |
| ATOM | 705 | C | ALA | A | 622 | 2.151 | 3.138 | 21.529 | 1.00 | 17.61 |
| ATOM | 706 | O | ALA | A | 622 | 3.242 | 2.656 | 21.248 | 1.00 | 16.57 |
| ATOM | 707 | N | GLN | A | 623 | 1.876 | 3.622 | 22.740 | 1.00 | 17.78 |
| ATOM | 708 | CA | GLN | A | 623 | 2.878 | 3.578 | 23.803 | 1.00 | 19.17 |
| ATOM | 709 | CB | GLN | A | 623 | 2.351 | 4.260 | 25.071 | 1.00 | 19.99 |
| ATOM | 710 | CG | GLN | A | 623 | 3.361 | 4.335 | 26.219 | 1.00 | 19.42 |
| ATOM | 711 | CD | GLN | A | 623 | 3.358 | 3.103 | 27.110 | 1.00 | 20.35 |
| ATOM | 712 | OE1 | GLN | A | 623 | 2.302 | 2.595 | 27.488 | 1.00 | 19.39 |
| ATOM | 713 | NE2 | GLN | A | 623 | 4.547 | 2.634 | 27.475 | 1.00 | 19.48 |
| ATOM | 714 | C | GLN | A | 623 | 3.251 | 2.137 | 24.114 | 1.00 | 19.45 |
| ATOM | 715 | O | GLN | A | 623 | 4.420 | 1.824 | 24.358 | 1.00 | 19.89 |
| ATOM | 716 | N | CYS | A | 624 | 2.258 | 1.256 | 24.118 | 1.00 | 17.26 |
| ATOM | 717 | CA | CYS | A | 624 | 2.533 | −0.146 | 24.385 | 1.00 | 19.37 |
| ATOM | 718 | CB | CYS | A | 624 | 1.234 | −0.938 | 24.530 | 1.00 | 19.88 |
| ATOM | 719 | SG | CYS | A | 624 | 1.499 | −2.658 | 25.026 | 1.00 | 19.62 |
| ATOM | 720 | C | CYS | A | 624 | 3.369 | −0.708 | 23.237 | 1.00 | 18.34 |
| ATOM | 721 | O | CYS | A | 624 | 4.255 | −1.528 | 23.460 | 1.00 | 18.71 |
| ATOM | 722 | N | MET | A | 625 | 3.094 | −0.258 | 22.015 | 1.00 | 19.30 |
| ATOM | 723 | CA | MET | A | 625 | 3.844 | −0.720 | 20.843 | 1.00 | 18.88 |
| ATOM | 724 | CB | MET | A | 625 | 3.259 | −0.102 | 19.567 | 1.00 | 21.69 |
| ATOM | 725 | CG | MET | A | 625 | 3.952 | −0.521 | 18.267 | 1.00 | 19.33 |
| ATOM | 726 | SD | MET | A | 625 | 3.854 | −2.291 | 17.926 | 1.00 | 18.96 |
| ATOM | 727 | CE | MET | A | 625 | 2.148 | −2.414 | 17.292 | 1.00 | 20.28 |
| ATOM | 728 | C | MET | A | 625 | 5.307 | −0.296 | 21.019 | 1.00 | 20.73 |
| ATOM | 729 | O | MET | A | 625 | 6.226 | −1.097 | 20.832 | 1.00 | 19.61 |
| ATOM | 730 | N | PHE | A | 626 | 5.515 | 0.968 | 21.381 | 1.00 | 21.46 |
| ATOM | 731 | CA | PHE | A | 626 | 6.862 | 1.487 | 21.604 | 1.00 | 22.24 |
| ATOM | 732 | CB | PHE | A | 626 | 6.810 | 2.973 | 22.003 | 1.00 | 18.30 |
| ATOM | 733 | CG | PHE | A | 626 | 8.162 | 3.566 | 22.325 | 1.00 | 18.77 |
| ATOM | 734 | CD1 | PHE | A | 626 | 8.662 | 3.539 | 23.628 | 1.00 | 19.77 |
| ATOM | 735 | CD2 | PHE | A | 626 | 8.943 | 4.126 | 21.321 | 1.00 | 19.03 |
| ATOM | 736 | CE1 | PHE | A | 626 | 9.920 | 4.060 | 23.921 | 1.00 | 19.58 |
| ATOM | 737 | CE2 | PHE | A | 626 | 10.207 | 4.650 | 21.607 | 1.00 | 19.50 |
| ATOM | 738 | CZ | PHE | A | 626 | 10.693 | 4.616 | 22.907 | 1.00 | 17.99 |
| ATOM | 739 | C | PHE | A | 626 | 7.578 | 0.684 | 22.685 | 1.00 | 22.81 |
| ATOM | 740 | O | PHE | A | 626 | 8.747 | 0.321 | 22.528 | 1.00 | 22.67 |
| ATOM | 741 | N | ALA | A | 627 | 6.878 | 0.405 | 23.781 | 1.00 | 23.57 |
| ATOM | 742 | CA | ALA | A | 627 | 7.460 | −0.351 | 24.883 | 1.00 | 24.17 |
| ATOM | 743 | CB | ALA | A | 627 | 6.511 | −0.357 | 26.082 | 1.00 | 26.38 |
| ATOM | 744 | C | ALA | A | 627 | 7.794 | −1.787 | 24.477 | 1.00 | 24.26 |
| ATOM | 745 | O | ALA | A | 627 | 8.854 | −2.305 | 24.834 | 1.00 | 25.36 |
| ATOM | 746 | N | ALA | A | 628 | 6.898 | −2.428 | 23.728 | 1.00 | 21.99 |
| ATOM | 747 | CA | ALA | A | 628 | 7.127 | −3.806 | 23.296 | 1.00 | 22.22 |
| ATOM | 748 | CB | ALA | A | 628 | 5.884 | −4.364 | 22.609 | 1.00 | 18.57 |
| ATOM | 749 | C | ALA | A | 628 | 8.315 | −3.879 | 22.350 | 1.00 | 23.09 |
| ATOM | 750 | O | ALA | A | 628 | 9.085 | −4.838 | 22.382 | 1.00 | 22.87 |
| ATOM | 751 | N | LEU | A | 629 | 8.462 | −2.864 | 21.506 | 1.00 | 21.79 |
| ATOM | 752 | CA | LEU | A | 629 | 9.565 | −2.835 | 20.555 | 1.00 | 24.56 |
| ATOM | 753 | CB | LEU | A | 629 | 9.338 | −1.737 | 19.521 | 1.00 | 24.39 |
| ATOM | 754 | CG | LEU | A | 629 | 8.155 | −1.980 | 18.582 | 1.00 | 25.70 |
| ATOM | 755 | CD1 | LEU | A | 629 | 7.906 | −0.753 | 17.734 | 1.00 | 25.30 |
| ATOM | 756 | CD2 | LEU | A | 629 | 8.446 | −3.200 | 17.711 | 1.00 | 26.08 |
| ATOM | 757 | C | LEU | A | 629 | 10.897 | −2.608 | 21.256 | 1.00 | 25.12 |
| ATOM | 758 | O | LEU | A | 629 | 11.898 | −3.243 | 20.920 | 1.00 | 25.35 |
| ATOM | 759 | N | LYS | A | 630 | 10.907 | −1.705 | 22.234 | 1.00 | 32.90 |
| ATOM | 760 | CA | LYS | A | 630 | 12.126 | −1.394 | 22.981 | 1.00 | 33.87 |
| ATOM | 761 | CB | LYS | A | 630 | 12.033 | 0.012 | 23.573 | 1.00 | 31.73 |
| ATOM | 762 | CG | LYS | A | 630 | 12.137 | 1.116 | 22.544 | 1.00 | 31.51 |
| ATOM | 763 | CD | LYS | A | 630 | 13.494 | 1.089 | 21.868 | 1.00 | 32.17 |
| ATOM | 764 | CE | LYS | A | 630 | 13.682 | 2.292 | 20.966 | 1.00 | 31.52 |
| ATOM | 765 | NZ | LYS | A | 630 | 14.984 | 2.243 | 20.254 | 1.00 | 31.88 |
| ATOM | 766 | C | LYS | A | 630 | 12.416 | −2.394 | 24.096 | 1.00 | 34.55 |
| ATOM | 767 | O | LYS | A | 630 | 13.176 | −3.347 | 23.906 | 1.00 | 35.07 |
| ATOM | 768 | N | ALA | A | 631 | 11.814 | −2.168 | 25.260 | 1.00 | 30.54 |
| ATOM | 769 | CA | ALA | A | 631 | 12.005 | −3.047 | 26.406 | 1.00 | 30.93 |
| ATOM | 770 | CB | ALA | A | 631 | 11.175 | −2.548 | 27.590 | 1.00 | 31.25 |
| ATOM | 771 | C | ALA | A | 631 | 11.630 | −4.492 | 26.070 | 1.00 | 30.76 |
| ATOM | 772 | O | ALA | A | 631 | 12.270 | −5.432 | 26.538 | 1.00 | 30.38 |
| ATOM | 773 | N | GLY | A | 632 | 10.588 | −4.660 | 25.257 | 1.00 | 30.21 |
| ATOM | 774 | CA | GLY | A | 632 | 10.147 | −5.991 | 24.871 | 1.00 | 30.31 |
| ATOM | 775 | C | GLY | A | 632 | 11.073 | −6.644 | 23.860 | 1.00 | 30.73 |
| ATOM | 776 | O | GLY | A | 632 | 10.975 | −7.843 | 23.592 | 1.00 | 30.32 |
| ATOM | 777 | N | LYS | A | 633 | 11.967 | −5.848 | 23.287 | 1.00 | 31.93 |
| ATOM | 778 | CA | LYS | A | 633 | 12.937 | −6.346 | 22.315 | 1.00 | 32.77 |
| ATOM | 779 | CB | LYS | A | 633 | 13.858 | −7.374 | 22.979 | 1.00 | 44.15 |
| ATOM | 780 | CG | LYS | A | 633 | 14.875 | −6.775 | 23.929 | 1.00 | 44.47 |
| ATOM | 781 | CD | LYS | A | 633 | 15.841 | −7.835 | 24.426 | 1.00 | 44.54 |
| ATOM | 782 | CE | LYS | A | 633 | 17.052 | −7.197 | 25.079 | 1.00 | 44.74 |
| ATOM | 783 | NZ | LYS | A | 633 | 17.770 | −6.316 | 24.114 | 1.00 | 44.90 |
| ATOM | 784 | C | LYS | A | 633 | 12.352 | −6.948 | 21.041 | 1.00 | 32.00 |
| ATOM | 785 | O | LYS | A | 633 | 12.862 | −7.944 | 20.522 | 1.00 | 32.88 |
| ATOM | 786 | N | ILE | A | 634 | 11.292 | −6.338 | 20.527 | 1.00 | 26.60 |
| ATOM | 787 | CA | ILE | A | 634 | 10.670 | −6.818 | 19.305 | 1.00 | 25.39 |
| ATOM | 788 | CB | ILE | A | 634 | 9.124 | −6.695 | 19.400 | 1.00 | 26.41 |
| ATOM | 789 | CG2 | ILE | A | 634 | 8.473 | −7.074 | 18.076 | 1.00 | 27.54 |
| ATOM | 790 | CG1 | ILE | A | 634 | 8.607 | −7.612 | 20.510 | 1.00 | 27.29 |
| ATOM | 791 | CD1 | ILE | A | 634 | 7.113 | −7.543 | 20.725 | 1.00 | 27.31 |
| ATOM | 792 | C | ILE | A | 634 | 11.200 | −6.013 | 18.117 | 1.00 | 25.00 |
| ATOM | 793 | O | ILE | A | 634 | 11.127 | −6.446 | 16.968 | 1.00 | 25.10 |
| ATOM | 794 | N | GLN | A | 635 | 11.770 | −4.850 | 18.409 | 1.00 | 23.96 |
| ATOM | 795 | CA | GLN | A | 635 | 12.305 | −3.975 | 17.377 | 1.00 | 23.37 |
| ATOM | 796 | CB | GLN | A | 635 | 12.904 | −2.716 | 18.010 | 1.00 | 27.98 |
| ATOM | 797 | CG | GLN | A | 635 | 13.476 | −1.740 | 17.002 | 1.00 | 28.57 |
| ATOM | 798 | CD | GLN | A | 635 | 14.111 | −0.516 | 17.645 | 1.00 | 28.17 |
| ATOM | 799 | OE1 | GLN | A | 635 | 14.635 | 0.354 | 16.953 | 1.00 | 29.49 |
| ATOM | 800 | NE2 | GLN | A | 635 | 14.059 | −0.444 | 18.967 | 1.00 | 27.30 |
| ATOM | 801 | C | GLN | A | 635 | 13.368 | −4.672 | 16.535 | 1.00 | 24.49 |
| ATOM | 802 | O | GLN | A | 635 | 13.445 | −4.468 | 15.324 | 1.00 | 24.24 |
| ATOM | 803 | N | ASN | A | 636 | 14.180 | −5.493 | 17.195 | 1.00 | 30.08 |
| ATOM | 804 | CA | ASN | A | 636 | 15.266 | −6.222 | 16.547 | 1.00 | 31.17 |
| ATOM | 805 | CB | ASN | A | 636 | 16.172 | −6.829 | 17.618 | 1.00 | 31.58 |
| ATOM | 806 | CG | ASN | A | 636 | 16.612 | −5.811 | 18.651 | 1.00 | 32.71 |
| ATOM | 807 | OD1 | ASN | A | 636 | 17.739 | −5.309 | 18.607 | 1.00 | 32.53 |
| ATOM | 808 | ND2 | ASN | A | 636 | 15.716 | −5.490 | 19.585 | 1.00 | 31.94 |
| ATOM | 809 | C | ASN | A | 636 | 14.782 | −7.330 | 15.617 | 1.00 | 31.51 |
| ATOM | 810 | O | ASN | A | 636 | 15.553 | −7.853 | 14.809 | 1.00 | 31.97 |
| ATOM | 811 | N | LYS | A | 637 | 13.510 | −7.690 | 15.732 | 1.00 | 28.95 |
| ATOM | 812 | CA | LYS | A | 637 | 12.944 | −8.750 | 14.904 | 1.00 | 28.44 |
| ATOM | 813 | CB | LYS | A | 637 | 11.983 | −9.596 | 15.740 | 1.00 | 31.06 |
| ATOM | 814 | CG | LYS | A | 637 | 12.590 | −10.196 | 16.993 | 1.00 | 30.31 |
| ATOM | 815 | CD | LYS | A | 637 | 11.502 | −10.770 | 17.889 | 1.00 | 30.88 |
| ATOM | 816 | CE | LYS | A | 637 | 12.072 | −11.316 | 19.186 | 1.00 | 31.13 |
| ATOM | 817 | NZ | LYS | A | 637 | 12.956 | −12.494 | 18.960 | 1.00 | 31.42 |
| ATOM | 818 | C | LYS | A | 637 | 12.200 | −8.219 | 13.683 | 1.00 | 28.47 |
| ATOM | 819 | O | LYS | A | 637 | 11.696 | −9.003 | 12.875 | 1.00 | 29.67 |
| ATOM | 820 | N | LEU | A | 638 | 12.137 | −6.898 | 13.539 | 1.00 | 27.41 |
| ATOM | 821 | CA | LEU | A | 638 | 11.401 | −6.297 | 12.427 | 1.00 | 27.76 |
| ATOM | 822 | CB | LEU | A | 638 | 10.187 | −5.546 | 12.977 | 1.00 | 24.26 |
| ATOM | 823 | CG | LEU | A | 638 | 9.349 | −6.293 | 14.016 | 1.00 | 24.56 |
| ATOM | 824 | CD1 | LEU | A | 638 | 8.265 | −5.366 | 14.549 | 1.00 | 25.02 |
| ATOM | 825 | CD2 | LEU | A | 638 | 8.740 | −7.549 | 13.396 | 1.00 | 24.12 |
| ATOM | 826 | C | LEU | A | 638 | 12.203 | −5.348 | 11.543 | 1.00 | 26.64 |
| ATOM | 827 | O | LEU | A | 638 | 13.301 | −4.925 | 11.896 | 1.00 | 28.43 |
| ATOM | 828 | N | THR | A | 639 | 11.630 | −5.011 | 10.389 | 1.00 | 23.44 |
| ATOM | 829 | CA | THR | A | 639 | 12.253 | −4.093 | 9.441 | 1.00 | 22.41 |
| ATOM | 830 | CB | THR | A | 639 | 11.772 | −4.343 | 8.004 | 1.00 | 28.95 |
| ATOM | 831 | OG1 | THR | A | 639 | 10.378 | −4.021 | 7.908 | 1.00 | 28.33 |
| ATOM | 832 | CG2 | THR | A | 639 | 11.982 | −5.794 | 7.611 | 1.00 | 28.40 |
| ATOM | 833 | C | THR | A | 639 | 11.870 | −2.662 | 9.798 | 1.00 | 22.44 |
| ATOM | 834 | O | THR | A | 639 | 10.921 | −2.445 | 10.551 | 1.00 | 23.44 |
| ATOM | 835 | N | ASP | A | 640 | 12.601 | −1.695 | 9.250 | 1.00 | 24.48 |
| ATOM | 836 | CA | ASP | A | 640 | 12.321 | −0.288 | 9.508 | 1.00 | 24.67 |
| ATOM | 837 | CB | ASP | A | 640 | 13.313 | 0.604 | 8.747 | 1.00 | 30.57 |
| ATOM | 838 | CG | ASP | A | 640 | 14.693 | 0.642 | 9.393 | 1.00 | 30.89 |
| ATOM | 839 | OD1 | ASP | A | 640 | 15.634 | 1.146 | 8.743 | 1.00 | 30.85 |
| ATOM | 840 | OD2 | ASP | A | 640 | 14.838 | 0.184 | 10.548 | 1.00 | 30.86 |
| ATOM | 841 | C | ASP | A | 640 | 10.895 | 0.038 | 9.074 | 1.00 | 24.64 |
| ATOM | 842 | O | ASP | A | 640 | 10.177 | 0.761 | 9.765 | 1.00 | 24.23 |
| ATOM | 843 | N | LEU | A | 641 | 10.491 | −0.513 | 7.931 | 1.00 | 24.25 |
| ATOM | 844 | CA | LEU | A | 641 | 9.153 | −0.291 | 7.392 | 1.00 | 23.81 |
| ATOM | 845 | CB | LEU | A | 641 | 9.066 | −0.863 | 5.975 | 1.00 | 25.82 |
| ATOM | 846 | CG | LEU | A | 641 | 9.326 | 0.063 | 4.781 | 1.00 | 27.50 |
| ATOM | 847 | CD1 | LEU | A | 641 | 10.267 | 1.188 | 5.137 | 1.00 | 25.96 |
| ATOM | 848 | CD2 | LEU | A | 641 | 9.858 | −0.767 | 3.629 | 1.00 | 26.21 |
| ATOM | 849 | C | LEU | A | 641 | 8.074 | −0.914 | 8.269 | 1.00 | 23.08 |
| ATOM | 850 | O | LEU | A | 641 | 7.013 | −0.332 | 8.464 | 1.00 | 24.13 |
| ATOM | 851 | N | GLU | A | 642 | 8.336 | −2.110 | 8.778 | 1.00 | 23.58 |
| ATOM | 852 | CA | GLU | A | 642 | 7.371 | −2.771 | 9.639 | 1.00 | 24.03 |
| ATOM | 853 | CB | GLU | A | 642 | 7.847 | −4.188 | 9.966 | 1.00 | 29.79 |
| ATOM | 854 | CG | GLU | A | 642 | 7.607 | −5.150 | 8.816 | 1.00 | 30.52 |
| ATOM | 855 | CD | GLU | A | 642 | 8.276 | −6.492 | 9.004 | 1.00 | 30.88 |
| ATOM | 856 | OE1 | GLU | A | 642 | 7.928 | −7.424 | 8.246 | 1.00 | 30.84 |
| ATOM | 857 | OE2 | GLU | A | 642 | 9.147 | −6.615 | 9.893 | 1.00 | 30.40 |
| ATOM | 858 | C | GLU | A | 642 | 7.166 | −1.947 | 10.911 | 1.00 | 24.17 |
| ATOM | 859 | O | GLU | A | 642 | 6.043 | −1.816 | 11.404 | 1.00 | 24.58 |
| ATOM | 860 | N | ILE | A | 643 | 8.260 | −1.382 | 11.419 | 1.00 | 21.47 |
| ATOM | 861 | CA | ILE | A | 643 | 8.230 | −0.555 | 12.625 | 1.00 | 21.07 |
| ATOM | 862 | CB | ILE | A | 643 | 9.659 | −0.184 | 13.062 | 1.00 | 24.97 |
| ATOM | 863 | CG2 | ILE | A | 643 | 9.614 | 0.833 | 14.195 | 1.00 | 23.30 |
| ATOM | 864 | CG1 | ILE | A | 643 | 10.409 | −1.444 | 13.502 | 1.00 | 23.69 |
| ATOM | 865 | CD1 | ILE | A | 643 | 11.900 | −1.229 | 13.673 | 1.00 | 24.32 |
| ATOM | 866 | C | ILE | A | 643 | 7.440 | 0.727 | 12.350 | 1.00 | 20.73 |
| ATOM | 867 | O | ILE | A | 643 | 6.536 | 1.088 | 13.109 | 1.00 | 20.07 |
| ATOM | 868 | N | LEU | A | 644 | 7.792 | 1.405 | 11.259 | 1.00 | 21.45 |
| ATOM | 869 | CA | LEU | A | 644 | 7.122 | 2.632 | 10.843 | 1.00 | 22.42 |
| ATOM | 870 | CB | LEU | A | 644 | 7.707 | 3.112 | 9.510 | 1.00 | 22.90 |
| ATOM | 871 | CG | LEU | A | 644 | 7.028 | 4.294 | 8.816 | 1.00 | 23.31 |
| ATOM | 872 | CD1 | LEU | A | 644 | 7.162 | 5.546 | 9.670 | 1.00 | 23.35 |
| ATOM | 873 | CD2 | LEU | A | 644 | 7.671 | 4.506 | 7.449 | 1.00 | 22.87 |
| ATOM | 874 | C | LEU | A | 644 | 5.623 | 2.400 | 10.686 | 1.00 | 22.48 |
| ATOM | 875 | O | LEU | A | 644 | 4.809 | 3.178 | 11.183 | 1.00 | 21.76 |
| ATOM | 876 | N | ALA | A | 645 | 5.262 | 1.327 | 9.993 | 1.00 | 19.64 |
| ATOM | 877 | CA | ALA | A | 645 | 3.858 | 1.003 | 9.770 | 1.00 | 19.40 |
| ATOM | 878 | CB | ALA | A | 645 | 3.747 | −0.144 | 8.759 | 1.00 | 19.42 |
| ATOM | 879 | C | ALA | A | 645 | 3.110 | 0.643 | 11.062 | 1.00 | 18.96 |
| ATOM | 880 | O | ALA | A | 645 | 1.946 | 1.016 | 11.238 | 1.00 | 16.29 |
| ATOM | 881 | N | LEU | A | 646 | 3.767 | −0.073 | 11.970 | 1.00 | 15.67 |
| ATOM | 882 | CA | LEU | A | 646 | 3.113 | −0.458 | 13.218 | 1.00 | 17.59 |
| ATOM | 883 | CB | LEU | A | 646 | 3.972 | −1.464 | 13.982 | 1.00 | 21.09 |
| ATOM | 884 | CG | LEU | A | 646 | 4.011 | −2.880 | 13.401 | 1.00 | 20.20 |
| ATOM | 885 | CD1 | LEU | A | 646 | 5.105 | −3.678 | 14.102 | 1.00 | 20.69 |
| ATOM | 886 | CD2 | LEU | A | 646 | 2.654 | −3.552 | 13.561 | 1.00 | 21.23 |
| ATOM | 887 | C | LEU | A | 646 | 2.826 | 0.750 | 14.112 | 1.00 | 16.19 |
| ATOM | 888 | O | LEU | A | 646 | 1.770 | 0.824 | 14.748 | 1.00 | 17.93 |
| ATOM | 889 | N | LEU | A | 647 | 3.767 | 1.687 | 14.168 | 1.00 | 20.47 |
| ATOM | 890 | CA | LEU | A | 647 | 3.578 | 2.875 | 15.000 | 1.00 | 18.59 |
| ATOM | 891 | CB | LEU | A | 647 | 4.874 | 3.682 | 15.097 | 1.00 | 19.99 |
| ATOM | 892 | CG | LEU | A | 647 | 4.819 | 4.890 | 16.042 | 1.00 | 19.72 |
| ATOM | 893 | CD1 | LEU | A | 647 | 4.422 | 4.432 | 17.445 | 1.00 | 21.01 |
| ATOM | 894 | CD2 | LEU | A | 647 | 6.168 | 5.563 | 16.078 | 1.00 | 21.38 |
| ATOM | 895 | C | LEU | A | 647 | 2.447 | 3.735 | 14.436 | 1.00 | 19.57 |
| ATOM | 896 | O | LEU | A | 647 | 1.583 | 4.199 | 15.183 | 1.00 | 19.14 |
| ATOM | 897 | N | ILE | A | 648 | 2.447 | 3.935 | 13.120 | 1.00 | 17.04 |
| ATOM | 898 | CA | ILE | A | 648 | 1.393 | 4.714 | 12.475 | 1.00 | 16.34 |
| ATOM | 899 | CB | ILE | A | 648 | 1.671 | 4.884 | 10.962 | 1.00 | 16.27 |
| ATOM | 900 | CG2 | ILE | A | 648 | 0.428 | 5.441 | 10.251 | 1.00 | 17.73 |
| ATOM | 901 | CG1 | ILE | A | 648 | 2.895 | 5.787 | 10.770 | 1.00 | 18.41 |
| ATOM | 902 | CD1 | ILE | A | 648 | 3.350 | 5.921 | 9.339 | 1.00 | 17.00 |
| ATOM | 903 | C | ILE | A | 648 | 0.060 | 3.999 | 12.674 | 1.00 | 17.17 |
| ATOM | 904 | O | ILE | A | 648 | −0.946 | 4.620 | 13.027 | 1.00 | 16.88 |
| ATOM | 905 | N | ALA | A | 649 | 0.056 | 2.684 | 12.477 | 1.00 | 17.97 |
| ATOM | 906 | CA | ALA | A | 649 | −1.171 | 1.912 | 12.634 | 1.00 | 18.14 |
| ATOM | 907 | CB | ALA | A | 649 | −0.927 | 0.460 | 12.228 | 1.00 | 19.75 |
| ATOM | 908 | C | ALA | A | 649 | −1.724 | 1.977 | 14.060 | 1.00 | 17.69 |
| ATOM | 909 | O | ALA | A | 649 | −2.928 | 2.180 | 14.266 | 1.00 | 17.10 |
| ATOM | 910 | N | ALA | A | 650 | −0.850 | 1.802 | 15.047 | 1.00 | 16.14 |
| ATOM | 911 | CA | ALA | A | 650 | −1.276 | 1.846 | 16.438 | 1.00 | 15.98 |
| ATOM | 912 | CB | ALA | A | 650 | −0.070 | 1.703 | 17.365 | 1.00 | 18.40 |
| ATOM | 913 | C | ALA | A | 650 | −1.987 | 3.175 | 16.708 | 1.00 | 16.55 |
| ATOM | 914 | O | ALA | A | 650 | −3.049 | 3.214 | 17.327 | 1.00 | 17.85 |
| ATOM | 915 | N | LEU | A | 651 | −1.401 | 4.259 | 16.226 | 1.00 | 17.09 |
| ATOM | 916 | CA | LEU | A | 651 | −1.974 | 5.589 | 16.438 | 1.00 | 17.07 |
| ATOM | 917 | CB | LEU | A | 651 | −0.956 | 6.659 | 16.056 | 1.00 | 20.77 |
| ATOM | 918 | CG | LEU | A | 651 | 0.304 | 6.766 | 16.906 | 1.00 | 20.44 |
| ATOM | 919 | CD1 | LEU | A | 651 | 1.326 | 7.596 | 16.156 | 1.00 | 20.57 |
| ATOM | 920 | CD2 | LEU | A | 651 | −0.036 | 7.402 | 18.255 | 1.00 | 20.14 |
| ATOM | 921 | C | LEU | A | 651 | −3.246 | 5.846 | 15.648 | 1.00 | 17.89 |
| ATOM | 922 | O | LEU | A | 651 | −4.133 | 6.569 | 16.102 | 1.00 | 17.40 |
| ATOM | 923 | N | SER | A | 652 | −3.335 | 5.248 | 14.465 | 1.00 | 14.66 |
| ATOM | 924 | CA | SER | A | 652 | −4.484 | 5.470 | 13.591 | 1.00 | 16.84 |
| ATOM | 925 | CB | SER | A | 652 | −4.013 | 5.620 | 12.141 | 1.00 | 19.10 |
| ATOM | 926 | OG | SER | A | 652 | −2.948 | 6.535 | 12.014 | 1.00 | 18.78 |
| ATOM | 927 | C | SER | A | 652 | −5.549 | 4.386 | 13.596 | 1.00 | 17.02 |
| ATOM | 928 | O | SER | A | 652 | −6.593 | 4.574 | 12.987 | 1.00 | 16.48 |
| ATOM | 929 | N | HIS | A | 653 | −5.302 | 3.280 | 14.292 | 1.00 | 19.51 |
| ATOM | 930 | CA | HIS | A | 653 | −6.219 | 2.143 | 14.241 | 1.00 | 18.51 |
| ATOM | 931 | CB | HIS | A | 653 | −5.607 | 0.939 | 14.972 | 1.00 | 15.82 |
| ATOM | 932 | CG | HIS | A | 653 | −5.944 | 0.882 | 16.427 | 1.00 | 16.43 |
| ATOM | 933 | CD2 | HIS | A | 653 | −6.947 | 0.252 | 17.079 | 1.00 | 15.65 |
| ATOM | 934 | ND1 | HIS | A | 653 | −5.237 | 1.574 | 17.385 | 1.00 | 14.87 |
| ATOM | 935 | CE1 | HIS | A | 653 | −5.794 | 1.376 | 18.567 | 1.00 | 15.10 |
| ATOM | 936 | NE2 | HIS | A | 653 | −6.838 | 0.580 | 18.406 | 1.00 | 15.73 |
| ATOM | 937 | C | HIS | A | 653 | −7.681 | 2.284 | 14.652 | 1.00 | 19.28 |
| ATOM | 938 | O | HIS | A | 653 | −8.486 | 1.441 | 14.289 | 1.00 | 17.55 |
| ATOM | 939 | N | ASP | A | 654 | −8.039 | 3.329 | 15.395 | 1.00 | 14.03 |
| ATOM | 940 | CA | ASP | A | 654 | −9.434 | 3.505 | 15.811 | 1.00 | 13.68 |
| ATOM | 941 | CB | ASP | A | 654 | −9.576 | 3.404 | 17.342 | 1.00 | 17.23 |
| ATOM | 942 | CG | ASP | A | 654 | −9.893 | 1.995 | 17.816 | 1.00 | 16.81 |
| ATOM | 943 | OD1 | ASP | A | 654 | −10.559 | 1.251 | 17.063 | 1.00 | 16.45 |
| ATOM | 944 | OD2 | ASP | A | 654 | −9.510 | 1.647 | 18.953 | 1.00 | 16.54 |
| ATOM | 945 | C | ASP | A | 654 | −9.986 | 4.858 | 15.351 | 1.00 | 14.35 |
| ATOM | 946 | O | ASP | A | 654 | −11.004 | 5.325 | 15.870 | 1.00 | 15.00 |
| ATOM | 947 | N | LEU | A | 655 | −9.342 | 5.461 | 14.353 | 1.00 | 14.26 |
| ATOM | 948 | CA | LEU | A | 655 | −9.745 | 6.777 | 13.838 | 1.00 | 15.05 |
| ATOM | 949 | CB | LEU | A | 655 | −8.976 | 7.071 | 12.542 | 1.00 | 21.81 |
| ATOM | 950 | CG | LEU | A | 655 | −8.028 | 8.263 | 12.357 | 1.00 | 25.53 |
| ATOM | 951 | CD1 | LEU | A | 655 | −7.712 | 8.958 | 13.671 | 1.00 | 22.91 |
| ATOM | 952 | CD2 | LEU | A | 655 | −6.766 | 7.763 | 11.665 | 1.00 | 22.84 |
| ATOM | 953 | C | LEU | A | 655 | −11.257 | 6.941 | 13.591 | 1.00 | 16.32 |
| ATOM | 954 | O | LEU | A | 655 | −11.889 | 6.120 | 12.923 | 1.00 | 15.44 |
| ATOM | 955 | N | ASP | A | 656 | −11.826 | 8.013 | 14.147 | 1.00 | 16.42 |
| ATOM | 956 | CA | ASP | A | 656 | −13.251 | 8.326 | 13.988 | 1.00 | 17.71 |
| ATOM | 957 | CB | ASP | A | 656 | −13.556 | 8.647 | 12.508 | 1.00 | 20.19 |
| ATOM | 958 | CG | ASP | A | 656 | −14.905 | 9.353 | 12.312 | 1.00 | 20.24 |
| ATOM | 959 | OD1 | ASP | A | 656 | −15.243 | 10.252 | 13.110 | 1.00 | 19.07 |
| ATOM | 960 | OD2 | ASP | A | 656 | −15.623 | 9.021 | 11.344 | 1.00 | 19.47 |
| ATOM | 961 | C | ASP | A | 656 | −14.197 | 7.232 | 14.496 | 1.00 | 16.50 |
| ATOM | 962 | O | ASP | A | 656 | −15.295 | 7.060 | 13.971 | 1.00 | 16.42 |
| ATOM | 963 | N | HIS | A | 657 | −13.777 | 6.502 | 15.523 | 1.00 | 16.01 |
| ATOM | 964 | CA | HIS | A | 657 | −14.627 | 5.454 | 16.073 | 1.00 | 15.64 |
| ATOM | 965 | CB | HIS | A | 657 | −13.902 | 4.710 | 17.196 | 1.00 | 18.44 |
| ATOM | 966 | CG | HIS | A | 657 | −14.585 | 3.446 | 17.606 | 1.00 | 19.68 |
| ATOM | 967 | CD2 | HIS | A | 657 | −15.832 | 3.224 | 18.080 | 1.00 | 15.96 |
| ATOM | 968 | ND1 | HIS | A | 657 | −13.979 | 2.209 | 17.524 | 1.00 | 19.05 |
| ATOM | 969 | CE1 | HIS | A | 657 | −14.825 | 1.281 | 17.930 | 1.00 | 16.34 |
| ATOM | 970 | NE2 | HIS | A | 657 | −15.958 | 1.871 | 18.271 | 1.00 | 21.05 |
| ATOM | 971 | C | HIS | A | 657 | −15.893 | 6.126 | 16.611 | 1.00 | 17.49 |
| ATOM | 972 | O | HIS | A | 657 | −15.826 | 7.093 | 17.377 | 1.00 | 16.58 |
| ATOM | 973 | N | PRO | A | 658 | −17.068 | 5.625 | 16.216 | 1.00 | 19.29 |
| ATOM | 974 | CD | PRO | A | 658 | −17.281 | 4.579 | 15.199 | 1.00 | 17.12 |
| ATOM | 975 | CA | PRO | A | 658 | −18.346 | 6.189 | 16.659 | 1.00 | 19.86 |
| ATOM | 976 | CB | PRO | A | 658 | −19.313 | 5.701 | 15.595 | 1.00 | 16.51 |
| ATOM | 977 | CG | PRO | A | 658 | −18.772 | 4.311 | 15.313 | 1.00 | 15.25 |
| ATOM | 978 | C | PRO | A | 658 | −18.789 | 5.780 | 18.058 | 1.00 | 19.74 |
| ATOM | 979 | O | PRO | A | 658 | −19.745 | 6.335 | 18.589 | 1.00 | 20.03 |
| ATOM | 980 | N | GLY | A | 659 | −18.125 | 4.788 | 18.639 | 1.00 | 16.93 |
| ATOM | 981 | CA | GLY | A | 659 | −18.500 | 4.360 | 19.972 | 1.00 | 18.25 |
| ATOM | 982 | C | GLY | A | 659 | −19.458 | 3.189 | 20.003 | 1.00 | 18.14 |
| ATOM | 983 | O | GLY | A | 659 | −19.993 | 2.857 | 21.060 | 1.00 | 17.96 |
| ATOM | 984 | N | VAL | A | 660 | −19.695 | 2.581 | 18.844 | 1.00 | 19.22 |
| ATOM | 985 | CA | VAL | A | 660 | −20.573 | 1.418 | 18.749 | 1.00 | 20.26 |
| ATOM | 986 | CB | VAL | A | 660 | −21.909 | 1.771 | 18.060 | 1.00 | 19.84 |
| ATOM | 987 | CG1 | VAL | A | 660 | −22.697 | 2.730 | 18.941 | 1.00 | 20.64 |
| ATOM | 988 | CG2 | VAL | A | 660 | −21.655 | 2.391 | 16.698 | 1.00 | 20.17 |
| ATOM | 989 | C | VAL | A | 660 | −19.844 | 0.334 | 17.958 | 1.00 | 20.35 |
| ATOM | 990 | O | VAL | A | 660 | −18.953 | 0.630 | 17.160 | 1.00 | 20.64 |
| ATOM | 991 | N | SER | A | 661 | −20.226 | −0.915 | 18.190 | 1.00 | 18.66 |
| ATOM | 992 | CA | SER | A | 661 | −19.605 | −2.069 | 17.541 | 1.00 | 19.40 |
| ATOM | 993 | CB | SER | A | 661 | −20.001 | −3.341 | 18.278 | 1.00 | 23.46 |
| ATOM | 994 | OG | SER | A | 661 | −21.369 | −3.622 | 18.044 | 1.00 | 22.76 |
| ATOM | 995 | C | SER | A | 661 | −19.955 | −2.260 | 16.069 | 1.00 | 19.19 |
| ATOM | 996 | O | SER | A | 661 | −20.857 | −1.614 | 15.539 | 1.00 | 19.47 |
| ATOM | 997 | N | ASN | A | 662 | −19.237 | −3.175 | 15.421 | 1.00 | 20.96 |
| ATOM | 998 | CA | ASN | A | 662 | −19.493 | −3.489 | 14.018 | 1.00 | 21.17 |
| ATOM | 999 | CB | ASN | A | 662 | −18.474 | −4.500 | 13.487 | 1.00 | 20.86 |
| ATOM | 1000 | CG | ASN | A | 662 | −17.189 | −3.844 | 13.053 | 1.00 | 20.37 |
| ATOM | 1001 | OD1 | ASN | A | 662 | −17.200 | −2.702 | 12.606 | 1.00 | 21.25 |
| ATOM | 1002 | ND2 | ASN | A | 662 | −16.078 | −4.566 | 13.158 | 1.00 | 19.75 |
| ATOM | 1003 | C | ASN | A | 662 | −20.894 | −4.065 | 13.895 | 1.00 | 21.10 |
| ATOM | 1004 | O | ASN | A | 662 | −21.651 | −3.713 | 12.985 | 1.00 | 21.05 |
| ATOM | 1005 | N | GLN | A | 663 | −21.242 | −4.941 | 14.831 | 1.00 | 25.13 |
| ATOM | 1006 | CA | GLN | A | 663 | −22.556 | −5.555 | 14.836 | 1.00 | 26.08 |
| ATOM | 1007 | CB | GLN | A | 663 | −22.696 | −6.503 | 16.031 | 1.00 | 35.28 |
| ATOM | 1008 | CG | GLN | A | 663 | −21.884 | −7.790 | 15.904 | 1.00 | 36.63 |
| ATOM | 1009 | CD | GLN | A | 663 | −20.381 | −7.582 | 16.043 | 1.00 | 36.48 |
| ATOM | 1010 | OE1 | GLN | A | 663 | −19.588 | −8.447 | 15.665 | 1.00 | 36.95 |
| ATOM | 1011 | NE2 | GLN | A | 663 | −19.983 | −6.441 | 16.600 | 1.00 | 36.26 |
| ATOM | 1012 | C | GLN | A | 663 | −23.638 | −4.480 | 14.881 | 1.00 | 25.84 |
| ATOM | 1013 | O | GLN | A | 663 | −24.625 | −4.554 | 14.153 | 1.00 | 25.82 |
| ATOM | 1014 | N | PHE | A | 664 | −23.451 | −3.474 | 15.730 | 1.00 | 24.41 |
| ATOM | 1015 | CA | PHE | A | 664 | −24.423 | −2.394 | 15.832 | 1.00 | 25.34 |
| ATOM | 1016 | CB | PHE | A | 664 | −23.991 | −1.391 | 16.906 | 1.00 | 26.39 |
| ATOM | 1017 | CG | PHE | A | 664 | −24.932 | −0.226 | 17.069 | 1.00 | 27.42 |
| ATOM | 1018 | CD1 | PHE | A | 664 | −24.841 | 0.881 | 16.230 | 1.00 | 26.41 |
| ATOM | 1019 | CD2 | PHE | A | 664 | −25.915 | −0.240 | 18.057 | 1.00 | 27.03 |
| ATOM | 1020 | CE1 | PHE | A | 664 | −25.713 | 1.963 | 16.367 | 1.00 | 27.10 |
| ATOM | 1021 | CE2 | PHE | A | 664 | −26.795 | 0.840 | 18.202 | 1.00 | 27.63 |
| ATOM | 1022 | CZ | PHE | A | 664 | −26.687 | 1.943 | 17.350 | 1.00 | 26.59 |
| ATOM | 1023 | C | PHE | A | 664 | −24.577 | −1.697 | 14.476 | 1.00 | 25.16 |
| ATOM | 1024 | O | PHE | A | 664 | −25.691 | −1.485 | 14.006 | 1.00 | 24.58 |
| ATOM | 1025 | N | LEU | A | 665 | −23.454 | −1.361 | 13.847 | 1.00 | 21.05 |
| ATOM | 1026 | CA | LEU | A | 665 | −23.479 | −0.698 | 12.542 | 1.00 | 22.93 |
| ATOM | 1027 | CB | LEU | A | 665 | −22.058 | −0.348 | 12.104 | 1.00 | 22.29 |
| ATOM | 1028 | CG | LEU | A | 665 | −21.328 | 0.745 | 12.893 | 1.00 | 22.66 |
| ATOM | 1029 | CD1 | LEU | A | 665 | −19.914 | 0.890 | 12.347 | 1.00 | 22.68 |
| ATOM | 1030 | CD2 | LEU | A | 665 | −22.069 | 2.064 | 12.776 | 1.00 | 22.45 |
| ATOM | 1031 | C | LEU | A | 665 | −24.147 | −1.563 | 11.471 | 1.00 | 22.68 |
| ATOM | 1032 | O | LEU | A | 665 | −24.748 | −1.053 | 10.520 | 1.00 | 23.83 |
| ATOM | 1033 | N | ILE | A | 666 | −24.031 | −2.874 | 11.618 | 1.00 | 24.12 |
| ATOM | 1034 | CA | ILE | A | 666 | −24.635 | −3.794 | 10.664 | 1.00 | 24.57 |
| ATOM | 1035 | CB | ILE | A | 666 | −24.023 | −5.204 | 10.785 | 1.00 | 26.67 |
| ATOM | 1036 | CG2 | ILE | A | 666 | −24.819 | −6.195 | 9.951 | 1.00 | 26.86 |
| ATOM | 1037 | CG1 | ILE | A | 666 | −22.559 | −5.176 | 10.340 | 1.00 | 25.51 |
| ATOM | 1038 | CD1 | ILE | A | 666 | −21.811 | −6.483 | 10.553 | 1.00 | 26.41 |
| ATOM | 1039 | C | ILE | A | 666 | −26.136 | −3.880 | 10.926 | 1.00 | 26.29 |
| ATOM | 1040 | O | ILE | A | 666 | −26.937 | −3.859 | 9.993 | 1.00 | 26.10 |
| ATOM | 1041 | N | ASN | A | 667 | −26.511 | −3.968 | 12.197 | 1.00 | 31.89 |
| ATOM | 1042 | CA | ASN | A | 667 | −27.920 | −4.070 | 12.563 | 1.00 | 34.09 |
| ATOM | 1043 | CB | ASN | A | 667 | −28.063 | −4.437 | 14.044 | 1.00 | 34.18 |
| ATOM | 1044 | CG | ASN | A | 667 | −27.511 | −5.809 | 14.355 | 1.00 | 33.95 |
| ATOM | 1045 | OD1 | ASN | A | 667 | −27.608 | −6.722 | 13.538 | 1.00 | 34.50 |
| ATOM | 1046 | ND2 | ASN | A | 667 | −26.939 | −5.969 | 15.544 | 1.00 | 34.36 |
| ATOM | 1047 | C | ASN | A | 667 | −28.733 | −2.816 | 12.270 | 1.00 | 34.67 |
| ATOM | 1048 | O | ASN | A | 667 | −29.944 | −2.891 | 12.058 | 1.00 | 34.88 |
| ATOM | 1049 | N | THR | A | 668 | −28.073 | −1.665 | 12.264 | 1.00 | 41.41 |
| ATOM | 1050 | CA | THR | A | 668 | −28.755 | −0.408 | 11.993 | 1.00 | 41.66 |
| ATOM | 1051 | CB | THR | A | 668 | −28.180 | 0.728 | 12.851 | 1.00 | 34.82 |
| ATOM | 1052 | OG1 | THR | A | 668 | −26.774 | 0.855 | 12.598 | 1.00 | 34.37 |
| ATOM | 1053 | CG2 | THR | A | 668 | −28.404 | 0.438 | 14.325 | 1.00 | 34.77 |
| ATOM | 1054 | C | THR | A | 668 | −28.630 | −0.028 | 10.521 | 1.00 | 41.72 |
| ATOM | 1055 | O | THR | A | 668 | −29.012 | 1.070 | 10.121 | 1.00 | 42.66 |
| ATOM | 1056 | N | ASN | A | 669 | −28.096 | −0.945 | 9.720 | 1.00 | 36.50 |
| ATOM | 1057 | CA | ASN | A | 669 | −27.916 | −0.715 | 8.289 | 1.00 | 35.43 |
| ATOM | 1058 | CB | ASN | A | 669 | −29.271 | −0.756 | 7.581 | 1.00 | 39.66 |
| ATOM | 1059 | CG | ASN | A | 669 | −29.861 | −2.147 | 7.553 | 1.00 | 39.15 |
| ATOM | 1060 | OD1 | ASN | A | 669 | −29.314 | −3.048 | 6.919 | 1.00 | 39.61 |
| ATOM | 1061 | ND2 | ASN | A | 669 | −30.974 | −2.334 | 8.247 | 1.00 | 39.97 |
| ATOM | 1062 | C | ASN | A | 669 | −27.217 | 0.603 | 8.002 | 1.00 | 33.95 |
| ATOM | 1063 | O | ASN | A | 669 | −27.676 | 1.406 | 7.187 | 1.00 | 35.22 |
| ATOM | 1064 | N | SER | A | 670 | −26.098 | 0.816 | 8.684 | 1.00 | 24.12 |
| ATOM | 1065 | CA | SER | A | 670 | −25.304 | 2.020 | 8.524 | 1.00 | 24.45 |
| ATOM | 1066 | CB | SER | A | 670 | −24.162 | 2.032 | 9.543 | 1.00 | 36.85 |
| ATOM | 1067 | OG | SER | A | 670 | −23.245 | 3.081 | 9.279 | 1.00 | 38.12 |
| ATOM | 1068 | C | SER | A | 670 | −24.727 | 2.056 | 7.120 | 1.00 | 22.19 |
| ATOM | 1069 | O | SER | A | 670 | −24.529 | 1.013 | 6.498 | 1.00 | 22.80 |
| ATOM | 1070 | N | GLU | A | 671 | −24.461 | 3.261 | 6.626 | 1.00 | 25.15 |
| ATOM | 1071 | CA | GLU | A | 671 | −23.888 | 3.426 | 5.299 | 1.00 | 25.09 |
| ATOM | 1072 | CB | GLU | A | 671 | −23.736 | 4.911 | 4.975 | 1.00 | 30.03 |
| ATOM | 1073 | CG | GLU | A | 671 | −23.322 | 5.198 | 3.547 | 1.00 | 30.00 |
| ATOM | 1074 | CD | GLU | A | 671 | −23.174 | 6.683 | 3.277 | 1.00 | 30.78 |
| ATOM | 1075 | OE1 | GLU | A | 671 | −22.113 | 7.253 | 3.613 | 1.00 | 30.04 |
| ATOM | 1076 | OE2 | GLU | A | 671 | −24.132 | 7.283 | 2.742 | 1.00 | 30.29 |
| ATOM | 1077 | C | GLU | A | 671 | −22.521 | 2.757 | 5.281 | 1.00 | 23.99 |
| ATOM | 1078 | O | GLU | A | 671 | −22.087 | 2.222 | 4.267 | 1.00 | 24.80 |
| ATOM | 1079 | N | LEU | A | 672 | −21.857 | 2.786 | 6.429 | 1.00 | 23.33 |
| ATOM | 1080 | CA | LEU | A | 672 | −20.533 | 2.196 | 6.581 | 1.00 | 21.52 |
| ATOM | 1081 | CB | LEU | A | 672 | −20.010 | 2.512 | 7.984 | 1.00 | 30.40 |
| ATOM | 1082 | CG | LEU | A | 672 | −18.683 | 3.260 | 8.158 | 1.00 | 31.15 |
| ATOM | 1083 | CD1 | LEU | A | 672 | −18.555 | 4.398 | 7.164 | 1.00 | 30.73 |
| ATOM | 1084 | CD2 | LEU | A | 672 | −18.603 | 3.772 | 9.586 | 1.00 | 31.17 |
| ATOM | 1085 | C | LEU | A | 672 | −20.561 | 0.685 | 6.349 | 1.00 | 22.19 |
| ATOM | 1086 | O | LEU | A | 672 | −19.688 | 0.138 | 5.673 | 1.00 | 20.56 |
| ATOM | 1087 | N | ALA | A | 673 | −21.565 | 0.014 | 6.910 | 1.00 | 20.08 |
| ATOM | 1088 | CA | ALA | A | 673 | −21.685 | −1.434 | 6.766 | 1.00 | 20.23 |
| ATOM | 1089 | CB | ALA | A | 673 | −22.827 | −1.958 | 7.639 | 1.00 | 23.81 |
| ATOM | 1090 | C | ALA | A | 673 | −21.934 | −1.794 | 5.310 | 1.00 | 20.27 |
| ATOM | 1091 | O | ALA | A | 673 | −21.446 | −2.808 | 4.821 | 1.00 | 20.29 |
| ATOM | 1092 | N | LEU | A | 674 | −22.714 | −0.970 | 4.620 | 1.00 | 18.83 |
| ATOM | 1093 | CA | LEU | A | 674 | −22.998 | −1.229 | 3.213 | 1.00 | 20.25 |
| ATOM | 1094 | CB | LEU | A | 674 | −24.063 | −0.265 | 2.694 | 1.00 | 26.85 |
| ATOM | 1095 | CG | LEU | A | 674 | −24.330 | −0.385 | 1.189 | 1.00 | 27.73 |
| ATOM | 1096 | CD1 | LEU | A | 674 | −24.803 | −1.793 | 0.849 | 1.00 | 27.05 |
| ATOM | 1097 | CD2 | LEU | A | 674 | −25.364 | 0.646 | 0.779 | 1.00 | 27.39 |
| ATOM | 1098 | C | LEU | A | 674 | −21.740 | −1.082 | 2.363 | 1.00 | 18.25 |
| ATOM | 1099 | O | LEU | A | 674 | −21.454 | −1.915 | 1.500 | 1.00 | 21.42 |
| ATOM | 1100 | N | MET | A | 675 | −20.980 | −0.027 | 2.624 | 1.00 | 22.09 |
| ATOM | 1101 | CA | MET | A | 675 | −19.758 | 0.244 | 1.874 | 1.00 | 23.03 |
| ATOM | 1102 | CB | MET | A | 675 | −19.140 | 1.562 | 2.330 | 1.00 | 41.71 |
| ATOM | 1103 | CG | MET | A | 675 | −20.013 | 2.771 | 2.096 | 1.00 | 42.43 |
| ATOM | 1104 | SD | MET | A | 675 | −19.158 | 4.258 | 2.607 | 1.00 | 43.58 |
| ATOM | 1105 | CE | MET | A | 675 | −18.192 | 4.598 | 1.138 | 1.00 | 42.87 |
| ATOM | 1106 | C | MET | A | 675 | −18.711 | −0.848 | 2.018 | 1.00 | 23.59 |
| ATOM | 1107 | O | MET | A | 675 | −17.961 | −1.130 | 1.079 | 1.00 | 22.39 |
| ATOM | 1108 | N | TYR | A | 676 | −18.662 | −1.463 | 3.193 | 1.00 | 22.64 |
| ATOM | 1109 | CA | TYR | A | 676 | −17.670 | −2.495 | 3.451 | 1.00 | 22.20 |
| ATOM | 1110 | CB | TYR | A | 676 | −16.894 | −2.113 | 4.717 | 1.00 | 21.16 |
| ATOM | 1111 | CG | TYR | A | 676 | −16.197 | −0.767 | 4.574 | 1.00 | 21.36 |
| ATOM | 1112 | CD1 | TYR | A | 676 | −16.385 | 0.249 | 5.513 | 1.00 | 21.19 |
| ATOM | 1113 | CE1 | TYR | A | 676 | −15.774 | 1.501 | 5.352 | 1.00 | 20.94 |
| ATOM | 1114 | CD2 | TYR | A | 676 | −15.378 | −0.503 | 3.475 | 1.00 | 20.92 |
| ATOM | 1115 | CE2 | TYR | A | 676 | −14.760 | 0.739 | 3.307 | 1.00 | 20.99 |
| ATOM | 1116 | CZ | TYR | A | 676 | −14.964 | 1.734 | 4.242 | 1.00 | 21.41 |
| ATOM | 1117 | OH | TYR | A | 676 | −14.374 | 2.969 | 4.059 | 1.00 | 22.70 |
| ATOM | 1118 | C | TYR | A | 676 | −18.206 | −3.926 | 3.531 | 1.00 | 23.64 |
| ATOM | 1119 | O | TYR | A | 676 | −17.519 | −4.824 | 4.019 | 1.00 | 22.37 |
| ATOM | 1120 | N | ASN | A | 677 | −19.421 | −4.129 | 3.025 | 1.00 | 21.83 |
| ATOM | 1121 | CA | ASN | A | 677 | −20.071 | −5.444 | 2.995 | 1.00 | 23.13 |
| ATOM | 1122 | CB | ASN | A | 677 | −19.487 | −6.296 | 1.861 | 1.00 | 56.74 |
| ATOM | 1123 | CG | ASN | A | 677 | −18.007 | −6.571 | 2.033 | 1.00 | 57.80 |
| ATOM | 1124 | OD1 | ASN | A | 677 | −17.594 | −7.241 | 2.979 | 1.00 | 58.17 |
| ATOM | 1125 | ND2 | ASN | A | 677 | −17.197 | −6.052 | 1.116 | 1.00 | 58.15 |
| ATOM | 1126 | C | ASN | A | 677 | −20.019 | −6.223 | 4.311 | 1.00 | 22.92 |
| ATOM | 1127 | O | ASN | A | 677 | −19.726 | −7.425 | 4.332 | 1.00 | 22.51 |
| ATOM | 1128 | N | ASP | A | 678 | −20.317 | −5.521 | 5.398 | 1.00 | 22.36 |
| ATOM | 1129 | CA | ASP | A | 678 | −20.353 | −6.096 | 6.742 | 1.00 | 23.31 |
| ATOM | 1130 | CB | ASP | A | 678 | −21.461 | −7.146 | 6.843 | 1.00 | 24.85 |
| ATOM | 1131 | CG | ASP | A | 678 | −22.824 | −6.604 | 6.466 | 1.00 | 25.27 |
| ATOM | 1132 | OD1 | ASP | A | 678 | −23.031 | −5.376 | 6.536 | 1.00 | 23.89 |
| ATOM | 1133 | OD2 | ASP | A | 678 | −23.699 | −7.420 | 6.111 | 1.00 | 26.42 |
| ATOM | 1134 | C | ASP | A | 678 | −19.070 | −6.725 | 7.261 | 1.00 | 21.81 |
| ATOM | 1135 | O | ASP | A | 678 | −19.084 | −7.336 | 8.332 | 1.00 | 23.49 |
| ATOM | 1136 | N | GLU | A | 679 | −17.972 | −6.578 | 6.533 | 1.00 | 19.14 |
| ATOM | 1137 | CA | GLU | A | 679 | −16.703 | −7.166 | 6.962 | 1.00 | 20.71 |
| ATOM | 1138 | CB | GLU | A | 679 | −15.988 | −7.819 | 5.778 | 1.00 | 46.03 |
| ATOM | 1139 | CG | GLU | A | 679 | −16.640 | −9.084 | 5.259 | 1.00 | 47.01 |
| ATOM | 1140 | CD | GLU | A | 679 | −15.840 | −9.724 | 4.139 | 1.00 | 47.28 |
| ATOM | 1141 | OE1 | GLU | A | 679 | −15.723 | −9.106 | 3.059 | 1.00 | 47.36 |
| ATOM | 1142 | OE2 | GLU | A | 679 | −15.320 | −10.841 | 4.337 | 1.00 | 47.19 |
| ATOM | 1143 | C | GLU | A | 679 | −15.770 | −6.137 | 7.582 | 1.00 | 19.51 |
| ATOM | 1144 | O | GLU | A | 679 | −15.378 | −5.185 | 6.912 | 1.00 | 20.45 |
| ATOM | 1145 | N | SER | A | 680 | −15.407 | −6.345 | 8.848 | 1.00 | 17.83 |
| ATOM | 1146 | CA | SER | A | 680 | −14.505 | −5.442 | 9.574 | 1.00 | 17.23 |
| ATOM | 1147 | CB | SER | A | 680 | −13.043 | −5.747 | 9.224 | 1.00 | 21.15 |
| ATOM | 1148 | OG | SER | A | 680 | −12.707 | −7.098 | 9.511 | 1.00 | 20.61 |
| ATOM | 1149 | C | SER | A | 680 | −14.827 | −3.997 | 9.202 | 1.00 | 16.25 |
| ATOM | 1150 | O | SER | A | 680 | −13.941 | −3.218 | 8.844 | 1.00 | 15.64 |
| ATOM | 1151 | N | VAL | A | 681 | −16.108 | −3.664 | 9.309 | 1.00 | 18.34 |
| ATOM | 1152 | CA | VAL | A | 681 | −16.629 | −2.353 | 8.947 | 1.00 | 16.30 |
| ATOM | 1153 | CB | VAL | A | 681 | −18.115 | −2.245 | 9.327 | 1.00 | 19.28 |
| ATOM | 1154 | CG1 | VAL | A | 681 | −18.674 | −0.895 | 8.881 | 1.00 | 20.72 |
| ATOM | 1155 | CG2 | VAL | A | 681 | −18.894 | −3.397 | 8.665 | 1.00 | 19.24 |
| ATOM | 1156 | C | VAL | A | 681 | −15.864 | −1.173 | 9.529 | 1.00 | 16.04 |
| ATOM | 1157 | O | VAL | A | 681 | −15.356 | −0.347 | 8.790 | 1.00 | 17.05 |
| ATOM | 1158 | N | LEU | A | 681A | −15.782 | −1.110 | 10.852 | 1.00 | 15.07 |
| ATOM | 1159 | CA | LEU | A | 681A | −15.079 | −0.028 | 11.529 | 1.00 | 16.07 |
| ATOM | 1160 | CB | LEU | A | 681A | −15.166 | −0.217 | 13.043 | 1.00 | 22.39 |
| ATOM | 1161 | CG | LEU | A | 681A | −16.520 | 0.073 | 13.680 | 1.00 | 24.40 |
| ATOM | 1162 | CD1 | LEU | A | 681A | −16.571 | −0.536 | 15.076 | 1.00 | 24.68 |
| ATOM | 1163 | CD2 | LEU | A | 681A | −16.732 | 1.573 | 13.710 | 1.00 | 25.32 |
| ATOM | 1164 | C | LEU | A | 681A | −13.613 | 0.027 | 11.125 | 1.00 | 15.73 |
| ATOM | 1165 | O | LEU | A | 681A | −13.070 | 1.089 | 10.848 | 1.00 | 14.66 |
| ATOM | 1166 | N | GLU | A | 682 | −12.975 | −1.134 | 11.078 | 1.00 | 14.20 |
| ATOM | 1167 | CA | GLU | A | 682 | −11.566 | −1.181 | 10.731 | 1.00 | 16.26 |
| ATOM | 1168 | CB | GLU | A | 682 | −11.042 | −2.601 | 10.954 | 1.00 | 17.70 |
| ATOM | 1169 | CG | GLU | A | 682 | −11.075 | −3.043 | 12.424 | 1.00 | 17.27 |
| ATOM | 1170 | CD | GLU | A | 682 | −12.466 | −3.381 | 12.956 | 1.00 | 17.63 |
| ATOM | 1171 | OE1 | GLU | A | 682 | −13.350 | −3.778 | 12.164 | 1.00 | 16.51 |
| ATOM | 1172 | OE2 | GLU | A | 682 | −12.672 | −3.273 | 14.192 | 1.00 | 18.25 |
| ATOM | 1173 | C | GLU | A | 682 | −11.272 | −0.675 | 9.313 | 1.00 | 13.40 |
| ATOM | 1174 | O | GLU | A | 682 | −10.257 | −0.010 | 9.088 | 1.00 | 16.15 |
| ATOM | 1175 | N | HIS | A | 683 | −12.140 | −0.983 | 8.353 | 1.00 | 14.40 |
| ATOM | 1176 | CA | HIS | A | 683 | −11.943 | −0.474 | 7.001 | 1.00 | 15.72 |
| ATOM | 1177 | CB | HIS | A | 683 | −12.955 | −1.084 | 6.030 | 1.00 | 20.22 |
| ATOM | 1178 | CG | HIS | A | 683 | −12.527 | −2.406 | 5.483 | 1.00 | 21.36 |
| ATOM | 1179 | CD2 | HIS | A | 683 | −12.905 | −3.667 | 5.800 | 1.00 | 22.11 |
| ATOM | 1180 | ND1 | HIS | A | 683 | −11.538 | −2.529 | 4.531 | 1.00 | 19.70 |
| ATOM | 1181 | CE1 | HIS | A | 683 | −11.325 | −3.810 | 4.286 | 1.00 | 20.00 |
| ATOM | 1182 | NE2 | HIS | A | 683 | −12.139 | −4.520 | 5.044 | 1.00 | 21.26 |
| ATOM | 1183 | C | HIS | A | 683 | −12.102 | 1.046 | 7.044 | 1.00 | 15.07 |
| ATOM | 1184 | O | HIS | A | 683 | −11.395 | 1.772 | 6.343 | 1.00 | 16.13 |
| ATOM | 1185 | N | HIS | A | 684 | −13.033 | 1.519 | 7.874 | 1.00 | 16.25 |
| ATOM | 1186 | CA | HIS | A | 684 | −13.258 | 2.952 | 8.026 | 1.00 | 17.73 |
| ATOM | 1187 | CB | HIS | A | 684 | −14.505 | 3.216 | 8.878 | 1.00 | 19.40 |
| ATOM | 1188 | CG | HIS | A | 684 | −14.776 | 4.670 | 9.116 | 1.00 | 20.37 |
| ATOM | 1189 | CD2 | HIS | A | 684 | −14.870 | 5.387 | 10.261 | 1.00 | 19.50 |
| ATOM | 1190 | ND1 | HIS | A | 684 | −14.982 | 5.563 | 8.087 | 1.00 | 20.04 |
| ATOM | 1191 | CE1 | HIS | A | 684 | −15.194 | 6.766 | 8.588 | 1.00 | 19.05 |
| ATOM | 1192 | NE2 | HIS | A | 684 | −15.131 | 6.688 | 9.903 | 1.00 | 18.64 |
| ATOM | 1193 | C | HIS | A | 684 | −12.038 | 3.601 | 8.677 | 1.00 | 17.41 |
| ATOM | 1194 | O | HIS | A | 684 | −11.602 | 4.677 | 8.248 | 1.00 | 17.52 |
| ATOM | 1195 | N | HIS | A | 685 | −11.491 | 2.952 | 9.704 | 1.00 | 16.56 |
| ATOM | 1196 | CA | HIS | A | 685 | −10.316 | 3.481 | 10.387 | 1.00 | 17.88 |
| ATOM | 1197 | CB | HIS | A | 685 | −9.886 | 2.568 | 11.553 | 1.00 | 17.97 |
| ATOM | 1198 | CG | HIS | A | 685 | −10.943 | 2.365 | 12.599 | 1.00 | 17.64 |
| ATOM | 1199 | CD2 | HIS | A | 685 | −11.311 | 1.258 | 13.291 | 1.00 | 17.69 |
| ATOM | 1200 | ND1 | HIS | A | 685 | −11.728 | 3.392 | 13.079 | 1.00 | 16.45 |
| ATOM | 1201 | CE1 | HIS | A | 685 | −12.533 | 2.927 | 14.020 | 1.00 | 18.14 |
| ATOM | 1202 | NE2 | HIS | A | 685 | −12.299 | 1.634 | 14.168 | 1.00 | 17.23 |
| ATOM | 1203 | C | HIS | A | 685 | −9.178 | 3.619 | 9.380 | 1.00 | 17.11 |
| ATOM | 1204 | O | HIS | A | 685 | −8.482 | 4.639 | 9.348 | 1.00 | 17.09 |
| ATOM | 1205 | N | PHE | A | 686 | −8.984 | 2.596 | 8.551 | 1.00 | 17.41 |
| ATOM | 1206 | CA | PHE | A | 686 | −7.928 | 2.656 | 7.551 | 1.00 | 17.43 |
| ATOM | 1207 | CB | PHE | A | 686 | −7.766 | 1.325 | 6.810 | 1.00 | 22.00 |
| ATOM | 1208 | CG | PHE | A | 686 | −6.738 | 1.384 | 5.715 | 1.00 | 21.91 |
| ATOM | 1209 | CD1 | PHE | A | 686 | −5.396 | 1.591 | 6.014 | 1.00 | 22.44 |
| ATOM | 1210 | CD2 | PHE | A | 686 | −7.123 | 1.328 | 4.379 | 1.00 | 22.63 |
| ATOM | 1211 | CE1 | PHE | A | 686 | −4.449 | 1.754 | 4.998 | 1.00 | 21.95 |
| ATOM | 1212 | CE2 | PHE | A | 686 | −6.186 | 1.487 | 3.356 | 1.00 | 22.74 |
| ATOM | 1213 | CZ | PHE | A | 686 | −4.847 | 1.704 | 3.668 | 1.00 | 23.18 |
| ATOM | 1214 | C | PHE | A | 686 | −8.183 | 3.760 | 6.531 | 1.00 | 18.51 |
| ATOM | 1215 | O | PHE | A | 686 | −7.250 | 4.436 | 6.104 | 1.00 | 18.35 |
| ATOM | 1216 | N | ASP | A | 687 | −9.439 | 3.934 | 6.130 | 1.00 | 17.80 |
| ATOM | 1217 | CA | ASP | A | 687 | −9.768 | 4.974 | 5.166 | 1.00 | 18.73 |
| ATOM | 1218 | CB | ASP | A | 687 | −11.262 | 4.969 | 4.836 | 1.00 | 27.45 |
| ATOM | 1219 | CG | ASP | A | 687 | −11.661 | 6.129 | 3.932 | 1.00 | 29.42 |
| ATOM | 1220 | OD1 | ASP | A | 687 | −11.162 | 6.190 | 2.789 | 1.00 | 29.29 |
| ATOM | 1221 | OD2 | ASP | A | 687 | −12.465 | 6.984 | 4.366 | 1.00 | 29.84 |
| ATOM | 1222 | C | ASP | A | 687 | −9.389 | 6.329 | 5.751 | 1.00 | 17.84 |
| ATOM | 1223 | O | ASP | A | 687 | −8.793 | 7.164 | 5.070 | 1.00 | 18.06 |
| ATOM | 1224 | N | GLN | A | 688 | −9.726 | 6.534 | 7.020 | 1.00 | 20.35 |
| ATOM | 1225 | CA | GLN | A | 688 | −9.412 | 7.786 | 7.703 | 1.00 | 19.96 |
| ATOM | 1226 | CB | GLN | A | 688 | −10.060 | 7.804 | 9.085 | 1.00 | 24.85 |
| ATOM | 1227 | CG | GLN | A | 688 | −11.577 | 7.795 | 9.021 | 1.00 | 25.62 |
| ATOM | 1228 | CD | GLN | A | 688 | −12.120 | 9.081 | 8.440 | 1.00 | 26.01 |
| ATOM | 1229 | OE1 | GLN | A | 688 | −12.052 | 10.129 | 9.076 | 1.00 | 27.97 |
| ATOM | 1230 | NE2 | GLN | A | 688 | −12.647 | 9.012 | 7.221 | 1.00 | 27.26 |
| ATOM | 1231 | C | GLN | A | 688 | −7.911 | 7.969 | 7.831 | 1.00 | 20.01 |
| ATOM | 1232 | O | GLN | A | 688 | −7.406 | 9.077 | 7.689 | 1.00 | 19.66 |
| ATOM | 1233 | N | CYS | A | 689 | −7.202 | 6.871 | 8.091 | 1.00 | 18.42 |
| ATOM | 1234 | CA | CYS | A | 689 | −5.746 | 6.902 | 8.229 | 1.00 | 18.66 |
| ATOM | 1235 | CB | CYS | A | 689 | −5.224 | 5.498 | 8.556 | 1.00 | 19.50 |
| ATOM | 1236 | SG | CYS | A | 689 | −3.425 | 5.370 | 8.637 | 1.00 | 16.97 |
| ATOM | 1237 | C | CYS | A | 689 | −5.098 | 7.413 | 6.942 | 1.00 | 18.50 |
| ATOM | 1238 | O | CYS | A | 689 | −4.234 | 8.295 | 6.977 | 1.00 | 19.39 |
| ATOM | 1239 | N | LEU | A | 690 | −5.524 | 6.854 | 5.812 | 1.00 | 19.82 |
| ATOM | 1240 | CA | LEU | A | 690 | −5.006 | 7.235 | 4.497 | 1.00 | 20.55 |
| ATOM | 1241 | CB | LEU | A | 690 | −5.556 | 6.288 | 3.429 | 1.00 | 25.80 |
| ATOM | 1242 | CG | LEU | A | 690 | −4.985 | 6.446 | 2.017 | 1.00 | 26.79 |
| ATOM | 1243 | CD1 | LEU | A | 690 | −3.507 | 6.105 | 2.029 | 1.00 | 27.37 |
| ATOM | 1244 | CD2 | LEU | A | 690 | −5.728 | 5.535 | 1.059 | 1.00 | 27.51 |
| ATOM | 1245 | C | LEU | A | 690 | −5.389 | 8.678 | 4.148 | 1.00 | 19.76 |
| ATOM | 1246 | O | LEU | A | 690 | −4.621 | 9.401 | 3.521 | 1.00 | 21.37 |
| ATOM | 1247 | N | MET | A | 691 | −6.587 | 9.088 | 4.549 | 1.00 | 21.76 |
| ATOM | 1248 | CA | MET | A | 691 | −7.044 | 10.451 | 4.285 | 1.00 | 21.56 |
| ATOM | 1249 | CB | MET | A | 691 | −8.450 | 10.654 | 4.851 | 1.00 | 39.93 |
| ATOM | 1250 | CG | MET | A | 691 | −9.006 | 12.052 | 4.633 | 1.00 | 40.74 |
| ATOM | 1251 | SD | MET | A | 691 | −10.617 | 12.278 | 5.400 | 1.00 | 42.16 |
| ATOM | 1252 | CE | MET | A | 691 | −10.138 | 12.715 | 7.088 | 1.00 | 40.97 |
| ATOM | 1253 | C | MET | A | 691 | −6.079 | 11.451 | 4.928 | 1.00 | 22.42 |
| ATOM | 1254 | O | MET | A | 691 | −5.673 | 12.435 | 4.300 | 1.00 | 21.69 |
| ATOM | 1255 | N | ILE | A | 692 | −5.712 | 11.200 | 6.179 | 1.00 | 20.61 |
| ATOM | 1256 | CA | ILE | A | 692 | −4.799 | 12.094 | 6.878 | 1.00 | 21.43 |
| ATOM | 1257 | CB | ILE | A | 692 | −4.704 | 11.744 | 8.385 | 1.00 | 23.64 |
| ATOM | 1258 | CG2 | ILE | A | 692 | −3.670 | 12.633 | 9.066 | 1.00 | 23.04 |
| ATOM | 1259 | CG1 | ILE | A | 692 | −6.072 | 11.921 | 9.056 | 1.00 | 23.49 |
| ATOM | 1260 | CD1 | ILE | A | 692 | −6.591 | 13.352 | 9.054 | 1.00 | 25.57 |
| ATOM | 1261 | C | ILE | A | 692 | −3.403 | 12.045 | 6.252 | 1.00 | 22.50 |
| ATOM | 1262 | O | ILE | A | 692 | −2.759 | 13.081 | 6.084 | 1.00 | 22.01 |
| ATOM | 1263 | N | LEU | A | 693 | −2.940 | 10.846 | 5.901 | 1.00 | 24.42 |
| ATOM | 1264 | CA | LEU | A | 693 | −1.615 | 10.689 | 5.298 | 1.00 | 25.47 |
| ATOM | 1265 | CB | LEU | A | 693 | −1.288 | 9.203 | 5.087 | 1.00 | 21.69 |
| ATOM | 1266 | CG | LEU | A | 693 | −0.950 | 8.368 | 6.333 | 1.00 | 21.16 |
| ATOM | 1267 | CD1 | LEU | A | 693 | −0.945 | 6.888 | 5.985 | 1.00 | 22.43 |
| ATOM | 1268 | CD2 | LEU | A | 693 | 0.411 | 8.794 | 6.887 | 1.00 | 21.71 |
| ATOM | 1269 | C | LEU | A | 693 | −1.499 | 11.437 | 3.972 | 1.00 | 25.25 |
| ATOM | 1270 | O | LEU | A | 693 | −0.414 | 11.883 | 3.601 | 1.00 | 26.01 |
| ATOM | 1271 | N | ASN | A | 694 | −2.614 | 11.579 | 3.261 | 1.00 | 23.58 |
| ATOM | 1272 | CA | ASN | A | 694 | −2.610 | 12.284 | 1.981 | 1.00 | 22.99 |
| ATOM | 1273 | CB | ASN | A | 694 | −3.613 | 11.656 | 1.005 | 1.00 | 26.66 |
| ATOM | 1274 | CG | ASN | A | 694 | −3.178 | 10.294 | 0.514 | 1.00 | 26.69 |
| ATOM | 1275 | OD1 | ASN | A | 694 | −2.009 | 10.078 | 0.209 | 1.00 | 27.10 |
| ATOM | 1276 | ND2 | ASN | A | 694 | −4.127 | 9.369 | 0.413 | 1.00 | 27.29 |
| ATOM | 1277 | C | ASN | A | 694 | −2.949 | 13.763 | 2.132 | 1.00 | 23.59 |
| ATOM | 1278 | O | ASN | A | 694 | −2.913 | 14.508 | 1.155 | 1.00 | 24.38 |
| ATOM | 1279 | N | SER | A | 695 | −3.285 | 14.181 | 3.348 | 1.00 | 24.35 |
| ATOM | 1280 | CA | SER | A | 695 | −3.644 | 15.572 | 3.596 | 1.00 | 24.87 |
| ATOM | 1281 | CB | SER | A | 695 | −4.279 | 15.727 | 4.980 | 1.00 | 30.85 |
| ATOM | 1282 | OG | SER | A | 695 | −5.570 | 15.146 | 5.009 | 1.00 | 31.53 |
| ATOM | 1283 | C | SER | A | 695 | −2.431 | 16.475 | 3.496 | 1.00 | 25.45 |
| ATOM | 1284 | O | SER | A | 695 | −1.399 | 16.213 | 4.108 | 1.00 | 25.07 |
| ATOM | 1285 | N | PRO | A | 696 | −2.544 | 17.558 | 2.717 | 1.00 | 28.09 |
| ATOM | 1286 | CD | PRO | A | 696 | −3.728 | 18.069 | 2.009 | 1.00 | 32.92 |
| ATOM | 1287 | CA | PRO | A | 696 | −1.410 | 18.471 | 2.579 | 1.00 | 28.93 |
| ATOM | 1288 | CB | PRO | A | 696 | −1.978 | 19.603 | 1.718 | 1.00 | 32.61 |
| ATOM | 1289 | CG | PRO | A | 696 | −3.460 | 19.552 | 1.991 | 1.00 | 33.55 |
| ATOM | 1290 | C | PRO | A | 696 | −0.900 | 18.944 | 3.940 | 1.00 | 28.17 |
| ATOM | 1291 | O | PRO | A | 696 | −1.686 | 19.310 | 4.818 | 1.00 | 27.52 |
| ATOM | 1292 | N | GLY | A | 697 | 0.419 | 18.913 | 4.104 | 1.00 | 27.38 |
| ATOM | 1293 | CA | GLY | A | 697 | 1.035 | 19.336 | 5.347 | 1.00 | 27.26 |
| ATOM | 1294 | C | GLY | A | 697 | 1.106 | 18.239 | 6.391 | 1.00 | 27.36 |
| ATOM | 1295 | O | GLY | A | 697 | 1.688 | 18.430 | 7.460 | 1.00 | 27.28 |
| ATOM | 1296 | N | ASN | A | 698 | 0.523 | 17.084 | 6.080 | 1.00 | 24.05 |
| ATOM | 1297 | CA | ASN | A | 698 | 0.506 | 15.951 | 7.006 | 1.00 | 24.41 |
| ATOM | 1298 | CB | ASN | A | 698 | −0.937 | 15.592 | 7.379 | 1.00 | 26.46 |
| ATOM | 1299 | CG | ASN | A | 698 | −1.624 | 16.668 | 8.190 | 1.00 | 26.12 |
| ATOM | 1300 | OD1 | ASN | A | 698 | −2.038 | 17.702 | 7.664 | 1.00 | 27.64 |
| ATOM | 1301 | ND2 | ASN | A | 698 | −1.749 | 16.428 | 9.486 | 1.00 | 26.42 |
| ATOM | 1302 | C | ASN | A | 698 | 1.165 | 14.698 | 6.436 | 1.00 | 24.62 |
| ATOM | 1303 | O | ASN | A | 698 | 1.157 | 13.643 | 7.076 | 1.00 | 22.80 |
| ATOM | 1304 | N | GLN | A | 699 | 1.734 | 14.812 | 5.240 | 1.00 | 24.05 |
| ATOM | 1305 | CA | GLN | A | 699 | 2.348 | 13.669 | 4.572 | 1.00 | 24.71 |
| ATOM | 1306 | CB | GLN | A | 699 | 2.486 | 13.982 | 3.083 | 1.00 | 37.81 |
| ATOM | 1307 | CG | GLN | A | 699 | 1.165 | 14.399 | 2.451 | 1.00 | 38.52 |
| ATOM | 1308 | CD | GLN | A | 699 | 1.324 | 14.910 | 1.036 | 1.00 | 38.73 |
| ATOM | 1309 | OE1 | GLN | A | 699 | 1.768 | 14.184 | 0.148 | 1.00 | 39.77 |
| ATOM | 1310 | NE2 | GLN | A | 699 | 0.964 | 16.170 | 0.819 | 1.00 | 38.87 |
| ATOM | 1311 | C | GLN | A | 699 | 3.687 | 13.229 | 5.151 | 1.00 | 23.74 |
| ATOM | 1312 | O | GLN | A | 699 | 4.739 | 13.494 | 4.573 | 1.00 | 24.82 |
| ATOM | 1313 | N | ILE | A | 700 | 3.639 | 12.530 | 6.284 | 1.00 | 23.85 |
| ATOM | 1314 | CA | ILE | A | 700 | 4.853 | 12.058 | 6.938 | 1.00 | 23.88 |
| ATOM | 1315 | CB | ILE | A | 700 | 4.572 | 11.572 | 8.374 | 1.00 | 25.21 |
| ATOM | 1316 | CG2 | ILE | A | 700 | 4.101 | 12.736 | 9.225 | 1.00 | 24.98 |
| ATOM | 1317 | CG1 | ILE | A | 700 | 3.528 | 10.454 | 8.363 | 1.00 | 25.64 |
| ATOM | 1318 | CD1 | ILE | A | 700 | 3.370 | 9.764 | 9.699 | 1.00 | 25.16 |
| ATOM | 1319 | C | ILE | A | 700 | 5.550 | 10.935 | 6.182 | 1.00 | 24.95 |
| ATOM | 1320 | O | ILE | A | 700 | 6.662 | 10.540 | 6.546 | 1.00 | 24.94 |
| ATOM | 1321 | N | LEU | A | 701 | 4.904 | 10.426 | 5.135 | 1.00 | 25.36 |
| ATOM | 1322 | CA | LEU | A | 701 | 5.476 | 9.350 | 4.327 | 1.00 | 25.43 |
| ATOM | 1323 | CB | LEU | A | 701 | 4.460 | 8.218 | 4.146 | 1.00 | 25.64 |
| ATOM | 1324 | CG | LEU | A | 701 | 3.976 | 7.487 | 5.401 | 1.00 | 25.01 |
| ATOM | 1325 | CD1 | LEU | A | 701 | 2.975 | 6.403 | 5.008 | 1.00 | 25.21 |
| ATOM | 1326 | CD2 | LEU | A | 701 | 5.161 | 6.879 | 6.124 | 1.00 | 25.48 |
| ATOM | 1327 | C | LEU | A | 701 | 5.904 | 9.859 | 2.951 | 1.00 | 27.13 |
| ATOM | 1328 | O | LEU | A | 701 | 6.280 | 9.075 | 2.082 | 1.00 | 26.39 |
| ATOM | 1329 | N | SER | A | 702 | 5.851 | 11.172 | 2.764 | 1.00 | 36.40 |
| ATOM | 1330 | CA | SER | A | 702 | 6.218 | 11.787 | 1.488 | 1.00 | 38.48 |
| ATOM | 1331 | CB | SER | A | 702 | 6.077 | 13.303 | 1.581 | 1.00 | 34.20 |
| ATOM | 1332 | OG | SER | A | 702 | 7.065 | 13.837 | 2.446 | 1.00 | 34.77 |
| ATOM | 1333 | C | SER | A | 702 | 7.639 | 11.459 | 1.036 | 1.00 | 38.77 |
| ATOM | 1334 | O | SER | A | 702 | 7.915 | 11.380 | −0.162 | 1.00 | 39.14 |
| ATOM | 1335 | N | GLY | A | 703 | 8.537 | 11.278 | 1.997 | 1.00 | 37.92 |
| ATOM | 1336 | CA | GLY | A | 703 | 9.920 | 10.984 | 1.673 | 1.00 | 37.06 |
| ATOM | 1337 | C | GLY | A | 703 | 10.242 | 9.556 | 1.271 | 1.00 | 36.77 |
| ATOM | 1338 | O | GLY | A | 703 | 11.329 | 9.293 | 0.760 | 1.00 | 37.21 |
| ATOM | 1339 | N | LEU | A | 704 | 9.316 | 8.628 | 1.493 | 1.00 | 28.47 |
| ATOM | 1340 | CA | LEU | A | 704 | 9.566 | 7.231 | 1.138 | 1.00 | 27.53 |
| ATOM | 1341 | CB | LEU | A | 704 | 8.446 | 6.321 | 1.655 | 1.00 | 29.58 |
| ATOM | 1342 | CG | LEU | A | 704 | 8.208 | 6.233 | 3.161 | 1.00 | 30.45 |
| ATOM | 1343 | CD1 | LEU | A | 704 | 7.255 | 5.088 | 3.465 | 1.00 | 29.64 |
| ATOM | 1344 | CD2 | LEU | A | 704 | 9.510 | 6.005 | 3.855 | 1.00 | 30.15 |
| ATOM | 1345 | C | LEU | A | 704 | 9.682 | 7.037 | −0.364 | 1.00 | 26.70 |
| ATOM | 1346 | O | LEU | A | 704 | 9.092 | 7.783 | −1.142 | 1.00 | 27.33 |
| ATOM | 1347 | N | SER | A | 705 | 10.450 | 6.028 | −0.762 | 1.00 | 29.04 |
| ATOM | 1348 | CA | SER | A | 705 | 10.606 | 5.704 | −2.171 | 1.00 | 28.79 |
| ATOM | 1349 | CB | SER | A | 705 | 11.734 | 4.690 | −2.369 | 1.00 | 23.35 |
| ATOM | 1350 | OG | SER | A | 705 | 11.393 | 3.441 | −1.802 | 1.00 | 24.99 |
| ATOM | 1351 | C | SER | A | 705 | 9.275 | 5.062 | −2.540 | 1.00 | 29.09 |
| ATOM | 1352 | O | SER | A | 705 | 8.493 | 4.712 | −1.654 | 1.00 | 29.81 |
| ATOM | 1353 | N | ILE | A | 706 | 9.019 | 4.894 | −3.831 | 1.00 | 27.02 |
| ATOM | 1354 | CA | ILE | A | 706 | 7.764 | 4.295 | −4.268 | 1.00 | 26.47 |
| ATOM | 1355 | CB | ILE | A | 706 | 7.700 | 4.211 | −5.809 | 1.00 | 35.41 |
| ATOM | 1356 | CG2 | ILE | A | 706 | 6.441 | 3.472 | −6.241 | 1.00 | 36.09 |
| ATOM | 1357 | CG1 | ILE | A | 706 | 7.728 | 5.622 | −6.402 | 1.00 | 36.23 |
| ATOM | 1358 | CD1 | ILE | A | 706 | 7.821 | 5.658 | −7.917 | 1.00 | 36.02 |
| ATOM | 1359 | C | ILE | A | 706 | 7.561 | 2.902 | −3.681 | 1.00 | 26.18 |
| ATOM | 1360 | O | ILE | A | 706 | 6.487 | 2.584 | −3.175 | 1.00 | 26.02 |
| ATOM | 1361 | N | GLU | A | 707 | 8.594 | 2.071 | −3.736 | 1.00 | 25.11 |
| ATOM | 1362 | CA | GLU | A | 707 | 8.484 | 0.720 | −3.206 | 1.00 | 25.30 |
| ATOM | 1363 | CB | GLU | A | 707 | 9.725 | −0.092 | −3.577 | 1.00 | 46.28 |
| ATOM | 1364 | CG | GLU | A | 707 | 9.775 | −0.446 | −5.051 | 1.00 | 47.07 |
| ATOM | 1365 | CD | GLU | A | 707 | 8.667 | −1.406 | −5.452 | 1.00 | 47.32 |
| ATOM | 1366 | OE1 | GLU | A | 707 | 8.296 | −1.422 | −6.645 | 1.00 | 47.31 |
| ATOM | 1367 | OE2 | GLU | A | 707 | 8.177 | −2.153 | −4.576 | 1.00 | 47.02 |
| ATOM | 1368 | C | GLU | A | 707 | 8.284 | 0.730 | −1.692 | 1.00 | 25.18 |
| ATOM | 1369 | O | GLU | A | 707 | 7.483 | −0.042 | −1.164 | 1.00 | 24.41 |
| ATOM | 1370 | N | GLU | A | 708 | 9.005 | 1.610 | −1.002 | 1.00 | 23.37 |
| ATOM | 1371 | CA | GLU | A | 708 | 8.887 | 1.708 | 0.448 | 1.00 | 24.60 |
| ATOM | 1372 | CB | GLU | A | 708 | 9.904 | 2.704 | 1.011 | 1.00 | 30.84 |
| ATOM | 1373 | CG | GLU | A | 708 | 11.278 | 2.100 | 1.265 | 1.00 | 31.16 |
| ATOM | 1374 | CD | GLU | A | 708 | 12.236 | 3.077 | 1.921 | 1.00 | 31.10 |
| ATOM | 1375 | OE1 | GLU | A | 708 | 13.164 | 2.615 | 2.617 | 1.00 | 31.36 |
| ATOM | 1376 | OE2 | GLU | A | 708 | 12.073 | 4.301 | 1.732 | 1.00 | 31.87 |
| ATOM | 1377 | C | GLU | A | 708 | 7.481 | 2.138 | 0.839 | 1.00 | 24.78 |
| ATOM | 1378 | O | GLU | A | 708 | 6.886 | 1.579 | 1.768 | 1.00 | 23.94 |
| ATOM | 1379 | N | TYR | A | 709 | 6.961 | 3.133 | 0.128 | 1.00 | 23.21 |
| ATOM | 1380 | CA | TYR | A | 709 | 5.619 | 3.644 | 0.390 | 1.00 | 25.29 |
| ATOM | 1381 | CB | TYR | A | 709 | 5.295 | 4.802 | −0.550 | 1.00 | 27.48 |
| ATOM | 1382 | CG | TYR | A | 709 | 3.912 | 5.365 | −0.325 | 1.00 | 28.20 |
| ATOM | 1383 | CD1 | TYR | A | 709 | 3.654 | 6.221 | 0.743 | 1.00 | 28.35 |
| ATOM | 1384 | CE1 | TYR | A | 709 | 2.375 | 6.712 | 0.980 | 1.00 | 28.11 |
| ATOM | 1385 | CD2 | TYR | A | 709 | 2.853 | 5.011 | −1.157 | 1.00 | 29.10 |
| ATOM | 1386 | CE2 | TYR | A | 709 | 1.567 | 5.496 | −0.927 | 1.00 | 29.20 |
| ATOM | 1387 | CZ | TYR | A | 709 | 1.337 | 6.343 | 0.142 | 1.00 | 29.09 |
| ATOM | 1388 | OH | TYR | A | 709 | 0.065 | 6.813 | 0.379 | 1.00 | 28.65 |
| ATOM | 1389 | C | TYR | A | 709 | 4.568 | 2.549 | 0.224 | 1.00 | 25.53 |
| ATOM | 1390 | O | TYR | A | 709 | 3.740 | 2.336 | 1.111 | 1.00 | 24.94 |
| ATOM | 1391 | N | LYS | A | 710 | 4.602 | 1.859 | −0.915 | 1.00 | 25.86 |
| ATOM | 1392 | CA | LYS | A | 710 | 3.658 | 0.778 | −1.191 | 1.00 | 26.20 |
| ATOM | 1393 | CB | LYS | A | 710 | 3.955 | 0.144 | −2.551 | 1.00 | 48.58 |
| ATOM | 1394 | CG | LYS | A | 710 | 3.417 | −1.276 | −2.683 | 1.00 | 49.43 |
| ATOM | 1395 | CD | LYS | A | 710 | 3.795 | −1.914 | −4.008 | 1.00 | 49.56 |
| ATOM | 1396 | CE | LYS | A | 710 | 3.501 | −3.408 | −3.993 | 1.00 | 49.72 |
| ATOM | 1397 | NZ | LYS | A | 710 | 2.086 | −3.707 | −3.633 | 1.00 | 49.51 |
| ATOM | 1398 | C | LYS | A | 710 | 3.689 | −0.309 | −0.123 | 1.00 | 25.97 |
| ATOM | 1399 | O | LYS | A | 710 | 2.640 | −0.743 | 0.356 | 1.00 | 25.66 |
| ATOM | 1400 | N | THR | A | 711 | 4.891 | −0.756 | 0.236 | 1.00 | 24.32 |
| ATOM | 1401 | CA | THR | A | 711 | 5.049 | −1.800 | 1.240 | 1.00 | 23.82 |
| ATOM | 1402 | CB | THR | A | 711 | 6.528 | −2.205 | 1.399 | 1.00 | 33.44 |
| ATOM | 1403 | OG1 | THR | A | 711 | 7.030 | −2.673 | 0.141 | 1.00 | 33.80 |
| ATOM | 1404 | CG2 | THR | A | 711 | 6.669 | −3.307 | 2.441 | 1.00 | 33.11 |
| ATOM | 1405 | C | THR | A | 711 | 4.523 | −1.346 | 2.596 | 1.00 | 23.74 |
| ATOM | 1406 | O | THR | A | 711 | 3.861 | −2.106 | 3.295 | 1.00 | 24.01 |
| ATOM | 1407 | N | THR | A | 712 | 4.813 | −0.100 | 2.951 | 1.00 | 22.39 |
| ATOM | 1408 | CA | THR | A | 712 | 4.379 | 0.449 | 4.227 | 1.00 | 21.87 |
| ATOM | 1409 | CB | THR | A | 712 | 5.003 | 1.831 | 4.468 | 1.00 | 23.06 |
| ATOM | 1410 | OG1 | THR | A | 712 | 6.432 | 1.713 | 4.416 | 1.00 | 24.85 |
| ATOM | 1411 | CG2 | THR | A | 712 | 4.604 | 2.371 | 5.832 | 1.00 | 23.79 |
| ATOM | 1412 | C | THR | A | 712 | 2.859 | 0.548 | 4.330 | 1.00 | 21.27 |
| ATOM | 1413 | O | THR | A | 712 | 2.287 | 0.150 | 5.340 | 1.00 | 22.87 |
| ATOM | 1414 | N | LEU | A | 713 | 2.207 | 1.060 | 3.291 | 1.00 | 23.23 |
| ATOM | 1415 | CA | LEU | A | 713 | 0.751 | 1.189 | 3.314 | 1.00 | 22.85 |
| ATOM | 1416 | CB | LEU | A | 713 | 0.249 | 1.892 | 2.049 | 1.00 | 31.38 |
| ATOM | 1417 | CG | LEU | A | 713 | −0.070 | 3.382 | 2.200 | 1.00 | 32.26 |
| ATOM | 1418 | CD1 | LEU | A | 713 | −1.303 | 3.560 | 3.082 | 1.00 | 32.03 |
| ATOM | 1419 | CD2 | LEU | A | 713 | 1.117 | 4.106 | 2.804 | 1.00 | 32.84 |
| ATOM | 1420 | C | LEU | A | 713 | 0.051 | −0.154 | 3.473 | 1.00 | 22.64 |
| ATOM | 1421 | O | LEU | A | 713 | −0.966 | −0.251 | 4.159 | 1.00 | 22.10 |
| ATOM | 1422 | N | LYS | A | 714 | 0.605 | −1.187 | 2.844 | 1.00 | 20.65 |
| ATOM | 1423 | CA | LYS | A | 714 | 0.054 | −2.534 | 2.919 | 1.00 | 20.74 |
| ATOM | 1424 | CB | LYS | A | 714 | 0.822 | −3.457 | 1.970 | 1.00 | 43.81 |
| ATOM | 1425 | CG | LYS | A | 714 | 0.599 | −4.943 | 2.199 | 1.00 | 44.63 |
| ATOM | 1426 | CD | LYS | A | 714 | 1.492 | −5.772 | 1.278 | 1.00 | 44.93 |
| ATOM | 1427 | CE | LYS | A | 714 | 1.524 | −7.235 | 1.692 | 1.00 | 44.98 |
| ATOM | 1428 | NZ | LYS | A | 714 | 0.178 | −7.868 | 1.647 | 1.00 | 44.80 |
| ATOM | 1429 | C | LYS | A | 714 | 0.165 | −3.051 | 4.348 | 1.00 | 19.68 |
| ATOM | 1430 | O | LYS | A | 714 | −0.755 | −3.693 | 4.865 | 1.00 | 20.74 |
| ATOM | 1431 | N | ILE | A | 715 | 1.300 | −2.777 | 4.984 | 1.00 | 20.42 |
| ATOM | 1432 | CA | ILE | A | 715 | 1.504 | −3.216 | 6.348 | 1.00 | 20.28 |
| ATOM | 1433 | CB | ILE | A | 715 | 2.950 | −2.960 | 6.818 | 1.00 | 24.09 |
| ATOM | 1434 | CG2 | ILE | A | 715 | 3.105 | −3.399 | 8.264 | 1.00 | 24.82 |
| ATOM | 1435 | CG1 | ILE | A | 715 | 3.930 | −3.718 | 5.917 | 1.00 | 25.35 |
| ATOM | 1436 | CD1 | ILE | A | 715 | 5.381 | −3.440 | 6.231 | 1.00 | 24.56 |
| ATOM | 1437 | C | ILE | A | 715 | 0.539 | −2.476 | 7.257 | 1.00 | 19.19 |
| ATOM | 1438 | O | ILE | A | 715 | −0.035 | −3.073 | 8.161 | 1.00 | 19.49 |
| ATOM | 1439 | N | ILE | A | 716 | 0.367 | −1.180 | 7.005 | 1.00 | 19.27 |
| ATOM | 1440 | CA | ILE | A | 716 | −0.546 | −0.361 | 7.800 | 1.00 | 18.10 |
| ATOM | 1441 | CB | ILE | A | 716 | −0.519 | 1.112 | 7.345 | 1.00 | 20.08 |
| ATOM | 1442 | CG2 | ILE | A | 716 | −1.672 | 1.877 | 7.984 | 1.00 | 20.73 |
| ATOM | 1443 | CG1 | ILE | A | 716 | 0.828 | 1.743 | 7.717 | 1.00 | 19.90 |
| ATOM | 1444 | CD1 | ILE | A | 716 | 1.027 | 3.151 | 7.204 | 1.00 | 20.14 |
| ATOM | 1445 | C | ILE | A | 716 | −1.977 | −0.890 | 7.705 | 1.00 | 16.92 |
| ATOM | 1446 | O | ILE | A | 716 | −2.647 | −1.066 | 8.727 | 1.00 | 17.86 |
| ATOM | 1447 | N | LYS | A | 717 | −2.449 | −1.143 | 6.488 | 1.00 | 18.04 |
| ATOM | 1448 | CA | LYS | A | 717 | −3.808 | −1.656 | 6.315 | 1.00 | 19.57 |
| ATOM | 1449 | CB | LYS | A | 717 | −4.150 | −1.839 | 4.834 | 1.00 | 22.85 |
| ATOM | 1450 | CG | LYS | A | 717 | −5.590 | −2.297 | 4.619 | 1.00 | 22.42 |
| ATOM | 1451 | CD | LYS | A | 717 | −5.930 | −2.486 | 3.144 | 1.00 | 23.58 |
| ATOM | 1452 | CE | LYS | A | 717 | −7.357 | −2.986 | 2.980 | 1.00 | 22.98 |
| ATOM | 1453 | NZ | LYS | A | 717 | −7.730 | −3.216 | 1.552 | 1.00 | 24.81 |
| ATOM | 1454 | C | LYS | A | 717 | −3.977 | −2.985 | 7.042 | 1.00 | 19.43 |
| ATOM | 1455 | O | LYS | A | 717 | −4.957 | −3.197 | 7.753 | 1.00 | 18.92 |
| ATOM | 1456 | N | GLN | A | 718 | −3.024 | −3.894 | 6.870 | 1.00 | 19.29 |
| ATOM | 1457 | CA | GLN | A | 718 | −3.136 | −5.173 | 7.552 | 1.00 | 17.73 |
| ATOM | 1458 | CB | GLN | A | 718 | −2.009 | −6.099 | 7.096 | 1.00 | 35.14 |
| ATOM | 1459 | CG | GLN | A | 718 | −2.134 | −6.456 | 5.621 | 1.00 | 35.99 |
| ATOM | 1460 | CD | GLN | A | 718 | −0.956 | −7.249 | 5.095 | 1.00 | 36.88 |
| ATOM | 1461 | OE1 | GLN | A | 718 | −0.973 | −7.718 | 3.956 | 1.00 | 37.13 |
| ATOM | 1462 | NE2 | GLN | A | 718 | 0.076 | −7.396 | 5.915 | 1.00 | 36.21 |
| ATOM | 1463 | C | GLN | A | 718 | −3.144 | −5.031 | 9.073 | 1.00 | 16.21 |
| ATOM | 1464 | O | GLN | A | 718 | −3.899 | −5.725 | 9.764 | 1.00 | 16.17 |
| ATOM | 1465 | N | ALA | A | 719 | −2.326 | −4.117 | 9.585 | 1.00 | 17.17 |
| ATOM | 1466 | CA | ALA | A | 719 | −2.236 | −3.886 | 11.020 | 1.00 | 16.40 |
| ATOM | 1467 | CB | ALA | A | 719 | −1.062 | −2.939 | 11.335 | 1.00 | 18.08 |
| ATOM | 1468 | C | ALA | A | 719 | −3.542 | −3.325 | 11.577 | 1.00 | 15.71 |
| ATOM | 1469 | O | ALA | A | 719 | −3.996 | −3.754 | 12.637 | 1.00 | 15.47 |
| ATOM | 1470 | N | ILE | A | 720 | −4.150 | −2.380 | 10.864 | 1.00 | 15.28 |
| ATOM | 1471 | CA | ILE | A | 720 | −5.402 | −1.802 | 11.330 | 1.00 | 17.34 |
| ATOM | 1472 | CB | ILE | A | 720 | −5.758 | −0.520 | 10.525 | 1.00 | 16.58 |
| ATOM | 1473 | CG2 | ILE | A | 720 | −7.185 | −0.054 | 10.855 | 1.00 | 17.16 |
| ATOM | 1474 | CG1 | ILE | A | 720 | −4.736 | 0.570 | 10.852 | 1.00 | 17.26 |
| ATOM | 1475 | CD1 | ILE | A | 720 | −5.042 | 1.931 | 10.241 | 1.00 | 17.39 |
| ATOM | 1476 | C | ILE | A | 720 | −6.516 | −2.847 | 11.257 | 1.00 | 14.75 |
| ATOM | 1477 | O | ILE | A | 720 | −7.288 | −3.002 | 12.199 | 1.00 | 16.06 |
| ATOM | 1478 | N | LEU | A | 721 | −6.583 | −3.605 | 10.165 | 1.00 | 16.72 |
| ATOM | 1479 | CA | LEU | A | 721 | −7.617 | −4.629 | 10.070 | 1.00 | 17.03 |
| ATOM | 1480 | CB | LEU | A | 721 | −7.616 | −5.282 | 8.684 | 1.00 | 21.51 |
| ATOM | 1481 | CG | LEU | A | 721 | −8.136 | −4.398 | 7.544 | 1.00 | 22.77 |
| ATOM | 1482 | CD1 | LEU | A | 721 | −8.089 | −5.164 | 6.236 | 1.00 | 22.53 |
| ATOM | 1483 | CD2 | LEU | A | 721 | −9.556 | −3.947 | 7.834 | 1.00 | 22.98 |
| ATOM | 1484 | C | LEU | A | 721 | −7.442 | −5.694 | 11.148 | 1.00 | 15.19 |
| ATOM | 1485 | O | LEU | A | 721 | −8.416 | −6.269 | 11.624 | 1.00 | 16.45 |
| ATOM | 1486 | N | ALA | A | 722 | −6.199 | −5.940 | 11.546 | 1.00 | 17.48 |
| ATOM | 1487 | CA | ALA | A | 722 | −5.924 | −6.935 | 12.571 | 1.00 | 17.80 |
| ATOM | 1488 | CB | ALA | A | 722 | −4.431 | −7.113 | 12.735 | 1.00 | 18.14 |
| ATOM | 1489 | C | ALA | A | 722 | −6.547 | −6.557 | 13.905 | 1.00 | 18.00 |
| ATOM | 1490 | O | ALA | A | 722 | −6.764 | −7.420 | 14.750 | 1.00 | 18.41 |
| ATOM | 1491 | N | THR | A | 723 | −6.854 | −5.274 | 14.094 | 1.00 | 15.61 |
| ATOM | 1492 | CA | THR | A | 723 | −7.434 | −4.856 | 15.359 | 1.00 | 15.90 |
| ATOM | 1493 | CB | THR | A | 723 | −7.199 | −3.360 | 15.650 | 1.00 | 16.83 |
| ATOM | 1494 | OG1 | THR | A | 723 | −7.801 | −2.550 | 14.635 | 1.00 | 17.34 |
| ATOM | 1495 | CG2 | THR | A | 723 | −5.701 | −3.084 | 15.726 | 1.00 | 18.33 |
| ATOM | 1496 | C | THR | A | 723 | −8.909 | −5.192 | 15.499 | 1.00 | 16.50 |
| ATOM | 1497 | O | THR | A | 723 | −9.542 | −4.838 | 16.492 | 1.00 | 17.29 |
| ATOM | 1498 | N | ASP | A | 724 | −9.452 | −5.875 | 14.498 | 1.00 | 17.56 |
| ATOM | 1499 | CA | ASP | A | 724 | −10.826 | −6.342 | 14.549 | 1.00 | 18.40 |
| ATOM | 1500 | CB | ASP | A | 724 | −11.304 | −6.762 | 13.157 | 1.00 | 16.11 |
| ATOM | 1501 | CG | ASP | A | 724 | −12.737 | −7.273 | 13.160 | 1.00 | 14.99 |
| ATOM | 1502 | OD1 | ASP | A | 724 | −13.297 | −7.492 | 14.254 | 1.00 | 16.02 |
| ATOM | 1503 | OD2 | ASP | A | 724 | −13.310 | −7.458 | 12.069 | 1.00 | 18.73 |
| ATOM | 1504 | C | ASP | A | 724 | −10.707 | −7.582 | 15.431 | 1.00 | 18.02 |
| ATOM | 1505 | O | ASP | A | 724 | −10.114 | −8.571 | 15.006 | 1.00 | 18.53 |
| ATOM | 1506 | N | LEU | A | 725 | −11.242 | −7.536 | 16.649 | 1.00 | 20.52 |
| ATOM | 1507 | CA | LEU | A | 725 | −11.140 | −8.685 | 17.545 | 1.00 | 21.37 |
| ATOM | 1508 | CB | LEU | A | 725 | −11.851 | −8.407 | 18.873 | 1.00 | 31.10 |
| ATOM | 1509 | CG | LEU | A | 725 | −10.960 | −7.911 | 20.016 | 1.00 | 32.17 |
| ATOM | 1510 | CD1 | LEU | A | 725 | −11.782 | −7.798 | 21.293 | 1.00 | 32.32 |
| ATOM | 1511 | CD2 | LEU | A | 725 | −9.809 | −8.885 | 20.232 | 1.00 | 31.61 |
| ATOM | 1512 | C | LEU | A | 725 | −11.673 | −9.981 | 16.942 | 1.00 | 22.61 |
| ATOM | 1513 | O | LEU | A | 725 | −11.229 | −11.061 | 17.316 | 1.00 | 22.86 |
| ATOM | 1514 | N | ALA | A | 726 | −12.613 | −9.887 | 16.006 | 1.00 | 24.20 |
| ATOM | 1515 | CA | ALA | A | 726 | −13.158 | −11.095 | 15.376 | 1.00 | 26.25 |
| ATOM | 1516 | CB | ALA | A | 726 | −14.375 | −10.746 | 14.520 | 1.00 | 26.36 |
| ATOM | 1517 | C | ALA | A | 726 | −12.093 | −11.782 | 14.520 | 1.00 | 25.26 |
| ATOM | 1518 | O | ALA | A | 726 | −12.043 | −13.015 | 14.431 | 1.00 | 26.23 |
| ATOM | 1519 | N | LEU | A | 727 | −11.253 | −10.981 | 13.879 | 1.00 | 25.84 |
| ATOM | 1520 | CA | LEU | A | 727 | −10.183 | −11.497 | 13.037 | 1.00 | 27.01 |
| ATOM | 1521 | CB | LEU | A | 727 | −9.582 | −10.354 | 12.217 | 1.00 | 27.22 |
| ATOM | 1522 | CG | LEU | A | 727 | −9.355 | −10.566 | 10.718 | 1.00 | 29.26 |
| ATOM | 1523 | CD1 | LEU | A | 727 | −10.607 | −11.167 | 10.073 | 1.00 | 27.54 |
| ATOM | 1524 | CD2 | LEU | A | 727 | −9.011 | −9.229 | 10.069 | 1.00 | 28.24 |
| ATOM | 1525 | C | LEU | A | 727 | −9.123 | −12.118 | 13.946 | 1.00 | 26.83 |
| ATOM | 1526 | O | LEU | A | 727 | −8.441 | −13.077 | 13.575 | 1.00 | 27.75 |
| ATOM | 1527 | N | TYR | A | 728 | −8.982 | −11.566 | 15.144 | 1.00 | 23.72 |
| ATOM | 1528 | CA | TYR | A | 728 | −8.013 | −12.096 | 16.094 | 1.00 | 24.54 |
| ATOM | 1529 | CB | TYR | A | 728 | −7.843 | −11.144 | 17.280 | 1.00 | 26.68 |
| ATOM | 1530 | CG | TYR | A | 728 | −7.128 | −11.784 | 18.440 | 1.00 | 27.14 |
| ATOM | 1531 | CD1 | TYR | A | 728 | −7.828 | −12.208 | 19.568 | 1.00 | 27.32 |
| ATOM | 1532 | CE1 | TYR | A | 728 | −7.183 | −12.878 | 20.602 | 1.00 | 27.41 |
| ATOM | 1533 | CD2 | TYR | A | 728 | −5.762 | −12.040 | 18.377 | 1.00 | 27.43 |
| ATOM | 1534 | CE2 | TYR | A | 728 | −5.111 | −12.709 | 19.400 | 1.00 | 27.27 |
| ATOM | 1535 | CZ | TYR | A | 728 | −5.825 | −13.126 | 20.505 | 1.00 | 27.40 |
| ATOM | 1536 | OH | TYR | A | 728 | −5.178 | −13.810 | 21.507 | 1.00 | 28.59 |
| ATOM | 1537 | C | TYR | A | 728 | −8.483 | −13.462 | 16.593 | 1.00 | 24.46 |
| ATOM | 1538 | O | TYR | A | 728 | −7.713 | −14.425 | 16.643 | 1.00 | 24.91 |
| ATOM | 1539 | N | ILE | A | 729 | −9.753 | −13.536 | 16.968 | 1.00 | 25.84 |
| ATOM | 1540 | CA | ILE | A | 729 | −10.335 | −14.779 | 17.463 | 1.00 | 26.31 |
| ATOM | 1541 | CB | ILE | A | 729 | −11.803 | −14.555 | 17.876 | 1.00 | 28.61 |
| ATOM | 1542 | CG2 | ILE | A | 729 | −12.482 | −15.887 | 18.155 | 1.00 | 28.30 |
| ATOM | 1543 | CG1 | ILE | A | 729 | −11.855 | −13.646 | 19.106 | 1.00 | 27.91 |
| ATOM | 1544 | CD1 | ILE | A | 729 | −13.251 | −13.165 | 19.462 | 1.00 | 28.85 |
| ATOM | 1545 | C | ILE | A | 729 | −10.265 | −15.874 | 16.401 | 1.00 | 28.18 |
| ATOM | 1546 | O | ILE | A | 729 | −10.162 | −17.061 | 16.727 | 1.00 | 28.43 |
| ATOM | 1547 | N | LYS | A | 730 | −10.304 | −15.469 | 15.135 | 1.00 | 32.05 |
| ATOM | 1548 | CA | LYS | A | 730 | −10.265 | −16.402 | 14.008 | 1.00 | 33.23 |
| ATOM | 1549 | CB | LYS | A | 730 | −10.833 | −15.714 | 12.758 | 1.00 | 40.71 |
| ATOM | 1550 | CG | LYS | A | 730 | −10.709 | −16.526 | 11.475 | 1.00 | 40.87 |
| ATOM | 1551 | CD | LYS | A | 730 | −11.146 | −15.727 | 10.251 | 1.00 | 40.83 |
| ATOM | 1552 | CE | LYS | A | 730 | −11.013 | −16.555 | 8.977 | 1.00 | 41.35 |
| ATOM | 1553 | NZ | LYS | A | 730 | −11.478 | −15.810 | 7.772 | 1.00 | 41.21 |
| ATOM | 1554 | C | LYS | A | 730 | −8.865 | −16.938 | 13.697 | 1.00 | 33.75 |
| ATOM | 1555 | O | LYS | A | 730 | −8.688 | −18.137 | 13.457 | 1.00 | 33.39 |
| ATOM | 1556 | N | ARG | A | 731 | −7.876 | −16.050 | 13.703 | 1.00 | 31.85 |
| ATOM | 1557 | CA | ARG | A | 731 | −6.503 | −16.428 | 13.380 | 1.00 | 31.83 |
| ATOM | 1558 | CB | ARG | A | 731 | −5.825 | −15.293 | 12.604 | 1.00 | 43.64 |
| ATOM | 1559 | CG | ARG | A | 731 | −6.375 | −15.070 | 11.205 | 1.00 | 44.14 |
| ATOM | 1560 | CD | ARG | A | 731 | −5.975 | −13.707 | 10.644 | 1.00 | 44.16 |
| ATOM | 1561 | NE | ARG | A | 731 | −4.534 | −13.536 | 10.456 | 1.00 | 44.52 |
| ATOM | 1562 | CZ | ARG | A | 731 | −3.809 | −14.173 | 9.541 | 1.00 | 44.27 |
| ATOM | 1563 | NH1 | ARG | A | 731 | −4.383 | −15.039 | 8.716 | 1.00 | 44.55 |
| ATOM | 1564 | NH2 | ARG | A | 731 | −2.507 | −13.933 | 9.440 | 1.00 | 44.01 |
| ATOM | 1565 | C | ARG | A | 731 | −5.603 | −16.813 | 14.549 | 1.00 | 31.39 |
| ATOM | 1566 | O | ARG | A | 731 | −4.510 | −17.339 | 14.333 | 1.00 | 31.88 |
| ATOM | 1567 | N | ARG | A | 732 | −6.040 | −16.566 | 15.780 | 1.00 | 27.33 |
| ATOM | 1568 | CA | ARG | A | 732 | −5.189 | −16.880 | 16.923 | 1.00 | 27.09 |
| ATOM | 1569 | CB | ARG | A | 732 | −5.783 | −16.315 | 18.218 | 1.00 | 27.95 |
| ATOM | 1570 | CG | ARG | A | 732 | −7.063 | −16.972 | 18.680 | 1.00 | 27.63 |
| ATOM | 1571 | CD | ARG | A | 732 | −7.423 | −16.469 | 20.056 | 1.00 | 27.47 |
| ATOM | 1572 | NE | ARG | A | 732 | −8.557 | −17.178 | 20.632 | 1.00 | 27.81 |
| ATOM | 1573 | CZ | ARG | A | 732 | −8.915 | −17.083 | 21.907 | 1.00 | 28.15 |
| ATOM | 1574 | NH1 | ARG | A | 732 | −8.225 | −16.308 | 22.735 | 1.00 | 28.26 |
| ATOM | 1575 | NH2 | ARG | A | 732 | −9.955 | −17.772 | 22.358 | 1.00 | 29.20 |
| ATOM | 1576 | C | ARG | A | 732 | −4.928 | −18.372 | 17.086 | 1.00 | 26.60 |
| ATOM | 1577 | O | ARG | A | 732 | −3.853 | −18.767 | 17.524 | 1.00 | 26.91 |
| ATOM | 1578 | N | GLY | A | 733 | −5.909 | −19.195 | 16.733 | 1.00 | 28.21 |
| ATOM | 1579 | CA | GLY | A | 733 | −5.741 | −20.633 | 16.860 | 1.00 | 28.94 |
| ATOM | 1580 | C | GLY | A | 733 | −4.533 | −21.138 | 16.097 | 1.00 | 28.70 |
| ATOM | 1581 | O | GLY | A | 733 | −3.785 | −21.991 | 16.582 | 1.00 | 29.01 |
| ATOM | 1582 | N | GLU | A | 734 | −4.344 | −20.609 | 14.895 | 1.00 | 26.13 |
| ATOM | 1583 | CA | GLU | A | 734 | −3.224 | −20.999 | 14.049 | 1.00 | 26.64 |
| ATOM | 1584 | CB | GLU | A | 734 | −3.257 | −20.213 | 12.741 | 1.00 | 32.54 |
| ATOM | 1585 | CG | GLU | A | 734 | −2.289 | −20.720 | 11.697 | 1.00 | 32.73 |
| ATOM | 1586 | CD | GLU | A | 734 | −2.261 | −19.855 | 10.458 | 1.00 | 32.93 |
| ATOM | 1587 | OE1 | GLU | A | 734 | −3.330 | −19.331 | 10.068 | 1.00 | 32.62 |
| ATOM | 1588 | OE2 | GLU | A | 734 | −1.168 | −19.713 | 9.867 | 1.00 | 33.12 |
| ATOM | 1589 | C | GLU | A | 734 | −1.927 | −20.705 | 14.783 | 1.00 | 26.89 |
| ATOM | 1590 | O | GLU | A | 734 | −1.071 | −21.571 | 14.935 | 1.00 | 26.89 |
| ATOM | 1591 | N | PHE | A | 735 | −1.797 | −19.463 | 15.233 | 1.00 | 26.37 |
| ATOM | 1592 | CA | PHE | A | 735 | −0.630 | −18.996 | 15.965 | 1.00 | 26.70 |
| ATOM | 1593 | CB | PHE | A | 735 | −0.911 | −17.569 | 16.455 | 1.00 | 35.10 |
| ATOM | 1594 | CG | PHE | A | 735 | 0.263 | −16.884 | 17.099 | 1.00 | 35.60 |
| ATOM | 1595 | CD1 | PHE | A | 735 | 1.487 | −16.801 | 16.448 | 1.00 | 35.44 |
| ATOM | 1596 | CD2 | PHE | A | 735 | 0.116 | −16.256 | 18.333 | 1.00 | 35.57 |
| ATOM | 1597 | CE1 | PHE | A | 735 | 2.549 | −16.098 | 17.013 | 1.00 | 35.65 |
| ATOM | 1598 | CE2 | PHE | A | 735 | 1.168 | −15.550 | 18.909 | 1.00 | 35.48 |
| ATOM | 1599 | CZ | PHE | A | 735 | 2.388 | −15.469 | 18.247 | 1.00 | 35.90 |
| ATOM | 1600 | C | PHE | A | 735 | −0.309 | −19.919 | 17.148 | 1.00 | 26.06 |
| ATOM | 1601 | O | PHE | A | 735 | 0.819 | −20.387 | 17.297 | 1.00 | 26.34 |
| ATOM | 1602 | N | PHE | A | 736 | −1.313 | −20.186 | 17.978 | 1.00 | 27.83 |
| ATOM | 1603 | CA | PHE | A | 736 | −1.144 | −21.049 | 19.146 | 1.00 | 27.77 |
| ATOM | 1604 | CB | PHE | A | 736 | −2.462 | −21.134 | 19.925 | 1.00 | 26.06 |
| ATOM | 1605 | CG | PHE | A | 736 | −2.927 | −19.810 | 20.492 | 1.00 | 26.21 |
| ATOM | 1606 | CD1 | PHE | A | 736 | −4.239 | −19.659 | 20.948 | 1.00 | 25.79 |
| ATOM | 1607 | CD2 | PHE | A | 736 | −2.059 | −18.727 | 20.587 | 1.00 | 25.60 |
| ATOM | 1608 | CE1 | PHE | A | 736 | −4.676 | −18.450 | 21.489 | 1.00 | 25.76 |
| ATOM | 1609 | CE2 | PHE | A | 736 | −2.490 | −17.508 | 21.130 | 1.00 | 25.65 |
| ATOM | 1610 | CZ | PHE | A | 736 | −3.801 | −17.376 | 21.580 | 1.00 | 25.15 |
| ATOM | 1611 | C | PHE | A | 736 | −0.692 | −22.453 | 18.737 | 1.00 | 27.64 |
| ATOM | 1612 | O | PHE | A | 736 | 0.212 | −23.020 | 19.352 | 1.00 | 28.68 |
| ATOM | 1613 | N | GLU | A | 737 | −1.335 | −23.001 | 17.709 | 1.00 | 26.38 |
| ATOM | 1614 | CA | GLU | A | 737 | −1.015 | −24.335 | 17.187 | 1.00 | 26.87 |
| ATOM | 1615 | CB | GLU | A | 737 | −1.860 | −24.633 | 15.943 | 1.00 | 54.89 |
| ATOM | 1616 | CG | GLU | A | 737 | −3.324 | −24.925 | 16.215 | 1.00 | 55.77 |
| ATOM | 1617 | CD | GLU | A | 737 | −3.541 | −26.319 | 16.765 | 1.00 | 56.24 |
| ATOM | 1618 | OE1 | GLU | A | 737 | −3.171 | −27.293 | 16.074 | 1.00 | 56.16 |
| ATOM | 1619 | OE2 | GLU | A | 737 | −4.081 | −26.440 | 17.885 | 1.00 | 55.95 |
| ATOM | 1620 | C | GLU | A | 737 | 0.458 | −24.431 | 16.809 | 1.00 | 26.60 |
| ATOM | 1621 | O | GLU | A | 737 | 1.158 | −25.385 | 17.181 | 1.00 | 24.87 |
| ATOM | 1622 | N | LEU | A | 738 | 0.919 | −23.446 | 16.047 | 1.00 | 24.73 |
| ATOM | 1623 | CA | LEU | A | 738 | 2.304 | −23.402 | 15.603 | 1.00 | 25.81 |
| ATOM | 1624 | CB | LEU | A | 738 | 2.558 | −22.131 | 14.790 | 1.00 | 23.18 |
| ATOM | 1625 | CG | LEU | A | 738 | 2.098 | −22.127 | 13.326 | 1.00 | 24.63 |
| ATOM | 1626 | CD1 | LEU | A | 738 | 0.728 | −22.761 | 13.172 | 1.00 | 25.88 |
| ATOM | 1627 | CD2 | LEU | A | 738 | 2.093 | −20.699 | 12.816 | 1.00 | 24.42 |
| ATOM | 1628 | C | LEU | A | 738 | 3.271 | −23.459 | 16.771 | 1.00 | 25.22 |
| ATOM | 1629 | O | LEU | A | 738 | 4.259 | −24.190 | 16.730 | 1.00 | 26.20 |
| ATOM | 1630 | N | ILE | A | 739 | 2.992 | −22.676 | 17.809 | 1.00 | 22.89 |
| ATOM | 1631 | CA | ILE | A | 739 | 3.851 | −22.643 | 18.983 | 1.00 | 23.09 |
| ATOM | 1632 | CB | ILE | A | 739 | 3.406 | −21.531 | 19.961 | 1.00 | 29.33 |
| ATOM | 1633 | CG2 | ILE | A | 739 | 4.227 | −21.594 | 21.237 | 1.00 | 29.45 |
| ATOM | 1634 | CG1 | ILE | A | 739 | 3.554 | −20.161 | 19.293 | 1.00 | 29.28 |
| ATOM | 1635 | CD1 | ILE | A | 739 | 4.978 | −19.789 | 18.968 | 1.00 | 29.71 |
| ATOM | 1636 | C | ILE | A | 739 | 3.850 | −23.981 | 19.717 | 1.00 | 23.52 |
| ATOM | 1637 | O | ILE | A | 739 | 4.904 | −24.513 | 20.040 | 1.00 | 24.40 |
| ATOM | 1638 | N | ARG | A | 740 | 2.665 | −24.523 | 19.971 | 1.00 | 25.73 |
| ATOM | 1639 | CA | ARG | A | 740 | 2.545 | −25.790 | 20.683 | 1.00 | 27.35 |
| ATOM | 1640 | CB | ARG | A | 740 | 1.070 | −26.135 | 20.897 | 1.00 | 48.66 |
| ATOM | 1641 | CG | ARG | A | 740 | 0.840 | −27.418 | 21.679 | 1.00 | 49.28 |
| ATOM | 1642 | CD | ARG | A | 740 | −0.625 | −27.566 | 22.043 | 1.00 | 49.58 |
| ATOM | 1643 | NE | ARG | A | 740 | −1.484 | −27.521 | 20.863 | 1.00 | 49.67 |
| ATOM | 1644 | CZ | ARG | A | 740 | −1.504 | −28.454 | 19.916 | 1.00 | 49.81 |
| ATOM | 1645 | NH1 | ARG | A | 740 | −0.709 | −29.514 | 20.008 | 1.00 | 50.00 |
| ATOM | 1646 | NH2 | ARG | A | 740 | −2.317 | −28.330 | 18.876 | 1.00 | 49.79 |
| ATOM | 1647 | C | ARG | A | 740 | 3.241 | −26.950 | 19.975 | 1.00 | 27.41 |
| ATOM | 1648 | O | ARG | A | 740 | 3.784 | −27.845 | 20.628 | 1.00 | 27.24 |
| ATOM | 1649 | N | LYS | A | 741 | 3.230 | −26.935 | 18.646 | 1.00 | 27.03 |
| ATOM | 1650 | CA | LYS | A | 741 | 3.863 | −27.999 | 17.874 | 1.00 | 27.38 |
| ATOM | 1651 | CB | LYS | A | 741 | 3.055 | −28.279 | 16.605 | 1.00 | 33.72 |
| ATOM | 1652 | CG | LYS | A | 741 | 1.670 | −28.843 | 16.885 | 1.00 | 34.15 |
| ATOM | 1653 | CD | LYS | A | 741 | 0.952 | −29.259 | 15.614 | 1.00 | 34.24 |
| ATOM | 1654 | CE | LYS | A | 741 | −0.401 | −29.874 | 15.943 | 1.00 | 34.27 |
| ATOM | 1655 | NZ | LYS | A | 741 | −1.166 | −30.255 | 14.726 | 1.00 | 34.95 |
| ATOM | 1656 | C | LYS | A | 741 | 5.315 | −27.692 | 17.511 | 1.00 | 27.29 |
| ATOM | 1657 | O | LYS | A | 741 | 5.937 | −28.430 | 16.745 | 1.00 | 28.07 |
| ATOM | 1658 | N | ASN | A | 742 | 5.854 | −26.606 | 18.061 | 1.00 | 25.30 |
| ATOM | 1659 | CA | ASN | A | 742 | 7.239 | −26.224 | 17.797 | 1.00 | 25.09 |
| ATOM | 1660 | CB | ASN | A | 742 | 8.189 | −27.297 | 18.332 | 1.00 | 29.88 |
| ATOM | 1661 | CG | ASN | A | 742 | 8.202 | −27.351 | 19.837 | 1.00 | 30.15 |
| ATOM | 1662 | OD1 | ASN | A | 742 | 8.592 | −26.389 | 20.491 | 1.00 | 30.86 |
| ATOM | 1663 | ND2 | ASN | A | 742 | 7.768 | −28.474 | 20.400 | 1.00 | 30.30 |
| ATOM | 1664 | C | ASN | A | 742 | 7.498 | −26.005 | 16.318 | 1.00 | 24.75 |
| ATOM | 1665 | O | ASN | A | 742 | 8.526 | −26.431 | 15.782 | 1.00 | 23.22 |
| ATOM | 1666 | N | GLN | A | 743 | 6.564 | −25.319 | 15.665 | 1.00 | 27.53 |
| ATOM | 1667 | CA | GLN | A | 743 | 6.670 | −25.035 | 14.240 | 1.00 | 28.70 |
| ATOM | 1668 | CB | GLN | A | 743 | 5.451 | −25.592 | 13.495 | 1.00 | 37.55 |
| ATOM | 1669 | CG | GLN | A | 743 | 5.093 | −27.027 | 13.845 | 1.00 | 37.58 |
| ATOM | 1670 | CD | GLN | A | 743 | 3.960 | −27.566 | 12.992 | 1.00 | 38.11 |
| ATOM | 1671 | OE1 | GLN | A | 743 | 2.923 | −26.918 | 12.830 | 1.00 | 38.11 |
| ATOM | 1672 | NE2 | GLN | A | 743 | 4.147 | −28.763 | 12.447 | 1.00 | 37.75 |
| ATOM | 1673 | C | GLN | A | 743 | 6.755 | −23.533 | 13.979 | 1.00 | 28.82 |
| ATOM | 1674 | O | GLN | A | 743 | 6.881 | −23.105 | 12.832 | 1.00 | 28.92 |
| ATOM | 1675 | N | PHE | A | 744 | 6.681 | −22.729 | 15.035 | 1.00 | 28.91 |
| ATOM | 1676 | CA | PHE | A | 744 | 6.743 | −21.284 | 14.850 | 1.00 | 29.47 |
| ATOM | 1677 | CB | PHE | A | 744 | 6.432 | −20.556 | 16.157 | 1.00 | 40.72 |
| ATOM | 1678 | CG | PHE | A | 744 | 6.298 | −19.064 | 15.997 | 1.00 | 40.67 |
| ATOM | 1679 | CD1 | PHE | A | 744 | 5.279 | −18.525 | 15.214 | 1.00 | 41.29 |
| ATOM | 1680 | CD2 | PHE | A | 744 | 7.191 | −18.200 | 16.618 | 1.00 | 41.11 |
| ATOM | 1681 | CE1 | PHE | A | 744 | 5.153 | −17.145 | 15.053 | 1.00 | 41.25 |
| ATOM | 1682 | CE2 | PHE | A | 744 | 7.074 | −16.819 | 16.464 | 1.00 | 41.16 |
| ATOM | 1683 | CZ | PHE | A | 744 | 6.053 | −16.292 | 15.680 | 1.00 | 41.33 |
| ATOM | 1684 | C | PHE | A | 744 | 8.097 | −20.819 | 14.326 | 1.00 | 29.13 |
| ATOM | 1685 | O | PHE | A | 744 | 9.146 | −21.260 | 14.792 | 1.00 | 30.17 |
| ATOM | 1686 | N | ASN | A | 745 | 8.055 | −19.921 | 13.349 | 1.00 | 28.18 |
| ATOM | 1687 | CA | ASN | A | 745 | 9.260 | −19.363 | 12.750 | 1.00 | 29.83 |
| ATOM | 1688 | CB | ASN | A | 745 | 9.795 | −20.272 | 11.648 | 1.00 | 38.97 |
| ATOM | 1689 | CG | ASN | A | 745 | 10.919 | −19.622 | 10.867 | 1.00 | 39.20 |
| ATOM | 1690 | OD1 | ASN | A | 745 | 11.985 | −19.348 | 11.411 | 1.00 | 39.54 |
| ATOM | 1691 | ND2 | ASN | A | 745 | 10.679 | −19.357 | 9.590 | 1.00 | 40.15 |
| ATOM | 1692 | C | ASN | A | 745 | 8.941 | −18.005 | 12.149 | 1.00 | 29.56 |
| ATOM | 1693 | O | ASN | A | 745 | 8.208 | −17.914 | 11.165 | 1.00 | 29.59 |
| ATOM | 1694 | N | LEU | A | 746 | 9.499 | −16.956 | 12.741 | 1.00 | 38.07 |
| ATOM | 1695 | CA | LEU | A | 746 | 9.268 | −15.597 | 12.270 | 1.00 | 39.23 |
| ATOM | 1696 | CB | LEU | A | 746 | 9.822 | −14.591 | 13.280 | 1.00 | 38.00 |
| ATOM | 1697 | CG | LEU | A | 746 | 8.967 | −14.381 | 14.528 | 1.00 | 37.62 |
| ATOM | 1698 | CD1 | LEU | A | 746 | 9.676 | −13.427 | 15.475 | 1.00 | 38.20 |
| ATOM | 1699 | CD2 | LEU | A | 746 | 7.609 | −13.821 | 14.125 | 1.00 | 37.68 |
| ATOM | 1700 | C | LEU | A | 746 | 9.852 | −15.302 | 10.895 | 1.00 | 39.66 |
| ATOM | 1701 | O | LEU | A | 746 | 9.455 | −14.335 | 10.245 | 1.00 | 39.51 |
| ATOM | 1702 | N | GLU | A | 747 | 10.792 | −16.128 | 10.449 | 1.00 | 39.64 |
| ATOM | 1703 | CA | GLU | A | 747 | 11.404 | −15.918 | 9.143 | 1.00 | 39.71 |
| ATOM | 1704 | CB | GLU | A | 747 | 12.605 | −16.845 | 8.965 | 1.00 | 45.59 |
| ATOM | 1705 | CG | GLU | A | 747 | 13.921 | −16.094 | 8.881 | 1.00 | 46.56 |
| ATOM | 1706 | CD | GLU | A | 747 | 15.125 | −16.981 | 9.132 | 1.00 | 46.55 |
| ATOM | 1707 | OE1 | GLU | A | 747 | 15.305 | −17.974 | 8.395 | 1.00 | 46.54 |
| ATOM | 1708 | OE2 | GLU | A | 747 | 15.890 | −16.679 | 10.072 | 1.00 | 46.60 |
| ATOM | 1709 | C | GLU | A | 747 | 10.391 | −16.136 | 8.029 | 1.00 | 38.87 |
| ATOM | 1710 | O | GLU | A | 747 | 10.422 | −15.454 | 7.002 | 1.00 | 39.21 |
| ATOM | 1711 | N | ASP | A | 748 | 9.487 | −17.086 | 8.234 | 1.00 | 32.77 |
| ATOM | 1712 | CA | ASP | A | 748 | 8.455 | −17.363 | 7.248 | 1.00 | 32.31 |
| ATOM | 1713 | CB | ASP | A | 748 | 7.654 | −18.600 | 7.652 | 1.00 | 47.03 |
| ATOM | 1714 | CG | ASP | A | 748 | 6.639 | −19.002 | 6.603 | 1.00 | 47.67 |
| ATOM | 1715 | OD1 | ASP | A | 748 | 5.613 | −18.305 | 6.459 | 1.00 | 47.81 |
| ATOM | 1716 | OD2 | ASP | A | 748 | 6.876 | −20.018 | 5.912 | 1.00 | 48.36 |
| ATOM | 1717 | C | ASP | A | 748 | 7.544 | −16.141 | 7.190 | 1.00 | 32.61 |
| ATOM | 1718 | O | ASP | A | 748 | 6.915 | −15.777 | 8.185 | 1.00 | 30.88 |
| ATOM | 1719 | N | PRO | A | 749 | 7.466 | −15.490 | 6.018 | 1.00 | 39.12 |
| ATOM | 1720 | CD | PRO | A | 749 | 8.049 | −15.930 | 4.739 | 1.00 | 38.16 |
| ATOM | 1721 | CA | PRO | A | 749 | 6.634 | −14.299 | 5.824 | 1.00 | 39.58 |
| ATOM | 1722 | CB | PRO | A | 749 | 6.773 | −14.021 | 4.325 | 1.00 | 38.14 |
| ATOM | 1723 | CG | PRO | A | 749 | 7.069 | −15.367 | 3.745 | 1.00 | 38.10 |
| ATOM | 1724 | C | PRO | A | 749 | 5.176 | −14.417 | 6.274 | 1.00 | 39.62 |
| ATOM | 1725 | O | PRO | A | 749 | 4.601 | −13.447 | 6.770 | 1.00 | 39.95 |
| ATOM | 1726 | N | HIS | A | 750 | 4.571 | −15.588 | 6.108 | 1.00 | 32.50 |
| ATOM | 1727 | CA | HIS | A | 750 | 3.181 | −15.742 | 6.524 | 1.00 | 31.88 |
| ATOM | 1728 | CB | HIS | A | 750 | 2.562 | −17.016 | 5.946 | 1.00 | 30.66 |
| ATOM | 1729 | CG | HIS | A | 750 | 1.149 | −17.240 | 6.384 | 1.00 | 30.76 |
| ATOM | 1730 | CD2 | HIS | A | 750 | −0.008 | −16.667 | 5.981 | 1.00 | 30.83 |
| ATOM | 1731 | ND1 | HIS | A | 750 | 0.815 | −18.097 | 7.410 | 1.00 | 31.54 |
| ATOM | 1732 | CE1 | HIS | A | 750 | −0.488 | −18.041 | 7.621 | 1.00 | 31.23 |
| ATOM | 1733 | NE2 | HIS | A | 750 | −1.011 | −17.180 | 6.768 | 1.00 | 31.56 |
| ATOM | 1734 | C | HIS | A | 750 | 3.055 | −15.761 | 8.042 | 1.00 | 31.66 |
| ATOM | 1735 | O | HIS | A | 750 | 2.146 | −15.148 | 8.604 | 1.00 | 31.17 |
| ATOM | 1736 | N | GLU | A | 751 | 3.957 | −16.470 | 8.711 | 1.00 | 31.26 |
| ATOM | 1737 | CA | GLU | A | 751 | 3.913 | −16.519 | 10.165 | 1.00 | 31.20 |
| ATOM | 1738 | CB | GLU | A | 751 | 4.898 | −17.563 | 10.703 | 1.00 | 33.16 |
| ATOM | 1739 | CG | GLU | A | 751 | 4.444 | −19.000 | 10.455 | 1.00 | 33.17 |
| ATOM | 1740 | CD | GLU | A | 751 | 5.215 | −20.017 | 11.274 | 1.00 | 33.56 |
| ATOM | 1741 | OE1 | GLU | A | 751 | 5.432 | −19.772 | 12.478 | 1.00 | 33.10 |
| ATOM | 1742 | OE2 | GLU | A | 751 | 5.587 | −21.071 | 10.720 | 1.00 | 33.79 |
| ATOM | 1743 | C | GLU | A | 751 | 4.247 | −15.127 | 10.699 | 1.00 | 31.23 |
| ATOM | 1744 | O | GLU | A | 751 | 3.836 | −14.753 | 11.800 | 1.00 | 31.00 |
| ATOM | 1745 | N | LYS | A | 752 | 4.979 | −14.364 | 9.892 | 1.00 | 29.23 |
| ATOM | 1746 | CA | LYS | A | 752 | 5.372 | −13.008 | 10.239 | 1.00 | 29.88 |
| ATOM | 1747 | CB | LYS | A | 752 | 6.292 | −12.439 | 9.154 | 1.00 | 37.79 |
| ATOM | 1748 | CG | LYS | A | 752 | 6.867 | −11.058 | 9.453 | 1.00 | 38.37 |
| ATOM | 1749 | CD | LYS | A | 752 | 7.766 | −11.087 | 10.684 | 1.00 | 38.41 |
| ATOM | 1750 | CE | LYS | A | 752 | 8.526 | −9.783 | 10.861 | 1.00 | 38.61 |
| ATOM | 1751 | NZ | LYS | A | 752 | 9.473 | −9.521 | 9.748 | 1.00 | 38.19 |
| ATOM | 1752 | C | LYS | A | 752 | 4.143 | −12.120 | 10.373 | 1.00 | 29.16 |
| ATOM | 1753 | O | LYS | A | 752 | 3.921 | −11.510 | 11.425 | 1.00 | 29.42 |
| ATOM | 1754 | N | GLU | A | 753 | 3.341 | −12.029 | 9.315 | 1.00 | 30.11 |
| ATOM | 1755 | CA | GLU | A | 753 | 2.156 | −11.191 | 9.402 | 1.00 | 30.60 |
| ATOM | 1756 | CB | GLU | A | 753 | 1.467 | −11.040 | 8.038 | 1.00 | 54.85 |
| ATOM | 1757 | CG | GLU | A | 753 | 1.100 | −12.316 | 7.315 | 1.00 | 55.61 |
| ATOM | 1758 | CD | GLU | A | 753 | 0.501 | −12.034 | 5.942 | 1.00 | 55.82 |
| ATOM | 1759 | OE1 | GLU | A | 753 | 1.195 | −11.420 | 5.102 | 1.00 | 55.58 |
| ATOM | 1760 | OE2 | GLU | A | 753 | −0.663 | −12.420 | 5.705 | 1.00 | 55.80 |
| ATOM | 1761 | C | GLU | A | 753 | 1.201 | −11.736 | 10.458 | 1.00 | 29.96 |
| ATOM | 1762 | O | GLU | A | 753 | 0.476 | −10.973 | 11.094 | 1.00 | 30.36 |
| ATOM | 1763 | N | LEU | A | 754 | 1.215 | −13.050 | 10.668 | 1.00 | 25.09 |
| ATOM | 1764 | CA | LEU | A | 754 | 0.364 | −13.638 | 11.695 | 1.00 | 23.83 |
| ATOM | 1765 | CB | LEU | A | 754 | 0.510 | −15.160 | 11.719 | 1.00 | 25.34 |
| ATOM | 1766 | CG | LEU | A | 754 | −0.242 | −15.857 | 12.852 | 1.00 | 26.46 |
| ATOM | 1767 | CD1 | LEU | A | 754 | −1.730 | −15.558 | 12.759 | 1.00 | 25.32 |
| ATOM | 1768 | CD2 | LEU | A | 754 | 0.010 | −17.364 | 12.767 | 1.00 | 25.75 |
| ATOM | 1769 | C | LEU | A | 754 | 0.812 | −13.064 | 13.039 | 1.00 | 23.90 |
| ATOM | 1770 | O | LEU | A | 754 | −0.011 | −12.728 | 13.896 | 1.00 | 24.10 |
| ATOM | 1771 | N | PHE | A | 755 | 2.126 | −12.953 | 13.215 | 1.00 | 22.27 |
| ATOM | 1772 | CA | PHE | A | 755 | 2.673 | −12.405 | 14.444 | 1.00 | 23.27 |
| ATOM | 1773 | CB | PHE | A | 755 | 4.188 | −12.601 | 14.506 | 1.00 | 27.67 |
| ATOM | 1774 | CG | PHE | A | 755 | 4.811 | −11.977 | 15.715 | 1.00 | 27.49 |
| ATOM | 1775 | CD1 | PHE | A | 755 | 4.559 | −12.492 | 16.981 | 1.00 | 27.28 |
| ATOM | 1776 | CD2 | PHE | A | 755 | 5.589 | −10.834 | 15.596 | 1.00 | 26.66 |
| ATOM | 1777 | CE1 | PHE | A | 755 | 5.070 | −11.872 | 18.117 | 1.00 | 28.85 |
| ATOM | 1778 | CE2 | PHE | A | 755 | 6.108 | −10.200 | 16.727 | 1.00 | 27.67 |
| ATOM | 1779 | CZ | PHE | A | 755 | 5.849 | −10.718 | 17.987 | 1.00 | 27.51 |
| ATOM | 1780 | C | PHE | A | 755 | 2.352 | −10.914 | 14.590 | 1.00 | 23.26 |
| ATOM | 1781 | O | PHE | A | 755 | 1.932 | −10.469 | 15.665 | 1.00 | 22.65 |
| ATOM | 1782 | N | LEU | A | 756 | 2.553 | −10.148 | 13.520 | 1.00 | 21.99 |
| ATOM | 1783 | CA | LEU | A | 756 | 2.274 | −8.712 | 13.552 | 1.00 | 23.22 |
| ATOM | 1784 | CB | LEU | A | 756 | 2.593 | −8.059 | 12.206 | 1.00 | 24.32 |
| ATOM | 1785 | CG | LEU | A | 756 | 4.060 | −7.993 | 11.785 | 1.00 | 25.68 |
| ATOM | 1786 | CD1 | LEU | A | 756 | 4.163 | −7.476 | 10.364 | 1.00 | 24.86 |
| ATOM | 1787 | CD2 | LEU | A | 756 | 4.821 | −7.094 | 12.737 | 1.00 | 25.84 |
| ATOM | 1788 | C | LEU | A | 756 | 0.820 | −8.435 | 13.906 | 1.00 | 22.88 |
| ATOM | 1789 | O | LEU | A | 756 | 0.520 | −7.441 | 14.561 | 1.00 | 22.05 |
| ATOM | 1790 | N | ALA | A | 757 | −0.078 | −9.316 | 13.477 | 1.00 | 22.97 |
| ATOM | 1791 | CA | ALA | A | 757 | −1.498 | −9.152 | 13.770 | 1.00 | 22.24 |
| ATOM | 1792 | CB | ALA | A | 757 | −2.326 | −10.143 | 12.950 | 1.00 | 21.11 |
| ATOM | 1793 | C | ALA | A | 757 | −1.733 | −9.387 | 15.255 | 1.00 | 23.73 |
| ATOM | 1794 | O | ALA | A | 757 | −2.491 | −8.668 | 15.907 | 1.00 | 22.64 |
| ATOM | 1795 | N | MET | A | 758 | −1.080 | −10.407 | 15.794 | 1.00 | 17.51 |
| ATOM | 1796 | CA | MET | A | 758 | −1.234 | −10.717 | 17.198 | 1.00 | 18.28 |
| ATOM | 1797 | CB | MET | A | 758 | −0.581 | −12.072 | 17.503 | 1.00 | 27.85 |
| ATOM | 1798 | CG | MET | A | 758 | −1.181 | −13.255 | 16.720 | 1.00 | 27.03 |
| ATOM | 1799 | SD | MET | A | 758 | −2.826 | −13.794 | 17.241 | 1.00 | 29.72 |
| ATOM | 1800 | CE | MET | A | 758 | −3.778 | −13.473 | 15.747 | 1.00 | 28.03 |
| ATOM | 1801 | C | MET | A | 758 | −0.608 | −9.605 | 18.039 | 1.00 | 17.15 |
| ATOM | 1802 | O | MET | A | 758 | −1.088 | −9.319 | 19.137 | 1.00 | 18.07 |
| ATOM | 1803 | N | LEU | A | 759 | 0.447 | −8.979 | 17.515 | 1.00 | 17.47 |
| ATOM | 1804 | CA | LEU | A | 759 | 1.139 | −7.898 | 18.225 | 1.00 | 19.04 |
| ATOM | 1805 | CB | LEU | A | 759 | 2.418 | −7.482 | 17.491 | 1.00 | 19.37 |
| ATOM | 1806 | CG | LEU | A | 759 | 3.214 | −6.328 | 18.112 | 1.00 | 20.18 |
| ATOM | 1807 | CD1 | LEU | A | 759 | 3.610 | −6.669 | 19.550 | 1.00 | 21.45 |
| ATOM | 1808 | CD2 | LEU | A | 759 | 4.439 | −6.039 | 17.261 | 1.00 | 21.48 |
| ATOM | 1809 | C | LEU | A | 759 | 0.213 | −6.699 | 18.342 | 1.00 | 19.95 |
| ATOM | 1810 | O | LEU | A | 759 | 0.119 | −6.081 | 19.406 | 1.00 | 18.11 |
| ATOM | 1811 | N | MET | A | 760 | −0.454 | −6.367 | 17.240 | 1.00 | 17.24 |
| ATOM | 1812 | CA | MET | A | 760 | −1.393 | −5.240 | 17.251 | 1.00 | 17.90 |
| ATOM | 1813 | CB | MET | A | 760 | −2.032 | −5.063 | 15.874 | 1.00 | 18.91 |
| ATOM | 1814 | CG | MET | A | 760 | −1.140 | −4.422 | 14.835 | 1.00 | 19.09 |
| ATOM | 1815 | SD | MET | A | 760 | −0.717 | −2.696 | 15.216 | 1.00 | 18.56 |
| ATOM | 1816 | CE | MET | A | 760 | −2.370 | −1.939 | 15.227 | 1.00 | 17.91 |
| ATOM | 1817 | C | MET | A | 760 | −2.468 | −5.487 | 18.302 | 1.00 | 18.32 |
| ATOM | 1818 | O | MET | A | 760 | −2.808 | −4.590 | 19.072 | 1.00 | 15.93 |
| ATOM | 1819 | N | THR | A | 761 | −3.002 | −6.706 | 18.350 | 1.00 | 17.94 |
| ATOM | 1820 | CA | THR | A | 761 | −4.024 | −7.021 | 19.336 | 1.00 | 17.15 |
| ATOM | 1821 | CB | THR | A | 761 | −4.540 | −8.454 | 19.164 | 1.00 | 18.32 |
| ATOM | 1822 | OG1 | THR | A | 761 | −5.103 | −8.594 | 17.856 | 1.00 | 18.56 |
| ATOM | 1823 | CG2 | THR | A | 761 | −5.594 | −8.772 | 20.220 | 1.00 | 17.55 |
| ATOM | 1824 | C | THR | A | 761 | −3.446 | −6.880 | 20.746 | 1.00 | 17.99 |
| ATOM | 1825 | O | THR | A | 761 | −4.089 | −6.332 | 21.635 | 1.00 | 19.05 |
| ATOM | 1826 | N | ALA | A | 762 | −2.228 | −7.381 | 20.946 | 1.00 | 16.64 |
| ATOM | 1827 | CA | ALA | A | 762 | −1.590 | −7.298 | 22.255 | 1.00 | 16.82 |
| ATOM | 1828 | CB | ALA | A | 762 | −0.194 | −7.908 | 22.202 | 1.00 | 19.67 |
| ATOM | 1829 | C | ALA | A | 762 | −1.514 | −5.854 | 22.755 | 1.00 | 18.11 |
| ATOM | 1830 | O | ALA | A | 762 | −1.766 | −5.585 | 23.933 | 1.00 | 16.84 |
| ATOM | 1831 | N | CYS | A | 763 | −1.169 | −4.936 | 21.860 | 1.00 | 15.07 |
| ATOM | 1832 | CA | CYS | A | 763 | −1.081 | −3.523 | 22.216 | 1.00 | 16.86 |
| ATOM | 1833 | CB | CYS | A | 763 | −0.303 | −2.767 | 21.147 | 1.00 | 16.42 |
| ATOM | 1834 | SG | CYS | A | 763 | 1.450 | −3.220 | 21.036 | 1.00 | 16.68 |
| ATOM | 1835 | C | CYS | A | 763 | −2.469 | −2.904 | 22.372 | 1.00 | 18.36 |
| ATOM | 1836 | O | CYS | A | 763 | −2.705 | −2.091 | 23.269 | 1.00 | 15.92 |
| ATOM | 1837 | N | ASP | A | 764 | −3.385 | −3.289 | 21.494 | 1.00 | 17.47 |
| ATOM | 1838 | CA | ASP | A | 764 | −4.743 | −2.766 | 21.528 | 1.00 | 18.98 |
| ATOM | 1839 | CB | ASP | A | 764 | −5.539 | −3.360 | 20.360 | 1.00 | 18.28 |
| ATOM | 1840 | CG | ASP | A | 764 | −6.834 | −2.624 | 20.089 | 1.00 | 19.27 |
| ATOM | 1841 | OD1 | ASP | A | 764 | −7.670 | −3.161 | 19.333 | 1.00 | 22.03 |
| ATOM | 1842 | OD2 | ASP | A | 764 | −7.013 | −1.507 | 20.614 | 1.00 | 18.63 |
| ATOM | 1843 | C | ASP | A | 764 | −5.446 | −3.078 | 22.857 | 1.00 | 18.67 |
| ATOM | 1844 | O | ASP | A | 764 | −6.269 | −2.290 | 23.331 | 1.00 | 19.51 |
| ATOM | 1845 | N | LEU | A | 765 | −5.126 | −4.217 | 23.470 | 1.00 | 16.07 |
| ATOM | 1846 | CA | LEU | A | 765 | −5.771 | −4.596 | 24.726 | 1.00 | 16.91 |
| ATOM | 1847 | CB | LEU | A | 765 | −6.108 | −6.098 | 24.712 | 1.00 | 22.31 |
| ATOM | 1848 | CG | LEU | A | 765 | −6.925 | −6.653 | 23.540 | 1.00 | 22.32 |
| ATOM | 1849 | CD1 | LEU | A | 765 | −7.143 | −8.152 | 23.733 | 1.00 | 22.41 |
| ATOM | 1850 | CD2 | LEU | A | 765 | −8.260 | −5.940 | 23.451 | 1.00 | 22.60 |
| ATOM | 1851 | C | LEU | A | 765 | −4.911 | −4.288 | 25.953 | 1.00 | 17.63 |
| ATOM | 1852 | O | LEU | A | 765 | −5.250 | −4.699 | 27.062 | 1.00 | 18.64 |
| ATOM | 1853 | N | SER | A | 766 | −3.830 | −3.541 | 25.750 | 1.00 | 17.02 |
| ATOM | 1854 | CA | SER | A | 766 | −2.869 | −3.230 | 26.812 | 1.00 | 18.24 |
| ATOM | 1855 | CB | SER | A | 766 | −1.846 | −2.205 | 26.312 | 1.00 | 20.09 |
| ATOM | 1856 | OG | SER | A | 766 | −2.378 | −0.892 | 26.319 | 1.00 | 21.90 |
| ATOM | 1857 | C | SER | A | 766 | −3.400 | −2.775 | 28.171 | 1.00 | 17.35 |
| ATOM | 1858 | O | SER | A | 766 | −2.738 | −2.975 | 29.197 | 1.00 | 16.73 |
| ATOM | 1859 | N | ALA | A | 767 | −4.571 | −2.152 | 28.196 | 1.00 | 18.41 |
| ATOM | 1860 | CA | ALA | A | 767 | −5.113 | −1.682 | 29.466 | 1.00 | 19.58 |
| ATOM | 1861 | CB | ALA | A | 767 | −6.465 | −0.994 | 29.242 | 1.00 | 14.05 |
| ATOM | 1862 | C | ALA | A | 767 | −5.272 | −2.823 | 30.461 | 1.00 | 20.35 |
| ATOM | 1863 | O | ALA | A | 767 | −5.111 | −2.636 | 31.666 | 1.00 | 20.93 |
| ATOM | 1864 | N | ILE | A | 768 | −5.580 | −4.008 | 29.949 | 1.00 | 18.87 |
| ATOM | 1865 | CA | ILE | A | 768 | −5.789 | −5.176 | 30.786 | 1.00 | 20.62 |
| ATOM | 1866 | CB | ILE | A | 768 | −6.405 | −6.330 | 29.943 | 1.00 | 19.42 |
| ATOM | 1867 | CG2 | ILE | A | 768 | −5.323 | −7.008 | 29.112 | 1.00 | 20.41 |
| ATOM | 1868 | CG1 | ILE | A | 768 | −7.087 | −7.351 | 30.856 | 1.00 | 20.44 |
| ATOM | 1869 | CD1 | ILE | A | 768 | −8.333 | −6.831 | 31.548 | 1.00 | 20.90 |
| ATOM | 1870 | C | ILE | A | 768 | −4.499 | −5.657 | 31.466 | 1.00 | 20.39 |
| ATOM | 1871 | O | ILE | A | 768 | −4.543 | −6.544 | 32.307 | 1.00 | 21.21 |
| ATOM | 1872 | N | THR | A | 769 | −3.364 | −5.061 | 31.108 | 1.00 | 19.18 |
| ATOM | 1873 | CA | THR | A | 769 | −2.075 | −5.437 | 31.693 | 1.00 | 19.16 |
| ATOM | 1874 | CB | THR | A | 769 | −0.996 | −5.672 | 30.620 | 1.00 | 20.58 |
| ATOM | 1875 | OG1 | THR | A | 769 | −0.519 | −4.408 | 30.131 | 1.00 | 19.24 |
| ATOM | 1876 | CG2 | THR | A | 769 | −1.561 | −6.478 | 29.453 | 1.00 | 20.09 |
| ATOM | 1877 | C | THR | A | 769 | −1.496 | −4.379 | 32.637 | 1.00 | 21.13 |
| ATOM | 1878 | O | THR | A | 769 | −0.488 | −4.618 | 33.300 | 1.00 | 21.47 |
| ATOM | 1879 | N | LYS | A | 770 | −2.127 | −3.214 | 32.691 | 1.00 | 19.99 |
| ATOM | 1880 | CA | LYS | A | 770 | −1.634 | −2.120 | 33.527 | 1.00 | 20.68 |
| ATOM | 1881 | CB | LYS | A | 770 | −2.313 | −0.812 | 33.108 | 1.00 | 20.27 |
| ATOM | 1882 | CG | LYS | A | 770 | −2.103 | −0.409 | 31.660 | 1.00 | 19.98 |
| ATOM | 1883 | CD | LYS | A | 770 | −0.711 | 0.136 | 31.431 | 1.00 | 20.21 |
| ATOM | 1884 | CE | LYS | A | 770 | −0.502 | 0.464 | 29.960 | 1.00 | 20.34 |
| ATOM | 1885 | NZ | LYS | A | 770 | 0.829 | 1.065 | 29.701 | 1.00 | 21.28 |
| ATOM | 1886 | C | LYS | A | 770 | −1.861 | −2.330 | 35.026 | 1.00 | 19.97 |
| ATOM | 1887 | O | LYS | A | 770 | −2.628 | −3.204 | 35.437 | 1.00 | 20.86 |
| ATOM | 1888 | N | PRO | A | 771 | −1.186 | −1.521 | 35.866 | 1.00 | 23.24 |
| ATOM | 1889 | CD | PRO | A | 771 | −0.150 | −0.537 | 35.512 | 1.00 | 25.67 |
| ATOM | 1890 | CA | PRO | A | 771 | −1.329 | −1.615 | 37.321 | 1.00 | 24.06 |
| ATOM | 1891 | CB | PRO | A | 771 | −0.461 | −0.463 | 37.823 | 1.00 | 25.01 |
| ATOM | 1892 | CG | PRO | A | 771 | 0.636 | −0.421 | 36.809 | 1.00 | 24.38 |
| ATOM | 1893 | C | PRO | A | 771 | −2.804 | −1.429 | 37.672 | 1.00 | 23.94 |
| ATOM | 1894 | O | PRO | A | 771 | −3.529 | −0.723 | 36.970 | 1.00 | 23.81 |
| ATOM | 1895 | N | TRP | A | 772 | −3.236 | −2.057 | 38.759 | 1.00 | 22.70 |
| ATOM | 1896 | CA | TRP | A | 772 | −4.626 | −2.009 | 39.201 | 1.00 | 23.47 |
| ATOM | 1897 | CB | TRP | A | 772 | −4.721 | −2.480 | 40.652 | 1.00 | 31.03 |
| ATOM | 1898 | CG | TRP | A | 772 | −6.120 | −2.631 | 41.148 | 1.00 | 31.98 |
| ATOM | 1899 | CD2 | TRP | A | 772 | −7.205 | −3.295 | 40.484 | 1.00 | 31.41 |
| ATOM | 1900 | CE2 | TRP | A | 772 | −8.322 | −3.228 | 41.345 | 1.00 | 32.26 |
| ATOM | 1901 | CE3 | TRP | A | 772 | −7.342 | −3.940 | 39.246 | 1.00 | 31.94 |
| ATOM | 1902 | CD1 | TRP | A | 772 | −6.611 | −2.199 | 42.346 | 1.00 | 31.60 |
| ATOM | 1903 | NE1 | TRP | A | 772 | −7.930 | −2.553 | 42.471 | 1.00 | 31.93 |
| ATOM | 1904 | CZ2 | TRP | A | 772 | −9.565 | −3.785 | 41.009 | 1.00 | 32.15 |
| ATOM | 1905 | CZ3 | TRP | A | 772 | −8.579 | −4.493 | 38.912 | 1.00 | 31.45 |
| ATOM | 1906 | CH2 | TRP | A | 772 | −9.672 | −4.411 | 39.792 | 1.00 | 31.98 |
| ATOM | 1907 | C | TRP | A | 772 | −5.368 | −0.678 | 39.057 | 1.00 | 23.85 |
| ATOM | 1908 | O | TRP | A | 772 | −6.409 | −0.609 | 38.399 | 1.00 | 22.22 |
| ATOM | 1909 | N | PRO | A | 773 | −4.857 | 0.398 | 39.678 | 1.00 | 25.77 |
| ATOM | 1910 | CD | PRO | A | 773 | −3.583 | 0.541 | 40.403 | 1.00 | 32.22 |
| ATOM | 1911 | CA | PRO | A | 773 | −5.546 | 1.689 | 39.567 | 1.00 | 26.04 |
| ATOM | 1912 | CB | PRO | A | 773 | −4.609 | 2.650 | 40.304 | 1.00 | 32.61 |
| ATOM | 1913 | CG | PRO | A | 773 | −3.259 | 1.991 | 40.171 | 1.00 | 33.14 |
| ATOM | 1914 | C | PRO | A | 773 | −5.831 | 2.122 | 38.134 | 1.00 | 25.71 |
| ATOM | 1915 | O | PRO | A | 773 | −6.900 | 2.653 | 37.834 | 1.00 | 26.03 |
| ATOM | 1916 | N | ILE | A | 774 | −4.869 | 1.885 | 37.253 | 1.00 | 21.46 |
| ATOM | 1917 | CA | ILE | A | 774 | −5.008 | 2.236 | 35.850 | 1.00 | 20.45 |
| ATOM | 1918 | CB | ILE | A | 774 | −3.648 | 2.105 | 35.135 | 1.00 | 25.41 |
| ATOM | 1919 | CG2 | ILE | A | 774 | −3.790 | 2.436 | 33.654 | 1.00 | 25.27 |
| ATOM | 1920 | CG1 | ILE | A | 774 | −2.633 | 3.037 | 35.802 | 1.00 | 26.17 |
| ATOM | 1921 | CD1 | ILE | A | 774 | −1.205 | 2.807 | 35.355 | 1.00 | 25.47 |
| ATOM | 1922 | C | ILE | A | 774 | −6.038 | 1.329 | 35.173 | 1.00 | 19.51 |
| ATOM | 1923 | O | ILE | A | 774 | −6.928 | 1.804 | 34.471 | 1.00 | 18.75 |
| ATOM | 1924 | N | GLN | A | 775 | −5.925 | 0.021 | 35.388 | 1.00 | 22.02 |
| ATOM | 1925 | CA | GLN | A | 775 | −6.868 | −0.916 | 34.783 | 1.00 | 21.75 |
| ATOM | 1926 | CB | GLN | A | 775 | −6.491 | −2.361 | 35.135 | 1.00 | 20.94 |
| ATOM | 1927 | CG | GLN | A | 775 | −7.668 | −3.340 | 35.192 | 1.00 | 21.03 |
| ATOM | 1928 | CD | GLN | A | 775 | −8.395 | −3.531 | 33.864 | 1.00 | 21.36 |
| ATOM | 1929 | OE1 | GLN | A | 775 | −9.457 | −4.141 | 33.826 | 1.00 | 21.09 |
| ATOM | 1930 | NE2 | GLN | A | 775 | −7.826 | −3.018 | 32.779 | 1.00 | 21.40 |
| ATOM | 1931 | C | GLN | A | 775 | −8.300 | −0.626 | 35.219 | 1.00 | 21.97 |
| ATOM | 1932 | O | GLN | A | 775 | −9.216 | −0.637 | 34.402 | 1.00 | 21.91 |
| ATOM | 1933 | N | GLN | A | 776 | −8.486 | −0.372 | 36.510 | 1.00 | 21.52 |
| ATOM | 1934 | CA | GLN | A | 776 | −9.807 | −0.066 | 37.051 | 1.00 | 23.77 |
| ATOM | 1935 | CB | GLN | A | 776 | −9.709 | 0.263 | 38.537 | 1.00 | 38.83 |
| ATOM | 1936 | CG | GLN | A | 776 | −9.798 | −0.919 | 39.456 | 1.00 | 39.61 |
| ATOM | 1937 | CD | GLN | A | 776 | −9.747 | −0.503 | 40.910 | 1.00 | 39.43 |
| ATOM | 1938 | OE1 | GLN | A | 776 | −8.717 | −0.035 | 41.397 | 1.00 | 39.77 |
| ATOM | 1939 | NE2 | GLN | A | 776 | −10.865 | −0.662 | 41.610 | 1.00 | 39.38 |
| ATOM | 1940 | C | GLN | A | 776 | −10.420 | 1.133 | 36.350 | 1.00 | 23.07 |
| ATOM | 1941 | O | GLN | A | 776 | −11.582 | 1.106 | 35.942 | 1.00 | 24.26 |
| ATOM | 1942 | N | ARG | A | 777 | −9.628 | 2.190 | 36.231 | 1.00 | 23.71 |
| ATOM | 1943 | CA | ARG | A | 777 | −10.081 | 3.413 | 35.598 | 1.00 | 23.28 |
| ATOM | 1944 | CB | ARG | A | 777 | −8.993 | 4.489 | 35.692 | 1.00 | 27.59 |
| ATOM | 1945 | CG | ARG | A | 777 | −9.423 | 5.850 | 35.159 | 1.00 | 27.74 |
| ATOM | 1946 | CD | ARG | A | 777 | −8.305 | 6.873 | 35.280 | 1.00 | 27.86 |
| ATOM | 1947 | NE | ARG | A | 777 | −7.155 | 6.535 | 34.445 | 1.00 | 27.75 |
| ATOM | 1948 | CZ | ARG | A | 777 | −7.161 | 6.545 | 33.114 | 1.00 | 27.57 |
| ATOM | 1949 | NH1 | ARG | A | 777 | −8.257 | 6.881 | 32.448 | 1.00 | 28.23 |
| ATOM | 1950 | NH2 | ARG | A | 777 | −6.066 | 6.216 | 32.446 | 1.00 | 28.20 |
| ATOM | 1951 | C | ARG | A | 777 | −10.465 | 3.191 | 34.139 | 1.00 | 23.20 |
| ATOM | 1952 | O | ARG | A | 777 | −11.537 | 3.605 | 33.704 | 1.00 | 22.51 |
| ATOM | 1953 | N | ILE | A | 778 | −9.592 | 2.535 | 33.380 | 1.00 | 17.78 |
| ATOM | 1954 | CA | ILE | A | 778 | −9.884 | 2.297 | 31.972 | 1.00 | 17.96 |
| ATOM | 1955 | CB | ILE | A | 778 | −8.633 | 1.806 | 31.225 | 1.00 | 19.43 |
| ATOM | 1956 | CG2 | ILE | A | 778 | −8.967 | 1.520 | 29.764 | 1.00 | 18.09 |
| ATOM | 1957 | CG1 | ILE | A | 778 | −7.560 | 2.894 | 31.305 | 1.00 | 19.35 |
| ATOM | 1958 | CD1 | ILE | A | 778 | −6.214 | 2.505 | 30.738 | 1.00 | 19.60 |
| ATOM | 1959 | C | ILE | A | 778 | −11.038 | 1.323 | 31.796 | 1.00 | 18.56 |
| ATOM | 1960 | O | ILE | A | 778 | −11.872 | 1.502 | 30.903 | 1.00 | 18.79 |
| ATOM | 1961 | N | ALA | A | 779 | −11.106 | 0.309 | 32.654 | 1.00 | 20.16 |
| ATOM | 1962 | CA | ALA | A | 779 | −12.199 | −0.656 | 32.590 | 1.00 | 21.21 |
| ATOM | 1963 | CB | ALA | A | 779 | −12.066 | −1.680 | 33.708 | 1.00 | 24.43 |
| ATOM | 1964 | C | ALA | A | 779 | −13.531 | 0.091 | 32.712 | 1.00 | 21.19 |
| ATOM | 1965 | O | ALA | A | 779 | −14.519 | −0.280 | 32.085 | 1.00 | 20.38 |
| ATOM | 1966 | N | GLU | A | 780 | −13.558 | 1.147 | 33.518 | 1.00 | 20.96 |
| ATOM | 1967 | CA | GLU | A | 780 | −14.786 | 1.926 | 33.682 | 1.00 | 22.15 |
| ATOM | 1968 | CB | GLU | A | 780 | −14.652 | 2.894 | 34.863 | 1.00 | 51.37 |
| ATOM | 1969 | CG | GLU | A | 780 | −14.497 | 2.196 | 36.206 | 1.00 | 52.40 |
| ATOM | 1970 | CD | GLU | A | 780 | −14.449 | 3.163 | 37.373 | 1.00 | 53.03 |
| ATOM | 1971 | OE1 | GLU | A | 780 | −13.590 | 4.072 | 37.359 | 1.00 | 53.05 |
| ATOM | 1972 | OE2 | GLU | A | 780 | −15.266 | 3.013 | 38.306 | 1.00 | 52.97 |
| ATOM | 1973 | C | GLU | A | 780 | −15.147 | 2.696 | 32.407 | 1.00 | 21.53 |
| ATOM | 1974 | O | GLU | A | 780 | −16.326 | 2.918 | 32.127 | 1.00 | 20.90 |
| ATOM | 1975 | N | LEU | A | 781 | −14.134 | 3.105 | 31.642 | 1.00 | 18.46 |
| ATOM | 1976 | CA | LEU | A | 781 | −14.365 | 3.818 | 30.390 | 1.00 | 19.53 |
| ATOM | 1977 | CB | LEU | A | 781 | −13.050 | 4.399 | 29.847 | 1.00 | 20.51 |
| ATOM | 1978 | CG | LEU | A | 781 | −12.392 | 5.516 | 30.672 | 1.00 | 21.84 |
| ATOM | 1979 | CD1 | LEU | A | 781 | −11.131 | 6.026 | 29.976 | 1.00 | 21.36 |
| ATOM | 1980 | CD2 | LEU | A | 781 | −13.389 | 6.660 | 30.828 | 1.00 | 20.48 |
| ATOM | 1981 | C | LEU | A | 781 | −14.949 | 2.824 | 29.389 | 1.00 | 19.65 |
| ATOM | 1982 | O | LEU | A | 781 | −15.898 | 3.133 | 28.654 | 1.00 | 19.62 |
| ATOM | 1983 | N | VAL | A | 782 | −14.368 | 1.628 | 29.362 | 1.00 | 21.76 |
| ATOM | 1984 | CA | VAL | A | 782 | −14.837 | 0.569 | 28.474 | 1.00 | 22.54 |
| ATOM | 1985 | CB | VAL | A | 782 | −14.026 | −0.740 | 28.693 | 1.00 | 22.92 |
| ATOM | 1986 | CG1 | VAL | A | 782 | −14.659 | −1.889 | 27.931 | 1.00 | 21.63 |
| ATOM | 1987 | CG2 | VAL | A | 782 | −12.588 | −0.554 | 28.231 | 1.00 | 22.05 |
| ATOM | 1988 | C | VAL | A | 782 | −16.311 | 0.290 | 28.766 | 1.00 | 21.93 |
| ATOM | 1989 | O | VAL | A | 782 | −17.142 | 0.238 | 27.857 | 1.00 | 23.01 |
| ATOM | 1990 | N | ALA | A | 783 | −16.632 | 0.123 | 30.045 | 1.00 | 19.50 |
| ATOM | 1991 | CA | ALA | A | 783 | −18.000 | −0.166 | 30.445 | 1.00 | 21.17 |
| ATOM | 1992 | CB | ALA | A | 783 | −18.061 | −0.447 | 31.942 | 1.00 | 21.14 |
| ATOM | 1993 | C | ALA | A | 783 | −18.940 | 0.979 | 30.078 | 1.00 | 20.67 |
| ATOM | 1994 | O | ALA | A | 783 | −20.051 | 0.755 | 29.598 | 1.00 | 21.82 |
| ATOM | 1995 | N | THR | A | 784 | −18.490 | 2.209 | 30.291 | 1.00 | 23.13 |
| ATOM | 1996 | CA | THR | A | 784 | −19.312 | 3.362 | 29.962 | 1.00 | 23.26 |
| ATOM | 1997 | CB | THR | A | 784 | −18.577 | 4.668 | 30.312 | 1.00 | 24.94 |
| ATOM | 1998 | OG1 | THR | A | 784 | −18.379 | 4.726 | 31.731 | 1.00 | 25.01 |
| ATOM | 1999 | CG2 | THR | A | 784 | −19.379 | 5.877 | 29.864 | 1.00 | 24.89 |
| ATOM | 2000 | C | THR | A | 784 | −19.702 | 3.364 | 28.481 | 1.00 | 24.33 |
| ATOM | 2001 | O | THR | A | 784 | −20.881 | 3.543 | 28.130 | 1.00 | 22.64 |
| ATOM | 2002 | N | GLU | A | 785 | −18.720 | 3.153 | 27.612 | 1.00 | 19.82 |
| ATOM | 2003 | CA | GLU | A | 785 | −18.991 | 3.136 | 26.182 | 1.00 | 21.00 |
| ATOM | 2004 | CB | GLU | A | 785 | −17.678 | 2.998 | 25.387 | 1.00 | 21.06 |
| ATOM | 2005 | CG | GLU | A | 785 | −17.871 | 2.918 | 23.873 | 1.00 | 20.31 |
| ATOM | 2006 | CD | GLU | A | 785 | −16.575 | 3.080 | 23.081 | 1.00 | 21.05 |
| ATOM | 2007 | OE1 | GLU | A | 785 | −16.506 | 2.557 | 21.947 | 1.00 | 20.57 |
| ATOM | 2008 | OE2 | GLU | A | 785 | −15.637 | 3.740 | 23.572 | 1.00 | 18.86 |
| ATOM | 2009 | C | GLU | A | 785 | −19.965 | 2.006 | 25.846 | 1.00 | 20.29 |
| ATOM | 2010 | O | GLU | A | 785 | −20.905 | 2.211 | 25.083 | 1.00 | 20.32 |
| ATOM | 2011 | N | PHE | A | 786 | −19.753 | 0.824 | 26.422 | 1.00 | 21.26 |
| ATOM | 2012 | CA | PHE | A | 786 | −20.643 | −0.307 | 26.161 | 1.00 | 21.97 |
| ATOM | 2013 | CB | PHE | A | 786 | −20.185 | −1.567 | 26.901 | 1.00 | 26.06 |
| ATOM | 2014 | CG | PHE | A | 786 | −18.955 | −2.216 | 26.324 | 1.00 | 27.41 |
| ATOM | 2015 | CD1 | PHE | A | 786 | −18.629 | −2.067 | 24.978 | 1.00 | 27.06 |
| ATOM | 2016 | CD2 | PHE | A | 786 | −18.146 | −3.021 | 27.122 | 1.00 | 26.60 |
| ATOM | 2017 | CE1 | PHE | A | 786 | −17.519 | −2.711 | 24.437 | 1.00 | 27.27 |
| ATOM | 2018 | CE2 | PHE | A | 786 | −17.032 | −3.672 | 26.586 | 1.00 | 27.24 |
| ATOM | 2019 | CZ | PHE | A | 786 | −16.722 | −3.515 | 25.244 | 1.00 | 27.18 |
| ATOM | 2020 | C | PHE | A | 786 | −22.068 | 0.011 | 26.599 | 1.00 | 23.40 |
| ATOM | 2021 | O | PHE | A | 786 | −23.023 | −0.269 | 25.878 | 1.00 | 21.69 |
| ATOM | 2022 | N | PHE | A | 787 | −22.206 | 0.594 | 27.787 | 1.00 | 23.44 |
| ATOM | 2023 | CA | PHE | A | 787 | −23.530 | 0.926 | 28.297 | 1.00 | 22.70 |
| ATOM | 2024 | CB | PHE | A | 787 | −23.445 | 1.413 | 29.751 | 1.00 | 22.93 |
| ATOM | 2025 | CG | PHE | A | 787 | −22.945 | 0.370 | 30.719 | 1.00 | 21.91 |
| ATOM | 2026 | CD1 | PHE | A | 787 | −23.122 | −0.991 | 30.464 | 1.00 | 22.94 |
| ATOM | 2027 | CD2 | PHE | A | 787 | −22.335 | 0.749 | 31.910 | 1.00 | 23.07 |
| ATOM | 2028 | CE1 | PHE | A | 787 | −22.697 | −1.954 | 31.383 | 1.00 | 23.17 |
| ATOM | 2029 | CE2 | PHE | A | 787 | −21.907 | −0.201 | 32.833 | 1.00 | 22.50 |
| ATOM | 2030 | CZ | PHE | A | 787 | −22.087 | −1.555 | 32.572 | 1.00 | 23.33 |
| ATOM | 2031 | C | PHE | A | 787 | −24.215 | 1.968 | 27.417 | 1.00 | 23.79 |
| ATOM | 2032 | O | PHE | A | 787 | −25.431 | 1.906 | 27.205 | 1.00 | 21.92 |
| ATOM | 2033 | N | ASP | A | 788 | −23.442 | 2.912 | 26.884 | 1.00 | 23.70 |
| ATOM | 2034 | CA | ASP | A | 788 | −24.021 | 3.923 | 26.010 | 1.00 | 25.33 |
| ATOM | 2035 | CB | ASP | A | 788 | −22.978 | 4.959 | 25.591 | 1.00 | 54.55 |
| ATOM | 2036 | CG | ASP | A | 788 | −23.605 | 6.296 | 25.233 | 1.00 | 55.82 |
| ATOM | 2037 | OD1 | ASP | A | 788 | −24.157 | 6.955 | 26.141 | 1.00 | 56.10 |
| ATOM | 2038 | OD2 | ASP | A | 788 | −23.558 | 6.686 | 24.046 | 1.00 | 55.97 |
| ATOM | 2039 | C | ASP | A | 788 | −24.571 | 3.216 | 24.774 | 1.00 | 24.36 |
| ATOM | 2040 | O | ASP | A | 788 | −25.646 | 3.551 | 24.283 | 1.00 | 25.06 |
| ATOM | 2041 | N | GLN | A | 789 | −23.837 | 2.235 | 24.257 | 1.00 | 20.19 |
| ATOM | 2042 | CA | GLN | A | 789 | −24.350 | 1.522 | 23.101 | 1.00 | 19.43 |
| ATOM | 2043 | CB | GLN | A | 789 | −23.340 | 0.508 | 22.566 | 1.00 | 19.05 |
| ATOM | 2044 | CG | GLN | A | 789 | −23.987 | −0.433 | 21.563 | 1.00 | 19.55 |
| ATOM | 2045 | CD | GLN | A | 789 | −23.011 | −1.268 | 20.781 | 1.00 | 19.48 |
| ATOM | 2046 | OE1 | GLN | A | 789 | −23.388 | −2.299 | 20.222 | 1.00 | 20.82 |
| ATOM | 2047 | NE2 | GLN | A | 789 | −21.755 | −0.833 | 20.718 | 1.00 | 18.14 |
| ATOM | 2048 | C | GLN | A | 789 | −25.629 | 0.802 | 23.503 | 1.00 | 20.46 |
| ATOM | 2049 | O | GLN | A | 789 | −26.586 | 0.735 | 22.729 | 1.00 | 20.11 |
| ATOM | 2050 | N | GLY | A | 790 | −25.639 | 0.266 | 24.721 | 1.00 | 22.31 |
| ATOM | 2051 | CA | GLY | A | 790 | −26.810 | −0.436 | 25.216 | 1.00 | 24.09 |
| ATOM | 2052 | C | GLY | A | 790 | −28.031 | 0.463 | 25.175 | 1.00 | 25.10 |
| ATOM | 2053 | O | GLY | A | 790 | −29.115 | 0.025 | 24.783 | 1.00 | 24.31 |
| ATOM | 2054 | N | ASP | A | 791 | −27.857 | 1.722 | 25.573 | 1.00 | 25.00 |
| ATOM | 2055 | CA | ASP | A | 791 | −28.963 | 2.683 | 25.568 | 1.00 | 25.93 |
| ATOM | 2056 | CB | ASP | A | 791 | −28.525 | 4.026 | 26.160 | 1.00 | 29.26 |
| ATOM | 2057 | CG | ASP | A | 791 | −28.107 | 3.918 | 27.611 | 1.00 | 29.25 |
| ATOM | 2058 | OD1 | ASP | A | 791 | −28.575 | 2.987 | 28.297 | 1.00 | 29.11 |
| ATOM | 2059 | OD2 | ASP | A | 791 | −27.323 | 4.778 | 28.068 | 1.00 | 30.04 |
| ATOM | 2060 | C | ASP | A | 791 | −29.464 | 2.909 | 24.143 | 1.00 | 25.33 |
| ATOM | 2061 | O | ASP | A | 791 | −30.669 | 3.029 | 23.907 | 1.00 | 26.34 |
| ATOM | 2062 | N | ARG | A | 792 | −28.527 | 2.984 | 23.205 | 1.00 | 25.90 |
| ATOM | 2063 | CA | ARG | A | 792 | −28.842 | 3.185 | 21.794 | 1.00 | 25.99 |
| ATOM | 2064 | CB | ARG | A | 792 | −27.546 | 3.326 | 20.990 | 1.00 | 34.02 |
| ATOM | 2065 | CG | ARG | A | 792 | −26.801 | 4.633 | 21.228 | 1.00 | 34.62 |
| ATOM | 2066 | CD | ARG | A | 792 | −27.310 | 5.716 | 20.300 | 1.00 | 35.57 |
| ATOM | 2067 | NE | ARG | A | 792 | −26.820 | 5.533 | 18.935 | 1.00 | 35.28 |
| ATOM | 2068 | CZ | ARG | A | 792 | −25.591 | 5.845 | 18.537 | 1.00 | 34.46 |
| ATOM | 2069 | NH1 | ARG | A | 792 | −24.726 | 6.358 | 19.403 | 1.00 | 34.52 |
| ATOM | 2070 | NH2 | ARG | A | 792 | −25.225 | 5.647 | 17.278 | 1.00 | 34.52 |
| ATOM | 2071 | C | ARG | A | 792 | −29.645 | 2.005 | 21.261 | 1.00 | 26.14 |
| ATOM | 2072 | O | ARG | A | 792 | −30.634 | 2.179 | 20.553 | 1.00 | 26.08 |
| ATOM | 2073 | N | GLU | A | 793 | −29.219 | 0.797 | 21.608 | 1.00 | 25.23 |
| ATOM | 2074 | CA | GLU | A | 793 | −29.910 | −0.396 | 21.141 | 1.00 | 25.15 |
| ATOM | 2075 | CB | GLU | A | 793 | −29.156 | −1.650 | 21.584 | 1.00 | 28.67 |
| ATOM | 2076 | CG | GLU | A | 793 | −27.715 | −1.688 | 21.120 | 1.00 | 29.21 |
| ATOM | 2077 | CD | GLU | A | 793 | −27.005 | −2.958 | 21.543 | 1.00 | 29.03 |
| ATOM | 2078 | OE1 | GLU | A | 793 | −27.219 | −3.406 | 22.689 | 1.00 | 28.67 |
| ATOM | 2079 | OE2 | GLU | A | 793 | −26.222 | −3.501 | 20.737 | 1.00 | 28.60 |
| ATOM | 2080 | C | GLU | A | 793 | −31.343 | −0.444 | 21.660 | 1.00 | 26.11 |
| ATOM | 2081 | O | GLU | A | 793 | −32.254 | −0.858 | 20.945 | 1.00 | 25.51 |
| ATOM | 2082 | N | ARG | A | 794 | −31.546 | −0.022 | 22.904 | 1.00 | 30.47 |
| ATOM | 2083 | CA | ARG | A | 794 | −32.887 | −0.041 | 23.477 | 1.00 | 30.88 |
| ATOM | 2084 | CB | ARG | A | 794 | −32.861 | 0.402 | 24.945 | 1.00 | 42.43 |
| ATOM | 2085 | CG | ARO | A | 794 | −31.776 | −0.241 | 25.786 | 1.00 | 42.99 |
| ATOM | 2086 | CD | ARG | A | 794 | −31.978 | 0.056 | 27.266 | 1.00 | 43.11 |
| ATOM | 2087 | NE | ARG | A | 794 | −32.839 | −0.939 | 27.893 | 1.00 | 43.32 |
| ATOM | 2088 | CZ | ARG | A | 794 | −32.425 | −1.820 | 28.798 | 1.00 | 42.83 |
| ATOM | 2089 | NH1 | ARG | A | 794 | −31.158 | −1.826 | 29.194 | 1.00 | 43.06 |
| ATOM | 2090 | NH2 | ARG | A | 794 | −33.274 | −2.708 | 29.295 | 1.00 | 43.25 |
| ATOM | 2091 | C | ARG | A | 794 | −33.818 | 0.886 | 22.701 | 1.00 | 31.12 |
| ATOM | 2092 | O | ARG | A | 794 | −34.972 | 0.547 | 22.451 | 1.00 | 30.32 |
| ATOM | 2093 | N | LYS | A | 795 | −33.307 | 2.050 | 22.314 | 1.00 | 28.47 |
| ATOM | 2094 | CA | LYS | A | 795 | −34.111 | 3.036 | 21.597 | 1.00 | 30.19 |
| ATOM | 2095 | CB | LYS | A | 795 | −33.538 | 4.438 | 21.833 | 1.00 | 43.10 |
| ATOM | 2096 | CG | LYS | A | 795 | −33.084 | 4.688 | 23.262 | 1.00 | 43.51 |
| ATOM | 2097 | CD | LYS | A | 795 | −32.642 | 6.129 | 23.482 | 1.00 | 44.03 |
| ATOM | 2098 | CE | LYS | A | 795 | −33.794 | 6.991 | 23.976 | 1.00 | 43.69 |
| ATOM | 2099 | NZ | LYS | A | 795 | −34.961 | 6.964 | 23.051 | 1.00 | 43.61 |
| ATOM | 2100 | C | LYS | A | 795 | −34.238 | 2.799 | 20.093 | 1.00 | 29.44 |
| ATOM | 2101 | O | LYS | A | 795 | −35.341 | 2.832 | 19.545 | 1.00 | 29.39 |
| ATOM | 2102 | N | GLU | A | 796 | −33.110 | 2.558 | 19.433 | 1.00 | 32.97 |
| ATOM | 2103 | CA | GLU | A | 796 | −33.080 | 2.358 | 17.987 | 1.00 | 33.23 |
| ATOM | 2104 | CB | GLU | A | 796 | −31.723 | 2.803 | 17.443 | 1.00 | 31.41 |
| ATOM | 2105 | CG | GLU | A | 796 | −31.265 | 4.158 | 17.957 | 1.00 | 31.18 |
| ATOM | 2106 | CD | GLU | A | 796 | −29.913 | 4.573 | 17.401 | 1.00 | 31.42 |
| ATOM | 2107 | OE1 | GLU | A | 796 | −29.334 | 5.558 | 17.904 | 1.00 | 31.78 |
| ATOM | 2108 | OE2 | GLU | A | 796 | −29.432 | 3.917 | 16.455 | 1.00 | 32.26 |
| ATOM | 2109 | C | GLU | A | 796 | −33.376 | 0.939 | 17.496 | 1.00 | 33.01 |
| ATOM | 2110 | O | GLU | A | 796 | −33.778 | 0.750 | 16.347 | 1.00 | 33.78 |
| ATOM | 2111 | N | LEU | A | 797 | −33.170 | −0.058 | 18.347 | 1.00 | 29.85 |
| ATOM | 2112 | CA | LEU | A | 797 | −33.424 | −1.438 | 17.952 | 1.00 | 29.25 |
| ATOM | 2113 | CB | LEU | A | 797 | −32.134 | −2.261 | 18.059 | 1.00 | 32.07 |
| ATOM | 2114 | CG | LEU | A | 797 | −30.951 | −1.828 | 17.185 | 1.00 | 32.45 |
| ATOM | 2115 | CD1 | LEU | A | 797 | −29.759 | −2.731 | 17.475 | 1.00 | 32.42 |
| ATOM | 2116 | CD2 | LEU | A | 797 | −31.325 | −1.904 | 15.709 | 1.00 | 32.29 |
| ATOM | 2117 | C | LEU | A | 797 | −34.513 | −2.073 | 18.807 | 1.00 | 29.52 |
| ATOM | 2118 | O | LEU | A | 797 | −34.954 | −3.192 | 18.534 | 1.00 | 29.88 |
| ATOM | 2119 | N | ASN | A | 798 | −34.940 | −1.348 | 19.839 | 1.00 | 31.24 |
| ATOM | 2120 | CA | ASN | A | 798 | −35.972 | −1.815 | 20.757 | 1.00 | 31.79 |
| ATOM | 2121 | CB | ASN | A | 798 | −37.332 | −1.805 | 20.062 | 1.00 | 46.67 |
| ATOM | 2122 | CG | ASN | A | 798 | −37.975 | −0.439 | 20.083 | 1.00 | 47.32 |
| ATOM | 2123 | OD1 | ASN | A | 798 | −38.405 | 0.038 | 21.136 | 1.00 | 47.67 |
| ATOM | 2124 | ND2 | ASN | A | 798 | −38.037 | 0.208 | 18.924 | 1.00 | 47.41 |
| ATOM | 2125 | C | ASN | A | 798 | −35.694 | −3.189 | 21.338 | 1.00 | 31.17 |
| ATOM | 2126 | O | ASN | A | 798 | −36.593 | −4.016 | 21.468 | 1.00 | 30.69 |
| ATOM | 2127 | N | ILE | A | 799 | −34.438 | −3.427 | 21.694 | 1.00 | 29.19 |
| ATOM | 2128 | CA | ILE | A | 799 | −34.046 | −4.701 | 22.274 | 1.00 | 29.70 |
| ATOM | 2129 | CB | ILE | A | 799 | −33.026 | −5.444 | 21.379 | 1.00 | 28.41 |
| ATOM | 2130 | CG2 | ILE | A | 799 | −33.660 | −5.795 | 20.036 | 1.00 | 29.14 |
| ATOM | 2131 | CG1 | ILE | A | 799 | −31.781 | −4.571 | 21.191 | 1.00 | 28.71 |
| ATOM | 2132 | CD1 | ILE | A | 799 | −30.632 | −5.251 | 20.494 | 1.00 | 28.52 |
| ATOM | 2133 | C | ILE | A | 799 | −33.389 | −4.463 | 23.627 | 1.00 | 29.70 |
| ATOM | 2134 | O | ILE | A | 799 | −32.889 | −3.370 | 23.904 | 1.00 | 29.11 |
| ATOM | 2135 | N | GLU | A | 800 | −33.410 | −5.481 | 24.477 | 1.00 | 31.12 |
| ATOM | 2136 | CA | GLU | A | 800 | −32.764 | −5.369 | 25.775 | 1.00 | 32.32 |
| ATOM | 2137 | CB | GLU | A | 800 | −33.322 | −6.394 | 26.768 | 1.00 | 47.83 |
| ATOM | 2138 | CG | GLU | A | 800 | −32.572 | −6.418 | 28.099 | 1.00 | 48.49 |
| ATOM | 2139 | CD | GLU | A | 800 | −32.999 | −7.559 | 29.007 | 1.00 | 48.67 |
| ATOM | 2140 | OE1 | GLU | A | 800 | −32.393 | −7.713 | 30.091 | 1.00 | 48.86 |
| ATOM | 2141 | OE2 | GLU | A | 800 | −33.938 | −8.299 | 28.640 | 1.00 | 48.41 |
| ATOM | 2142 | C | GLU | A | 800 | −31.309 | −5.692 | 25.474 | 1.00 | 31.85 |
| ATOM | 2143 | O | GLU | A | 800 | −31.009 | −6.731 | 24.888 | 1.00 | 31.72 |
| ATOM | 2144 | N | PRO | A | 801 | −30.386 | −4.801 | 25.855 | 1.00 | 32.29 |
| ATOM | 2145 | CD | PRO | A | 801 | −30.534 | −3.557 | 26.629 | 1.00 | 32.84 |
| ATOM | 2146 | CA | PRO | A | 801 | −28.976 | −5.083 | 25.578 | 1.00 | 32.39 |
| ATOM | 2147 | CB | PRO | A | 801 | −28.281 | −3.798 | 26.019 | 1.00 | 33.48 |
| ATOM | 2148 | CG | PRO | A | 801 | −29.143 | −3.351 | 27.168 | 1.00 | 33.84 |
| ATOM | 2149 | C | PRO | A | 801 | −28.462 | −6.308 | 26.323 | 1.00 | 31.89 |
| ATOM | 2150 | O | PRO | A | 801 | −29.056 | −6.752 | 27.312 | 1.00 | 31.79 |
| ATOM | 2151 | N | THR | A | 802 | −27.362 | −6.863 | 25.826 | 1.00 | 28.76 |
| ATOM | 2152 | CA | THR | A | 802 | −26.739 | −8.016 | 26.456 | 1.00 | 28.15 |
| ATOM | 2153 | CB | THR | A | 802 | −25.626 | −8.603 | 25.569 | 1.00 | 44.26 |
| ATOM | 2154 | OG1 | THR | A | 802 | −25.026 | −9.721 | 26.233 | 1.00 | 45.50 |
| ATOM | 2155 | CG2 | THR | A | 802 | −24.561 | −7.555 | 25.282 | 1.00 | 44.69 |
| ATOM | 2156 | C | THR | A | 802 | −26.139 | −7.521 | 27.767 | 1.00 | 28.29 |
| ATOM | 2157 | O | THR | A | 802 | −25.956 | −6.317 | 27.960 | 1.00 | 27.03 |
| ATOM | 2158 | N | ASP | A | 803 | −25.843 | −8.448 | 28.669 | 1.00 | 28.44 |
| ATOM | 2159 | CA | ASP | A | 803 | −25.275 | −8.094 | 29.963 | 1.00 | 28.64 |
| ATOM | 2160 | CB | ASP | A | 803 | −24.779 | −9.358 | 30.667 | 1.00 | 34.48 |
| ATOM | 2161 | CG | ASP | A | 803 | −25.893 | −10.098 | 31.379 | 1.00 | 34.81 |
| ATOM | 2162 | OD1 | ASP | A | 803 | −27.045 | −10.054 | 30.893 | 1.00 | 34.90 |
| ATOM | 2163 | OD2 | ASP | A | 803 | −25.611 | −10.730 | 32.420 | 1.00 | 35.01 |
| ATOM | 2164 | C | ASP | A | 803 | −24.153 | −7.063 | 29.906 | 1.00 | 28.44 |
| ATOM | 2165 | O | ASP | A | 803 | −24.176 | −6.074 | 30.635 | 1.00 | 28.48 |
| ATOM | 2166 | N | LEU | A | 804 | −23.177 | −7.275 | 29.031 | 1.00 | 25.97 |
| ATOM | 2167 | CA | LEU | A | 804 | −22.058 | −6.348 | 28.952 | 1.00 | 26.25 |
| ATOM | 2168 | CB | LEU | A | 804 | −20.951 | −6.937 | 28.075 | 1.00 | 47.64 |
| ATOM | 2169 | CG | LEU | A | 804 | −21.304 | −7.412 | 26.671 | 1.00 | 48.05 |
| ATOM | 2170 | CD1 | LEU | A | 804 | −21.264 | −6.244 | 25.708 | 1.00 | 48.53 |
| ATOM | 2171 | CD2 | LEU | A | 804 | −20.301 | −8.466 | 26.242 | 1.00 | 48.25 |
| ATOM | 2172 | C | LEU | A | 804 | −22.415 | −4.938 | 28.501 | 1.00 | 26.00 |
| ATOM | 2173 | O | LEU | A | 804 | −21.654 | −4.007 | 28.728 | 1.00 | 25.68 |
| ATOM | 2174 | N | MET | A | 805 | −23.576 | −4.763 | 27.881 | 1.00 | 25.47 |
| ATOM | 2175 | CA | MET | A | 805 | −23.971 | −3.427 | 27.445 | 1.00 | 25.62 |
| ATOM | 2176 | CB | MET | A | 805 | −24.246 | −3.422 | 25.940 | 1.00 | 32.20 |
| ATOM | 2177 | CG | MET | A | 805 | −22.992 | −3.634 | 25.113 | 1.00 | 31.73 |
| ATOM | 2178 | SD | MET | A | 805 | −23.263 | −3.668 | 23.345 | 1.00 | 32.78 |
| ATOM | 2179 | CE | MET | A | 805 | −21.557 | −3.485 | 22.751 | 1.00 | 31.70 |
| ATOM | 2180 | C | MET | A | 805 | −25.186 | −2.920 | 28.209 | 1.00 | 24.74 |
| ATOM | 2181 | O | MET | A | 805 | −25.808 | −1.932 | 27.813 | 1.00 | 25.55 |
| ATOM | 2182 | N | ASN | A | 806 | −25.502 | −3.596 | 29.311 | 1.00 | 23.96 |
| ATOM | 2183 | CA | ASN | A | 806 | −26.645 | −3.239 | 30.151 | 1.00 | 25.57 |
| ATOM | 2184 | CB | ASN | A | 806 | −27.467 | −4.495 | 30.463 | 1.00 | 28.17 |
| ATOM | 2185 | CG | ASN | A | 806 | −28.738 | −4.190 | 31.241 | 1.00 | 28.82 |
| ATOM | 2186 | OD1 | ASN | A | 806 | −28.985 | −3.049 | 31.628 | 1.00 | 28.36 |
| ATOM | 2187 | ND2 | ASN | A | 806 | −29.550 | −5.216 | 31.474 | 1.00 | 28.76 |
| ATOM | 2188 | C | ASN | A | 806 | −26.159 | −2.592 | 31.448 | 1.00 | 25.52 |
| ATOM | 2189 | O | ASN | A | 806 | −25.648 | −3.273 | 32.338 | 1.00 | 25.16 |
| ATOM | 2190 | N | ARG | A | 807 | −26.321 | −1.276 | 31.544 | 1.00 | 25.21 |
| ATOM | 2191 | CA | ARG | A | 807 | −25.892 | −0.512 | 32.717 | 1.00 | 25.42 |
| ATOM | 2192 | CB | ARG | A | 807 | −26.254 | 0.963 | 32.532 | 1.00 | 30.28 |
| ATOM | 2193 | CG | ARG | A | 807 | −25.516 | 1.919 | 33.450 | 1.00 | 31.10 |
| ATOM | 2194 | CD | ARG | A | 807 | −26.051 | 3.331 | 33.290 | 1.00 | 31.22 |
| ATOM | 2195 | NE | ARG | A | 807 | −26.092 | 3.752 | 31.892 | 1.00 | 31.51 |
| ATOM | 2196 | CZ | ARG | A | 807 | −25.040 | 4.157 | 31.192 | 1.00 | 31.00 |
| ATOM | 2197 | NH1 | ARG | A | 807 | −23.839 | 4.205 | 31.753 | 1.00 | 32.13 |
| ATOM | 2198 | NH2 | ARG | A | 807 | −25.191 | 4.516 | 29.923 | 1.00 | 30.26 |
| ATOM | 2199 | C | ARG | A | 807 | −26.528 | −1.030 | 34.002 | 1.00 | 27.00 |
| ATOM | 2200 | O | ARG | A | 807 | −25.916 | −0.988 | 35.073 | 1.00 | 26.42 |
| ATOM | 2201 | N | GLU | A | 808 | −27.762 | −1.508 | 33.895 | 1.00 | 29.47 |
| ATOM | 2202 | CA | GLU | A | 808 | −28.477 | −2.031 | 35.053 | 1.00 | 30.52 |
| ATOM | 2203 | CB | GLU | A | 808 | −29.914 | −2.372 | 34.659 | 1.00 | 44.58 |
| ATOM | 2204 | CG | GLU | A | 808 | −30.775 | −1.147 | 34.408 | 1.00 | 45.18 |
| ATOM | 2205 | CD | GLU | A | 808 | −32.132 | −1.492 | 33.837 | 1.00 | 45.51 |
| ATOM | 2206 | OE1 | GLU | A | 808 | −32.761 | −2.451 | 34.332 | 1.00 | 45.53 |
| ATOM | 2207 | OE2 | GLU | A | 808 | −32.573 | −0.796 | 32.898 | 1.00 | 45.65 |
| ATOM | 2208 | C | GLU | A | 808 | −27.780 | −3.261 | 35.629 | 1.00 | 30.44 |
| ATOM | 2209 | O | GLU | A | 808 | −27.875 | −3.542 | 36.828 | 1.00 | 30.33 |
| ATOM | 2210 | N | LYS | A | 809 | −27.077 | −3.985 | 34.765 | 1.00 | 31.29 |
| ATOM | 2211 | CA | LYS | A | 809 | −26.355 | −5.190 | 35.162 | 1.00 | 31.25 |
| ATOM | 2212 | CB | LYS | A | 809 | −26.522 | −6.279 | 34.092 | 1.00 | 41.16 |
| ATOM | 2213 | CG | LYS | A | 809 | −27.951 | −6.773 | 33.888 | 1.00 | 41.47 |
| ATOM | 2214 | CD | LYS | A | 809 | −28.391 | −7.716 | 34.995 | 1.00 | 41.55 |
| ATOM | 2215 | CE | LYS | A | 809 | −27.714 | −9.075 | 34.885 | 1.00 | 41.48 |
| ATOM | 2216 | NZ | LYS | A | 809 | −28.230 | −9.865 | 33.733 | 1.00 | 41.41 |
| ATOM | 2217 | C | LYS | A | 809 | −24.870 | −4.879 | 35.338 | 1.00 | 30.98 |
| ATOM | 2218 | O | LYS | A | 809 | −24.023 | −5.744 | 35.129 | 1.00 | 30.77 |
| ATOM | 2219 | N | LYS | A | 810 | −24.551 | −3.646 | 35.721 | 1.00 | 26.58 |
| ATOM | 2220 | CA | LYS | A | 810 | −23.155 | −3.259 | 35.901 | 1.00 | 26.99 |
| ATOM | 2221 | CB | LYS | A | 810 | −23.050 | −1.768 | 36.235 | 1.00 | 43.46 |
| ATOM | 2222 | CG | LYS | A | 810 | −23.626 | −1.387 | 37.587 | 1.00 | 44.21 |
| ATOM | 2223 | CD | LYS | A | 810 | −23.413 | 0.090 | 37.878 | 1.00 | 44.21 |
| ATOM | 2224 | CE | LYS | A | 810 | −23.942 | 0.459 | 39.256 | 1.00 | 44.19 |
| ATOM | 2225 | NZ | LYS | A | 810 | −23.718 | 1.897 | 39.566 | 1.00 | 44.47 |
| ATOM | 2226 | C | LYS | A | 810 | −22.462 | −4.077 | 36.997 | 1.00 | 26.09 |
| ATOM | 2227 | O | LYS | A | 810 | −21.234 | −4.128 | 37.060 | 1.00 | 26.59 |
| ATOM | 2228 | N | ASN | A | 811 | −23.250 | −4.720 | 37.852 | 1.00 | 29.32 |
| ATOM | 2229 | CA | ASN | A | 811 | −22.693 | −5.526 | 38.934 | 1.00 | 29.87 |
| ATOM | 2230 | CB | ASN | A | 811 | −23.812 | −6.017 | 39.858 | 1.00 | 45.97 |
| ATOM | 2231 | CG | ASN | A | 811 | −24.632 | −7.135 | 39.240 | 1.00 | 46.38 |
| ATOM | 2232 | OD1 | ASN | A | 811 | −24.137 | −8.247 | 39.042 | 1.00 | 46.84 |
| ATOM | 2233 | ND2 | ASN | A | 811 | −25.887 | −6.847 | 38.927 | 1.00 | 46.45 |
| ATOM | 2234 | C | ASN | A | 811 | −21.931 | −6.730 | 38.385 | 1.00 | 29.68 |
| ATOM | 2235 | O | ASN | A | 811 | −21.228 | −7.425 | 39.121 | 1.00 | 29.10 |
| ATOM | 2236 | N | LYS | A | 812 | −22.075 | −6.970 | 37.086 | 1.00 | 24.91 |
| ATOM | 2237 | CA | LYS | A | 812 | −21.418 | −8.100 | 36.438 | 1.00 | 25.57 |
| ATOM | 2238 | CB | LYS | A | 812 | −22.292 | −8.623 | 35.295 | 1.00 | 45.40 |
| ATOM | 2239 | CG | LYS | A | 812 | −22.850 | −10.016 | 35.509 | 1.00 | 46.25 |
| ATOM | 2240 | CD | LYS | A | 812 | −23.811 | −10.060 | 36.678 | 1.00 | 46.19 |
| ATOM | 2241 | CE | LYS | A | 812 | −24.429 | −11.439 | 36.816 | 1.00 | 46.23 |
| ATOM | 2242 | NZ | LYS | A | 812 | −23.387 | −12.487 | 37.001 | 1.00 | 46.38 |
| ATOM | 2243 | C | LYS | A | 812 | −20.025 | −7.789 | 35.886 | 1.00 | 24.76 |
| ATOM | 2244 | O | LYS | A | 812 | −19.340 | −8.688 | 35.401 | 1.00 | 24.68 |
| ATOM | 2245 | N | ILE | A | 813 | −19.598 | −6.534 | 35.963 | 1.00 | 26.65 |
| ATOM | 2246 | CA | ILE | A | 813 | −18.294 | −6.162 | 35.424 | 1.00 | 26.72 |
| ATOM | 2247 | CB | ILE | A | 813 | −18.008 | −4.656 | 35.630 | 1.00 | 25.86 |
| ATOM | 2248 | CG2 | ILE | A | 813 | −16.564 | −4.337 | 35.260 | 1.00 | 25.72 |
| ATOM | 2249 | CG1 | ILE | A | 813 | −18.955 | −3.830 | 34.751 | 1.00 | 25.85 |
| ATOM | 2250 | CD1 | ILE | A | 813 | −18.880 | −2.335 | 34.984 | 1.00 | 26.75 |
| ATOM | 2251 | C | ILE | A | 813 | −17.123 | −6.986 | 35.957 | 1.00 | 26.25 |
| ATOM | 2252 | O | ILE | A | 813 | −16.271 | −7.426 | 35.185 | 1.00 | 25.98 |
| ATOM | 2253 | N | PRO | A | 814 | −17.055 | −7.205 | 37.280 | 1.00 | 22.10 |
| ATOM | 2254 | CD | PRO | A | 814 | −17.742 | −6.559 | 38.415 | 1.00 | 23.22 |
| ATOM | 2255 | CA | PRO | A | 814 | −15.916 | −8.003 | 37.741 | 1.00 | 23.12 |
| ATOM | 2256 | CB | PRO | A | 814 | −16.116 | −8.048 | 39.253 | 1.00 | 22.48 |
| ATOM | 2257 | CG | PRO | A | 814 | −16.722 | −6.697 | 39.537 | 1.00 | 22.19 |
| ATOM | 2258 | C | PRO | A | 814 | −15.842 | −9.392 | 37.115 | 1.00 | 23.05 |
| ATOM | 2259 | O | PRO | A | 814 | −14.792 | −9.796 | 36.619 | 1.00 | 23.01 |
| ATOM | 2260 | N | SER | A | 815 | −16.952 | −10.123 | 37.129 | 1.00 | 24.56 |
| ATOM | 2261 | CA | SER | A | 815 | −16.955 | −11.465 | 36.555 | 1.00 | 24.61 |
| ATOM | 2262 | CB | SER | A | 815 | −18.286 | −12.174 | 36.844 | 1.00 | 32.62 |
| ATOM | 2263 | OG | SER | A | 815 | −19.393 | −11.454 | 36.330 | 1.00 | 33.36 |
| ATOM | 2264 | C | SER | A | 815 | −16.687 | −11.440 | 35.049 | 1.00 | 24.80 |
| ATOM | 2265 | O | SER | A | 815 | −16.055 | −12.349 | 34.513 | 1.00 | 25.10 |
| ATOM | 2266 | N | MET | A | 816 | −17.153 | −10.398 | 34.368 | 1.00 | 25.71 |
| ATOM | 2267 | CA | MET | A | 816 | −16.938 | −10.286 | 32.925 | 1.00 | 24.80 |
| ATOM | 2268 | CB | MET | A | 816 | −17.820 | −9.188 | 32.332 | 1.00 | 40.93 |
| ATOM | 2269 | CG | MET | A | 816 | −19.302 | −9.432 | 32.489 | 1.00 | 41.93 |
| ATOM | 2270 | SD | MET | A | 816 | −20.264 | −8.316 | 31.459 | 1.00 | 42.83 |
| ATOM | 2271 | CE | MET | A | 816 | −19.866 | −6.716 | 32.201 | 1.00 | 41.73 |
| ATOM | 2272 | C | MET | A | 816 | −15.479 | −10.000 | 32.585 | 1.00 | 24.22 |
| ATOM | 2273 | O | MET | A | 816 | −14.969 | −10.476 | 31.574 | 1.00 | 24.07 |
| ATOM | 2274 | N | GLN | A | 817 | −14.806 | −9.216 | 33.420 | 1.00 | 24.08 |
| ATOM | 2275 | CA | GLN | A | 817 | −13.403 | −8.909 | 33.182 | 1.00 | 22.68 |
| ATOM | 2276 | CB | GLN | A | 817 | −12.904 | −7.844 | 34.161 | 1.00 | 21.90 |
| ATOM | 2277 | CG | GLN | A | 817 | −13.287 | −6.415 | 33.786 | 1.00 | 21.57 |
| ATOM | 2278 | CD | GLN | A | 817 | −12.797 | −6.020 | 32.407 | 1.00 | 22.28 |
| ATOM | 2279 | OE1 | GLN | A | 817 | −13.389 | −6.392 | 31.396 | 1.00 | 21.33 |
| ATOM | 2280 | NE2 | GLN | A | 817 | −11.704 | −5.272 | 32.359 | 1.00 | 21.19 |
| ATOM | 2281 | C | GLN | A | 817 | −12.587 | −10.185 | 33.344 | 1.00 | 24.08 |
| ATOM | 2282 | O | GLN | A | 817 | −11.655 | −10.435 | 32.588 | 1.00 | 22.00 |
| ATOM | 2283 | N | VAL | A | 818 | −12.947 | −10.990 | 34.340 | 1.00 | 22.79 |
| ATOM | 2284 | CA | VAL | A | 818 | −12.262 | −12.256 | 34.582 | 1.00 | 23.81 |
| ATOM | 2285 | CB | VAL | A | 818 | −12.839 | −12.958 | 35.828 | 1.00 | 26.49 |
| ATOM | 2286 | CG1 | VAL | A | 818 | −12.225 | −14.338 | 35.983 | 1.00 | 26.65 |
| ATOM | 2287 | CG2 | VAL | A | 818 | −12.567 | −12.115 | 37.064 | 1.00 | 26.49 |
| ATOM | 2288 | C | VAL | A | 818 | −12.453 | −13.161 | 33.364 | 1.00 | 24.69 |
| ATOM | 2289 | O | VAL | A | 818 | −11.502 | −13.776 | 32.871 | 1.00 | 23.39 |
| ATOM | 2290 | N | GLY | A | 819 | −13.687 | −13.234 | 32.878 | 1.00 | 25.13 |
| ATOM | 2291 | CA | GLY | A | 819 | −13.971 | −14.061 | 31.716 | 1.00 | 24.79 |
| ATOM | 2292 | C | GLY | A | 819 | −13.221 | −13.571 | 30.490 | 1.00 | 24.86 |
| ATOM | 2293 | O | GLY | A | 819 | −12.729 | −14.354 | 29.677 | 1.00 | 25.43 |
| ATOM | 2294 | N | PHE | A | 820 | −13.140 | −12.255 | 30.355 | 1.00 | 24.15 |
| ATOM | 2295 | CA | PHE | A | 820 | −12.440 | −11.635 | 29.241 | 1.00 | 22.60 |
| ATOM | 2296 | CB | PHE | A | 820 | −12.606 | −10.113 | 29.336 | 1.00 | 22.65 |
| ATOM | 2297 | CG | PHE | A | 820 | −11.736 | −9.335 | 28.389 | 1.00 | 22.10 |
| ATOM | 2298 | CD1 | PHE | A | 820 | −11.863 | −9.490 | 27.011 | 1.00 | 22.63 |
| ATOM | 2299 | CD2 | PHE | A | 820 | −10.803 | −8.424 | 28.879 | 1.00 | 22.66 |
| ATOM | 2300 | CE1 | PHE | A | 820 | −11.069 | −8.743 | 26.133 | 1.00 | 22.28 |
| ATOM | 2301 | CE2 | PHE | A | 820 | −10.008 | −7.677 | 28.011 | 1.00 | 22.73 |
| ATOM | 2302 | CZ | PHE | A | 820 | −10.143 | −7.839 | 26.637 | 1.00 | 22.74 |
| ATOM | 2303 | C | PHE | A | 820 | −10.964 | −12.024 | 29.293 | 1.00 | 23.46 |
| ATOM | 2304 | O | PHE | A | 820 | −10.357 | −12.369 | 28.283 | 1.00 | 21.24 |
| ATOM | 2305 | N | ILE | A | 821 | −10.390 | −11.984 | 30.488 | 1.00 | 21.23 |
| ATOM | 2306 | CA | ILE | A | 821 | −8.990 | −12.323 | 30.646 | 1.00 | 21.89 |
| ATOM | 2307 | CB | ILE | A | 821 | −8.525 | −12.044 | 32.088 | 1.00 | 22.37 |
| ATOM | 2308 | CG2 | ILE | A | 821 | −7.130 | −12.596 | 32.315 | 1.00 | 22.42 |
| ATOM | 2309 | CG1 | ILE | A | 821 | −8.526 | −10.534 | 32.332 | 1.00 | 21.45 |
| ATOM | 2310 | CD1 | ILE | A | 821 | −8.377 | −10.160 | 33.777 | 1.00 | 23.29 |
| ATOM | 2311 | C | ILE | A | 821 | −8.744 | −13.784 | 30.292 | 1.00 | 22.08 |
| ATOM | 2312 | O | ILE | A | 821 | −7.864 | −14.081 | 29.488 | 1.00 | 24.60 |
| ATOM | 2313 | N | ASP | A | 822 | −9.528 | −14.687 | 30.873 | 1.00 | 31.25 |
| ATOM | 2314 | CA | ASP | A | 822 | −9.364 | −16.113 | 30.600 | 1.00 | 32.20 |
| ATOM | 2315 | CB | ASP | A | 822 | −10.379 | −16.950 | 31.390 | 1.00 | 31.68 |
| ATOM | 2316 | CG | ASP | A | 822 | −10.096 | −16.978 | 32.876 | 1.00 | 30.88 |
| ATOM | 2317 | OD1 | ASP | A | 822 | −8.923 | −16.812 | 33.265 | 1.00 | 31.47 |
| ATOM | 2318 | OD2 | ASP | A | 822 | −11.054 | −17.186 | 33.652 | 1.00 | 31.29 |
| ATOM | 2319 | C | ASP | A | 822 | −9.520 | −16.474 | 29.127 | 1.00 | 33.01 |
| ATOM | 2320 | O | ASP | A | 822 | −8.680 | −17.165 | 28.552 | 1.00 | 33.17 |
| ATOM | 2321 | N | ALA | A | 823 | −10.600 | −15.996 | 28.523 | 1.00 | 31.01 |
| ATOM | 2322 | CA | ALA | A | 823 | −10.912 | −16.309 | 27.131 | 1.00 | 30.74 |
| ATOM | 2323 | CB | ALA | A | 823 | −12.402 | −16.092 | 26.894 | 1.00 | 28.33 |
| ATOM | 2324 | C | ALA | A | 823 | −10.123 | −15.608 | 26.030 | 1.00 | 30.37 |
| ATOM | 2325 | O | ALA | A | 823 | −9.919 | −16.179 | 24.959 | 1.00 | 30.71 |
| ATOM | 2326 | N | ILE | A | 824 | −9.671 | −14.385 | 26.281 | 1.00 | 26.40 |
| ATOM | 2327 | CA | ILE | A | 824 | −8.968 | −13.624 | 25.250 | 1.00 | 25.53 |
| ATOM | 2328 | CB | ILE | A | 824 | −9.761 | −12.325 | 24.898 | 1.00 | 28.97 |
| ATOM | 2329 | CG2 | ILE | A | 824 | −8.978 | −11.487 | 23.899 | 1.00 | 29.02 |
| ATOM | 2330 | CG1 | ILE | A | 824 | −11.154 | −12.665 | 24.357 | 1.00 | 29.19 |
| ATOM | 2331 | CD1 | ILE | A | 824 | −11.151 | −13.353 | 23.013 | 1.00 | 29.99 |
| ATOM | 2332 | C | ILE | A | 824 | −7.537 | −13.185 | 25.546 | 1.00 | 27.07 |
| ATOM | 2333 | O | ILE | A | 824 | −6.639 | −13.358 | 24.722 | 1.00 | 26.42 |
| ATOM | 2334 | N | CYS | A | 825 | −7.324 | −12.617 | 26.726 | 1.00 | 24.64 |
| ATOM | 2335 | CA | CYS | A | 825 | −6.020 | −12.070 | 27.075 | 1.00 | 24.29 |
| ATOM | 2336 | CB | CYS | A | 825 | −6.207 | −11.003 | 28.145 | 1.00 | 22.55 |
| ATOM | 2337 | SG | CYS | A | 825 | −7.489 | −9.813 | 27.703 | 1.00 | 21.61 |
| ATOM | 2338 | C | CYS | A | 825 | −4.878 | −12.979 | 27.493 | 1.00 | 23.81 |
| ATOM | 2339 | O | CYS | A | 825 | −3.776 | −12.856 | 26.971 | 1.00 | 23.60 |
| ATOM | 2340 | N | LEU | A | 826 | −5.131 | −13.866 | 28.448 | 1.00 | 25.10 |
| ATOM | 2341 | CA | LEU | A | 826 | −4.098 | −14.761 | 28.955 | 1.00 | 26.70 |
| ATOM | 2342 | CB | LEU | A | 826 | −4.727 | −15.770 | 29.921 | 1.00 | 38.73 |
| ATOM | 2343 | CG | LEU | A | 826 | −4.228 | −15.764 | 31.370 | 1.00 | 39.48 |
| ATOM | 2344 | CD1 | LEU | A | 826 | −4.024 | −14.343 | 31.867 | 1.00 | 38.99 |
| ATOM | 2345 | CD2 | LEU | A | 826 | −5.230 | −16.498 | 32.243 | 1.00 | 39.26 |
| ATOM | 2346 | C | LEU | A | 826 | −3.301 | −15.492 | 27.873 | 1.00 | 25.78 |
| ATOM | 2347 | O | LEU | A | 826 | −2.072 | −15.427 | 27.854 | 1.00 | 26.14 |
| ATOM | 2348 | N | GLN | A | 827 | −3.993 | −16.179 | 26.972 | 1.00 | 26.44 |
| ATOM | 2349 | CA | GLN | A | 827 | −3.308 | −16.919 | 25.920 | 1.00 | 26.16 |
| ATOM | 2350 | CB | GLN | A | 827 | −4.313 | −17.689 | 25.065 | 1.00 | 31.94 |
| ATOM | 2351 | CG | GLN | A | 827 | −5.146 | −18.687 | 25.839 | 1.00 | 32.38 |
| ATOM | 2352 | CD | GLN | A | 827 | −6.176 | −19.374 | 24.967 | 1.00 | 33.15 |
| ATOM | 2353 | OE1 | GLN | A | 827 | −5.844 | −20.233 | 24.146 | 1.00 | 33.30 |
| ATOM | 2354 | NE2 | GLN | A | 827 | −7.437 | −18.989 | 25.130 | 1.00 | 33.01 |
| ATOM | 2355 | C | GLN | A | 827 | −2.483 | −16.005 | 25.034 | 1.00 | 26.24 |
| ATOM | 2356 | O | GLN | A | 827 | −1.394 | −16.372 | 24.593 | 1.00 | 26.27 |
| ATOM | 2357 | N | LEU | A | 828 | −2.995 | −14.811 | 24.761 | 1.00 | 23.62 |
| ATOM | 2358 | CA | LEU | A | 828 | −2.256 | −13.887 | 23.912 | 1.00 | 23.54 |
| ATOM | 2359 | CB | LEU | A | 828 | −3.121 | −12.678 | 23.547 | 1.00 | 27.47 |
| ATOM | 2360 | CG | LEU | A | 828 | −2.393 | −11.621 | 22.705 | 1.00 | 27.18 |
| ATOM | 2361 | CD1 | LEU | A | 828 | −1.769 | −12.267 | 21.474 | 1.00 | 28.21 |
| ATOM | 2362 | CD2 | LEU | A | 828 | −3.361 | −10.534 | 22.293 | 1.00 | 27.24 |
| ATOM | 2363 | C | LEU | A | 828 | −0.948 | −13.398 | 24.525 | 1.00 | 24.07 |
| ATOM | 2364 | O | LEU | A | 828 | 0.089 | −13.389 | 23.863 | 1.00 | 23.90 |
| ATOM | 2365 | N | TYR | A | 829 | −0.984 | −12.979 | 25.785 | 1.00 | 22.58 |
| ATOM | 2366 | CA | TYR | A | 829 | 0.232 | −12.492 | 26.410 | 1.00 | 23.51 |
| ATOM | 2367 | CB | TYR | A | 829 | −0.108 | −11.655 | 27.652 | 1.00 | 22.13 |
| ATOM | 2368 | CG | TYR | A | 829 | −0.797 | −10.352 | 27.273 | 1.00 | 22.65 |
| ATOM | 2369 | CD1 | TYR | A | 829 | −0.133 | −9.395 | 26.504 | 1.00 | 21.70 |
| ATOM | 2370 | CE1 | TYR | A | 829 | −0.778 | −8.224 | 26.084 | 1.00 | 22.06 |
| ATOM | 2371 | CD2 | TYR | A | 829 | −2.126 | −10.110 | 27.622 | 1.00 | 22.96 |
| ATOM | 2372 | CE2 | TYR | A | 829 | −2.782 | −8.939 | 27.202 | 1.00 | 21.69 |
| ATOM | 2373 | CZ | TYR | A | 829 | −2.096 | −8.009 | 26.437 | 1.00 | 22.78 |
| ATOM | 2374 | OH | TYR | A | 829 | −2.717 | −6.850 | 26.023 | 1.00 | 20.96 |
| ATOM | 2375 | C | TYR | A | 829 | 1.171 | −13.653 | 26.725 | 1.00 | 22.44 |
| ATOM | 2376 | O | TYR | A | 829 | 2.378 | −13.469 | 26.803 | 1.00 | 23.40 |
| ATOM | 2377 | N | GLU | A | 830 | 0.615 | −14.851 | 26.877 | 1.00 | 26.33 |
| ATOM | 2378 | CA | GLU | A | 830 | 1.451 | −16.025 | 27.127 | 1.00 | 27.63 |
| ATOM | 2379 | CB | GLU | A | 830 | 0.602 | −17.227 | 27.539 | 1.00 | 34.99 |
| ATOM | 2380 | CG | GLU | A | 830 | 0.034 | −17.110 | 28.941 | 1.00 | 35.86 |
| ATOM | 2381 | CD | GLU | A | 830 | −0.865 | −18.266 | 29.318 | 1.00 | 36.17 |
| ATOM | 2382 | OE1 | GLU | A | 830 | −1.246 | −18.349 | 30.507 | 1.00 | 35.89 |
| ATOM | 2383 | OE2 | GLU | A | 830 | −1.195 | −19.084 | 28.430 | 1.00 | 36.49 |
| ATOM | 2384 | C | GLU | A | 830 | 2.186 | −16.326 | 25.826 | 1.00 | 26.91 |
| ATOM | 2385 | O | GLU | A | 830 | 3.383 | −16.614 | 25.827 | 1.00 | 27.95 |
| ATOM | 2386 | N | ALA | A | 831 | 1.465 | −16.235 | 24.711 | 1.00 | 26.22 |
| ATOM | 2387 | CA | ALA | A | 831 | 2.056 | −16.489 | 23.398 | 1.00 | 26.36 |
| ATOM | 2388 | CB | ALA | A | 831 | 0.965 | −16.533 | 22.328 | 1.00 | 20.71 |
| ATOM | 2389 | C | ALA | A | 831 | 3.091 | −15.425 | 23.044 | 1.00 | 25.92 |
| ATOM | 2390 | O | ALA | A | 831 | 4.108 | −15.725 | 22.429 | 1.00 | 26.14 |
| ATOM | 2391 | N | LEU | A | 832 | 2.833 | −14.180 | 23.432 | 1.00 | 25.86 |
| ATOM | 2392 | CA | LEU | A | 832 | 3.767 | −13.096 | 23.138 | 1.00 | 24.48 |
| ATOM | 2393 | CB | LEU | A | 832 | 3.127 | −11.738 | 23.447 | 1.00 | 23.29 |
| ATOM | 2394 | CG | LEU | A | 832 | 4.036 | −10.513 | 23.333 | 1.00 | 22.75 |
| ATOM | 2395 | CD1 | LEU | A | 832 | 4.597 | −10.387 | 21.926 | 1.00 | 22.85 |
| ATOM | 2396 | CD2 | LEU | A | 832 | 3.237 | −9.268 | 23.701 | 1.00 | 23.24 |
| ATOM | 2397 | C | LEU | A | 832 | 5.056 | −13.238 | 23.936 | 1.00 | 25.65 |
| ATOM | 2398 | O | LEU | A | 832 | 6.140 | −12.902 | 23.454 | 1.00 | 25.17 |
| ATOM | 2399 | N | THR | A | 833 | 4.931 | −13.720 | 25.166 | 1.00 | 26.35 |
| ATOM | 2400 | CA | THR | A | 833 | 6.092 | −13.910 | 26.021 | 1.00 | 27.50 |
| ATOM | 2401 | CB | THR | A | 833 | 5.654 | −14.289 | 27.446 | 1.00 | 28.58 |
| ATOM | 2402 | OG1 | THR | A | 833 | 4.872 | −13.218 | 27.994 | 1.00 | 28.28 |
| ATOM | 2403 | CG2 | THR | A | 833 | 6.868 | −14.531 | 28.339 | 1.00 | 28.51 |
| ATOM | 2404 | C | THR | A | 833 | 6.968 | −15.007 | 25.418 | 1.00 | 28.22 |
| ATOM | 2405 | O | THR | A | 833 | 8.197 | −14.977 | 25.540 | 1.00 | 28.01 |
| ATOM | 2406 | N | HIS | A | 834 | 6.333 | −15.964 | 24.750 | 1.00 | 32.53 |
| ATOM | 2407 | CA | HIS | A | 834 | 7.080 | −17.044 | 24.117 | 1.00 | 33.13 |
| ATOM | 2408 | CB | HIS | A | 834 | 6.131 | −18.053 | 23.473 | 1.00 | 41.03 |
| ATOM | 2409 | CG | HIS | A | 834 | 6.833 | −19.200 | 22.820 | 1.00 | 41.66 |
| ATOM | 2410 | CD2 | HIS | A | 834 | 7.017 | −19.505 | 21.514 | 1.00 | 41.45 |
| ATOM | 2411 | ND1 | HIS | A | 834 | 7.497 | −20.170 | 23.538 | 1.00 | 41.66 |
| ATOM | 2412 | CE1 | HIS | A | 834 | 8.060 | −21.024 | 22.702 | 1.00 | 41.76 |
| ATOM | 2413 | NE2 | HIS | A | 834 | 7.785 | −20.643 | 21.467 | 1.00 | 41.58 |
| ATOM | 2414 | C | HIS | A | 834 | 7.979 | −16.433 | 23.050 | 1.00 | 33.02 |
| ATOM | 2415 | O | HIS | A | 834 | 9.179 | −16.701 | 23.003 | 1.00 | 33.90 |
| ATOM | 2416 | N | VAL | A | 835 | 7.390 | −15.603 | 22.195 | 1.00 | 34.00 |
| ATOM | 2417 | CA | VAL | A | 835 | 8.142 | −14.943 | 21.136 | 1.00 | 33.47 |
| ATOM | 2418 | CB | VAL | A | 835 | 7.235 | −14.027 | 20.282 | 1.00 | 34.13 |
| ATOM | 2419 | CG1 | VAL | A | 835 | 8.082 | −13.226 | 19.304 | 1.00 | 33.51 |
| ATOM | 2420 | CG2 | VAL | A | 835 | 6.213 | −14.863 | 19.533 | 1.00 | 33.76 |
| ATOM | 2421 | C | VAL | A | 835 | 9.278 | −14.105 | 21.705 | 1.00 | 34.04 |
| ATOM | 2422 | O | VAL | A | 835 | 10.373 | −14.076 | 21.147 | 1.00 | 33.58 |
| ATOM | 2423 | N | SER | A | 836 | 9.017 | −13.416 | 22.813 | 1.00 | 31.52 |
| ATOM | 2424 | CA | SER | A | 836 | 10.041 | −12.585 | 23.436 | 1.00 | 32.24 |
| ATOM | 2425 | CB | SER | A | 836 | 10.016 | −11.176 | 22.838 | 1.00 | 33.50 |
| ATOM | 2426 | OG | SER | A | 836 | 11.125 | −10.416 | 23.284 | 1.00 | 33.58 |
| ATOM | 2427 | C | SER | A | 836 | 9.861 | −12.503 | 24.949 | 1.00 | 31.70 |
| ATOM | 2428 | O | SER | A | 836 | 8.937 | −11.856 | 25.447 | 1.00 | 30.73 |
| ATOM | 2429 | N | GLU | A | 837 | 10.758 | −13.168 | 25.668 | 1.00 | 32.13 |
| ATOM | 2430 | CA | GLU | A | 837 | 10.742 | −13.209 | 27.126 | 1.00 | 32.75 |
| ATOM | 2431 | CB | GLU | A | 837 | 12.035 | −13.862 | 27.624 | 1.00 | 50.88 |
| ATOM | 2432 | CG | GLU | A | 837 | 13.295 | −13.194 | 27.082 | 1.00 | 51.44 |
| ATOM | 2433 | CD | GLU | A | 837 | 14.564 | −13.981 | 27.368 | 1.00 | 51.73 |
| ATOM | 2434 | OE1 | GLU | A | 837 | 14.863 | −14.226 | 28.558 | 1.00 | 51.76 |
| ATOM | 2435 | OE2 | GLU | A | 837 | 15.264 | −14.349 | 26.399 | 1.00 | 51.58 |
| ATOM | 2436 | C | GLU | A | 837 | 10.579 | −11.836 | 27.779 | 1.00 | 32.06 |
| ATOM | 2437 | O | GLU | A | 837 | 9.916 | −11.702 | 28.808 | 1.00 | 32.57 |
| ATOM | 2438 | N | ASP | A | 838 | 11.185 | −10.821 | 27.175 | 1.00 | 27.93 |
| ATOM | 2439 | CA | ASP | A | 838 | 11.135 | −9.463 | 27.699 | 1.00 | 28.49 |
| ATOM | 2440 | CB | ASP | A | 838 | 12.085 | −8.574 | 26.896 | 1.00 | 35.78 |
| ATOM | 2441 | CG | ASP | A | 838 | 13.511 | −9.093 | 26.908 | 1.00 | 37.30 |
| ATOM | 2442 | OD1 | ASP | A | 838 | 14.167 | −9.014 | 27.969 | 1.00 | 36.40 |
| ATOM | 2443 | OD2 | ASP | A | 838 | 13.971 | −9.589 | 25.857 | 1.00 | 37.49 |
| ATOM | 2444 | C | ASP | A | 838 | 9.738 | −8.838 | 27.723 | 1.00 | 27.26 |
| ATOM | 2445 | O | ASP | A | 838 | 9.570 | −7.716 | 28.202 | 1.00 | 27.48 |
| ATOM | 2446 | N | CYS | A | 839 | 8.740 | −9.550 | 27.207 | 1.00 | 26.97 |
| ATOM | 2447 | CA | CYS | A | 839 | 7.372 | −9.035 | 27.207 | 1.00 | 26.65 |
| ATOM | 2448 | CB | CYS | A | 839 | 6.668 | −9.360 | 25.882 | 1.00 | 26.64 |
| ATOM | 2449 | SG | CYS | A | 839 | 7.139 | −8.301 | 24.485 | 1.00 | 26.65 |
| ATOM | 2450 | C | CYS | A | 839 | 6.586 | −9.638 | 28.365 | 1.00 | 27.57 |
| ATOM | 2451 | O | CYS | A | 839 | 5.373 | −9.461 | 28.471 | 1.00 | 26.86 |
| ATOM | 2452 | N | PHE | A | 840 | 7.282 | −10.352 | 29.243 | 1.00 | 26.10 |
| ATOM | 2453 | CA | PHE | A | 840 | 6.626 | −10.976 | 30.385 | 1.00 | 26.52 |
| ATOM | 2454 | CB | PHE | A | 840 | 7.653 | −11.679 | 31.281 | 1.00 | 35.43 |
| ATOM | 2455 | CG | PHE | A | 840 | 7.045 | −12.344 | 32.485 | 1.00 | 35.52 |
| ATOM | 2456 | CD1 | PHE | A | 840 | 6.211 | −13.450 | 32.340 | 1.00 | 35.69 |
| ATOM | 2457 | CD2 | PHE | A | 840 | 7.273 | −11.842 | 33.762 | 1.00 | 35.56 |
| ATOM | 2458 | CE1 | PHE | A | 840 | 5.610 | −14.046 | 33.450 | 1.00 | 35.99 |
| ATOM | 2459 | CE2 | PHE | A | 840 | 6.678 | −12.429 | 34.878 | 1.00 | 35.60 |
| ATOM | 2460 | CZ | PHE | A | 840 | 5.843 | −13.533 | 34.722 | 1.00 | 35.62 |
| ATOM | 2461 | C | PHE | A | 840 | 5.810 | −9.991 | 31.227 | 1.00 | 25.53 |
| ATOM | 2462 | O | PHE | A | 840 | 4.726 | −10.332 | 31.698 | 1.00 | 26.75 |
| ATOM | 2463 | N | PRO | A | 841 | 6.311 | −8.757 | 31.422 | 1.00 | 24.00 |
| ATOM | 2464 | CD | PRO | A | 841 | 7.585 | −8.177 | 30.965 | 1.00 | 27.23 |
| ATOM | 2465 | CA | PRO | A | 841 | 5.567 | −7.783 | 32.225 | 1.00 | 23.95 |
| ATOM | 2466 | CB | PRO | A | 841 | 6.345 | −6.491 | 32.002 | 1.00 | 26.33 |
| ATOM | 2467 | CG | PRO | A | 841 | 7.751 | −6.988 | 31.890 | 1.00 | 27.03 |
| ATOM | 2468 | C | PRO | A | 841 | 4.104 | −7.662 | 31.821 | 1.00 | 24.02 |
| ATOM | 2469 | O | PRO | A | 841 | 3.238 | −7.446 | 32.667 | 1.00 | 23.76 |
| ATOM | 2470 | N | LEU | A | 842 | 3.826 | −7.809 | 30.531 | 1.00 | 23.41 |
| ATOM | 2471 | CA | LEU | A | 842 | 2.447 | −7.711 | 30.069 | 1.00 | 22.32 |
| ATOM | 2472 | CB | LEU | A | 842 | 2.400 | −7.647 | 28.540 | 1.00 | 22.25 |
| ATOM | 2473 | CG | LEU | A | 842 | 3.064 | −6.397 | 27.957 | 1.00 | 22.19 |
| ATOM | 2474 | CD1 | LEU | A | 842 | 3.184 | −6.527 | 26.442 | 1.00 | 21.97 |
| ATOM | 2475 | CD2 | LEU | A | 842 | 2.255 | −5.154 | 28.349 | 1.00 | 23.44 |
| ATOM | 2476 | C | LEU | A | 842 | 1.633 | −8.888 | 30.590 | 1.00 | 23.15 |
| ATOM | 2477 | O | LEU | A | 842 | 0.508 | −8.714 | 31.048 | 1.00 | 22.28 |
| ATOM | 2478 | N | LEU | A | 843 | 2.210 | −10.085 | 30.537 | 1.00 | 20.37 |
| ATOM | 2479 | CA | LEU | A | 843 | 1.528 | −11.277 | 31.019 | 1.00 | 22.54 |
| ATOM | 2480 | CB | LEU | A | 843 | 2.369 | −12.517 | 30.716 | 1.00 | 30.62 |
| ATOM | 2481 | CG | LEU | A | 843 | 1.666 | −13.867 | 30.542 | 1.00 | 32.10 |
| ATOM | 2482 | CD1 | LEU | A | 843 | 2.718 | −14.960 | 30.647 | 1.00 | 31.42 |
| ATOM | 2483 | CD2 | LEU | A | 843 | 0.586 | −14.078 | 31.579 | 1.00 | 31.97 |
| ATOM | 2484 | C | LEU | A | 843 | 1.338 | −11.162 | 32.531 | 1.00 | 20.83 |
| ATOM | 2485 | O | LEU | A | 843 | 0.252 | −11.406 | 33.057 | 1.00 | 22.49 |
| ATOM | 2486 | N | ASP | A | 844 | 2.410 | −10.791 | 33.222 | 1.00 | 25.10 |
| ATOM | 2487 | CA | ASP | A | 844 | 2.381 | −10.650 | 34.673 | 1.00 | 25.84 |
| ATOM | 2488 | CB | ASP | A | 844 | 3.756 | −10.204 | 35.177 | 1.00 | 33.41 |
| ATOM | 2489 | CG | ASP | A | 844 | 3.889 | −10.316 | 36.680 | 1.00 | 33.77 |
| ATOM | 2490 | OD1 | ASP | A | 844 | 3.499 | −11.367 | 37.233 | 1.00 | 33.62 |
| ATOM | 2491 | OD2 | ASP | A | 844 | 4.392 | −9.360 | 37.305 | 1.00 | 34.08 |
| ATOM | 2492 | C | ASP | A | 844 | 1.306 | −9.667 | 35.129 | 1.00 | 25.11 |
| ATOM | 2493 | O | ASP | A | 844 | 0.517 | −9.976 | 36.020 | 1.00 | 25.50 |
| ATOM | 2494 | N | GLY | A | 845 | 1.272 | −8.492 | 34.505 | 1.00 | 23.99 |
| ATOM | 2495 | CA | GLY | A | 845 | 0.282 | −7.488 | 34.861 | 1.00 | 24.47 |
| ATOM | 2496 | C | GLY | A | 845 | −1.141 | −7.954 | 34.612 | 1.00 | 24.36 |
| ATOM | 2497 | O | GLY | A | 845 | −2.057 | −7.628 | 35.366 | 1.00 | 23.75 |
| ATOM | 2498 | N | CYS | A | 846 | −1.325 | −8.720 | 33.542 | 1.00 | 23.31 |
| ATOM | 2499 | CA | CYS | A | 846 | −2.631 | −9.252 | 33.188 | 1.00 | 22.27 |
| ATOM | 2500 | CB | CYS | A | 846 | −2.556 | −9.961 | 31.833 | 1.00 | 24.57 |
| ATOM | 2501 | SG | CYS | A | 846 | −4.118 | −10.668 | 31.278 | 1.00 | 23.77 |
| ATOM | 2502 | C | CYS | A | 846 | −3.093 | −10.236 | 34.255 | 1.00 | 23.19 |
| ATOM | 2503 | O | CYS | A | 846 | −4.237 | −10.197 | 34.699 | 1.00 | 23.71 |
| ATOM | 2504 | N | ARG | A | 847 | −2.197 | −11.126 | 34.669 | 1.00 | 25.45 |
| ATOM | 2505 | CA | ARG | A | 847 | −2.554 | −12.103 | 35.681 | 1.00 | 26.33 |
| ATOM | 2506 | CB | ARG | A | 847 | −1.454 | −13.170 | 35.788 | 1.00 | 44.92 |
| ATOM | 2507 | CG | ARG | A | 847 | −1.334 | −13.973 | 34.489 | 1.00 | 45.65 |
| ATOM | 2508 | CD | ARG | A | 847 | −0.520 | −15.257 | 34.590 | 1.00 | 45.89 |
| ATOM | 2509 | NE | ARG | A | 847 | 0.924 | −15.037 | 34.557 | 1.00 | 45.99 |
| ATOM | 2510 | CZ | ARG | A | 847 | 1.805 | −15.950 | 34.155 | 1.00 | 45.73 |
| ATOM | 2511 | NH1 | ARG | A | 847 | 1.392 | −17.142 | 33.743 | 1.00 | 45.92 |
| ATOM | 2512 | NH2 | ARG | A | 847 | 3.102 | −15.678 | 34.178 | 1.00 | 45.71 |
| ATOM | 2513 | C | ARG | A | 847 | −2.839 | −11.434 | 37.021 | 1.00 | 25.65 |
| ATOM | 2514 | O | ARG | A | 847 | −3.673 | −11.909 | 37.794 | 1.00 | 25.84 |
| ATOM | 2515 | N | LYS | A | 848 | −2.172 | −10.319 | 37.291 | 1.00 | 27.18 |
| ATOM | 2516 | CA | LYS | A | 848 | −2.408 | −9.606 | 38.540 | 1.00 | 27.41 |
| ATOM | 2517 | CB | LYS | A | 848 | −1.344 | −8.529 | 38.755 | 1.00 | 29.68 |
| ATOM | 2518 | CG | LYS | A | 848 | 0.011 | −9.082 | 39.165 | 1.00 | 30.06 |
| ATOM | 2519 | CD | LYS | A | 848 | 1.023 | −7.964 | 39.328 | 1.00 | 30.03 |
| ATOM | 2520 | CE | LYS | A | 848 | 2.406 | −8.518 | 39.644 | 1.00 | 30.39 |
| ATOM | 2521 | NZ | LYS | A | 848 | 3.450 | −7.453 | 39.583 | 1.00 | 30.98 |
| ATOM | 2522 | C | LYS | A | 848 | −3.796 | −8.974 | 38.514 | 1.00 | 27.81 |
| ATOM | 2523 | O | LYS | A | 848 | −4.515 | −8.996 | 39.511 | 1.00 | 28.26 |
| ATOM | 2524 | N | ASN | A | 849 | −4.179 | −8.412 | 37.372 | 1.00 | 28.20 |
| ATOM | 2525 | CA | ASN | A | 849 | −5.498 | −7.807 | 37.270 | 1.00 | 26.64 |
| ATOM | 2526 | CB | ASN | A | 849 | −5.640 | −7.028 | 35.961 | 1.00 | 26.08 |
| ATOM | 2527 | CG | ASN | A | 849 | −4.970 | −5.674 | 36.027 | 1.00 | 25.73 |
| ATOM | 2528 | OD1 | ASN | A | 849 | −4.993 | −5.012 | 37.067 | 1.00 | 26.13 |
| ATOM | 2529 | ND2 | ASN | A | 849 | −4.382 | −5.247 | 34.923 | 1.00 | 26.48 |
| ATOM | 2530 | C | ASN | A | 849 | −6.572 | −8.875 | 37.372 | 1.00 | 27.18 |
| ATOM | 2531 | O | ASN | A | 849 | −7.679 | −8.612 | 37.844 | 1.00 | 26.84 |
| ATOM | 2532 | N | ARG | A | 850 | −6.244 | −10.086 | 36.934 | 1.00 | 25.96 |
| ATOM | 2533 | CA | ARG | A | 850 | −7.194 | −11.177 | 37.017 | 1.00 | 26.10 |
| ATOM | 2534 | CB | ARG | A | 850 | −6.640 | −12.431 | 36.338 | 1.00 | 38.03 |
| ATOM | 2535 | CG | ARG | A | 850 | −7.583 | −13.607 | 36.453 | 1.00 | 38.77 |
| ATOM | 2536 | CD | ARG | A | 850 | −6.976 | −14.898 | 35.941 | 1.00 | 38.89 |
| ATOM | 2537 | NE | ARG | A | 850 | −7.798 | −16.037 | 36.336 | 1.00 | 39.62 |
| ATOM | 2538 | CZ | ARG | A | 850 | −9.075 | −16.181 | 36.003 | 1.00 | 39.00 |
| ATOM | 2539 | NH1 | ARG | A | 850 | −9.669 | −15.255 | 35.264 | 1.00 | 40.11 |
| ATOM | 2540 | NH2 | ARG | A | 850 | −9.765 | −17.235 | 36.417 | 1.00 | 38.97 |
| ATOM | 2541 | C | ARG | A | 850 | −7.513 | −11.488 | 38.480 | 1.00 | 25.71 |
| ATOM | 2542 | O | ARG | A | 850 | −8.675 | −11.647 | 38.849 | 1.00 | 26.96 |
| ATOM | 2543 | N | GLN | A | 851 | −6.476 | −11.564 | 39.311 | 1.00 | 29.93 |
| ATOM | 2544 | CA | GLN | A | 851 | −6.659 | −11.860 | 40.728 | 1.00 | 30.30 |
| ATOM | 2545 | CB | GLN | A | 851 | −5.294 | −11.993 | 41.413 | 1.00 | 53.42 |
| ATOM | 2546 | CG | GLN | A | 851 | −4.938 | −10.873 | 42.375 | 1.00 | 54.29 |
| ATOM | 2547 | CD | GLN | A | 851 | −3.479 | −10.910 | 42.794 | 1.00 | 54.54 |
| ATOM | 2548 | OE1 | GLN | A | 851 | −3.071 | −10.204 | 43.717 | 1.00 | 54.48 |
| ATOM | 2549 | NE2 | GLN | A | 851 | −2.681 | −11.724 | 42.106 | 1.00 | 54.48 |
| ATOM | 2550 | C | GLN | A | 851 | −7.493 | −10.765 | 41.381 | 1.00 | 29.94 |
| ATOM | 2551 | O | GLN | A | 851 | −8.377 | −11.042 | 42.189 | 1.00 | 30.04 |
| ATOM | 2552 | N | LYS | A | 852 | −7.213 | −9.518 | 41.017 | 1.00 | 27.54 |
| ATOM | 2553 | CA | LYS | A | 852 | −7.950 | −8.381 | 41.557 | 1.00 | 27.65 |
| ATOM | 2554 | CB | LYS | A | 852 | −7.378 | −7.075 | 40.998 | 1.00 | 40.34 |
| ATOM | 2555 | CG | LYS | A | 852 | −6.005 | −6.705 | 41.527 | 1.00 | 40.79 |
| ATOM | 2556 | CD | LYS | A | 852 | −6.096 | −6.263 | 42.977 | 1.00 | 40.88 |
| ATOM | 2557 | CE | LYS | A | 852 | −4.760 | −5.766 | 43.497 | 1.00 | 40.95 |
| ATOM | 2558 | NZ | LYS | A | 852 | −4.883 | −5.208 | 44.879 | 1.00 | 41.45 |
| ATOM | 2559 | C | LYS | A | 852 | −9.433 | −8.470 | 41.204 | 1.00 | 27.70 |
| ATOM | 2560 | O | LYS | A | 852 | −10.301 | −8.360 | 42.075 | 1.00 | 27.95 |
| ATOM | 2561 | N | TRP | A | 853 | −9.722 | −8.655 | 39.920 | 1.00 | 24.75 |
| ATOM | 2562 | CA | TRP | A | 853 | −11.103 | −8.747 | 39.467 | 1.00 | 25.36 |
| ATOM | 2563 | CB | TRP | A | 853 | −11.150 | −8.857 | 37.940 | 1.00 | 23.18 |
| ATOM | 2564 | CG | TRP | A | 853 | −10.967 | −7.544 | 37.240 | 1.00 | 23.11 |
| ATOM | 2565 | CD2 | TRP | A | 853 | −11.771 | −6.368 | 37.399 | 1.00 | 22.95 |
| ATOM | 2566 | CE2 | TRP | A | 853 | −11.244 | −5.382 | 36.534 | 1.00 | 22.63 |
| ATOM | 2567 | CE3 | TRP | A | 853 | −12.886 | −6.052 | 38.189 | 1.00 | 23.00 |
| ATOM | 2568 | CD1 | TRP | A | 853 | −10.013 | −7.233 | 36.312 | 1.00 | 23.06 |
| ATOM | 2569 | NE1 | TRP | A | 853 | −10.173 | −5.937 | 35.884 | 1.00 | 22.64 |
| ATOM | 2570 | CZ2 | TRP | A | 853 | −11.794 | −4.101 | 36.436 | 1.00 | 22.97 |
| ATOM | 2571 | CZ3 | TRP | A | 853 | −13.433 | −4.778 | 38.092 | 1.00 | 22.86 |
| ATOM | 2572 | CH2 | TRP | A | 853 | −12.886 | −3.818 | 37.220 | 1.00 | 22.99 |
| ATOM | 2573 | C | TRP | A | 853 | −11.816 | −9.931 | 40.101 | 1.00 | 24.85 |
| ATOM | 2574 | O | TRP | A | 853 | −12.976 | −9.824 | 40.505 | 1.00 | 24.94 |
| ATOM | 2575 | N | GLN | A | 854 | −11.107 | −11.051 | 40.202 | 1.00 | 28.90 |
| ATOM | 2576 | CA | GLN | A | 854 | −11.662 | −12.264 | 40.785 | 1.00 | 30.44 |
| ATOM | 2577 | CB | GLN | A | 854 | −10.673 | −13.419 | 40.625 | 1.00 | 37.29 |
| ATOM | 2578 | CG | GLN | A | 854 | −11.183 | −14.749 | 41.152 | 1.00 | 37.88 |
| ATOM | 2579 | CD | GLN | A | 854 | −12.554 | −15.102 | 40.608 | 1.00 | 38.02 |
| ATOM | 2580 | OE1 | GLN | A | 854 | −12.812 | −14.978 | 39.411 | 1.00 | 38.44 |
| ATOM | 2581 | NE2 | GLN | A | 854 | −13.439 | −15.557 | 41.487 | 1.00 | 38.37 |
| ATOM | 2582 | C | GLN | A | 854 | −12.014 | −12.081 | 42.257 | 1.00 | 30.52 |
| ATOM | 2583 | O | GLN | A | 854 | −12.994 | −12.650 | 42.736 | 1.00 | 31.44 |
| ATOM | 2584 | N | ALA | A | 855 | −11.219 | −11.288 | 42.969 | 1.00 | 32.15 |
| ATOM | 2585 | CA | ALA | A | 855 | −11.465 | −11.039 | 44.388 | 1.00 | 32.96 |
| ATOM | 2586 | CB | ALA | A | 855 | −10.282 | −10.300 | 45.003 | 1.00 | 27.28 |
| ATOM | 2587 | C | ALA | A | 855 | −12.747 | −10.231 | 44.572 | 1.00 | 33.38 |
| ATOM | 2588 | O | ALA | A | 855 | −13.473 | −10.411 | 45.551 | 1.00 | 33.79 |
| ATOM | 2589 | N | LEU | A | 856 | −13.020 | −9.338 | 43.627 | 1.00 | 34.25 |
| ATOM | 2590 | CA | LEU | A | 856 | −14.223 | −8.517 | 43.685 | 1.00 | 34.81 |
| ATOM | 2591 | CB | LEU | A | 856 | −14.102 | −7.325 | 42.732 | 1.00 | 29.75 |
| ATOM | 2592 | CG | LEU | A | 856 | −12.988 | −6.310 | 43.001 | 1.00 | 29.61 |
| ATOM | 2593 | CD1 | LEU | A | 856 | −12.998 | −5.251 | 41.926 | 1.00 | 29.93 |
| ATOM | 2594 | CD2 | LEU | A | 856 | −13.180 | −5.679 | 44.373 | 1.00 | 29.63 |
| ATOM | 2595 | C | LEU | A | 856 | −15.455 | −9.339 | 43.315 | 1.00 | 35.51 |
| ATOM | 2596 | O | LEU | A | 856 | −16.560 | −9.063 | 43.786 | 1.00 | 35.20 |
| ATOM | 2597 | N | ALA | A | 857 | −15.258 | −10.358 | 42.484 | 1.00 | 39.71 |
| ATOM | 2598 | CA | ALA | A | 857 | −16.354 | −11.208 | 42.030 | 1.00 | 40.78 |
| ATOM | 2599 | CB | ALA | A | 857 | −16.044 | −11.745 | 40.640 | 1.00 | 32.69 |
| ATOM | 2600 | C | ALA | A | 857 | −16.710 | −12.369 | 42.956 | 1.00 | 41.78 |
| ATOM | 2601 | O | ALA | A | 857 | −17.849 | −12.833 | 42.951 | 1.00 | 41.70 |
| ATOM | 2602 | N | GLU | A | 858 | −15.748 | −12.847 | 43.738 | 1.00 | 51.08 |
| ATOM | 2603 | CA | GLU | A | 858 | −16.005 | −13.967 | 44.639 | 1.00 | 52.63 |
| ATOM | 2604 | CB | GLU | A | 858 | −14.710 | −14.741 | 44.909 | 1.00 | 56.18 |
| ATOM | 2605 | CG | GLU | A | 858 | −13.570 | −13.890 | 45.433 | 1.00 | 56.32 |
| ATOM | 2606 | CD | GLU | A | 858 | −12.341 | −14.710 | 45.778 | 1.00 | 56.60 |
| ATOM | 2607 | OE1 | GLU | A | 858 | −11.844 | −15.446 | 44.898 | 1.00 | 56.57 |
| ATOM | 2608 | OE2 | GLU | A | 858 | −11.871 | −14.614 | 46.930 | 1.00 | 56.45 |
| ATOM | 2609 | C | GLU | A | 858 | −16.635 | −13.536 | 45.960 | 1.00 | 53.20 |
| ATOM | 2610 | O | GLU | A | 858 | −15.986 | −13.709 | 47.014 | 1.00 | 53.41 |
| ATOM | 2611 | OXT | GLU | A | 858 | −17.776 | −13.032 | 45.927 | 1.00 | 56.42 |
| TER | | | | | | | | | | |
| ATOM | 2613 | CB | THR | B | 537 | 25.255 | 66.687 | 12.776 | 1.00 | 47.44 |
| ATOM | 2614 | OG1 | THR | B | 537 | 24.336 | 67.782 | 12.890 | 1.00 | 47.61 |
| ATOM | 2615 | CG2 | THR | B | 537 | 25.471 | 66.356 | 11.307 | 1.00 | 47.41 |
| ATOM | 2616 | C | THR | B | 537 | 24.866 | 65.684 | 15.026 | 1.00 | 50.26 |
| ATOM | 2617 | O | THR | B | 537 | 24.138 | 65.096 | 15.828 | 1.00 | 50.63 |
| ATOM | 2618 | N | THR | B | 537 | 23.266 | 65.220 | 13.181 | 1.00 | 50.23 |
| ATOM | 2619 | CA | THR | B | 537 | 24.695 | 65.462 | 13.530 | 1.00 | 50.15 |
| ATOM | 2620 | N | ARG | B | 538 | 25.822 | 66.534 | 15.395 | 1.00 | 48.27 |
| ATOM | 2621 | CA | ARG | B | 538 | 26.099 | 66.827 | 16.801 | 1.00 | 48.37 |
| ATOM | 2622 | CB | ARG | B | 538 | 24.804 | 67.211 | 17.532 | 1.00 | 55.34 |
| ATOM | 2623 | CG | ARG | B | 538 | 24.941 | 67.437 | 19.040 | 1.00 | 55.68 |
| ATOM | 2624 | CD | ARG | B | 538 | 25.783 | 68.664 | 19.378 | 1.00 | 55.76 |
| ATOM | 2625 | NE | ARG | B | 538 | 25.806 | 68.927 | 20.818 | 1.00 | 55.63 |
| ATOM | 2626 | CZ | ARG | B | 538 | 26.529 | 69.882 | 21.401 | 1.00 | 55.83 |
| ATOM | 2627 | NH1 | ARG | B | 538 | 27.302 | 70.677 | 20.672 | 1.00 | 55.58 |
| ATOM | 2628 | NH2 | ARG | B | 538 | 26.481 | 70.040 | 22.718 | 1.00 | 55.68 |
| ATOM | 2629 | C | ARG | B | 538 | 26.707 | 65.579 | 17.427 | 1.00 | 48.47 |
| ATOM | 2630 | O | ARG | B | 538 | 27.882 | 65.561 | 17.796 | 1.00 | 47.96 |
| ATOM | 2631 | N | GLU | B | 539 | 25.895 | 64.534 | 17.532 | 1.00 | 51.41 |
| ATOM | 2632 | CA | GLU | B | 539 | 26.331 | 63.265 | 18.096 | 1.00 | 50.79 |
| ATOM | 2633 | CB | GLU | B | 539 | 25.162 | 62.280 | 18.093 | 1.00 | 45.27 |
| ATOM | 2634 | CG | GLU | B | 539 | 25.499 | 60.877 | 18.559 | 1.00 | 45.43 |
| ATOM | 2635 | CD | GLU | B | 539 | 24.274 | 59.985 | 18.595 | 1.00 | 45.09 |
| ATOM | 2636 | OE1 | GLU | B | 539 | 23.553 | 59.931 | 17.576 | 1.00 | 45.02 |
| ATOM | 2637 | OE2 | GLU | B | 539 | 24.032 | 59.340 | 19.636 | 1.00 | 45.34 |
| ATOM | 2638 | C | GLU | B | 539 | 27.490 | 62.719 | 17.268 | 1.00 | 49.89 |
| ATOM | 2639 | O | GLU | B | 539 | 28.360 | 62.015 | 17.783 | 1.00 | 51.14 |
| ATOM | 2640 | N | LEU | B | 540 | 27.493 | 63.056 | 15.981 | 1.00 | 35.39 |
| ATOM | 2641 | CA | LEU | B | 540 | 28.537 | 62.617 | 15.066 | 1.00 | 34.61 |
| ATOM | 2642 | CB | LEU | B | 540 | 28.047 | 62.729 | 13.621 | 1.00 | 33.85 |
| ATOM | 2643 | CG | LEU | B | 540 | 29.094 | 62.491 | 12.529 | 1.00 | 33.88 |
| ATOM | 2644 | CD1 | LEU | B | 540 | 29.755 | 61.135 | 12.722 | 1.00 | 33.93 |
| ATOM | 2645 | CD2 | LEU | B | 540 | 28.436 | 62.579 | 11.166 | 1.00 | 34.03 |
| ATOM | 2646 | C | LEU | B | 540 | 29.798 | 63.451 | 15.245 | 1.00 | 33.92 |
| ATOM | 2647 | O | LEU | B | 540 | 30.910 | 62.923 | 15.296 | 1.00 | 33.74 |
| ATOM | 2648 | N | GLN | B | 541 | 29.620 | 64.763 | 15.328 | 1.00 | 38.91 |
| ATOM | 2649 | CA | GLN | B | 541 | 30.747 | 65.664 | 15.511 | 1.00 | 38.96 |
| ATOM | 2650 | CB | GLN | B | 541 | 30.265 | 67.110 | 15.475 | 1.00 | 49.53 |
| ATOM | 2651 | CG | GLN | B | 541 | 29.654 | 67.501 | 14.147 | 1.00 | 49.81 |
| ATOM | 2652 | CD | GLN | B | 541 | 29.118 | 68.910 | 14.156 | 1.00 | 49.94 |
| ATOM | 2653 | OE1 | GLN | B | 541 | 28.185 | 69.225 | 14.896 | 1.00 | 50.34 |
| ATOM | 2654 | NE2 | GLN | B | 541 | 29.707 | 69.774 | 13.336 | 1.00 | 50.07 |
| ATOM | 2655 | C | GLN | B | 541 | 31.389 | 65.356 | 16.850 | 1.00 | 38.55 |
| ATOM | 2656 | O | GLN | B | 541 | 32.613 | 65.343 | 16.980 | 1.00 | 39.00 |
| ATOM | 2657 | N | SER | B | 542 | 30.551 | 65.099 | 17.847 | 1.00 | 32.80 |
| ATOM | 2658 | CA | SER | B | 542 | 31.034 | 64.772 | 19.177 | 1.00 | 32.42 |
| ATOM | 2659 | CB | SER | B | 542 | 29.861 | 64.645 | 20.153 | 1.00 | 41.30 |
| ATOM | 2660 | OG | SER | B | 542 | 29.144 | 65.863 | 20.263 | 1.00 | 42.04 |
| ATOM | 2661 | C | SER | B | 542 | 31.789 | 63.456 | 19.113 | 1.00 | 30.70 |
| ATOM | 2662 | O | SER | B | 542 | 32.848 | 63.305 | 19.714 | 1.00 | 31.49 |
| ATOM | 2663 | N | LEU | B | 543 | 31.233 | 62.505 | 18.370 | 1.00 | 31.05 |
| ATOM | 2664 | CA | LEU | B | 543 | 31.841 | 61.188 | 18.228 | 1.00 | 30.13 |
| ATOM | 2665 | CB | LEU | B | 543 | 30.895 | 60.257 | 17.460 | 1.00 | 31.49 |
| ATOM | 2666 | CG | LEU | B | 543 | 30.924 | 58.757 | 17.783 | 1.00 | 32.02 |
| ATOM | 2667 | CD1 | LEU | B | 543 | 30.315 | 57.991 | 16.615 | 1.00 | 31.51 |
| ATOM | 2668 | CD2 | LEU | B | 543 | 32.336 | 58.278 | 18.036 | 1.00 | 31.28 |
| ATOM | 2669 | C | LEU | B | 543 | 33.175 | 61.259 | 17.492 | 1.00 | 29.52 |
| ATOM | 2670 | O | LEU | B | 543 | 34.183 | 60.737 | 17.963 | 1.00 | 29.71 |
| ATOM | 2671 | N | ALA | B | 544 | 33.175 | 61.911 | 16.335 | 1.00 | 27.41 |
| ATOM | 2672 | CA | ALA | B | 544 | 34.374 | 62.027 | 15.522 | 1.00 | 26.77 |
| ATOM | 2673 | CB | ALA | B | 544 | 34.040 | 62.722 | 14.213 | 1.00 | 32.00 |
| ATOM | 2674 | C | ALA | B | 544 | 35.529 | 62.748 | 16.214 | 1.00 | 27.56 |
| ATOM | 2675 | O | ALA | B | 544 | 36.685 | 62.357 | 16.075 | 1.00 | 27.33 |
| ATOM | 2676 | N | ALA | B | 545 | 35.214 | 63.801 | 16.960 | 1.00 | 29.78 |
| ATOM | 2677 | CA | ALA | B | 545 | 36.241 | 64.580 | 17.642 | 1.00 | 29.66 |
| ATOM | 2678 | CB | ALA | B | 545 | 35.723 | 65.990 | 17.905 | 1.00 | 33.66 |
| ATOM | 2679 | C | ALA | B | 545 | 36.731 | 63.962 | 18.948 | 1.00 | 30.65 |
| ATOM | 2680 | O | ALA | B | 545 | 37.829 | 64.272 | 19.417 | 1.00 | 29.67 |
| ATOM | 2681 | N | ALA | B | 546 | 35.921 | 63.090 | 19.537 | 1.00 | 29.20 |
| ATOM | 2682 | CA | ALA | B | 546 | 36.284 | 62.464 | 20.801 | 1.00 | 30.23 |
| ATOM | 2683 | CB | ALA | B | 546 | 35.090 | 61.705 | 21.364 | 1.00 | 34.60 |
| ATOM | 2684 | C | ALA | B | 546 | 37.493 | 61.538 | 20.713 | 1.00 | 29.49 |
| ATOM | 2685 | O | ALA | B | 546 | 37.779 | 60.959 | 19.664 | 1.00 | 29.95 |
| ATOM | 2686 | N | VAL | B | 547 | 38.219 | 61.423 | 21.820 | 1.00 | 29.24 |
| ATOM | 2687 | CA | VAL | B | 547 | 39.377 | 60.543 | 21.872 | 1.00 | 29.27 |
| ATOM | 2688 | CB | VAL | B | 547 | 40.359 | 60.950 | 22.990 | 1.00 | 28.05 |
| ATOM | 2689 | CG1 | VAL | B | 547 | 41.419 | 59.877 | 23.158 | 1.00 | 27.67 |
| ATOM | 2690 | CG2 | VAL | B | 547 | 41.010 | 62.282 | 22.650 | 1.00 | 28.10 |
| ATOM | 2691 | C | VAL | B | 547 | 38.845 | 59.151 | 22.179 | 1.00 | 29.34 |
| ATOM | 2692 | O | VAL | B | 547 | 38.009 | 58.988 | 23.066 | 1.00 | 30.26 |
| ATOM | 2693 | N | VAL | B | 548 | 39.324 | 58.152 | 21.443 | 1.00 | 28.29 |
| ATOM | 2694 | CA | VAL | B | 548 | 38.879 | 56.778 | 21.653 | 1.00 | 27.44 |
| ATOM | 2695 | CB | VAL | B | 548 | 38.971 | 55.953 | 20.349 | 1.00 | 28.22 |
| ATOM | 2696 | CG1 | VAL | B | 548 | 38.545 | 54.510 | 20.613 | 1.00 | 27.84 |
| ATOM | 2697 | CG2 | VAL | B | 548 | 38.100 | 56.585 | 19.271 | 1.00 | 28.24 |
| ATOM | 2698 | C | VAL | B | 548 | 39.721 | 56.099 | 22.724 | 1.00 | 26.75 |
| ATOM | 2699 | O | VAL | B | 548 | 40.903 | 55.822 | 22.518 | 1.00 | 26.81 |
| ATOM | 2700 | N | PRO | B | 549 | 39.121 | 55.826 | 23.890 | 1.00 | 24.59 |
| ATOM | 2701 | CD | PRO | B | 549 | 37.783 | 56.267 | 24.317 | 1.00 | 25.72 |
| ATOM | 2702 | CA | PRO | B | 549 | 39.830 | 55.172 | 24.995 | 1.00 | 25.08 |
| ATOM | 2703 | CB | PRO | B | 549 | 38.764 | 55.089 | 26.081 | 1.00 | 25.73 |
| ATOM | 2704 | CG | PRO | B | 549 | 37.932 | 56.312 | 25.818 | 1.00 | 26.01 |
| ATOM | 2705 | C | PRO | B | 549 | 40.362 | 53.795 | 24.600 | 1.00 | 26.03 |
| ATOM | 2706 | O | PRO | B | 549 | 39.894 | 53.197 | 23.628 | 1.00 | 24.91 |
| ATOM | 2707 | N | SER | B | 550 | 41.334 | 53.298 | 25.361 | 1.00 | 27.34 |
| ATOM | 2708 | CA | SER | B | 550 | 41.935 | 51.997 | 25.088 | 1.00 | 27.02 |
| ATOM | 2709 | CB | SER | B | 550 | 43.224 | 51.821 | 25.896 | 1.00 | 31.88 |
| ATOM | 2710 | OG | SER | B | 550 | 42.943 | 51.600 | 27.267 | 1.00 | 31.82 |
| ATOM | 2711 | C | SER | B | 550 | 40.975 | 50.873 | 25.440 | 1.00 | 26.64 |
| ATOM | 2712 | O | SER | B | 550 | 40.052 | 51.062 | 26.229 | 1.00 | 26.84 |
| ATOM | 2713 | N | ALA | B | 551 | 41.193 | 49.701 | 24.853 | 1.00 | 26.78 |
| ATOM | 2714 | CA | ALA | B | 551 | 40.336 | 48.558 | 25.135 | 1.00 | 27.11 |
| ATOM | 2715 | CB | ALA | B | 551 | 40.790 | 47.342 | 24.317 | 1.00 | 24.60 |
| ATOM | 2716 | C | ALA | B | 551 | 40.385 | 48.244 | 26.630 | 1.00 | 27.04 |
| ATOM | 2717 | O | ALA | B | 551 | 39.373 | 47.895 | 27.237 | 1.00 | 26.84 |
| ATOM | 2718 | N | GLN | B | 552 | 41.573 | 48.370 | 27.213 | 1.00 | 28.38 |
| ATOM | 2719 | CA | GLN | B | 552 | 41.775 | 48.112 | 28.636 | 1.00 | 29.31 |
| ATOM | 2720 | CB | GLN | B | 552 | 43.233 | 48.374 | 29.014 | 1.00 | 41.60 |
| ATOM | 2721 | CG | GLN | B | 552 | 44.219 | 47.373 | 28.448 | 1.00 | 42.50 |
| ATOM | 2722 | CD | GLN | B | 552 | 45.646 | 47.898 | 28.453 | 1.00 | 42.52 |
| ATOM | 2723 | OE1 | GLN | B | 552 | 46.015 | 48.738 | 27.626 | 1.00 | 42.19 |
| ATOM | 2724 | NE2 | GLN | B | 552 | 46.453 | 47.413 | 29.392 | 1.00 | 42.63 |
| ATOM | 2725 | C | GLN | B | 552 | 40.878 | 49.011 | 29.475 | 1.00 | 28.87 |
| ATOM | 2726 | O | GLN | B | 552 | 40.149 | 48.544 | 30.355 | 1.00 | 28.59 |
| ATOM | 2727 | N | THR | B | 553 | 40.944 | 50.307 | 29.196 | 1.00 | 30.11 |
| ATOM | 2728 | CA | THR | B | 553 | 40.145 | 51.283 | 29.915 | 1.00 | 29.74 |
| ATOM | 2729 | CB | THR | B | 553 | 40.415 | 52.713 | 29.399 | 1.00 | 32.61 |
| ATOM | 2730 | OG1 | THR | B | 553 | 41.795 | 53.047 | 29.598 | 1.00 | 32.65 |
| ATOM | 2731 | CG2 | THR | B | 553 | 39.541 | 53.718 | 30.142 | 1.00 | 32.91 |
| ATOM | 2732 | C | THR | B | 553 | 38.655 | 50.990 | 29.765 | 1.00 | 29.20 |
| ATOM | 2733 | O | THR | B | 553 | 37.919 | 50.996 | 30.747 | 1.00 | 29.52 |
| ATOM | 2734 | N | LEU | B | 554 | 38.225 | 50.723 | 28.534 | 1.00 | 27.01 |
| ATOM | 2735 | CA | LEU | B | 554 | 36.820 | 50.455 | 28.244 | 1.00 | 26.50 |
| ATOM | 2736 | CB | LEU | B | 554 | 36.562 | 50.590 | 26.741 | 1.00 | 28.84 |
| ATOM | 2737 | CG | LEU | B | 554 | 36.851 | 51.973 | 26.146 | 1.00 | 29.16 |
| ATOM | 2738 | CD1 | LEU | B | 554 | 36.766 | 51.908 | 24.630 | 1.00 | 29.33 |
| ATOM | 2739 | CD2 | LEU | B | 554 | 35.862 | 52.992 | 26.699 | 1.00 | 29.62 |
| ATOM | 2740 | C | LEU | B | 554 | 36.324 | 49.098 | 28.733 | 1.00 | 26.37 |
| ATOM | 2741 | O | LEU | B | 554 | 35.117 | 48.879 | 28.832 | 1.00 | 26.37 |
| ATOM | 2742 | N | LYS | B | 555 | 37.255 | 48.192 | 29.026 | 1.00 | 25.28 |
| ATOM | 2743 | CA | LYS | B | 555 | 36.931 | 46.857 | 29.524 | 1.00 | 24.39 |
| ATOM | 2744 | CB | LYS | B | 555 | 36.089 | 46.958 | 30.802 | 1.00 | 35.97 |
| ATOM | 2745 | CG | LYS | B | 555 | 36.740 | 47.722 | 31.945 | 1.00 | 36.48 |
| ATOM | 2746 | CD | LYS | B | 555 | 35.806 | 47.781 | 33.150 | 1.00 | 36.95 |
| ATOM | 2747 | CE | LYS | B | 555 | 36.409 | 48.579 | 34.297 | 1.00 | 36.81 |
| ATOM | 2748 | NZ | LYS | B | 555 | 35.499 | 48.633 | 35.478 | 1.00 | 37.17 |
| ATOM | 2749 | C | LYS | B | 555 | 36.188 | 45.975 | 28.520 | 1.00 | 24.71 |
| ATOM | 2750 | O | LYS | B | 555 | 35.592 | 44.966 | 28.905 | 1.00 | 24.80 |
| ATOM | 2751 | N | ILE | B | 556 | 36.229 | 46.336 | 27.242 | 1.00 | 24.43 |
| ATOM | 2752 | CA | ILE | B | 556 | 35.520 | 45.556 | 26.231 | 1.00 | 24.37 |
| ATOM | 2753 | CB | ILE | B | 556 | 35.436 | 46.312 | 24.895 | 1.00 | 23.67 |
| ATOM | 2754 | CG2 | ILE | B | 556 | 34.651 | 47.601 | 25.078 | 1.00 | 24.41 |
| ATOM | 2755 | CG1 | ILE | B | 556 | 36.838 | 46.637 | 24.384 | 1.00 | 24.28 |
| ATOM | 2756 | CD1 | ILE | B | 556 | 36.847 | 47.138 | 22.956 | 1.00 | 23.53 |
| ATOM | 2757 | C | ILE | B | 556 | 36.095 | 44.167 | 25.966 | 1.00 | 23.99 |
| ATOM | 2758 | O | ILE | B | 556 | 35.499 | 43.380 | 25.235 | 1.00 | 23.82 |
| ATOM | 2759 | N | THR | B | 557 | 37.246 | 43.860 | 26.551 | 1.00 | 24.82 |
| ATOM | 2760 | CA | THR | B | 557 | 37.843 | 42.545 | 26.345 | 1.00 | 25.70 |
| ATOM | 2761 | CB | THR | B | 557 | 39.381 | 42.593 | 26.486 | 1.00 | 34.81 |
| ATOM | 2762 | OG1 | THR | B | 557 | 39.901 | 43.678 | 25.706 | 1.00 | 34.99 |
| ATOM | 2763 | CG2 | THR | B | 557 | 40.001 | 41.289 | 25.991 | 1.00 | 34.84 |
| ATOM | 2764 | C | THR | B | 557 | 37.289 | 41.526 | 27.344 | 1.00 | 25.75 |
| ATOM | 2765 | O | THR | B | 557 | 37.425 | 40.318 | 27.154 | 1.00 | 25.95 |
| ATOM | 2766 | N | ASP | B | 558 | 36.646 | 42.013 | 28.400 | 1.00 | 21.17 |
| ATOM | 2767 | CA | ASP | B | 558 | 36.099 | 41.126 | 29.423 | 1.00 | 20.89 |
| ATOM | 2768 | CB | ASP | B | 558 | 35.952 | 41.873 | 30.750 | 1.00 | 39.26 |
| ATOM | 2769 | CG | ASP | B | 558 | 37.214 | 42.607 | 31.147 | 1.00 | 40.04 |
| ATOM | 2770 | OD1 | ASP | B | 558 | 38.293 | 41.981 | 31.148 | 1.00 | 40.44 |
| ATOM | 2771 | OD2 | ASP | B | 558 | 37.124 | 43.809 | 31.464 | 1.00 | 40.62 |
| ATOM | 2772 | C | ASP | B | 558 | 34.742 | 40.543 | 29.045 | 1.00 | 21.50 |
| ATOM | 2773 | O | ASP | B | 558 | 33.846 | 41.259 | 28.594 | 1.00 | 21.10 |
| ATOM | 2774 | N | PHE | B | 559 | 34.586 | 39.242 | 29.246 | 1.00 | 27.65 |
| ATOM | 2775 | CA | PHE | B | 559 | 33.322 | 38.583 | 28.942 | 1.00 | 28.56 |
| ATOM | 2776 | CB | PHE | B | 559 | 33.474 | 37.067 | 29.076 | 1.00 | 27.72 |
| ATOM | 2777 | CG | PHE | B | 559 | 34.269 | 36.438 | 27.968 | 1.00 | 27.53 |
| ATOM | 2778 | CD1 | PHE | B | 559 | 33.773 | 36.407 | 26.670 | 1.00 | 27.89 |
| ATOM | 2779 | CD2 | PHE | B | 559 | 35.509 | 35.863 | 28.224 | 1.00 | 27.40 |
| ATOM | 2780 | CE1 | PHE | B | 559 | 34.499 | 35.809 | 25.642 | 1.00 | 27.88 |
| ATOM | 2781 | CE2 | PHE | B | 559 | 36.243 | 35.263 | 27.202 | 1.00 | 27.42 |
| ATOM | 2782 | CZ | PHE | B | 559 | 35.738 | 35.236 | 25.911 | 1.00 | 27.16 |
| ATOM | 2783 | C | PHE | B | 559 | 32.214 | 39.072 | 29.872 | 1.00 | 28.90 |
| ATOM | 2784 | O | PHE | B | 559 | 31.030 | 38.983 | 29.545 | 1.00 | 28.49 |
| ATOM | 2785 | N | SER | B | 560 | 32.604 | 39.597 | 31.029 | 1.00 | 29.75 |
| ATOM | 2786 | CA | SER | B | 560 | 31.638 | 40.087 | 32.008 | 1.00 | 30.25 |
| ATOM | 2787 | CB | SER | B | 560 | 32.204 | 39.924 | 33.421 | 1.00 | 41.19 |
| ATOM | 2788 | OG | SER | B | 560 | 33.408 | 40.654 | 33.576 | 1.00 | 41.28 |
| ATOM | 2789 | C | SER | B | 560 | 31.248 | 41.543 | 31.778 | 1.00 | 30.07 |
| ATOM | 2790 | O | SER | B | 560 | 30.571 | 42.149 | 32.609 | 1.00 | 29.90 |
| ATOM | 2791 | N | PHE | B | 561 | 31.674 | 42.098 | 30.648 | 1.00 | 25.27 |
| ATOM | 2792 | CA | PHE | B | 561 | 31.377 | 43.484 | 30.297 | 1.00 | 24.91 |
| ATOM | 2793 | CB | PHE | B | 561 | 31.775 | 43.744 | 28.842 | 1.00 | 26.48 |
| ATOM | 2794 | CG | PHE | B | 561 | 31.458 | 45.135 | 28.357 | 1.00 | 26.42 |
| ATOM | 2795 | CD1 | PHE | B | 561 | 32.349 | 46.181 | 28.564 | 1.00 | 26.47 |
| ATOM | 2796 | CD2 | PHE | B | 561 | 30.267 | 45.392 | 27.679 | 1.00 | 26.27 |
| ATOM | 2797 | CE1 | PHE | B | 561 | 32.061 | 47.468 | 28.100 | 1.00 | 27.33 |
| ATOM | 2798 | CE2 | PHE | B | 561 | 29.970 | 46.676 | 27.210 | 1.00 | 26.55 |
| ATOM | 2799 | CZ | PHE | B | 561 | 30.870 | 47.715 | 27.421 | 1.00 | 26.39 |
| ATOM | 2800 | C | PHE | B | 561 | 29.904 | 43.837 | 30.476 | 1.00 | 24.69 |
| ATOM | 2801 | O | PHE | B | 561 | 29.020 | 43.040 | 30.170 | 1.00 | 25.07 |
| ATOM | 2802 | N | SER | B | 562 | 29.652 | 45.047 | 30.966 | 1.00 | 26.30 |
| ATOM | 2803 | CA | SER | B | 562 | 28.291 | 45.538 | 31.163 | 1.00 | 27.46 |
| ATOM | 2804 | CB | SER | B | 562 | 27.992 | 45.694 | 32.655 | 1.00 | 44.74 |
| ATOM | 2805 | OG | SER | B | 562 | 26.657 | 46.118 | 32.861 | 1.00 | 45.35 |
| ATOM | 2806 | C | SER | B | 562 | 28.188 | 46.895 | 30.476 | 1.00 | 25.80 |
| ATOM | 2807 | O | SER | B | 562 | 29.092 | 47.721 | 30.593 | 1.00 | 25.80 |
| ATOM | 2808 | N | ASP | B | 563 | 27.094 | 47.120 | 29.756 | 1.00 | 22.70 |
| ATOM | 2809 | CA | ASP | B | 563 | 26.896 | 48.380 | 29.050 | 1.00 | 23.18 |
| ATOM | 2810 | CB | ASP | B | 563 | 26.203 | 48.138 | 27.709 | 1.00 | 23.75 |
| ATOM | 2811 | CG | ASP | B | 563 | 24.700 | 47.931 | 27.861 | 1.00 | 23.45 |
| ATOM | 2812 | OD1 | ASP | B | 563 | 24.282 | 46.853 | 28.330 | 1.00 | 23.97 |
| ATOM | 2813 | OD2 | ASP | B | 563 | 23.936 | 48.858 | 27.522 | 1.00 | 24.26 |
| ATOM | 2814 | C | ASP | B | 563 | 26.014 | 49.330 | 29.847 | 1.00 | 22.69 |
| ATOM | 2815 | O | ASP | B | 563 | 25.889 | 50.504 | 29.503 | 1.00 | 23.59 |
| ATOM | 2816 | N | PHE | B | 564 | 25.398 | 48.813 | 30.902 | 1.00 | 25.43 |
| ATOM | 2817 | CA | PHE | B | 564 | 24.471 | 49.599 | 31.708 | 1.00 | 26.75 |
| ATOM | 2818 | CB | PHE | B | 564 | 24.056 | 48.800 | 32.942 | 1.00 | 37.63 |
| ATOM | 2819 | CG | PHE | B | 564 | 22.817 | 49.324 | 33.606 | 1.00 | 38.09 |
| ATOM | 2820 | CD1 | PHE | B | 564 | 21.604 | 49.337 | 32.921 | 1.00 | 38.40 |
| ATOM | 2821 | CD2 | PHE | B | 564 | 22.858 | 49.809 | 34.907 | 1.00 | 38.36 |
| ATOM | 2822 | CE1 | PHE | B | 564 | 20.446 | 49.826 | 33.525 | 1.00 | 38.54 |
| ATOM | 2823 | CE2 | PHE | B | 564 | 21.706 | 50.301 | 35.522 | 1.00 | 38.90 |
| ATOM | 2824 | CZ | PHE | B | 564 | 20.498 | 50.309 | 34.829 | 1.00 | 38.44 |
| ATOM | 2825 | C | PHE | B | 564 | 24.907 | 50.999 | 32.137 | 1.00 | 27.15 |
| ATOM | 2826 | O | PHE | B | 564 | 24.118 | 51.945 | 32.064 | 1.00 | 26.92 |
| ATOM | 2827 | N | GLU | B | 565 | 26.156 | 51.139 | 32.566 | 1.00 | 29.91 |
| ATOM | 2828 | CA | GLU | B | 565 | 26.654 | 52.431 | 33.034 | 1.00 | 30.93 |
| ATOM | 2829 | CB | GLU | B | 565 | 27.762 | 52.219 | 34.069 | 1.00 | 40.08 |
| ATOM | 2830 | CG | GLU | B | 565 | 27.372 | 51.367 | 35.270 | 1.00 | 40.03 |
| ATOM | 2831 | CD | GLU | B | 565 | 27.050 | 49.935 | 34.890 | 1.00 | 40.48 |
| ATOM | 2832 | OE1 | GLU | B | 565 | 27.787 | 49.363 | 34.058 | 1.00 | 40.51 |
| ATOM | 2833 | OE2 | GLU | B | 565 | 26.066 | 49.378 | 35.428 | 1.00 | 40.01 |
| ATOM | 2834 | C | GLU | B | 565 | 27.173 | 53.361 | 31.944 | 1.00 | 30.09 |
| ATOM | 2835 | O | GLU | B | 565 | 27.567 | 54.497 | 32.227 | 1.00 | 30.95 |
| ATOM | 2836 | N | LEU | B | 566 | 27.167 | 52.894 | 30.700 | 1.00 | 27.08 |
| ATOM | 2837 | CA | LEU | B | 566 | 27.671 | 53.692 | 29.594 | 1.00 | 26.42 |
| ATOM | 2838 | CB | LEU | B | 566 | 28.395 | 52.784 | 28.591 | 1.00 | 35.46 |
| ATOM | 2839 | CG | LEU | B | 566 | 29.522 | 51.884 | 29.106 | 1.00 | 36.46 |
| ATOM | 2840 | CD1 | LEU | B | 566 | 30.022 | 50.996 | 27.969 | 1.00 | 35.95 |
| ATOM | 2841 | CD2 | LEU | B | 566 | 30.659 | 52.730 | 29.659 | 1.00 | 36.32 |
| ATOM | 2842 | C | LEU | B | 566 | 26.600 | 54.483 | 28.860 | 1.00 | 25.95 |
| ATOM | 2843 | O | LEU | B | 566 | 25.454 | 54.048 | 28.756 | 1.00 | 24.61 |
| ATOM | 2844 | N | SER | B | 567 | 26.986 | 55.655 | 28.358 | 1.00 | 25.45 |
| ATOM | 2845 | CA | SER | B | 567 | 26.080 | 56.501 | 27.590 | 1.00 | 25.56 |
| ATOM | 2846 | CB | SER | B | 567 | 26.519 | 57.963 | 27.665 | 1.00 | 33.98 |
| ATOM | 2847 | OG | SER | B | 567 | 27.722 | 58.173 | 26.947 | 1.00 | 34.14 |
| ATOM | 2848 | C | SER | B | 567 | 26.155 | 56.027 | 26.140 | 1.00 | 25.50 |
| ATOM | 2849 | O | SER | B | 567 | 27.047 | 55.247 | 25.791 | 1.00 | 23.97 |
| ATOM | 2850 | N | ASP | B | 568 | 25.234 | 56.490 | 25.300 | 1.00 | 26.74 |
| ATOM | 2851 | CA | ASP | B | 568 | 25.240 | 56.101 | 23.892 | 1.00 | 26.93 |
| ATOM | 2852 | CB | ASP | B | 568 | 24.110 | 56.790 | 23.127 | 1.00 | 31.14 |
| ATOM | 2853 | CG | ASP | B | 568 | 22.760 | 56.161 | 23.390 | 1.00 | 31.71 |
| ATOM | 2854 | OD1 | ASP | B | 568 | 21.783 | 56.584 | 22.743 | 1.00 | 32.07 |
| ATOM | 2855 | OD2 | ASP | B | 568 | 22.678 | 55.246 | 24.240 | 1.00 | 31.51 |
| ATOM | 2856 | C | ASP | B | 568 | 26.569 | 56.455 | 23.241 | 1.00 | 26.60 |
| ATOM | 2857 | O | ASP | B | 568 | 27.130 | 55.656 | 22.494 | 1.00 | 26.36 |
| ATOM | 2858 | N | LEU | B | 569 | 27.071 | 57.656 | 23.526 | 1.00 | 26.00 |
| ATOM | 2859 | CA | LEU | B | 569 | 28.341 | 58.091 | 22.964 | 1.00 | 25.02 |
| ATOM | 2860 | CB | LEU | B | 569 | 28.683 | 59.507 | 23.446 | 1.00 | 35.36 |
| ATOM | 2861 | CG | LEU | B | 569 | 30.009 | 60.124 | 22.980 | 1.00 | 35.65 |
| ATOM | 2862 | CD1 | LEU | B | 569 | 30.050 | 60.203 | 21.464 | 1.00 | 36.29 |
| ATOM | 2863 | CD2 | LEU | B | 569 | 30.153 | 61.516 | 23.583 | 1.00 | 35.93 |
| ATOM | 2864 | C | LEU | B | 569 | 29.432 | 57.113 | 23.388 | 1.00 | 24.35 |
| ATOM | 2865 | O | LEU | B | 569 | 30.267 | 56.716 | 22.574 | 1.00 | 25.31 |
| ATOM | 2866 | N | GLU | B | 570 | 29.412 | 56.715 | 24.659 | 1.00 | 23.22 |
| ATOM | 2867 | CA | GLU | B | 570 | 30.406 | 55.784 | 25.173 | 1.00 | 23.38 |
| ATOM | 2868 | CB | GLU | B | 570 | 30.216 | 55.560 | 26.676 | 1.00 | 33.69 |
| ATOM | 2869 | CG | GLU | B | 570 | 30.567 | 56.787 | 27.522 | 1.00 | 34.19 |
| ATOM | 2870 | CD | GLU | B | 570 | 30.543 | 56.509 | 29.017 | 1.00 | 34.09 |
| ATOM | 2871 | OE1 | GLU | B | 570 | 29.467 | 56.160 | 29.548 | 1.00 | 33.38 |
| ATOM | 2872 | OE2 | GLU | B | 570 | 31.605 | 56.642 | 29.665 | 1.00 | 35.27 |
| ATOM | 2873 | C | GLU | B | 570 | 30.351 | 54.458 | 24.423 | 1.00 | 23.12 |
| ATOM | 2874 | O | GLU | B | 570 | 31.397 | 53.899 | 24.085 | 1.00 | 23.04 |
| ATOM | 2875 | N | THR | B | 571 | 29.148 | 53.954 | 24.155 | 1.00 | 23.20 |
| ATOM | 2876 | CA | THR | B | 571 | 29.038 | 52.692 | 23.423 | 1.00 | 23.72 |
| ATOM | 2877 | CB | THR | B | 571 | 27.582 | 52.146 | 23.367 | 1.00 | 22.43 |
| ATOM | 2878 | OG1 | THR | B | 571 | 26.753 | 53.014 | 22.589 | 1.00 | 23.16 |
| ATOM | 2879 | CG2 | THR | B | 571 | 27.010 | 52.013 | 24.772 | 1.00 | 23.40 |
| ATOM | 2880 | C | THR | B | 571 | 29.564 | 52.865 | 22.000 | 1.00 | 22.59 |
| ATOM | 2881 | O | THR | B | 571 | 30.147 | 51.937 | 21.441 | 1.00 | 23.35 |
| ATOM | 2882 | N | ALA | B | 572 | 29.367 | 54.047 | 21.418 | 1.00 | 23.15 |
| ATOM | 2883 | CA | ALA | B | 572 | 29.852 | 54.323 | 20.065 | 1.00 | 23.02 |
| ATOM | 2884 | CB | ALA | B | 572 | 29.330 | 55.671 | 19.575 | 1.00 | 25.21 |
| ATOM | 2885 | C | ALA | B | 572 | 31.381 | 54.315 | 20.061 | 1.00 | 24.49 |
| ATOM | 2886 | O | ALA | B | 572 | 32.007 | 53.797 | 19.133 | 1.00 | 23.72 |
| ATOM | 2887 | N | LEU | B | 573 | 31.984 | 54.906 | 21.092 | 1.00 | 24.96 |
| ATOM | 2888 | CA | LEU | B | 573 | 33.438 | 54.924 | 21.203 | 1.00 | 25.56 |
| ATOM | 2889 | CB | LEU | B | 573 | 33.870 | 55.787 | 22.395 | 1.00 | 25.61 |
| ATOM | 2890 | CG | LEU | B | 573 | 33.684 | 57.294 | 22.204 | 1.00 | 25.96 |
| ATOM | 2891 | CD1 | LEU | B | 573 | 33.998 | 58.025 | 23.505 | 1.00 | 25.46 |
| ATOM | 2892 | CD2 | LEU | B | 573 | 34.581 | 57.784 | 21.076 | 1.00 | 24.77 |
| ATOM | 2893 | C | LEU | B | 573 | 33.961 | 53.490 | 21.363 | 1.00 | 23.87 |
| ATOM | 2894 | O | LEU | B | 573 | 34.960 | 53.116 | 20.744 | 1.00 | 25.16 |
| ATOM | 2895 | N | CYS | B | 574 | 33.291 | 52.697 | 22.195 | 1.00 | 21.17 |
| ATOM | 2896 | CA | CYS | B | 574 | 33.672 | 51.297 | 22.406 | 1.00 | 20.66 |
| ATOM | 2897 | CB | CYS | B | 574 | 32.687 | 50.593 | 23.342 | 1.00 | 22.74 |
| ATOM | 2898 | SG | CYS | B | 574 | 32.882 | 50.908 | 25.120 | 1.00 | 21.69 |
| ATOM | 2899 | C | CYS | B | 574 | 33.665 | 50.555 | 21.072 | 1.00 | 21.23 |
| ATOM | 2900 | O | CYS | B | 574 | 34.536 | 49.727 | 20.801 | 1.00 | 19.77 |
| ATOM | 2901 | N | THR | B | 575 | 32.668 | 50.849 | 20.248 | 1.00 | 21.05 |
| ATOM | 2902 | CA | THR | B | 575 | 32.543 | 50.203 | 18.948 | 1.00 | 20.83 |
| ATOM | 2903 | CB | THR | B | 575 | 31.211 | 50.590 | 18.273 | 1.00 | 21.54 |
| ATOM | 2904 | OG1 | THR | B | 575 | 30.128 | 50.166 | 19.109 | 1.00 | 22.40 |
| ATOM | 2905 | CG2 | THR | B | 575 | 31.067 | 49.917 | 16.912 | 1.00 | 22.61 |
| ATOM | 2906 | C | THR | B | 575 | 33.722 | 50.565 | 18.052 | 1.00 | 21.63 |
| ATOM | 2907 | O | THR | B | 575 | 34.268 | 49.708 | 17.357 | 1.00 | 20.92 |
| ATOM | 2908 | N | ILE | B | 576 | 34.122 | 51.833 | 18.068 | 1.00 | 21.26 |
| ATOM | 2909 | CA | ILE | B | 576 | 35.256 | 52.261 | 17.254 | 1.00 | 21.61 |
| ATOM | 2910 | CB | ILE | B | 576 | 35.510 | 53.786 | 17.365 | 1.00 | 24.97 |
| ATOM | 2911 | CG2 | ILE | B | 576 | 36.817 | 54.147 | 16.660 | 1.00 | 25.32 |
| ATOM | 2912 | CG1 | ILE | B | 576 | 34.333 | 54.558 | 16.757 | 1.00 | 25.33 |
| ATOM | 2913 | CD1 | ILE | B | 576 | 34.503 | 56.083 | 16.761 | 1.00 | 25.78 |
| ATOM | 2914 | C | ILE | B | 576 | 36.506 | 51.521 | 17.717 | 1.00 | 22.18 |
| ATOM | 2915 | O | ILE | B | 576 | 37.344 | 51.139 | 16.900 | 1.00 | 21.36 |
| ATOM | 2916 | N | ARG | B | 577 | 36.619 | 51.318 | 19.028 | 1.00 | 21.36 |
| ATOM | 2917 | CA | ARG | B | 577 | 37.761 | 50.620 | 19.598 | 1.00 | 22.50 |
| ATOM | 2918 | CB | ARG | B | 577 | 37.749 | 50.702 | 21.131 | 1.00 | 20.87 |
| ATOM | 2919 | CG | ARG | B | 577 | 38.879 | 49.924 | 21.796 | 1.00 | 21.90 |
| ATOM | 2920 | CD | ARG | B | 577 | 40.239 | 50.280 | 21.195 | 1.00 | 21.96 |
| ATOM | 2921 | NE | ARG | B | 577 | 40.657 | 51.648 | 21.501 | 1.00 | 21.18 |
| ATOM | 2922 | CZ | ARG | B | 577 | 41.693 | 52.255 | 20.927 | 1.00 | 22.44 |
| ATOM | 2923 | NH1 | ARG | B | 577 | 42.421 | 51.622 | 20.011 | 1.00 | 22.46 |
| ATOM | 2924 | NH2 | ARG | B | 577 | 42.005 | 53.504 | 21.265 | 1.00 | 22.38 |
| ATOM | 2925 | C | ARG | B | 577 | 37.779 | 49.164 | 19.149 | 1.00 | 21.04 |
| ATOM | 2926 | O | ARG | B | 577 | 38.846 | 48.599 | 18.938 | 1.00 | 23.18 |
| ATOM | 2927 | N | MET | B | 578 | 36.600 | 48.560 | 19.000 | 1.00 | 21.91 |
| ATOM | 2928 | CA | MET | B | 578 | 36.507 | 47.172 | 18.548 | 1.00 | 21.13 |
| ATOM | 2929 | CB | MET | B | 578 | 35.054 | 46.705 | 18.554 | 1.00 | 20.87 |
| ATOM | 2930 | CG | MET | B | 578 | 34.503 | 46.404 | 19.925 | 1.00 | 21.65 |
| ATOM | 2931 | SD | MET | B | 578 | 32.749 | 46.041 | 19.841 | 1.00 | 22.25 |
| ATOM | 2932 | CE | MET | B | 578 | 32.205 | 46.673 | 21.420 | 1.00 | 22.99 |
| ATOM | 2933 | C | MET | B | 578 | 37.079 | 47.018 | 17.135 | 1.00 | 21.37 |
| ATOM | 2934 | O | MET | B | 578 | 37.872 | 46.114 | 16.876 | 1.00 | 21.93 |
| ATOM | 2935 | N | PHE | B | 579 | 36.660 | 47.886 | 16.222 | 1.00 | 21.44 |
| ATOM | 2936 | CA | PHE | B | 579 | 37.160 | 47.840 | 14.850 | 1.00 | 20.75 |
| ATOM | 2937 | CB | PHE | B | 579 | 36.496 | 48.908 | 13.979 | 1.00 | 23.89 |
| ATOM | 2938 | CG | PHE | B | 579 | 35.160 | 48.512 | 13.440 | 1.00 | 22.77 |
| ATOM | 2939 | CD1 | PHE | B | 579 | 34.009 | 48.672 | 14.203 | 1.00 | 22.94 |
| ATOM | 2940 | CD2 | PHE | B | 579 | 35.051 | 47.979 | 12.161 | 1.00 | 22.86 |
| ATOM | 2941 | CE1 | PHE | B | 579 | 32.765 | 48.302 | 13.695 | 1.00 | 23.49 |
| ATOM | 2942 | CE2 | PHE | B | 579 | 33.813 | 47.607 | 11.645 | 1.00 | 23.55 |
| ATOM | 2943 | CZ | PHE | B | 579 | 32.668 | 47.769 | 12.415 | 1.00 | 23.73 |
| ATOM | 2944 | C | PHE | B | 579 | 38.660 | 48.101 | 14.832 | 1.00 | 21.82 |
| ATOM | 2945 | O | PHE | B | 579 | 39.411 | 47.442 | 14.113 | 1.00 | 20.97 |
| ATOM | 2946 | N | THR | B | 580 | 39.085 | 49.074 | 15.628 | 1.00 | 23.77 |
| ATOM | 2947 | CA | THR | B | 580 | 40.488 | 49.450 | 15.684 | 1.00 | 23.88 |
| ATOM | 2948 | CB | THR | B | 580 | 40.684 | 50.693 | 16.572 | 1.00 | 24.40 |
| ATOM | 2949 | OG1 | THR | B | 580 | 39.895 | 51.770 | 16.051 | 1.00 | 25.13 |
| ATOM | 2950 | CG2 | THR | B | 580 | 42.153 | 51.109 | 16.596 | 1.00 | 24.68 |
| ATOM | 2951 | C | THR | B | 580 | 41.402 | 48.338 | 16.174 | 1.00 | 24.19 |
| ATOM | 2952 | O | THR | B | 580 | 42.389 | 48.011 | 15.512 | 1.00 | 24.60 |
| ATOM | 2953 | N | ASP | B | 581 | 41.072 | 47.743 | 17.314 | 1.00 | 26.39 |
| ATOM | 2954 | CA | ASP | B | 581 | 41.903 | 46.687 | 17.872 | 1.00 | 27.24 |
| ATOM | 2955 | CB | ASP | B | 581 | 41.635 | 46.554 | 19.369 | 1.00 | 26.21 |
| ATOM | 2956 | CG | ASP | B | 581 | 42.295 | 47.666 | 20.171 | 1.00 | 26.06 |
| ATOM | 2957 | OD1 | ASP | B | 581 | 42.688 | 48.690 | 19.565 | 1.00 | 25.91 |
| ATOM | 2958 | OD2 | ASP | B | 581 | 42.418 | 47.520 | 21.402 | 1.00 | 26.26 |
| ATOM | 2959 | C | ASP | B | 581 | 41.821 | 45.333 | 17.182 | 1.00 | 27.49 |
| ATOM | 2960 | O | ASP | B | 581 | 42.535 | 44.403 | 17.552 | 1.00 | 27.57 |
| ATOM | 2961 | N | LEU | B | 582 | 40.947 | 45.217 | 16.189 | 1.00 | 23.58 |
| ATOM | 2962 | CA | LEU | B | 582 | 40.859 | 43.984 | 15.415 | 1.00 | 24.50 |
| ATOM | 2963 | CB | LEU | B | 582 | 39.395 | 43.600 | 15.139 | 1.00 | 22.05 |
| ATOM | 2964 | CG | LEU | B | 582 | 38.660 | 42.981 | 16.337 | 1.00 | 21.75 |
| ATOM | 2965 | CD1 | LEU | B | 582 | 37.178 | 42.787 | 16.035 | 1.00 | 22.80 |
| ATOM | 2966 | CD2 | LEU | B | 582 | 39.309 | 41.648 | 16.680 | 1.00 | 21.90 |
| ATOM | 2967 | C | LEU | B | 582 | 41.605 | 44.305 | 14.117 | 1.00 | 24.79 |
| ATOM | 2968 | O | LEU | B | 582 | 41.591 | 43.531 | 13.153 | 1.00 | 25.28 |
| ATOM | 2969 | N | ASN | B | 583 | 42.255 | 45.469 | 14.117 | 1.00 | 27.83 |
| ATOM | 2970 | CA | ASN | B | 583 | 43.037 | 45.956 | 12.984 | 1.00 | 30.24 |
| ATOM | 2971 | CB | ASN | B | 583 | 44.235 | 45.037 | 12.740 | 1.00 | 52.59 |
| ATOM | 2972 | CG | ASN | B | 583 | 45.196 | 45.017 | 13.906 | 1.00 | 53.50 |
| ATOM | 2973 | OD1 | ASN | B | 583 | 44.827 | 44.655 | 15.023 | 1.00 | 53.73 |
| ATOM | 2974 | ND2 | ASN | B | 583 | 46.440 | 45.410 | 13.655 | 1.00 | 53.71 |
| ATOM | 2975 | C | ASN | B | 583 | 42.223 | 46.074 | 11.704 | 1.00 | 29.60 |
| ATOM | 2976 | O | ASN | B | 583 | 42.733 | 45.818 | 10.614 | 1.00 | 30.55 |
| ATOM | 2977 | N | LEU | B | 584 | 40.964 | 46.478 | 11.840 | 1.00 | 27.68 |
| ATOM | 2978 | CA | LEU | B | 584 | 40.069 | 46.620 | 10.696 | 1.00 | 27.10 |
| ATOM | 2979 | CB | LEU | B | 584 | 38.632 | 46.323 | 11.131 | 1.00 | 25.59 |
| ATOM | 2980 | CG | LEU | B | 584 | 38.111 | 44.884 | 11.080 | 1.00 | 26.48 |
| ATOM | 2981 | CD1 | LEU | B | 584 | 39.198 | 43.891 | 11.360 | 1.00 | 26.71 |
| ATOM | 2982 | CD2 | LEU | B | 584 | 36.954 | 44.746 | 12.065 | 1.00 | 24.71 |
| ATOM | 2983 | C | LEU | B | 584 | 40.115 | 47.992 | 10.047 | 1.00 | 27.65 |
| ATOM | 2984 | O | LEU | B | 584 | 39.908 | 48.120 | 8.843 | 1.00 | 26.67 |
| ATOM | 2985 | N | VAL | B | 585 | 40.380 | 49.021 | 10.845 | 1.00 | 28.02 |
| ATOM | 2986 | CA | VAL | B | 585 | 40.415 | 50.380 | 10.326 | 1.00 | 28.97 |
| ATOM | 2987 | CB | VAL | B | 585 | 40.586 | 51.400 | 11.467 | 1.00 | 33.37 |
| ATOM | 2988 | CG1 | VAL | B | 585 | 40.639 | 52.812 | 10.902 | 1.00 | 33.53 |
| ATOM | 2989 | CG2 | VAL | B | 585 | 39.436 | 51.267 | 12.448 | 1.00 | 33.91 |
| ATOM | 2990 | C | VAL | B | 585 | 41.502 | 50.620 | 9.283 | 1.00 | 29.73 |
| ATOM | 2991 | O | VAL | B | 585 | 41.210 | 51.056 | 8.172 | 1.00 | 29.62 |
| ATOM | 2992 | N | GLN | B | 586 | 42.749 | 50.330 | 9.638 | 1.00 | 34.73 |
| ATOM | 2993 | CA | GLN | B | 586 | 43.860 | 50.536 | 8.718 | 1.00 | 36.38 |
| ATOM | 2994 | CB | GLN | B | 586 | 45.171 | 50.626 | 9.501 | 1.00 | 56.06 |
| ATOM | 2995 | CG | GLN | B | 586 | 45.261 | 51.887 | 10.349 | 1.00 | 56.60 |
| ATOM | 2996 | CD | GLN | B | 586 | 46.523 | 51.955 | 11.184 | 1.00 | 57.01 |
| ATOM | 2997 | OE1 | GLN | B | 586 | 46.729 | 51.142 | 12.085 | 1.00 | 56.81 |
| ATOM | 2998 | NE2 | GLN | B | 586 | 47.378 | 52.930 | 10.887 | 1.00 | 56.83 |
| ATOM | 2999 | C | GLN | B | 586 | 43.936 | 49.439 | 7.666 | 1.00 | 35.19 |
| ATOM | 3000 | O | GLN | B | 586 | 44.176 | 49.716 | 6.492 | 1.00 | 36.38 |
| ATOM | 3001 | N | ASN | B | 587 | 43.716 | 48.199 | 8.088 | 1.00 | 28.60 |
| ATOM | 3002 | CA | ASN | B | 587 | 43.749 | 47.058 | 7.180 | 1.00 | 27.39 |
| ATOM | 3003 | CB | ASN | B | 587 | 43.208 | 45.816 | 7.893 | 1.00 | 34.50 |
| ATOM | 3004 | CG | ASN | B | 587 | 43.520 | 44.530 | 7.155 | 1.00 | 34.35 |
| ATOM | 3005 | OD1 | ASN | B | 587 | 43.721 | 44.528 | 5.943 | 1.00 | 34.55 |
| ATOM | 3006 | ND2 | ASN | B | 587 | 43.549 | 43.423 | 7.888 | 1.00 | 34.96 |
| ATOM | 3007 | C | ASN | B | 587 | 42.895 | 47.331 | 5.941 | 1.00 | 27.36 |
| ATOM | 3008 | O | ASN | B | 587 | 43.336 | 47.133 | 4.811 | 1.00 | 26.90 |
| ATOM | 3009 | N | PHE | B | 588 | 41.666 | 47.796 | 6.154 | 1.00 | 26.94 |
| ATOM | 3010 | CA | PHE | B | 588 | 40.765 | 48.048 | 5.040 | 1.00 | 26.43 |
| ATOM | 3011 | CB | PHE | B | 588 | 39.423 | 47.350 | 5.317 | 1.00 | 20.94 |
| ATOM | 3012 | CG | PHE | B | 588 | 39.558 | 45.856 | 5.503 | 1.00 | 20.97 |
| ATOM | 3013 | CD1 | PHE | B | 588 | 39.472 | 45.277 | 6.765 | 1.00 | 20.43 |
| ATOM | 3014 | CD2 | PHE | B | 588 | 39.845 | 45.038 | 4.417 | 1.00 | 20.60 |
| ATOM | 3015 | CE1 | PHE | B | 588 | 39.678 | 43.899 | 6.940 | 1.00 | 20.54 |
| ATOM | 3016 | CE2 | PHE | B | 588 | 40.052 | 43.662 | 4.576 | 1.00 | 19.56 |
| ATOM | 3017 | CZ | PHE | B | 588 | 39.969 | 43.091 | 5.842 | 1.00 | 21.12 |
| ATOM | 3018 | C | PHE | B | 588 | 40.573 | 49.521 | 4.670 | 1.00 | 27.33 |
| ATOM | 3019 | O | PHE | B | 588 | 39.625 | 49.878 | 3.973 | 1.00 | 27.04 |
| ATOM | 3020 | N | GLN | B | 589 | 41.502 | 50.365 | 5.111 | 1.00 | 28.79 |
| ATOM | 3021 | CA | GLN | B | 589 | 41.465 | 51.800 | 4.818 | 1.00 | 29.49 |
| ATOM | 3022 | CB | GLN | B | 589 | 41.843 | 52.064 | 3.350 | 1.00 | 45.46 |
| ATOM | 3023 | CG | GLN | B | 589 | 43.334 | 51.986 | 3.014 | 1.00 | 45.82 |
| ATOM | 3024 | CD | GLN | B | 589 | 43.797 | 50.588 | 2.639 | 1.00 | 46.29 |
| ATOM | 3025 | OE1 | GLN | B | 589 | 42.993 | 49.736 | 2.257 | 1.00 | 46.04 |
| ATOM | 3026 | NE2 | GLN | B | 589 | 45.104 | 50.353 | 2.724 | 1.00 | 45.90 |
| ATOM | 3027 | C | GLN | B | 589 | 40.127 | 52.481 | 5.106 | 1.00 | 29.77 |
| ATOM | 3028 | O | GLN | B | 589 | 39.509 | 53.063 | 4.212 | 1.00 | 29.22 |
| ATOM | 3029 | N | MET | B | 590 | 39.686 | 52.421 | 6.357 | 1.00 | 30.86 |
| ATOM | 3030 | CA | MET | B | 590 | 38.432 | 53.050 | 6.743 | 1.00 | 30.28 |
| ATOM | 3031 | CB | MET | B | 590 | 37.759 | 52.259 | 7.865 | 1.00 | 26.11 |
| ATOM | 3032 | CG | MET | B | 590 | 37.254 | 50.888 | 7.485 | 1.00 | 26.11 |
| ATOM | 3033 | SD | MET | B | 590 | 36.366 | 50.192 | 8.891 | 1.00 | 24.81 |
| ATOM | 3034 | CE | MET | B | 590 | 35.005 | 51.232 | 8.953 | 1.00 | 27.02 |
| ATOM | 3035 | C | MET | B | 590 | 38.680 | 54.467 | 7.241 | 1.00 | 30.44 |
| ATOM | 3036 | O | MET | B | 590 | 39.453 | 54.666 | 8.175 | 1.00 | 30.43 |
| ATOM | 3037 | N | LYS | B | 591 | 38.039 | 55.449 | 6.616 | 1.00 | 28.66 |
| ATOM | 3038 | CA | LYS | B | 591 | 38.181 | 56.833 | 7.059 | 1.00 | 29.65 |
| ATOM | 3039 | CB | LYS | B | 591 | 37.556 | 57.798 | 6.045 | 1.00 | 48.54 |
| ATOM | 3040 | CG | LYS | B | 591 | 38.530 | 58.376 | 5.022 | 1.00 | 49.40 |
| ATOM | 3041 | CD | LYS | B | 591 | 39.125 | 57.314 | 4.114 | 1.00 | 49.46 |
| ATOM | 3042 | CE | LYS | B | 591 | 40.123 | 57.925 | 3.141 | 1.00 | 49.63 |
| ATOM | 3043 | NZ | LYS | B | 591 | 39.519 | 59.026 | 2.336 | 1.00 | 49.46 |
| ATOM | 3044 | C | LYS | B | 591 | 37.453 | 56.951 | 8.399 | 1.00 | 28.39 |
| ATOM | 3045 | O | LYS | B | 591 | 36.361 | 56.409 | 8.566 | 1.00 | 28.80 |
| ATOM | 3046 | N | HIS | B | 592 | 38.060 | 57.646 | 9.354 | 1.00 | 26.99 |
| ATOM | 3047 | CA | HIS | B | 592 | 37.447 | 57.803 | 10.669 | 1.00 | 26.60 |
| ATOM | 3048 | CB | HIS | B | 592 | 38.305 | 58.713 | 11.554 | 1.00 | 26.53 |
| ATOM | 3049 | CG | HIS | B | 592 | 37.869 | 58.742 | 12.985 | 1.00 | 26.27 |
| ATOM | 3050 | CD2 | HIS | B | 592 | 37.395 | 59.750 | 13.755 | 1.00 | 26.34 |
| ATOM | 3051 | ND1 | HIS | B | 592 | 37.888 | 57.622 | 13.788 | 1.00 | 26.28 |
| ATOM | 3052 | CE1 | HIS | B | 592 | 37.446 | 57.939 | 14.992 | 1.00 | 26.55 |
| ATOM | 3053 | NE2 | HIS | B | 592 | 37.140 | 59.224 | 14.998 | 1.00 | 26.20 |
| ATOM | 3054 | C | HIS | B | 592 | 36.027 | 58.366 | 10.594 | 1.00 | 26.93 |
| ATOM | 3055 | O | HIS | B | 592 | 35.114 | 57.856 | 11.244 | 1.00 | 26.72 |
| ATOM | 3056 | N | GLU | B | 593 | 35.844 | 59.423 | 9.810 | 1.00 | 25.99 |
| ATOM | 3057 | CA | GLU | B | 593 | 34.532 | 60.049 | 9.675 | 1.00 | 26.68 |
| ATOM | 3058 | CB | GLU | B | 593 | 34.615 | 61.240 | 8.717 | 1.00 | 47.65 |
| ATOM | 3059 | CG | GLU | B | 593 | 35.479 | 62.386 | 9.232 | 1.00 | 48.65 |
| ATOM | 3060 | CD | GLU | B | 593 | 36.880 | 61.942 | 9.628 | 1.00 | 48.66 |
| ATOM | 3061 | OE1 | GLU | B | 593 | 37.595 | 61.373 | 8.773 | 1.00 | 48.66 |
| ATOM | 3062 | OE2 | GLU | B | 593 | 37.266 | 62.160 | 10.798 | 1.00 | 48.65 |
| ATOM | 3063 | C | GLU | B | 593 | 33.479 | 59.056 | 9.188 | 1.00 | 25.92 |
| ATOM | 3064 | O | GLU | B | 593 | 32.368 | 59.011 | 9.717 | 1.00 | 25.44 |
| ATOM | 3065 | N | VAL | B | 594 | 33.837 | 58.257 | 8.188 | 1.00 | 23.11 |
| ATOM | 3066 | CA | VAL | B | 594 | 32.914 | 57.265 | 7.640 | 1.00 | 23.96 |
| ATOM | 3067 | CB | VAL | B | 594 | 33.511 | 56.574 | 6.390 | 1.00 | 21.56 |
| ATOM | 3068 | CG1 | VAL | B | 594 | 32.576 | 55.471 | 5.891 | 1.00 | 21.95 |
| ATOM | 3069 | CG2 | VAL | B | 594 | 33.747 | 57.604 | 5.297 | 1.00 | 21.81 |
| ATOM | 3070 | C | VAL | B | 594 | 32.556 | 56.196 | 8.667 | 1.00 | 23.30 |
| ATOM | 3071 | O | VAL | B | 594 | 31.385 | 55.849 | 8.824 | 1.00 | 23.33 |
| ATOM | 3072 | N | LEU | B | 595 | 33.558 | 55.663 | 9.360 | 1.00 | 24.80 |
| ATOM | 3073 | CA | LEU | B | 595 | 33.300 | 54.639 | 10.364 | 1.00 | 24.45 |
| ATOM | 3074 | CB | LEU | B | 595 | 34.616 | 54.127 | 10.959 | 1.00 | 24.30 |
| ATOM | 3075 | CG | LEU | B | 595 | 34.502 | 53.137 | 12.123 | 1.00 | 25.09 |
| ATOM | 3076 | CD1 | LEU | B | 595 | 33.643 | 51.938 | 11.723 | 1.00 | 23.72 |
| ATOM | 3077 | CD2 | LEU | B | 595 | 35.892 | 52.686 | 12.545 | 1.00 | 23.89 |
| ATOM | 3078 | C | LEU | B | 595 | 32.391 | 55.190 | 11.464 | 1.00 | 24.32 |
| ATOM | 3079 | O | LEU | B | 595 | 31.482 | 54.501 | 11.935 | 1.00 | 24.28 |
| ATOM | 3080 | N | CYS | B | 596 | 32.625 | 56.437 | 11.866 | 1.00 | 23.70 |
| ATOM | 3081 | CA | CYS | B | 596 | 31.794 | 57.057 | 12.895 | 1.00 | 22.77 |
| ATOM | 3082 | CB | CYS | B | 596 | 32.354 | 58.426 | 13.302 | 1.00 | 27.45 |
| ATOM | 3083 | SG | CYS | B | 596 | 33.875 | 58.384 | 14.277 | 1.00 | 27.23 |
| ATOM | 3084 | C | CYS | B | 596 | 30.363 | 57.233 | 12.386 | 1.00 | 22.78 |
| ATOM | 3085 | O | CYS | B | 596 | 29.404 | 56.963 | 13.108 | 1.00 | 22.99 |
| ATOM | 3086 | N | ARG | B | 597 | 30.221 | 57.681 | 11.140 | 1.00 | 22.21 |
| ATOM | 3087 | CA | ARG | B | 597 | 28.899 | 57.893 | 10.562 | 1.00 | 21.97 |
| ATOM | 3088 | CB | ARG | B | 597 | 29.018 | 58.543 | 9.178 | 1.00 | 33.99 |
| ATOM | 3089 | CG | ARG | B | 597 | 27.678 | 58.924 | 8.570 | 1.00 | 35.29 |
| ATOM | 3090 | CD | ARG | B | 597 | 27.807 | 59.645 | 7.228 | 1.00 | 35.28 |
| ATOM | 3091 | NE | ARG | B | 597 | 28.241 | 61.035 | 7.362 | 1.00 | 35.24 |
| ATOM | 3092 | CZ | ARG | B | 597 | 29.494 | 61.455 | 7.216 | 1.00 | 35.37 |
| ATOM | 3093 | NH1 | ARG | B | 597 | 30.463 | 60.596 | 6.926 | 1.00 | 35.79 |
| ATOM | 3094 | NH2 | ARG | B | 597 | 29.778 | 62.745 | 7.354 | 1.00 | 35.68 |
| ATOM | 3095 | C | ARG | B | 597 | 28.149 | 56.563 | 10.459 | 1.00 | 21.87 |
| ATOM | 3096 | O | ARG | B | 597 | 26.954 | 56.496 | 10.729 | 1.00 | 21.94 |
| ATOM | 3097 | N | TRP | B | 598 | 28.862 | 55.511 | 10.069 | 1.00 | 23.25 |
| ATOM | 3098 | CA | TRP | B | 598 | 28.264 | 54.185 | 9.944 | 1.00 | 23.59 |
| ATOM | 3099 | CB | TRP | B | 598 | 29.287 | 53.194 | 9.374 | 1.00 | 21.37 |
| ATOM | 3100 | CG | TRP | B | 598 | 28.744 | 51.811 | 9.234 | 1.00 | 19.95 |
| ATOM | 3101 | CD2 | TRP | B | 598 | 28.905 | 50.725 | 10.157 | 1.00 | 20.76 |
| ATOM | 3102 | CE2 | TRP | B | 598 | 28.159 | 49.636 | 9.653 | 1.00 | 21.16 |
| ATOM | 3103 | CE3 | TRP | B | 598 | 29.602 | 50.568 | 11.363 | 1.00 | 20.58 |
| ATOM | 3104 | CD1 | TRP | B | 598 | 27.937 | 51.349 | 8.237 | 1.00 | 20.72 |
| ATOM | 3105 | NE1 | TRP | B | 598 | 27.581 | 50.044 | 8.481 | 1.00 | 20.01 |
| ATOM | 3106 | CZ2 | TRP | B | 598 | 28.091 | 48.404 | 10.313 | 1.00 | 19.36 |
| ATOM | 3107 | CZ3 | TRP | B | 598 | 29.534 | 49.346 | 12.019 | 1.00 | 21.53 |
| ATOM | 3108 | CH2 | TRP | B | 598 | 28.780 | 48.277 | 11.491 | 1.00 | 21.51 |
| ATOM | 3109 | C | TRP | B | 598 | 27.774 | 53.687 | 11.301 | 1.00 | 23.33 |
| ATOM | 3110 | O | TRP | B | 598 | 26.660 | 53.170 | 11.429 | 1.00 | 22.55 |
| ATOM | 3111 | N | ILE | B | 599 | 28.614 | 53.836 | 12.318 | 1.00 | 19.88 |
| ATOM | 3112 | CA | ILE | B | 599 | 28.250 | 53.401 | 13.652 | 1.00 | 19.76 |
| ATOM | 3113 | CB | ILE | B | 599 | 29.400 | 53.659 | 14.656 | 1.00 | 18.66 |
| ATOM | 3114 | CG2 | ILE | B | 599 | 28.917 | 53.410 | 16.078 | 1.00 | 20.09 |
| ATOM | 3115 | CG1 | ILE | B | 599 | 30.586 | 52.742 | 14.328 | 1.00 | 19.91 |
| ATOM | 3116 | CD1 | ILE | B | 599 | 31.835 | 53.042 | 15.140 | 1.00 | 18.69 |
| ATOM | 3117 | C | ILE | B | 599 | 26.989 | 54.124 | 14.105 | 1.00 | 20.60 |
| ATOM | 3118 | O | ILE | B | 599 | 26.072 | 53.501 | 14.636 | 1.00 | 20.28 |
| ATOM | 3119 | N | LEU | B | 600 | 26.933 | 55.435 | 13.883 | 1.00 | 24.36 |
| ATOM | 3120 | CA | LEU | B | 600 | 25.761 | 56.200 | 14.292 | 1.00 | 24.27 |
| ATOM | 3121 | CB | LEU | B | 600 | 26.023 | 57.700 | 14.135 | 1.00 | 30.88 |
| ATOM | 3122 | CG | LEU | B | 600 | 27.104 | 58.235 | 15.080 | 1.00 | 31.45 |
| ATOM | 3123 | CD1 | LEU | B | 600 | 27.378 | 59.680 | 14.764 | 1.00 | 31.56 |
| ATOM | 3124 | CD2 | LEU | B | 600 | 26.663 | 58.079 | 16.529 | 1.00 | 31.65 |
| ATOM | 3125 | C | LEU | B | 600 | 24.518 | 55.788 | 13.512 | 1.00 | 25.39 |
| ATOM | 3126 | O | LEU | B | 600 | 23.432 | 55.697 | 14.086 | 1.00 | 24.82 |
| ATOM | 3127 | N | SER | B | 601 | 24.681 | 55.522 | 12.216 | 1.00 | 24.46 |
| ATOM | 3128 | CA | SER | B | 601 | 23.563 | 55.098 | 11.373 | 1.00 | 23.76 |
| ATOM | 3129 | CB | SER | B | 601 | 24.018 | 54.931 | 9.920 | 1.00 | 24.34 |
| ATOM | 3130 | OG | SER | B | 601 | 24.232 | 56.181 | 9.287 | 1.00 | 24.61 |
| ATOM | 3131 | C | SER | B | 601 | 23.004 | 53.771 | 11.884 | 1.00 | 23.31 |
| ATOM | 3132 | O | SER | B | 601 | 21.793 | 53.574 | 11.947 | 1.00 | 24.08 |
| ATOM | 3133 | N | VAL | B | 602 | 23.895 | 52.855 | 12.248 | 1.00 | 20.00 |
| ATOM | 3134 | CA | VAL | B | 602 | 23.465 | 51.560 | 12.765 | 1.00 | 19.52 |
| ATOM | 3135 | CB | VAL | B | 602 | 24.681 | 50.622 | 12.998 | 1.00 | 20.56 |
| ATOM | 3136 | CG1 | VAL | B | 602 | 24.271 | 49.399 | 13.806 | 1.00 | 20.50 |
| ATOM | 3137 | CG2 | VAL | B | 602 | 25.258 | 50.193 | 11.659 | 1.00 | 20.79 |
| ATOM | 3138 | C | VAL | B | 602 | 22.685 | 51.742 | 14.073 | 1.00 | 18.74 |
| ATOM | 3139 | O | VAL | B | 602 | 21.598 | 51.188 | 14.243 | 1.00 | 19.51 |
| ATOM | 3140 | N | LYS | B | 603 | 23.230 | 52.533 | 14.989 | 1.00 | 21.59 |
| ATOM | 3141 | CA | LYS | B | 603 | 22.568 | 52.767 | 16.268 | 1.00 | 22.24 |
| ATOM | 3142 | CB | LYS | B | 603 | 23.433 | 53.674 | 17.156 | 1.00 | 32.07 |
| ATOM | 3143 | CG | LYS | B | 603 | 22.812 | 53.968 | 18.523 | 1.00 | 32.67 |
| ATOM | 3144 | CD | LYS | B | 603 | 23.775 | 54.700 | 19.457 | 1.00 | 32.26 |
| ATOM | 3145 | CE | LYS | B | 603 | 24.152 | 56.067 | 18.910 | 1.00 | 33.03 |
| ATOM | 3146 | NZ | LYS | B | 603 | 24.991 | 56.838 | 19.866 | 1.00 | 33.90 |
| ATOM | 3147 | C | LYS | B | 603 | 21.193 | 53.404 | 16.072 | 1.00 | 21.31 |
| ATOM | 3148 | O | LYS | B | 603 | 20.229 | 53.057 | 16.758 | 1.00 | 21.06 |
| ATOM | 3149 | N | LYS | B | 604 | 21.103 | 54.318 | 15.116 | 1.00 | 21.77 |
| ATOM | 3150 | CA | LYS | B | 604 | 19.846 | 55.003 | 14.860 | 1.00 | 22.10 |
| ATOM | 3151 | CB | LYS | B | 604 | 20.071 | 56.179 | 13.904 | 1.00 | 35.05 |
| ATOM | 3152 | CG | LYS | B | 604 | 18.815 | 56.987 | 13.614 | 1.00 | 35.34 |
| ATOM | 3153 | CD | LYS | B | 604 | 19.111 | 58.153 | 12.691 | 1.00 | 35.68 |
| ATOM | 3154 | CE | LYS | B | 604 | 17.841 | 58.897 | 12.313 | 1.00 | 35.70 |
| ATOM | 3155 | NZ | LYS | B | 604 | 18.118 | 60.009 | 11.357 | 1.00 | 35.94 |
| ATOM | 3156 | C | LYS | B | 604 | 18.781 | 54.088 | 14.290 | 1.00 | 23.45 |
| ATOM | 3157 | O | LYS | B | 604 | 17.588 | 54.266 | 14.567 | 1.00 | 23.47 |
| ATOM | 3158 | N | ASN | B | 605 | 19.200 | 53.104 | 13.497 | 1.00 | 24.27 |
| ATOM | 3159 | CA | ASN | B | 605 | 18.238 | 52.198 | 12.887 | 1.00 | 24.84 |
| ATOM | 3160 | CB | ASN | B | 605 | 18.790 | 51.629 | 11.580 | 1.00 | 33.82 |
| ATOM | 3161 | CG | ASN | B | 605 | 18.757 | 52.650 | 10.461 | 1.00 | 34.72 |
| ATOM | 3162 | OD1 | ASN | B | 605 | 19.593 | 53.552 | 10.401 | 1.00 | 35.05 |
| ATOM | 3163 | ND2 | ASN | B | 605 | 17.771 | 52.529 | 9.579 | 1.00 | 34.67 |
| ATOM | 3164 | C | ASN | B | 605 | 17.713 | 51.091 | 13.784 | 1.00 | 25.28 |
| ATOM | 3165 | O | ASN | B | 605 | 17.138 | 50.109 | 13.309 | 1.00 | 26.57 |
| ATOM | 3166 | N | TYR | B | 606 | 17.935 | 51.237 | 15.083 | 1.00 | 22.80 |
| ATOM | 3167 | CA | TYR | B | 606 | 17.381 | 50.301 | 16.047 | 1.00 | 21.47 |
| ATOM | 3168 | CB | TYR | B | 606 | 18.406 | 49.906 | 17.116 | 1.00 | 19.39 |
| ATOM | 3169 | CG | TYR | B | 606 | 19.259 | 48.734 | 16.684 | 1.00 | 18.74 |
| ATOM | 3170 | CD1 | TYR | B | 606 | 20.482 | 48.930 | 16.047 | 1.00 | 19.52 |
| ATOM | 3171 | CE1 | TYR | B | 606 | 21.221 | 47.852 | 15.566 | 1.00 | 19.69 |
| ATOM | 3172 | CD2 | TYR | B | 606 | 18.795 | 47.427 | 16.836 | 1.00 | 18.61 |
| ATOM | 3173 | CE2 | TYR | B | 606 | 19.530 | 46.337 | 16.355 | 1.00 | 19.30 |
| ATOM | 3174 | CZ | TYR | B | 606 | 20.739 | 46.563 | 15.719 | 1.00 | 19.25 |
| ATOM | 3175 | OH | TYR | B | 606 | 21.459 | 45.499 | 15.209 | 1.00 | 18.12 |
| ATOM | 3176 | C | TYR | B | 606 | 16.234 | 51.093 | 16.662 | 1.00 | 22.67 |
| ATOM | 3177 | O | TYR | B | 606 | 16.323 | 52.319 | 16.782 | 1.00 | 24.05 |
| ATOM | 3178 | N | ARG | B | 607 | 15.146 | 50.419 | 17.009 | 1.00 | 23.34 |
| ATOM | 3179 | CA | ARG | B | 607 | 13.997 | 51.106 | 17.602 | 1.00 | 23.02 |
| ATOM | 3180 | CB | ARG | B | 607 | 12.718 | 50.315 | 17.322 | 1.00 | 22.58 |
| ATOM | 3181 | CG | ARG | B | 607 | 12.516 | 50.043 | 15.838 | 1.00 | 22.23 |
| ATOM | 3182 | CD | ARG | B | 607 | 11.149 | 49.467 | 15.533 | 1.00 | 23.26 |
| ATOM | 3183 | NE | ARG | B | 607 | 11.044 | 49.076 | 14.131 | 1.00 | 21.78 |
| ATOM | 3184 | CZ | ARG | B | 607 | 9.916 | 48.698 | 13.537 | 1.00 | 23.35 |
| ATOM | 3185 | NH1 | ARG | B | 607 | 8.780 | 48.661 | 14.220 | 1.00 | 22.77 |
| ATOM | 3186 | NH2 | ARG | B | 607 | 9.931 | 48.344 | 12.258 | 1.00 | 23.68 |
| ATOM | 3187 | C | ARG | B | 607 | 14.247 | 51.243 | 19.100 | 1.00 | 23.25 |
| ATOM | 3188 | O | ARG | B | 607 | 14.123 | 50.282 | 19.855 | 1.00 | 22.89 |
| ATOM | 3189 | N | LYS | B | 608 | 14.602 | 52.450 | 19.526 | 1.00 | 26.06 |
| ATOM | 3190 | CA | LYS | B | 608 | 14.921 | 52.697 | 20.928 | 1.00 | 27.43 |
| ATOM | 3191 | CB | LYS | B | 608 | 15.228 | 54.183 | 21.147 | 1.00 | 43.34 |
| ATOM | 3192 | CG | LYS | B | 608 | 14.041 | 55.104 | 20.946 | 1.00 | 43.63 |
| ATOM | 3193 | CD | LYS | B | 608 | 14.394 | 56.538 | 21.307 | 1.00 | 43.77 |
| ATOM | 3194 | CE | LYS | B | 608 | 13.192 | 57.458 | 21.141 | 1.00 | 44.09 |
| ATOM | 3195 | NZ | LYS | B | 608 | 13.518 | 58.878 | 21.457 | 1.00 | 43.98 |
| ATOM | 3196 | C | LYS | B | 608 | 13.878 | 52.248 | 21.940 | 1.00 | 26.36 |
| ATOM | 3197 | O | LYS | B | 608 | 14.221 | 51.870 | 23.062 | 1.00 | 27.18 |
| ATOM | 3198 | N | ASN | B | 609 | 12.606 | 52.277 | 21.559 | 1.00 | 24.76 |
| ATOM | 3199 | CA | ASN | B | 609 | 11.560 | 51.890 | 22.490 | 1.00 | 25.81 |
| ATOM | 3200 | CB | ASN | B | 609 | 10.305 | 52.718 | 22.222 | 1.00 | 34.37 |
| ATOM | 3201 | CG | ASN | B | 609 | 10.558 | 54.201 | 22.378 | 1.00 | 35.05 |
| ATOM | 3202 | OD1 | ASN | B | 609 | 10.969 | 54.657 | 23.444 | 1.00 | 35.79 |
| ATOM | 3203 | ND2 | ASN | B | 609 | 10.326 | 54.961 | 21.315 | 1.00 | 35.63 |
| ATOM | 3204 | C | ASN | B | 609 | 11.226 | 50.406 | 22.548 | 1.00 | 24.40 |
| ATOM | 3205 | O | ASN | B | 609 | 10.336 | 49.993 | 23.294 | 1.00 | 24.72 |
| ATOM | 3206 | N | VAL | B | 610 | 11.925 | 49.596 | 21.759 | 1.00 | 22.64 |
| ATOM | 3207 | CA | VAL | B | 610 | 11.699 | 48.160 | 21.814 | 1.00 | 21.87 |
| ATOM | 3208 | CB | VAL | B | 610 | 12.205 | 47.456 | 20.536 | 1.00 | 21.44 |
| ATOM | 3209 | CG1 | VAL | B | 610 | 12.237 | 45.953 | 20.746 | 1.00 | 21.17 |
| ATOM | 3210 | CG2 | VAL | B | 610 | 11.276 | 47.783 | 19.377 | 1.00 | 21.07 |
| ATOM | 3211 | C | VAL | B | 610 | 12.513 | 47.729 | 23.037 | 1.00 | 22.60 |
| ATOM | 3212 | O | VAL | B | 610 | 13.704 | 48.016 | 23.127 | 1.00 | 22.78 |
| ATOM | 3213 | N | ALA | B | 611 | 11.865 | 47.051 | 23.976 | 1.00 | 21.43 |
| ATOM | 3214 | CA | ALA | B | 611 | 12.500 | 46.651 | 25.232 | 1.00 | 21.58 |
| ATOM | 3215 | CB | ALA | B | 611 | 11.521 | 45.828 | 26.061 | 1.00 | 24.34 |
| ATOM | 3216 | C | ALA | B | 611 | 13.851 | 45.941 | 25.176 | 1.00 | 21.37 |
| ATOM | 3217 | O | ALA | B | 611 | 14.795 | 46.361 | 25.851 | 1.00 | 21.07 |
| ATOM | 3218 | N | TYR | B | 612 | 13.951 | 44.878 | 24.382 | 1.00 | 20.78 |
| ATOM | 3219 | CA | TYR | B | 612 | 15.202 | 44.119 | 24.301 | 1.00 | 19.11 |
| ATOM | 3220 | CB | TYR | B | 612 | 14.926 | 42.648 | 24.623 | 1.00 | 19.45 |
| ATOM | 3221 | CG | TYR | B | 612 | 16.144 | 41.756 | 24.493 | 1.00 | 19.72 |
| ATOM | 3222 | CD1 | TYR | B | 612 | 16.194 | 40.756 | 23.523 | 1.00 | 19.82 |
| ATOM | 3223 | CE1 | TYR | B | 612 | 17.304 | 39.933 | 23.401 | 1.00 | 19.95 |
| ATOM | 3224 | CD2 | TYR | B | 612 | 17.238 | 41.914 | 25.337 | 1.00 | 19.48 |
| ATOM | 3225 | CE2 | TYR | B | 612 | 18.359 | 41.094 | 25.222 | 1.00 | 20.02 |
| ATOM | 3226 | CZ | TYR | B | 612 | 18.382 | 40.109 | 24.259 | 1.00 | 20.21 |
| ATOM | 3227 | OH | TYR | B | 612 | 19.471 | 39.271 | 24.168 | 1.00 | 20.28 |
| ATOM | 3228 | C | TYR | B | 612 | 15.994 | 44.218 | 22.994 | 1.00 | 20.06 |
| ATOM | 3229 | O | TYR | B | 612 | 17.203 | 44.465 | 23.027 | 1.00 | 19.27 |
| ATOM | 3230 | N | HIS | B | 613 | 15.338 | 44.021 | 21.851 | 1.00 | 20.51 |
| ATOM | 3231 | CA | HIS | B | 613 | 16.046 | 44.096 | 20.569 | 1.00 | 19.40 |
| ATOM | 3232 | CB | HIS | B | 613 | 15.310 | 43.290 | 19.493 | 1.00 | 21.37 |
| ATOM | 3233 | CG | HIS | B | 613 | 15.351 | 41.811 | 19.712 | 1.00 | 21.97 |
| ATOM | 3234 | CD2 | HIS | B | 613 | 16.338 | 40.907 | 19.503 | 1.00 | 21.44 |
| ATOM | 3235 | ND1 | HIS | B | 613 | 14.283 | 41.101 | 20.216 | 1.00 | 21.50 |
| ATOM | 3236 | CE1 | HIS | B | 613 | 14.608 | 39.824 | 20.307 | 1.00 | 22.37 |
| ATOM | 3237 | NE2 | HIS | B | 613 | 15.851 | 39.679 | 19.881 | 1.00 | 22.50 |
| ATOM | 3238 | C | HIS | B | 613 | 16.229 | 45.530 | 20.093 | 1.00 | 19.18 |
| ATOM | 3239 | O | HIS | B | 613 | 15.583 | 45.971 | 19.149 | 1.00 | 19.28 |
| ATOM | 3240 | N | ASN | B | 614 | 17.137 | 46.241 | 20.749 | 1.00 | 19.04 |
| ATOM | 3241 | CA | ASN | B | 614 | 17.429 | 47.631 | 20.425 | 1.00 | 19.60 |
| ATOM | 3242 | CB | ASN | B | 614 | 16.895 | 48.526 | 21.541 | 1.00 | 24.32 |
| ATOM | 3243 | CG | ASN | B | 614 | 17.245 | 47.996 | 22.914 | 1.00 | 24.73 |
| ATOM | 3244 | OD1 | ASN | B | 614 | 18.405 | 47.678 | 23.188 | 1.00 | 26.31 |
| ATOM | 3245 | ND2 | ASN | B | 614 | 16.247 | 47.890 | 23.787 | 1.00 | 24.39 |
| ATOM | 3246 | C | ASN | B | 614 | 18.948 | 47.761 | 20.311 | 1.00 | 19.61 |
| ATOM | 3247 | O | ASN | B | 614 | 19.658 | 46.757 | 20.336 | 1.00 | 19.88 |
| ATOM | 3248 | N | TRP | B | 615 | 19.451 | 48.986 | 20.188 | 1.00 | 20.60 |
| ATOM | 3249 | CA | TRP | B | 615 | 20.893 | 49.189 | 20.052 | 1.00 | 20.16 |
| ATOM | 3250 | CB | TRP | B | 615 | 21.219 | 50.686 | 20.022 | 1.00 | 22.74 |
| ATOM | 3251 | CG | TRP | B | 615 | 22.672 | 50.982 | 20.262 | 1.00 | 23.84 |
| ATOM | 3252 | CD2 | TRP | B | 615 | 23.773 | 50.638 | 19.412 | 1.00 | 23.87 |
| ATOM | 3253 | CE2 | TRP | B | 615 | 24.949 | 51.101 | 20.046 | 1.00 | 24.13 |
| ATOM | 3254 | CE3 | TRP | B | 615 | 23.882 | 49.985 | 18.179 | 1.00 | 24.34 |
| ATOM | 3255 | CD1 | TRP | B | 615 | 23.209 | 51.620 | 21.346 | 1.00 | 23.95 |
| ATOM | 3256 | NE1 | TRP | B | 615 | 24.574 | 51.694 | 21.224 | 1.00 | 24.06 |
| ATOM | 3257 | CZ2 | TRP | B | 615 | 26.222 | 50.932 | 19.485 | 1.00 | 24.74 |
| ATOM | 3258 | CZ3 | TRP | B | 615 | 25.152 | 49.816 | 17.618 | 1.00 | 24.67 |
| ATOM | 3259 | CH2 | TRP | B | 615 | 26.304 | 50.290 | 18.276 | 1.00 | 23.93 |
| ATOM | 3260 | C | TRP | B | 615 | 21.776 | 48.514 | 21.098 | 1.00 | 19.91 |
| ATOM | 3261 | O | TRP | B | 615 | 22.834 | 47.960 | 20.766 | 1.00 | 18.94 |
| ATOM | 3262 | N | ARG | B | 616 | 21.373 | 48.572 | 22.361 | 1.00 | 18.07 |
| ATOM | 3263 | CA | ARG | B | 616 | 22.193 | 47.978 | 23.403 | 1.00 | 19.36 |
| ATOM | 3264 | CB | ARG | B | 616 | 21.610 | 48.286 | 24.785 | 1.00 | 22.36 |
| ATOM | 3265 | CG | ARG | B | 616 | 21.612 | 49.788 | 25.119 | 1.00 | 22.18 |
| ATOM | 3266 | CD | ARG | B | 616 | 23.029 | 50.377 | 25.052 | 1.00 | 22.30 |
| ATOM | 3267 | NE | ARG | B | 616 | 23.086 | 51.816 | 25.313 | 1.00 | 23.22 |
| ATOM | 3268 | CZ | ARG | B | 616 | 23.686 | 52.364 | 26.368 | 1.00 | 23.02 |
| ATOM | 3269 | NH1 | ARG | B | 616 | 24.280 | 51.600 | 27.272 | 1.00 | 22.92 |
| ATOM | 3270 | NH2 | ARG | B | 616 | 23.714 | 53.685 | 26.504 | 1.00 | 23.10 |
| ATOM | 3271 | C | ARG | B | 616 | 22.392 | 46.479 | 23.208 | 1.00 | 18.33 |
| ATOM | 3272 | O | ARG | B | 616 | 23.483 | 45.970 | 23.476 | 1.00 | 18.59 |
| ATOM | 3273 | N | HIS | B | 617 | 21.363 | 45.772 | 22.736 | 1.00 | 20.45 |
| ATOM | 3274 | CA | HIS | B | 617 | 21.495 | 44.333 | 22.512 | 1.00 | 17.68 |
| ATOM | 3275 | CB | HIS | B | 617 | 20.156 | 43.690 | 22.143 | 1.00 | 17.10 |
| ATOM | 3276 | CG | HIS | B | 617 | 20.300 | 42.309 | 21.571 | 1.00 | 16.72 |
| ATOM | 3277 | CD2 | HIS | B | 617 | 20.008 | 41.819 | 20.343 | 1.00 | 19.45 |
| ATOM | 3278 | ND1 | HIS | B | 617 | 20.842 | 41.261 | 22.283 | 1.00 | 17.70 |
| ATOM | 3279 | CE1 | HIS | B | 617 | 20.880 | 40.183 | 21.517 | 1.00 | 16.30 |
| ATOM | 3280 | NE2 | HIS | B | 617 | 20.380 | 40.497 | 20.336 | 1.00 | 14.56 |
| ATOM | 3281 | C | HIS | B | 617 | 22.507 | 44.058 | 21.402 | 1.00 | 18.17 |
| ATOM | 3282 | O | HIS | B | 617 | 23.343 | 43.151 | 21.528 | 1.00 | 17.84 |
| ATOM | 3283 | N | ALA | B | 618 | 22.420 | 44.836 | 20.326 | 1.00 | 18.42 |
| ATOM | 3284 | CA | ALA | B | 618 | 23.329 | 44.707 | 19.189 | 1.00 | 17.70 |
| ATOM | 3285 | CB | ALA | B | 618 | 22.918 | 45.659 | 18.067 | 1.00 | 21.95 |
| ATOM | 3286 | C | ALA | B | 618 | 24.749 | 45.026 | 19.652 | 1.00 | 17.93 |
| ATOM | 3287 | O | ALA | B | 618 | 25.701 | 44.320 | 19.324 | 1.00 | 18.41 |
| ATOM | 3288 | N | PHE | B | 619 | 24.882 | 46.100 | 20.421 | 1.00 | 17.19 |
| ATOM | 3289 | CA | PHE | B | 619 | 26.184 | 46.502 | 20.936 | 1.00 | 17.81 |
| ATOM | 3290 | CB | PHE | B | 619 | 26.040 | 47.811 | 21.726 | 1.00 | 18.98 |
| ATOM | 3291 | CG | PHE | B | 619 | 27.290 | 48.224 | 22.454 | 1.00 | 18.41 |
| ATOM | 3292 | CD1 | PHE | B | 619 | 28.423 | 48.626 | 21.755 | 1.00 | 18.73 |
| ATOM | 3293 | CD2 | PHE | B | 619 | 27.340 | 48.172 | 23.842 | 1.00 | 19.11 |
| ATOM | 3294 | CE1 | PHE | B | 619 | 29.588 | 48.966 | 22.427 | 1.00 | 19.47 |
| ATOM | 3295 | CE2 | PHE | B | 619 | 28.498 | 48.509 | 24.527 | 1.00 | 19.39 |
| ATOM | 3296 | CZ | PHE | B | 619 | 29.627 | 48.907 | 23.816 | 1.00 | 19.32 |
| ATOM | 3297 | C | PHE | B | 619 | 26.791 | 45.407 | 21.818 | 1.00 | 18.52 |
| ATOM | 3298 | O | PHE | B | 619 | 27.979 | 45.091 | 21.695 | 1.00 | 18.60 |
| ATOM | 3299 | N | ASN | B | 620 | 25.983 | 44.819 | 22.699 | 1.00 | 18.74 |
| ATOM | 3300 | CA | ASN | B | 620 | 26.484 | 43.774 | 23.585 | 1.00 | 19.38 |
| ATOM | 3301 | CB | ASN | B | 620 | 25.457 | 43.424 | 24.670 | 1.00 | 22.15 |
| ATOM | 3302 | CG | ASN | B | 620 | 25.289 | 44.538 | 25.691 | 1.00 | 22.79 |
| ATOM | 3303 | OD1 | ASN | B | 620 | 26.218 | 45.288 | 25.941 | 1.00 | 23.65 |
| ATOM | 3304 | ND2 | ASN | B | 620 | 24.110 | 44.632 | 26.292 | 1.00 | 22.68 |
| ATOM | 3305 | C | ASN | B | 620 | 26.857 | 42.524 | 22.806 | 1.00 | 18.58 |
| ATOM | 3306 | O | ASN | B | 620 | 27.787 | 41.809 | 23.178 | 1.00 | 19.87 |
| ATOM | 3307 | N | THR | B | 621 | 26.134 | 42.262 | 21.723 | 1.00 | 19.05 |
| ATOM | 3308 | CA | THR | B | 621 | 26.438 | 41.099 | 20.903 | 1.00 | 18.12 |
| ATOM | 3309 | CB | THR | B | 621 | 25.352 | 40.874 | 19.823 | 1.00 | 17.52 |
| ATOM | 3310 | OG1 | THR | B | 621 | 24.095 | 40.640 | 20.462 | 1.00 | 18.11 |
| ATOM | 3311 | CG2 | THR | B | 621 | 25.694 | 39.657 | 18.953 | 1.00 | 18.01 |
| ATOM | 3312 | C | THR | B | 621 | 27.796 | 41.323 | 20.233 | 1.00 | 18.57 |
| ATOM | 3313 | O | THR | B | 621 | 28.601 | 40.398 | 20.134 | 1.00 | 19.61 |
| ATOM | 3314 | N | ALA | B | 622 | 28.046 | 42.550 | 19.785 | 1.00 | 19.36 |
| ATOM | 3315 | CA | ALA | B | 622 | 29.310 | 42.896 | 19.140 | 1.00 | 18.22 |
| ATOM | 3316 | CB | ALA | B | 622 | 29.221 | 44.288 | 18.511 | 1.00 | 22.38 |
| ATOM | 3317 | C | ALA | B | 622 | 30.452 | 42.843 | 20.152 | 1.00 | 20.92 |
| ATOM | 3318 | O | ALA | B | 622 | 31.555 | 42.407 | 19.829 | 1.00 | 18.90 |
| ATOM | 3319 | N | GLN | B | 623 | 30.192 | 43.285 | 21.381 | 1.00 | 17.97 |
| ATOM | 3320 | CA | GLN | B | 623 | 31.219 | 43.257 | 22.420 | 1.00 | 19.34 |
| ATOM | 3321 | CB | GLN | B | 623 | 30.709 | 43.970 | 23.677 | 1.00 | 20.66 |
| ATOM | 3322 | CG | GLN | B | 623 | 31.720 | 44.081 | 24.823 | 1.00 | 21.04 |
| ATOM | 3323 | CD | GLN | B | 623 | 31.756 | 42.848 | 25.704 | 1.00 | 21.30 |
| ATOM | 3324 | OE1 | GLN | B | 623 | 30.712 | 42.346 | 26.134 | 1.00 | 22.15 |
| ATOM | 3325 | NE2 | GLN | B | 623 | 32.956 | 42.362 | 25.995 | 1.00 | 21.86 |
| ATOM | 3326 | C | GLN | B | 623 | 31.600 | 41.811 | 22.739 | 1.00 | 18.06 |
| ATOM | 3327 | O | GLN | B | 623 | 32.776 | 41.499 | 22.963 | 1.00 | 19.37 |
| ATOM | 3328 | N | CYS | B | 624 | 30.609 | 40.924 | 22.755 | 1.00 | 18.07 |
| ATOM | 3329 | CA | CYS | B | 624 | 30.880 | 39.519 | 23.027 | 1.00 | 17.94 |
| ATOM | 3330 | CB | CYS | B | 624 | 29.585 | 38.724 | 23.171 | 1.00 | 21.24 |
| ATOM | 3331 | SG | CYS | B | 624 | 29.857 | 37.007 | 23.684 | 1.00 | 20.09 |
| ATOM | 3332 | C | CYS | B | 624 | 31.695 | 38.961 | 21.866 | 1.00 | 19.06 |
| ATOM | 3333 | O | CYS | B | 624 | 32.615 | 38.158 | 22.067 | 1.00 | 18.98 |
| ATOM | 3334 | N | MET | B | 625 | 31.371 | 39.400 | 20.653 | 1.00 | 18.53 |
| ATOM | 3335 | CA | MET | B | 625 | 32.102 | 38.937 | 19.470 | 1.00 | 18.30 |
| ATOM | 3336 | CB | MET | B | 625 | 31.491 | 39.521 | 18.195 | 1.00 | 19.41 |
| ATOM | 3337 | CG | MET | B | 625 | 32.164 | 39.070 | 16.897 | 1.00 | 19.09 |
| ATOM | 3338 | SD | MET | B | 625 | 32.084 | 37.274 | 16.604 | 1.00 | 16.39 |
| ATOM | 3339 | CE | MET | B | 625 | 30.324 | 37.033 | 16.399 | 1.00 | 18.22 |
| ATOM | 3340 | C | MET | B | 625 | 33.555 | 39.393 | 19.606 | 1.00 | 18.55 |
| ATOM | 3341 | O | MET | B | 625 | 34.483 | 38.640 | 19.332 | 1.00 | 19.67 |
| ATOM | 3342 | N | PHE | B | 626 | 33.749 | 40.636 | 20.036 | 1.00 | 19.00 |
| ATOM | 3343 | CA | PHE | B | 626 | 35.101 | 41.156 | 20.225 | 1.00 | 18.99 |
| ATOM | 3344 | CB | PHE | B | 626 | 35.063 | 42.625 | 20.649 | 1.00 | 19.58 |
| ATOM | 3345 | CG | PHE | B | 626 | 36.421 | 43.211 | 20.909 | 1.00 | 19.51 |
| ATOM | 3346 | CD1 | PHE | B | 626 | 36.965 | 43.200 | 22.190 | 1.00 | 21.50 |
| ATOM | 3347 | CD2 | PHE | B | 626 | 37.162 | 43.759 | 19.868 | 1.00 | 20.23 |
| ATOM | 3348 | CE1 | PHE | B | 626 | 38.234 | 43.731 | 22.433 | 1.00 | 19.73 |
| ATOM | 3349 | CE2 | PHE | B | 626 | 38.433 | 44.292 | 20.095 | 1.00 | 20.70 |
| ATOM | 3350 | CZ | PHE | B | 626 | 38.971 | 44.277 | 21.384 | 1.00 | 20.75 |
| ATOM | 3351 | C | PHE | B | 626 | 35.851 | 40.352 | 21.279 | 1.00 | 20.36 |
| ATOM | 3352 | O | PHE | B | 626 | 36.993 | 39.951 | 21.066 | 1.00 | 19.87 |
| ATOM | 3353 | N | ALA | B | 627 | 35.211 | 40.117 | 22.420 | 1.00 | 20.43 |
| ATOM | 3354 | CA | ALA | B | 627 | 35.853 | 39.367 | 23.490 | 1.00 | 21.24 |
| ATOM | 3355 | CB | ALA | B | 627 | 34.950 | 39.322 | 24.713 | 1.00 | 23.11 |
| ATOM | 3356 | C | ALA | B | 627 | 36.192 | 37.949 | 23.033 | 1.00 | 20.95 |
| ATOM | 3357 | O | ALA | B | 627 | 37.274 | 37.433 | 23.329 | 1.00 | 21.81 |
| ATOM | 3358 | N | ALA | B | 628 | 35.267 | 37.327 | 22.305 | 1.00 | 19.64 |
| ATOM | 3359 | CA | ALA | B | 628 | 35.474 | 35.970 | 21.808 | 1.00 | 18.50 |
| ATOM | 3360 | CB | ALA | B | 628 | 34.194 | 35.453 | 21.156 | 1.00 | 21.27 |
| ATOM | 3361 | C | ALA | B | 628 | 36.619 | 35.901 | 20.808 | 1.00 | 18.67 |
| ATOM | 3362 | O | ALA | B | 628 | 37.415 | 34.958 | 20.827 | 1.00 | 21.28 |
| ATOM | 3363 | N | LEU | B | 629 | 36.695 | 36.890 | 19.921 | 1.00 | 19.77 |
| ATOM | 3364 | CA | LEU | B | 629 | 37.748 | 36.926 | 18.911 | 1.00 | 20.08 |
| ATOM | 3365 | CB | LEU | B | 629 | 37.449 | 38.026 | 17.885 | 1.00 | 20.64 |
| ATOM | 3366 | CG | LEU | B | 629 | 36.265 | 37.740 | 16.957 | 1.00 | 20.56 |
| ATOM | 3367 | CD1 | LEU | B | 629 | 35.933 | 38.980 | 16.138 | 1.00 | 20.95 |
| ATOM | 3368 | CD2 | LEU | B | 629 | 36.612 | 36.556 | 16.048 | 1.00 | 21.88 |
| ATOM | 3369 | C | LEU | B | 629 | 39.097 | 37.175 | 19.565 | 1.00 | 20.23 |
| ATOM | 3370 | O | LEU | B | 629 | 40.083 | 36.505 | 19.254 | 1.00 | 20.58 |
| ATOM | 3371 | N | LYS | B | 630 | 39.131 | 38.142 | 20.478 | 1.00 | 22.49 |
| ATOM | 3372 | CA | LYS | B | 630 | 40.354 | 38.517 | 21.184 | 1.00 | 24.06 |
| ATOM | 3373 | CB | LYS | B | 630 | 40.218 | 39.936 | 21.730 | 1.00 | 29.47 |
| ATOM | 3374 | CG | LYS | B | 630 | 40.467 | 41.003 | 20.712 | 1.00 | 30.15 |
| ATOM | 3375 | CD | LYS | B | 630 | 41.918 | 40.980 | 20.304 | 1.00 | 30.05 |
| ATOM | 3376 | CE | LYS | B | 630 | 42.193 | 42.037 | 19.271 | 1.00 | 29.88 |
| ATOM | 3377 | NZ | LYS | B | 630 | 43.597 | 41.963 | 18.828 | 1.00 | 29.81 |
| ATOM | 3378 | C | LYS | B | 630 | 40.727 | 37.597 | 22.331 | 1.00 | 24.07 |
| ATOM | 3379 | O | LYS | B | 630 | 41.585 | 36.724 | 22.197 | 1.00 | 23.83 |
| ATOM | 3380 | N | ALA | B | 631 | 40.091 | 37.815 | 23.475 | 1.00 | 24.13 |
| ATOM | 3381 | CA | ALA | B | 631 | 40.368 | 37.020 | 24.659 | 1.00 | 24.70 |
| ATOM | 3382 | CB | ALA | B | 631 | 39.527 | 37.516 | 25.828 | 1.00 | 25.17 |
| ATOM | 3383 | C | ALA | B | 631 | 40.121 | 35.534 | 24.437 | 1.00 | 25.41 |
| ATOM | 3384 | O | ALA | B | 631 | 40.882 | 34.700 | 24.922 | 1.00 | 25.73 |
| ATOM | 3385 | N | GLY | B | 632 | 39.060 | 35.203 | 23.705 | 1.00 | 24.21 |
| ATOM | 3386 | CA | GLY | B | 632 | 38.743 | 33.804 | 23.468 | 1.00 | 23.42 |
| ATOM | 3387 | C | GLY | B | 632 | 39.586 | 33.166 | 22.380 | 1.00 | 22.70 |
| ATOM | 3388 | O | GLY | B | 632 | 39.440 | 31.978 | 22.090 | 1.00 | 24.46 |
| ATOM | 3389 | N | LYS | B | 633 | 40.456 | 33.968 | 21.775 | 1.00 | 23.53 |
| ATOM | 3390 | CA | LYS | B | 633 | 41.357 | 33.531 | 20.717 | 1.00 | 24.70 |
| ATOM | 3391 | CB | LYS | B | 633 | 42.419 | 32.592 | 21.292 | 1.00 | 32.80 |
| ATOM | 3392 | CG | LYS | B | 633 | 43.316 | 33.272 | 22.310 | 1.00 | 33.19 |
| ATOM | 3393 | CD | LYS | B | 633 | 44.524 | 32.424 | 22.662 | 1.00 | 33.59 |
| ATOM | 3394 | CE | LYS | B | 633 | 45.416 | 33.167 | 23.643 | 1.00 | 33.78 |
| ATOM | 3395 | NZ | LYS | B | 633 | 45.749 | 34.527 | 23.130 | 1.00 | 34.10 |
| ATOM | 3396 | C | LYS | B | 633 | 40.709 | 32.888 | 19.499 | 1.00 | 24.23 |
| ATOM | 3397 | O | LYS | B | 633 | 41.243 | 31.935 | 18.928 | 1.00 | 24.88 |
| ATOM | 3398 | N | ILE | B | 634 | 39.565 | 33.411 | 19.079 | 1.00 | 22.51 |
| ATOM | 3399 | CA | ILE | B | 634 | 38.911 | 32.861 | 17.907 | 1.00 | 20.76 |
| ATOM | 3400 | CB | ILE | B | 634 | 37.376 | 32.982 | 18.036 | 1.00 | 22.93 |
| ATOM | 3401 | CG2 | ILE | B | 634 | 36.694 | 32.531 | 16.750 | 1.00 | 23.15 |
| ATOM | 3402 | CG1 | ILE | B | 634 | 36.907 | 32.122 | 19.218 | 1.00 | 22.60 |
| ATOM | 3403 | CD1 | ILE | B | 634 | 35.410 | 32.116 | 19.444 | 1.00 | 24.49 |
| ATOM | 3404 | C | ILE | B | 634 | 39.424 | 33.612 | 16.680 | 1.00 | 21.02 |
| ATOM | 3405 | O | ILE | B | 634 | 39.394 | 33.109 | 15.559 | 1.00 | 21.18 |
| ATOM | 3406 | N | GLN | B | 635 | 39.920 | 34.821 | 16.911 | 1.00 | 20.73 |
| ATOM | 3407 | CA | GLN | B | 635 | 40.453 | 35.656 | 15.848 | 1.00 | 21.87 |
| ATOM | 3408 | CB | GLN | B | 635 | 41.023 | 36.933 | 16.464 | 1.00 | 28.05 |
| ATOM | 3409 | CG | GLN | B | 635 | 41.703 | 37.868 | 15.503 | 1.00 | 29.39 |
| ATOM | 3410 | CD | GLN | B | 635 | 42.362 | 39.034 | 16.217 | 1.00 | 27.87 |
| ATOM | 3411 | OE1 | GLN | B | 635 | 43.020 | 39.856 | 15.596 | 1.00 | 29.76 |
| ATOM | 3412 | NE2 | GLN | B | 635 | 42.178 | 39.110 | 17.532 | 1.00 | 27.16 |
| ATOM | 3413 | C | GLN | B | 635 | 41.543 | 34.904 | 15.089 | 1.00 | 22.14 |
| ATOM | 3414 | O | GLN | B | 635 | 41.600 | 34.934 | 13.858 | 1.00 | 21.83 |
| ATOM | 3415 | N | ASN | B | 636 | 42.387 | 34.224 | 15.854 | 1.00 | 23.53 |
| ATOM | 3416 | CA | ASN | B | 636 | 43.504 | 33.447 | 15.335 | 1.00 | 24.32 |
| ATOM | 3417 | CB | ASN | B | 636 | 44.216 | 32.735 | 16.486 | 1.00 | 22.54 |
| ATOM | 3418 | CG | ASN | B | 636 | 44.607 | 33.675 | 17.606 | 1.00 | 22.42 |
| ATOM | 3419 | OD1 | ASN | B | 636 | 45.788 | 33.821 | 17.921 | 1.00 | 23.23 |
| ATOM | 3420 | ND2 | ASN | B | 636 | 43.620 | 34.307 | 18.221 | 1.00 | 21.19 |
| ATOM | 3421 | C | ASN | B | 636 | 43.089 | 32.390 | 14.325 | 1.00 | 24.28 |
| ATOM | 3422 | O | ASN | B | 636 | 43.868 | 32.022 | 13.443 | 1.00 | 24.97 |
| ATOM | 3423 | N | LYS | B | 637 | 41.864 | 31.899 | 14.463 | 1.00 | 22.71 |
| ATOM | 3424 | CA | LYS | B | 637 | 41.355 | 30.842 | 13.597 | 1.00 | 22.76 |
| ATOM | 3425 | CB | LYS | B | 637 | 40.417 | 29.948 | 14.409 | 1.00 | 25.00 |
| ATOM | 3426 | CG | LYS | B | 637 | 41.028 | 29.410 | 15.688 | 1.00 | 25.88 |
| ATOM | 3427 | CD | LYS | B | 637 | 39.983 | 28.674 | 16.515 | 1.00 | 24.87 |
| ATOM | 3428 | CE | LYS | B | 637 | 40.570 | 28.186 | 17.820 | 1.00 | 26.38 |
| ATOM | 3429 | NZ | LYS | B | 637 | 41.716 | 27.261 | 17.598 | 1.00 | 26.67 |
| ATOM | 3430 | C | LYS | B | 637 | 40.630 | 31.305 | 12.341 | 1.00 | 22.83 |
| ATOM | 3431 | O | LYS | B | 637 | 40.289 | 30.488 | 11.483 | 1.00 | 22.28 |
| ATOM | 3432 | N | LEU | B | 638 | 40.407 | 32.610 | 12.218 | 1.00 | 21.88 |
| ATOM | 3433 | CA | LEU | B | 638 | 39.669 | 33.131 | 11.081 | 1.00 | 22.21 |
| ATOM | 3434 | CB | LEU | B | 638 | 38.421 | 33.855 | 11.601 | 1.00 | 18.60 |
| ATOM | 3435 | CG | LEU | B | 638 | 37.598 | 33.093 | 12.650 | 1.00 | 20.22 |
| ATOM | 3436 | CD1 | LEU | B | 638 | 36.507 | 34.001 | 13.212 | 1.00 | 18.99 |
| ATOM | 3437 | CD2 | LEU | B | 638 | 36.985 | 31.847 | 12.035 | 1.00 | 19.54 |
| ATOM | 3438 | C | LEU | B | 638 | 40.447 | 34.061 | 10.160 | 1.00 | 21.99 |
| ATOM | 3439 | O | LEU | B | 638 | 41.531 | 34.549 | 10.502 | 1.00 | 21.62 |
| ATOM | 3440 | N | THR | B | 639 | 39.878 | 34.299 | 8.982 | 1.00 | 19.43 |
| ATOM | 3441 | CA | THR | B | 639 | 40.474 | 35.188 | 7.998 | 1.00 | 20.58 |
| ATOM | 3442 | CB | THR | B | 639 | 39.900 | 34.937 | 6.593 | 1.00 | 24.74 |
| ATOM | 3443 | OG1 | THR | B | 639 | 38.501 | 35.257 | 6.581 | 1.00 | 23.76 |
| ATOM | 3444 | CG2 | THR | B | 639 | 40.075 | 33.472 | 6.195 | 1.00 | 24.89 |
| ATOM | 3445 | C | THR | B | 639 | 40.110 | 36.609 | 8.413 | 1.00 | 20.43 |
| ATOM | 3446 | O | THR | B | 639 | 39.185 | 36.802 | 9.203 | 1.00 | 20.14 |
| ATOM | 3447 | N | ASP | B | 640 | 40.828 | 37.594 | 7.884 | 1.00 | 22.11 |
| ATOM | 3448 | CA | ASP | B | 640 | 40.550 | 38.989 | 8.195 | 1.00 | 21.37 |
| ATOM | 3449 | CB | ASP | B | 640 | 41.576 | 39.908 | 7.528 | 1.00 | 24.51 |
| ATOM | 3450 | CG | ASP | B | 640 | 42.964 | 39.763 | 8.119 | 1.00 | 24.64 |
| ATOM | 3451 | OD1 | ASP | B | 640 | 43.900 | 40.381 | 7.575 | 1.00 | 23.86 |
| ATOM | 3452 | OD2 | ASP | B | 640 | 43.118 | 39.040 | 9.124 | 1.00 | 24.36 |
| ATOM | 3453 | C | ASP | B | 640 | 39.150 | 39.360 | 7.718 | 1.00 | 22.46 |
| ATOM | 3454 | O | ASP | B | 640 | 38.440 | 40.094 | 8.398 | 1.00 | 21.08 |
| ATOM | 3455 | N | LEU | B | 641 | 38.761 | 38.850 | 6.549 | 1.00 | 22.62 |
| ATOM | 3456 | CA | LEU | B | 641 | 37.436 | 39.127 | 5.988 | 1.00 | 22.81 |
| ATOM | 3457 | CB | LEU | B | 641 | 37.312 | 38.504 | 4.594 | 1.00 | 24.97 |
| ATOM | 3458 | CG | LEU | B | 641 | 37.449 | 39.409 | 3.365 | 1.00 | 27.28 |
| ATOM | 3459 | CD1 | LEU | B | 641 | 38.476 | 40.500 | 3.601 | 1.00 | 25.76 |
| ATOM | 3460 | CD2 | LEU | B | 641 | 37.793 | 38.557 | 2.152 | 1.00 | 25.23 |
| ATOM | 3461 | C | LEU | B | 641 | 36.338 | 38.574 | 6.888 | 1.00 | 22.25 |
| ATOM | 3462 | O | LEU | B | 641 | 35.295 | 39.204 | 7.086 | 1.00 | 21.31 |
| ATOM | 3463 | N | GLU | B | 642 | 36.561 | 37.379 | 7.417 | 1.00 | 20.49 |
| ATOM | 3464 | CA | GLU | B | 642 | 35.588 | 36.765 | 8.301 | 1.00 | 20.51 |
| ATOM | 3465 | CB | GLU | B | 642 | 36.005 | 35.327 | 8.615 | 1.00 | 24.37 |
| ATOM | 3466 | CG | GLU | B | 642 | 35.704 | 34.391 | 7.460 | 1.00 | 24.67 |
| ATOM | 3467 | CD | GLU | B | 642 | 36.405 | 33.053 | 7.574 | 1.00 | 24.64 |
| ATOM | 3468 | OE1 | GLU | B | 642 | 36.092 | 32.159 | 6.760 | 1.00 | 27.01 |
| ATOM | 3469 | OE2 | GLU | B | 642 | 37.267 | 32.893 | 8.461 | 1.00 | 24.86 |
| ATOM | 3470 | C | GLU | B | 642 | 35.427 | 37.579 | 9.581 | 1.00 | 20.92 |
| ATOM | 3471 | O | GLU | B | 642 | 34.309 | 37.764 | 10.069 | 1.00 | 20.75 |
| ATOM | 3472 | N | ILE | B | 643 | 36.542 | 38.076 | 10.108 | 1.00 | 21.11 |
| ATOM | 3473 | CA | ILE | B | 643 | 36.529 | 38.880 | 11.325 | 1.00 | 19.45 |
| ATOM | 3474 | CB | ILE | B | 643 | 37.973 | 39.195 | 11.771 | 1.00 | 22.34 |
| ATOM | 3475 | CG2 | ILE | B | 643 | 37.970 | 40.210 | 12.912 | 1.00 | 21.69 |
| ATOM | 3476 | CG1 | ILE | B | 643 | 38.652 | 37.895 | 12.217 | 1.00 | 20.78 |
| ATOM | 3477 | CD1 | ILE | B | 643 | 40.165 | 37.993 | 12.357 | 1.00 | 22.35 |
| ATOM | 3478 | C | ILE | B | 643 | 35.762 | 40.177 | 11.055 | 1.00 | 19.94 |
| ATOM | 3479 | O | ILE | B | 643 | 34.921 | 40.598 | 11.858 | 1.00 | 19.49 |
| ATOM | 3480 | N | LEU | B | 644 | 36.044 | 40.790 | 9.911 | 1.00 | 19.97 |
| ATOM | 3481 | CA | LEU | B | 644 | 35.385 | 42.027 | 9.502 | 1.00 | 19.99 |
| ATOM | 3482 | CB | LEU | B | 644 | 35.926 | 42.466 | 8.135 | 1.00 | 21.41 |
| ATOM | 3483 | CG | LEU | B | 644 | 35.277 | 43.650 | 7.415 | 1.00 | 21.06 |
| ATOM | 3484 | CD1 | LEU | B | 644 | 35.449 | 44.915 | 8.234 | 1.00 | 22.38 |
| ATOM | 3485 | CD2 | LEU | B | 644 | 35.921 | 43.809 | 6.042 | 1.00 | 21.65 |
| ATOM | 3486 | C | LEU | B | 644 | 33.877 | 41.817 | 9.409 | 1.00 | 21.45 |
| ATOM | 3487 | O | LEU | B | 644 | 33.093 | 42.583 | 9.970 | 1.00 | 18.41 |
| ATOM | 3488 | N | ALA | B | 645 | 33.485 | 40.770 | 8.691 | 1.00 | 19.49 |
| ATOM | 3489 | CA | ALA | B | 645 | 32.076 | 40.444 | 8.499 | 1.00 | 20.21 |
| ATOM | 3490 | CB | ALA | B | 645 | 31.940 | 39.266 | 7.539 | 1.00 | 18.61 |
| ATOM | 3491 | C | ALA | B | 645 | 31.371 | 40.125 | 9.806 | 1.00 | 20.78 |
| ATOM | 3492 | O | ALA | B | 645 | 30.229 | 40.535 | 10.009 | 1.00 | 19.80 |
| ATOM | 3493 | N | LEU | B | 646 | 32.047 | 39.395 | 10.688 | 1.00 | 17.92 |
| ATOM | 3494 | CA | LEU | B | 646 | 31.455 | 39.028 | 11.968 | 1.00 | 18.51 |
| ATOM | 3495 | CB | LEU | B | 646 | 32.381 | 38.084 | 12.735 | 1.00 | 21.14 |
| ATOM | 3496 | CG | LEU | B | 646 | 32.393 | 36.631 | 12.250 | 1.00 | 19.75 |
| ATOM | 3497 | CD1 | LEU | B | 646 | 33.560 | 35.903 | 12.894 | 1.00 | 20.35 |
| ATOM | 3498 | CD2 | LEU | B | 646 | 31.077 | 35.951 | 12.587 | 1.00 | 20.12 |
| ATOM | 3499 | C | LEU | B | 646 | 31.138 | 40.241 | 12.837 | 1.00 | 17.72 |
| ATOM | 3500 | O | LEU | B | 646 | 30.075 | 40.304 | 13.460 | 1.00 | 18.91 |
| ATOM | 3501 | N | LEU | B | 647 | 32.054 | 41.202 | 12.893 | 1.00 | 18.38 |
| ATOM | 3502 | CA | LEU | B | 647 | 31.815 | 42.387 | 13.710 | 1.00 | 18.99 |
| ATOM | 3503 | CB | LEU | B | 647 | 33.074 | 43.255 | 13.800 | 1.00 | 16.80 |
| ATOM | 3504 | CG | LEU | B | 647 | 32.940 | 44.461 | 14.734 | 1.00 | 18.47 |
| ATOM | 3505 | CD1 | LEU | B | 647 | 32.637 | 43.978 | 16.146 | 1.00 | 18.48 |
| ATOM | 3506 | CD2 | LEU | B | 647 | 34.221 | 45.278 | 14.707 | 1.00 | 18.50 |
| ATOM | 3507 | C | LEU | B | 647 | 30.666 | 43.201 | 13.128 | 1.00 | 18.90 |
| ATOM | 3508 | O | LEU | B | 647 | 29.776 | 43.646 | 13.861 | 1.00 | 18.98 |
| ATOM | 3509 | N | ILE | B | 648 | 30.689 | 43.399 | 11.813 | 1.00 | 18.80 |
| ATOM | 3510 | CA | ILE | B | 648 | 29.633 | 44.146 | 11.134 | 1.00 | 17.53 |
| ATOM | 3511 | CB | ILE | B | 648 | 29.948 | 44.298 | 9.620 | 1.00 | 17.43 |
| ATOM | 3512 | CG2 | ILE | B | 648 | 28.747 | 44.886 | 8.884 | 1.00 | 16.56 |
| ATOM | 3513 | CG1 | ILE | B | 648 | 31.189 | 45.181 | 9.439 | 1.00 | 16.47 |
| ATOM | 3514 | CD1 | ILE | B | 648 | 31.622 | 45.377 | 7.987 | 1.00 | 16.64 |
| ATOM | 3515 | C | ILE | B | 648 | 28.303 | 43.405 | 11.322 | 1.00 | 19.67 |
| ATOM | 3516 | O | ILE | B | 648 | 27.268 | 44.020 | 11.608 | 1.00 | 19.92 |
| ATOM | 3517 | N | ALA | B | 649 | 28.327 | 42.084 | 11.170 | 1.00 | 17.14 |
| ATOM | 3518 | CA | ALA | B | 649 | 27.105 | 41.297 | 11.335 | 1.00 | 19.06 |
| ATOM | 3519 | CB | ALA | B | 649 | 27.353 | 39.830 | 10.964 | 1.00 | 15.65 |
| ATOM | 3520 | C | ALA | B | 649 | 26.562 | 41.381 | 12.753 | 1.00 | 17.80 |
| ATOM | 3521 | O | ALA | B | 649 | 25.360 | 41.559 | 12.957 | 1.00 | 16.89 |
| ATOM | 3522 | N | ALA | B | 650 | 27.442 | 41.245 | 13.738 | 1.00 | 17.06 |
| ATOM | 3523 | CA | ALA | B | 650 | 27.016 | 41.316 | 15.128 | 1.00 | 16.09 |
| ATOM | 3524 | CB | ALA | B | 650 | 28.219 | 41.166 | 16.056 | 1.00 | 20.12 |
| ATOM | 3525 | C | ALA | B | 650 | 26.299 | 42.641 | 15.406 | 1.00 | 17.22 |
| ATOM | 3526 | O | ALA | B | 650 | 25.239 | 42.660 | 16.031 | 1.00 | 18.13 |
| ATOM | 3527 | N | LEU | B | 651 | 26.875 | 43.739 | 14.935 | 1.00 | 18.31 |
| ATOM | 3528 | CA | LEU | B | 651 | 26.295 | 45.065 | 15.145 | 1.00 | 18.19 |
| ATOM | 3529 | CB | LEU | B | 651 | 27.302 | 46.143 | 14.744 | 1.00 | 17.38 |
| ATOM | 3530 | CG | LEU | B | 651 | 28.532 | 46.318 | 15.633 | 1.00 | 17.82 |
| ATOM | 3531 | CD1 | LEU | B | 651 | 29.580 | 47.146 | 14.917 | 1.00 | 17.72 |
| ATOM | 3532 | CD2 | LEU | B | 651 | 28.108 | 46.993 | 16.939 | 1.00 | 16.75 |
| ATOM | 3533 | C | LEU | B | 651 | 25.010 | 45.305 | 14.363 | 1.00 | 19.12 |
| ATOM | 3534 | O | LEU | B | 651 | 24.145 | 46.073 | 14.792 | 1.00 | 19.03 |
| ATOM | 3535 | N | SER | B | 652 | 24.879 | 44.644 | 13.218 | 1.00 | 16.50 |
| ATOM | 3536 | CA | SER | B | 652 | 23.717 | 44.861 | 12.361 | 1.00 | 15.50 |
| ATOM | 3537 | CB | SER | B | 652 | 24.165 | 45.025 | 10.902 | 1.00 | 19.61 |
| ATOM | 3538 | OG | SER | B | 652 | 25.258 | 45.908 | 10.760 | 1.00 | 18.90 |
| ATOM | 3539 | C | SER | B | 652 | 22.667 | 43.764 | 12.374 | 1.00 | 17.20 |
| ATOM | 3540 | O | SER | B | 652 | 21.616 | 43.932 | 11.770 | 1.00 | 16.98 |
| ATOM | 3541 | N | HIS | B | 653 | 22.922 | 42.664 | 13.073 | 1.00 | 19.06 |
| ATOM | 3542 | CA | HIS | B | 653 | 22.001 | 41.533 | 12.998 | 1.00 | 19.69 |
| ATOM | 3543 | CB | HIS | B | 653 | 22.594 | 40.312 | 13.697 | 1.00 | 17.94 |
| ATOM | 3544 | CG | HIS | B | 653 | 22.332 | 40.270 | 15.165 | 1.00 | 17.54 |
| ATOM | 3545 | CD2 | HIS | B | 653 | 21.396 | 39.612 | 15.888 | 1.00 | 16.38 |
| ATOM | 3546 | ND1 | HIS | B | 653 | 23.064 | 41.010 | 16.066 | 1.00 | 17.02 |
| ATOM | 3547 | CE1 | HIS | B | 653 | 22.592 | 40.809 | 17.282 | 1.00 | 14.84 |
| ATOM | 3548 | NE2 | HIS | B | 653 | 21.582 | 39.965 | 17.202 | 1.00 | 19.75 |
| ATOM | 3549 | C | HIS | B | 653 | 20.548 | 41.689 | 13.430 | 1.00 | 20.16 |
| ATOM | 3550 | O | HIS | B | 653 | 19.734 | 40.833 | 13.106 | 1.00 | 18.45 |
| ATOM | 3551 | N | ASP | B | 654 | 20.206 | 42.762 | 14.143 | 1.00 | 15.51 |
| ATOM | 3552 | CA | ASP | B | 654 | 18.808 | 42.945 | 14.562 | 1.00 | 15.82 |
| ATOM | 3553 | CB | ASP | B | 654 | 18.680 | 42.848 | 16.082 | 1.00 | 17.70 |
| ATOM | 3554 | CG | ASP | B | 654 | 18.401 | 41.444 | 16.565 | 1.00 | 16.46 |
| ATOM | 3555 | OD1 | ASP | B | 654 | 17.724 | 40.681 | 15.845 | 1.00 | 17.13 |
| ATOM | 3556 | OD2 | ASP | B | 654 | 18.826 | 41.109 | 17.689 | 1.00 | 16.53 |
| ATOM | 3557 | C | ASP | B | 654 | 18.246 | 44.300 | 14.120 | 1.00 | 15.98 |
| ATOM | 3558 | O | ASP | B | 654 | 17.249 | 44.775 | 14.679 | 1.00 | 16.02 |
| ATOM | 3559 | N | LEU | B | 655 | 18.875 | 44.903 | 13.113 | 1.00 | 14.12 |
| ATOM | 3560 | CA | LEU | B | 655 | 18.474 | 46.212 | 12.585 | 1.00 | 16.45 |
| ATOM | 3561 | CB | LEU | B | 655 | 19.191 | 46.465 | 11.253 | 1.00 | 22.99 |
| ATOM | 3562 | CG | LEU | B | 655 | 20.250 | 47.557 | 11.043 | 1.00 | 25.93 |
| ATOM | 3563 | CD1 | LEU | B | 655 | 20.660 | 48.211 | 12.341 | 1.00 | 24.18 |
| ATOM | 3564 | CD2 | LEU | B | 655 | 21.435 | 46.946 | 10.315 | 1.00 | 23.72 |
| ATOM | 3565 | C | LEU | B | 655 | 16.968 | 46.355 | 12.374 | 1.00 | 15.99 |
| ATOM | 3566 | O | LEU | B | 655 | 16.332 | 45.488 | 11.777 | 1.00 | 16.81 |
| ATOM | 3567 | N | ASP | B | 656 | 16.403 | 47.452 | 12.877 | 1.00 | 18.46 |
| ATOM | 3568 | CA | ASP | B | 656 | 14.975 | 47.741 | 12.723 | 1.00 | 18.67 |
| ATOM | 3569 | CB | ASP | B | 656 | 14.676 | 48.025 | 11.243 | 1.00 | 19.71 |
| ATOM | 3570 | CG | ASP | B | 656 | 13.318 | 48.677 | 11.028 | 1.00 | 21.79 |
| ATOM | 3571 | OD1 | ASP | B | 656 | 12.968 | 49.591 | 11.803 | 1.00 | 19.47 |
| ATOM | 3572 | OD2 | ASP | B | 656 | 12.612 | 48.282 | 10.075 | 1.00 | 21.77 |
| ATOM | 3573 | C | ASP | B | 656 | 14.046 | 46.647 | 13.244 | 1.00 | 19.00 |
| ATOM | 3574 | O | ASP | B | 656 | 12.965 | 46.434 | 12.697 | 1.00 | 18.21 |
| ATOM | 3575 | N | HIS | B | 657 | 14.460 | 45.952 | 14.298 | 1.00 | 17.23 |
| ATOM | 3576 | CA | HIS | B | 657 | 13.631 | 44.899 | 14.864 | 1.00 | 16.33 |
| ATOM | 3577 | CB | HIS | B | 657 | 14.377 | 44.174 | 15.983 | 1.00 | 19.47 |
| ATOM | 3578 | CG | HIS | B | 657 | 13.705 | 42.915 | 16.419 | 1.00 | 18.64 |
| ATOM | 3579 | CD2 | HIS | B | 657 | 12.475 | 42.701 | 16.941 | 1.00 | 17.65 |
| ATOM | 3580 | ND1 | HIS | B | 657 | 14.300 | 41.673 | 16.318 | 1.00 | 19.33 |
| ATOM | 3581 | CE1 | HIS | B | 657 | 13.467 | 40.752 | 16.763 | 1.00 | 16.32 |
| ATOM | 3582 | NE2 | HIS | B | 657 | 12.352 | 41.349 | 17.147 | 1.00 | 20.76 |
| ATOM | 3583 | C | HIS | B | 657 | 12.352 | 45.546 | 15.409 | 1.00 | 17.90 |
| ATOM | 3584 | O | HIS | B | 657 | 12.419 | 46.529 | 16.151 | 1.00 | 17.75 |
| ATOM | 3585 | N | PRO | B | 658 | 11.176 | 45.011 | 15.034 | 1.00 | 19.30 |
| ATOM | 3586 | CD | PRO | B | 658 | 11.026 | 43.936 | 14.032 | 1.00 | 15.12 |
| ATOM | 3587 | CA | PRO | B | 658 | 9.863 | 45.519 | 15.463 | 1.00 | 20.23 |
| ATOM | 3588 | CB | PRO | B | 658 | 8.922 | 44.970 | 14.398 | 1.00 | 15.90 |
| ATOM | 3589 | CG | PRO | B | 658 | 9.539 | 43.612 | 14.093 | 1.00 | 14.22 |
| ATOM | 3590 | C | PRO | B | 658 | 9.422 | 45.146 | 16.877 | 1.00 | 19.73 |
| ATOM | 3591 | O | PRO | B | 658 | 8.411 | 45.650 | 17.371 | 1.00 | 20.21 |
| ATOM | 3592 | N | GLY | B | 659 | 10.160 | 44.258 | 17.530 | 1.00 | 20.02 |
| ATOM | 3593 | CA | GLY | B | 659 | 9.800 | 43.886 | 18.884 | 1.00 | 20.59 |
| ATOM | 3594 | C | GLY | B | 659 | 8.794 | 42.760 | 18.918 | 1.00 | 20.21 |
| ATOM | 3595 | O | GLY | B | 659 | 8.194 | 42.491 | 19.954 | 1.00 | 21.72 |
| ATOM | 3596 | N | VAL | B | 660 | 8.589 | 42.117 | 17.772 | 1.00 | 20.05 |
| ATOM | 3597 | CA | VAL | B | 660 | 7.685 | 40.980 | 17.675 | 1.00 | 19.03 |
| ATOM | 3598 | CB | VAL | B | 660 | 6.353 | 41.336 | 16.963 | 1.00 | 22.30 |
| ATOM | 3599 | CG1 | VAL | B | 660 | 5.551 | 42.293 | 17.825 | 1.00 | 23.15 |
| ATOM | 3600 | CG2 | VAL | B | 660 | 6.623 | 41.946 | 15.599 | 1.00 | 22.17 |
| ATOM | 3601 | C | VAL | B | 660 | 8.432 | 39.915 | 16.882 | 1.00 | 20.57 |
| ATOM | 3602 | O | VAL | B | 660 | 9.340 | 40.233 | 16.115 | 1.00 | 20.96 |
| ATOM | 3603 | N | SER | B | 661 | 8.043 | 38.660 | 17.068 | 1.00 | 18.27 |
| ATOM | 3604 | CA | SER | B | 661 | 8.707 | 37.525 | 16.413 | 1.00 | 19.30 |
| ATOM | 3605 | CB | SER | B | 661 | 8.450 | 36.256 | 17.217 | 1.00 | 23.22 |
| ATOM | 3606 | OG | SER | B | 661 | 7.108 | 35.823 | 17.058 | 1.00 | 22.03 |
| ATOM | 3607 | C | SER | B | 661 | 8.337 | 37.237 | 14.961 | 1.00 | 19.20 |
| ATOM | 3608 | O | SER | B | 661 | 7.410 | 37.814 | 14.402 | 1.00 | 16.38 |
| ATOM | 3609 | N | ASN | B | 662 | 9.080 | 36.311 | 14.360 | 1.00 | 18.48 |
| ATOM | 3610 | CA | ASN | B | 662 | 8.810 | 35.905 | 12.989 | 1.00 | 17.98 |
| ATOM | 3611 | CB | ASN | B | 662 | 9.832 | 34.868 | 12.521 | 1.00 | 19.15 |
| ATOM | 3612 | CG | ASN | B | 662 | 11.117 | 35.491 | 12.060 | 1.00 | 20.55 |
| ATOM | 3613 | OD1 | ASN | B | 662 | 11.113 | 36.584 | 11.504 | 1.00 | 18.07 |
| ATOM | 3614 | ND2 | ASN | B | 662 | 12.229 | 34.788 | 12.263 | 1.00 | 19.60 |
| ATOM | 3615 | C | ASN | B | 662 | 7.428 | 35.275 | 12.943 | 1.00 | 18.27 |
| ATOM | 3616 | O | ASN | B | 662 | 6.651 | 35.512 | 12.020 | 1.00 | 19.29 |
| ATOM | 3617 | N | GLN | B | 663 | 7.129 | 34.463 | 13.952 | 1.00 | 19.10 |
| ATOM | 3618 | CA | GLN | B | 663 | 5.845 | 33.793 | 14.020 | 1.00 | 19.98 |
| ATOM | 3619 | CB | GLN | B | 663 | 5.763 | 32.909 | 15.267 | 1.00 | 47.82 |
| ATOM | 3620 | CG | GLN | B | 663 | 6.544 | 31.609 | 15.160 | 1.00 | 48.95 |
| ATOM | 3621 | CD | GLN | B | 663 | 8.056 | 31.793 | 15.228 | 1.00 | 49.43 |
| ATOM | 3622 | OE1 | GLN | B | 663 | 8.814 | 30.853 | 14.978 | 1.00 | 49.36 |
| ATOM | 3623 | NE2 | GLN | B | 663 | 8.499 | 32.997 | 15.576 | 1.00 | 48.96 |
| ATOM | 3624 | C | GLN | B | 663 | 4.708 | 34.798 | 14.029 | 1.00 | 19.45 |
| ATOM | 3625 | O | GLN | B | 663 | 3.701 | 34.609 | 13.352 | 1.00 | 19.22 |
| ATOM | 3626 | N | PHE | B | 664 | 4.883 | 35.869 | 14.795 | 1.00 | 17.76 |
| ATOM | 3627 | CA | PHE | B | 664 | 3.866 | 36.909 | 14.888 | 1.00 | 18.71 |
| ATOM | 3628 | CB | PHE | B | 664 | 4.266 | 37.946 | 15.938 | 1.00 | 20.58 |
| ATOM | 3629 | CG | PHE | B | 664 | 3.280 | 39.077 | 16.084 | 1.00 | 20.67 |
| ATOM | 3630 | CD1 | PHE | B | 664 | 3.340 | 40.185 | 15.243 | 1.00 | 20.97 |
| ATOM | 3631 | CD2 | PHE | B | 664 | 2.291 | 39.031 | 17.064 | 1.00 | 20.35 |
| ATOM | 3632 | CE1 | PHE | B | 664 | 2.428 | 41.232 | 15.377 | 1.00 | 20.58 |
| ATOM | 3633 | CE2 | PHE | B | 664 | 1.377 | 40.074 | 17.203 | 1.00 | 21.94 |
| ATOM | 3634 | CZ | PHE | B | 664 | 1.448 | 41.174 | 16.357 | 1.00 | 21.03 |
| ATOM | 3635 | C | PHE | B | 664 | 3.691 | 37.571 | 13.525 | 1.00 | 17.64 |
| ATOM | 3636 | O | PHE | B | 664 | 2.574 | 37.781 | 13.071 | 1.00 | 17.39 |
| ATOM | 3637 | N | LEU | B | 665 | 4.801 | 37.883 | 12.865 | 1.00 | 18.84 |
| ATOM | 3638 | CA | LEU | B | 665 | 4.734 | 38.504 | 11.552 | 1.00 | 20.08 |
| ATOM | 3639 | CB | LEU | B | 665 | 6.146 | 38.866 | 11.078 | 1.00 | 18.16 |
| ATOM | 3640 | CG | LEU | B | 665 | 6.834 | 39.975 | 11.886 | 1.00 | 19.07 |
| ATOM | 3641 | CD1 | LEU | B | 665 | 8.288 | 40.092 | 11.490 | 1.00 | 19.47 |
| ATOM | 3642 | CD2 | LEU | B | 665 | 6.119 | 41.289 | 11.654 | 1.00 | 18.80 |
| ATOM | 3643 | C | LEU | B | 665 | 4.039 | 37.582 | 10.540 | 1.00 | 20.00 |
| ATOM | 3644 | O | LEU | B | 665 | 3.350 | 38.044 | 9.627 | 1.00 | 21.88 |
| ATOM | 3645 | N | ILE | B | 666 | 4.222 | 36.276 | 10.700 | 1.00 | 18.71 |
| ATOM | 3646 | CA | ILE | B | 666 | 3.593 | 35.317 | 9.806 | 1.00 | 19.12 |
| ATOM | 3647 | CB | ILE | B | 666 | 4.239 | 33.920 | 9.951 | 1.00 | 22.08 |
| ATOM | 3648 | CG2 | ILE | B | 666 | 3.423 | 32.879 | 9.199 | 1.00 | 21.74 |
| ATOM | 3649 | CG1 | ILE | B | 666 | 5.674 | 33.967 | 9.421 | 1.00 | 21.76 |
| ATOM | 3650 | CD1 | ILE | B | 666 | 6.456 | 32.684 | 9.619 | 1.00 | 21.29 |
| ATOM | 3651 | C | ILE | B | 666 | 2.104 | 35.218 | 10.119 | 1.00 | 18.58 |
| ATOM | 3652 | O | ILE | B | 666 | 1.274 | 35.193 | 9.211 | 1.00 | 20.21 |
| ATOM | 3653 | N | ASN | B | 667 | 1.773 | 35.183 | 11.408 | 1.00 | 21.92 |
| ATOM | 3654 | CA | ASN | B | 667 | 0.378 | 35.072 | 11.837 | 1.00 | 22.79 |
| ATOM | 3655 | CB | ASN | B | 667 | 0.295 | 34.756 | 13.334 | 1.00 | 23.85 |
| ATOM | 3656 | CG | ASN | B | 667 | 0.779 | 33.354 | 13.669 | 1.00 | 23.91 |
| ATOM | 3657 | OD1 | ASN | B | 667 | 0.712 | 32.444 | 12.843 | 1.00 | 24.94 |
| ATOM | 3658 | ND2 | ASN | B | 667 | 1.250 | 33.173 | 14.895 | 1.00 | 23.83 |
| ATOM | 3659 | C | ASN | B | 667 | −0.472 | 36.305 | 11.544 | 1.00 | 23.14 |
| ATOM | 3660 | O | ASN | B | 667 | −1.702 | 36.213 | 11.470 | 1.00 | 22.85 |
| ATOM | 3661 | N | THR | B | 668 | 0.169 | 37.459 | 11.392 | 1.00 | 24.81 |
| ATOM | 3662 | CA | THR | B | 668 | −0.566 | 38.677 | 11.086 | 1.00 | 25.96 |
| ATOM | 3663 | CB | THR | B | 668 | −0.019 | 39.894 | 11.882 | 1.00 | 21.17 |
| ATOM | 3664 | OG1 | THR | B | 668 | 1.404 | 39.984 | 11.714 | 1.00 | 23.05 |
| ATOM | 3665 | CG2 | THR | B | 668 | −0.338 | 39.753 | 13.362 | 1.00 | 22.09 |
| ATOM | 3666 | C | THR | B | 668 | −0.511 | 38.973 | 9.585 | 1.00 | 26.69 |
| ATOM | 3667 | O | THR | B | 668 | −0.961 | 40.022 | 9.139 | 1.00 | 27.70 |
| ATOM | 3668 | N | ASN | B | 669 | 0.026 | 38.030 | 8.815 | 1.00 | 26.73 |
| ATOM | 3669 | CA | ASN | B | 669 | 0.138 | 38.169 | 7.362 | 1.00 | 27.16 |
| ATOM | 3670 | CB | ASN | B | 669 | −1.255 | 38.135 | 6.726 | 1.00 | 36.60 |
| ATOM | 3671 | CG | ASN | B | 669 | −1.940 | 36.796 | 6.906 | 1.00 | 36.71 |
| ATOM | 3672 | OD1 | ASN | B | 669 | −1.482 | 35.775 | 6.387 | 1.00 | 36.69 |
| ATOM | 3673 | ND2 | ASN | B | 669 | −3.039 | 36.789 | 7.650 | 1.00 | 37.50 |
| ATOM | 3674 | C | ASN | B | 669 | 0.867 | 39.453 | 6.978 | 1.00 | 27.24 |
| ATOM | 3675 | O | ASN | B | 669 | 0.440 | 40.195 | 6.088 | 1.00 | 26.93 |
| ATOM | 3676 | N | SER | B | 670 | 1.981 | 39.696 | 7.659 | 1.00 | 23.11 |
| ATOM | 3677 | CA | SER | B | 670 | 2.802 | 40.876 | 7.435 | 1.00 | 22.67 |
| ATOM | 3678 | CB | SER | B | 670 | 3.949 | 40.898 | 8.448 | 1.00 | 34.05 |
| ATOM | 3679 | OG | SER | B | 670 | 4.874 | 41.925 | 8.154 | 1.00 | 35.41 |
| ATOM | 3680 | C | SER | B | 670 | 3.382 | 40.916 | 6.028 | 1.00 | 22.16 |
| ATOM | 3681 | O | SER | B | 670 | 3.580 | 39.880 | 5.388 | 1.00 | 22.16 |
| ATOM | 3682 | N | GLU | B | 671 | 3.662 | 42.125 | 5.548 | 1.00 | 21.76 |
| ATOM | 3683 | CA | GLU | B | 671 | 4.236 | 42.279 | 4.225 | 1.00 | 21.00 |
| ATOM | 3684 | CB | GLU | B | 671 | 4.388 | 43.757 | 3.872 | 1.00 | 26.81 |
| ATOM | 3685 | CG | GLU | B | 671 | 4.799 | 43.990 | 2.433 | 1.00 | 26.53 |
| ATOM | 3686 | CD | GLU | B | 671 | 4.963 | 45.461 | 2.099 | 1.00 | 27.67 |
| ATOM | 3687 | OE1 | GLU | B | 671 | 6.052 | 46.018 | 2.361 | 1.00 | 28.24 |
| ATOM | 3688 | OE2 | GLU | B | 671 | 3.995 | 46.062 | 1.584 | 1.00 | 26.44 |
| ATOM | 3689 | C | GLU | B | 671 | 5.607 | 41.617 | 4.228 | 1.00 | 20.25 |
| ATOM | 3690 | O | GLU | B | 671 | 5.996 | 40.962 | 3.271 | 1.00 | 20.37 |
| ATOM | 3691 | N | LEU | B | 672 | 6.332 | 41.799 | 5.323 | 1.00 | 20.82 |
| ATOM | 3692 | CA | LEU | B | 672 | 7.664 | 41.222 | 5.476 | 1.00 | 21.28 |
| ATOM | 3693 | CB | LEU | B | 672 | 8.189 | 41.544 | 6.872 | 1.00 | 29.67 |
| ATOM | 3694 | CG | LEU | B | 672 | 9.522 | 42.279 | 7.026 | 1.00 | 30.73 |
| ATOM | 3695 | CD1 | LEU | B | 672 | 9.670 | 43.391 | 6.003 | 1.00 | 30.03 |
| ATOM | 3696 | CD2 | LEU | B | 672 | 9.588 | 42.833 | 8.438 | 1.00 | 30.18 |
| ATOM | 3697 | C | LEU | B | 672 | 7.610 | 39.711 | 5.277 | 1.00 | 21.18 |
| ATOM | 3698 | O | LEU | B | 672 | 8.470 | 39.121 | 4.620 | 1.00 | 20.85 |
| ATOM | 3699 | N | ALA | B | 673 | 6.585 | 39.084 | 5.844 | 1.00 | 21.74 |
| ATOM | 3700 | CA | ALA | B | 673 | 6.435 | 37.635 | 5.726 | 1.00 | 21.93 |
| ATOM | 3701 | CB | ALA | B | 673 | 5.285 | 37.150 | 6.607 | 1.00 | 19.72 |
| ATOM | 3702 | C | ALA | B | 673 | 6.202 | 37.211 | 4.282 | 1.00 | 22.57 |
| ATOM | 3703 | O | ALA | B | 673 | 6.723 | 36.189 | 3.839 | 1.00 | 21.41 |
| ATOM | 3704 | N | LEU | B | 674 | 5.410 | 37.987 | 3.548 | 1.00 | 20.77 |
| ATOM | 3705 | CA | LEU | B | 674 | 5.151 | 37.664 | 2.153 | 1.00 | 21.89 |
| ATOM | 3706 | CB | LEU | B | 674 | 4.078 | 38.586 | 1.572 | 1.00 | 30.80 |
| ATOM | 3707 | CG | LEU | B | 674 | 3.816 | 38.413 | 0.069 | 1.00 | 31.55 |
| ATOM | 3708 | CD1 | LEU | B | 674 | 3.312 | 37.005 | −0.217 | 1.00 | 31.01 |
| ATOM | 3709 | CD2 | LEU | B | 674 | 2.799 | 39.449 | −0.391 | 1.00 | 30.99 |
| ATOM | 3710 | C | LEU | B | 674 | 6.431 | 37.811 | 1.336 | 1.00 | 22.16 |
| ATOM | 3711 | O | LEU | B | 674 | 6.775 | 36.929 | 0.552 | 1.00 | 21.85 |
| ATOM | 3712 | N | MET | B | 675 | 7.132 | 38.922 | 1.536 | 1.00 | 23.84 |
| ATOM | 3713 | CA | MET | B | 675 | 8.366 | 39.192 | 0.809 | 1.00 | 25.40 |
| ATOM | 3714 | CB | MET | B | 675 | 8.979 | 40.513 | 1.278 | 1.00 | 42.14 |
| ATOM | 3715 | CG | MET | B | 675 | 8.153 | 41.746 | 0.946 | 1.00 | 42.93 |
| ATOM | 3716 | SD | MET | B | 675 | 8.869 | 43.254 | 1.635 | 1.00 | 43.64 |
| ATOM | 3717 | CE | MET | B | 675 | 10.093 | 43.646 | 0.397 | 1.00 | 43.26 |
| ATOM | 3718 | C | MET | B | 675 | 9.397 | 38.080 | 0.973 | 1.00 | 24.69 |
| ATOM | 3719 | O | MET | B | 675 | 10.072 | 37.706 | 0.010 | 1.00 | 25.28 |
| ATOM | 3720 | N | TYR | B | 676 | 9.514 | 37.546 | 2.186 | 1.00 | 22.91 |
| ATOM | 3721 | CA | TYR | B | 676 | 10.503 | 36.506 | 2.449 | 1.00 | 23.58 |
| ATOM | 3722 | CB | TYR | B | 676 | 11.316 | 36.899 | 3.687 | 1.00 | 22.54 |
| ATOM | 3723 | CG | TYR | B | 676 | 11.987 | 38.248 | 3.527 | 1.00 | 23.06 |
| ATOM | 3724 | CD1 | TYR | B | 676 | 11.763 | 39.276 | 4.437 | 1.00 | 23.12 |
| ATOM | 3725 | CE1 | TYR | B | 676 | 12.338 | 40.539 | 4.259 | 1.00 | 22.13 |
| ATOM | 3726 | CD2 | TYR | B | 676 | 12.814 | 38.508 | 2.432 | 1.00 | 24.18 |
| ATOM | 3727 | CE2 | TYR | B | 676 | 13.400 | 39.762 | 2.247 | 1.00 | 22.56 |
| ATOM | 3728 | CZ | TYR | B | 676 | 13.155 | 40.772 | 3.161 | 1.00 | 23.95 |
| ATOM | 3729 | OH | TYR | B | 676 | 13.719 | 42.015 | 2.974 | 1.00 | 23.51 |
| ATOM | 3730 | C | TYR | B | 676 | 9.973 | 35.074 | 2.578 | 1.00 | 22.94 |
| ATOM | 3731 | O | TYR | B | 676 | 10.640 | 34.204 | 3.144 | 1.00 | 23.18 |
| ATOM | 3732 | N | ASN | B | 677 | 8.781 | 34.840 | 2.034 | 1.00 | 23.86 |
| ATOM | 3733 | CA | ASN | B | 677 | 8.137 | 33.524 | 2.034 | 1.00 | 25.01 |
| ATOM | 3734 | CB | ASN | B | 677 | 8.760 | 32.633 | 0.948 | 1.00 | 56.05 |
| ATOM | 3735 | CG | ASN | B | 677 | 10.243 | 32.381 | 1.169 | 1.00 | 57.14 |
| ATOM | 3736 | OD1 | ASN | B | 677 | 10.642 | 31.796 | 2.177 | 1.00 | 57.41 |
| ATOM | 3737 | ND2 | ASN | B | 677 | 11.069 | 32.819 | 0.222 | 1.00 | 57.47 |
| ATOM | 3738 | C | ASN | B | 677 | 8.139 | 32.783 | 3.373 | 1.00 | 24.18 |
| ATOM | 3739 | O | ASN | B | 677 | 8.385 | 31.575 | 3.430 | 1.00 | 22.27 |
| ATOM | 3740 | N | ASP | B | 678 | 7.850 | 33.519 | 4.442 | 1.00 | 20.57 |
| ATOM | 3741 | CA | ASP | B | 678 | 7.783 | 32.970 | 5.794 | 1.00 | 20.56 |
| ATOM | 3742 | CB | ASP | B | 678 | 6.692 | 31.895 | 5.876 | 1.00 | 24.90 |
| ATOM | 3743 | CG | ASP | B | 678 | 5.310 | 32.442 | 5.609 | 1.00 | 26.44 |
| ATOM | 3744 | OD1 | ASP | B | 678 | 5.139 | 33.676 | 5.661 | 1.00 | 24.56 |
| ATOM | 3745 | OD2 | ASP | B | 678 | 4.386 | 31.632 | 5.359 | 1.00 | 26.91 |
| ATOM | 3746 | C | ASP | B | 678 | 9.074 | 32.390 | 6.358 | 1.00 | 19.72 |
| ATOM | 3747 | O | ASP | B | 678 | 9.067 | 31.843 | 7.464 | 1.00 | 21.82 |
| ATOM | 3748 | N | GLU | B | 679 | 10.175 | 32.513 | 5.625 | 1.00 | 18.31 |
| ATOM | 3749 | CA | GLU | B | 679 | 11.452 | 31.964 | 6.086 | 1.00 | 18.54 |
| ATOM | 3750 | CB | GLU | B | 679 | 12.148 | 31.211 | 4.955 | 1.00 | 42.65 |
| ATOM | 3751 | CG | GLU | B | 679 | 11.459 | 29.942 | 4.518 | 1.00 | 43.73 |
| ATOM | 3752 | CD | GLU | B | 679 | 12.276 | 29.188 | 3.492 | 1.00 | 44.02 |
| ATOM | 3753 | OE1 | GLU | B | 679 | 12.480 | 29.723 | 2.381 | 1.00 | 44.05 |
| ATOM | 3754 | OE2 | GLU | B | 679 | 12.723 | 28.066 | 3.803 | 1.00 | 44.29 |
| ATOM | 3755 | C | GLU | B | 679 | 12.407 | 33.021 | 6.611 | 1.00 | 18.73 |
| ATOM | 3756 | O | GLU | B | 679 | 12.768 | 33.939 | 5.875 | 1.00 | 19.73 |
| ATOM | 3757 | N | SER | B | 680 | 12.832 | 32.866 | 7.867 | 1.00 | 18.88 |
| ATOM | 3758 | CA | SER | B | 680 | 13.758 | 33.798 | 8.506 | 1.00 | 16.49 |
| ATOM | 3759 | CB | SER | B | 680 | 15.197 | 33.508 | 8.065 | 1.00 | 21.45 |
| ATOM | 3760 | OG | SER | B | 680 | 15.556 | 32.162 | 8.358 | 1.00 | 20.83 |
| ATOM | 3761 | C | SER | B | 680 | 13.383 | 35.226 | 8.127 | 1.00 | 16.21 |
| ATOM | 3762 | O | SER | B | 680 | 14.232 | 36.019 | 7.722 | 1.00 | 14.77 |
| ATOM | 3763 | N | VAL | B | 681 | 12.099 | 35.536 | 8.272 | 1.00 | 17.92 |
| ATOM | 3764 | CA | VAL | B | 681 | 11.558 | 36.838 | 7.904 | 1.00 | 16.01 |
| ATOM | 3765 | CB | VAL | B | 681 | 10.084 | 36.928 | 8.328 | 1.00 | 18.23 |
| ATOM | 3766 | CG1 | VAL | B | 681 | 9.501 | 38.295 | 7.956 | 1.00 | 18.53 |
| ATOM | 3767 | CG2 | VAL | B | 681 | 9.295 | 35.822 | 7.634 | 1.00 | 19.77 |
| ATOM | 3768 | C | VAL | B | 681 | 12.328 | 38.038 | 8.440 | 1.00 | 15.81 |
| ATOM | 3769 | O | VAL | B | 681 | 12.799 | 38.872 | 7.667 | 1.00 | 17.42 |
| ATOM | 3770 | N | LEU | B | 681A | 12.449 | 38.120 | 9.761 | 1.00 | 15.91 |
| ATOM | 3771 | CA | LEU | B | 681A | 13.164 | 39.216 | 10.410 | 1.00 | 17.15 |
| ATOM | 3772 | CB | LEU | B | 681A | 13.094 | 39.055 | 11.925 | 1.00 | 20.96 |
| ATOM | 3773 | CG | LEU | B | 681A | 11.729 | 39.310 | 12.559 | 1.00 | 24.02 |
| ATOM | 3774 | CD1 | LEU | B | 681A | 11.671 | 38.670 | 13.947 | 1.00 | 23.49 |
| ATOM | 3775 | CD2 | LEU | B | 681A | 11.500 | 40.805 | 12.619 | 1.00 | 23.37 |
| ATOM | 3776 | C | LEU | B | 681A | 14.626 | 39.293 | 9.997 | 1.00 | 17.72 |
| ATOM | 3777 | O | LEU | B | 681A | 15.151 | 40.367 | 9.733 | 1.00 | 16.37 |
| ATOM | 3778 | N | GLU | B | 682 | 15.281 | 38.141 | 9.936 | 1.00 | 16.35 |
| ATOM | 3779 | CA | GLU | B | 682 | 16.690 | 38.112 | 9.582 | 1.00 | 16.54 |
| ATOM | 3780 | CB | GLU | B | 682 | 17.226 | 36.693 | 9.797 | 1.00 | 17.48 |
| ATOM | 3781 | CG | GLU | B | 682 | 17.172 | 36.219 | 11.264 | 1.00 | 19.45 |
| ATOM | 3782 | CD | GLU | B | 682 | 15.765 | 35.939 | 11.783 | 1.00 | 17.96 |
| ATOM | 3783 | OE1 | GLU | B | 682 | 14.873 | 35.587 | 10.975 | 1.00 | 16.02 |
| ATOM | 3784 | OE2 | GLU | B | 682 | 15.553 | 36.043 | 13.015 | 1.00 | 18.67 |
| ATOM | 3785 | C | GLU | B | 682 | 16.957 | 38.616 | 8.162 | 1.00 | 16.65 |
| ATOM | 3786 | O | GLU | B | 682 | 17.956 | 39.307 | 7.908 | 1.00 | 17.06 |
| ATOM | 3787 | N | HIS | B | 683 | 16.068 | 38.286 | 7.233 | 1.00 | 16.83 |
| ATOM | 3788 | CA | HIS | B | 683 | 16.225 | 38.780 | 5.880 | 1.00 | 17.12 |
| ATOM | 3789 | CB | HIS | B | 683 | 15.193 | 38.144 | 4.953 | 1.00 | 21.77 |
| ATOM | 3790 | CG | HIS | B | 683 | 15.579 | 36.779 | 4.481 | 1.00 | 20.98 |
| ATOM | 3791 | CD2 | HIS | B | 683 | 15.140 | 35.552 | 4.845 | 1.00 | 21.60 |
| ATOM | 3792 | ND1 | HIS | B | 683 | 16.550 | 36.571 | 3.524 | 1.00 | 21.61 |
| ATOM | 3793 | CE1 | HIS | B | 683 | 16.690 | 35.274 | 3.317 | 1.00 | 20.39 |
| ATOM | 3794 | NE2 | HIS | B | 683 | 15.847 | 34.634 | 4.107 | 1.00 | 21.29 |
| ATOM | 3795 | C | HIS | B | 683 | 16.050 | 40.291 | 5.923 | 1.00 | 16.91 |
| ATOM | 3796 | O | HIS | B | 683 | 16.745 | 41.017 | 5.220 | 1.00 | 18.17 |
| ATOM | 3797 | N | HIS | B | 684 | 15.136 | 40.756 | 6.772 | 1.00 | 16.37 |
| ATOM | 3798 | CA | HIS | B | 684 | 14.900 | 42.194 | 6.906 | 1.00 | 18.67 |
| ATOM | 3799 | CB | HIS | B | 684 | 13.674 | 42.472 | 7.789 | 1.00 | 18.26 |
| ATOM | 3800 | CG | HIS | B | 684 | 13.376 | 43.934 | 7.962 | 1.00 | 19.15 |
| ATOM | 3801 | CD2 | HIS | B | 684 | 13.327 | 44.714 | 9.068 | 1.00 | 20.53 |
| ATOM | 3802 | ND1 | HIS | B | 684 | 13.124 | 44.769 | 6.896 | 1.00 | 20.23 |
| ATOM | 3803 | CE1 | HIS | B | 684 | 12.939 | 46.002 | 7.336 | 1.00 | 19.60 |
| ATOM | 3804 | NE2 | HIS | B | 684 | 13.056 | 45.996 | 8.650 | 1.00 | 19.94 |
| ATOM | 3805 | C | HIS | B | 684 | 16.131 | 42.858 | 7.516 | 1.00 | 18.24 |
| ATOM | 3806 | O | HIS | B | 684 | 16.556 | 43.927 | 7.066 | 1.00 | 19.27 |
| ATOM | 3807 | N | HIS | B | 685 | 16.706 | 42.228 | 8.537 | 1.00 | 18.19 |
| ATOM | 3808 | CA | HIS | B | 685 | 17.890 | 42.797 | 9.180 | 1.00 | 17.28 |
| ATOM | 3809 | CB | HIS | B | 685 | 18.349 | 41.957 | 10.376 | 1.00 | 17.43 |
| ATOM | 3810 | CG | HIS | B | 685 | 17.301 | 41.751 | 11.425 | 1.00 | 18.40 |
| ATOM | 3811 | CD2 | HIS | B | 685 | 16.971 | 40.654 | 12.148 | 1.00 | 18.57 |
| ATOM | 3812 | ND1 | HIS | B | 685 | 16.479 | 42.765 | 11.871 | 1.00 | 17.46 |
| ATOM | 3813 | CE1 | HIS | B | 685 | 15.689 | 42.299 | 12.823 | 1.00 | 17.20 |
| ATOM | 3814 | NE2 | HIS | B | 685 | 15.967 | 41.022 | 13.011 | 1.00 | 17.24 |
| ATOM | 3815 | C | HIS | B | 685 | 19.028 | 42.908 | 8.175 | 1.00 | 18.93 |
| ATOM | 3816 | O | HIS | B | 685 | 19.725 | 43.919 | 8.140 | 1.00 | 16.49 |
| ATOM | 3817 | N | PHE | B | 686 | 19.227 | 41.870 | 7.363 | 1.00 | 18.43 |
| ATOM | 3818 | CA | PHE | B | 686 | 20.289 | 41.922 | 6.363 | 1.00 | 19.77 |
| ATOM | 3819 | CB | PHE | B | 686 | 20.430 | 40.587 | 5.625 | 1.00 | 22.01 |
| ATOM | 3820 | CG | PHE | B | 686 | 21.424 | 40.635 | 4.501 | 1.00 | 22.74 |
| ATOM | 3821 | CD1 | PHE | B | 686 | 22.771 | 40.879 | 4.757 | 1.00 | 22.40 |
| ATOM | 3822 | CD2 | PHE | B | 686 | 21.008 | 40.509 | 3.185 | 1.00 | 22.19 |
| ATOM | 3823 | CE1 | PHE | B | 686 | 23.689 | 41.003 | 3.714 | 1.00 | 22.33 |
| ATOM | 3824 | CE2 | PHE | B | 686 | 21.915 | 40.631 | 2.139 | 1.00 | 23.22 |
| ATOM | 3825 | CZ | PHE | B | 686 | 23.259 | 40.882 | 2.406 | 1.00 | 22.45 |
| ATOM | 3826 | C | PHE | B | 686 | 20.017 | 43.025 | 5.345 | 1.00 | 19.33 |
| ATOM | 3827 | O | PHE | B | 686 | 20.924 | 43.747 | 4.938 | 1.00 | 19.34 |
| ATOM | 3828 | N | ASP | B | 687 | 18.762 | 43.149 | 4.927 | 1.00 | 20.18 |
| ATOM | 3829 | CA | ASP | B | 687 | 18.390 | 44.175 | 3.974 | 1.00 | 21.34 |
| ATOM | 3830 | CB | ASP | B | 687 | 16.886 | 44.130 | 3.728 | 1.00 | 29.63 |
| ATOM | 3831 | CG | ASP | B | 687 | 16.421 | 45.207 | 2.777 | 1.00 | 30.70 |
| ATOM | 3832 | OD1 | ASP | B | 687 | 16.880 | 45.210 | 1.616 | 1.00 | 31.13 |
| ATOM | 3833 | OD2 | ASP | B | 687 | 15.602 | 46.053 | 3.194 | 1.00 | 32.12 |
| ATOM | 3834 | C | ASP | B | 687 | 18.789 | 45.539 | 4.526 | 1.00 | 20.19 |
| ATOM | 3835 | O | ASP | B | 687 | 19.402 | 46.344 | 3.826 | 1.00 | 20.58 |
| ATOM | 3836 | N | GLN | B | 688 | 18.447 | 45.779 | 5.790 | 1.00 | 19.31 |
| ATOM | 3837 | CA | GLN | B | 688 | 18.773 | 47.044 | 6.447 | 1.00 | 20.14 |
| ATOM | 3838 | CB | GLN | B | 688 | 18.145 | 47.090 | 7.842 | 1.00 | 25.17 |
| ATOM | 3839 | CG | GLN | B | 688 | 16.621 | 47.113 | 7.823 | 1.00 | 26.26 |
| ATOM | 3840 | CD | GLN | B | 688 | 16.074 | 48.399 | 7.241 | 1.00 | 26.81 |
| ATOM | 3841 | OE1 | GLN | B | 688 | 16.218 | 49.471 | 7.835 | 1.00 | 28.04 |
| ATOM | 3842 | NE2 | GLN | B | 688 | 15.456 | 48.306 | 6.071 | 1.00 | 26.91 |
| ATOM | 3843 | C | GLN | B | 688 | 20.282 | 47.246 | 6.549 | 1.00 | 20.35 |
| ATOM | 3844 | O | GLN | B | 688 | 20.777 | 48.366 | 6.389 | 1.00 | 19.51 |
| ATOM | 3845 | N | CYS | B | 689 | 21.003 | 46.159 | 6.811 | 1.00 | 19.79 |
| ATOM | 3846 | CA | CYS | B | 689 | 22.457 | 46.199 | 6.924 | 1.00 | 20.08 |
| ATOM | 3847 | CB | CYS | B | 689 | 22.984 | 44.803 | 7.271 | 1.00 | 19.48 |
| ATOM | 3848 | SG | CYS | B | 689 | 24.795 | 44.663 | 7.336 | 1.00 | 17.52 |
| ATOM | 3849 | C | CYS | B | 689 | 23.085 | 46.679 | 5.617 | 1.00 | 19.76 |
| ATOM | 3850 | O | CYS | B | 689 | 23.949 | 47.563 | 5.605 | 1.00 | 19.63 |
| ATOM | 3851 | N | LEU | B | 690 | 22.655 | 46.072 | 4.517 | 1.00 | 19.56 |
| ATOM | 3852 | CA | LEU | B | 690 | 23.147 | 46.417 | 3.192 | 1.00 | 20.27 |
| ATOM | 3853 | CB | LEU | B | 690 | 22.516 | 45.489 | 2.154 | 1.00 | 27.59 |
| ATOM | 3854 | CG | LEU | B | 690 | 23.083 | 45.572 | 0.739 | 1.00 | 27.68 |
| ATOM | 3855 | CD1 | LEU | B | 690 | 24.542 | 45.123 | 0.746 | 1.00 | 28.21 |
| ATOM | 3856 | CD2 | LEU | B | 690 | 22.257 | 44.692 | −0.183 | 1.00 | 28.78 |
| ATOM | 3857 | C | LEU | B | 690 | 22.795 | 47.867 | 2.872 | 1.00 | 20.36 |
| ATOM | 3858 | O | LEU | B | 690 | 23.607 | 48.606 | 2.332 | 1.00 | 21.09 |
| ATOM | 3859 | N | MET | B | 691 | 21.571 | 48.264 | 3.206 | 1.00 | 21.14 |
| ATOM | 3860 | CA | MET | B | 691 | 21.120 | 49.630 | 2.961 | 1.00 | 22.00 |
| ATOM | 3861 | CB | MET | B | 691 | 19.730 | 49.839 | 3.568 | 1.00 | 37.31 |
| ATOM | 3862 | CG | MET | B | 691 | 19.151 | 51.226 | 3.340 | 1.00 | 37.86 |
| ATOM | 3863 | SD | MET | B | 691 | 17.539 | 51.412 | 4.127 | 1.00 | 38.97 |
| ATOM | 3864 | CE | MET | B | 691 | 18.030 | 51.835 | 5.809 | 1.00 | 38.00 |
| ATOM | 3865 | C | MET | B | 691 | 22.109 | 50.629 | 3.572 | 1.00 | 22.35 |
| ATOM | 3866 | O | MET | B | 691 | 22.552 | 51.559 | 2.902 | 1.00 | 22.19 |
| ATOM | 3867 | N | ILE | B | 692 | 22.455 | 50.420 | 4.839 | 1.00 | 21.40 |
| ATOM | 3868 | CA | ILE | B | 692 | 23.385 | 51.308 | 5.534 | 1.00 | 21.97 |
| ATOM | 3869 | CB | ILE | B | 692 | 23.522 | 50.930 | 7.029 | 1.00 | 25.90 |
| ATOM | 3870 | CG2 | ILE | B | 692 | 24.515 | 51.866 | 7.710 | 1.00 | 24.75 |
| ATOM | 3871 | CG1 | ILE | B | 692 | 22.159 | 51.011 | 7.727 | 1.00 | 26.02 |
| ATOM | 3872 | CD1 | ILE | B | 692 | 21.569 | 52.395 | 7.770 | 1.00 | 26.52 |
| ATOM | 3873 | C | ILE | B | 692 | 24.775 | 51.286 | 4.899 | 1.00 | 22.83 |
| ATOM | 3874 | O | ILE | B | 692 | 25.391 | 52.332 | 4.704 | 1.00 | 21.06 |
| ATOM | 3875 | N | LEU | B | 693 | 25.261 | 50.092 | 4.566 | 1.00 | 21.77 |
| ATOM | 3876 | CA | LEU | B | 693 | 26.581 | 49.951 | 3.951 | 1.00 | 23.08 |
| ATOM | 3877 | CB | LEU | B | 693 | 26.940 | 48.468 | 3.787 | 1.00 | 21.71 |
| ATOM | 3878 | CG | LEU | B | 693 | 27.301 | 47.701 | 5.060 | 1.00 | 21.98 |
| ATOM | 3879 | CD1 | LEU | B | 693 | 27.277 | 46.218 | 4.784 | 1.00 | 22.22 |
| ATOM | 3880 | CD2 | LEU | B | 693 | 28.677 | 48.144 | 5.553 | 1.00 | 22.27 |
| ATOM | 3881 | C | LEU | B | 693 | 26.664 | 50.635 | 2.595 | 1.00 | 23.02 |
| ATOM | 3882 | O | LEU | B | 693 | 27.752 | 51.009 | 2.153 | 1.00 | 24.07 |
| ATOM | 3883 | N | ASN | B | 694 | 25.519 | 50.794 | 1.934 | 1.00 | 24.41 |
| ATOM | 3884 | CA | ASN | B | 694 | 25.478 | 51.437 | 0.626 | 1.00 | 24.18 |
| ATOM | 3885 | CB | ASN | B | 694 | 24.448 | 50.760 | −0.285 | 1.00 | 28.06 |
| ATOM | 3886 | CG | ASN | B | 694 | 24.886 | 49.387 | −0.756 | 1.00 | 28.56 |
| ATOM | 3887 | OD1 | ASN | B | 694 | 26.040 | 49.184 | −1.122 | 1.00 | 29.03 |
| ATOM | 3888 | ND2 | ASN | B | 694 | 23.953 | 48.439 | −0.769 | 1.00 | 28.87 |
| ATOM | 3889 | C | ASN | B | 694 | 25.149 | 52.922 | 0.715 | 1.00 | 25.26 |
| ATOM | 3890 | O | ASN | B | 694 | 25.185 | 53.625 | −0.295 | 1.00 | 25.49 |
| ATOM | 3891 | N | SER | B | 695 | 24.830 | 53.392 | 1.917 | 1.00 | 24.51 |
| ATOM | 3892 | CA | SER | B | 695 | 24.483 | 54.796 | 2.124 | 1.00 | 26.67 |
| ATOM | 3893 | CB | SER | B | 695 | 23.842 | 54.991 | 3.497 | 1.00 | 38.96 |
| ATOM | 3894 | OG | SER | B | 695 | 22.544 | 54.433 | 3.532 | 1.00 | 40.66 |
| ATOM | 3895 | C | SER | B | 695 | 25.679 | 55.724 | 1.999 | 1.00 | 26.23 |
| ATOM | 3896 | O | SER | B | 695 | 26.750 | 55.447 | 2.534 | 1.00 | 25.50 |
| ATOM | 3897 | N | PRO | B | 696 | 25.503 | 56.854 | 1.298 | 1.00 | 30.83 |
| ATOM | 3898 | CD | PRO | B | 696 | 24.242 | 57.397 | 0.766 | 1.00 | 35.10 |
| ATOM | 3899 | CA | PRO | B | 696 | 26.600 | 57.810 | 1.125 | 1.00 | 31.36 |
| ATOM | 3900 | CB | PRO | B | 696 | 25.936 | 58.959 | 0.362 | 1.00 | 35.40 |
| ATOM | 3901 | CG | PRO | B | 696 | 24.499 | 58.882 | 0.813 | 1.00 | 35.60 |
| ATOM | 3902 | C | PRO | B | 696 | 27.205 | 58.251 | 2.461 | 1.00 | 31.40 |
| ATOM | 3903 | O | PRO | B | 696 | 26.480 | 58.569 | 3.406 | 1.00 | 31.21 |
| ATOM | 3904 | N | GLY | B | 697 | 28.535 | 58.250 | 2.528 | 1.00 | 27.19 |
| ATOM | 3905 | CA | GLY | B | 697 | 29.227 | 58.653 | 3.741 | 1.00 | 26.21 |
| ATOM | 3906 | C | GLY | B | 697 | 29.268 | 57.587 | 4.821 | 1.00 | 25.99 |
| ATOM | 3907 | O | GLY | B | 697 | 29.813 | 57.811 | 5.901 | 1.00 | 25.64 |
| ATOM | 3908 | N | ASN | B | 698 | 28.698 | 56.422 | 4.532 | 1.00 | 25.20 |
| ATOM | 3909 | CA | ASN | B | 698 | 28.661 | 55.328 | 5.498 | 1.00 | 24.25 |
| ATOM | 3910 | CB | ASN | B | 698 | 27.215 | 54.970 | 5.867 | 1.00 | 26.49 |
| ATOM | 3911 | CG | ASN | B | 698 | 26.516 | 56.050 | 6.651 | 1.00 | 26.36 |
| ATOM | 3912 | OD1 | ASN | B | 698 | 26.081 | 57.067 | 6.101 | 1.00 | 28.19 |
| ATOM | 3913 | ND2 | ASN | B | 698 | 26.399 | 55.834 | 7.948 | 1.00 | 26.66 |
| ATOM | 3914 | C | ASN | B | 698 | 29.302 | 54.053 | 4.967 | 1.00 | 25.12 |
| ATOM | 3915 | O | ASN | B | 698 | 29.271 | 53.025 | 5.639 | 1.00 | 23.91 |
| ATOM | 3916 | N | GLN | B | 699 | 29.876 | 54.114 | 3.771 | 1.00 | 24.45 |
| ATOM | 3917 | CA | GLN | B | 699 | 30.456 | 52.923 | 3.146 | 1.00 | 24.83 |
| ATOM | 3918 | CB | GLN | B | 699 | 30.510 | 53.143 | 1.633 | 1.00 | 34.19 |
| ATOM | 3919 | CG | GLN | B | 699 | 29.191 | 53.687 | 1.093 | 1.00 | 34.82 |
| ATOM | 3920 | CD | GLN | B | 699 | 29.205 | 53.911 | −0.399 | 1.00 | 35.20 |
| ATOM | 3921 | OE1 | GLN | B | 699 | 29.346 | 52.971 | −1.178 | 1.00 | 35.77 |
| ATOM | 3922 | NE2 | GLN | B | 699 | 29.056 | 55.163 | −0.808 | 1.00 | 35.33 |
| ATOM | 3923 | C | GLN | B | 699 | 31.819 | 52.493 | 3.683 | 1.00 | 23.99 |
| ATOM | 3924 | O | GLN | B | 699 | 32.860 | 52.768 | 3.083 | 1.00 | 25.36 |
| ATOM | 3925 | N | ILE | B | 700 | 31.791 | 51.794 | 4.814 | 1.00 | 20.88 |
| ATOM | 3926 | CA | ILE | B | 700 | 32.996 | 51.309 | 5.475 | 1.00 | 21.48 |
| ATOM | 3927 | CB | ILE | B | 700 | 32.696 | 50.857 | 6.919 | 1.00 | 24.53 |
| ATOM | 3928 | CG2 | ILE | B | 700 | 32.254 | 52.056 | 7.759 | 1.00 | 24.80 |
| ATOM | 3929 | CG1 | ILE | B | 700 | 31.623 | 49.765 | 6.917 | 1.00 | 24.49 |
| ATOM | 3930 | CD1 | ILE | B | 700 | 31.415 | 49.110 | 8.268 | 1.00 | 24.76 |
| ATOM | 3931 | C | ILE | B | 700 | 33.674 | 50.154 | 4.740 | 1.00 | 20.59 |
| ATOM | 3932 | O | ILE | B | 700 | 34.754 | 49.717 | 5.141 | 1.00 | 22.27 |
| ATOM | 3933 | N | LEU | B | 701 | 33.051 | 49.665 | 3.669 | 1.00 | 23.58 |
| ATOM | 3934 | CA | LEU | B | 701 | 33.627 | 48.571 | 2.892 | 1.00 | 23.94 |
| ATOM | 3935 | CB | LEU | B | 701 | 32.609 | 47.433 | 2.711 | 1.00 | 22.71 |
| ATOM | 3936 | CG | LEU | B | 701 | 32.080 | 46.709 | 3.955 | 1.00 | 22.35 |
| ATOM | 3937 | CD1 | LEU | B | 701 | 31.078 | 45.635 | 3.527 | 1.00 | 22.17 |
| ATOM | 3938 | CD2 | LEU | B | 701 | 33.235 | 46.087 | 4.732 | 1.00 | 22.94 |
| ATOM | 3939 | C | LEU | B | 701 | 34.058 | 49.086 | 1.523 | 1.00 | 25.02 |
| ATOM | 3940 | O | LEU | B | 701 | 34.425 | 48.308 | 0.643 | 1.00 | 25.13 |
| ATOM | 3941 | N | SER | B | 702 | 34.020 | 50.404 | 1.354 | 1.00 | 28.81 |
| ATOM | 3942 | CA | SER | B | 702 | 34.383 | 51.035 | 0.087 | 1.00 | 30.23 |
| ATOM | 3943 | CB | SER | B | 702 | 34.177 | 52.549 | 0.174 | 1.00 | 36.19 |
| ATOM | 3944 | OG | SER | B | 702 | 35.139 | 53.137 | 1.032 | 1.00 | 36.39 |
| ATOM | 3945 | C | SER | B | 702 | 35.825 | 50.761 | −0.323 | 1.00 | 30.61 |
| ATOM | 3946 | O | SER | B | 702 | 36.154 | 50.788 | −1.511 | 1.00 | 30.09 |
| ATOM | 3947 | N | GLY | B | 703 | 36.680 | 50.504 | 0.661 | 1.00 | 30.88 |
| ATOM | 3948 | CA | GLY | B | 703 | 38.083 | 50.258 | 0.379 | 1.00 | 30.78 |
| ATOM | 3949 | C | GLY | B | 703 | 38.446 | 48.843 | −0.029 | 1.00 | 31.56 |
| ATOM | 3950 | O | GLY | B | 703 | 39.570 | 48.595 | −0.461 | 1.00 | 31.02 |
| ATOM | 3951 | N | LEU | B | 704 | 37.506 | 47.914 | 0.109 | 1.00 | 27.62 |
| ATOM | 3952 | CA | LEU | B | 704 | 37.748 | 46.522 | −0.252 | 1.00 | 28.14 |
| ATOM | 3953 | CB | LEU | B | 704 | 36.638 | 45.628 | 0.312 | 1.00 | 24.97 |
| ATOM | 3954 | CG | LEU | B | 704 | 36.734 | 45.205 | 1.775 | 1.00 | 25.06 |
| ATOM | 3955 | CD1 | LEU | B | 704 | 35.542 | 44.349 | 2.161 | 1.00 | 25.71 |
| ATOM | 3956 | CD2 | LEU | B | 704 | 38.002 | 44.413 | 1.965 | 1.00 | 26.03 |
| ATOM | 3957 | C | LEU | B | 704 | 37.834 | 46.306 | −1.760 | 1.00 | 27.32 |
| ATOM | 3958 | O | LEU | B | 704 | 37.211 | 47.026 | −2.537 | 1.00 | 27.75 |
| ATOM | 3959 | N | SER | B | 705 | 38.610 | 45.305 | −2.164 | 1.00 | 25.03 |
| ATOM | 3960 | CA | SER | B | 705 | 38.740 | 44.982 | −3.576 | 1.00 | 25.33 |
| ATOM | 3961 | CB | SER | B | 705 | 39.909 | 44.019 | −3.807 | 1.00 | 22.63 |
| ATOM | 3962 | OG | SER | B | 705 | 39.647 | 42.746 | −3.249 | 1.00 | 22.15 |
| ATOM | 3963 | C | SER | B | 705 | 37.428 | 44.308 | −3.952 | 1.00 | 25.55 |
| ATOM | 3964 | O | SER | B | 705 | 36.645 | 43.950 | −3.073 | 1.00 | 25.89 |
| ATOM | 3965 | N | ILE | B | 706 | 37.190 | 44.134 | −5.248 | 1.00 | 28.89 |
| ATOM | 3966 | CA | ILE | B | 706 | 35.960 | 43.503 | −5.712 | 1.00 | 30.40 |
| ATOM | 3967 | CB | ILE | B | 706 | 35.967 | 43.351 | −7.249 | 1.00 | 38.06 |
| ATOM | 3968 | CG2 | ILE | B | 706 | 34.756 | 42.548 | −7.701 | 1.00 | 38.45 |
| ATOM | 3969 | CG1 | ILE | B | 706 | 35.984 | 44.737 | −7.900 | 1.00 | 38.67 |
| ATOM | 3970 | CD1 | ILE | B | 706 | 36.141 | 44.717 | −9.410 | 1.00 | 38.81 |
| ATOM | 3971 | C | ILE | B | 706 | 35.735 | 42.134 | −5.070 | 1.00 | 29.62 |
| ATOM | 3972 | O | ILE | B | 706 | 34.697 | 41.897 | −4.452 | 1.00 | 29.75 |
| ATOM | 3973 | N | GLU | B | 707 | 36.704 | 41.234 | −5.210 | 1.00 | 26.84 |
| ATOM | 3974 | CA | GLU | B | 707 | 36.579 | 39.900 | −4.631 | 1.00 | 26.37 |
| ATOM | 3975 | CB | GLU | B | 707 | 37.811 | 39.056 | −4.958 | 1.00 | 46.28 |
| ATOM | 3976 | CG | GLU | B | 707 | 37.665 | 38.188 | −6.198 | 1.00 | 47.22 |
| ATOM | 3977 | CD | GLU | B | 707 | 36.441 | 37.287 | −6.135 | 1.00 | 47.50 |
| ATOM | 3978 | OE1 | GLU | B | 707 | 35.368 | 37.699 | −6.626 | 1.00 | 47.38 |
| ATOM | 3979 | OE2 | GLU | B | 707 | 36.548 | 36.171 | −5.581 | 1.00 | 47.48 |
| ATOM | 3980 | C | GLU | B | 707 | 36.372 | 39.927 | −3.121 | 1.00 | 25.62 |
| ATOM | 3981 | O | GLU | B | 707 | 35.505 | 39.227 | −2.591 | 1.00 | 24.84 |
| ATOM | 3982 | N | GLU | B | 708 | 37.175 | 40.723 | −2.419 | 1.00 | 24.00 |
| ATOM | 3983 | CA | GLU | B | 708 | 37.031 | 40.803 | −0.974 | 1.00 | 22.95 |
| ATOM | 3984 | CB | GLU | B | 708 | 38.126 | 41.683 | −0.365 | 1.00 | 34.88 |
| ATOM | 3985 | CG | GLU | B | 708 | 39.451 | 40.952 | −0.225 | 1.00 | 34.89 |
| ATOM | 3986 | CD | GLU | B | 708 | 40.418 | 41.646 | 0.714 | 1.00 | 35.05 |
| ATOM | 3987 | OE1 | GLU | B | 708 | 41.368 | 40.981 | 1.167 | 1.00 | 35.66 |
| ATOM | 3988 | OE2 | GLU | B | 708 | 40.235 | 42.846 | 0.994 | 1.00 | 35.78 |
| ATOM | 3989 | C | GLU | B | 708 | 35.654 | 41.327 | −0.579 | 1.00 | 23.54 |
| ATOM | 3990 | O | GLU | B | 708 | 35.049 | 40.824 | 0.368 | 1.00 | 23.08 |
| ATOM | 3991 | N | TYR | B | 709 | 35.169 | 42.332 | −1.302 | 1.00 | 22.88 |
| ATOM | 3992 | CA | TYR | B | 709 | 33.855 | 42.908 | −1.022 | 1.00 | 23.56 |
| ATOM | 3993 | CB | TYR | B | 709 | 33.583 | 44.072 | −1.975 | 1.00 | 28.79 |
| ATOM | 3994 | CG | TYR | B | 709 | 32.228 | 44.708 | −1.784 | 1.00 | 29.56 |
| ATOM | 3995 | CD1 | TYR | B | 709 | 31.950 | 45.476 | −0.656 | 1.00 | 29.43 |
| ATOM | 3996 | CE1 | TYR | B | 709 | 30.691 | 46.039 | −0.461 | 1.00 | 29.53 |
| ATOM | 3997 | CD2 | TYR | B | 709 | 31.214 | 44.516 | −2.718 | 1.00 | 30.32 |
| ATOM | 3998 | CE2 | TYR | B | 709 | 29.950 | 45.072 | −2.533 | 1.00 | 29.63 |
| ATOM | 3999 | CZ | TYR | B | 709 | 29.696 | 45.830 | −1.404 | 1.00 | 30.45 |
| ATOM | 4000 | OH | TYR | B | 709 | 28.447 | 46.375 | −1.221 | 1.00 | 30.77 |
| ATOM | 4001 | C | TYR | B | 709 | 32.764 | 41.841 | −1.174 | 1.00 | 24.17 |
| ATOM | 4002 | O | TYR | B | 709 | 31.909 | 41.677 | −0.297 | 1.00 | 23.90 |
| ATOM | 4003 | N | LYS | B | 710 | 32.800 | 41.117 | −2.289 | 1.00 | 25.48 |
| ATOM | 4004 | CA | LYS | B | 710 | 31.821 | 40.062 | −2.543 | 1.00 | 27.03 |
| ATOM | 4005 | CB | LYS | B | 710 | 32.064 | 39.418 | −3.910 | 1.00 | 49.22 |
| ATOM | 4006 | CG | LYS | B | 710 | 31.519 | 37.999 | −3.992 | 1.00 | 50.09 |
| ATOM | 4007 | CD | LYS | B | 710 | 31.985 | 37.258 | −5.227 | 1.00 | 50.44 |
| ATOM | 4008 | CE | LYS | B | 710 | 31.769 | 35.762 | −5.057 | 1.00 | 50.21 |
| ATOM | 4009 | NZ | LYS | B | 710 | 30.379 | 35.439 | −4.621 | 1.00 | 50.37 |
| ATOM | 4010 | C | LYS | B | 710 | 31.872 | 38.972 | −1.480 | 1.00 | 25.65 |
| ATOM | 4011 | O | LYS | B | 710 | 30.840 | 38.500 | −0.998 | 1.00 | 26.50 |
| ATOM | 4012 | N | THR | B | 711 | 33.081 | 38.556 | −1.128 | 1.00 | 23.83 |
| ATOM | 4013 | CA | THR | B | 711 | 33.249 | 37.511 | −0.135 | 1.00 | 22.67 |
| ATOM | 4014 | CB | THR | B | 711 | 34.727 | 37.120 | −0.008 | 1.00 | 25.01 |
| ATOM | 4015 | OG1 | THR | B | 711 | 35.193 | 36.642 | −1.275 | 1.00 | 25.50 |
| ATOM | 4016 | CG2 | THR | B | 711 | 34.904 | 36.029 | 1.038 | 1.00 | 24.57 |
| ATOM | 4017 | C | THR | B | 711 | 32.723 | 37.944 | 1.232 | 1.00 | 22.92 |
| ATOM | 4018 | O | THR | B | 711 | 32.095 | 37.157 | 1.940 | 1.00 | 23.60 |
| ATOM | 4019 | N | THR | B | 712 | 32.975 | 39.202 | 1.586 | 1.00 | 20.46 |
| ATOM | 4020 | CA | THR | B | 712 | 32.551 | 39.742 | 2.871 | 1.00 | 20.53 |
| ATOM | 4021 | CB | THR | B | 712 | 33.201 | 41.117 | 3.125 | 1.00 | 22.65 |
| ATOM | 4022 | OG1 | THR | B | 712 | 34.624 | 40.993 | 3.023 | 1.00 | 23.36 |
| ATOM | 4023 | CG2 | THR | B | 712 | 32.849 | 41.628 | 4.515 | 1.00 | 22.01 |
| ATOM | 4024 | C | THR | B | 712 | 31.030 | 39.887 | 2.964 | 1.00 | 20.03 |
| ATOM | 4025 | O | THR | B | 712 | 30.433 | 39.564 | 3.992 | 1.00 | 19.56 |
| ATOM | 4026 | N | LEU | B | 713 | 30.405 | 40.375 | 1.899 | 1.00 | 20.53 |
| ATOM | 4027 | CA | LEU | B | 713 | 28.957 | 40.551 | 1.907 | 1.00 | 21.14 |
| ATOM | 4028 | CB | LEU | B | 713 | 28.484 | 41.186 | 0.599 | 1.00 | 32.94 |
| ATOM | 4029 | CG | LEU | B | 713 | 27.729 | 42.511 | 0.729 | 1.00 | 34.20 |
| ATOM | 4030 | CD1 | LEU | B | 713 | 26.628 | 42.370 | 1.774 | 1.00 | 34.24 |
| ATOM | 4031 | CD2 | LEU | B | 713 | 28.684 | 43.616 | 1.134 | 1.00 | 34.00 |
| ATOM | 4032 | C | LEU | B | 713 | 28.263 | 39.212 | 2.116 | 1.00 | 21.40 |
| ATOM | 4033 | O | LEU | B | 713 | 27.265 | 39.117 | 2.834 | 1.00 | 19.21 |
| ATOM | 4034 | N | LYS | B | 714 | 28.795 | 38.171 | 1.488 | 1.00 | 20.01 |
| ATOM | 4035 | CA | LYS | B | 714 | 28.220 | 36.837 | 1.620 | 1.00 | 20.38 |
| ATOM | 4036 | CB | LYS | B | 714 | 28.920 | 35.876 | 0.657 | 1.00 | 40.77 |
| ATOM | 4037 | CG | LYS | B | 714 | 28.564 | 34.419 | 0.847 | 1.00 | 41.45 |
| ATOM | 4038 | CD | LYS | B | 714 | 29.316 | 33.548 | −0.149 | 1.00 | 42.11 |
| ATOM | 4039 | CE | LYS | B | 714 | 29.138 | 32.073 | 0.165 | 1.00 | 42.05 |
| ATOM | 4040 | NZ | LYS | B | 714 | 27.703 | 31.694 | 0.221 | 1.00 | 42.14 |
| ATOM | 4041 | C | LYS | B | 714 | 28.343 | 36.322 | 3.052 | 1.00 | 19.95 |
| ATOM | 4042 | O | LYS | B | 714 | 27.417 | 35.721 | 3.597 | 1.00 | 20.70 |
| ATOM | 4043 | N | ILE | B | 715 | 29.499 | 36.544 | 3.662 | 1.00 | 19.98 |
| ATOM | 4044 | CA | ILE | B | 715 | 29.705 | 36.101 | 5.029 | 1.00 | 18.62 |
| ATOM | 4045 | CB | ILE | B | 715 | 31.145 | 36.386 | 5.500 | 1.00 | 22.51 |
| ATOM | 4046 | CG2 | ILE | B | 715 | 31.317 | 35.943 | 6.939 | 1.00 | 21.50 |
| ATOM | 4047 | CG1 | ILE | B | 715 | 32.141 | 35.638 | 4.612 | 1.00 | 22.82 |
| ATOM | 4048 | CD1 | ILE | B | 715 | 33.571 | 36.088 | 4.812 | 1.00 | 22.76 |
| ATOM | 4049 | C | ILE | B | 715 | 28.732 | 36.838 | 5.955 | 1.00 | 18.65 |
| ATOM | 4050 | O | ILE | B | 715 | 28.190 | 36.245 | 6.882 | 1.00 | 17.75 |
| ATOM | 4051 | N | ILE | B | 716 | 28.528 | 38.127 | 5.693 | 1.00 | 18.42 |
| ATOM | 4052 | CA | ILE | B | 716 | 27.624 | 38.951 | 6.500 | 1.00 | 17.16 |
| ATOM | 4053 | CB | ILE | B | 716 | 27.676 | 40.424 | 6.040 | 1.00 | 19.95 |
| ATOM | 4054 | CG2 | ILE | B | 716 | 26.549 | 41.225 | 6.683 | 1.00 | 19.27 |
| ATOM | 4055 | CG1 | ILE | B | 716 | 29.037 | 41.024 | 6.404 | 1.00 | 18.80 |
| ATOM | 4056 | CD1 | ILE | B | 716 | 29.258 | 42.434 | 5.887 | 1.00 | 18.71 |
| ATOM | 4057 | C | ILE | B | 716 | 26.192 | 38.426 | 6.411 | 1.00 | 19.67 |
| ATOM | 4058 | O | ILE | B | 716 | 25.544 | 38.183 | 7.436 | 1.00 | 16.84 |
| ATOM | 4059 | N | LYS | B | 717 | 25.706 | 38.224 | 5.192 | 1.00 | 17.44 |
| ATOM | 4060 | CA | LYS | B | 717 | 24.344 | 37.721 | 5.040 | 1.00 | 19.23 |
| ATOM | 4061 | CB | LYS | B | 717 | 23.980 | 37.489 | 3.573 | 1.00 | 19.08 |
| ATOM | 4062 | CG | LYS | B | 717 | 22.575 | 36.915 | 3.425 | 1.00 | 19.36 |
| ATOM | 4063 | CD | LYS | B | 717 | 22.145 | 36.779 | 1.975 | 1.00 | 20.95 |
| ATOM | 4064 | CE | LYS | B | 717 | 20.713 | 36.253 | 1.906 | 1.00 | 20.68 |
| ATOM | 4065 | NZ | LYS | B | 717 | 20.177 | 36.245 | 0.520 | 1.00 | 22.57 |
| ATOM | 4066 | C | LYS | B | 717 | 24.167 | 36.408 | 5.797 | 1.00 | 18.60 |
| ATOM | 4067 | O | LYS | B | 717 | 23.204 | 36.240 | 6.539 | 1.00 | 19.65 |
| ATOM | 4068 | N | GLN | B | 718 | 25.091 | 35.469 | 5.602 | 1.00 | 18.67 |
| ATOM | 4069 | CA | GLN | B | 718 | 24.992 | 34.181 | 6.280 | 1.00 | 18.42 |
| ATOM | 4070 | CB | GLN | B | 718 | 26.110 | 33.256 | 5.794 | 1.00 | 35.34 |
| ATOM | 4071 | CG | GLN | B | 718 | 25.885 | 32.724 | 4.386 | 1.00 | 36.06 |
| ATOM | 4072 | CD | GLN | B | 718 | 27.121 | 32.072 | 3.798 | 1.00 | 36.81 |
| ATOM | 4073 | OE1 | GLN | B | 718 | 27.036 | 31.295 | 2.843 | 1.00 | 37.02 |
| ATOM | 4074 | NE2 | GLN | B | 718 | 28.282 | 32.397 | 4.356 | 1.00 | 36.65 |
| ATOM | 4075 | C | GLN | B | 718 | 25.039 | 34.298 | 7.802 | 1.00 | 16.64 |
| ATOM | 4076 | O | GLN | B | 718 | 24.337 | 33.577 | 8.517 | 1.00 | 17.20 |
| ATOM | 4077 | N | ALA | B | 719 | 25.855 | 35.221 | 8.299 | 1.00 | 17.42 |
| ATOM | 4078 | CA | ALA | B | 719 | 25.989 | 35.400 | 9.737 | 1.00 | 16.91 |
| ATOM | 4079 | CB | ALA | B | 719 | 27.181 | 36.314 | 10.037 | 1.00 | 18.95 |
| ATOM | 4080 | C | ALA | B | 719 | 24.705 | 35.979 | 10.338 | 1.00 | 16.46 |
| ATOM | 4081 | O | ALA | B | 719 | 24.280 | 35.566 | 11.415 | 1.00 | 16.41 |
| ATOM | 4082 | N | ILE | B | 720 | 24.087 | 36.923 | 9.632 | 1.00 | 16.16 |
| ATOM | 4083 | CA | ILE | B | 720 | 22.848 | 37.517 | 10.128 | 1.00 | 18.05 |
| ATOM | 4084 | CB | ILE | B | 720 | 22.463 | 38.775 | 9.317 | 1.00 | 16.77 |
| ATOM | 4085 | CG2 | ILE | B | 720 | 21.021 | 39.200 | 9.634 | 1.00 | 17.00 |
| ATOM | 4086 | CG1 | ILE | B | 720 | 23.458 | 39.903 | 9.626 | 1.00 | 17.24 |
| ATOM | 4087 | CD1 | ILE | B | 720 | 23.165 | 41.216 | 8.938 | 1.00 | 18.75 |
| ATOM | 4088 | C | ILE | B | 720 | 21.725 | 36.484 | 10.083 | 1.00 | 16.85 |
| ATOM | 4089 | O | ILE | B | 720 | 20.966 | 36.339 | 11.044 | 1.00 | 17.13 |
| ATOM | 4090 | N | LEU | B | 721 | 21.628 | 35.741 | 8.983 | 1.00 | 17.26 |
| ATOM | 4091 | CA | LEU | B | 721 | 20.588 | 34.726 | 8.886 | 1.00 | 17.99 |
| ATOM | 4092 | CB | LEU | B | 721 | 20.611 | 34.056 | 7.507 | 1.00 | 21.38 |
| ATOM | 4093 | CG | LEU | B | 721 | 19.998 | 34.910 | 6.390 | 1.00 | 22.95 |
| ATOM | 4094 | CD1 | LEU | B | 721 | 20.102 | 34.181 | 5.071 | 1.00 | 22.96 |
| ATOM | 4095 | CD2 | LEU | B | 721 | 18.544 | 35.204 | 6.707 | 1.00 | 22.88 |
| ATOM | 4096 | C | LEU | B | 721 | 20.764 | 33.686 | 9.984 | 1.00 | 17.50 |
| ATOM | 4097 | O | LEU | B | 721 | 19.791 | 33.142 | 10.493 | 1.00 | 18.24 |
| ATOM | 4098 | N | ALA | B | 722 | 22.013 | 33.425 | 10.359 | 1.00 | 18.93 |
| ATOM | 4099 | CA | ALA | B | 722 | 22.305 | 32.448 | 11.398 | 1.00 | 19.70 |
| ATOM | 4100 | CB | ALA | B | 722 | 23.818 | 32.282 | 11.556 | 1.00 | 22.76 |
| ATOM | 4101 | C | ALA | B | 722 | 21.689 | 32.803 | 12.748 | 1.00 | 19.06 |
| ATOM | 4102 | O | ALA | B | 722 | 21.491 | 31.926 | 13.581 | 1.00 | 19.32 |
| ATOM | 4103 | N | THR | B | 723 | 21.389 | 34.081 | 12.972 | 1.00 | 19.12 |
| ATOM | 4104 | CA | THR | B | 723 | 20.820 | 34.484 | 14.255 | 1.00 | 18.93 |
| ATOM | 4105 | CB | THR | B | 723 | 20.997 | 36.000 | 14.503 | 1.00 | 16.46 |
| ATOM | 4106 | OG1 | THR | B | 723 | 20.359 | 36.746 | 13.463 | 1.00 | 18.08 |
| ATOM | 4107 | CG2 | THR | B | 723 | 22.485 | 36.355 | 14.536 | 1.00 | 17.70 |
| ATOM | 4108 | C | THR | B | 723 | 19.352 | 34.093 | 14.440 | 1.00 | 20.30 |
| ATOM | 4109 | O | THR | B | 723 | 18.744 | 34.373 | 15.474 | 1.00 | 18.88 |
| ATOM | 4110 | N | ASP | B | 724 | 18.785 | 33.464 | 13.421 | 1.00 | 20.75 |
| ATOM | 4111 | CA | ASP | B | 724 | 17.417 | 32.977 | 13.495 | 1.00 | 21.45 |
| ATOM | 4112 | CB | ASP | B | 724 | 16.901 | 32.670 | 12.084 | 1.00 | 20.47 |
| ATOM | 4113 | CG | ASP | B | 724 | 15.541 | 31.991 | 12.091 | 1.00 | 19.47 |
| ATOM | 4114 | OD1 | ASP | B | 724 | 14.982 | 31.769 | 13.183 | 1.00 | 20.37 |
| ATOM | 4115 | OD2 | ASP | B | 724 | 15.029 | 31.683 | 10.995 | 1.00 | 20.97 |
| ATOM | 4116 | C | ASP | B | 724 | 17.571 | 31.684 | 14.296 | 1.00 | 22.80 |
| ATOM | 4117 | O | ASP | B | 724 | 18.171 | 30.729 | 13.803 | 1.00 | 21.11 |
| ATOM | 4118 | N | LEU | B | 725 | 17.057 | 31.648 | 15.524 | 1.00 | 21.88 |
| ATOM | 4119 | CA | LEU | B | 725 | 17.211 | 30.454 | 16.354 | 1.00 | 23.26 |
| ATOM | 4120 | CB | LEU | B | 725 | 16.575 | 30.659 | 17.734 | 1.00 | 21.15 |
| ATOM | 4121 | CG | LEU | B | 725 | 17.439 | 31.482 | 18.698 | 1.00 | 22.29 |
| ATOM | 4122 | CD1 | LEU | B | 725 | 16.712 | 31.674 | 20.019 | 1.00 | 22.69 |
| ATOM | 4123 | CD2 | LEU | B | 725 | 18.768 | 30.765 | 18.927 | 1.00 | 22.08 |
| ATOM | 4124 | C | LEU | B | 725 | 16.685 | 29.178 | 15.720 | 1.00 | 24.43 |
| ATOM | 4125 | O | LEU | B | 725 | 17.084 | 28.074 | 16.110 | 1.00 | 24.43 |
| ATOM | 4126 | N | ALA | B | 726 | 15.802 | 29.322 | 14.735 | 1.00 | 24.46 |
| ATOM | 4127 | CA | ALA | B | 726 | 15.256 | 28.158 | 14.042 | 1.00 | 24.85 |
| ATOM | 4128 | CB | ALA | B | 726 | 14.096 | 28.576 | 13.144 | 1.00 | 26.10 |
| ATOM | 4129 | C | ALA | B | 726 | 16.361 | 27.502 | 13.219 | 1.00 | 25.58 |
| ATOM | 4130 | O | ALA | B | 726 | 16.436 | 26.277 | 13.127 | 1.00 | 24.81 |
| ATOM | 4131 | N | LEU | B | 727 | 17.217 | 28.323 | 12.617 | 1.00 | 25.66 |
| ATOM | 4132 | CA | LEU | B | 727 | 18.332 | 27.826 | 11.825 | 1.00 | 26.45 |
| ATOM | 4133 | CB | LEU | B | 727 | 18.952 | 28.969 | 11.016 | 1.00 | 32.52 |
| ATOM | 4134 | CG | LEU | B | 727 | 18.730 | 28.953 | 9.499 | 1.00 | 34.10 |
| ATOM | 4135 | CD1 | LEU | B | 727 | 17.320 | 28.490 | 9.163 | 1.00 | 32.76 |
| ATOM | 4136 | CD2 | LEU | B | 727 | 18.999 | 30.343 | 8.938 | 1.00 | 32.91 |
| ATOM | 4137 | C | LEU | B | 727 | 19.370 | 27.216 | 12.767 | 1.00 | 26.66 |
| ATOM | 4138 | O | LEU | B | 727 | 19.994 | 26.208 | 12.448 | 1.00 | 27.25 |
| ATOM | 4139 | N | TYR | B | 728 | 19.549 | 27.828 | 13.933 | 1.00 | 23.88 |
| ATOM | 4140 | CA | TYR | B | 728 | 20.497 | 27.311 | 14.912 | 1.00 | 24.96 |
| ATOM | 4141 | CB | TYR | B | 728 | 20.576 | 28.246 | 16.127 | 1.00 | 22.32 |
| ATOM | 4142 | CG | TYR | B | 728 | 21.197 | 27.606 | 17.348 | 1.00 | 22.57 |
| ATOM | 4143 | CD1 | TYR | B | 728 | 20.402 | 27.173 | 18.410 | 1.00 | 23.27 |
| ATOM | 4144 | CE1 | TYR | B | 728 | 20.958 | 26.520 | 19.512 | 1.00 | 22.36 |
| ATOM | 4145 | CD2 | TYR | B | 728 | 22.569 | 27.372 | 17.416 | 1.00 | 23.33 |
| ATOM | 4146 | CE2 | TYR | B | 728 | 23.133 | 26.713 | 18.512 | 1.00 | 22.71 |
| ATOM | 4147 | CZ | TYR | B | 728 | 22.322 | 26.292 | 19.553 | 1.00 | 23.34 |
| ATOM | 4148 | OH | TYR | B | 728 | 22.876 | 25.642 | 20.638 | 1.00 | 25.15 |
| ATOM | 4149 | C | TYR | B | 728 | 20.085 | 25.908 | 15.365 | 1.00 | 25.55 |
| ATOM | 4150 | O | TYR | B | 728 | 20.894 | 24.982 | 15.368 | 1.00 | 25.79 |
| ATOM | 4151 | N | ILE | B | 729 | 18.821 | 25.759 | 15.747 | 1.00 | 27.10 |
| ATOM | 4152 | CA | ILE | B | 729 | 18.313 | 24.476 | 16.218 | 1.00 | 27.90 |
| ATOM | 4153 | CB | ILE | B | 729 | 16.849 | 24.608 | 16.692 | 1.00 | 33.37 |
| ATOM | 4154 | CG2 | ILE | B | 729 | 16.245 | 23.231 | 16.936 | 1.00 | 34.11 |
| ATOM | 4155 | CG1 | ILE | B | 729 | 16.800 | 25.447 | 17.969 | 1.00 | 33.59 |
| ATOM | 4156 | CD1 | ILE | B | 729 | 15.400 | 25.662 | 18.512 | 1.00 | 34.44 |
| ATOM | 4157 | C | ILE | B | 729 | 18.396 | 23.395 | 15.151 | 1.00 | 28.43 |
| ATOM | 4158 | O | ILE | B | 729 | 18.561 | 22.212 | 15.463 | 1.00 | 28.49 |
| ATOM | 4159 | N | LYS | B | 730 | 18.286 | 23.803 | 13.893 | 1.00 | 30.10 |
| ATOM | 4160 | CA | LYS | B | 730 | 18.335 | 22.865 | 12.777 | 1.00 | 30.87 |
| ATOM | 4161 | CB | LYS | B | 730 | 17.637 | 23.474 | 11.554 | 1.00 | 41.47 |
| ATOM | 4162 | CG | LYS | B | 730 | 17.736 | 22.627 | 10.292 | 1.00 | 41.83 |
| ATOM | 4163 | CD | LYS | B | 730 | 17.082 | 23.312 | 9.099 | 1.00 | 41.85 |
| ATOM | 4164 | CE | LYS | B | 730 | 17.161 | 22.437 | 7.856 | 1.00 | 42.29 |
| ATOM | 4165 | NZ | LYS | B | 730 | 16.471 | 23.056 | 6.689 | 1.00 | 41.92 |
| ATOM | 4166 | C | LYS | B | 730 | 19.753 | 22.450 | 12.390 | 1.00 | 30.30 |
| ATOM | 4167 | O | LYS | B | 730 | 19.968 | 21.341 | 11.899 | 1.00 | 30.78 |
| ATOM | 4168 | N | ARG | B | 731 | 20.722 | 23.330 | 12.611 | 1.00 | 26.44 |
| ATOM | 4169 | CA | ARG | B | 731 | 22.101 | 23.029 | 12.234 | 1.00 | 26.05 |
| ATOM | 4170 | CB | ARG | B | 731 | 22.691 | 24.207 | 11.455 | 1.00 | 44.87 |
| ATOM | 4171 | CG | ARG | B | 731 | 22.027 | 24.477 | 10.123 | 1.00 | 45.35 |
| ATOM | 4172 | CD | ARG | B | 731 | 22.579 | 25.739 | 9.481 | 1.00 | 45.70 |
| ATOM | 4173 | NE | ARG | B | 731 | 24.018 | 25.666 | 9.236 | 1.00 | 45.72 |
| ATOM | 4174 | CZ | ARG | B | 731 | 24.600 | 24.771 | 8.445 | 1.00 | 45.64 |
| ATOM | 4175 | NH1 | ARG | B | 731 | 23.870 | 23.859 | 7.816 | 1.00 | 45.97 |
| ATOM | 4176 | NH2 | ARG | B | 731 | 25.915 | 24.793 | 8.272 | 1.00 | 45.83 |
| ATOM | 4177 | C | ARG | B | 731 | 23.076 | 22.657 | 13.349 | 1.00 | 25.87 |
| ATOM | 4178 | O | ARG | B | 731 | 24.144 | 22.121 | 13.069 | 1.00 | 25.60 |
| ATOM | 4179 | N | ARG | B | 732 | 22.724 | 22.931 | 14.600 | 1.00 | 24.75 |
| ATOM | 4180 | CA | ARG | B | 732 | 23.636 | 22.646 | 15.704 | 1.00 | 24.59 |
| ATOM | 4181 | CB | ARG | B | 732 | 23.051 | 23.154 | 17.021 | 1.00 | 28.71 |
| ATOM | 4182 | CG | ARG | B | 732 | 21.825 | 22.412 | 17.490 | 1.00 | 28.36 |
| ATOM | 4183 | CD | ARG | B | 732 | 21.266 | 23.052 | 18.742 | 1.00 | 28.99 |
| ATOM | 4184 | NE | ARG | B | 732 | 20.160 | 22.282 | 19.295 | 1.00 | 28.09 |
| ATOM | 4185 | CZ | ARG | B | 732 | 19.555 | 22.567 | 20.444 | 1.00 | 29.06 |
| ATOM | 4186 | NH1 | ARG | B | 732 | 19.951 | 23.607 | 21.164 | 1.00 | 29.87 |
| ATOM | 4187 | NH2 | ARG | B | 732 | 18.560 | 21.807 | 20.878 | 1.00 | 29.81 |
| ATOM | 4188 | C | ARG | B | 732 | 24.027 | 21.177 | 15.845 | 1.00 | 24.59 |
| ATOM | 4189 | O | ARG | B | 732 | 25.143 | 20.871 | 16.272 | 1.00 | 24.56 |
| ATOM | 4190 | N | GLY | B | 733 | 23.114 | 20.278 | 15.495 | 1.00 | 28.28 |
| ATOM | 4191 | CA | GLY | B | 733 | 23.409 | 18.857 | 15.592 | 1.00 | 29.55 |
| ATOM | 4192 | C | GLY | B | 733 | 24.686 | 18.506 | 14.852 | 1.00 | 29.73 |
| ATOM | 4193 | O | GLY | B | 733 | 25.582 | 17.856 | 15.402 | 1.00 | 29.54 |
| ATOM | 4194 | N | GLU | B | 734 | 24.775 | 18.940 | 13.599 | 1.00 | 29.63 |
| ATOM | 4195 | CA | GLU | B | 734 | 25.954 | 18.676 | 12.779 | 1.00 | 29.74 |
| ATOM | 4196 | CB | GLU | B | 734 | 25.772 | 19.317 | 11.396 | 1.00 | 37.09 |
| ATOM | 4197 | CG | GLU | B | 734 | 27.011 | 19.313 | 10.513 | 1.00 | 37.69 |
| ATOM | 4198 | CD | GLU | B | 734 | 26.712 | 19.749 | 9.090 | 1.00 | 37.69 |
| ATOM | 4199 | OE1 | GLU | B | 734 | 25.895 | 20.673 | 8.905 | 1.00 | 37.77 |
| ATOM | 4200 | OE2 | GLU | B | 734 | 27.302 | 19.174 | 8.153 | 1.00 | 37.96 |
| ATOM | 4201 | C | GLU | B | 734 | 27.234 | 19.187 | 13.448 | 1.00 | 29.89 |
| ATOM | 4202 | O | GLU | B | 734 | 28.241 | 18.481 | 13.508 | 1.00 | 29.99 |
| ATOM | 4203 | N | PHE | B | 735 | 27.193 | 20.416 | 13.953 | 1.00 | 26.19 |
| ATOM | 4204 | CA | PHE | B | 735 | 28.348 | 21.015 | 14.617 | 1.00 | 28.01 |
| ATOM | 4205 | CB | PHE | B | 735 | 28.001 | 22.449 | 15.051 | 1.00 | 32.46 |
| ATOM | 4206 | CG | PHE | B | 735 | 29.121 | 23.170 | 15.748 | 1.00 | 32.58 |
| ATOM | 4207 | CD1 | PHE | B | 735 | 30.350 | 23.353 | 15.121 | 1.00 | 32.98 |
| ATOM | 4208 | CD2 | PHE | B | 735 | 28.930 | 23.710 | 17.018 | 1.00 | 32.86 |
| ATOM | 4209 | CE1 | PHE | B | 735 | 31.374 | 24.067 | 15.746 | 1.00 | 33.43 |
| ATOM | 4210 | CE2 | PHE | B | 735 | 29.941 | 24.424 | 17.653 | 1.00 | 33.17 |
| ATOM | 4211 | CZ | PHE | B | 735 | 31.168 | 24.606 | 17.017 | 1.00 | 33.32 |
| ATOM | 4212 | C | PHE | B | 735 | 28.759 | 20.185 | 15.832 | 1.00 | 28.38 |
| ATOM | 4213 | O | PHE | B | 735 | 29.943 | 19.877 | 16.023 | 1.00 | 28.13 |
| ATOM | 4214 | N | PHE | B | 736 | 27.772 | 19.823 | 16.646 | 1.00 | 35.56 |
| ATOM | 4215 | CA | PHE | B | 736 | 28.008 | 19.033 | 17.848 | 1.00 | 36.50 |
| ATOM | 4216 | CB | PHE | B | 736 | 26.695 | 18.843 | 18.615 | 1.00 | 29.57 |
| ATOM | 4217 | CG | PHE | B | 736 | 26.153 | 20.118 | 19.217 | 1.00 | 28.48 |
| ATOM | 4218 | CD1 | PHE | B | 736 | 24.849 | 20.174 | 19.707 | 1.00 | 29.25 |
| ATOM | 4219 | CD2 | PHE | B | 736 | 26.947 | 21.261 | 19.298 | 1.00 | 28.68 |
| ATOM | 4220 | CE1 | PHE | B | 736 | 24.343 | 21.351 | 20.270 | 1.00 | 28.48 |
| ATOM | 4221 | CE2 | PHE | B | 736 | 26.451 | 22.440 | 19.857 | 1.00 | 28.64 |
| ATOM | 4222 | CZ | PHE | B | 736 | 25.142 | 22.481 | 20.344 | 1.00 | 28.27 |
| ATOM | 4223 | C | PHE | B | 736 | 28.636 | 17.678 | 17.538 | 1.00 | 36.98 |
| ATOM | 4224 | O | PHE | B | 736 | 29.472 | 17.192 | 18.299 | 1.00 | 37.51 |
| ATOM | 4225 | N | GLU | B | 737 | 28.236 | 17.074 | 16.422 | 1.00 | 35.33 |
| ATOM | 4226 | CA | GLU | B | 737 | 28.779 | 15.778 | 16.019 | 1.00 | 34.66 |
| ATOM | 4227 | CB | GLU | B | 737 | 28.042 | 15.245 | 14.788 | 1.00 | 48.23 |
| ATOM | 4228 | CG | GLU | B | 737 | 26.624 | 14.782 | 15.052 | 1.00 | 48.59 |
| ATOM | 4229 | CD | GLU | B | 737 | 26.562 | 13.677 | 16.084 | 1.00 | 48.98 |
| ATOM | 4230 | OE1 | GLU | B | 737 | 27.347 | 12.710 | 15.969 | 1.00 | 48.80 |
| ATOM | 4231 | OE2 | GLU | B | 737 | 25.724 | 13.771 | 17.004 | 1.00 | 49.04 |
| ATOM | 4232 | C | GLU | B | 737 | 30.264 | 15.895 | 15.698 | 1.00 | 35.25 |
| ATOM | 4233 | O | GLU | B | 737 | 31.095 | 15.184 | 16.276 | 1.00 | 34.50 |
| ATOM | 4234 | N | LEU | B | 738 | 30.582 | 16.792 | 14.766 | 1.00 | 34.53 |
| ATOM | 4235 | CA | LEU | B | 738 | 31.958 | 17.036 | 14.339 | 1.00 | 34.99 |
| ATOM | 4236 | CB | LEU | B | 738 | 32.010 | 18.266 | 13.422 | 1.00 | 33.43 |
| ATOM | 4237 | CG | LEU | B | 738 | 31.532 | 18.137 | 11.969 | 1.00 | 33.44 |
| ATOM | 4238 | CD1 | LEU | B | 738 | 30.237 | 17.345 | 11.885 | 1.00 | 33.77 |
| ATOM | 4239 | CD2 | LEU | B | 738 | 31.345 | 19.531 | 11.388 | 1.00 | 33.47 |
| ATOM | 4240 | C | LEU | B | 738 | 32.889 | 17.243 | 15.530 | 1.00 | 35.52 |
| ATOM | 4241 | O | LEU | B | 738 | 34.020 | 16.760 | 15.535 | 1.00 | 35.19 |
| ATOM | 4242 | N | ILE | B | 739 | 32.403 | 17.960 | 16.538 | 1.00 | 35.96 |
| ATOM | 4243 | CA | ILE | B | 739 | 33.177 | 18.243 | 17.740 | 1.00 | 36.59 |
| ATOM | 4244 | CB | ILE | B | 739 | 32.489 | 19.347 | 18.578 | 1.00 | 41.13 |
| ATOM | 4245 | CG2 | ILE | B | 739 | 33.192 | 19.509 | 19.913 | 1.00 | 41.32 |
| ATOM | 4246 | CG1 | ILE | B | 739 | 32.478 | 20.665 | 17.798 | 1.00 | 41.45 |
| ATOM | 4247 | CD1 | ILE | B | 739 | 33.852 | 21.224 | 17.510 | 1.00 | 41.21 |
| ATOM | 4248 | C | ILE | B | 739 | 33.353 | 16.998 | 18.610 | 1.00 | 37.53 |
| ATOM | 4249 | O | ILE | B | 739 | 34.455 | 16.711 | 19.088 | 1.00 | 36.85 |
| ATOM | 4250 | N | ARG | B | 740 | 32.262 | 16.264 | 18.808 | 1.00 | 40.80 |
| ATOM | 4251 | CA | ARG | B | 740 | 32.276 | 15.058 | 19.627 | 1.00 | 41.57 |
| ATOM | 4252 | CB | ARG | B | 740 | 30.860 | 14.490 | 19.744 | 1.00 | 49.23 |
| ATOM | 4253 | CG | ARG | B | 740 | 30.708 | 13.372 | 20.763 | 1.00 | 49.50 |
| ATOM | 4254 | CD | ARG | B | 740 | 29.268 | 12.894 | 20.816 | 1.00 | 49.76 |
| ATOM | 4255 | NE | ARG | B | 740 | 28.870 | 12.228 | 19.577 | 1.00 | 49.63 |
| ATOM | 4256 | CZ | ARG | B | 740 | 29.192 | 10.977 | 19.264 | 1.00 | 49.97 |
| ATOM | 4257 | NH1 | ARG | B | 740 | 29.917 | 10.246 | 20.103 | 1.00 | 49.93 |
| ATOM | 4258 | NH2 | ARG | B | 740 | 28.795 | 10.456 | 18.111 | 1.00 | 49.81 |
| ATOM | 4259 | C | ARG | B | 740 | 33.202 | 13.999 | 19.045 | 1.00 | 41.31 |
| ATOM | 4260 | O | ARG | B | 740 | 34.041 | 13.437 | 19.750 | 1.00 | 41.75 |
| ATOM | 4261 | N | LYS | B | 741 | 33.048 | 13.734 | 17.753 | 1.00 | 36.01 |
| ATOM | 4262 | CA | LYS | B | 741 | 33.858 | 12.734 | 17.074 | 1.00 | 36.04 |
| ATOM | 4263 | CB | LYS | B | 741 | 33.142 | 12.261 | 15.806 | 1.00 | 51.86 |
| ATOM | 4264 | CG | LYS | B | 741 | 31.823 | 11.547 | 16.084 | 1.00 | 52.33 |
| ATOM | 4265 | CD | LYS | B | 741 | 31.198 | 10.984 | 14.819 | 1.00 | 52.60 |
| ATOM | 4266 | CE | LYS | B | 741 | 29.956 | 10.164 | 15.147 | 1.00 | 52.72 |
| ATOM | 4267 | NZ | LYS | B | 741 | 29.371 | 9.514 | 13.941 | 1.00 | 52.84 |
| ATOM | 4268 | C | LYS | B | 741 | 35.257 | 13.229 | 16.739 | 1.00 | 36.83 |
| ATOM | 4269 | O | LYS | B | 741 | 36.017 | 12.544 | 16.056 | 1.00 | 35.76 |
| ATOM | 4270 | N | ASN | B | 742 | 35.598 | 14.424 | 17.217 | 1.00 | 47.96 |
| ATOM | 4271 | CA | ASN | B | 742 | 36.925 | 14.991 | 16.979 | 1.00 | 48.89 |
| ATOM | 4272 | CB | ASN | B | 742 | 37.984 | 14.073 | 17.601 | 1.00 | 45.18 |
| ATOM | 4273 | CG | ASN | B | 742 | 37.660 | 13.696 | 19.038 | 1.00 | 45.25 |
| ATOM | 4274 | OD1 | ASN | B | 742 | 37.873 | 14.481 | 19.958 | 1.00 | 45.19 |
| ATOM | 4275 | ND2 | ASN | B | 742 | 37.127 | 12.492 | 19.231 | 1.00 | 45.61 |
| ATOM | 4276 | C | ASN | B | 742 | 37.210 | 15.160 | 15.482 | 1.00 | 48.30 |
| ATOM | 4277 | O | ASN | B | 742 | 38.318 | 14.886 | 15.023 | 1.00 | 49.56 |
| ATOM | 4278 | N | GLN | B | 743 | 36.214 | 15.627 | 14.733 | 1.00 | 35.63 |
| ATOM | 4279 | CA | GLN | B | 743 | 36.345 | 15.806 | 13.288 | 1.00 | 33.02 |
| ATOM | 4280 | CB | GLN | B | 743 | 35.226 | 15.034 | 12.583 | 1.00 | 55.29 |
| ATOM | 4281 | CG | GLN | B | 743 | 34.914 | 13.679 | 13.206 | 1.00 | 55.90 |
| ATOM | 4282 | CD | GLN | B | 743 | 33.531 | 13.159 | 12.830 | 1.00 | 56.30 |
| ATOM | 4283 | OE1 | GLN | B | 743 | 32.531 | 13.872 | 12.943 | 1.00 | 56.03 |
| ATOM | 4284 | NE2 | GLN | B | 743 | 33.470 | 11.903 | 12.396 | 1.00 | 56.30 |
| ATOM | 4285 | C | GLN | B | 743 | 36.296 | 17.272 | 12.833 | 1.00 | 32.63 |
| ATOM | 4286 | O | GLN | B | 743 | 36.519 | 17.568 | 11.659 | 1.00 | 30.98 |
| ATOM | 4287 | N | PHE | B | 744 | 36.008 | 18.186 | 13.754 | 1.00 | 30.07 |
| ATOM | 4288 | CA | PHE | B | 744 | 35.913 | 19.600 | 13.396 | 1.00 | 29.86 |
| ATOM | 4289 | CB | PHE | B | 744 | 35.664 | 20.451 | 14.646 | 1.00 | 36.04 |
| ATOM | 4290 | CG | PHE | B | 744 | 35.433 | 21.906 | 14.349 | 1.00 | 36.02 |
| ATOM | 4291 | CD1 | PHE | B | 744 | 34.426 | 22.294 | 13.472 | 1.00 | 36.51 |
| ATOM | 4292 | CD2 | PHE | B | 744 | 36.224 | 22.886 | 14.939 | 1.00 | 36.37 |
| ATOM | 4293 | CE1 | PHE | B | 744 | 34.210 | 23.641 | 13.186 | 1.00 | 36.33 |
| ATOM | 4294 | CE2 | PHE | B | 744 | 36.017 | 24.237 | 14.660 | 1.00 | 36.35 |
| ATOM | 4295 | CZ | PHE | B | 744 | 35.007 | 24.613 | 13.781 | 1.00 | 36.44 |
| ATOM | 4296 | C | PHE | B | 744 | 37.158 | 20.101 | 12.671 | 1.00 | 30.17 |
| ATOM | 4297 | O | PHE | B | 744 | 38.274 | 19.948 | 13.163 | 1.00 | 29.71 |
| ATOM | 4298 | N | ASN | B | 745 | 36.953 | 20.703 | 11.500 | 1.00 | 33.40 |
| ATOM | 4299 | CA | ASN | B | 745 | 38.047 | 21.229 | 10.685 | 1.00 | 34.97 |
| ATOM | 4300 | CB | ASN | B | 745 | 38.463 | 20.192 | 9.634 | 1.00 | 36.63 |
| ATOM | 4301 | CG | ASN | B | 745 | 39.397 | 20.765 | 8.586 | 1.00 | 36.73 |
| ATOM | 4302 | OD1 | ASN | B | 745 | 40.281 | 21.562 | 8.896 | 1.00 | 37.02 |
| ATOM | 4303 | ND2 | ASN | B | 745 | 39.216 | 20.345 | 7.337 | 1.00 | 37.05 |
| ATOM | 4304 | C | ASN | B | 745 | 37.674 | 22.537 | 9.992 | 1.00 | 34.71 |
| ATOM | 4305 | O | ASN | B | 745 | 36.930 | 22.540 | 9.011 | 1.00 | 34.81 |
| ATOM | 4306 | N | LEU | B | 746 | 38.209 | 23.645 | 10.495 | 1.00 | 32.32 |
| ATOM | 4307 | CA | LEU | B | 746 | 37.914 | 24.961 | 9.934 | 1.00 | 31.51 |
| ATOM | 4308 | CB | LEU | B | 746 | 38.459 | 26.059 | 10.853 | 1.00 | 35.95 |
| ATOM | 4309 | CG | LEU | B | 746 | 37.591 | 26.325 | 12.083 | 1.00 | 36.41 |
| ATOM | 4310 | CD1 | LEU | B | 746 | 38.239 | 27.390 | 12.951 | 1.00 | 36.26 |
| ATOM | 4311 | CD2 | LEU | B | 746 | 36.200 | 26.773 | 11.635 | 1.00 | 36.10 |
| ATOM | 4312 | C | LEU | B | 746 | 38.419 | 25.181 | 8.511 | 1.00 | 31.36 |
| ATOM | 4313 | O | LEU | B | 746 | 38.085 | 26.183 | 7.877 | 1.00 | 31.04 |
| ATOM | 4314 | N | GLU | B | 747 | 39.224 | 24.252 | 8.010 | 1.00 | 30.95 |
| ATOM | 4315 | CA | GLU | B | 747 | 39.738 | 24.357 | 6.650 | 1.00 | 31.86 |
| ATOM | 4316 | CB | GLU | B | 747 | 40.918 | 23.402 | 6.453 | 1.00 | 51.07 |
| ATOM | 4317 | CG | GLU | B | 747 | 42.129 | 23.738 | 7.311 | 1.00 | 51.73 |
| ATOM | 4318 | CD | GLU | B | 747 | 43.251 | 22.728 | 7.163 | 1.00 | 52.11 |
| ATOM | 4319 | OE1 | GLU | B | 747 | 43.666 | 22.467 | 6.014 | 1.00 | 52.03 |
| ATOM | 4320 | OE2 | GLU | B | 747 | 43.718 | 22.201 | 8.196 | 1.00 | 52.21 |
| ATOM | 4321 | C | GLU | B | 747 | 38.617 | 24.014 | 5.670 | 1.00 | 31.25 |
| ATOM | 4322 | O | GLU | B | 747 | 38.646 | 24.421 | 4.507 | 1.00 | 30.95 |
| ATOM | 4323 | N | ASP | B | 748 | 37.634 | 23.260 | 6.156 | 1.00 | 31.79 |
| ATOM | 4324 | CA | ASP | B | 748 | 36.480 | 22.864 | 5.357 | 1.00 | 31.93 |
| ATOM | 4325 | CB | ASP | B | 748 | 35.754 | 21.697 | 6.031 | 1.00 | 39.97 |
| ATOM | 4326 | CG | ASP | B | 748 | 34.571 | 21.193 | 5.223 | 1.00 | 40.51 |
| ATOM | 4327 | OD1 | ASP | B | 748 | 33.678 | 21.995 | 4.889 | 1.00 | 40.27 |
| ATOM | 4328 | OD2 | ASP | B | 748 | 34.533 | 19.981 | 4.928 | 1.00 | 40.36 |
| ATOM | 4329 | C | ASP | B | 748 | 35.546 | 24.071 | 5.265 | 1.00 | 32.12 |
| ATOM | 4330 | O | ASP | B | 748 | 35.026 | 24.538 | 6.280 | 1.00 | 31.68 |
| ATOM | 4331 | N | PRO | B | 749 | 35.317 | 24.586 | 4.046 | 1.00 | 30.33 |
| ATOM | 4332 | CD | PRO | B | 749 | 35.694 | 23.972 | 2.761 | 1.00 | 44.68 |
| ATOM | 4333 | CA | PRO | B | 749 | 34.445 | 25.746 | 3.827 | 1.00 | 30.40 |
| ATOM | 4334 | CB | PRO | B | 749 | 34.380 | 25.845 | 2.304 | 1.00 | 44.80 |
| ATOM | 4335 | CG | PRO | B | 749 | 34.577 | 24.423 | 1.863 | 1.00 | 45.23 |
| ATOM | 4336 | C | PRO | B | 749 | 33.065 | 25.626 | 4.470 | 1.00 | 30.78 |
| ATOM | 4337 | O | PRO | B | 749 | 32.553 | 26.591 | 5.045 | 1.00 | 29.64 |
| ATOM | 4338 | N | HIS | B | 750 | 32.464 | 24.443 | 4.379 | 1.00 | 31.42 |
| ATOM | 4339 | CA | HIS | B | 750 | 31.148 | 24.231 | 4.963 | 1.00 | 31.75 |
| ATOM | 4340 | CB | HIS | B | 750 | 30.569 | 22.890 | 4.513 | 1.00 | 36.05 |
| ATOM | 4341 | CG | HIS | B | 750 | 29.289 | 22.529 | 5.200 | 1.00 | 36.27 |
| ATOM | 4342 | CD2 | HIS | B | 750 | 28.002 | 22.836 | 4.908 | 1.00 | 35.87 |
| ATOM | 4343 | ND1 | HIS | B | 750 | 29.250 | 21.786 | 6.360 | 1.00 | 36.16 |
| ATOM | 4344 | CE1 | HIS | B | 750 | 27.996 | 21.648 | 6.752 | 1.00 | 36.46 |
| ATOM | 4345 | NE2 | HIS | B | 750 | 27.219 | 22.276 | 5.888 | 1.00 | 36.37 |
| ATOM | 4346 | C | HIS | B | 750 | 31.212 | 24.288 | 6.480 | 1.00 | 31.05 |
| ATOM | 4347 | O | HIS | B | 750 | 30.373 | 24.922 | 7.118 | 1.00 | 31.52 |
| ATOM | 4348 | N | GLU | B | 751 | 32.208 | 23.629 | 7.062 | 1.00 | 26.46 |
| ATOM | 4349 | CA | GLU | B | 751 | 32.356 | 23.642 | 8.512 | 1.00 | 26.08 |
| ATOM | 4350 | CB | GLU | B | 751 | 33.415 | 22.625 | 8.946 | 1.00 | 32.16 |
| ATOM | 4351 | CG | GLU | B | 751 | 33.004 | 21.185 | 8.700 | 1.00 | 32.81 |
| ATOM | 4352 | CD | GLU | B | 751 | 34.040 | 20.193 | 9.190 | 1.00 | 32.54 |
| ATOM | 4353 | OE1 | GLU | B | 751 | 34.381 | 20.234 | 10.392 | 1.00 | 32.25 |
| ATOM | 4354 | OE2 | GLU | B | 751 | 34.507 | 19.373 | 8.372 | 1.00 | 33.04 |
| ATOM | 4355 | C | GLU | B | 751 | 32.741 | 25.038 | 8.997 | 1.00 | 25.23 |
| ATOM | 4356 | O | GLU | B | 751 | 32.426 | 25.424 | 10.127 | 1.00 | 26.00 |
| ATOM | 4357 | N | LYS | B | 752 | 33.413 | 25.791 | 8.134 | 1.00 | 25.46 |
| ATOM | 4358 | CA | LYS | B | 752 | 33.835 | 27.144 | 8.477 | 1.00 | 25.14 |
| ATOM | 4359 | CB | LYS | B | 752 | 34.760 | 27.710 | 7.400 | 1.00 | 34.00 |
| ATOM | 4360 | CG | LYS | B | 752 | 35.473 | 28.988 | 7.807 | 1.00 | 34.35 |
| ATOM | 4361 | CD | LYS | B | 752 | 36.445 | 28.715 | 8.950 | 1.00 | 34.98 |
| ATOM | 4362 | CE | LYS | B | 752 | 37.386 | 29.878 | 9.166 | 1.00 | 34.20 |
| ATOM | 4363 | NZ | LYS | B | 752 | 38.098 | 30.208 | 7.910 | 1.00 | 34.22 |
| ATOM | 4364 | C | LYS | B | 752 | 32.600 | 28.024 | 8.593 | 1.00 | 25.44 |
| ATOM | 4365 | O | LYS | B | 752 | 32.435 | 28.747 | 9.574 | 1.00 | 25.14 |
| ATOM | 4366 | N | GLU | B | 753 | 31.724 | 27.956 | 7.597 | 1.00 | 26.17 |
| ATOM | 4367 | CA | GLU | B | 753 | 30.519 | 28.775 | 7.639 | 1.00 | 27.32 |
| ATOM | 4368 | CB | GLU | B | 753 | 29.773 | 28.743 | 6.300 | 1.00 | 47.16 |
| ATOM | 4369 | CG | GLU | B | 753 | 29.010 | 27.468 | 6.008 | 1.00 | 48.02 |
| ATOM | 4370 | CD | GLU | B | 753 | 28.190 | 27.578 | 4.738 | 1.00 | 48.29 |
| ATOM | 4371 | OE1 | GLU | B | 753 | 28.791 | 27.692 | 3.647 | 1.00 | 48.21 |
| ATOM | 4372 | OE2 | GLU | B | 753 | 26.944 | 27.561 | 4.832 | 1.00 | 48.25 |
| ATOM | 4373 | C | GLU | B | 753 | 29.620 | 28.287 | 8.764 | 1.00 | 26.46 |
| ATOM | 4374 | O | GLU | B | 753 | 28.913 | 29.080 | 9.383 | 1.00 | 26.43 |
| ATOM | 4375 | N | LEU | B | 754 | 29.658 | 26.983 | 9.035 | 1.00 | 24.18 |
| ATOM | 4376 | CA | LEU | B | 754 | 28.860 | 26.406 | 10.110 | 1.00 | 22.29 |
| ATOM | 4377 | CB | LEU | B | 754 | 28.981 | 24.877 | 10.116 | 1.00 | 25.44 |
| ATOM | 4378 | CG | LEU | B | 754 | 28.264 | 24.173 | 11.273 | 1.00 | 25.42 |
| ATOM | 4379 | CD1 | LEU | B | 754 | 26.772 | 24.485 | 11.234 | 1.00 | 26.03 |
| ATOM | 4380 | CD2 | LEU | B | 754 | 28.494 | 22.674 | 11.174 | 1.00 | 26.23 |
| ATOM | 4381 | C | LEU | B | 754 | 29.338 | 26.957 | 11.451 | 1.00 | 22.78 |
| ATOM | 4382 | O | LEU | B | 754 | 28.524 | 27.323 | 12.308 | 1.00 | 23.06 |
| ATOM | 4383 | N | PHE | B | 755 | 30.657 | 27.015 | 11.636 | 1.00 | 20.22 |
| ATOM | 4384 | CA | PHE | B | 755 | 31.216 | 27.528 | 12.872 | 1.00 | 20.52 |
| ATOM | 4385 | CB | PHE | B | 755 | 32.745 | 27.445 | 12.870 | 1.00 | 21.28 |
| ATOM | 4386 | CG | PHE | B | 755 | 33.380 | 28.142 | 14.042 | 1.00 | 20.97 |
| ATOM | 4387 | CD1 | PHE | B | 755 | 33.212 | 27.654 | 15.332 | 1.00 | 21.53 |
| ATOM | 4388 | CD2 | PHE | B | 755 | 34.111 | 29.309 | 13.858 | 1.00 | 22.19 |
| ATOM | 4389 | CE1 | PHE | B | 755 | 33.763 | 28.323 | 16.426 | 1.00 | 21.26 |
| ATOM | 4390 | CE2 | PHE | B | 755 | 34.667 | 29.989 | 14.949 | 1.00 | 21.93 |
| ATOM | 4391 | CZ | PHE | B | 755 | 34.492 | 29.496 | 16.230 | 1.00 | 21.53 |
| ATOM | 4392 | C | PHE | B | 755 | 30.811 | 28.982 | 13.064 | 1.00 | 19.98 |
| ATOM | 4393 | O | PHE | B | 755 | 30.378 | 29.372 | 14.147 | 1.00 | 22.32 |
| ATOM | 4394 | N | LEU | B | 756 | 30.968 | 29.778 | 12.010 | 1.00 | 19.82 |
| ATOM | 4395 | CA | LEU | B | 756 | 30.621 | 31.193 | 12.070 | 1.00 | 19.75 |
| ATOM | 4396 | CB | LEU | B | 756 | 30.868 | 31.858 | 10.716 | 1.00 | 18.84 |
| ATOM | 4397 | CG | LEU | B | 756 | 32.329 | 31.854 | 10.252 | 1.00 | 18.79 |
| ATOM | 4398 | CD1 | LEU | B | 756 | 32.433 | 32.482 | 8.864 | 1.00 | 19.24 |
| ATOM | 4399 | CD2 | LEU | B | 756 | 33.192 | 32.610 | 11.262 | 1.00 | 19.62 |
| ATOM | 4400 | C | LEU | B | 756 | 29.165 | 31.372 | 12.488 | 1.00 | 21.11 |
| ATOM | 4401 | O | LEU | B | 756 | 28.839 | 32.294 | 13.233 | 1.00 | 19.94 |
| ATOM | 4402 | N | ALA | B | 757 | 28.291 | 30.488 | 12.014 | 1.00 | 20.80 |
| ATOM | 4403 | CA | ALA | B | 757 | 26.873 | 30.568 | 12.373 | 1.00 | 21.25 |
| ATOM | 4404 | CB | ALA | B | 757 | 26.064 | 29.560 | 11.552 | 1.00 | 23.40 |
| ATOM | 4405 | C | ALA | B | 757 | 26.693 | 30.294 | 13.861 | 1.00 | 22.25 |
| ATOM | 4406 | O | ALA | B | 757 | 25.917 | 30.971 | 14.542 | 1.00 | 22.17 |
| ATOM | 4407 | N | MET | B | 758 | 27.412 | 29.291 | 14.364 | 1.00 | 19.96 |
| ATOM | 4408 | CA | MET | B | 758 | 27.348 | 28.926 | 15.771 | 1.00 | 20.73 |
| ATOM | 4409 | CB | MET | B | 758 | 28.154 | 27.647 | 16.023 | 1.00 | 27.97 |
| ATOM | 4410 | CG | MET | B | 758 | 27.601 | 26.422 | 15.324 | 1.00 | 27.07 |
| ATOM | 4411 | SD | MET | B | 758 | 25.940 | 25.980 | 15.881 | 1.00 | 27.12 |
| ATOM | 4412 | CE | MET | B | 758 | 24.971 | 26.627 | 14.530 | 1.00 | 27.43 |
| ATOM | 4413 | C | MET | B | 758 | 27.895 | 30.043 | 16.656 | 1.00 | 18.82 |
| ATOM | 4414 | O | MET | B | 758 | 27.352 | 30.315 | 17.725 | 1.00 | 21.47 |
| ATOM | 4415 | N | LEU | B | 759 | 28.976 | 30.674 | 16.208 | 1.00 | 19.71 |
| ATOM | 4416 | CA | LEU | B | 759 | 29.597 | 31.756 | 16.954 | 1.00 | 18.16 |
| ATOM | 4417 | CB | LEU | B | 759 | 30.892 | 32.189 | 16.270 | 1.00 | 20.33 |
| ATOM | 4418 | CG | LEU | B | 759 | 31.646 | 33.349 | 16.919 | 1.00 | 19.85 |
| ATOM | 4419 | CD1 | LEU | B | 759 | 31.992 | 33.005 | 18.363 | 1.00 | 21.09 |
| ATOM | 4420 | CD2 | LEU | B | 759 | 32.891 | 33.646 | 16.103 | 1.00 | 19.80 |
| ATOM | 4421 | C | LEU | B | 759 | 28.635 | 32.933 | 17.048 | 1.00 | 20.05 |
| ATOM | 4422 | O | LEU | B | 759 | 28.535 | 33.577 | 18.091 | 1.00 | 19.37 |
| ATOM | 4423 | N | MET | B | 760 | 27.921 | 33.202 | 15.960 | 1.00 | 18.13 |
| ATOM | 4424 | CA | MET | B | 760 | 26.955 | 34.302 | 15.959 | 1.00 | 19.70 |
| ATOM | 4425 | CB | MET | B | 760 | 26.325 | 34.455 | 14.575 | 1.00 | 17.26 |
| ATOM | 4426 | CG | MET | B | 760 | 27.211 | 35.177 | 13.547 | 1.00 | 16.88 |
| ATOM | 4427 | SD | MET | B | 760 | 27.567 | 36.936 | 13.895 | 1.00 | 17.69 |
| ATOM | 4428 | CE | MET | B | 760 | 25.887 | 37.644 | 13.962 | 1.00 | 17.02 |
| ATOM | 4429 | C | MET | B | 760 | 25.879 | 34.059 | 17.017 | 1.00 | 18.46 |
| ATOM | 4430 | O | MET | B | 760 | 25.510 | 34.970 | 17.765 | 1.00 | 18.74 |
| ATOM | 4431 | N | THR | B | 761 | 25.380 | 32.831 | 17.102 | 1.00 | 17.88 |
| ATOM | 4432 | CA | THR | B | 761 | 24.358 | 32.516 | 18.092 | 1.00 | 17.90 |
| ATOM | 4433 | CB | THR | B | 761 | 23.858 | 31.070 | 17.933 | 1.00 | 20.06 |
| ATOM | 4434 | OG1 | THR | B | 761 | 23.316 | 30.905 | 16.619 | 1.00 | 20.01 |
| ATOM | 4435 | OG2 | THR | B | 761 | 22.778 | 30.746 | 18.971 | 1.00 | 19.13 |
| ATOM | 4436 | C | THR | B | 761 | 24.922 | 32.694 | 19.495 | 1.00 | 18.35 |
| ATOM | 4437 | O | THR | B | 761 | 24.265 | 33.241 | 20.375 | 1.00 | 17.84 |
| ATOM | 4438 | N | ALA | B | 762 | 26.160 | 32.244 | 19.695 | 1.00 | 18.42 |
| ATOM | 4439 | CA | ALA | B | 762 | 26.808 | 32.347 | 20.996 | 1.00 | 17.98 |
| ATOM | 4440 | CB | ALA | B | 762 | 28.224 | 31.780 | 20.930 | 1.00 | 19.48 |
| ATOM | 4441 | C | ALA | B | 762 | 26.843 | 33.795 | 21.472 | 1.00 | 17.59 |
| ATOM | 4442 | O | ALA | B | 762 | 26.567 | 34.077 | 22.637 | 1.00 | 20.01 |
| ATOM | 4443 | N | CYS | B | 763 | 27.186 | 34.705 | 20.570 | 1.00 | 17.78 |
| ATOM | 4444 | CA | CYS | B | 763 | 27.247 | 36.115 | 20.914 | 1.00 | 18.49 |
| ATOM | 4445 | CB | CYS | B | 763 | 28.041 | 36.869 | 19.850 | 1.00 | 20.27 |
| ATOM | 4446 | SG | CYS | B | 763 | 29.790 | 36.353 | 19.790 | 1.00 | 21.15 |
| ATOM | 4447 | C | CYS | B | 763 | 25.841 | 36.704 | 21.071 | 1.00 | 19.17 |
| ATOM | 4448 | O | CYS | B | 763 | 25.596 | 37.521 | 21.957 | 1.00 | 19.53 |
| ATOM | 4449 | N | ASP | B | 764 | 24.921 | 36.265 | 20.219 | 1.00 | 20.39 |
| ATOM | 4450 | CA | ASP | B | 764 | 23.534 | 36.729 | 20.254 | 1.00 | 20.66 |
| ATOM | 4451 | CB | ASP | B | 764 | 22.739 | 36.046 | 19.130 | 1.00 | 19.31 |
| ATOM | 4452 | CG | ASP | B | 764 | 21.474 | 36.802 | 18.767 | 1.00 | 19.31 |
| ATOM | 4453 | OD1 | ASP | B | 764 | 20.707 | 36.320 | 17.903 | 1.00 | 19.87 |
| ATOM | 4454 | OD2 | ASP | B | 764 | 21.265 | 37.889 | 19.341 | 1.00 | 17.76 |
| ATOM | 4455 | C | ASP | B | 764 | 22.862 | 36.429 | 21.598 | 1.00 | 20.80 |
| ATOM | 4456 | O | ASP | B | 764 | 22.021 | 37.191 | 22.074 | 1.00 | 20.53 |
| ATOM | 4457 | N | LEU | B | 765 | 23.235 | 35.319 | 22.220 | 1.00 | 17.19 |
| ATOM | 4458 | CA | LEU | B | 765 | 22.626 | 34.934 | 23.489 | 1.00 | 17.73 |
| ATOM | 4459 | CB | LEU | B | 765 | 22.337 | 33.428 | 23.476 | 1.00 | 20.86 |
| ATOM | 4460 | CG | LEU | B | 765 | 21.556 | 32.898 | 22.270 | 1.00 | 21.66 |
| ATOM | 4461 | CD1 | LEU | B | 765 | 21.264 | 31.422 | 22.444 | 1.00 | 21.03 |
| ATOM | 4462 | CD2 | LEU | B | 765 | 20.257 | 33.679 | 22.125 | 1.00 | 20.47 |
| ATOM | 4463 | C | LEU | B | 765 | 23.497 | 35.276 | 24.697 | 1.00 | 18.18 |
| ATOM | 4464 | O | LEU | B | 765 | 23.205 | 34.833 | 25.813 | 1.00 | 19.59 |
| ATOM | 4465 | N | SER | B | 766 | 24.516 | 36.111 | 24.475 | 1.00 | 19.68 |
| ATOM | 4466 | CA | SER | B | 766 | 25.498 | 36.475 | 25.504 | 1.00 | 20.44 |
| ATOM | 4467 | CB | SER | B | 766 | 26.478 | 37.527 | 24.959 | 1.00 | 23.53 |
| ATOM | 4468 | OG | SER | B | 766 | 25.937 | 38.837 | 24.998 | 1.00 | 25.78 |
| ATOM | 4469 | C | SER | B | 766 | 24.997 | 36.932 | 26.872 | 1.00 | 20.03 |
| ATOM | 4470 | O | SER | B | 766 | 25.695 | 36.770 | 27.871 | 1.00 | 19.65 |
| ATOM | 4471 | N | ALA | B | 767 | 23.806 | 37.513 | 26.929 | 1.00 | 22.57 |
| ATOM | 4472 | CA | ALA | B | 767 | 23.284 | 37.974 | 28.210 | 1.00 | 23.91 |
| ATOM | 4473 | CB | ALA | B | 767 | 21.943 | 38.682 | 28.002 | 1.00 | 17.04 |
| ATOM | 4474 | C | ALA | B | 767 | 23.123 | 36.832 | 29.213 | 1.00 | 23.55 |
| ATOM | 4475 | O | ALA | B | 767 | 23.202 | 37.045 | 30.426 | 1.00 | 24.64 |
| ATOM | 4476 | N | ILE | B | 768 | 22.911 | 35.621 | 28.703 | 1.00 | 23.20 |
| ATOM | 4477 | CA | ILE | B | 768 | 22.718 | 34.448 | 29.546 | 1.00 | 22.31 |
| ATOM | 4478 | CB | ILE | B | 768 | 22.143 | 33.257 | 28.717 | 1.00 | 23.65 |
| ATOM | 4479 | CG2 | ILE | B | 768 | 23.253 | 32.563 | 27.933 | 1.00 | 22.89 |
| ATOM | 4480 | CG1 | ILE | B | 768 | 21.457 | 32.255 | 29.641 | 1.00 | 23.26 |
| ATOM | 4481 | CD1 | ILE | B | 768 | 20.179 | 32.796 | 30.272 | 1.00 | 23.73 |
| ATOM | 4482 | C | ILE | B | 768 | 24.004 | 33.999 | 30.247 | 1.00 | 22.60 |
| ATOM | 4483 | O | ILE | B | 768 | 23.958 | 33.164 | 31.140 | 1.00 | 22.93 |
| ATOM | 4484 | N | THR | B | 769 | 25.138 | 34.571 | 29.849 | 1.00 | 21.92 |
| ATOM | 4485 | CA | THR | B | 769 | 26.438 | 34.224 | 30.435 | 1.00 | 21.95 |
| ATOM | 4486 | CB | THR | B | 769 | 27.525 | 34.077 | 29.356 | 1.00 | 23.33 |
| ATOM | 4487 | OG1 | THR | B | 769 | 27.793 | 35.362 | 28.778 | 1.00 | 24.40 |
| ATOM | 4488 | CG2 | THR | B | 769 | 27.087 | 33.111 | 28.267 | 1.00 | 24.02 |
| ATOM | 4489 | C | THR | B | 769 | 26.962 | 35.287 | 31.399 | 1.00 | 22.67 |
| ATOM | 4490 | O | THR | B | 769 | 27.973 | 35.086 | 32.073 | 1.00 | 22.65 |
| ATOM | 4491 | N | LYS | B | 770 | 26.284 | 36.425 | 31.452 | 1.00 | 23.25 |
| ATOM | 4492 | CA | LYS | B | 770 | 26.720 | 37.530 | 32.296 | 1.00 | 23.60 |
| ATOM | 4493 | CB | LYS | B | 770 | 25.996 | 38.802 | 31.849 | 1.00 | 21.12 |
| ATOM | 4494 | CG | LYS | B | 770 | 26.258 | 39.179 | 30.403 | 1.00 | 20.96 |
| ATOM | 4495 | CD | LYS | B | 770 | 27.656 | 39.759 | 30.206 | 1.00 | 20.82 |
| ATOM | 4496 | CE | LYS | B | 770 | 27.893 | 40.115 | 28.739 | 1.00 | 20.75 |
| ATOM | 4497 | NZ | LYS | B | 770 | 29.176 | 40.840 | 28.511 | 1.00 | 21.62 |
| ATOM | 4498 | C | LYS | B | 770 | 26.525 | 37.339 | 33.802 | 1.00 | 24.02 |
| ATOM | 4499 | O | LYS | B | 770 | 25.758 | 36.476 | 34.239 | 1.00 | 23.96 |
| ATOM | 4500 | N | PRO | B | 771 | 27.238 | 38.140 | 34.618 | 1.00 | 24.80 |
| ATOM | 4501 | CD | PRO | B | 771 | 28.330 | 39.060 | 34.249 | 1.00 | 25.87 |
| ATOM | 4502 | CA | PRO | B | 771 | 27.109 | 38.045 | 36.076 | 1.00 | 25.95 |
| ATOM | 4503 | CB | PRO | B | 771 | 27.976 | 39.198 | 36.569 | 1.00 | 25.81 |
| ATOM | 4504 | CG | PRO | B | 771 | 29.083 | 39.215 | 35.563 | 1.00 | 26.51 |
| ATOM | 4505 | C | PRO | B | 771 | 25.631 | 38.220 | 36.440 | 1.00 | 26.70 |
| ATOM | 4506 | O | PRO | B | 771 | 24.905 | 38.955 | 35.763 | 1.00 | 25.64 |
| ATOM | 4507 | N | TRP | B | 772 | 25.202 | 37.546 | 37.503 | 1.00 | 27.13 |
| ATOM | 4508 | CA | TRP | B | 772 | 23.811 | 37.553 | 37.959 | 1.00 | 27.52 |
| ATOM | 4509 | CB | TRP | B | 772 | 23.744 | 37.021 | 39.394 | 1.00 | 31.16 |
| ATOM | 4510 | CG | TRP | B | 772 | 22.355 | 36.817 | 39.921 | 1.00 | 31.91 |
| ATOM | 4511 | CD2 | TRP | B | 772 | 21.272 | 36.141 | 39.267 | 1.00 | 31.55 |
| ATOM | 4512 | CE2 | TRP | B | 772 | 20.171 | 36.166 | 40.150 | 1.00 | 31.84 |
| ATOM | 4513 | CE3 | TRP | B | 772 | 21.126 | 35.516 | 38.021 | 1.00 | 31.35 |
| ATOM | 4514 | CD1 | TRP | B | 772 | 21.878 | 37.212 | 41.138 | 1.00 | 31.40 |
| ATOM | 4515 | NE1 | TRP | B | 772 | 20.569 | 36.827 | 41.283 | 1.00 | 32.07 |
| ATOM | 4516 | CZ2 | TRP | B | 772 | 18.937 | 35.591 | 39.828 | 1.00 | 32.13 |
| ATOM | 4517 | CZ3 | TRP | B | 772 | 19.897 | 34.944 | 37.701 | 1.00 | 31.80 |
| ATOM | 4518 | CH2 | TRP | B | 772 | 18.819 | 34.987 | 38.602 | 1.00 | 31.93 |
| ATOM | 4519 | C | TRP | B | 772 | 23.009 | 38.853 | 37.865 | 1.00 | 27.06 |
| ATOM | 4520 | O | TRP | B | 772 | 21.913 | 38.864 | 37.305 | 1.00 | 27.10 |
| ATOM | 4521 | N | PRO | B | 773 | 23.533 | 39.964 | 38.409 | 1.00 | 27.24 |
| ATOM | 4522 | CD | PRO | B | 773 | 24.841 | 40.176 | 39.050 | 1.00 | 36.67 |
| ATOM | 4523 | CA | PRO | B | 773 | 22.778 | 41.220 | 38.336 | 1.00 | 26.63 |
| ATOM | 4524 | CB | PRO | B | 773 | 23.649 | 42.192 | 39.133 | 1.00 | 36.87 |
| ATOM | 4525 | CG | PRO | B | 773 | 25.027 | 41.669 | 38.902 | 1.00 | 37.42 |
| ATOM | 4526 | C | PRO | B | 773 | 22.520 | 41.692 | 36.914 | 1.00 | 25.67 |
| ATOM | 4527 | O | PRO | B | 773 | 21.476 | 42.272 | 36.626 | 1.00 | 26.52 |
| ATOM | 4528 | N | ILE | B | 774 | 23.480 | 41.443 | 36.029 | 1.00 | 21.82 |
| ATOM | 4529 | CA | ILE | B | 774 | 23.341 | 41.834 | 34.636 | 1.00 | 21.26 |
| ATOM | 4530 | CB | ILE | B | 774 | 24.707 | 41.788 | 33.915 | 1.00 | 30.27 |
| ATOM | 4531 | CG2 | ILE | B | 774 | 24.523 | 42.062 | 32.429 | 1.00 | 30.62 |
| ATOM | 4532 | CG1 | ILE | B | 774 | 25.649 | 42.821 | 34.543 | 1.00 | 31.27 |
| ATOM | 4533 | CD1 | ILE | B | 774 | 27.075 | 42.765 | 34.037 | 1.00 | 31.12 |
| ATOM | 4534 | C | ILE | B | 774 | 22.342 | 40.909 | 33.947 | 1.00 | 20.74 |
| ATOM | 4535 | O | ILE | B | 774 | 21.452 | 41.375 | 33.239 | 1.00 | 21.33 |
| ATOM | 4536 | N | GLN | B | 775 | 22.482 | 39.604 | 34.166 | 1.00 | 22.90 |
| ATOM | 4537 | CA | GLN | B | 775 | 21.569 | 38.636 | 33.565 | 1.00 | 23.62 |
| ATOM | 4538 | CB | GLN | B | 775 | 21.965 | 37.205 | 33.956 | 1.00 | 24.73 |
| ATOM | 4539 | CG | GLN | B | 775 | 20.801 | 36.214 | 33.995 | 1.00 | 25.27 |
| ATOM | 4540 | CD | GLN | B | 775 | 20.070 | 36.062 | 32.661 | 1.00 | 25.01 |
| ATOM | 4541 | OE1 | GLN | B | 775 | 18.983 | 35.495 | 32.610 | 1.00 | 23.97 |
| ATOM | 4542 | NE2 | GLN | B | 775 | 20.669 | 36.556 | 31.583 | 1.00 | 25.73 |
| ATOM | 4543 | C | GLN | B | 775 | 20.127 | 38.905 | 33.987 | 1.00 | 24.69 |
| ATOM | 4544 | O | GLN | B | 775 | 19.213 | 38.883 | 33.161 | 1.00 | 23.74 |
| ATOM | 4545 | N | GLN | B | 776 | 19.930 | 39.158 | 35.277 | 1.00 | 24.42 |
| ATOM | 4546 | CA | GLN | B | 776 | 18.596 | 39.440 | 35.800 | 1.00 | 25.46 |
| ATOM | 4547 | CB | GLN | B | 776 | 18.647 | 39.710 | 37.303 | 1.00 | 39.22 |
| ATOM | 4548 | CG | GLN | B | 776 | 18.627 | 38.482 | 38.172 | 1.00 | 40.03 |
| ATOM | 4549 | CD | GLN | B | 776 | 18.641 | 38.828 | 39.648 | 1.00 | 40.44 |
| ATOM | 4550 | OE1 | GLN | B | 776 | 19.635 | 39.339 | 40.167 | 1.00 | 40.18 |
| ATOM | 4551 | NE2 | GLN | B | 776 | 17.536 | 38.554 | 40.332 | 1.00 | 40.01 |
| ATOM | 4552 | C | GLN | B | 776 | 17.989 | 40.658 | 35.121 | 1.00 | 24.70 |
| ATOM | 4553 | O | GLN | B | 776 | 16.832 | 40.641 | 34.712 | 1.00 | 25.09 |
| ATOM | 4554 | N | ARG | B | 777 | 18.773 | 41.725 | 35.028 | 1.00 | 23.31 |
| ATOM | 4555 | CA | ARG | B | 777 | 18.298 | 42.949 | 34.410 | 1.00 | 23.89 |
| ATOM | 4556 | CB | ARG | B | 777 | 19.369 | 44.038 | 34.481 | 1.00 | 27.39 |
| ATOM | 4557 | CG | ARG | B | 777 | 18.902 | 45.369 | 33.917 | 1.00 | 27.72 |
| ATOM | 4558 | CD | ARG | B | 777 | 19.965 | 46.434 | 34.058 | 1.00 | 28.34 |
| ATOM | 4559 | NE | ARG | B | 777 | 21.102 | 46.209 | 33.170 | 1.00 | 27.57 |
| ATOM | 4560 | CZ | ARG | B | 777 | 21.029 | 46.232 | 31.843 | 1.00 | 27.93 |
| ATOM | 4561 | NH1 | ARG | B | 777 | 19.870 | 46.468 | 31.244 | 1.00 | 28.38 |
| ATOM | 4562 | NH2 | ARG | B | 777 | 22.119 | 46.032 | 31.113 | 1.00 | 27.27 |
| ATOM | 4563 | C | ARG | B | 777 | 17.905 | 42.728 | 32.958 | 1.00 | 23.38 |
| ATOM | 4564 | O | ARG | B | 777 | 16.814 | 43.119 | 32.535 | 1.00 | 24.57 |
| ATOM | 4565 | N | ILE | B | 778 | 18.784 | 42.089 | 32.191 | 1.00 | 18.64 |
| ATOM | 4566 | CA | ILE | B | 778 | 18.487 | 41.853 | 30.783 | 1.00 | 19.91 |
| ATOM | 4567 | CB | ILE | B | 778 | 19.729 | 41.350 | 30.028 | 1.00 | 21.48 |
| ATOM | 4568 | CG2 | ILE | B | 778 | 19.371 | 41.065 | 28.575 | 1.00 | 21.88 |
| ATOM | 4569 | CG1 | ILE | B | 778 | 20.809 | 42.432 | 30.077 | 1.00 | 22.67 |
| ATOM | 4570 | CD1 | ILE | B | 778 | 22.127 | 42.024 | 29.472 | 1.00 | 22.44 |
| ATOM | 4571 | C | ILE | B | 778 | 17.333 | 40.880 | 30.605 | 1.00 | 19.30 |
| ATOM | 4572 | O | ILE | B | 778 | 16.494 | 41.063 | 29.718 | 1.00 | 18.16 |
| ATOM | 4573 | N | ALA | B | 779 | 17.272 | 39.865 | 31.459 | 1.00 | 20.27 |
| ATOM | 4574 | CA | ALA | B | 779 | 16.193 | 38.890 | 31.393 | 1.00 | 21.96 |
| ATOM | 4575 | CB | ALA | B | 779 | 16.333 | 37.880 | 32.513 | 1.00 | 23.30 |
| ATOM | 4576 | C | ALA | B | 779 | 14.851 | 39.610 | 31.495 | 1.00 | 21.94 |
| ATOM | 4577 | O | ALA | B | 779 | 13.896 | 39.271 | 30.790 | 1.00 | 21.55 |
| ATOM | 4578 | N | GLU | B | 780 | 14.780 | 40.615 | 32.361 | 1.00 | 23.66 |
| ATOM | 4579 | CA | GLU | B | 780 | 13.539 | 41.368 | 32.520 | 1.00 | 24.79 |
| ATOM | 4580 | CB | GLU | B | 780 | 13.643 | 42.309 | 33.723 | 1.00 | 46.99 |
| ATOM | 4581 | CG | GLU | B | 780 | 13.971 | 41.572 | 35.016 | 1.00 | 47.98 |
| ATOM | 4582 | CD | GLU | B | 780 | 13.473 | 42.287 | 36.255 | 1.00 | 48.28 |
| ATOM | 4583 | OE1 | GLU | B | 780 | 13.748 | 43.499 | 36.398 | 1.00 | 48.22 |
| ATOM | 4584 | OE2 | GLU | B | 780 | 12.814 | 41.630 | 37.089 | 1.00 | 48.29 |
| ATOM | 4585 | C | GLU | B | 780 | 13.171 | 42.144 | 31.251 | 1.00 | 23.73 |
| ATOM | 4586 | O | GLU | B | 780 | 11.989 | 42.308 | 30.931 | 1.00 | 23.36 |
| ATOM | 4587 | N | LEU | B | 781 | 14.180 | 42.615 | 30.525 | 1.00 | 21.19 |
| ATOM | 4588 | CA | LEU | B | 781 | 13.949 | 43.336 | 29.282 | 1.00 | 19.63 |
| ATOM | 4589 | CB | LEU | B | 781 | 15.257 | 43.926 | 28.761 | 1.00 | 24.73 |
| ATOM | 4590 | CG | LEU | B | 781 | 15.902 | 44.956 | 29.687 | 1.00 | 25.34 |
| ATOM | 4591 | CD1 | LEU | B | 781 | 17.189 | 45.469 | 29.065 | 1.00 | 25.01 |
| ATOM | 4592 | CD2 | LEU | B | 781 | 14.926 | 46.101 | 29.913 | 1.00 | 24.85 |
| ATOM | 4593 | C | LEU | B | 781 | 13.393 | 42.353 | 28.261 | 1.00 | 21.36 |
| ATOM | 4594 | O | LEU | B | 781 | 12.510 | 42.686 | 27.466 | 1.00 | 20.39 |
| ATOM | 4595 | N | VAL | B | 782 | 13.918 | 41.134 | 28.286 | 1.00 | 21.98 |
| ATOM | 4596 | CA | VAL | B | 782 | 13.455 | 40.112 | 27.360 | 1.00 | 22.60 |
| ATOM | 4597 | CB | VAL | B | 782 | 14.275 | 38.812 | 27.499 | 1.00 | 22.53 |
| ATOM | 4598 | CG1 | VAL | B | 782 | 13.660 | 37.713 | 26.643 | 1.00 | 22.91 |
| ATOM | 4599 | CG2 | VAL | B | 782 | 15.712 | 39.056 | 27.054 | 1.00 | 23.90 |
| ATOM | 4600 | C | VAL | B | 782 | 11.988 | 39.808 | 27.648 | 1.00 | 22.59 |
| ATOM | 4601 | O | VAL | B | 782 | 11.159 | 39.746 | 26.737 | 1.00 | 22.68 |
| ATOM | 4602 | N | ALA | B | 783 | 11.671 | 39.628 | 28.925 | 1.00 | 20.36 |
| ATOM | 4603 | CA | ALA | B | 783 | 10.301 | 39.327 | 29.323 | 1.00 | 20.69 |
| ATOM | 4604 | CB | ALA | B | 783 | 10.232 | 39.082 | 30.821 | 1.00 | 21.72 |
| ATOM | 4605 | C | ALA | B | 783 | 9.376 | 40.474 | 28.932 | 1.00 | 20.76 |
| ATOM | 4606 | O | ALA | B | 783 | 8.283 | 40.249 | 28.426 | 1.00 | 22.62 |
| ATOM | 4607 | N | THR | B | 784 | 9.823 | 41.704 | 29.155 | 1.00 | 22.97 |
| ATOM | 4608 | CA | THR | B | 784 | 9.013 | 42.862 | 28.807 | 1.00 | 23.28 |
| ATOM | 4609 | CB | THR | B | 784 | 9.752 | 44.182 | 29.139 | 1.00 | 23.34 |
| ATOM | 4610 | OG1 | THR | B | 784 | 9.934 | 44.280 | 30.562 | 1.00 | 23.54 |
| ATOM | 4611 | CG2 | THR | B | 784 | 8.956 | 45.387 | 28.645 | 1.00 | 23.16 |
| ATOM | 4612 | C | THR | B | 784 | 8.643 | 42.846 | 27.322 | 1.00 | 23.86 |
| ATOM | 4613 | O | THR | B | 784 | 7.485 | 43.086 | 26.963 | 1.00 | 24.06 |
| ATOM | 4614 | N | GLU | B | 785 | 9.612 | 42.547 | 26.459 | 1.00 | 20.32 |
| ATOM | 4615 | CA | GLU | B | 785 | 9.333 | 42.519 | 25.029 | 1.00 | 19.62 |
| ATOM | 4616 | CB | GLU | B | 785 | 10.633 | 42.371 | 24.221 | 1.00 | 19.82 |
| ATOM | 4617 | CG | GLU | B | 785 | 10.424 | 42.346 | 22.703 | 1.00 | 19.80 |
| ATOM | 4618 | CD | GLU | B | 785 | 11.708 | 42.572 | 21.908 | 1.00 | 22.07 |
| ATOM | 4619 | OE1 | GLU | B | 785 | 11.800 | 42.066 | 20.765 | 1.00 | 20.56 |
| ATOM | 4620 | OE2 | GLU | B | 785 | 12.620 | 43.264 | 22.408 | 1.00 | 19.00 |
| ATOM | 4621 | C | GLU | B | 785 | 8.360 | 41.386 | 24.709 | 1.00 | 20.53 |
| ATOM | 4622 | O | GLU | B | 785 | 7.395 | 41.588 | 23.964 | 1.00 | 20.48 |
| ATOM | 4623 | N | PHE | B | 786 | 8.603 | 40.205 | 25.276 | 1.00 | 19.27 |
| ATOM | 4624 | CA | PHE | B | 786 | 7.725 | 39.053 | 25.054 | 1.00 | 20.35 |
| ATOM | 4625 | CB | PHE | B | 786 | 8.185 | 37.824 | 25.853 | 1.00 | 27.64 |
| ATOM | 4626 | CG | PHE | B | 786 | 9.394 | 37.122 | 25.285 | 1.00 | 28.61 |
| ATOM | 4627 | CD1 | PHE | B | 786 | 9.799 | 37.330 | 23.971 | 1.00 | 28.46 |
| ATOM | 4628 | CD2 | PHE | B | 786 | 10.111 | 36.225 | 26.072 | 1.00 | 28.06 |
| ATOM | 4629 | CE1 | PHE | B | 786 | 10.903 | 36.652 | 23.451 | 1.00 | 28.92 |
| ATOM | 4630 | CE2 | PHE | B | 786 | 11.212 | 35.543 | 25.561 | 1.00 | 28.43 |
| ATOM | 4631 | CZ | PHE | B | 786 | 11.608 | 35.758 | 24.250 | 1.00 | 28.30 |
| ATOM | 4632 | C | PHE | B | 786 | 6.289 | 39.380 | 25.475 | 1.00 | 20.75 |
| ATOM | 4633 | O | PHE | B | 786 | 5.340 | 39.061 | 24.760 | 1.00 | 18.68 |
| ATOM | 4634 | N | PHE | B | 787 | 6.144 | 40.003 | 26.646 | 1.00 | 22.35 |
| ATOM | 4635 | CA | PHE | B | 787 | 4.825 | 40.361 | 27.160 | 1.00 | 21.89 |
| ATOM | 4636 | CB | PHE | B | 787 | 4.917 | 40.801 | 28.632 | 1.00 | 21.52 |
| ATOM | 4637 | CG | PHE | B | 787 | 5.419 | 39.729 | 29.567 | 1.00 | 22.41 |
| ATOM | 4638 | CD1 | PHE | B | 787 | 5.256 | 38.376 | 29.265 | 1.00 | 22.32 |
| ATOM | 4639 | CD2 | PHE | B | 787 | 6.035 | 40.070 | 30.771 | 1.00 | 21.61 |
| ATOM | 4640 | CE1 | PHE | B | 787 | 5.698 | 37.384 | 30.148 | 1.00 | 22.73 |
| ATOM | 4641 | CE2 | PHE | B | 787 | 6.478 | 39.085 | 31.653 | 1.00 | 22.56 |
| ATOM | 4642 | CZ | PHE | B | 787 | 6.309 | 37.742 | 31.341 | 1.00 | 21.88 |
| ATOM | 4643 | C | PHE | B | 787 | 4.154 | 41.453 | 26.317 | 1.00 | 21.60 |
| ATOM | 4644 | O | PHE | B | 787 | 2.924 | 41.484 | 26.195 | 1.00 | 21.58 |
| ATOM | 4645 | N | ASP | B | 788 | 4.950 | 42.338 | 25.721 | 1.00 | 20.66 |
| ATOM | 4646 | CA | ASP | B | 788 | 4.387 | 43.384 | 24.872 | 1.00 | 21.38 |
| ATOM | 4647 | CB | ASP | B | 788 | 5.459 | 44.404 | 24.467 | 1.00 | 29.62 |
| ATOM | 4648 | CG | ASP | B | 788 | 4.892 | 45.559 | 23.646 | 1.00 | 31.03 |
| ATOM | 4649 | OD1 | ASP | B | 788 | 4.010 | 46.284 | 24.159 | 1.00 | 31.00 |
| ATOM | 4650 | OD2 | ASP | B | 788 | 5.327 | 45.750 | 22.486 | 1.00 | 30.30 |
| ATOM | 4651 | C | ASP | B | 788 | 3.802 | 42.712 | 23.630 | 1.00 | 21.98 |
| ATOM | 4652 | O | ASP | B | 788 | 2.754 | 43.116 | 23.138 | 1.00 | 20.90 |
| ATOM | 4653 | N | GLN | B | 789 | 4.480 | 41.686 | 23.117 | 1.00 | 18.78 |
| ATOM | 4654 | CA | GLN | B | 789 | 3.950 | 40.980 | 21.958 | 1.00 | 19.35 |
| ATOM | 4655 | CB | GLN | B | 789 | 4.947 | 39.965 | 21.397 | 1.00 | 18.41 |
| ATOM | 4656 | CG | GLN | B | 789 | 4.283 | 39.057 | 20.363 | 1.00 | 19.65 |
| ATOM | 4657 | CD | GLN | B | 789 | 5.228 | 38.067 | 19.715 | 1.00 | 19.91 |
| ATOM | 4658 | OE1 | GLN | B | 789 | 4.805 | 37.004 | 19.253 | 1.00 | 20.58 |
| ATOM | 4659 | NE2 | GLN | B | 789 | 6.503 | 38.413 | 19.663 | 1.00 | 17.85 |
| ATOM | 4660 | C | GLN | B | 789 | 2.690 | 40.240 | 22.382 | 1.00 | 19.10 |
| ATOM | 4661 | O | GLN | B | 789 | 1.728 | 40.154 | 21.622 | 1.00 | 19.13 |
| ATOM | 4662 | N | GLY | B | 790 | 2.713 | 39.701 | 23.598 | 1.00 | 20.13 |
| ATOM | 4663 | CA | GLY | B | 790 | 1.565 | 38.983 | 24.118 | 1.00 | 21.62 |
| ATOM | 4664 | C | GLY | B | 790 | 0.338 | 39.870 | 24.089 | 1.00 | 21.39 |
| ATOM | 4665 | O | GLY | B | 790 | −0.748 | 39.426 | 23.711 | 1.00 | 20.51 |
| ATOM | 4666 | N | ASP | B | 791 | 0.506 | 41.127 | 24.491 | 1.00 | 21.64 |
| ATOM | 4667 | CA | ASP | B | 791 | −0.612 | 42.066 | 24.488 | 1.00 | 23.10 |
| ATOM | 4668 | CB | ASP | B | 791 | −0.205 | 43.401 | 25.128 | 1.00 | 27.91 |
| ATOM | 4669 | CG | ASP | B | 791 | 0.161 | 43.263 | 26.599 | 1.00 | 28.04 |
| ATOM | 4670 | OD1 | ASP | B | 791 | −0.368 | 42.352 | 27.271 | 1.00 | 28.51 |
| ATOM | 4671 | OD2 | ASP | B | 791 | 0.967 | 44.083 | 27.092 | 1.00 | 29.30 |
| ATOM | 4672 | C | ASP | B | 791 | −1.118 | 42.307 | 23.061 | 1.00 | 22.57 |
| ATOM | 4673 | O | ASP | B | 791 | −2.328 | 42.422 | 22.837 | 1.00 | 22.71 |
| ATOM | 4674 | N | ARG | B | 792 | −0.196 | 42.388 | 22.103 | 1.00 | 22.55 |
| ATOM | 4675 | CA | ARG | B | 792 | −0.557 | 42.600 | 20.701 | 1.00 | 21.86 |
| ATOM | 4676 | CB | ARG | B | 792 | 0.697 | 42.794 | 19.831 | 1.00 | 25.58 |
| ATOM | 4677 | CG | ARG | B | 792 | 1.559 | 44.015 | 20.155 | 1.00 | 26.82 |
| ATOM | 4678 | CD | ARG | B | 792 | 1.268 | 45.192 | 19.225 | 1.00 | 27.03 |
| ATOM | 4679 | NE | ARG | B | 792 | 1.602 | 44.929 | 17.819 | 1.00 | 26.18 |
| ATOM | 4680 | CZ | ARG | B | 792 | 2.768 | 45.220 | 17.247 | 1.00 | 26.00 |
| ATOM | 4681 | NH1 | ARG | B | 792 | 3.746 | 45.785 | 17.950 | 1.00 | 26.11 |
| ATOM | 4682 | NH2 | ARG | B | 792 | 2.943 | 44.982 | 15.954 | 1.00 | 27.12 |
| ATOM | 4683 | C | ARG | B | 792 | −1.338 | 41.393 | 20.179 | 1.00 | 21.59 |
| ATOM | 4684 | O | ARG | B | 792 | −2.251 | 41.541 | 19.366 | 1.00 | 22.04 |
| ATOM | 4685 | N | GLU | B | 793 | −0.981 | 40.199 | 20.640 | 1.00 | 21.27 |
| ATOM | 4686 | CA | GLU | B | 793 | −1.678 | 38.999 | 20.189 | 1.00 | 23.41 |
| ATOM | 4687 | CB | GLU | B | 793 | −0.915 | 37.741 | 20.613 | 1.00 | 32.13 |
| ATOM | 4688 | CG | GLU | B | 793 | 0.523 | 37.716 | 20.126 | 1.00 | 32.18 |
| ATOM | 4689 | CD | GLU | B | 793 | 1.199 | 36.378 | 20.354 | 1.00 | 32.76 |
| ATOM | 4690 | OE1 | GLU | B | 793 | 0.883 | 35.713 | 21.363 | 1.00 | 33.25 |
| ATOM | 4691 | OE2 | GLU | B | 793 | 2.058 | 35.998 | 19.531 | 1.00 | 32.81 |
| ATOM | 4692 | C | GLU | B | 793 | −3.106 | 38.946 | 20.726 | 1.00 | 23.75 |
| ATOM | 4693 | O | GLU | B | 793 | −4.044 | 38.610 | 19.995 | 1.00 | 22.66 |
| ATOM | 4694 | N | ARG | B | 794 | −3.277 | 39.283 | 21.999 | 1.00 | 24.04 |
| ATOM | 4695 | CA | ARG | B | 794 | −4.603 | 39.256 | 22.599 | 1.00 | 24.34 |
| ATOM | 4696 | CB | ARG | B | 794 | −4.564 | 39.780 | 24.038 | 1.00 | 32.14 |
| ATOM | 4697 | CG | ARG | B | 794 | −3.332 | 39.401 | 24.824 | 1.00 | 33.26 |
| ATOM | 4698 | CD | ARG | B | 794 | −3.543 | 39.603 | 26.316 | 1.00 | 33.20 |
| ATOM | 4699 | NE | ARG | B | 794 | −4.342 | 38.518 | 26.866 | 1.00 | 33.66 |
| ATOM | 4700 | CZ | ARG | B | 794 | −3.964 | 37.749 | 27.881 | 1.00 | 33.17 |
| ATOM | 4701 | NH1 | ARG | B | 794 | −2.794 | 37.948 | 28.472 | 1.00 | 33.10 |
| ATOM | 4702 | NH2 | ARG | B | 794 | −4.748 | 36.759 | 28.285 | 1.00 | 33.54 |
| ATOM | 4703 | C | ARG | B | 794 | −5.566 | 40.119 | 21.797 | 1.00 | 25.06 |
| ATOM | 4704 | O | ARG | B | 794 | −6.713 | 39.740 | 21.563 | 1.00 | 24.85 |
| ATOM | 4705 | N | LYS | B | 795 | −5.090 | 41.279 | 21.369 | 1.00 | 26.49 |
| ATOM | 4706 | CA | LYS | B | 795 | −5.928 | 42.205 | 20.627 | 1.00 | 27.67 |
| ATOM | 4707 | CB | LYS | B | 795 | −5.412 | 43.633 | 20.821 | 1.00 | 43.32 |
| ATOM | 4708 | CG | LYS | B | 795 | −5.021 | 43.940 | 22.260 | 1.00 | 43.98 |
| ATOM | 4709 | CD | LYS | B | 795 | −4.846 | 45.433 | 22.504 | 1.00 | 44.35 |
| ATOM | 4710 | CE | LYS | B | 795 | −6.121 | 46.059 | 23.052 | 1.00 | 44.46 |
| ATOM | 4711 | NZ | LYS | B | 795 | −7.293 | 45.855 | 22.159 | 1.00 | 44.56 |
| ATOM | 4712 | C | LYS | B | 795 | −6.032 | 41.883 | 19.140 | 1.00 | 27.05 |
| ATOM | 4713 | O | LYS | B | 795 | −7.125 | 41.624 | 18.636 | 1.00 | 28.54 |
| ATOM | 4714 | N | GLU | B | 796 | −4.894 | 41.885 | 18.451 | 1.00 | 25.18 |
| ATOM | 4715 | CA | GLU | B | 796 | −4.842 | 41.634 | 17.007 | 1.00 | 24.55 |
| ATOM | 4716 | CB | GLU | B | 796 | −3.464 | 42.009 | 16.464 | 1.00 | 28.07 |
| ATOM | 4717 | CG | GLU | B | 796 | −2.991 | 43.391 | 16.889 | 1.00 | 29.00 |
| ATOM | 4718 | CD | GLU | B | 796 | −1.615 | 43.741 | 16.348 | 1.00 | 28.65 |
| ATOM | 4719 | OE1 | GLU | B | 796 | −0.962 | 44.626 | 16.931 | 1.00 | 29.39 |
| ATOM | 4720 | OE2 | GLU | B | 796 | −1.187 | 43.144 | 15.340 | 1.00 | 29.53 |
| ATOM | 4721 | C | GLU | B | 796 | −5.173 | 40.214 | 16.549 | 1.00 | 24.25 |
| ATOM | 4722 | O | GLU | B | 796 | −5.637 | 40.011 | 15.423 | 1.00 | 25.17 |
| ATOM | 4723 | N | LEU | B | 797 | −4.922 | 39.228 | 17.399 | 1.00 | 22.33 |
| ATOM | 4724 | CA | LEU | B | 797 | −5.201 | 37.847 | 17.021 | 1.00 | 21.19 |
| ATOM | 4725 | CB | LEU | B | 797 | −3.923 | 36.999 | 17.111 | 1.00 | 24.95 |
| ATOM | 4726 | CG | LEU | B | 797 | −2.757 | 37.369 | 16.184 | 1.00 | 24.74 |
| ATOM | 4727 | CD1 | LEU | B | 797 | −1.555 | 36.486 | 16.498 | 1.00 | 24.36 |
| ATOM | 4728 | CD2 | LEU | B | 797 | −3.173 | 37.209 | 14.720 | 1.00 | 25.36 |
| ATOM | 4729 | C | LEU | B | 797 | −6.295 | 37.239 | 17.889 | 1.00 | 22.61 |
| ATOM | 4730 | O | LEU | B | 797 | −6.717 | 36.108 | 17.657 | 1.00 | 21.44 |
| ATOM | 4731 | N | ASN | B | 798 | −6.756 | 38.002 | 18.877 | 1.00 | 22.45 |
| ATOM | 4732 | CA | ASN | B | 798 | −7.807 | 37.556 | 19.791 | 1.00 | 23.65 |
| ATOM | 4733 | CB | ASN | B | 798 | −9.165 | 37.569 | 19.089 | 1.00 | 32.89 |
| ATOM | 4734 | CG | ASN | B | 798 | −10.163 | 38.479 | 19.777 | 1.00 | 34.23 |
| ATOM | 4735 | OD1 | ASN | B | 798 | −10.392 | 38.372 | 20.982 | 1.00 | 32.44 |
| ATOM | 4736 | ND2 | ASN | B | 798 | −10.767 | 39.381 | 19.010 | 1.00 | 35.38 |
| ATOM | 4737 | C | ASN | B | 798 | −7.532 | 36.164 | 20.352 | 1.00 | 23.21 |
| ATOM | 4738 | O | ASN | B | 798 | −8.414 | 35.305 | 20.384 | 1.00 | 23.77 |
| ATOM | 4739 | N | ILE | B | 799 | −6.299 | 35.945 | 20.794 | 1.00 | 22.30 |
| ATOM | 4740 | CA | ILE | B | 799 | −5.914 | 34.655 | 21.359 | 1.00 | 24.01 |
| ATOM | 4741 | CB | ILE | B | 799 | −4.993 | 33.868 | 20.403 | 1.00 | 37.22 |
| ATOM | 4742 | CG2 | ILE | B | 799 | −5.661 | 33.695 | 19.047 | 1.00 | 37.98 |
| ATOM | 4743 | CG1 | ILE | B | 799 | −3.664 | 34.613 | 20.244 | 1.00 | 37.41 |
| ATOM | 4744 | CD1 | ILE | B | 799 | −2.556 | 33.780 | 19.633 | 1.00 | 37.91 |
| ATOM | 4745 | C | ILE | B | 799 | −5.146 | 34.867 | 22.658 | 1.00 | 23.86 |
| ATOM | 4746 | O | ILE | B | 799 | −4.542 | 35.922 | 22.867 | 1.00 | 22.69 |
| ATOM | 4747 | N | GLU | B | 800 | −5.178 | 33.870 | 23.533 | 1.00 | 27.63 |
| ATOM | 4748 | CA | GLU | B | 800 | −4.442 | 33.961 | 24.787 | 1.00 | 28.49 |
| ATOM | 4749 | CB | GLU | B | 800 | −4.912 | 32.882 | 25.766 | 1.00 | 54.96 |
| ATOM | 4750 | CG | GLU | B | 800 | −4.159 | 32.874 | 27.091 | 1.00 | 55.87 |
| ATOM | 4751 | CD | GLU | B | 800 | −4.612 | 31.758 | 28.018 | 1.00 | 56.25 |
| ATOM | 4752 | OE1 | GLU | B | 800 | −4.037 | 31.633 | 29.121 | 1.00 | 56.19 |
| ATOM | 4753 | OE2 | GLU | B | 800 | −5.541 | 31.008 | 27.645 | 1.00 | 56.33 |
| ATOM | 4754 | C | GLU | B | 800 | −2.995 | 33.711 | 24.385 | 1.00 | 28.05 |
| ATOM | 4755 | O | GLU | B | 800 | −2.712 | 32.791 | 23.616 | 1.00 | 28.04 |
| ATOM | 4756 | N | PRO | B | 801 | −2.060 | 34.539 | 24.866 | 1.00 | 26.29 |
| ATOM | 4757 | CD | PRO | B | 801 | −2.159 | 35.766 | 25.673 | 1.00 | 32.02 |
| ATOM | 4758 | CA | PRO | B | 801 | −0.675 | 34.280 | 24.465 | 1.00 | 25.59 |
| ATOM | 4759 | CB | PRO | B | 801 | 0.047 | 35.561 | 24.875 | 1.00 | 32.25 |
| ATOM | 4760 | CG | PRO | B | 801 | −0.719 | 35.993 | 26.085 | 1.00 | 32.69 |
| ATOM | 4761 | C | PRO | B | 801 | −0.115 | 33.044 | 25.155 | 1.00 | 25.64 |
| ATOM | 4762 | O | PRO | B | 801 | −0.688 | 32.552 | 26.127 | 1.00 | 26.27 |
| ATOM | 4763 | N | THR | B | 802 | 0.990 | 32.529 | 24.634 | 1.00 | 27.06 |
| ATOM | 4764 | CA | THR | B | 802 | 1.622 | 31.366 | 25.242 | 1.00 | 27.00 |
| ATOM | 4765 | CB | THR | B | 802 | 2.731 | 30.798 | 24.340 | 1.00 | 41.37 |
| ATOM | 4766 | OG1 | THR | B | 802 | 3.289 | 29.626 | 24.947 | 1.00 | 42.49 |
| ATOM | 4767 | CG2 | THR | B | 802 | 3.824 | 31.831 | 24.124 | 1.00 | 41.44 |
| ATOM | 4768 | C | THR | B | 802 | 2.220 | 31.839 | 26.562 | 1.00 | 26.88 |
| ATOM | 4769 | O | THR | B | 802 | 2.390 | 33.041 | 26.776 | 1.00 | 25.80 |
| ATOM | 4770 | N | ASP | B | 803 | 2.526 | 30.902 | 27.449 | 1.00 | 25.95 |
| ATOM | 4771 | CA | ASP | B | 803 | 3.096 | 31.248 | 28.744 | 1.00 | 25.37 |
| ATOM | 4772 | CB | ASP | B | 803 | 3.534 | 29.974 | 29.469 | 1.00 | 34.61 |
| ATOM | 4773 | CG | ASP | B | 803 | 2.374 | 29.260 | 30.141 | 1.00 | 34.77 |
| ATOM | 4774 | OD1 | ASP | B | 803 | 1.263 | 29.252 | 29.568 | 1.00 | 34.79 |
| ATOM | 4775 | OD2 | ASP | B | 803 | 2.581 | 28.699 | 31.238 | 1.00 | 35.35 |
| ATOM | 4776 | C | ASP | B | 803 | 4.260 | 32.240 | 28.673 | 1.00 | 24.64 |
| ATOM | 4777 | O | ASP | B | 803 | 4.283 | 33.226 | 29.409 | 1.00 | 25.72 |
| ATOM | 4778 | N | LEU | B | 804 | 5.217 | 31.999 | 27.785 | 1.00 | 27.10 |
| ATOM | 4779 | CA | LEU | B | 804 | 6.359 | 32.899 | 27.686 | 1.00 | 26.94 |
| ATOM | 4780 | CB | LEU | B | 804 | 7.423 | 32.300 | 26.757 | 1.00 | 52.26 |
| ATOM | 4781 | CG | LEU | B | 804 | 7.015 | 31.757 | 25.390 | 1.00 | 53.10 |
| ATOM | 4782 | CD1 | LEU | B | 804 | 7.196 | 32.830 | 24.331 | 1.00 | 53.31 |
| ATOM | 4783 | CD2 | LEU | B | 804 | 7.882 | 30.551 | 25.052 | 1.00 | 53.28 |
| ATOM | 4784 | C | LEU | B | 804 | 6.012 | 34.329 | 27.268 | 1.00 | 26.18 |
| ATOM | 4785 | O | LEU | B | 804 | 6.806 | 35.240 | 27.474 | 1.00 | 26.67 |
| ATOM | 4786 | N | MET | B | 805 | 4.823 | 34.537 | 26.705 | 1.00 | 24.63 |
| ATOM | 4787 | CA | MET | B | 805 | 4.423 | 35.880 | 26.285 | 1.00 | 24.58 |
| ATOM | 4788 | CB | MET | B | 805 | 4.151 | 35.902 | 24.780 | 1.00 | 30.72 |
| ATOM | 4789 | CG | MET | B | 805 | 5.333 | 35.419 | 23.963 | 1.00 | 31.20 |
| ATOM | 4790 | SD | MET | B | 805 | 5.137 | 35.642 | 22.209 | 1.00 | 31.94 |
| ATOM | 4791 | CE | MET | B | 805 | 6.846 | 35.924 | 21.734 | 1.00 | 31.24 |
| ATOM | 4792 | C | MET | B | 805 | 3.191 | 36.373 | 27.044 | 1.00 | 23.91 |
| ATOM | 4793 | O | MET | B | 805 | 2.552 | 37.352 | 26.647 | 1.00 | 23.70 |
| ATOM | 4794 | N | ASN | B | 806 | 2.884 | 35.699 | 28.147 | 1.00 | 22.71 |
| ATOM | 4795 | CA | ASN | B | 806 | 1.728 | 36.042 | 28.970 | 1.00 | 23.25 |
| ATOM | 4796 | CB | ASN | B | 806 | 0.917 | 34.772 | 29.255 | 1.00 | 28.25 |
| ATOM | 4797 | CG | ASN | B | 806 | −0.352 | 35.045 | 30.054 | 1.00 | 27.90 |
| ATOM | 4798 | OD1 | ASN | B | 806 | −0.642 | 36.181 | 30.420 | 1.00 | 28.09 |
| ATOM | 4799 | ND2 | ASN | B | 806 | −1.114 | 33.991 | 30.325 | 1.00 | 28.50 |
| ATOM | 4800 | C | ASN | B | 806 | 2.177 | 36.683 | 30.281 | 1.00 | 23.54 |
| ATOM | 4801 | O | ASN | B | 806 | 2.651 | 35.992 | 31.178 | 1.00 | 24.88 |
| ATOM | 4802 | N | ARG | B | 807 | 2.023 | 38.003 | 30.386 | 1.00 | 22.01 |
| ATOM | 4803 | CA | ARG | B | 807 | 2.417 | 38.748 | 31.585 | 1.00 | 22.16 |
| ATOM | 4804 | CB | ARG | B | 807 | 2.040 | 40.223 | 31.426 | 1.00 | 30.21 |
| ATOM | 4805 | CG | ARG | B | 807 | 2.729 | 41.167 | 32.399 | 1.00 | 30.83 |
| ATOM | 4806 | CD | ARG | B | 807 | 2.164 | 42.572 | 32.270 | 1.00 | 30.96 |
| ATOM | 4807 | NE | ARG | B | 807 | 2.118 | 43.013 | 30.879 | 1.00 | 31.52 |
| ATOM | 4808 | CZ | ARG | B | 807 | 3.161 | 43.482 | 30.205 | 1.00 | 31.30 |
| ATOM | 4809 | NH1 | ARG | B | 807 | 4.347 | 43.583 | 30.795 | 1.00 | 31.76 |
| ATOM | 4810 | NH2 | ARG | B | 807 | 3.019 | 43.843 | 28.935 | 1.00 | 30.41 |
| ATOM | 4811 | C | ARG | B | 807 | 1.753 | 38.186 | 32.844 | 1.00 | 23.50 |
| ATOM | 4812 | O | ARG | B | 807 | 2.324 | 38.241 | 33.933 | 1.00 | 22.48 |
| ATOM | 4813 | N | GLU | B | 808 | 0.549 | 37.645 | 32.694 | 1.00 | 27.30 |
| ATOM | 4814 | CA | GLU | B | 808 | −0.164 | 37.075 | 33.833 | 1.00 | 27.61 |
| ATOM | 4815 | CB | GLU | B | 808 | −1.585 | 36.680 | 33.421 | 1.00 | 44.18 |
| ATOM | 4816 | CG | GLU | B | 808 | −2.572 | 37.835 | 33.427 | 1.00 | 44.75 |
| ATOM | 4817 | CD | GLU | B | 808 | −3.887 | 37.494 | 32.750 | 1.00 | 45.10 |
| ATOM | 4818 | OE1 | GLU | B | 808 | −4.418 | 36.388 | 32.990 | 1.00 | 44.66 |
| ATOM | 4819 | OE2 | GLU | B | 808 | −4.390 | 38.342 | 31.982 | 1.00 | 44.84 |
| ATOM | 4820 | C | GLU | B | 808 | 0.559 | 35.865 | 34.416 | 1.00 | 28.07 |
| ATOM | 4821 | O | GLU | B | 808 | 0.426 | 35.572 | 35.604 | 1.00 | 27.52 |
| ATOM | 4822 | N | LYS | B | 809 | 1.328 | 35.171 | 33.582 | 1.00 | 30.73 |
| ATOM | 4823 | CA | LYS | B | 809 | 2.063 | 33.984 | 34.015 | 1.00 | 31.34 |
| ATOM | 4824 | CB | LYS | B | 809 | 1.888 | 32.856 | 32.988 | 1.00 | 44.52 |
| ATOM | 4825 | CG | LYS | B | 809 | 0.443 | 32.434 | 32.739 | 1.00 | 44.86 |
| ATOM | 4826 | CD | LYS | B | 809 | −0.095 | 31.554 | 33.853 | 1.00 | 45.24 |
| ATOM | 4827 | CE | LYS | B | 809 | 0.499 | 30.152 | 33.794 | 1.00 | 45.08 |
| ATOM | 4828 | NZ | LYS | B | 809 | 0.023 | 29.386 | 32.607 | 1.00 | 45.32 |
| ATOM | 4829 | C | LYS | B | 809 | 3.549 | 34.280 | 34.187 | 1.00 | 31.41 |
| ATOM | 4830 | O | LYS | B | 809 | 4.389 | 33.396 | 34.014 | 1.00 | 31.64 |
| ATOM | 4831 | N | LYS | B | 810 | 3.874 | 35.522 | 34.534 | 1.00 | 29.88 |
| ATOM | 4832 | CA | LYS | B | 810 | 5.266 | 35.924 | 34.714 | 1.00 | 29.67 |
| ATOM | 4833 | CB | LYS | B | 810 | 5.350 | 37.417 | 35.033 | 1.00 | 35.65 |
| ATOM | 4834 | CG | LYS | B | 810 | 4.772 | 37.787 | 36.384 | 1.00 | 35.72 |
| ATOM | 4835 | CD | LYS | B | 810 | 4.977 | 39.256 | 36.681 | 1.00 | 36.06 |
| ATOM | 4836 | CE | LYS | B | 810 | 4.445 | 39.617 | 38.058 | 1.00 | 36.21 |
| ATOM | 4837 | NZ | LYS | B | 810 | 4.806 | 41.010 | 38.416 | 1.00 | 36.50 |
| ATOM | 4838 | C | LYS | B | 810 | 5.981 | 35.135 | 35.815 | 1.00 | 29.55 |
| ATOM | 4839 | O | LYS | B | 810 | 7.204 | 35.220 | 35.954 | 1.00 | 29.80 |
| ATOM | 4840 | N | ASN | B | 811 | 5.221 | 34.371 | 36.595 | 1.00 | 28.77 |
| ATOM | 4841 | CA | ASN | B | 811 | 5.809 | 33.576 | 37.672 | 1.00 | 28.26 |
| ATOM | 4842 | CB | ASN | B | 811 | 4.716 | 33.113 | 38.643 | 1.00 | 33.98 |
| ATOM | 4843 | CG | ASN | B | 811 | 3.760 | 32.122 | 38.012 | 1.00 | 33.96 |
| ATOM | 4844 | OD1 | ASN | B | 811 | 4.059 | 30.935 | 37.904 | 1.00 | 34.26 |
| ATOM | 4845 | ND2 | ASN | B | 811 | 2.607 | 32.611 | 37.578 | 1.00 | 34.21 |
| ATOM | 4846 | C | ASN | B | 811 | 6.558 | 32.364 | 37.122 | 1.00 | 28.78 |
| ATOM | 4847 | O | ASN | B | 811 | 7.277 | 31.681 | 37.852 | 1.00 | 28.05 |
| ATOM | 4848 | N | LYS | B | 812 | 6.391 | 32.112 | 35.826 | 1.00 | 27.42 |
| ATOM | 4849 | CA | LYS | B | 812 | 7.034 | 30.983 | 35.161 | 1.00 | 27.06 |
| ATOM | 4850 | CB | LYS | B | 812 | 6.175 | 30.518 | 33.980 | 1.00 | 41.02 |
| ATOM | 4851 | CG | LYS | B | 812 | 5.384 | 29.246 | 34.224 | 1.00 | 41.61 |
| ATOM | 4852 | CD | LYS | B | 812 | 4.485 | 29.360 | 35.436 | 1.00 | 41.66 |
| ATOM | 4853 | CE | LYS | B | 812 | 3.735 | 28.062 | 35.681 | 1.00 | 41.87 |
| ATOM | 4854 | NZ | LYS | B | 812 | 4.665 | 26.921 | 35.913 | 1.00 | 41.60 |
| ATOM | 4855 | C | LYS | B | 812 | 8.436 | 31.306 | 34.655 | 1.00 | 25.94 |
| ATOM | 4856 | O | LYS | B | 812 | 9.169 | 30.409 | 34.248 | 1.00 | 27.20 |
| ATOM | 4857 | N | ILE | B | 813 | 8.814 | 32.579 | 34.693 | 1.00 | 24.47 |
| ATOM | 4858 | CA | ILE | B | 813 | 10.126 | 32.993 | 34.203 | 1.00 | 24.87 |
| ATOM | 4859 | CB | ILE | B | 813 | 10.353 | 34.509 | 34.452 | 1.00 | 27.03 |
| ATOM | 4860 | CG2 | ILE | B | 813 | 11.812 | 34.887 | 34.190 | 1.00 | 26.32 |
| ATOM | 4861 | CG1 | ILE | B | 813 | 9.420 | 35.312 | 33.537 | 1.00 | 27.43 |
| ATOM | 4862 | CD1 | ILE | B | 813 | 9.422 | 36.809 | 33.792 | 1.00 | 27.47 |
| ATOM | 4863 | C | ILE | B | 813 | 11.321 | 32.198 | 34.725 | 1.00 | 24.94 |
| ATOM | 4864 | O | ILE | B | 813 | 12.153 | 31.747 | 33.940 | 1.00 | 24.37 |
| ATOM | 4865 | N | PRO | B | 814 | 11.433 | 32.015 | 36.051 | 1.00 | 23.33 |
| ATOM | 4866 | CD | PRO | B | 814 | 10.649 | 32.549 | 37.180 | 1.00 | 25.56 |
| ATOM | 4867 | CA | PRO | B | 814 | 12.588 | 31.251 | 36.536 | 1.00 | 24.96 |
| ATOM | 4868 | CB | PRO | B | 814 | 12.336 | 31.171 | 38.041 | 1.00 | 25.58 |
| ATOM | 4869 | CG | PRO | B | 814 | 11.650 | 32.478 | 38.317 | 1.00 | 25.37 |
| ATOM | 4870 | C | PRO | B | 814 | 12.708 | 29.870 | 35.892 | 1.00 | 24.30 |
| ATOM | 4871 | O | PRO | B | 814 | 13.752 | 29.519 | 35.346 | 1.00 | 26.00 |
| ATOM | 4872 | N | SER | B | 815 | 11.629 | 29.101 | 35.942 | 1.00 | 27.38 |
| ATOM | 4873 | CA | SER | B | 815 | 11.631 | 27.759 | 35.372 | 1.00 | 28.28 |
| ATOM | 4874 | CB | SER | B | 815 | 10.320 | 27.048 | 35.705 | 1.00 | 30.12 |
| ATOM | 4875 | OG | SER | B | 815 | 9.214 | 27.762 | 35.193 | 1.00 | 30.40 |
| ATOM | 4876 | C | SER | B | 815 | 11.852 | 27.767 | 33.860 | 1.00 | 28.08 |
| ATOM | 4877 | O | SER | B | 815 | 12.426 | 26.831 | 33.307 | 1.00 | 28.51 |
| ATOM | 4878 | N | MET | B | 816 | 11.405 | 28.822 | 33.190 | 1.00 | 26.53 |
| ATOM | 4879 | CA | MET | B | 816 | 11.581 | 28.899 | 31.743 | 1.00 | 25.98 |
| ATOM | 4880 | CB | MET | B | 816 | 10.633 | 29.940 | 31.147 | 1.00 | 38.18 |
| ATOM | 4881 | CG | MET | B | 816 | 9.173 | 29.635 | 31.422 | 1.00 | 38.60 |
| ATOM | 4882 | SD | MET | B | 816 | 8.038 | 30.687 | 30.502 | 1.00 | 40.03 |
| ATOM | 4883 | CE | MET | B | 816 | 8.543 | 32.319 | 31.070 | 1.00 | 38.78 |
| ATOM | 4884 | C | MET | B | 816 | 13.021 | 29.224 | 31.384 | 1.00 | 25.44 |
| ATOM | 4885 | O | MET | B | 816 | 13.540 | 28.736 | 30.383 | 1.00 | 25.22 |
| ATOM | 4886 | N | GLN | B | 817 | 13.666 | 30.047 | 32.207 | 1.00 | 24.56 |
| ATOM | 4887 | CA | GLN | B | 817 | 15.057 | 30.413 | 31.984 | 1.00 | 24.03 |
| ATOM | 4888 | CB | GLN | B | 817 | 15.503 | 31.465 | 33.000 | 1.00 | 23.33 |
| ATOM | 4889 | CG | GLN | B | 817 | 15.074 | 32.885 | 32.672 | 1.00 | 23.25 |
| ATOM | 4890 | CD | GLN | B | 817 | 15.603 | 33.349 | 31.331 | 1.00 | 24.23 |
| ATOM | 4891 | OE1 | GLN | B | 817 | 15.073 | 32.980 | 30.285 | 1.00 | 23.44 |
| ATOM | 4892 | NE2 | GLN | B | 817 | 16.666 | 34.146 | 31.354 | 1.00 | 23.50 |
| ATOM | 4893 | C | GLN | B | 817 | 15.923 | 29.166 | 32.122 | 1.00 | 24.21 |
| ATOM | 4894 | O | GLN | B | 817 | 16.862 | 28.961 | 31.350 | 1.00 | 25.39 |
| ATOM | 4895 | N | VAL | B | 818 | 15.609 | 28.342 | 33.116 | 1.00 | 22.52 |
| ATOM | 4896 | CA | VAL | B | 818 | 16.350 | 27.103 | 33.331 | 1.00 | 24.31 |
| ATOM | 4897 | CB | VAL | B | 818 | 15.820 | 26.335 | 34.562 | 1.00 | 26.47 |
| ATOM | 4898 | CG1 | VAL | B | 818 | 16.541 | 24.993 | 34.688 | 1.00 | 26.36 |
| ATOM | 4899 | CG2 | VAL | B | 818 | 16.037 | 27.164 | 35.815 | 1.00 | 26.36 |
| ATOM | 4900 | C | VAL | B | 818 | 16.182 | 26.236 | 32.085 | 1.00 | 23.82 |
| ATOM | 4901 | O | VAL | B | 818 | 17.148 | 25.659 | 31.588 | 1.00 | 23.44 |
| ATOM | 4902 | N | GLY | B | 819 | 14.953 | 26.172 | 31.577 | 1.00 | 26.53 |
| ATOM | 4903 | CA | GLY | B | 819 | 14.669 | 25.386 | 30.385 | 1.00 | 25.97 |
| ATOM | 4904 | C | GLY | B | 819 | 15.410 | 25.903 | 29.164 | 1.00 | 25.98 |
| ATOM | 4905 | O | GLY | B | 819 | 15.928 | 25.128 | 28.365 | 1.00 | 25.51 |
| ATOM | 4906 | N | PHE | B | 820 | 15.455 | 27.223 | 29.021 | 1.00 | 22.89 |
| ATOM | 4907 | CA | PHE | B | 820 | 16.149 | 27.869 | 27.906 | 1.00 | 22.59 |
| ATOM | 4908 | CB | PHE | B | 820 | 15.941 | 29.389 | 27.997 | 1.00 | 24.78 |
| ATOM | 4909 | CG | PHE | B | 820 | 16.821 | 30.195 | 27.078 | 1.00 | 26.25 |
| ATOM | 4910 | CD1 | PHE | B | 820 | 16.716 | 30.071 | 25.696 | 1.00 | 25.90 |
| ATOM | 4911 | CD2 | PHE | B | 820 | 17.742 | 31.104 | 27.601 | 1.00 | 24.43 |
| ATOM | 4912 | CE1 | PHE | B | 820 | 17.513 | 30.841 | 24.845 | 1.00 | 25.64 |
| ATOM | 4913 | CE2 | PHE | B | 820 | 18.545 | 31.877 | 26.762 | 1.00 | 25.89 |
| ATOM | 4914 | CZ | PHE | B | 820 | 18.432 | 31.747 | 25.381 | 1.00 | 25.35 |
| ATOM | 4915 | C | PHE | B | 820 | 17.641 | 27.535 | 27.953 | 1.00 | 22.74 |
| ATOM | 4916 | O | PHE | B | 820 | 18.256 | 27.231 | 26.932 | 1.00 | 21.86 |
| ATOM | 4917 | N | ILE | B | 821 | 18.222 | 27.595 | 29.143 | 1.00 | 24.60 |
| ATOM | 4918 | CA | ILE | B | 821 | 19.640 | 27.303 | 29.295 | 1.00 | 26.01 |
| ATOM | 4919 | CB | ILE | B | 821 | 20.099 | 27.593 | 30.739 | 1.00 | 23.25 |
| ATOM | 4920 | CG2 | ILE | B | 821 | 21.480 | 27.001 | 30.984 | 1.00 | 23.21 |
| ATOM | 4921 | CG1 | ILE | B | 821 | 20.096 | 29.105 | 30.980 | 1.00 | 22.99 |
| ATOM | 4922 | CD1 | ILE | B | 821 | 20.409 | 29.506 | 32.410 | 1.00 | 23.26 |
| ATOM | 4923 | C | ILE | B | 821 | 19.944 | 25.848 | 28.929 | 1.00 | 26.27 |
| ATOM | 4924 | O | ILE | B | 821 | 20.893 | 25.565 | 28.187 | 1.00 | 26.66 |
| ATOM | 4925 | N | ASP | B | 822 | 19.129 | 24.931 | 29.436 | 1.00 | 29.94 |
| ATOM | 4926 | CA | ASP | B | 822 | 19.332 | 23.511 | 29.168 | 1.00 | 30.98 |
| ATOM | 4927 | CB | ASP | B | 822 | 18.391 | 22.657 | 30.023 | 1.00 | 31.31 |
| ATOM | 4928 | CG | ASP | B | 822 | 18.798 | 22.617 | 31.478 | 1.00 | 31.53 |
| ATOM | 4929 | OD1 | ASP | B | 822 | 19.984 | 22.882 | 31.771 | 1.00 | 31.53 |
| ATOM | 4930 | OD2 | ASP | B | 822 | 17.939 | 22.301 | 32.330 | 1.00 | 31.44 |
| ATOM | 4931 | C | ASP | B | 822 | 19.147 | 23.103 | 27.715 | 1.00 | 30.90 |
| ATOM | 4932 | O | ASP | B | 822 | 19.963 | 22.368 | 27.162 | 1.00 | 31.36 |
| ATOM | 4933 | N | ALA | B | 823 | 18.070 | 23.577 | 27.102 | 1.00 | 28.23 |
| ATOM | 4934 | CA | ALA | B | 823 | 17.757 | 23.217 | 25.724 | 1.00 | 27.21 |
| ATOM | 4935 | CB | ALA | B | 823 | 16.268 | 23.438 | 25.470 | 1.00 | 28.57 |
| ATOM | 4936 | C | ALA | B | 823 | 18.563 | 23.923 | 24.642 | 1.00 | 27.07 |
| ATOM | 4937 | O | ALA | B | 823 | 18.861 | 23.330 | 23.607 | 1.00 | 27.74 |
| ATOM | 4938 | N | ILE | B | 824 | 18.926 | 25.178 | 24.878 | 1.00 | 23.52 |
| ATOM | 4939 | CA | ILE | B | 824 | 19.639 | 25.950 | 23.869 | 1.00 | 23.73 |
| ATOM | 4940 | CB | ILE | B | 824 | 18.802 | 27.205 | 23.482 | 1.00 | 34.66 |
| ATOM | 4941 | CG2 | ILE | B | 824 | 19.646 | 28.198 | 22.686 | 1.00 | 35.04 |
| ATOM | 4942 | CG1 | ILE | B | 824 | 17.571 | 26.780 | 22.682 | 1.00 | 35.26 |
| ATOM | 4943 | CD1 | ILE | B | 824 | 17.912 | 26.136 | 21.354 | 1.00 | 35.75 |
| ATOM | 4944 | C | ILE | B | 824 | 21.052 | 26.416 | 24.195 | 1.00 | 23.62 |
| ATOM | 4945 | O | ILE | B | 824 | 21.963 | 26.256 | 23.387 | 1.00 | 24.76 |
| ATOM | 4946 | N | CYS | B | 825 | 21.242 | 26.982 | 25.381 | 1.00 | 25.35 |
| ATOM | 4947 | CA | CYS | B | 825 | 22.537 | 27.545 | 25.750 | 1.00 | 25.12 |
| ATOM | 4948 | CB | CYS | B | 825 | 22.322 | 28.602 | 26.827 | 1.00 | 22.81 |
| ATOM | 4949 | SG | CYS | B | 825 | 21.059 | 29.783 | 26.356 | 1.00 | 22.83 |
| ATOM | 4950 | C | CYS | B | 825 | 23.700 | 26.653 | 26.169 | 1.00 | 25.45 |
| ATOM | 4951 | O | CYS | B | 825 | 24.796 | 26.778 | 25.627 | 1.00 | 26.37 |
| ATOM | 4952 | N | LEU | B | 826 | 23.477 | 25.783 | 27.145 | 1.00 | 25.06 |
| ATOM | 4953 | CA | LEU | B | 826 | 24.541 | 24.921 | 27.651 | 1.00 | 26.86 |
| ATOM | 4954 | CB | LEU | B | 826 | 23.954 | 23.898 | 28.626 | 1.00 | 41.13 |
| ATOM | 4955 | CG | LEU | B | 826 | 24.523 | 23.958 | 30.048 | 1.00 | 41.95 |
| ATOM | 4956 | CD1 | LEU | B | 826 | 24.537 | 25.392 | 30.550 | 1.00 | 41.82 |
| ATOM | 4957 | CD2 | LEU | B | 826 | 23.695 | 23.076 | 30.967 | 1.00 | 41.66 |
| ATOM | 4958 | C | LEU | B | 826 | 25.397 | 24.208 | 26.606 | 1.00 | 25.29 |
| ATOM | 4959 | O | LEU | B | 826 | 26.623 | 24.327 | 26.619 | 1.00 | 27.23 |
| ATOM | 4960 | N | GLN | B | 827 | 24.764 | 23.467 | 25.703 | 1.00 | 27.86 |
| ATOM | 4961 | CA | GLN | B | 827 | 25.512 | 22.737 | 24.679 | 1.00 | 27.22 |
| ATOM | 4962 | CB | GLN | B | 827 | 24.560 | 21.935 | 23.796 | 1.00 | 43.35 |
| ATOM | 4963 | CG | GLN | B | 827 | 23.663 | 20.981 | 24.546 | 1.00 | 43.82 |
| ATOM | 4964 | CD | GLN | B | 827 | 22.683 | 20.289 | 23.626 | 1.00 | 44.18 |
| ATOM | 4965 | OE1 | GLN | B | 827 | 23.077 | 19.509 | 22.759 | 1.00 | 44.12 |
| ATOM | 4966 | NE2 | GLN | B | 827 | 21.396 | 20.582 | 23.800 | 1.00 | 44.14 |
| ATOM | 4967 | C | GLN | B | 827 | 26.346 | 23.652 | 23.791 | 1.00 | 27.96 |
| ATOM | 4968 | O | GLN | B | 827 | 27.469 | 23.310 | 23.417 | 1.00 | 27.98 |
| ATOM | 4969 | N | LEU | B | 828 | 25.789 | 24.807 | 23.439 | 1.00 | 23.58 |
| ATOM | 4970 | CA | LEU | B | 828 | 26.491 | 25.753 | 22.585 | 1.00 | 23.82 |
| ATOM | 4971 | CB | LEU | B | 828 | 25.574 | 26.922 | 22.212 | 1.00 | 24.08 |
| ATOM | 4972 | CG | LEU | B | 828 | 26.222 | 28.012 | 21.353 | 1.00 | 24.69 |
| ATOM | 4973 | CD1 | LEU | B | 828 | 26.835 | 27.406 | 20.087 | 1.00 | 24.52 |
| ATOM | 4974 | CD2 | LEU | B | 828 | 25.180 | 29.058 | 21.001 | 1.00 | 24.55 |
| ATOM | 4975 | C | LEU | B | 828 | 27.768 | 26.289 | 23.218 | 1.00 | 23.20 |
| ATOM | 4976 | O | LEU | B | 828 | 28.824 | 26.307 | 22.586 | 1.00 | 23.94 |
| ATOM | 4977 | N | TYR | B | 829 | 27.681 | 26.736 | 24.464 | 1.00 | 24.32 |
| ATOM | 4978 | CA | TYR | B | 829 | 28.866 | 27.266 | 25.122 | 1.00 | 24.21 |
| ATOM | 4979 | CB | TYR | B | 829 | 28.461 | 28.040 | 26.378 | 1.00 | 23.28 |
| ATOM | 4980 | CG | TYR | B | 829 | 27.724 | 29.315 | 26.015 | 1.00 | 21.35 |
| ATOM | 4981 | CD1 | TYR | B | 829 | 28.358 | 30.318 | 25.280 | 1.00 | 22.17 |
| ATOM | 4982 | CE1 | TYR | B | 829 | 27.673 | 31.462 | 24.866 | 1.00 | 21.77 |
| ATOM | 4983 | CD2 | TYR | B | 829 | 26.383 | 29.488 | 26.337 | 1.00 | 23.05 |
| ATOM | 4984 | CE2 | TYR | B | 829 | 25.686 | 30.630 | 25.928 | 1.00 | 21.86 |
| ATOM | 4985 | CZ | TYR | B | 829 | 26.338 | 31.609 | 25.192 | 1.00 | 22.76 |
| ATOM | 4986 | OH | TYR | B | 829 | 25.662 | 32.733 | 24.759 | 1.00 | 20.79 |
| ATOM | 4987 | C | TYR | B | 829 | 29.871 | 26.163 | 25.428 | 1.00 | 25.08 |
| ATOM | 4988 | O | TYR | B | 829 | 31.076 | 26.408 | 25.465 | 1.00 | 24.92 |
| ATOM | 4989 | N | GLU | B | 830 | 29.380 | 24.944 | 25.628 | 1.00 | 25.46 |
| ATOM | 4990 | CA | GLU | B | 830 | 30.278 | 23.826 | 25.892 | 1.00 | 27.34 |
| ATOM | 4991 | CB | GLU | B | 830 | 29.482 | 22.572 | 26.268 | 1.00 | 35.78 |
| ATOM | 4992 | CG | GLU | B | 830 | 28.822 | 22.637 | 27.635 | 1.00 | 36.70 |
| ATOM | 4993 | CD | GLU | B | 830 | 27.981 | 21.409 | 27.938 | 1.00 | 36.51 |
| ATOM | 4994 | OE1 | GLU | B | 830 | 27.433 | 21.318 | 29.056 | 1.00 | 36.91 |
| ATOM | 4995 | OE2 | GLU | B | 830 | 27.863 | 20.533 | 27.053 | 1.00 | 37.38 |
| ATOM | 4996 | C | GLU | B | 830 | 31.087 | 23.563 | 24.624 | 1.00 | 26.06 |
| ATOM | 4997 | O | GLU | B | 830 | 32.305 | 23.377 | 24.679 | 1.00 | 26.54 |
| ATOM | 4998 | N | ALA | B | 831 | 30.398 | 23.566 | 23.486 | 1.00 | 25.64 |
| ATOM | 4999 | CA | ALA | B | 831 | 31.026 | 23.332 | 22.189 | 1.00 | 23.78 |
| ATOM | 5000 | CB | ALA | B | 831 | 29.952 | 23.253 | 21.093 | 1.00 | 30.54 |
| ATOM | 5001 | C | ALA | B | 831 | 32.031 | 24.427 | 21.853 | 1.00 | 24.89 |
| ATOM | 5002 | O | ALA | B | 831 | 33.110 | 24.151 | 21.328 | 1.00 | 24.70 |
| ATOM | 5003 | N | LEU | B | 832 | 31.679 | 25.671 | 22.158 | 1.00 | 22.52 |
| ATOM | 5004 | CA | LEU | B | 832 | 32.578 | 26.778 | 21.872 | 1.00 | 23.49 |
| ATOM | 5005 | CB | LEU | B | 832 | 31.905 | 28.116 | 22.198 | 1.00 | 21.26 |
| ATOM | 5006 | CG | LEU | B | 832 | 32.754 | 29.333 | 21.824 | 1.00 | 22.68 |
| ATOM | 5007 | CD1 | LEU | B | 832 | 33.199 | 29.213 | 20.369 | 1.00 | 21.78 |
| ATOM | 5008 | CD2 | LEU | B | 832 | 31.963 | 30.613 | 22.042 | 1.00 | 22.23 |
| ATOM | 5009 | C | LEU | B | 832 | 33.854 | 26.627 | 22.694 | 1.00 | 22.63 |
| ATOM | 5010 | O | LEU | B | 832 | 34.955 | 26.916 | 22.220 | 1.00 | 23.62 |
| ATOM | 5011 | N | THR | B | 833 | 33.697 | 26.165 | 23.928 | 1.00 | 26.15 |
| ATOM | 5012 | CA | THR | B | 833 | 34.828 | 25.973 | 24.822 | 1.00 | 27.69 |
| ATOM | 5013 | CB | THR | B | 833 | 34.350 | 25.621 | 26.247 | 1.00 | 25.51 |
| ATOM | 5014 | OG1 | THR | B | 833 | 33.549 | 26.695 | 26.759 | 1.00 | 27.15 |
| ATOM | 5015 | CG2 | THR | B | 833 | 35.539 | 25.401 | 27.181 | 1.00 | 26.42 |
| ATOM | 5016 | C | THR | B | 833 | 35.733 | 24.862 | 24.293 | 1.00 | 27.86 |
| ATOM | 5017 | O | THR | B | 833 | 36.949 | 24.876 | 24.506 | 1.00 | 27.78 |
| ATOM | 5018 | N | HIS | B | 834 | 35.132 | 23.904 | 23.600 | 1.00 | 29.42 |
| ATOM | 5019 | CA | HIS | B | 834 | 35.885 | 22.795 | 23.026 | 1.00 | 30.11 |
| ATOM | 5020 | CB | HIS | B | 834 | 34.920 | 21.755 | 22.449 | 1.00 | 46.85 |
| ATOM | 5021 | CG | HIS | B | 834 | 35.571 | 20.457 | 22.084 | 1.00 | 47.63 |
| ATOM | 5022 | CD2 | HIS | B | 834 | 36.207 | 20.064 | 20.955 | 1.00 | 47.79 |
| ATOM | 5023 | ND1 | HIS | B | 834 | 35.621 | 19.382 | 22.945 | 1.00 | 47.75 |
| ATOM | 5024 | CE1 | HIS | B | 834 | 36.257 | 18.381 | 22.361 | 1.00 | 47.72 |
| ATOM | 5025 | NE2 | HIS | B | 834 | 36.623 | 18.769 | 21.153 | 1.00 | 47.50 |
| ATOM | 5026 | C | HIS | B | 834 | 36.773 | 23.351 | 21.912 | 1.00 | 30.08 |
| ATOM | 5027 | O | HIS | B | 834 | 37.939 | 22.972 | 21.773 | 1.00 | 30.93 |
| ATOM | 5028 | N | VAL | B | 835 | 36.209 | 24.259 | 21.119 | 1.00 | 25.19 |
| ATOM | 5029 | CA | VAL | B | 835 | 36.935 | 24.866 | 20.015 | 1.00 | 25.37 |
| ATOM | 5030 | CB | VAL | B | 835 | 35.986 | 25.730 | 19.144 | 1.00 | 27.06 |
| ATOM | 5031 | CG1 | VAL | B | 835 | 36.764 | 26.460 | 18.071 | 1.00 | 27.88 |
| ATOM | 5032 | CG2 | VAL | B | 835 | 34.932 | 24.842 | 18.505 | 1.00 | 27.32 |
| ATOM | 5033 | C | VAL | B | 835 | 38.107 | 25.704 | 20.515 | 1.00 | 25.36 |
| ATOM | 5034 | O | VAL | B | 835 | 39.204 | 25.626 | 19.971 | 1.00 | 25.68 |
| ATOM | 5035 | N | SER | B | 836 | 37.874 | 26.504 | 21.552 | 1.00 | 28.63 |
| ATOM | 5036 | CA | SER | B | 836 | 38.926 | 27.340 | 22.130 | 1.00 | 29.30 |
| ATOM | 5037 | CB | SER | B | 836 | 38.924 | 28.734 | 21.489 | 1.00 | 32.06 |
| ATOM | 5038 | OG | SER | B | 836 | 39.982 | 29.529 | 21.996 | 1.00 | 32.73 |
| ATOM | 5039 | C | SER | B | 836 | 38.674 | 27.454 | 23.629 | 1.00 | 29.51 |
| ATOM | 5040 | O | SER | B | 836 | 37.718 | 28.096 | 24.058 | 1.00 | 28.84 |
| ATOM | 5041 | N | GLU | B | 837 | 39.530 | 26.825 | 24.426 | 1.00 | 26.81 |
| ATOM | 5042 | CA | GLU | B | 837 | 39.359 | 26.846 | 25.874 | 1.00 | 27.10 |
| ATOM | 5043 | CB | GLU | B | 837 | 40.430 | 25.972 | 26.536 | 1.00 | 50.60 |
| ATOM | 5044 | CG | GLU | B | 837 | 41.843 | 26.245 | 26.062 | 1.00 | 51.48 |
| ATOM | 5045 | CD | GLU | B | 837 | 42.811 | 25.145 | 26.460 | 1.00 | 51.89 |
| ATOM | 5046 | OE1 | GLU | B | 837 | 42.985 | 24.912 | 27.677 | 1.00 | 51.52 |
| ATOM | 5047 | OE2 | GLU | B | 837 | 43.394 | 24.512 | 25.552 | 1.00 | 51.73 |
| ATOM | 5048 | C | GLU | B | 837 | 39.348 | 28.242 | 26.494 | 1.00 | 26.43 |
| ATOM | 5049 | O | GLU | B | 837 | 38.902 | 28.413 | 27.628 | 1.00 | 27.28 |
| ATOM | 5050 | N | ASP | B | 838 | 39.819 | 29.238 | 25.749 | 1.00 | 24.88 |
| ATOM | 5051 | CA | ASP | B | 838 | 39.846 | 30.604 | 26.254 | 1.00 | 25.77 |
| ATOM | 5052 | CB | ASP | B | 838 | 40.811 | 31.457 | 25.428 | 1.00 | 38.63 |
| ATOM | 5053 | CG | ASP | B | 838 | 42.256 | 31.011 | 25.582 | 1.00 | 39.53 |
| ATOM | 5054 | OD1 | ASP | B | 838 | 42.765 | 31.036 | 26.724 | 1.00 | 39.53 |
| ATOM | 5055 | OD2 | ASP | B | 838 | 42.877 | 30.633 | 24.568 | 1.00 | 39.29 |
| ATOM | 5056 | C | ASP | B | 838 | 38.449 | 31.226 | 26.252 | 1.00 | 25.03 |
| ATOM | 5057 | O | ASP | B | 838 | 38.277 | 32.360 | 26.699 | 1.00 | 25.25 |
| ATOM | 5058 | N | CYS | B | 839 | 37.464 | 30.483 | 25.748 | 1.00 | 29.32 |
| ATOM | 5059 | CA | CYS | B | 839 | 36.073 | 30.949 | 25.721 | 1.00 | 28.67 |
| ATOM | 5060 | CB | CYS | B | 839 | 35.383 | 30.561 | 24.400 | 1.00 | 25.46 |
| ATOM | 5061 | SG | CYS | B | 839 | 35.809 | 31.580 | 22.967 | 1.00 | 23.35 |
| ATOM | 5062 | C | CYS | B | 839 | 35.299 | 30.342 | 26.895 | 1.00 | 29.69 |
| ATOM | 5063 | O | CYS | B | 839 | 34.075 | 30.468 | 26.984 | 1.00 | 29.52 |
| ATOM | 5064 | N | PHE | B | 840 | 36.023 | 29.686 | 27.798 | 1.00 | 26.11 |
| ATOM | 5065 | CA | PHE | B | 840 | 35.405 | 29.055 | 28.960 | 1.00 | 25.54 |
| ATOM | 5066 | CB | PHE | B | 840 | 36.467 | 28.387 | 29.834 | 1.00 | 29.48 |
| ATOM | 5067 | CG | PHE | B | 840 | 35.916 | 27.797 | 31.101 | 1.00 | 29.46 |
| ATOM | 5068 | CD1 | PHE | B | 840 | 35.084 | 26.679 | 31.060 | 1.00 | 29.72 |
| ATOM | 5069 | CD2 | PHE | B | 840 | 36.204 | 28.373 | 32.333 | 1.00 | 29.95 |
| ATOM | 5070 | CE1 | PHE | B | 840 | 34.546 | 26.143 | 32.231 | 1.00 | 30.22 |
| ATOM | 5071 | CE2 | PHE | B | 840 | 35.671 | 27.845 | 33.512 | 1.00 | 29.42 |
| ATOM | 5072 | CZ | PHE | B | 840 | 34.840 | 26.729 | 33.460 | 1.00 | 29.68 |
| ATOM | 5073 | C | PHE | B | 840 | 34.567 | 29.996 | 29.831 | 1.00 | 26.06 |
| ATOM | 5074 | O | PHE | B | 840 | 33.531 | 29.593 | 30.354 | 1.00 | 26.34 |
| ATOM | 5075 | N | PRO | B | 841 | 35.007 | 31.253 | 30.013 | 1.00 | 25.13 |
| ATOM | 5076 | CD | PRO | B | 841 | 36.255 | 31.904 | 29.575 | 1.00 | 24.36 |
| ATOM | 5077 | CA | PRO | B | 841 | 34.210 | 32.164 | 30.846 | 1.00 | 24.99 |
| ATOM | 5078 | CB | PRO | B | 841 | 34.927 | 33.502 | 30.673 | 1.00 | 24.78 |
| ATOM | 5079 | CG | PRO | B | 841 | 36.368 | 33.073 | 30.535 | 1.00 | 24.46 |
| ATOM | 5080 | C | PRO | B | 841 | 32.729 | 32.229 | 30.446 | 1.00 | 24.81 |
| ATOM | 5081 | O | PRO | B | 841 | 31.865 | 32.393 | 31.300 | 1.00 | 25.33 |
| ATOM | 5082 | N | LEU | B | 842 | 32.435 | 32.096 | 29.155 | 1.00 | 24.08 |
| ATOM | 5083 | CA | LEU | B | 842 | 31.044 | 32.143 | 28.707 | 1.00 | 22.49 |
| ATOM | 5084 | CB | LEU | B | 842 | 30.972 | 32.171 | 27.177 | 1.00 | 19.33 |
| ATOM | 5085 | CG | LEU | B | 842 | 31.614 | 33.377 | 26.488 | 1.00 | 20.20 |
| ATOM | 5086 | CD1 | LEU | B | 842 | 31.712 | 33.118 | 24.985 | 1.00 | 19.25 |
| ATOM | 5087 | CD2 | LEU | B | 842 | 30.794 | 34.632 | 26.783 | 1.00 | 20.71 |
| ATOM | 5088 | C | LEU | B | 842 | 30.260 | 30.944 | 29.242 | 1.00 | 22.87 |
| ATOM | 5089 | O | LEU | B | 842 | 29.143 | 31.088 | 29.732 | 1.00 | 23.13 |
| ATOM | 5090 | N | LEU | B | 843 | 30.855 | 29.760 | 29.158 | 1.00 | 22.66 |
| ATOM | 5091 | CA | LEU | B | 843 | 30.195 | 28.553 | 29.640 | 1.00 | 22.53 |
| ATOM | 5092 | CB | LEU | B | 843 | 31.032 | 27.317 | 29.298 | 1.00 | 33.57 |
| ATOM | 5093 | CG | LEU | B | 843 | 30.366 | 25.935 | 29.275 | 1.00 | 34.94 |
| ATOM | 5094 | CD1 | LEU | B | 843 | 31.458 | 24.882 | 29.157 | 1.00 | 34.20 |
| ATOM | 5095 | CD2 | LEU | B | 843 | 29.540 | 25.691 | 30.515 | 1.00 | 34.62 |
| ATOM | 5096 | C | LEU | B | 843 | 30.024 | 28.635 | 31.153 | 1.00 | 22.75 |
| ATOM | 5097 | O | LEU | B | 843 | 28.976 | 28.280 | 31.690 | 1.00 | 22.24 |
| ATOM | 5098 | N | ASP | B | 844 | 31.068 | 29.101 | 31.831 | 1.00 | 25.80 |
| ATOM | 5099 | CA | ASP | B | 844 | 31.061 | 29.228 | 33.283 | 1.00 | 26.51 |
| ATOM | 5100 | CB | ASP | B | 844 | 32.425 | 29.733 | 33.761 | 1.00 | 37.17 |
| ATOM | 5101 | CG | ASP | B | 844 | 32.584 | 29.653 | 35.267 | 1.00 | 37.71 |
| ATOM | 5102 | OD1 | ASP | B | 844 | 32.277 | 28.589 | 35.844 | 1.00 | 37.75 |
| ATOM | 5103 | OD2 | ASP | B | 844 | 33.028 | 30.654 | 35.871 | 1.00 | 38.29 |
| ATOM | 5104 | C | ASP | B | 844 | 29.953 | 30.171 | 33.750 | 1.00 | 26.88 |
| ATOM | 5105 | O | ASP | B | 844 | 29.215 | 29.855 | 34.683 | 1.00 | 25.76 |
| ATOM | 5106 | N | GLY | B | 845 | 29.840 | 31.323 | 33.093 | 1.00 | 27.19 |
| ATOM | 5107 | CA | GLY | B | 845 | 28.811 | 32.281 | 33.457 | 1.00 | 27.20 |
| ATOM | 5108 | C | GLY | B | 845 | 27.418 | 31.719 | 33.239 | 1.00 | 27.51 |
| ATOM | 5109 | O | GLY | B | 845 | 26.511 | 31.944 | 34.043 | 1.00 | 26.76 |
| ATOM | 5110 | N | CYS | B | 846 | 27.246 | 30.982 | 32.147 | 1.00 | 23.86 |
| ATOM | 5111 | CA | CYS | B | 846 | 25.956 | 30.381 | 31.838 | 1.00 | 24.52 |
| ATOM | 5112 | CB | CYS | B | 846 | 26.026 | 29.636 | 30.505 | 1.00 | 23.78 |
| ATOM | 5113 | SG | CYS | B | 846 | 24.460 | 28.924 | 29.962 | 1.00 | 22.31 |
| ATOM | 5114 | C | CYS | B | 846 | 25.538 | 29.414 | 32.938 | 1.00 | 24.60 |
| ATOM | 5115 | O | CYS | B | 846 | 24.388 | 29.414 | 33.373 | 1.00 | 24.31 |
| ATOM | 5116 | N | ARG | B | 847 | 26.477 | 28.589 | 33.394 | 1.00 | 26.53 |
| ATOM | 5117 | CA | ARG | B | 847 | 26.174 | 27.621 | 34.438 | 1.00 | 26.50 |
| ATOM | 5118 | CB | ARG | B | 847 | 27.317 | 26.605 | 34.556 | 1.00 | 46.23 |
| ATOM | 5119 | CG | ARG | B | 847 | 27.486 | 25.797 | 33.273 | 1.00 | 46.99 |
| ATOM | 5120 | CD | ARG | B | 847 | 28.277 | 24.507 | 33.443 | 1.00 | 47.45 |
| ATOM | 5121 | NE | ARG | B | 847 | 29.724 | 24.700 | 33.406 | 1.00 | 47.23 |
| ATOM | 5122 | CZ | ARG | B | 847 | 30.594 | 23.737 | 33.111 | 1.00 | 47.33 |
| ATOM | 5123 | NH1 | ARG | B | 847 | 30.165 | 22.517 | 32.822 | 1.00 | 47.47 |
| ATOM | 5124 | NH2 | ARG | B | 847 | 31.896 | 23.989 | 33.109 | 1.00 | 46.97 |
| ATOM | 5125 | C | ARG | B | 847 | 25.893 | 28.301 | 35.776 | 1.00 | 26.91 |
| ATOM | 5126 | O | ARG | B | 847 | 25.046 | 27.842 | 36.549 | 1.00 | 27.77 |
| ATOM | 5127 | N | LYS | B | 848 | 26.592 | 29.400 | 36.039 | 1.00 | 28.28 |
| ATOM | 5128 | CA | LYS | B | 848 | 26.384 | 30.155 | 37.271 | 1.00 | 27.83 |
| ATOM | 5129 | CB | LYS | B | 848 | 27.420 | 31.277 | 37.399 | 1.00 | 32.07 |
| ATOM | 5130 | CG | LYS | B | 848 | 28.831 | 30.798 | 37.731 | 1.00 | 32.23 |
| ATOM | 5131 | CD | LYS | B | 848 | 29.798 | 31.975 | 37.858 | 1.00 | 32.55 |
| ATOM | 5132 | CE | LYS | B | 848 | 31.198 | 31.513 | 38.248 | 1.00 | 32.52 |
| ATOM | 5133 | NZ | LYS | B | 848 | 32.179 | 32.642 | 38.241 | 1.00 | 33.18 |
| ATOM | 5134 | C | LYS | B | 848 | 24.973 | 30.740 | 37.257 | 1.00 | 28.34 |
| ATOM | 5135 | O | LYS | B | 848 | 24.291 | 30.758 | 38.282 | 1.00 | 28.54 |
| ATOM | 5136 | N | ASN | B | 849 | 24.525 | 31.213 | 36.097 | 1.00 | 24.72 |
| ATOM | 5137 | CA | ASN | B | 849 | 23.179 | 31.765 | 36.026 | 1.00 | 24.90 |
| ATOM | 5138 | CB | ASN | B | 849 | 22.975 | 32.558 | 34.734 | 1.00 | 24.86 |
| ATOM | 5139 | CG | ASN | B | 849 | 23.561 | 33.949 | 34.817 | 1.00 | 25.77 |
| ATOM | 5140 | OD1 | ASN | B | 849 | 23.482 | 34.602 | 35.865 | 1.00 | 26.99 |
| ATOM | 5141 | ND2 | ASN | B | 849 | 24.139 | 34.422 | 33.717 | 1.00 | 25.44 |
| ATOM | 5142 | C | ASN | B | 849 | 22.117 | 30.676 | 36.149 | 1.00 | 24.90 |
| ATOM | 5143 | O | ASN | B | 849 | 21.041 | 30.909 | 36.698 | 1.00 | 25.65 |
| ATOM | 5144 | N | ARG | B | 850 | 22.410 | 29.484 | 35.639 | 1.00 | 26.25 |
| ATOM | 5145 | CA | ARG | B | 850 | 21.452 | 28.392 | 35.738 | 1.00 | 26.05 |
| ATOM | 5146 | CB | ARG | B | 850 | 21.991 | 27.128 | 35.071 | 1.00 | 37.42 |
| ATOM | 5147 | CG | ARG | B | 850 | 21.057 | 25.945 | 35.237 | 1.00 | 37.84 |
| ATOM | 5148 | CD | ARG | B | 850 | 21.721 | 24.635 | 34.879 | 1.00 | 37.92 |
| ATOM | 5149 | NE | ARG | B | 850 | 20.875 | 23.509 | 35.261 | 1.00 | 38.86 |
| ATOM | 5150 | CZ | ARG | B | 850 | 19.666 | 23.286 | 34.761 | 1.00 | 38.08 |
| ATOM | 5151 | NH1 | ARG | B | 850 | 19.168 | 24.114 | 33.854 | 1.00 | 39.16 |
| ATOM | 5152 | NH2 | ARG | B | 850 | 18.950 | 22.247 | 35.172 | 1.00 | 37.88 |
| ATOM | 5153 | C | ARG | B | 850 | 21.209 | 28.112 | 37.217 | 1.00 | 26.02 |
| ATOM | 5154 | O | ARG | B | 850 | 20.087 | 27.861 | 37.646 | 1.00 | 25.93 |
| ATOM | 5155 | N | GLN | B | 851 | 22.290 | 28.148 | 37.986 | 1.00 | 29.35 |
| ATOM | 5156 | CA | GLN | B | 851 | 22.231 | 27.915 | 39.419 | 1.00 | 29.80 |
| ATOM | 5157 | CB | GLN | B | 851 | 23.641 | 28.042 | 40.006 | 1.00 | 57.33 |
| ATOM | 5158 | CG | GLN | B | 851 | 23.706 | 28.453 | 41.467 | 1.00 | 58.18 |
| ATOM | 5159 | CD | GLN | B | 851 | 25.130 | 28.731 | 41.920 | 1.00 | 58.63 |
| ATOM | 5160 | OE1 | GLN | B | 851 | 25.355 | 29.314 | 42.983 | 1.00 | 58.50 |
| ATOM | 5161 | NE2 | GLN | B | 851 | 26.101 | 28.309 | 41.114 | 1.00 | 58.38 |
| ATOM | 5162 | C | GLN | B | 851 | 21.289 | 28.920 | 40.068 | 1.00 | 29.02 |
| ATOM | 5163 | O | GLN | B | 851 | 20.413 | 28.550 | 40.852 | 1.00 | 29.23 |
| ATOM | 5164 | N | LYS | B | 852 | 21.464 | 30.192 | 39.724 | 1.00 | 28.14 |
| ATOM | 5165 | CA | LYS | B | 852 | 20.635 | 31.253 | 40.283 | 1.00 | 28.58 |
| ATOM | 5166 | CB | LYS | B | 852 | 21.150 | 32.616 | 39.815 | 1.00 | 42.42 |
| ATOM | 5167 | CG | LYS | B | 852 | 22.495 | 33.007 | 40.409 | 1.00 | 42.77 |
| ATOM | 5168 | CD | LYS | B | 852 | 22.371 | 33.238 | 41.906 | 1.00 | 42.79 |
| ATOM | 5169 | CE | LYS | B | 852 | 23.700 | 33.633 | 42.527 | 1.00 | 43.26 |
| ATOM | 5170 | NZ | LYS | B | 852 | 23.538 | 34.014 | 43.959 | 1.00 | 43.16 |
| ATOM | 5171 | C | LYS | B | 852 | 19.153 | 31.116 | 39.941 | 1.00 | 28.34 |
| ATOM | 5172 | O | LYS | B | 852 | 18.296 | 31.254 | 40.819 | 1.00 | 28.28 |
| ATOM | 5173 | N | TRP | B | 853 | 18.846 | 30.854 | 38.672 | 1.00 | 28.99 |
| ATOM | 5174 | CA | TRP | B | 853 | 17.454 | 30.709 | 38.252 | 1.00 | 29.17 |
| ATOM | 5175 | CB | TRP | B | 853 | 17.355 | 30.629 | 36.719 | 1.00 | 25.14 |
| ATOM | 5176 | CG | TRP | B | 853 | 17.517 | 31.963 | 36.032 | 1.00 | 23.87 |
| ATOM | 5177 | CD2 | TRP | B | 853 | 16.695 | 33.121 | 36.210 | 1.00 | 23.86 |
| ATOM | 5178 | CE2 | TRP | B | 853 | 17.216 | 34.136 | 35.377 | 1.00 | 23.34 |
| ATOM | 5179 | CE3 | TRP | B | 853 | 15.567 | 33.400 | 36.995 | 1.00 | 24.36 |
| ATOM | 5180 | CD1 | TRP | B | 853 | 18.475 | 32.311 | 35.121 | 1.00 | 24.42 |
| ATOM | 5181 | NE1 | TRP | B | 853 | 18.302 | 33.614 | 34.725 | 1.00 | 24.36 |
| ATOM | 5182 | CZ2 | TRP | B | 853 | 16.648 | 35.412 | 35.305 | 1.00 | 23.69 |
| ATOM | 5183 | CZ3 | TRP | B | 853 | 15.002 | 34.669 | 36.924 | 1.00 | 23.64 |
| ATOM | 5184 | CH2 | TRP | B | 853 | 15.545 | 35.657 | 36.085 | 1.00 | 24.37 |
| ATOM | 5185 | C | TRP | B | 853 | 16.783 | 29.488 | 38.882 | 1.00 | 29.77 |
| ATOM | 5186 | O | TRP | B | 853 | 15.632 | 29.561 | 39.309 | 1.00 | 29.78 |
| ATOM | 5187 | N | GLN | B | 854 | 17.504 | 28.371 | 38.946 | 1.00 | 29.55 |
| ATOM | 5188 | CA | GLN | B | 854 | 16.951 | 27.150 | 39.526 | 1.00 | 30.02 |
| ATOM | 5189 | CB | GLN | B | 854 | 17.937 | 25.993 | 39.358 | 1.00 | 34.76 |
| ATOM | 5190 | CG | GLN | B | 854 | 17.451 | 24.662 | 39.918 | 1.00 | 34.86 |
| ATOM | 5191 | CD | GLN | B | 854 | 16.141 | 24.207 | 39.305 | 1.00 | 35.26 |
| ATOM | 5192 | OE1 | GLN | B | 854 | 15.984 | 24.197 | 38.085 | 1.00 | 35.00 |
| ATOM | 5193 | NE2 | GLN | B | 854 | 15.195 | 23.815 | 40.151 | 1.00 | 35.46 |
| ATOM | 5194 | C | GLN | B | 854 | 16.611 | 27.328 | 41.005 | 1.00 | 30.99 |
| ATOM | 5195 | O | GLN | B | 854 | 15.559 | 26.884 | 41.462 | 1.00 | 30.12 |
| ATOM | 5196 | N | ALA | B | 855 | 17.501 | 27.976 | 41.748 | 1.00 | 37.27 |
| ATOM | 5197 | CA | ALA | B | 855 | 17.270 | 28.206 | 43.171 | 1.00 | 38.24 |
| ATOM | 5198 | CB | ALA | B | 855 | 18.445 | 28.958 | 43.777 | 1.00 | 27.97 |
| ATOM | 5199 | C | ALA | B | 855 | 15.976 | 28.988 | 43.380 | 1.00 | 38.50 |
| ATOM | 5200 | O | ALA | B | 855 | 15.308 | 28.840 | 44.404 | 1.00 | 38.79 |
| ATOM | 5201 | N | LEU | B | 856 | 15.622 | 29.817 | 42.404 | 1.00 | 35.46 |
| ATOM | 5202 | CA | LEU | B | 856 | 14.398 | 30.613 | 42.484 | 1.00 | 35.13 |
| ATOM | 5203 | CB | LEU | B | 856 | 14.507 | 31.858 | 41.597 | 1.00 | 30.40 |
| ATOM | 5204 | CG | LEU | B | 856 | 15.541 | 32.924 | 41.965 | 1.00 | 29.57 |
| ATOM | 5205 | CD1 | LEU | B | 856 | 15.493 | 34.056 | 40.956 | 1.00 | 30.17 |
| ATOM | 5206 | CD2 | LEU | B | 856 | 15.266 | 33.443 | 43.367 | 1.00 | 30.22 |
| ATOM | 5207 | C | LEU | B | 856 | 13.179 | 29.805 | 42.054 | 1.00 | 34.94 |
| ATOM | 5208 | O | LEU | B | 856 | 12.076 | 30.012 | 42.555 | 1.00 | 34.78 |
| ATOM | 5209 | N | ALA | B | 857 | 13.385 | 28.876 | 41.127 | 1.00 | 36.42 |
| ATOM | 5210 | CA | ALA | B | 857 | 12.299 | 28.055 | 40.609 | 1.00 | 36.87 |
| ATOM | 5211 | CB | ALA | B | 857 | 12.648 | 27.577 | 39.206 | 1.00 | 33.84 |
| ATOM | 5212 | C | ALA | B | 857 | 11.926 | 26.859 | 41.482 | 1.00 | 37.18 |
| ATOM | 5213 | O | ALA | B | 857 | 10.774 | 26.429 | 41.482 | 1.00 | 37.43 |
| ATOM | 5214 | N | GLU | B | 858 | 12.892 | 26.319 | 42.216 | 1.00 | 41.64 |
| ATOM | 5215 | CA | GLU | B | 858 | 12.634 | 25.161 | 43.069 | 1.00 | 42.85 |
| ATOM | 5216 | CB | GLU | B | 858 | 13.921 | 24.368 | 43.295 | 1.00 | 56.15 |
| ATOM | 5217 | CG | GLU | B | 858 | 15.034 | 25.173 | 43.930 | 1.00 | 56.44 |
| ATOM | 5218 | CD | GLU | B | 858 | 16.249 | 24.329 | 44.252 | 1.00 | 56.70 |
| ATOM | 5219 | OE1 | GLU | B | 858 | 16.752 | 23.629 | 43.346 | 1.00 | 56.58 |
| ATOM | 5220 | OE2 | GLU | B | 858 | 16.700 | 24.370 | 45.415 | 1.00 | 56.71 |
| ATOM | 5221 | C | GLU | B | 858 | 12.037 | 25.541 | 44.416 | 1.00 | 42.83 |
| ATOM | 5222 | O | GLU | B | 858 | 11.799 | 26.744 | 44.645 | 1.00 | 42.70 |
| ATOM | 5223 | OXT | GLU | B | 858 | 11.814 | 24.618 | 45.227 | 1.00 | 56.34 |
| TER | | | | | | | | | | |
| ATOM | 5225 | N1 | SIL | A | 1 | −13.215 | −5.663 | 26.629 | 1.00 | 24.07 |
| ATOM | 5226 | C18 | SIL | A | 1 | −13.515 | −5.899 | 27.857 | 1.00 | 24.70 |
| ATOM | 5227 | C19 | SIL | A | 1 | −14.726 | −6.742 | 28.146 | 1.00 | 24.74 |
| ATOM | 5228 | C43 | SIL | A | 1 | −15.702 | −6.365 | 29.114 | 1.00 | 25.67 |
| ATOM | 5229 | O44 | SIL | A | 1 | −15.506 | −5.152 | 29.812 | 1.00 | 24.76 |
| ATOM | 5230 | C45 | SIL | A | 1 | −16.389 | −4.626 | 30.824 | 1.00 | 25.28 |
| ATOM | 5231 | C47 | SIL | A | 1 | −15.790 | −3.294 | 31.325 | 1.00 | 25.55 |
| ATOM | 5232 | C41 | SIL | A | 1 | −16.830 | −7.231 | 29.333 | 1.00 | 25.19 |
| ATOM | 5233 | C39 | SIL | A | 1 | −16.955 | −8.443 | 28.594 | 1.00 | 26.15 |
| ATOM | 5234 | C22 | SIL | A | 1 | −15.963 | −8.801 | 27.633 | 1.00 | 25.41 |
| ATOM | 5235 | S23 | SIL | A | 1 | −16.074 | −10.311 | 26.687 | 1.00 | 26.04 |
| ATOM | 5236 | O38 | SIL | A | 1 | −17.300 | −10.973 | 27.102 | 1.00 | 25.69 |
| ATOM | 5237 | O37 | SIL | A | 1 | −14.845 | −11.046 | 26.958 | 1.00 | 26.01 |
| ATOM | 5238 | N24 | SIL | A | 1 | −16.149 | −10.048 | 25.039 | 1.00 | 26.05 |
| ATOM | 5239 | C35 | SIL | A | 1 | −17.168 | −8.998 | 24.670 | 1.00 | 26.28 |
| ATOM | 5240 | C33 | SIL | A | 1 | −17.218 | −8.772 | 23.126 | 1.00 | 26.30 |
| ATOM | 5241 | N29 | SIL | A | 1 | −15.854 | −8.482 | 22.563 | 1.00 | 26.54 |
| ATOM | 5242 | C31 | SIL | A | 1 | −15.924 | −8.260 | 21.089 | 1.00 | 26.02 |
| ATOM | 5243 | C27 | SIL | A | 1 | −14.887 | −9.587 | 22.889 | 1.00 | 26.44 |
| ATOM | 5244 | C25 | SIL | A | 1 | −14.787 | −9.831 | 24.428 | 1.00 | 25.61 |
| ATOM | 5245 | C20 | SIL | A | 1 | −14.868 | −7.946 | 27.427 | 1.00 | 25.44 |
| ATOM | 5246 | N17 | SIL | A | 1 | −12.749 | −5.402 | 28.877 | 1.00 | 24.74 |
| ATOM | 5247 | C15 | SIL | A | 1 | −11.635 | −4.635 | 28.662 | 1.00 | 25.23 |
| ATOM | 5248 | O16 | SIL | A | 1 | −10.964 | −4.202 | 29.580 | 1.00 | 24.95 |
| ATOM | 5249 | C14 | SIL | A | 1 | −11.266 | −4.350 | 27.234 | 1.00 | 25.33 |
| ATOM | 5250 | N11 | SIL | A | 1 | −10.297 | −3.680 | 26.649 | 1.00 | 24.67 |
| ATOM | 5251 | C12 | SIL | A | 1 | −9.206 | −2.961 | 27.299 | 1.00 | 25.39 |
| ATOM | 5252 | N10 | SIL | A | 1 | −10.467 | −3.764 | 25.300 | 1.00 | 25.03 |
| ATOM | 5253 | C3 | SIL | A | 1 | −11.495 | −4.455 | 25.004 | 1.00 | 24.25 |
| ATOM | 5254 | C4 | SIL | A | 1 | −12.014 | −4.783 | 23.619 | 1.00 | 25.73 |
| ATOM | 5255 | C6 | SIL | A | 1 | −12.905 | −3.666 | 22.952 | 1.00 | 26.42 |
| ATOM | 5256 | C8 | SIL | A | 1 | −13.128 | −2.379 | 23.800 | 1.00 | 25.69 |
| ATOM | 5257 | C2 | SIL | A | 1 | −12.059 | −4.866 | 26.277 | 1.00 | 24.93 |
| TER | | | | | | | | | | |
| ATOM | 5259 | N1 | SIL | B | 1 | 15.164 | 33.812 | 25.541 | 1.00 | 25.10 |
| ATOM | 5260 | C18 | SIL | B | 1 | 14.895 | 33.565 | 26.769 | 1.00 | 24.27 |
| ATOM | 5261 | C19 | SIL | B | 1 | 13.700 | 32.702 | 27.070 | 1.00 | 24.84 |
| ATOM | 5262 | C43 | SIL | B | 1 | 12.729 | 33.045 | 28.058 | 1.00 | 24.56 |
| ATOM | 5263 | O44 | SIL | B | 1 | 12.899 | 34.250 | 28.780 | 1.00 | 26.57 |
| ATOM | 5264 | C45 | SIL | B | 1 | 12.006 | 34.722 | 29.810 | 1.00 | 25.69 |
| ATOM | 5265 | C47 | SIL | B | 1 | 12.558 | 36.064 | 30.336 | 1.00 | 25.09 |
| ATOM | 5266 | C41 | SIL | B | 1 | 11.622 | 32.157 | 28.280 | 1.00 | 25.59 |
| ATOM | 5267 | C39 | SIL | B | 1 | 11.507 | 30.960 | 27.526 | 1.00 | 25.77 |
| ATOM | 5268 | C22 | SIL | B | 1 | 12.487 | 30.633 | 26.544 | 1.00 | 25.79 |
| ATOM | 5269 | S23 | SIL | B | 1 | 12.368 | 29.137 | 25.587 | 1.00 | 28.36 |
| ATOM | 5270 | O38 | SIL | B | 1 | 11.164 | 28.457 | 26.018 | 1.00 | 26.14 |
| ATOM | 5271 | O37 | SIL | B | 1 | 13.594 | 28.402 | 25.827 | 1.00 | 26.31 |
| ATOM | 5272 | N24 | SIL | B | 1 | 12.244 | 29.398 | 23.942 | 1.00 | 26.57 |
| ATOM | 5273 | C35 | SIL | B | 1 | 11.267 | 30.495 | 23.609 | 1.00 | 25.93 |
| ATOM | 5274 | C33 | SIL | B | 1 | 11.168 | 30.716 | 22.066 | 1.00 | 26.61 |
| ATOM | 5275 | N29 | SIL | B | 1 | 12.519 | 30.913 | 21.439 | 1.00 | 26.95 |
| ATOM | 5276 | C31 | SIL | B | 1 | 12.402 | 31.132 | 19.969 | 1.00 | 25.72 |
| ATOM | 5277 | C27 | SIL | B | 1 | 13.436 | 29.763 | 21.739 | 1.00 | 26.60 |
| ATOM | 5278 | C25 | SIL | B | 1 | 13.583 | 29.525 | 23.276 | 1.00 | 26.54 |
| ATOM | 5279 | C20 | SIL | B | 1 | 13.569 | 31.507 | 26.332 | 1.00 | 24.95 |
| ATOM | 5280 | N17 | SIL | B | 1 | 15.678 | 34.063 | 27.775 | 1.00 | 25.01 |
| ATOM | 5281 | C15 | SIL | B | 1 | 16.778 | 34.843 | 27.541 | 1.00 | 25.62 |
| ATOM | 5282 | O16 | SIL | B | 1 | 17.471 | 35.279 | 28.442 | 1.00 | 25.56 |
| ATOM | 5283 | C14 | SIL | B | 1 | 17.109 | 35.142 | 26.111 | 1.00 | 24.85 |
| ATOM | 5284 | N11 | SIL | B | 1 | 18.054 | 35.828 | 25.516 | 1.00 | 24.72 |
| ATOM | 5285 | C12 | SIL | B | 1 | 19.147 | 36.556 | 26.153 | 1.00 | 25.78 |
| ATOM | 5286 | N10 | SIL | B | 1 | 17.863 | 35.755 | 24.172 | 1.00 | 25.21 |
| ATOM | 5287 | C3 | SIL | B | 1 | 16.841 | 35.056 | 23.885 | 1.00 | 25.43 |
| ATOM | 5288 | C4 | SIL | B | 1 | 16.304 | 34.734 | 22.500 | 1.00 | 25.15 |
| ATOM | 5289 | C6 | SIL | B | 1 | 15.454 | 35.880 | 21.819 | 1.00 | 26.50 |
| ATOM | 5290 | C8 | SIL | B | 1 | 15.284 | 37.187 | 22.646 | 1.00 | 26.25 |
| ATOM | 5291 | C2 | SIL | B | 1 | 16.305 | 34.627 | 25.167 | 1.00 | 24.69 |
| TER | | | | | | | | | | |
| ATOM | 5293 | ZN | ZN | A | 864 | −8.302 | 0.061 | 19.726 | 1.00 | 33.93 |
| ATOM | 5294 | MG | MG | A | 865 | −10.882 | −0.915 | 17.074 | 1.00 | 20.57 |
| TER | | | | | | | | | | |
| ATOM | 5296 | ZN | ZN | B | 864 | 19.969 | 39.499 | 18.508 | 1.00 | 25.29 |
| ATOM | 5297 | MG | MG | B | 865 | 0.014 | 0.254 | −1.283 | 1.00 | 32.82 |
| END | | | | | | | | | | |

TABLE 4
Atomic coordinates for PDE5* apo protein
Atom Number, Atom Type, Residue Type, Residue Number Cartesian
Coordinates X, Y, Z, Atom Occupancy (O), Temperature Factor (B)

| | | | | | | X | Y | Z | O | B |
|---|---|---|---|---|---|---|---|---|---|---|
| ATOM | 1 | CB | GLU | A | 536 | 9.887 | 25.182 | 24.787 | 1.00 | 33.98 |
| ATOM | 2 | CG | GLU | A | 536 | 11.305 | 25.697 | 24.635 | 1.00 | 42.32 |
| ATOM | 3 | CD | GLU | A | 536 | 11.492 | 27.074 | 25.245 | 1.00 | 47.12 |
| ATOM | 4 | OE1 | GLU | A | 536 | 11.290 | 27.223 | 26.470 | 1.00 | 54.45 |
| ATOM | 5 | OE2 | GLU | A | 536 | 11.842 | 28.012 | 24.499 | 1.00 | 51.15 |
| ATOM | 6 | C | GLU | A | 536 | 7.942 | 25.044 | 26.327 | 1.00 | 37.22 |
| ATOM | 7 | O | GLU | A | 536 | 7.222 | 24.272 | 25.690 | 1.00 | 36.76 |
| ATOM | 8 | N | GLU | A | 536 | 9.991 | 23.667 | 26.752 | 1.00 | 36.93 |
| ATOM | 9 | CA | GLU | A | 536 | 9.456 | 24.961 | 26.239 | 1.00 | 37.47 |
| ATOM | 10 | N | THR | A | 537 | 7.463 | 25.990 | 27.125 | 1.00 | 37.34 |
| ATOM | 11 | CA | THR | A | 537 | 6.035 | 26.189 | 27.289 | 1.00 | 36.78 |
| ATOM | 12 | CB | THR | A | 537 | 5.750 | 27.235 | 28.387 | 1.00 | 43.61 |
| ATOM | 13 | OG1 | THR | A | 537 | 6.425 | 28.457 | 28.069 | 1.00 | 45.23 |
| ATOM | 14 | CG2 | THR | A | 537 | 6.241 | 26.731 | 29.739 | 1.00 | 34.90 |
| ATOM | 15 | C | THR | A | 537 | 5.452 | 26.660 | 25.957 | 1.00 | 35.15 |
| ATOM | 16 | O | THR | A | 537 | 4.239 | 26.661 | 25.764 | 1.00 | 34.30 |
| ATOM | 17 | N | ARG | A | 538 | 6.336 | 27.046 | 25.040 | 1.00 | 32.72 |
| ATOM | 18 | CA | ARG | A | 538 | 5.943 | 27.514 | 23.712 | 1.00 | 31.35 |
| ATOM | 19 | CB | ARG | A | 538 | 7.143 | 28.133 | 23.002 | 1.00 | 33.78 |
| ATOM | 20 | CG | ARG | A | 538 | 6.910 | 28.452 | 21.531 | 1.00 | 42.77 |
| ATOM | 21 | CD | ARG | A | 538 | 6.041 | 29.690 | 21.340 | 1.00 | 51.85 |
| ATOM | 22 | NE | ARG | A | 538 | 4.638 | 29.475 | 21.687 | 1.00 | 50.21 |
| ATOM | 23 | CZ | ARG | A | 538 | 3.715 | 30.433 | 21.672 | 1.00 | 49.57 |
| ATOM | 24 | NH1 | ARG | A | 538 | 4.047 | 31.673 | 21.330 | 1.00 | 48.08 |
| ATOM | 25 | NH2 | ARG | A | 538 | 2.458 | 30.154 | 21.992 | 1.00 | 49.14 |
| ATOM | 26 | C | ARG | A | 538 | 5.405 | 26.369 | 22.861 | 1.00 | 30.33 |
| ATOM | 27 | O | ARG | A | 538 | 4.381 | 26.501 | 22.187 | 1.00 | 28.72 |
| ATOM | 28 | N | GLU | A | 539 | 6.120 | 25.250 | 22.876 | 1.00 | 28.94 |
| ATOM | 29 | CA | GLU | A | 539 | 5.709 | 24.080 | 22.115 | 1.00 | 25.47 |
| ATOM | 30 | CB | GLU | A | 539 | 6.807 | 23.024 | 22.141 | 1.00 | 30.35 |
| ATOM | 31 | CG | GLU | A | 539 | 8.055 | 23.426 | 21.391 | 1.00 | 31.75 |
| ATOM | 32 | CD | GLU | A | 539 | 9.188 | 22.463 | 21.628 | 1.00 | 31.48 |
| ATOM | 33 | OE1 | GLU | A | 539 | 9.689 | 22.410 | 22.771 | 1.00 | 46.55 |
| ATOM | 34 | OE2 | GLU | A | 539 | 9.575 | 21.754 | 20.676 | 1.00 | 57.06 |
| ATOM | 35 | C | GLU | A | 539 | 4.437 | 23.511 | 22.718 | 1.00 | 23.49 |
| ATOM | 36 | O | GLU | A | 539 | 3.599 | 22.952 | 22.011 | 1.00 | 21.01 |
| ATOM | 37 | N | LEU | A | 540 | 4.299 | 23.653 | 24.032 | 1.00 | 22.37 |
| ATOM | 38 | CA | LEU | A | 540 | 3.114 | 23.159 | 24.713 | 1.00 | 22.10 |
| ATOM | 39 | CB | LEU | A | 540 | 3.302 | 23.223 | 26.231 | 1.00 | 19.44 |
| ATOM | 40 | CG | LEU | A | 540 | 2.098 | 22.763 | 27.059 | 1.00 | 22.04 |
| ATOM | 41 | CD1 | LEU | A | 540 | 1.686 | 21.365 | 26.627 | 1.00 | 19.81 |
| ATOM | 42 | CD2 | LEU | A | 540 | 2.441 | 22.783 | 28.539 | 1.00 | 38.12 |
| ATOM | 43 | C | LEU | A | 540 | 1.907 | 23.995 | 24.300 | 1.00 | 21.23 |
| ATOM | 44 | O | LEU | A | 540 | 0.844 | 23.461 | 23.990 | 1.00 | 19.37 |
| ATOM | 45 | N | GLN | A | 541 | 2.074 | 25.313 | 24.283 | 1.00 | 20.36 |
| ATOM | 46 | CA | GLN | A | 541 | 0.972 | 26.185 | 23.905 | 1.00 | 18.42 |
| ATOM | 47 | CB | GLN | A | 541 | 1.410 | 27.651 | 23.966 | 1.00 | 20.67 |
| ATOM | 48 | CG | GLN | A | 541 | 1.873 | 28.081 | 25.350 | 1.00 | 24.12 |
| ATOM | 49 | CD | GLN | A | 541 | 2.356 | 29.519 | 25.401 | 1.00 | 24.37 |
| ATOM | 50 | OE1 | GLN | A | 541 | 3.047 | 29.986 | 24.497 | 1.00 | 17.86 |
| ATOM | 51 | NE2 | GLN | A | 541 | 2.008 | 30.223 | 26.474 | 1.00 | 20.12 |
| ATOM | 52 | C | GLN | A | 541 | 0.477 | 25.833 | 22.505 | 1.00 | 17.74 |
| ATOM | 53 | O | GLN | A | 541 | −0.726 | 25.799 | 22.254 | 1.00 | 19.08 |
| ATOM | 54 | N | SER | A | 542 | 1.404 | 25.552 | 21.596 | 1.00 | 15.82 |
| ATOM | 55 | CA | SER | A | 542 | 1.026 | 25.207 | 20.231 | 1.00 | 18.42 |
| ATOM | 56 | CB | SER | A | 542 | 2.263 | 25.179 | 19.329 | 1.00 | 20.91 |
| ATOM | 57 | OG | SER | A | 542 | 2.857 | 26.463 | 19.251 | 1.00 | 37.22 |
| ATOM | 58 | C | SER | A | 542 | 0.320 | 23.858 | 20.174 | 1.00 | 16.82 |
| ATOM | 59 | O | SER | A | 542 | −0.710 | 23.709 | 19.520 | 1.00 | 17.71 |
| ATOM | 60 | N | LEU | A | 543 | 0.875 | 22.874 | 20.872 | 1.00 | 17.64 |
| ATOM | 61 | CA | LEU | A | 543 | 0.297 | 21.538 | 20.878 | 1.00 | 17.88 |
| ATOM | 62 | CB | LEU | A | 543 | 1.269 | 20.550 | 21.531 | 1.00 | 13.54 |
| ATOM | 63 | CG | LEU | A | 543 | 0.762 | 19.113 | 21.695 | 1.00 | 17.02 |
| ATOM | 64 | CD1 | LEU | A | 543 | 0.501 | 18.504 | 20.329 | 1.00 | 14.15 |
| ATOM | 65 | CD2 | LEU | A | 543 | 1.789 | 18.284 | 22.465 | 1.00 | 10.59 |
| ATOM | 66 | C | LEU | A | 543 | −1.049 | 21.468 | 21.592 | 1.00 | 18.33 |
| ATOM | 67 | O | LEU | A | 543 | −2.013 | 20.914 | 21.064 | 1.00 | 19.18 |
| ATOM | 68 | N | ALA | A | 544 | −1.112 | 22.038 | 22.790 | 1.00 | 18.60 |
| ATOM | 69 | CA | ALA | A | 544 | −2.332 | 22.003 | 23.588 | 1.00 | 19.09 |
| ATOM | 70 | CB | ALA | A | 544 | −2.053 | 22.573 | 24.972 | 1.00 | 17.83 |
| ATOM | 71 | C | ALA | A | 544 | −3.527 | 22.717 | 22.967 | 1.00 | 21.56 |
| ATOM | 72 | O | ALA | A | 544 | −4.667 | 22.306 | 23.165 | 1.00 | 23.51 |
| ATOM | 73 | N | ALA | A | 545 | −3.269 | 23.782 | 22.217 | 1.00 | 23.98 |
| ATOM | 74 | CA | ALA | A | 545 | −4.348 | 24.547 | 21.599 | 1.00 | 24.34 |
| ATOM | 75 | CB | ALA | A | 545 | −3.861 | 25.952 | 21.274 | 1.00 | 26.53 |
| ATOM | 76 | C | ALA | A | 545 | −4.901 | 23.891 | 20.341 | 1.00 | 25.76 |
| ATOM | 77 | O | ALA | A | 545 | −6.065 | 24.079 | 19.992 | 1.00 | 28.16 |
| ATOM | 78 | N | ALA | A | 546 | −4.059 | 23.115 | 19.668 | 1.00 | 24.74 |
| ATOM | 79 | CA | ALA | A | 546 | −4.441 | 22.443 | 18.430 | 1.00 | 24.22 |
| ATOM | 80 | CB | ALA | A | 546 | −3.239 | 21.701 | 17.861 | 1.00 | 22.00 |
| ATOM | 81 | C | ALA | A | 546 | −5.621 | 21.485 | 18.529 | 1.00 | 21.89 |
| ATOM | 82 | O | ALA | A | 546 | −5.803 | 20.791 | 19.529 | 1.00 | 23.27 |
| ATOM | 83 | N | VAL | A | 547 | −6.433 | 21.464 | 17.478 | 1.00 | 21.72 |
| ATOM | 84 | CA | VAL | A | 547 | −7.560 | 20.548 | 17.418 | 1.00 | 21.65 |
| ATOM | 85 | CB | VAL | A | 547 | −8.660 | 21.045 | 16.454 | 1.00 | 23.33 |
| ATOM | 86 | CG1 | VAL | A | 547 | −9.754 | 19.993 | 16.326 | 1.00 | 25.11 |
| ATOM | 87 | CG2 | VAL | A | 547 | −9.250 | 22.355 | 16.965 | 1.00 | 23.58 |
| ATOM | 88 | C | VAL | A | 547 | −6.925 | 19.284 | 16.850 | 1.00 | 22.28 |
| ATOM | 89 | O | VAL | A | 547 | −6.280 | 19.329 | 15.805 | 1.00 | 24.96 |
| ATOM | 90 | N | VAL | A | 548 | −7.090 | 18.165 | 17.542 | 1.00 | 21.82 |
| ATOM | 91 | CA | VAL | A | 548 | −6.498 | 16.909 | 17.098 | 1.00 | 19.25 |
| ATOM | 92 | CB | VAL | A | 548 | −6.387 | 15.925 | 18.278 | 1.00 | 19.56 |
| ATOM | 93 | CG1 | VAL | A | 548 | −5.669 | 14.659 | 17.840 | 1.00 | 23.78 |
| ATOM | 94 | CG2 | VAL | A | 548 | −5.656 | 16.593 | 19.438 | 1.00 | 24.95 |
| ATOM | 95 | C | VAL | A | 548 | −7.284 | 16.241 | 15.975 | 1.00 | 17.26 |
| ATOM | 96 | O | VAL | A | 548 | −8.453 | 15.904 | 16.146 | 1.00 | 19.24 |
| ATOM | 97 | N | PRO | A | 549 | −6.646 | 16.038 | 14.808 | 1.00 | 17.33 |
| ATOM | 98 | CD | PRO | A | 549 | −5.293 | 16.478 | 14.431 | 1.00 | 18.64 |
| ATOM | 99 | CA | PRO | A | 549 | −7.315 | 15.401 | 13.670 | 1.00 | 19.43 |
| ATOM | 100 | CB | PRO | A | 549 | −6.245 | 15.425 | 12.575 | 1.00 | 17.30 |
| ATOM | 101 | CG | PRO | A | 549 | −5.420 | 16.615 | 12.931 | 1.00 | 23.24 |
| ATOM | 102 | C | PRO | A | 549 | −7.742 | 13.982 | 14.023 | 1.00 | 20.23 |
| ATOM | 103 | O | PRO | A | 549 | −7.259 | 13.402 | 15.001 | 1.00 | 16.82 |
| ATOM | 104 | N | SER | A | 550 | −8.645 | 13.431 | 13.220 | 1.00 | 17.57 |
| ATOM | 105 | CA | SER | A | 550 | −9.153 | 12.081 | 13.432 | 1.00 | 20.68 |
| ATOM | 106 | CB | SER | A | 550 | −10.343 | 11.822 | 12.512 | 1.00 | 17.19 |
| ATOM | 107 | OG | SER | A | 550 | −9.934 | 11.861 | 11.153 | 1.00 | 18.60 |
| ATOM | 108 | C | SER | A | 550 | −8.075 | 11.055 | 13.127 | 1.00 | 17.34 |
| ATOM | 109 | O | SER | A | 550 | −7.096 | 11.357 | 12.443 | 1.00 | 16.71 |
| ATOM | 110 | N | ALA | A | 551 | −8.257 | 9.841 | 13.637 | 1.00 | 18.46 |
| ATOM | 111 | CA | ALA | A | 551 | −7.304 | 8.769 | 13.380 | 1.00 | 19.34 |
| ATOM | 112 | CB | ALA | A | 551 | −7.706 | 7.507 | 14.143 | 1.00 | 17.06 |
| ATOM | 113 | C | ALA | A | 551 | −7.322 | 8.502 | 11.879 | 1.00 | 18.62 |
| ATOM | 114 | O | ALA | A | 551 | −6.284 | 8.262 | 11.261 | 1.00 | 20.73 |
| ATOM | 115 | N | GLN | A | 552 | −8.518 | 8.554 | 11.300 | 1.00 | 19.78 |
| ATOM | 116 | CA | GLN | A | 552 | −8.700 | 8.327 | 9.869 | 1.00 | 21.36 |
| ATOM | 117 | CB | GLN | A | 552 | −10.175 | 8.528 | 9.502 | 1.00 | 23.40 |
| ATOM | 118 | CG | GLN | A | 552 | −10.541 | 8.181 | 8.067 | 1.00 | 31.79 |
| ATOM | 119 | CD | GLN | A | 552 | −12.026 | 8.358 | 7.797 | 1.00 | 35.80 |
| ATOM | 120 | OE1 | GLN | A | 552 | −12.864 | 7.675 | 8.390 | 1.00 | 46.36 |
| ATOM | 121 | NE2 | GLN | A | 552 | −12.358 | 9.283 | 6.902 | 1.00 | 49.03 |
| ATOM | 122 | C | GLN | A | 552 | −7.820 | 9.301 | 9.086 | 1.00 | 21.35 |
| ATOM | 123 | O | GLN | A | 552 | −7.040 | 8.897 | 8.219 | 1.00 | 21.48 |
| ATOM | 124 | N | THR | A | 553 | −7.938 | 10.584 | 9.408 | 1.00 | 19.75 |
| ATOM | 125 | CA | THR | A | 553 | −7.151 | 11.612 | 8.739 | 1.00 | 19.52 |
| ATOM | 126 | CB | THR | A | 553 | −7.501 | 13.017 | 9.268 | 1.00 | 18.18 |
| ATOM | 127 | OG1 | THR | A | 553 | −8.871 | 13.318 | 8.971 | 1.00 | 19.96 |
| ATOM | 128 | CG2 | THR | A | 553 | −6.597 | 14.066 | 8.627 | 1.00 | 18.92 |
| ATOM | 129 | C | THR | A | 553 | −5.654 | 11.396 | 8.926 | 1.00 | 20.00 |
| ATOM | 130 | O | THR | A | 553 | −4.875 | 11.523 | 7.980 | 1.00 | 19.53 |
| ATOM | 131 | N | LEU | A | 554 | −5.260 | 11.062 | 10.150 | 1.00 | 19.10 |
| ATOM | 132 | CA | LEU | A | 554 | −3.855 | 10.853 | 10.474 | 1.00 | 20.03 |
| ATOM | 133 | CB | LEU | A | 554 | −3.650 | 10.974 | 11.987 | 1.00 | 17.91 |
| ATOM | 134 | CG | LEU | A | 554 | −4.091 | 12.323 | 12.571 | 1.00 | 18.40 |
| ATOM | 135 | CD1 | LEU | A | 554 | −3.972 | 12.316 | 14.082 | 1.00 | 16.11 |
| ATOM | 136 | CD2 | LEU | A | 554 | −3.237 | 13.436 | 11.977 | 1.00 | 23.68 |
| ATOM | 137 | C | LEU | A | 554 | −3.303 | 9.521 | 9.970 | 1.00 | 19.88 |
| ATOM | 138 | O | LEU | A | 554 | −2.093 | 9.304 | 9.994 | 1.00 | 19.74 |
| ATOM | 139 | N | LYS | A | 555 | −4.198 | 8.637 | 9.533 | 1.00 | 17.37 |
| ATOM | 140 | CA | LYS | A | 555 | −3.833 | 7.327 | 8.997 | 1.00 | 19.69 |
| ATOM | 141 | CB | LYS | A | 555 | −3.032 | 7.523 | 7.708 | 1.00 | 26.45 |
| ATOM | 142 | CG | LYS | A | 555 | −3.740 | 8.429 | 6.708 | 1.00 | 33.54 |
| ATOM | 143 | CD | LYS | A | 555 | −2.829 | 8.837 | 5.565 | 1.00 | 36.95 |
| ATOM | 144 | CE | LYS | A | 555 | −3.542 | 9.803 | 4.633 | 1.00 | 36.53 |
| ATOM | 145 | NZ | LYS | A | 555 | −2.645 | 10.308 | 3.562 | 1.00 | 43.43 |
| ATOM | 146 | C | LYS | A | 555 | −3.043 | 6.454 | 9.976 | 1.00 | 19.45 |
| ATOM | 147 | O | LYS | A | 555 | −2.294 | 5.569 | 9.565 | 1.00 | 18.29 |
| ATOM | 148 | N | ILE | A | 556 | −3.227 | 6.688 | 11.271 | 1.00 | 17.06 |
| ATOM | 149 | CA | ILE | A | 556 | −2.501 | 5.917 | 12.273 | 1.00 | 16.83 |
| ATOM | 150 | CB | ILE | A | 556 | −2.475 | 6.638 | 13.641 | 1.00 | 13.95 |
| ATOM | 151 | CG2 | ILE | A | 556 | −1.565 | 7.856 | 13.569 | 1.00 | 16.87 |
| ATOM | 152 | CG1 | ILE | A | 556 | −3.897 | 7.017 | 14.062 | 1.00 | 14.93 |
| ATOM | 153 | CD1 | ILE | A | 556 | −3.981 | 7.598 | 15.460 | 1.00 | 19.73 |
| ATOM | 154 | C | ILE | A | 556 | −3.019 | 4.496 | 12.489 | 1.00 | 14.60 |
| ATOM | 155 | O | ILE | A | 556 | −2.443 | 3.745 | 13.271 | 1.00 | 12.74 |
| ATOM | 156 | N | THR | A | 557 | −4.099 | 4.125 | 11.806 | 1.00 | 14.27 |
| ATOM | 157 | CA | THR | A | 557 | −4.653 | 2.776 | 11.938 | 1.00 | 15.05 |
| ATOM | 158 | CB | THR | A | 557 | −6.187 | 2.772 | 11.699 | 1.00 | 21.22 |
| ATOM | 159 | OG1 | THR | A | 557 | −6.813 | 3.726 | 12.567 | 1.00 | 20.77 |
| ATOM | 160 | CG2 | THR | A | 557 | −6.773 | 1.398 | 11.982 | 1.00 | 21.26 |
| ATOM | 161 | C | THR | A | 557 | −3.991 | 1.855 | 10.907 | 1.00 | 14.39 |
| ATOM | 162 | O | THR | A | 557 | −4.131 | 0.631 | 10.955 | 1.00 | 16.89 |
| ATOM | 163 | N | ASP | A | 558 | −3.257 | 2.456 | 9.979 | 1.00 | 14.56 |
| ATOM | 164 | CA | ASP | A | 558 | −2.593 | 1.702 | 8.923 | 1.00 | 14.77 |
| ATOM | 165 | CB | ASP | A | 558 | −2.374 | 2.613 | 7.713 | 1.00 | 20.77 |
| ATOM | 166 | CG | ASP | A | 558 | −3.648 | 3.312 | 7.274 | 1.00 | 30.09 |
| ATOM | 167 | OD1 | ASP | A | 558 | −4.657 | 2.616 | 7.031 | 1.00 | 38.75 |
| ATOM | 168 | OD2 | ASP | A | 558 | −3.642 | 4.555 | 7.173 | 1.00 | 46.69 |
| ATOM | 169 | C | ASP | A | 558 | −1.258 | 1.086 | 9.351 | 1.00 | 14.68 |
| ATOM | 170 | O | ASP | A | 558 | −0.365 | 1.787 | 9.829 | 1.00 | 13.87 |
| ATOM | 171 | N | PHE | A | 559 | −1.124 | −0.227 | 9.175 | 1.00 | 13.82 |
| ATOM | 172 | CA | PHE | A | 559 | 0.119 | −0.916 | 9.526 | 1.00 | 13.07 |
| ATOM | 173 | CB | PHE | A | 559 | 0.000 | −2.427 | 9.283 | 1.00 | 14.41 |
| ATOM | 174 | CG | PHE | A | 559 | −0.732 | −3.175 | 10.366 | 1.00 | 18.21 |
| ATOM | 175 | CD1 | PHE | A | 559 | −0.289 | −3.134 | 11.680 | 1.00 | 14.30 |
| ATOM | 176 | CD2 | PHE | A | 559 | −1.836 | −3.960 | 10.060 | 1.00 | 16.61 |
| ATOM | 177 | CE1 | PHE | A | 559 | −0.931 | −3.866 | 12.668 | 1.00 | 15.41 |
| ATOM | 178 | CE2 | PHE | A | 559 | −2.482 | −4.692 | 11.043 | 1.00 | 15.41 |
| ATOM | 179 | CZ | PHE | A | 559 | −2.031 | −4.647 | 12.344 | 1.00 | 12.10 |
| ATOM | 180 | C | PHE | A | 559 | 1.277 | −0.386 | 8.680 | 1.00 | 11.71 |
| ATOM | 181 | O | PHE | A | 559 | 2.431 | −0.411 | 9.109 | 1.00 | 12.25 |
| ATOM | 182 | N | SER | A | 560 | 0.965 | 0.079 | 7.473 | 1.00 | 13.82 |
| ATOM | 183 | CA | SER | A | 560 | 1.979 | 0.597 | 6.555 | 1.00 | 13.86 |
| ATOM | 184 | CB | SER | A | 560 | 1.538 | 0.384 | 5.106 | 1.00 | 19.52 |
| ATOM | 185 | OG | SER | A | 560 | 1.411 | −0.996 | 4.816 | 1.00 | 19.35 |
| ATOM | 186 | C | SER | A | 560 | 2.294 | 2.071 | 6.770 | 1.00 | 12.88 |
| ATOM | 187 | O | SER | A | 560 | 2.959 | 2.698 | 5.945 | 1.00 | 14.29 |
| ATOM | 188 | N | PHE | A | 561 | 1.815 | 2.608 | 7.886 | 1.00 | 12.82 |
| ATOM | 189 | CA | PHE | A | 561 | 2.021 | 4.009 | 8.261 | 1.00 | 13.63 |
| ATOM | 190 | CB | PHE | A | 561 | 1.567 | 4.207 | 9.713 | 1.00 | 14.59 |
| ATOM | 191 | CG | PHE | A | 561 | 1.835 | 5.583 | 10.258 | 1.00 | 12.08 |
| ATOM | 192 | CD1 | PHE | A | 561 | 0.956 | 6.625 | 10.014 | 1.00 | 17.74 |
| ATOM | 193 | CD2 | PHE | A | 561 | 2.967 | 5.829 | 11.022 | 1.00 | 10.31 |
| ATOM | 194 | CE1 | PHE | A | 561 | 1.199 | 7.891 | 10.524 | 1.00 | 21.75 |
| ATOM | 195 | CE2 | PHE | A | 561 | 3.220 | 7.093 | 11.537 | 1.00 | 13.27 |
| ATOM | 196 | CZ | PHE | A | 561 | 2.333 | 8.126 | 11.287 | 1.00 | 18.85 |
| ATOM | 197 | C | PHE | A | 561 | 3.466 | 4.498 | 8.125 | 1.00 | 14.38 |
| ATOM | 198 | O | PHE | A | 561 | 4.413 | 3.791 | 8.485 | 1.00 | 11.10 |
| ATOM | 199 | N | SER | A | 562 | 3.625 | 5.716 | 7.607 | 1.00 | 14.38 |
| ATOM | 200 | CA | SER | A | 562 | 4.939 | 6.334 | 7.453 | 1.00 | 13.72 |
| ATOM | 201 | CB | SER | A | 562 | 5.283 | 6.520 | 5.972 | 1.00 | 15.96 |
| ATOM | 202 | OG | SER | A | 562 | 6.542 | 7.159 | 5.835 | 1.00 | 23.25 |
| ATOM | 203 | C | SER | A | 562 | 4.940 | 7.695 | 8.159 | 1.00 | 12.53 |
| ATOM | 204 | O | SER | A | 562 | 4.009 | 8.490 | 8.014 | 1.00 | 12.55 |
| ATOM | 205 | N | ASP | A | 563 | 5.992 | 7.965 | 8.922 | 1.00 | 13.45 |
| ATOM | 206 | CA | ASP | A | 563 | 6.094 | 9.223 | 9.661 | 1.00 | 15.79 |
| ATOM | 207 | CB | ASP | A | 563 | 6.662 | 8.953 | 11.050 | 1.00 | 13.73 |
| ATOM | 208 | CG | ASP | A | 563 | 8.166 | 8.765 | 11.022 | 1.00 | 15.49 |
| ATOM | 209 | OD1 | ASP | A | 563 | 8.895 | 9.680 | 11.465 | 1.00 | 18.81 |
| ATOM | 210 | OD2 | ASP | A | 563 | 8.625 | 7.712 | 10.537 | 1.00 | 16.12 |
| ATOM | 211 | C | ASP | A | 563 | 7.009 | 10.232 | 8.974 | 1.00 | 16.63 |
| ATOM | 212 | O | ASP | A | 563 | 7.112 | 11.383 | 9.404 | 1.00 | 17.82 |
| ATOM | 213 | N | PHE | A | 564 | 7.673 | 9.796 | 7.911 | 1.00 | 16.51 |
| ATOM | 214 | CA | PHE | A | 564 | 8.633 | 10.635 | 7.205 | 1.00 | 18.23 |
| ATOM | 215 | CB | PHE | A | 564 | 9.207 | 9.871 | 6.011 | 1.00 | 15.87 |
| ATOM | 216 | CG | PHE | A | 564 | 10.446 | 10.494 | 5.438 | 1.00 | 26.61 |
| ATOM | 217 | CD1 | PHE | A | 564 | 11.599 | 10.592 | 6.201 | 1.00 | 20.64 |
| ATOM | 218 | CD2 | PHE | A | 564 | 10.458 | 10.991 | 4.144 | 1.00 | 27.32 |
| ATOM | 219 | CE1 | PHE | A | 564 | 12.742 | 11.174 | 5.685 | 1.00 | 30.38 |
| ATOM | 220 | CE2 | PHE | A | 564 | 11.599 | 11.574 | 3.622 | 1.00 | 33.45 |
| ATOM | 221 | CZ | PHE | A | 564 | 12.742 | 11.665 | 4.394 | 1.00 | 33.80 |
| ATOM | 222 | C | PHE | A | 564 | 8.157 | 12.010 | 6.740 | 1.00 | 18.52 |
| ATOM | 223 | O | PHE | A | 564 | 8.942 | 12.959 | 6.728 | 1.00 | 19.54 |
| ATOM | 224 | N | GLU | A | 565 | 6.885 | 12.122 | 6.369 | 1.00 | 16.14 |
| ATOM | 225 | CA | GLU | A | 565 | 6.340 | 13.391 | 5.890 | 1.00 | 19.03 |
| ATOM | 226 | CB | GLU | A | 565 | 5.277 | 13.142 | 4.813 | 1.00 | 18.54 |
| ATOM | 227 | CG | GLU | A | 565 | 5.708 | 12.238 | 3.668 | 1.00 | 33.46 |
| ATOM | 228 | CD | GLU | A | 565 | 6.004 | 10.818 | 4.117 | 1.00 | 40.92 |
| ATOM | 229 | OE1 | GLU | A | 565 | 5.205 | 10.258 | 4.899 | 1.00 | 41.02 |
| ATOM | 230 | OE2 | GLU | A | 565 | 7.032 | 10.259 | 3.680 | 1.00 | 50.71 |
| ATOM | 231 | C | GLU | A | 565 | 5.710 | 14.229 | 7.002 | 1.00 | 19.43 |
| ATOM | 232 | O | GLU | A | 565 | 5.158 | 15.301 | 6.740 | 1.00 | 20.33 |
| ATOM | 233 | N | LEU | A | 566 | 5.786 | 13.746 | 8.238 | 1.00 | 13.91 |
| ATOM | 234 | CA | LEU | A | 566 | 5.193 | 14.463 | 9.362 | 1.00 | 15.26 |
| ATOM | 235 | CB | LEU | A | 566 | 4.515 | 13.482 | 10.321 | 1.00 | 15.63 |
| ATOM | 236 | CG | LEU | A | 566 | 3.329 | 12.669 | 9.799 | 1.00 | 24.63 |
| ATOM | 237 | CD1 | LEU | A | 566 | 2.864 | 11.698 | 10.881 | 1.00 | 23.72 |
| ATOM | 238 | CD2 | LEU | A | 566 | 2.199 | 13.607 | 9.399 | 1.00 | 33.15 |
| ATOM | 239 | C | LEU | A | 566 | 6.187 | 15.306 | 10.147 | 1.00 | 14.43 |
| ATOM | 240 | O | LEU | A | 566 | 7.358 | 14.960 | 10.265 | 1.00 | 17.81 |
| ATOM | 241 | N | SER | A | 567 | 5.699 | 16.419 | 10.685 | 1.00 | 14.78 |
| ATOM | 242 | CA | SER | A | 567 | 6.517 | 17.317 | 11.486 | 1.00 | 14.98 |
| ATOM | 243 | CB | SER | A | 567 | 5.928 | 18.727 | 11.467 | 1.00 | 21.89 |
| ATOM | 244 | OG | SER | A | 567 | 4.679 | 18.752 | 12.144 | 1.00 | 17.63 |
| ATOM | 245 | C | SER | A | 567 | 6.492 | 16.797 | 12.922 | 1.00 | 13.89 |
| ATOM | 246 | O | SER | A | 567 | 5.654 | 15.963 | 13.268 | 1.00 | 13.82 |
| ATOM | 247 | N | ASP | A | 568 | 7.403 | 17.289 | 13.753 | 1.00 | 15.43 |
| ATOM | 248 | CA | ASP | A | 568 | 7.428 | 16.869 | 15.149 | 1.00 | 15.57 |
| ATOM | 249 | CB | ASP | A | 568 | 8.536 | 17.603 | 15.904 | 1.00 | 19.60 |
| ATOM | 250 | CG | ASP | A | 568 | 9.888 | 16.947 | 15.727 | 1.00 | 16.75 |
| ATOM | 251 | OD1 | ASP | A | 568 | 9.989 | 16.021 | 14.899 | 1.00 | 19.05 |
| ATOM | 252 | OD2 | ASP | A | 568 | 10.848 | 17.355 | 16.415 | 1.00 | 23.47 |
| ATOM | 253 | C | ASP | A | 568 | 6.076 | 17.145 | 15.805 | 1.00 | 16.52 |
| ATOM | 254 | O | ASP | A | 568 | 5.559 | 16.313 | 16.556 | 1.00 | 15.97 |
| ATOM | 255 | N | LEU | A | 569 | 5.505 | 18.311 | 15.516 | 1.00 | 15.09 |
| ATOM | 256 | CA | LEU | A | 569 | 4.213 | 18.683 | 16.076 | 1.00 | 15.08 |
| ATOM | 257 | CB | LEU | A | 569 | 3.815 | 20.093 | 15.620 | 1.00 | 16.05 |
| ATOM | 258 | CG | LEU | A | 569 | 2.385 | 20.516 | 15.974 | 1.00 | 23.47 |
| ATOM | 259 | CD1 | LEU | A | 569 | 2.203 | 20.527 | 17.489 | 1.00 | 21.65 |
| ATOM | 260 | CD2 | LEU | A | 569 | 2.103 | 21.896 | 15.398 | 1.00 | 27.54 |
| ATOM | 261 | C | LEU | A | 569 | 3.136 | 17.694 | 15.647 | 1.00 | 12.90 |
| ATOM | 262 | O | LEU | A | 569 | 2.279 | 17.308 | 16.441 | 1.00 | 16.26 |
| ATOM | 263 | N | GLU | A | 570 | 3.184 | 17.281 | 14.386 | 1.00 | 13.40 |
| ATOM | 264 | CA | GLU | A | 570 | 2.202 | 16.340 | 13.869 | 1.00 | 14.08 |
| ATOM | 265 | CB | GLU | A | 570 | 2.354 | 16.196 | 12.352 | 1.00 | 14.43 |
| ATOM | 266 | CG | GLU | A | 570 | 1.885 | 17.432 | 11.579 | 1.00 | 20.85 |
| ATOM | 267 | CD | GLU | A | 570 | 2.062 | 17.291 | 10.081 | 1.00 | 23.63 |
| ATOM | 268 | OE1 | GLU | A | 570 | 3.218 | 17.158 | 9.632 | 1.00 | 21.68 |
| ATOM | 269 | OE2 | GLU | A | 570 | 1.047 | 17.311 | 9.352 | 1.00 | 28.09 |
| ATOM | 270 | C | GLU | A | 570 | 2.309 | 14.978 | 14.547 | 1.00 | 14.43 |
| ATOM | 271 | O | GLU | A | 570 | 1.293 | 14.319 | 14.780 | 1.00 | 15.20 |
| ATOM | 272 | N | THR | A | 571 | 3.529 | 14.551 | 14.866 | 1.00 | 12.67 |
| ATOM | 273 | CA | THR | A | 571 | 3.694 | 13.259 | 15.538 | 1.00 | 12.83 |
| ATOM | 274 | CB | THR | A | 571 | 5.182 | 12.816 | 15.638 | 1.00 | 13.30 |
| ATOM | 275 | OG1 | THR | A | 571 | 5.915 | 13.737 | 16.452 | 1.00 | 12.68 |
| ATOM | 276 | CG2 | THR | A | 571 | 5.817 | 12.738 | 14.250 | 1.00 | 12.38 |
| ATOM | 277 | C | THR | A | 571 | 3.110 | 13.360 | 16.945 | 1.00 | 11.29 |
| ATOM | 278 | O | THR | A | 571 | 2.589 | 12.381 | 17.483 | 1.00 | 12.14 |
| ATOM | 279 | N | ALA | A | 572 | 3.202 | 14.548 | 17.538 | 1.00 | 12.42 |
| ATOM | 280 | CA | ALA | A | 572 | 2.659 | 14.780 | 18.869 | 1.00 | 11.84 |
| ATOM | 281 | CB | ALA | A | 572 | 3.090 | 16.142 | 19.388 | 1.00 | 12.18 |
| ATOM | 282 | C | ALA | A | 572 | 1.136 | 14.701 | 18.803 | 1.00 | 13.34 |
| ATOM | 283 | O | ALA | A | 572 | 0.500 | 14.128 | 19.683 | 1.00 | 11.72 |
| ATOM | 284 | N | LEU | A | 573 | 0.554 | 15.283 | 17.758 | 1.00 | 12.67 |
| ATOM | 285 | CA | LEU | A | 573 | −0.894 | 15.244 | 17.583 | 1.00 | 13.98 |
| ATOM | 286 | CB | LEU | A | 573 | −1.307 | 16.104 | 16.384 | 1.00 | 14.33 |
| ATOM | 287 | CG | LEU | A | 573 | −1.135 | 17.619 | 16.544 | 1.00 | 14.55 |
| ATOM | 288 | CD1 | LEU | A | 573 | −1.445 | 18.310 | 15.224 | 1.00 | 21.57 |
| ATOM | 289 | CD2 | LEU | A | 573 | −2.061 | 18.125 | 17.647 | 1.00 | 20.27 |
| ATOM | 290 | C | LEU | A | 573 | −1.338 | 13.795 | 17.366 | 1.00 | 15.26 |
| ATOM | 291 | O | LEU | A | 573 | −2.394 | 13.378 | 17.853 | 1.00 | 15.01 |
| ATOM | 292 | N | CYS | A | 574 | −0.534 | 13.030 | 16.632 | 1.00 | 13.44 |
| ATOM | 293 | CA | CYS | A | 574 | −0.852 | 11.625 | 16.385 | 1.00 | 13.06 |
| ATOM | 294 | CB | CYS | A | 574 | 0.187 | 10.974 | 15.470 | 1.00 | 12.27 |
| ATOM | 295 | SG | CYS | A | 574 | 0.049 | 11.382 | 13.714 | 1.00 | 15.24 |
| ATOM | 296 | C | CYS | A | 574 | −0.873 | 10.864 | 17.703 | 1.00 | 12.15 |
| ATOM | 297 | O | CYS | A | 574 | −1.710 | 9.991 | 17.919 | 1.00 | 13.51 |
| ATOM | 298 | N | THR | A | 575 | 0.066 | 11.194 | 18.581 | 1.00 | 11.60 |
| ATOM | 299 | CA | THR | A | 575 | 0.158 | 10.532 | 19.872 | 1.00 | 12.28 |
| ATOM | 300 | CB | THR | A | 575 | 1.453 | 10.949 | 20.592 | 1.00 | 10.90 |
| ATOM | 301 | OG1 | THR | A | 575 | 2.575 | 10.576 | 19.777 | 1.00 | 13.47 |
| ATOM | 302 | CG2 | THR | A | 575 | 1.565 | 10.264 | 21.944 | 1.00 | 10.06 |
| ATOM | 303 | C | THR | A | 575 | −1.068 | 10.841 | 20.726 | 1.00 | 11.74 |
| ATOM | 304 | O | THR | A | 575 | −1.613 | 9.952 | 21.379 | 1.00 | 10.78 |
| ATOM | 305 | N | ILE | A | 576 | −1.514 | 12.095 | 20.717 | 1.00 | 12.29 |
| ATOM | 306 | CA | ILE | A | 576 | −2.700 | 12.453 | 21.484 | 1.00 | 11.92 |
| ATOM | 307 | CB | ILE | A | 576 | −3.046 | 13.945 | 21.354 | 1.00 | 11.38 |
| ATOM | 308 | CG2 | ILE | A | 576 | −4.371 | 14.222 | 22.053 | 1.00 | 12.83 |
| ATOM | 309 | CG1 | ILE | A | 576 | −1.933 | 14.802 | 21.965 | 1.00 | 17.73 |
| ATOM | 310 | CD1 | ILE | A | 576 | −2.154 | 16.292 | 21.798 | 1.00 | 13.34 |
| ATOM | 311 | C | ILE | A | 576 | −3.880 | 11.636 | 20.960 | 1.00 | 12.34 |
| ATOM | 312 | O | ILE | A | 576 | −4.674 | 11.103 | 21.737 | 1.00 | 13.64 |
| ATOM | 313 | N | ARG | A | 577 | −3.985 | 11.532 | 19.638 | 1.00 | 13.14 |
| ATOM | 314 | CA | ARG | A | 577 | −5.071 | 10.772 | 19.032 | 1.00 | 13.22 |
| ATOM | 315 | CB | ARG | A | 577 | −5.029 | 10.888 | 17.503 | 1.00 | 12.21 |
| ATOM | 316 | CG | ARG | A | 577 | −6.113 | 10.084 | 16.795 | 1.00 | 12.42 |
| ATOM | 317 | CD | ARG | A | 577 | −7.492 | 10.383 | 17.365 | 1.00 | 10.79 |
| ATOM | 318 | NE | ARG | A | 577 | −7.912 | 11.757 | 17.111 | 1.00 | 18.46 |
| ATOM | 319 | CZ | ARG | A | 577 | −9.015 | 12.304 | 17.612 | 1.00 | 16.47 |
| ATOM | 320 | NH1 | ARG | A | 577 | −9.319 | 13.564 | 17.325 | 1.00 | 24.31 |
| ATOM | 321 | NH2 | ARG | A | 577 | −9.809 | 11.596 | 18.405 | 1.00 | 18.21 |
| ATOM | 322 | C | ARG | A | 577 | −5.029 | 9.304 | 19.455 | 1.00 | 12.45 |
| ATOM | 323 | O | ARG | A | 577 | −6.069 | 8.675 | 19.625 | 1.00 | 11.98 |
| ATOM | 324 | N | MET | A | 578 | −3.831 | 8.756 | 19.630 | 1.00 | 10.43 |
| ATOM | 325 | CA | MET | A | 578 | −3.716 | 7.367 | 20.058 | 1.00 | 11.28 |
| ATOM | 326 | CB | MET | A | 578 | −2.245 | 6.934 | 20.090 | 1.00 | 11.91 |
| ATOM | 327 | CG | MET | A | 578 | −1.605 | 6.808 | 18.716 | 1.00 | 12.93 |
| ATOM | 328 | SD | MET | A | 578 | 0.134 | 6.324 | 18.830 | 1.00 | 16.07 |
| ATOM | 329 | CE | MET | A | 578 | 0.781 | 7.021 | 17.300 | 1.00 | 19.76 |
| ATOM | 330 | C | MET | A | 578 | −4.332 | 7.191 | 21.445 | 1.00 | 12.18 |
| ATOM | 331 | O | MET | A | 578 | −5.103 | 6.260 | 21.675 | 1.00 | 11.26 |
| ATOM | 332 | N | PHE | A | 579 | −3.982 | 8.080 | 22.372 | 1.00 | 11.60 |
| ATOM | 333 | CA | PHE | A | 579 | −4.521 | 8.004 | 23.725 | 1.00 | 11.60 |
| ATOM | 334 | CB | PHE | A | 579 | −3.921 | 9.097 | 24.619 | 1.00 | 12.17 |
| ATOM | 335 | CG | PHE | A | 579 | −2.584 | 8.748 | 25.201 | 1.00 | 9.68 |
| ATOM | 336 | CD1 | PHE | A | 579 | −1.415 | 9.023 | 24.510 | 1.00 | 13.42 |
| ATOM | 337 | CD2 | PHE | A | 579 | −2.497 | 8.148 | 26.450 | 1.00 | 12.49 |
| ATOM | 338 | CE1 | PHE | A | 579 | −0.178 | 8.709 | 25.054 | 1.00 | 10.87 |
| ATOM | 339 | CE2 | PHE | A | 579 | −1.264 | 7.829 | 27.000 | 1.00 | 13.01 |
| ATOM | 340 | CZ | PHE | A | 579 | −0.103 | 8.113 | 26.301 | 1.00 | 12.83 |
| ATOM | 341 | C | PHE | A | 579 | −6.040 | 8.170 | 23.708 | 1.00 | 12.84 |
| ATOM | 342 | O | PHE | A | 579 | −6.765 | 7.429 | 24.367 | 1.00 | 12.73 |
| ATOM | 343 | N | THR | A | 580 | −6.504 | 9.151 | 22.943 | 1.00 | 15.04 |
| ATOM | 344 | CA | THR | A | 580 | −7.926 | 9.459 | 22.839 | 1.00 | 14.30 |
| ATOM | 345 | CB | THR | A | 580 | −8.145 | 10.702 | 21.967 | 1.00 | 10.78 |
| ATOM | 346 | OG1 | THR | A | 580 | −7.344 | 11.780 | 22.469 | 1.00 | 18.08 |
| ATOM | 347 | CG2 | THR | A | 580 | −9.610 | 11.115 | 21.987 | 1.00 | 13.16 |
| ATOM | 348 | C | THR | A | 580 | −8.772 | 8.327 | 22.273 | 1.00 | 14.34 |
| ATOM | 349 | O | THR | A | 580 | −9.774 | 7.936 | 22.871 | 1.00 | 14.67 |
| ATOM | 350 | N | ASP | A | 581 | −8.367 | 7.806 | 21.119 | 1.00 | 15.75 |
| ATOM | 351 | CA | ASP | A | 581 | −9.106 | 6.731 | 20.466 | 1.00 | 14.61 |
| ATOM | 352 | CB | ASP | A | 581 | −8.724 | 6.668 | 18.990 | 1.00 | 16.27 |
| ATOM | 353 | CG | ASP | A | 581 | −9.398 | 7.757 | 18.181 | 1.00 | 20.38 |
| ATOM | 354 | OD1 | ASP | A | 581 | −9.762 | 8.797 | 18.776 | 1.00 | 15.70 |
| ATOM | 355 | OD2 | ASP | A | 581 | −9.560 | 7.580 | 16.958 | 1.00 | 17.57 |
| ATOM | 356 | C | ASP | A | 581 | −8.970 | 5.365 | 21.123 | 1.00 | 15.19 |
| ATOM | 357 | O | ASP | A | 581 | −9.627 | 4.402 | 20.719 | 1.00 | 14.62 |
| ATOM | 358 | N | LEU | A | 582 | −8.110 | 5.275 | 22.130 | 1.00 | 13.55 |
| ATOM | 359 | CA | LEU | A | 582 | −7.957 | 4.032 | 22.867 | 1.00 | 14.36 |
| ATOM | 360 | CB | LEU | A | 582 | −6.484 | 3.739 | 23.156 | 1.00 | 16.24 |
| ATOM | 361 | CG | LEU | A | 582 | −5.770 | 3.024 | 22.006 | 1.00 | 14.56 |
| ATOM | 362 | CD1 | LEU | A | 582 | −4.272 | 2.968 | 22.250 | 1.00 | 12.82 |
| ATOM | 363 | CD2 | LEU | A | 582 | −6.351 | 1.625 | 21.865 | 1.00 | 12.05 |
| ATOM | 364 | C | LEU | A | 582 | −8.739 | 4.189 | 24.168 | 1.00 | 17.31 |
| ATOM | 365 | O | LEU | A | 582 | −8.617 | 3.378 | 25.081 | 1.00 | 17.82 |
| ATOM | 366 | N | ASN | A | 583 | −9.533 | 5.255 | 24.246 | 1.00 | 17.93 |
| ATOM | 367 | CA | ASN | A | 583 | −10.361 | 5.500 | 25.420 | 1.00 | 21.01 |
| ATOM | 368 | CB | ASN | A | 583 | −11.405 | 4.377 | 25.525 | 1.00 | 22.07 |
| ATOM | 369 | CG | ASN | A | 583 | −12.368 | 4.564 | 26.682 | 1.00 | 34.95 |
| ATOM | 370 | OD1 | ASN | A | 583 | −12.921 | 5.645 | 26.879 | 1.00 | 34.93 |
| ATOM | 371 | ND2 | ASN | A | 583 | −12.589 | 3.496 | 27.444 | 1.00 | 39.94 |
| ATOM | 372 | C | ASN | A | 583 | −9.516 | 5.575 | 26.693 | 1.00 | 23.16 |
| ATOM | 373 | O | ASN | A | 583 | −9.866 | 4.978 | 27.713 | 1.00 | 27.41 |
| ATOM | 374 | N | LEU | A | 584 | −8.406 | 6.313 | 26.634 | 1.00 | 18.24 |
| ATOM | 375 | CA | LEU | A | 584 | −7.517 | 6.446 | 27.791 | 1.00 | 18.39 |
| ATOM | 376 | CB | LEU | A | 584 | −6.060 | 6.188 | 27.379 | 1.00 | 13.74 |
| ATOM | 377 | CG | LEU | A | 584 | −5.713 | 4.823 | 26.778 | 1.00 | 13.61 |
| ATOM | 378 | CD1 | LEU | A | 584 | −4.226 | 4.789 | 26.412 | 1.00 | 16.18 |
| ATOM | 379 | CD2 | LEU | A | 584 | −6.050 | 3.726 | 27.769 | 1.00 | 15.78 |
| ATOM | 380 | C | LEU | A | 584 | −7.591 | 7.807 | 28.486 | 1.00 | 18.85 |
| ATOM | 381 | O | LEU | A | 584 | −7.377 | 7.904 | 29.695 | 1.00 | 18.54 |
| ATOM | 382 | N | VAL | A | 585 | −7.890 | 8.853 | 27.721 | 1.00 | 18.53 |
| ATOM | 383 | CA | VAL | A | 585 | −7.956 | 10.211 | 28.258 | 1.00 | 20.90 |
| ATOM | 384 | CB | VAL | A | 585 | −8.157 | 11.234 | 27.123 | 1.00 | 25.63 |
| ATOM | 385 | CG1 | VAL | A | 585 | −8.083 | 12.648 | 27.672 | 1.00 | 26.57 |
| ATOM | 386 | CG2 | VAL | A | 585 | −7.102 | 11.024 | 26.054 | 1.00 | 26.78 |
| ATOM | 387 | C | VAL | A | 585 | −9.049 | 10.416 | 29.307 | 1.00 | 21.72 |
| ATOM | 388 | O | VAL | A | 585 | −8.771 | 10.839 | 30.429 | 1.00 | 21.94 |
| ATOM | 389 | N | GLN | A | 586 | −10.291 | 10.118 | 28.945 | 1.00 | 22.79 |
| ATOM | 390 | CA | GLN | A | 586 | −11.394 | 10.290 | 29.881 | 1.00 | 25.11 |
| ATOM | 391 | CB | GLN | A | 586 | −12.728 | 10.306 | 29.131 | 1.00 | 34.29 |
| ATOM | 392 | CG | GLN | A | 586 | −13.719 | 11.317 | 29.678 | 1.00 | 39.65 |
| ATOM | 393 | CD | GLN | A | 586 | −13.155 | 12.725 | 29.663 | 1.00 | 50.17 |
| ATOM | 394 | OE1 | GLN | A | 586 | −12.715 | 13.218 | 28.622 | 1.00 | 52.20 |
| ATOM | 395 | NE2 | GLN | A | 586 | −13.160 | 13.380 | 30.820 | 1.00 | 48.68 |
| ATOM | 396 | C | GLN | A | 586 | −11.402 | 9.172 | 30.914 | 1.00 | 22.36 |
| ATOM | 397 | O | GLN | A | 586 | −11.587 | 9.413 | 32.108 | 1.00 | 23.81 |
| ATOM | 398 | N | ASN | A | 587 | −11.195 | 7.948 | 30.441 | 1.00 | 22.47 |
| ATOM | 399 | CA | ASN | A | 587 | −11.181 | 6.769 | 31.298 | 1.00 | 19.07 |
| ATOM | 400 | CB | ASN | A | 587 | −10.651 | 5.565 | 30.507 | 1.00 | 21.06 |
| ATOM | 401 | CG | ASN | A | 587 | −10.944 | 4.236 | 31.179 | 1.00 | 29.14 |
| ATOM | 402 | OD1 | ASN | A | 587 | −11.098 | 4.157 | 32.397 | 1.00 | 21.26 |
| ATOM | 403 | ND2 | ASN | A | 587 | −11.002 | 3.174 | 30.382 | 1.00 | 33.44 |
| ATOM | 404 | C | ASN | A | 587 | −10.319 | 6.981 | 32.542 | 1.00 | 20.86 |
| ATOM | 405 | O | ASN | A | 587 | −10.727 | 6.646 | 33.656 | 1.00 | 19.43 |
| ATOM | 406 | N | PHE | A | 588 | −9.134 | 7.553 | 32.357 | 1.00 | 18.57 |
| ATOM | 407 | CA | PHE | A | 588 | −8.227 | 7.753 | 33.480 | 1.00 | 18.65 |
| ATOM | 408 | CB | PHE | A | 588 | −6.880 | 7.109 | 33.145 | 1.00 | 13.88 |
| ATOM | 409 | CG | PHE | A | 588 | −6.976 | 5.624 | 32.948 | 1.00 | 14.75 |
| ATOM | 410 | CD1 | PHE | A | 588 | −7.272 | 4.791 | 34.022 | 1.00 | 13.58 |
| ATOM | 411 | CD2 | PHE | A | 588 | −6.860 | 5.065 | 31.686 | 1.00 | 12.05 |
| ATOM | 412 | CE1 | PHE | A | 588 | −7.456 | 3.431 | 33.837 | 1.00 | 13.16 |
| ATOM | 413 | CE2 | PHE | A | 588 | −7.044 | 3.704 | 31.496 | 1.00 | 10.45 |
| ATOM | 414 | CZ | PHE | A | 588 | −7.344 | 2.888 | 32.576 | 1.00 | 16.21 |
| ATOM | 415 | C | PHE | A | 588 | −8.063 | 9.190 | 33.966 | 1.00 | 20.57 |
| ATOM | 416 | O | PHE | A | 588 | −7.072 | 9.538 | 34.611 | 1.00 | 18.90 |
| ATOM | 417 | N | GLN | A | 589 | −9.052 | 10.020 | 33.662 | 1.00 | 21.41 |
| ATOM | 418 | CA | GLN | A | 589 | −9.057 | 11.410 | 34.110 | 1.00 | 23.36 |
| ATOM | 419 | CB | GLN | A | 589 | −9.213 | 11.449 | 35.634 | 1.00 | 28.73 |
| ATOM | 420 | CG | GLN | A | 589 | −10.311 | 10.551 | 36.194 | 1.00 | 40.43 |
| ATOM | 421 | CD | GLN | A | 589 | −11.701 | 11.003 | 35.797 | 1.00 | 42.17 |
| ATOM | 422 | OE1 | GLN | A | 589 | −12.058 | 12.167 | 35.971 | 1.00 | 38.12 |
| ATOM | 423 | NE2 | GLN | A | 589 | −12.498 | 10.080 | 35.270 | 1.00 | 47.35 |
| ATOM | 424 | C | GLN | A | 589 | −7.805 | 12.201 | 33.725 | 1.00 | 20.90 |
| ATOM | 425 | O | GLN | A | 589 | −7.282 | 12.972 | 34.531 | 1.00 | 21.16 |
| ATOM | 426 | N | MET | A | 590 | −7.321 | 12.019 | 32.502 | 1.00 | 22.12 |
| ATOM | 427 | CA | MET | A | 590 | −6.133 | 12.745 | 32.069 | 1.00 | 21.73 |
| ATOM | 428 | CB | MET | A | 590 | −5.463 | 12.035 | 30.893 | 1.00 | 22.93 |
| ATOM | 429 | CG | MET | A | 590 | −4.751 | 10.752 | 31.257 | 1.00 | 24.06 |
| ATOM | 430 | SD | MET | A | 590 | −3.865 | 10.087 | 29.838 | 1.00 | 19.72 |
| ATOM | 431 | CE | MET | A | 590 | −3.631 | 8.404 | 30.357 | 1.00 | 17.35 |
| ATOM | 432 | C | MET | A | 590 | −6.455 | 14.172 | 31.654 | 1.00 | 22.68 |
| ATOM | 433 | O | MET | A | 590 | −7.295 | 14.398 | 30.784 | 1.00 | 23.69 |
| ATOM | 434 | N | LYS | A | 591 | −5.792 | 15.138 | 32.278 | 1.00 | 20.35 |
| ATOM | 435 | CA | LYS | A | 591 | −6.013 | 16.530 | 31.919 | 1.00 | 20.85 |
| ATOM | 436 | CB | LYS | A | 591 | −5.554 | 17.460 | 33.045 | 1.00 | 22.95 |
| ATOM | 437 | CG | LYS | A | 591 | −6.472 | 17.422 | 34.262 | 1.00 | 28.61 |
| ATOM | 438 | CD | LYS | A | 591 | −6.065 | 18.438 | 35.315 | 1.00 | 37.59 |
| ATOM | 439 | CE | LYS | A | 591 | −7.106 | 18.527 | 36.423 | 1.00 | 42.92 |
| ATOM | 440 | NZ | LYS | A | 591 | −8.417 | 19.026 | 35.915 | 1.00 | 51.43 |
| ATOM | 441 | C | LYS | A | 591 | −5.235 | 16.800 | 30.636 | 1.00 | 20.72 |
| ATOM | 442 | O | LYS | A | 591 | −4.048 | 16.486 | 30.537 | 1.00 | 21.29 |
| ATOM | 443 | N | HIS | A | 592 | −5.920 | 17.375 | 29.654 | 1.00 | 20.84 |
| ATOM | 444 | CA | HIS | A | 592 | −5.333 | 17.670 | 28.351 | 1.00 | 21.11 |
| ATOM | 445 | CB | HIS | A | 592 | −6.273 | 18.577 | 27.553 | 1.00 | 21.39 |
| ATOM | 446 | CG | HIS | A | 592 | −5.884 | 18.737 | 26.125 | 1.00 | 18.05 |
| ATOM | 447 | CD2 | HIS | A | 592 | −5.427 | 19.798 | 25.424 | 1.00 | 24.75 |
| ATOM | 448 | ND1 | HIS | A | 592 | −5.926 | 17.685 | 25.213 | 1.00 | 22.74 |
| ATOM | 449 | CE1 | HIS | A | 592 | −5.518 | 18.102 | 24.044 | 1.00 | 29.20 |
| ATOM | 450 | NE2 | HIS | A | 592 | −5.204 | 19.393 | 24.135 | 1.00 | 27.94 |
| ATOM | 451 | C | HIS | A | 592 | −3.937 | 18.295 | 28.377 | 1.00 | 20.49 |
| ATOM | 452 | O | HIS | A | 592 | −3.034 | 17.848 | 27.661 | 1.00 | 19.49 |
| ATOM | 453 | N | GLU | A | 593 | −3.765 | 19.338 | 29.184 | 1.00 | 19.12 |
| ATOM | 454 | CA | GLU | A | 593 | −2.481 | 20.019 | 29.279 | 1.00 | 18.26 |
| ATOM | 455 | CB | GLU | A | 593 | −2.592 | 21.243 | 30.192 | 1.00 | 23.02 |
| ATOM | 456 | CG | GLU | A | 593 | −1.267 | 21.957 | 30.416 | 1.00 | 32.52 |
| ATOM | 457 | CD | GLU | A | 593 | −1.422 | 23.296 | 31.116 | 1.00 | 44.01 |
| ATOM | 458 | OE1 | GLU | A | 593 | −2.062 | 24.200 | 30.535 | 1.00 | 41.08 |
| ATOM | 459 | OE2 | GLU | A | 593 | −0.901 | 23.446 | 32.244 | 1.00 | 45.85 |
| ATOM | 460 | C | GLU | A | 593 | −1.381 | 19.099 | 29.793 | 1.00 | 18.42 |
| ATOM | 461 | O | GLU | A | 593 | −0.230 | 19.195 | 29.365 | 1.00 | 16.87 |
| ATOM | 462 | N | VAL | A | 594 | −1.738 | 18.209 | 30.710 | 1.00 | 18.22 |
| ATOM | 463 | CA | VAL | A | 594 | −0.770 | 17.275 | 31.277 | 1.00 | 17.13 |
| ATOM | 464 | CB | VAL | A | 594 | −1.372 | 16.506 | 32.465 | 1.00 | 16.16 |
| ATOM | 465 | CG1 | VAL | A | 594 | −0.314 | 15.605 | 33.085 | 1.00 | 20.37 |
| ATOM | 466 | CG2 | VAL | A | 594 | −1.911 | 17.481 | 33.491 | 1.00 | 22.54 |
| ATOM | 467 | C | VAL | A | 594 | −0.299 | 16.263 | 30.235 | 1.00 | 15.25 |
| ATOM | 468 | O | VAL | A | 594 | 0.902 | 16.005 | 30.101 | 1.00 | 16.26 |
| ATOM | 469 | N | LEU | A | 595 | −1.246 | 15.681 | 29.504 | 1.00 | 13.83 |
| ATOM | 470 | CA | LEU | A | 595 | −0.897 | 14.705 | 28.478 | 1.00 | 14.01 |
| ATOM | 471 | CB | LEU | A | 595 | −2.163 | 14.147 | 27.818 | 1.00 | 9.50 |
| ATOM | 472 | CG | LEU | A | 595 | −1.969 | 13.167 | 26.653 | 1.00 | 18.45 |
| ATOM | 473 | CD1 | LEU | A | 595 | −1.068 | 12.014 | 27.076 | 1.00 | 12.38 |
| ATOM | 474 | CD2 | LEU | A | 595 | −3.318 | 12.643 | 26.201 | 1.00 | 14.12 |
| ATOM | 475 | C | LEU | A | 595 | −0.002 | 15.372 | 27.438 | 1.00 | 13.61 |
| ATOM | 476 | O | LEU | A | 595 | 1.004 | 14.804 | 27.010 | 1.00 | 13.98 |
| ATOM | 477 | N | CYS | A | 596 | −0.358 | 16.588 | 27.037 | 1.00 | 14.70 |
| ATOM | 478 | CA | CYS | A | 596 | 0.448 | 17.303 | 26.058 | 1.00 | 12.24 |
| ATOM | 479 | CB | CYS | A | 596 | −0.191 | 18.649 | 25.709 | 1.00 | 15.21 |
| ATOM | 480 | SG | CYS | A | 596 | −1.702 | 18.507 | 24.749 | 1.00 | 17.45 |
| ATOM | 481 | C | CYS | A | 596 | 1.861 | 17.534 | 26.586 | 1.00 | 12.70 |
| ATOM | 482 | O | CYS | A | 596 | 2.839 | 17.327 | 25.868 | 1.00 | 11.06 |
| ATOM | 483 | N | ARG | A | 597 | 1.970 | 17.959 | 27.841 | 1.00 | 11.36 |
| ATOM | 484 | CA | ARG | A | 597 | 3.283 | 18.211 | 28.420 | 1.00 | 11.51 |
| ATOM | 485 | CB | ARG | A | 597 | 3.141 | 18.836 | 29.815 | 1.00 | 14.74 |
| ATOM | 486 | CG | ARG | A | 597 | 4.463 | 19.343 | 30.388 | 1.00 | 15.86 |
| ATOM | 487 | CD | ARG | A | 597 | 4.294 | 20.067 | 31.727 | 1.00 | 20.22 |
| ATOM | 488 | NE | ARG | A | 597 | 3.565 | 21.332 | 31.614 | 1.00 | 21.93 |
| ATOM | 489 | CZ | ARG | A | 597 | 2.279 | 21.493 | 31.916 | 1.00 | 27.00 |
| ATOM | 490 | NH1 | ARG | A | 597 | 1.560 | 20.469 | 32.357 | 1.00 | 22.77 |
| ATOM | 491 | NH2 | ARG | A | 597 | 1.708 | 22.683 | 31.782 | 1.00 | 30.27 |
| ATOM | 492 | C | ARG | A | 597 | 4.100 | 16.919 | 28.494 | 1.00 | 11.80 |
| ATOM | 493 | O | ARG | A | 597 | 5.287 | 16.909 | 28.162 | 1.00 | 10.48 |
| ATOM | 494 | N | TRP | A | 598 | 3.454 | 15.830 | 28.905 | 1.00 | 11.97 |
| ATOM | 495 | CA | TRP | A | 598 | 4.112 | 14.530 | 29.022 | 1.00 | 9.62 |
| ATOM | 496 | CB | TRP | A | 598 | 3.125 | 13.488 | 29.556 | 1.00 | 11.57 |
| ATOM | 497 | CG | TRP | A | 598 | 3.717 | 12.110 | 29.702 | 1.00 | 10.65 |
| ATOM | 498 | CD2 | TRP | A | 598 | 3.609 | 11.028 | 28.771 | 1.00 | 9.66 |
| ATOM | 499 | CE2 | TRP | A | 598 | 4.350 | 9.948 | 29.298 | 1.00 | 8.24 |
| ATOM | 500 | CE3 | TRP | A | 598 | 2.964 | 10.866 | 27.541 | 1.00 | 11.87 |
| ATOM | 501 | CD1 | TRP | A | 598 | 4.494 | 11.656 | 30.728 | 1.00 | 9.74 |
| ATOM | 502 | NE1 | TRP | A | 598 | 4.878 | 10.359 | 30.492 | 1.00 | 11.42 |
| ATOM | 503 | CZ2 | TRP | A | 598 | 4.459 | 8.720 | 28.635 | 1.00 | 7.38 |
| ATOM | 504 | CZ3 | TRP | A | 598 | 3.075 | 9.646 | 26.886 | 1.00 | 9.43 |
| ATOM | 505 | CH2 | TRP | A | 598 | 3.818 | 8.593 | 27.434 | 1.00 | 10.02 |
| ATOM | 506 | C | TRP | A | 598 | 4.656 | 14.064 | 27.670 | 1.00 | 12.66 |
| ATOM | 507 | O | TRP | A | 598 | 5.804 | 13.617 | 27.568 | 1.00 | 11.33 |
| ATOM | 508 | N | ILE | A | 599 | 3.823 | 14.160 | 26.637 | 1.00 | 9.84 |
| ATOM | 509 | CA | ILE | A | 599 | 4.232 | 13.748 | 25.298 | 1.00 | 12.03 |
| ATOM | 510 | CB | ILE | A | 599 | 3.101 | 13.974 | 24.270 | 1.00 | 11.30 |
| ATOM | 511 | CG2 | ILE | A | 599 | 3.648 | 13.807 | 22.856 | 1.00 | 13.41 |
| ATOM | 512 | CG1 | ILE | A | 599 | 1.953 | 13.000 | 24.546 | 1.00 | 13.16 |
| ATOM | 513 | CD1 | ILE | A | 599 | 0.732 | 13.218 | 23.668 | 1.00 | 13.06 |
| ATOM | 514 | C | ILE | A | 599 | 5.460 | 14.533 | 24.855 | 1.00 | 14.21 |
| ATOM | 515 | O | ILE | A | 599 | 6.414 | 13.969 | 24.308 | 1.00 | 11.47 |
| ATOM | 516 | N | LEU | A | 600 | 5.439 | 15.839 | 25.094 | 1.00 | 14.33 |
| ATOM | 517 | CA | LEU | A | 600 | 6.566 | 16.670 | 24.707 | 1.00 | 13.61 |
| ATOM | 518 | CB | LEU | A | 600 | 6.214 | 18.151 | 24.882 | 1.00 | 13.53 |
| ATOM | 519 | CG | LEU | A | 600 | 5.073 | 18.621 | 23.971 | 1.00 | 12.80 |
| ATOM | 520 | CD1 | LEU | A | 600 | 4.652 | 20.036 | 24.336 | 1.00 | 15.66 |
| ATOM | 521 | CD2 | LEU | A | 600 | 5.518 | 18.557 | 22.520 | 1.00 | 13.09 |
| ATOM | 522 | C | LEU | A | 600 | 7.800 | 16.297 | 25.523 | 1.00 | 10.78 |
| ATOM | 523 | O | LEU | A | 600 | 8.917 | 16.324 | 25.011 | 1.00 | 10.47 |
| ATOM | 524 | N | SER | A | 601 | 7.601 | 15.925 | 26.788 | 1.00 | 10.50 |
| ATOM | 525 | CA | SER | A | 601 | 8.734 | 15.540 | 27.624 | 1.00 | 8.54 |
| ATOM | 526 | CB | SER | A | 601 | 8.303 | 15.398 | 29.085 | 1.00 | 9.00 |
| ATOM | 527 | OG | SER | A | 601 | 8.036 | 16.678 | 29.634 | 1.00 | 9.87 |
| ATOM | 528 | C | SER | A | 601 | 9.342 | 14.239 | 27.129 | 1.00 | 9.99 |
| ATOM | 529 | O | SER | A | 601 | 10.563 | 14.082 | 27.104 | 1.00 | 10.43 |
| ATOM | 530 | N | VAL | A | 602 | 8.487 | 13.300 | 26.739 | 1.00 | 10.77 |
| ATOM | 531 | CA | VAL | A | 602 | 8.968 | 12.024 | 26.223 | 1.00 | 10.98 |
| ATOM | 532 | CB | VAL | A | 602 | 7.791 | 11.081 | 25.874 | 1.00 | 12.52 |
| ATOM | 533 | CG1 | VAL | A | 602 | 8.303 | 9.856 | 25.138 | 1.00 | 9.22 |
| ATOM | 534 | CG2 | VAL | A | 602 | 7.068 | 10.658 | 27.152 | 1.00 | 11.71 |
| ATOM | 535 | C | VAL | A | 602 | 9.794 | 12.290 | 24.967 | 1.00 | 12.57 |
| ATOM | 536 | O | VAL | A | 602 | 10.935 | 11.848 | 24.855 | 1.00 | 11.99 |
| ATOM | 537 | N | LYS | A | 603 | 9.220 | 13.043 | 24.034 | 1.00 | 12.97 |
| ATOM | 538 | CA | LYS | A | 603 | 9.906 | 13.363 | 22.792 | 1.00 | 11.73 |
| ATOM | 539 | CB | LYS | A | 603 | 9.025 | 14.268 | 21.928 | 1.00 | 14.00 |
| ATOM | 540 | CG | LYS | A | 603 | 9.666 | 14.688 | 20.620 | 1.00 | 14.56 |
| ATOM | 541 | CD | LYS | A | 603 | 8.656 | 15.317 | 19.686 | 1.00 | 16.82 |
| ATOM | 542 | CE | LYS | A | 603 | 8.088 | 16.603 | 20.262 | 1.00 | 24.97 |
| ATOM | 543 | NZ | LYS | A | 603 | 7.169 | 17.256 | 19.300 | 1.00 | 39.23 |
| ATOM | 544 | C | LYS | A | 603 | 11.256 | 14.035 | 23.042 | 1.00 | 11.50 |
| ATOM | 545 | O | LYS | A | 603 | 12.252 | 13.694 | 22.414 | 1.00 | 14.04 |
| ATOM | 546 | N | LYS | A | 604 | 11.279 | 14.985 | 23.968 | 1.00 | 11.12 |
| ATOM | 547 | CA | LYS | A | 604 | 12.501 | 15.714 | 24.293 | 1.00 | 13.84 |
| ATOM | 548 | CB | LYS | A | 604 | 12.181 | 16.849 | 25.274 | 1.00 | 16.59 |
| ATOM | 549 | CG | LYS | A | 604 | 13.403 | 17.589 | 25.788 | 1.00 | 18.98 |
| ATOM | 550 | CD | LYS | A | 604 | 13.016 | 18.673 | 26.784 | 1.00 | 25.53 |
| ATOM | 551 | CE | LYS | A | 604 | 14.251 | 19.335 | 27.391 | 1.00 | 31.51 |
| ATOM | 552 | NZ | LYS | A | 604 | 13.893 | 20.351 | 28.424 | 1.00 | 29.80 |
| ATOM | 553 | C | LYS | A | 604 | 13.582 | 14.824 | 24.890 | 1.00 | 12.98 |
| ATOM | 554 | O | LYS | A | 604 | 14.776 | 15.073 | 24.710 | 1.00 | 13.10 |
| ATOM | 555 | N | ASN | A | 605 | 13.173 | 13.773 | 25.587 | 1.00 | 12.62 |
| ATOM | 556 | CA | ASN | A | 605 | 14.146 | 12.901 | 26.220 | 1.00 | 13.04 |
| ATOM | 557 | CB | ASN | A | 605 | 13.555 | 12.336 | 27.507 | 1.00 | 13.13 |
| ATOM | 558 | CG | ASN | A | 605 | 13.579 | 13.356 | 28.638 | 1.00 | 32.58 |
| ATOM | 559 | OD1 | ASN | A | 605 | 14.638 | 13.650 | 29.197 | 1.00 | 25.76 |
| ATOM | 560 | ND2 | ASN | A | 605 | 12.418 | 13.918 | 28.962 | 1.00 | 30.95 |
| ATOM | 561 | C | ASN | A | 605 | 14.757 | 11.809 | 25.352 | 1.00 | 15.12 |
| ATOM | 562 | O | ASN | A | 605 | 15.342 | 10.848 | 25.858 | 1.00 | 18.48 |
| ATOM | 563 | N | TYR | A | 606 | 14.612 | 11.961 | 24.042 | 1.00 | 10.60 |
| ATOM | 564 | CA | TYR | A | 606 | 15.233 | 11.051 | 23.093 | 1.00 | 10.45 |
| ATOM | 565 | CB | TYR | A | 606 | 14.254 | 10.645 | 21.982 | 1.00 | 10.34 |
| ATOM | 566 | CG | TYR | A | 606 | 13.420 | 9.444 | 22.358 | 1.00 | 11.02 |
| ATOM | 567 | CD1 | TYR | A | 606 | 13.950 | 8.156 | 22.297 | 1.00 | 8.31 |
| ATOM | 568 | CE1 | TYR | A | 606 | 13.222 | 7.061 | 22.752 | 1.00 | 8.28 |
| ATOM | 569 | CD2 | TYR | A | 606 | 12.139 | 9.601 | 22.874 | 1.00 | 12.47 |
| ATOM | 570 | CE2 | TYR | A | 606 | 11.410 | 8.516 | 23.335 | 1.00 | 15.05 |
| ATOM | 571 | CZ | TYR | A | 606 | 11.955 | 7.252 | 23.275 | 1.00 | 15.46 |
| ATOM | 572 | OH | TYR | A | 606 | 11.227 | 6.188 | 23.773 | 1.00 | 12.36 |
| ATOM | 573 | C | TYR | A | 606 | 16.362 | 11.908 | 22.539 | 1.00 | 11.41 |
| ATOM | 574 | O | TYR | A | 606 | 16.216 | 13.129 | 22.428 | 1.00 | 14.91 |
| ATOM | 575 | N | ARG | A | 607 | 17.499 | 11.293 | 22.234 | 1.00 | 11.41 |
| ATOM | 576 | CA | ARG | A | 607 | 18.627 | 12.042 | 21.700 | 1.00 | 10.90 |
| ATOM | 577 | CB | ARG | A | 607 | 19.933 | 11.322 | 22.034 | 1.00 | 12.68 |
| ATOM | 578 | CG | ARG | A | 607 | 20.078 | 11.019 | 23.520 | 1.00 | 10.36 |
| ATOM | 579 | CD | ARG | A | 607 | 21.460 | 10.489 | 23.870 | 1.00 | 13.25 |
| ATOM | 580 | NE | ARG | A | 607 | 21.492 | 9.929 | 25.221 | 1.00 | 9.55 |
| ATOM | 581 | CZ | ARG | A | 607 | 22.601 | 9.556 | 25.854 | 1.00 | 18.33 |
| ATOM | 582 | NH1 | ARG | A | 607 | 23.781 | 9.686 | 25.262 | 1.00 | 13.50 |
| ATOM | 583 | NH2 | ARG | A | 607 | 22.530 | 9.045 | 27.077 | 1.00 | 17.68 |
| ATOM | 584 | C | ARG | A | 607 | 18.425 | 12.150 | 20.194 | 1.00 | 10.45 |
| ATOM | 585 | O | ARG | A | 607 | 18.616 | 11.182 | 19.458 | 1.00 | 12.53 |
| ATOM | 586 | N | LYS | A | 608 | 18.052 | 13.340 | 19.738 | 1.00 | 11.88 |
| ATOM | 587 | CA | LYS | A | 608 | 17.755 | 13.555 | 18.323 | 1.00 | 13.33 |
| ATOM | 588 | CB | LYS | A | 608 | 17.273 | 14.993 | 18.104 | 1.00 | 18.33 |
| ATOM | 589 | CG | LYS | A | 608 | 18.226 | 16.064 | 18.574 | 1.00 | 34.41 |
| ATOM | 590 | CD | LYS | A | 608 | 17.558 | 17.429 | 18.538 | 1.00 | 39.16 |
| ATOM | 591 | CE | LYS | A | 608 | 18.430 | 18.485 | 19.193 | 1.00 | 44.56 |
| ATOM | 592 | NZ | LYS | A | 608 | 19.755 | 18.595 | 18.525 | 1.00 | 49.34 |
| ATOM | 593 | C | LYS | A | 608 | 18.837 | 13.220 | 17.311 | 1.00 | 13.41 |
| ATOM | 594 | O | LYS | A | 608 | 18.527 | 12.904 | 16.164 | 1.00 | 14.71 |
| ATOM | 595 | N | ASN | A | 609 | 20.098 | 13.272 | 17.724 | 1.00 | 12.65 |
| ATOM | 596 | CA | ASN | A | 609 | 21.185 | 12.977 | 16.804 | 1.00 | 15.37 |
| ATOM | 597 | CB | ASN | A | 609 | 22.383 | 13.872 | 17.121 | 1.00 | 19.55 |
| ATOM | 598 | CG | ASN | A | 609 | 22.059 | 15.347 | 16.952 | 1.00 | 21.29 |
| ATOM | 599 | OD1 | ASN | A | 609 | 21.630 | 15.779 | 15.882 | 1.00 | 28.72 |
| ATOM | 600 | ND2 | ASN | A | 609 | 22.261 | 16.126 | 18.009 | 1.00 | 36.76 |
| ATOM | 601 | C | ASN | A | 609 | 21.593 | 11.507 | 16.743 | 1.00 | 14.47 |
| ATOM | 602 | O | ASN | A | 609 | 22.486 | 11.135 | 15.981 | 1.00 | 15.29 |
| ATOM | 603 | N | VAL | A | 610 | 20.955 | 10.670 | 17.557 | 1.00 | 11.89 |
| ATOM | 604 | CA | VAL | A | 610 | 21.221 | 9.233 | 17.511 | 1.00 | 11.43 |
| ATOM | 605 | CB | VAL | A | 610 | 20.696 | 8.531 | 18.777 | 1.00 | 11.38 |
| ATOM | 606 | CG1 | VAL | A | 610 | 20.649 | 7.019 | 18.571 | 1.00 | 10.56 |
| ATOM | 607 | CG2 | VAL | A | 610 | 21.608 | 8.882 | 19.955 | 1.00 | 10.47 |
| ATOM | 608 | C | VAL | A | 610 | 20.432 | 8.809 | 16.272 | 1.00 | 12.97 |
| ATOM | 609 | O | VAL | A | 610 | 19.221 | 9.025 | 16.198 | 1.00 | 11.59 |
| ATOM | 610 | N | ALA | A | 611 | 21.131 | 8.223 | 15.302 | 1.00 | 11.37 |
| ATOM | 611 | CA | ALA | A | 611 | 20.554 | 7.833 | 14.018 | 1.00 | 12.77 |
| ATOM | 612 | CB | ALA | A | 611 | 21.579 | 7.021 | 13.223 | 1.00 | 12.83 |
| ATOM | 613 | C | ALA | A | 611 | 19.208 | 7.117 | 13.981 | 1.00 | 12.88 |
| ATOM | 614 | O | ALA | A | 611 | 18.305 | 7.522 | 13.247 | 1.00 | 14.79 |
| ATOM | 615 | N | TYR | A | 612 | 19.071 | 6.050 | 14.756 | 1.00 | 12.18 |
| ATOM | 616 | CA | TYR | A | 612 | 17.830 | 5.288 | 14.743 | 1.00 | 10.69 |
| ATOM | 617 | CB | TYR | A | 612 | 18.147 | 3.824 | 14.408 | 1.00 | 13.15 |
| ATOM | 618 | CG | TYR | A | 612 | 16.968 | 2.877 | 14.509 | 1.00 | 12.63 |
| ATOM | 619 | CD1 | TYR | A | 612 | 15.832 | 3.054 | 13.729 | 1.00 | 19.45 |
| ATOM | 620 | CE1 | TYR | A | 612 | 14.752 | 2.185 | 13.833 | 1.00 | 24.00 |
| ATOM | 621 | CD2 | TYR | A | 612 | 16.996 | 1.805 | 15.392 | 1.00 | 13.47 |
| ATOM | 622 | CE2 | TYR | A | 612 | 15.930 | 0.938 | 15.503 | 1.00 | 24.95 |
| ATOM | 623 | CZ | TYR | A | 612 | 14.812 | 1.129 | 14.722 | 1.00 | 24.27 |
| ATOM | 624 | OH | TYR | A | 612 | 13.761 | 0.245 | 14.831 | 1.00 | 25.94 |
| ATOM | 625 | C | TYR | A | 612 | 17.017 | 5.371 | 16.028 | 1.00 | 11.05 |
| ATOM | 626 | O | TYR | A | 612 | 15.812 | 5.632 | 15.989 | 1.00 | 9.25 |
| ATOM | 627 | N | HIS | A | 613 | 17.667 | 5.159 | 17.166 | 1.00 | 10.71 |
| ATOM | 628 | CA | HIS | A | 613 | 16.951 | 5.199 | 18.432 | 1.00 | 9.46 |
| ATOM | 629 | CB | HIS | A | 613 | 17.694 | 4.391 | 19.499 | 1.00 | 12.29 |
| ATOM | 630 | CG | HIS | A | 613 | 17.706 | 2.923 | 19.232 | 1.00 | 13.22 |
| ATOM | 631 | CD2 | HIS | A | 613 | 16.742 | 1.983 | 19.364 | 1.00 | 13.53 |
| ATOM | 632 | ND1 | HIS | A | 613 | 18.811 | 2.262 | 18.726 | 1.00 | 8.83 |
| ATOM | 633 | CE1 | HIS | A | 613 | 18.523 | 0.989 | 18.562 | 1.00 | 12.86 |
| ATOM | 634 | NE2 | HIS | A | 613 | 17.268 | 0.789 | 18.942 | 1.00 | 16.65 |
| ATOM | 635 | C | HIS | A | 613 | 16.686 | 6.606 | 18.936 | 1.00 | 11.81 |
| ATOM | 636 | O | HIS | A | 613 | 17.226 | 7.023 | 19.957 | 1.00 | 11.26 |
| ATOM | 637 | N | ASN | A | 614 | 15.859 | 7.333 | 18.192 | 1.00 | 12.20 |
| ATOM | 638 | CA | ASN | A | 614 | 15.459 | 8.685 | 18.556 | 1.00 | 11.65 |
| ATOM | 639 | CB | ASN | A | 614 | 15.955 | 9.702 | 17.516 | 1.00 | 19.08 |
| ATOM | 640 | CG | ASN | A | 614 | 15.631 | 9.295 | 16.090 | 1.00 | 17.63 |
| ATOM | 641 | OD1 | ASN | A | 614 | 16.529 | 9.151 | 15.253 | 1.00 | 16.40 |
| ATOM | 642 | ND2 | ASN | A | 614 | 14.349 | 9.115 | 15.802 | 1.00 | 11.39 |
| ATOM | 643 | C | ASN | A | 614 | 13.933 | 8.690 | 18.644 | 1.00 | 11.53 |
| ATOM | 644 | O | ASN | A | 614 | 13.298 | 7.647 | 18.493 | 1.00 | 9.32 |
| ATOM | 645 | N | TRP | A | 615 | 13.342 | 9.850 | 18.890 | 1.00 | 8.95 |
| ATOM | 646 | CA | TRP | A | 615 | 11.893 | 9.929 | 19.015 | 1.00 | 9.02 |
| ATOM | 647 | CB | TRP | A | 615 | 11.457 | 11.397 | 19.127 | 1.00 | 9.00 |
| ATOM | 648 | CG | TRP | A | 615 | 10.002 | 11.638 | 18.809 | 1.00 | 8.75 |
| ATOM | 649 | CD2 | TRP | A | 615 | 8.860 | 11.228 | 19.581 | 1.00 | 10.58 |
| ATOM | 650 | CE2 | TRP | A | 615 | 7.709 | 11.655 | 18.883 | 1.00 | 12.68 |
| ATOM | 651 | CE3 | TRP | A | 615 | 8.698 | 10.542 | 20.789 | 1.00 | 12.70 |
| ATOM | 652 | CD1 | TRP | A | 615 | 9.504 | 12.279 | 17.709 | 1.00 | 11.21 |
| ATOM | 653 | NE1 | TRP | A | 615 | 8.132 | 12.292 | 17.747 | 1.00 | 11.25 |
| ATOM | 654 | CZ2 | TRP | A | 615 | 6.414 | 11.418 | 19.354 | 1.00 | 12.28 |
| ATOM | 655 | CZ3 | TRP | A | 615 | 7.410 | 10.307 | 21.255 | 1.00 | 18.15 |
| ATOM | 656 | CH2 | TRP | A | 615 | 6.286 | 10.744 | 20.536 | 1.00 | 13.05 |
| ATOM | 657 | C | TRP | A | 615 | 11.090 | 9.237 | 17.910 | 1.00 | 8.08 |
| ATOM | 658 | O | TRP | A | 615 | 10.093 | 8.579 | 18.197 | 1.00 | 9.24 |
| ATOM | 659 | N | ARG | A | 616 | 11.514 | 9.365 | 16.656 | 1.00 | 9.61 |
| ATOM | 660 | CA | ARG | A | 616 | 10.750 | 8.759 | 15.570 | 1.00 | 11.24 |
| ATOM | 661 | CB | ARG | A | 616 | 11.346 | 9.129 | 14.206 | 1.00 | 9.33 |
| ATOM | 662 | CG | ARG | A | 616 | 11.241 | 10.630 | 13.885 | 1.00 | 13.33 |
| ATOM | 663 | CD | ARG | A | 616 | 9.817 | 11.169 | 14.120 | 1.00 | 11.22 |
| ATOM | 664 | NE | ARG | A | 616 | 9.705 | 12.598 | 13.823 | 1.00 | 13.61 |
| ATOM | 665 | CZ | ARG | A | 616 | 9.119 | 13.103 | 12.741 | 1.00 | 15.86 |
| ATOM | 666 | NH1 | ARG | A | 616 | 8.577 | 12.300 | 11.834 | 1.00 | 15.89 |
| ATOM | 667 | NH2 | ARG | A | 616 | 9.073 | 14.419 | 12.564 | 1.00 | 19.53 |
| ATOM | 668 | C | ARG | A | 616 | 10.607 | 7.250 | 15.705 | 1.00 | 9.88 |
| ATOM | 669 | O | ARG | A | 616 | 9.575 | 6.692 | 15.328 | 1.00 | 10.40 |
| ATOM | 670 | N | HIS | A | 617 | 11.625 | 6.582 | 16.242 | 1.00 | 7.87 |
| ATOM | 671 | CA | HIS | A | 617 | 11.514 | 5.141 | 16.419 | 1.00 | 7.37 |
| ATOM | 672 | CB | HIS | A | 617 | 12.862 | 4.523 | 16.782 | 1.00 | 11.98 |
| ATOM | 673 | CG | HIS | A | 617 | 12.742 | 3.163 | 17.390 | 1.00 | 8.70 |
| ATOM | 674 | CD2 | HIS | A | 617 | 13.043 | 2.715 | 18.632 | 1.00 | 9.51 |
| ATOM | 675 | ND1 | HIS | A | 617 | 12.206 | 2.088 | 16.713 | 1.00 | 8.99 |
| ATOM | 676 | CE1 | HIS | A | 617 | 12.183 | 1.036 | 17.510 | 1.00 | 11.83 |
| ATOM | 677 | NE2 | HIS | A | 617 | 12.684 | 1.391 | 18.681 | 1.00 | 10.58 |
| ATOM | 678 | C | HIS | A | 617 | 10.488 | 4.816 | 17.506 | 1.00 | 8.63 |
| ATOM | 679 | O | HIS | A | 617 | 9.692 | 3.888 | 17.361 | 1.00 | 10.24 |
| ATOM | 680 | N | ALA | A | 618 | 10.498 | 5.586 | 18.591 | 1.00 | 7.45 |
| ATOM | 681 | CA | ALA | A | 618 | 9.549 | 5.358 | 19.687 | 1.00 | 8.54 |
| ATOM | 682 | CB | ALA | A | 618 | 9.887 | 6.261 | 20.864 | 1.00 | 11.31 |
| ATOM | 683 | C | ALA | A | 618 | 8.126 | 5.637 | 19.210 | 1.00 | 8.39 |
| ATOM | 684 | O | ALA | A | 618 | 7.184 | 4.900 | 19.523 | 1.00 | 9.03 |
| ATOM | 685 | N | PHE | A | 619 | 7.981 | 6.725 | 18.465 | 1.00 | 8.88 |
| ATOM | 686 | CA | PHE | A | 619 | 6.695 | 7.118 | 17.911 | 1.00 | 9.63 |
| ATOM | 687 | CB | PHE | A | 619 | 6.850 | 8.418 | 17.119 | 1.00 | 12.92 |
| ATOM | 688 | CG | PHE | A | 619 | 5.618 | 8.809 | 16.357 | 1.00 | 10.44 |
| ATOM | 689 | CD1 | PHE | A | 619 | 4.432 | 9.066 | 17.021 | 1.00 | 18.21 |
| ATOM | 690 | CD2 | PHE | A | 619 | 5.646 | 8.914 | 14.974 | 1.00 | 12.41 |
| ATOM | 691 | CE1 | PHE | A | 619 | 3.291 | 9.412 | 16.324 | 1.00 | 14.74 |
| ATOM | 692 | CE2 | PHE | A | 619 | 4.508 | 9.260 | 14.270 | 1.00 | 13.16 |
| ATOM | 693 | CZ | PHE | A | 619 | 3.328 | 9.512 | 14.947 | 1.00 | 11.01 |
| ATOM | 694 | C | PHE | A | 619 | 6.150 | 6.017 | 16.996 | 1.00 | 9.52 |
| ATOM | 695 | O | PHE | A | 619 | 4.975 | 5.654 | 17.079 | 1.00 | 9.30 |
| ATOM | 696 | N | ASN | A | 620 | 7.001 | 5.490 | 16.119 | 1.00 | 10.15 |
| ATOM | 697 | CA | ASN | A | 620 | 6.583 | 4.428 | 15.204 | 1.00 | 11.34 |
| ATOM | 698 | CB | ASN | A | 620 | 7.694 | 4.123 | 14.194 | 1.00 | 9.10 |
| ATOM | 699 | CG | ASN | A | 620 | 7.658 | 5.052 | 12.999 | 1.00 | 17.16 |
| ATOM | 700 | OD1 | ASN | A | 620 | 8.665 | 5.656 | 12.634 | 1.00 | 19.51 |
| ATOM | 701 | ND2 | ASN | A | 620 | 6.490 | 5.166 | 12.382 | 1.00 | 12.88 |
| ATOM | 702 | C | ASN | A | 620 | 6.206 | 3.161 | 15.956 | 1.00 | 9.23 |
| ATOM | 703 | O | ASN | A | 620 | 5.273 | 2.457 | 15.574 | 1.00 | 10.76 |
| ATOM | 704 | N | THR | A | 621 | 6.936 | 2.871 | 17.026 | 1.00 | 8.56 |
| ATOM | 705 | CA | THR | A | 621 | 6.650 | 1.688 | 17.819 | 1.00 | 9.52 |
| ATOM | 706 | CB | THR | A | 621 | 7.707 | 1.509 | 18.928 | 1.00 | 8.96 |
| ATOM | 707 | OG1 | THR | A | 621 | 9.010 | 1.404 | 18.328 | 1.00 | 9.14 |
| ATOM | 708 | CG2 | THR | A | 621 | 7.428 | 0.244 | 19.736 | 1.00 | 7.93 |
| ATOM | 709 | C | THR | A | 621 | 5.252 | 1.821 | 18.425 | 1.00 | 10.48 |
| ATOM | 710 | O | THR | A | 621 | 4.455 | 0.879 | 18.382 | 1.00 | 10.11 |
| ATOM | 711 | N | ALA | A | 622 | 4.954 | 2.995 | 18.978 | 1.00 | 8.32 |
| ATOM | 712 | CA | ALA | A | 622 | 3.644 | 3.244 | 19.573 | 1.00 | 9.28 |
| ATOM | 713 | CB | ALA | A | 622 | 3.642 | 4.583 | 20.294 | 1.00 | 9.90 |
| ATOM | 714 | C | ALA | A | 622 | 2.567 | 3.224 | 18.488 | 1.00 | 10.02 |
| ATOM | 715 | O | ALA | A | 622 | 1.447 | 2.769 | 18.721 | 1.00 | 8.06 |
| ATOM | 716 | N | GLN | A | 623 | 2.902 | 3.708 | 17.297 | 1.00 | 7.82 |
| ATOM | 717 | CA | GLN | A | 623 | 1.927 | 3.708 | 16.211 | 1.00 | 10.17 |
| ATOM | 718 | CB | GLN | A | 623 | 2.456 | 4.487 | 15.003 | 1.00 | 8.39 |
| ATOM | 719 | CG | GLN | A | 623 | 1.473 | 4.578 | 13.825 | 1.00 | 8.23 |
| ATOM | 720 | CD | GLN | A | 623 | 1.444 | 3.319 | 12.967 | 1.00 | 13.63 |
| ATOM | 721 | OE1 | GLN | A | 623 | 2.484 | 2.730 | 12.682 | 1.00 | 12.75 |
| ATOM | 722 | NE2 | GLN | A | 623 | 0.258 | 2.919 | 12.535 | 1.00 | 11.69 |
| ATOM | 723 | C | GLN | A | 623 | 1.597 | 2.278 | 15.806 | 1.00 | 10.35 |
| ATOM | 724 | O | GLN | A | 623 | 0.449 | 1.962 | 15.501 | 1.00 | 9.61 |
| ATOM | 725 | N | CYS | A | 624 | 2.601 | 1.406 | 15.802 | 1.00 | 9.08 |
| ATOM | 726 | CA | CYS | A | 624 | 2.355 | 0.017 | 15.439 | 1.00 | 9.34 |
| ATOM | 727 | CB | CYS | A | 624 | 3.679 | −0.740 | 15.273 | 1.00 | 11.00 |
| ATOM | 728 | SG | CYS | A | 624 | 3.481 | −2.446 | 14.713 | 1.00 | 11.71 |
| ATOM | 729 | C | CYS | A | 624 | 1.495 | −0.625 | 16.529 | 1.00 | 10.17 |
| ATOM | 730 | O | CYS | A | 624 | 0.631 | −1.448 | 16.239 | 1.00 | 10.64 |
| ATOM | 731 | N | MET | A | 625 | 1.728 | −0.238 | 17.781 | 1.00 | 9.06 |
| ATOM | 732 | CA | MET | A | 625 | 0.933 | −0.764 | 18.892 | 1.00 | 9.38 |
| ATOM | 733 | CB | MET | A | 625 | 1.418 | −0.180 | 20.225 | 1.00 | 8.21 |
| ATOM | 734 | CG | MET | A | 625 | 0.692 | −0.718 | 21.466 | 1.00 | 8.16 |
| ATOM | 735 | SD | MET | A | 625 | 0.904 | −2.512 | 21.728 | 1.00 | 11.88 |
| ATOM | 736 | CE | MET | A | 625 | 2.600 | −2.586 | 22.309 | 1.00 | 11.24 |
| ATOM | 737 | C | MET | A | 625 | −0.529 | −0.376 | 18.661 | 1.00 | 9.31 |
| ATOM | 738 | O | MET | A | 625 | −1.434 | −1.208 | 18.757 | 1.00 | 10.06 |
| ATOM | 739 | N | PHE | A | 626 | −0.757 | 0.896 | 18.353 | 1.00 | 9.47 |
| ATOM | 740 | CA | PHE | A | 626 | −2.112 | 1.377 | 18.092 | 1.00 | 9.60 |
| ATOM | 741 | CB | PHE | A | 626 | −2.077 | 2.859 | 17.725 | 1.00 | 9.70 |
| ATOM | 742 | CG | PHE | A | 626 | −3.432 | 3.451 | 17.461 | 1.00 | 7.25 |
| ATOM | 743 | CD1 | PHE | A | 626 | −4.241 | 3.858 | 18.509 | 1.00 | 13.86 |
| ATOM | 744 | CD2 | PHE | A | 626 | −3.897 | 3.598 | 16.163 | 1.00 | 13.47 |
| ATOM | 745 | CE1 | PHE | A | 626 | −5.491 | 4.411 | 18.267 | 1.00 | 18.88 |
| ATOM | 746 | CE2 | PHE | A | 626 | −5.144 | 4.147 | 15.917 | 1.00 | 11.81 |
| ATOM | 747 | CZ | PHE | A | 626 | −5.942 | 4.552 | 16.970 | 1.00 | 16.53 |
| ATOM | 748 | C | PHE | A | 626 | −2.737 | 0.579 | 16.940 | 1.00 | 10.98 |
| ATOM | 749 | O | PHE | A | 626 | −3.862 | 0.084 | 17.047 | 1.00 | 12.71 |
| ATOM | 750 | N | ALA | A | 627 | −1.999 | 0.455 | 15.838 | 1.00 | 7.24 |
| ATOM | 751 | CA | ALA | A | 627 | −2.486 | −0.279 | 14.675 | 1.00 | 9.31 |
| ATOM | 752 | CB | ALA | A | 627 | −1.454 | −0.220 | 13.549 | 1.00 | 14.42 |
| ATOM | 753 | C | ALA | A | 627 | −2.803 | −1.734 | 15.024 | 1.00 | 11.79 |
| ATOM | 754 | O | ALA | A | 627 | −3.815 | −2.276 | 14.583 | 1.00 | 11.59 |
| ATOM | 755 | N | ALA | A | 628 | −1.945 | −2.361 | 15.824 | 1.00 | 10.63 |
| ATOM | 756 | CA | ALA | A | 628 | −2.158 | −3.749 | 16.219 | 1.00 | 11.00 |
| ATOM | 757 | CB | ALA | A | 628 | −0.934 | −4.280 | 16.961 | 1.00 | 11.43 |
| ATOM | 758 | C | ALA | A | 628 | −3.398 | −3.877 | 17.099 | 1.00 | 11.16 |
| ATOM | 759 | O | ALA | A | 628 | −4.152 | −4.845 | 16.994 | 1.00 | 12.30 |
| ATOM | 760 | N | LEU | A | 629 | −3.608 | −2.896 | 17.970 | 1.00 | 9.63 |
| ATOM | 761 | CA | LEU | A | 629 | −4.765 | −2.924 | 18.856 | 1.00 | 9.96 |
| ATOM | 762 | CB | LEU | A | 629 | −4.627 | −1.858 | 19.945 | 1.00 | 12.79 |
| ATOM | 763 | CG | LEU | A | 629 | −3.538 | −2.115 | 20.990 | 1.00 | 10.87 |
| ATOM | 764 | CD1 | LEU | A | 629 | −3.286 | −0.859 | 21.792 | 1.00 | 11.38 |
| ATOM | 765 | CD2 | LEU | A | 629 | −3.956 | −3.264 | 21.897 | 1.00 | 14.99 |
| ATOM | 766 | C | LEU | A | 629 | −6.051 | −2.692 | 18.073 | 1.00 | 12.31 |
| ATOM | 767 | O | LEU | A | 629 | −7.084 | −3.300 | 18.364 | 1.00 | 11.63 |
| ATOM | 768 | N | LYS | A | 630 | −5.973 | −1.822 | 17.070 | 1.00 | 11.64 |
| ATOM | 769 | CA | LYS | A | 630 | −7.125 | −1.479 | 16.242 | 1.00 | 12.87 |
| ATOM | 770 | CB | LYS | A | 630 | −6.984 | −0.037 | 15.738 | 1.00 | 15.53 |
| ATOM | 771 | CG | LYS | A | 630 | −7.084 | 1.030 | 16.820 | 1.00 | 13.30 |
| ATOM | 772 | CD | LYS | A | 630 | −8.495 | 1.104 | 17.385 | 1.00 | 19.60 |
| ATOM | 773 | CE | LYS | A | 630 | −8.698 | 2.349 | 18.245 | 1.00 | 18.93 |
| ATOM | 774 | NZ | LYS | A | 630 | −7.877 | 2.334 | 19.481 | 1.00 | 40.94 |
| ATOM | 775 | C | LYS | A | 630 | −7.323 | −2.422 | 15.049 | 1.00 | 13.54 |
| ATOM | 776 | O | LYS | A | 630 | −8.122 | −3.350 | 15.107 | 1.00 | 12.81 |
| ATOM | 777 | N | ALA | A | 631 | −6.597 | −2.170 | 13.967 | 1.00 | 13.05 |
| ATOM | 778 | CA | ALA | A | 631 | −6.701 | −2.997 | 12.770 | 1.00 | 14.94 |
| ATOM | 779 | CB | ALA | A | 631 | −5.763 | −2.460 | 11.688 | 1.00 | 17.58 |
| ATOM | 780 | C | ALA | A | 631 | −6.374 | −4.461 | 13.068 | 1.00 | 16.22 |
| ATOM | 781 | O | ALA | A | 631 | −6.960 | −5.375 | 12.479 | 1.00 | 15.44 |
| ATOM | 782 | N | GLY | A | 632 | −5.439 | −4.670 | 13.990 | 1.00 | 12.88 |
| ATOM | 783 | CA | GLY | A | 632 | −5.028 | −6.014 | 14.359 | 1.00 | 13.10 |
| ATOM | 784 | C | GLY | A | 632 | −5.995 | −6.711 | 15.298 | 1.00 | 13.12 |
| ATOM | 785 | O | GLY | A | 632 | −5.850 | −7.902 | 15.576 | 1.00 | 15.03 |
| ATOM | 786 | N | LYS | A | 633 | −6.968 | −5.959 | 15.803 | 1.00 | 13.75 |
| ATOM | 787 | CA | LYS | A | 633 | −7.997 | −6.497 | 16.692 | 1.00 | 14.20 |
| ATOM | 788 | CB | LYS | A | 633 | −8.815 | −7.566 | 15.954 | 1.00 | 13.72 |
| ATOM | 789 | CG | LYS | A | 633 | −9.438 | −7.109 | 14.631 | 1.00 | 23.64 |
| ATOM | 790 | CD | LYS | A | 633 | −10.532 | −6.072 | 14.834 | 1.00 | 30.20 |
| ATOM | 791 | CE | LYS | A | 633 | −11.182 | −5.672 | 13.510 | 1.00 | 34.87 |
| ATOM | 792 | NZ | LYS | A | 633 | −10.227 | −4.989 | 12.594 | 1.00 | 46.21 |
| ATOM | 793 | C | LYS | A | 633 | −7.464 | −7.085 | 17.997 | 1.00 | 14.18 |
| ATOM | 794 | O | LYS | A | 633 | −8.048 | −8.024 | 18.540 | 1.00 | 14.72 |
| ATOM | 795 | N | ILE | A | 634 | −6.366 | −6.536 | 18.507 | 1.00 | 13.07 |
| ATOM | 796 | CA | ILE | A | 634 | −5.801 | −7.037 | 19.755 | 1.00 | 12.44 |
| ATOM | 797 | CB | ILE | A | 634 | −4.262 | −6.819 | 19.799 | 1.00 | 13.52 |
| ATOM | 798 | CG2 | ILE | A | 634 | −3.709 | −7.236 | 21.156 | 1.00 | 14.73 |
| ATOM | 799 | CG1 | ILE | A | 634 | −3.581 | −7.632 | 18.694 | 1.00 | 14.10 |
| ATOM | 800 | CD1 | ILE | A | 634 | −3.757 | −9.128 | 18.829 | 1.00 | 14.44 |
| ATOM | 801 | C | ILE | A | 634 | −6.439 | −6.346 | 20.967 | 1.00 | 12.23 |
| ATOM | 802 | O | ILE | A | 634 | −6.456 | −6.894 | 22.067 | 1.00 | 12.06 |
| ATOM | 803 | N | GLN | A | 635 | −6.990 | −5.155 | 20.752 | 1.00 | 13.17 |
| ATOM | 804 | CA | GLN | A | 635 | −7.597 | −4.377 | 21.834 | 1.00 | 12.59 |
| ATOM | 805 | CB | GLN | A | 635 | −8.330 | −3.158 | 21.267 | 1.00 | 14.77 |
| ATOM | 806 | CG | GLN | A | 635 | −8.796 | −2.180 | 22.345 | 1.00 | 17.07 |
| ATOM | 807 | CD | GLN | A | 635 | −9.479 | −0.949 | 21.778 | 1.00 | 17.87 |
| ATOM | 808 | OE1 | GLN | A | 635 | −9.080 | −0.425 | 20.741 | 1.00 | 15.93 |
| ATOM | 809 | NE2 | GLN | A | 635 | −10.509 | −0.473 | 22.471 | 1.00 | 27.59 |
| ATOM | 810 | C | GLN | A | 635 | −8.549 | −5.127 | 22.762 | 1.00 | 13.46 |
| ATOM | 811 | O | GLN | A | 635 | −8.382 | −5.107 | 23.984 | 1.00 | 12.66 |
| ATOM | 812 | N | ASN | A | 636 | −9.554 | −5.778 | 22.188 | 1.00 | 12.36 |
| ATOM | 813 | CA | ASN | A | 636 | −10.533 | −6.493 | 22.996 | 1.00 | 15.16 |
| ATOM | 814 | CB | ASN | A | 636 | −11.736 | −6.872 | 22.130 | 1.00 | 13.54 |
| ATOM | 815 | CG | ASN | A | 636 | −12.508 | −5.655 | 21.658 | 1.00 | 19.96 |
| ATOM | 816 | OD1 | ASN | A | 636 | −12.211 | −4.526 | 22.059 | 1.00 | 19.43 |
| ATOM | 817 | ND2 | ASN | A | 636 | −13.506 | −5.874 | 20.810 | 1.00 | 20.88 |
| ATOM | 818 | C | ASN | A | 636 | −10.002 | −7.708 | 23.743 | 1.00 | 14.39 |
| ATOM | 819 | O | ASN | A | 636 | −10.720 | −8.312 | 24.539 | 1.00 | 17.61 |
| ATOM | 820 | N | LYS | A | 637 | −8.744 | −8.060 | 23.500 | 1.00 | 13.69 |
| ATOM | 821 | CA | LYS | A | 637 | −8.132 | −9.194 | 24.180 | 1.00 | 13.94 |
| ATOM | 822 | CB | LYS | A | 637 | −7.124 | −9.893 | 23.262 | 1.00 | 13.19 |
| ATOM | 823 | CG | LYS | A | 637 | −7.667 | −10.328 | 21.910 | 1.00 | 16.41 |
| ATOM | 824 | CD | LYS | A | 637 | −6.599 | −11.079 | 21.128 | 1.00 | 18.79 |
| ATOM | 825 | CE | LYS | A | 637 | −7.110 | −11.542 | 19.773 | 1.00 | 21.00 |
| ATOM | 826 | NZ | LYS | A | 637 | −8.266 | −12.467 | 19.893 | 1.00 | 27.72 |
| ATOM | 827 | C | LYS | A | 637 | −7.400 | −8.708 | 25.435 | 1.00 | 14.29 |
| ATOM | 828 | O | LYS | A | 637 | −6.930 | −9.514 | 26.237 | 1.00 | 15.91 |
| ATOM | 829 | N | LEU | A | 638 | −7.316 | −7.391 | 25.611 | 1.00 | 13.88 |
| ATOM | 830 | CA | LEU | A | 638 | −6.602 | −6.828 | 26.757 | 1.00 | 14.02 |
| ATOM | 831 | CB | LEU | A | 638 | −5.426 | −5.988 | 26.256 | 1.00 | 14.42 |
| ATOM | 832 | CG | LEU | A | 638 | −4.496 | −6.595 | 25.201 | 1.00 | 13.65 |
| ATOM | 833 | CD1 | LEU | A | 638 | −3.405 | −5.589 | 24.860 | 1.00 | 13.98 |
| ATOM | 834 | CD2 | LEU | A | 638 | −3.881 | −7.885 | 25.718 | 1.00 | 12.70 |
| ATOM | 835 | C | LEU | A | 638 | −7.464 | −5.973 | 27.687 | 1.00 | 13.83 |
| ATOM | 836 | O | LEU | A | 638 | −8.551 | −5.529 | 27.313 | 1.00 | 16.24 |
| ATOM | 837 | N | THR | A | 639 | −6.968 | −5.745 | 28.902 | 1.00 | 13.04 |
| ATOM | 838 | CA | THR | A | 639 | −7.665 | −4.913 | 29.885 | 1.00 | 12.97 |
| ATOM | 839 | CB | THR | A | 639 | −7.225 | −5.244 | 31.323 | 1.00 | 9.72 |
| ATOM | 840 | OG1 | THR | A | 639 | −5.883 | −4.777 | 31.534 | 1.00 | 11.58 |
| ATOM | 841 | CG2 | THR | A | 639 | −7.284 | −6.752 | 31.574 | 1.00 | 13.01 |
| ATOM | 842 | C | THR | A | 639 | −7.320 | −3.443 | 29.627 | 1.00 | 11.14 |
| ATOM | 843 | O | THR | A | 639 | −6.369 | −3.147 | 28.910 | 1.00 | 12.39 |
| ATOM | 844 | N | ASP | A | 640 | −8.082 | −2.524 | 30.216 | 1.00 | 11.83 |
| ATOM | 845 | CA | ASP | A | 640 | −7.814 | −1.098 | 30.027 | 1.00 | 11.03 |
| ATOM | 846 | CB | ASP | A | 640 | −8.846 | −0.243 | 30.774 | 1.00 | 9.58 |
| ATOM | 847 | CG | ASP | A | 640 | −10.238 | −0.368 | 30.191 | 1.00 | 23.27 |
| ATOM | 848 | OD1 | ASP | A | 640 | −10.357 | −0.828 | 29.037 | 1.00 | 21.74 |
| ATOM | 849 | OD2 | ASP | A | 640 | −11.211 | 0.011 | 30.879 | 1.00 | 18.96 |
| ATOM | 850 | C | ASP | A | 640 | −6.419 | −0.724 | 30.519 | 1.00 | 10.85 |
| ATOM | 851 | O | ASP | A | 640 | −5.708 | 0.058 | 29.881 | 1.00 | 11.05 |
| ATOM | 852 | N | LEU | A | 641 | −6.031 | −1.290 | 31.655 | 1.00 | 11.16 |
| ATOM | 853 | CA | LEU | A | 641 | −4.727 | −1.004 | 32.235 | 1.00 | 10.51 |
| ATOM | 854 | CB | LEU | A | 641 | −4.628 | −1.606 | 33.640 | 1.00 | 13.05 |
| ATOM | 855 | CG | LEU | A | 641 | −5.633 | −1.081 | 34.674 | 1.00 | 13.28 |
| ATOM | 856 | CD1 | LEU | A | 641 | −5.383 | −1.762 | 36.005 | 1.00 | 14.91 |
| ATOM | 857 | CD2 | LEU | A | 641 | −5.506 | 0.432 | 34.813 | 1.00 | 12.98 |
| ATOM | 858 | C | LEU | A | 641 | −3.593 | −1.527 | 31.359 | 1.00 | 10.02 |
| ATOM | 859 | O | LEU | A | 641 | −2.550 | −0.883 | 31.237 | 1.00 | 9.79 |
| ATOM | 860 | N | GLU | A | 642 | −3.791 | −2.692 | 30.748 | 1.00 | 9.49 |
| ATOM | 861 | CA | GLU | A | 642 | −2.756 | −3.253 | 29.886 | 1.00 | 9.55 |
| ATOM | 862 | CB | GLU | A | 642 | −3.120 | −4.687 | 29.493 | 1.00 | 13.24 |
| ATOM | 863 | CG | GLU | A | 642 | −3.215 | −5.605 | 30.705 | 1.00 | 17.56 |
| ATOM | 864 | CD | GLU | A | 642 | −3.645 | −7.021 | 30.373 | 1.00 | 14.47 |
| ATOM | 865 | OE1 | GLU | A | 642 | −4.411 | −7.212 | 29.404 | 1.00 | 12.63 |
| ATOM | 866 | OE2 | GLU | A | 642 | −3.233 | −7.949 | 31.108 | 1.00 | 15.36 |
| ATOM | 867 | C | GLU | A | 642 | −2.568 | −2.366 | 28.656 | 1.00 | 9.56 |
| ATOM | 868 | O | GLU | A | 642 | −1.442 | −2.135 | 28.217 | 1.00 | 10.36 |
| ATOM | 869 | N | ILE | A | 643 | −3.665 | −1.847 | 28.112 | 1.00 | 9.98 |
| ATOM | 870 | CA | ILE | A | 643 | −3.585 | −0.978 | 26.945 | 1.00 | 11.53 |
| ATOM | 871 | CB | ILE | A | 643 | −4.992 | −0.683 | 26.391 | 1.00 | 12.52 |
| ATOM | 872 | CG2 | ILE | A | 643 | −4.915 | 0.367 | 25.281 | 1.00 | 11.83 |
| ATOM | 873 | CG1 | ILE | A | 643 | −5.614 | −1.984 | 25.885 | 1.00 | 11.82 |
| ATOM | 874 | CD1 | ILE | A | 643 | −7.089 | −1.872 | 25.553 | 1.00 | 18.49 |
| ATOM | 875 | C | ILE | A | 643 | −2.883 | 0.323 | 27.339 | 1.00 | 9.56 |
| ATOM | 876 | O | ILE | A | 643 | −1.993 | 0.806 | 26.635 | 1.00 | 10.76 |
| ATOM | 877 | N | LEU | A | 644 | −3.278 | 0.880 | 28.479 | 1.00 | 9.68 |
| ATOM | 878 | CA | LEU | A | 644 | −2.663 | 2.105 | 28.974 | 1.00 | 9.78 |
| ATOM | 879 | CB | LEU | A | 644 | −3.285 | 2.482 | 30.321 | 1.00 | 11.15 |
| ATOM | 880 | CG | LEU | A | 644 | −2.658 | 3.643 | 31.093 | 1.00 | 14.02 |
| ATOM | 881 | CD1 | LEU | A | 644 | −2.853 | 4.946 | 30.328 | 1.00 | 14.15 |
| ATOM | 882 | CD2 | LEU | A | 644 | −3.315 | 3.735 | 32.471 | 1.00 | 14.10 |
| ATOM | 883 | C | LEU | A | 644 | −1.152 | 1.899 | 29.139 | 1.00 | 9.98 |
| ATOM | 884 | O | LEU | A | 644 | −0.342 | 2.720 | 28.694 | 1.00 | 9.97 |
| ATOM | 885 | N | ALA | A | 645 | −0.778 | 0.788 | 29.766 | 1.00 | 8.98 |
| ATOM | 886 | CA | ALA | A | 645 | 0.634 | 0.492 | 30.008 | 1.00 | 8.84 |
| ATOM | 887 | CB | ALA | A | 645 | 0.763 | −0.724 | 30.928 | 1.00 | 8.17 |
| ATOM | 888 | C | ALA | A | 645 | 1.414 | 0.256 | 28.718 | 1.00 | 9.39 |
| ATOM | 889 | O | ALA | A | 645 | 2.556 | 0.687 | 28.598 | 1.00 | 9.92 |
| ATOM | 890 | N | LEU | A | 646 | 0.799 | −0.430 | 27.761 | 1.00 | 8.76 |
| ATOM | 891 | CA | LEU | A | 646 | 1.462 | −0.708 | 26.488 | 1.00 | 8.34 |
| ATOM | 892 | CB | LEU | A | 646 | 0.607 | −1.652 | 25.644 | 1.00 | 10.30 |
| ATOM | 893 | CG | LEU | A | 646 | 0.541 | −3.101 | 26.128 | 1.00 | 11.47 |
| ATOM | 894 | CD1 | LEU | A | 646 | −0.557 | −3.832 | 25.379 | 1.00 | 15.28 |
| ATOM | 895 | CD2 | LEU | A | 646 | 1.879 | −3.779 | 25.906 | 1.00 | 10.48 |
| ATOM | 896 | C | LEU | A | 646 | 1.761 | 0.550 | 25.682 | 1.00 | 7.55 |
| ATOM | 897 | O | LEU | A | 646 | 2.837 | 0.676 | 25.094 | 1.00 | 9.05 |
| ATOM | 898 | N | LEU | A | 647 | 0.812 | 1.479 | 25.643 | 1.00 | 7.57 |
| ATOM | 899 | CA | LEU | A | 647 | 1.020 | 2.713 | 24.889 | 1.00 | 8.87 |
| ATOM | 900 | CB | LEU | A | 647 | −0.283 | 3.516 | 24.803 | 1.00 | 7.82 |
| ATOM | 901 | CG | LEU | A | 647 | −0.214 | 4.722 | 23.862 | 1.00 | 10.06 |
| ATOM | 902 | CD1 | LEU | A | 647 | 0.194 | 4.251 | 22.470 | 1.00 | 13.81 |
| ATOM | 903 | CD2 | LEU | A | 647 | −1.560 | 5.437 | 23.820 | 1.00 | 12.05 |
| ATOM | 904 | C | LEU | A | 647 | 2.120 | 3.550 | 25.540 | 1.00 | 8.12 |
| ATOM | 905 | O | LEU | A | 647 | 3.011 | 4.070 | 24.863 | 1.00 | 8.78 |
| ATOM | 906 | N | ILE | A | 648 | 2.056 | 3.676 | 26.860 | 1.00 | 9.66 |
| ATOM | 907 | CA | ILE | A | 648 | 3.065 | 4.429 | 27.597 | 1.00 | 8.86 |
| ATOM | 908 | CB | ILE | A | 648 | 2.692 | 4.501 | 29.100 | 1.00 | 10.81 |
| ATOM | 909 | CG2 | ILE | A | 648 | 3.851 | 5.063 | 29.917 | 1.00 | 7.70 |
| ATOM | 910 | CG1 | ILE | A | 648 | 1.439 | 5.364 | 29.266 | 1.00 | 11.45 |
| ATOM | 911 | CD1 | ILE | A | 648 | 0.921 | 5.437 | 30.688 | 1.00 | 9.57 |
| ATOM | 912 | C | ILE | A | 648 | 4.427 | 3.756 | 27.424 | 1.00 | 8.38 |
| ATOM | 913 | O | ILE | A | 648 | 5.439 | 4.427 | 27.180 | 1.00 | 9.63 |
| ATOM | 914 | N | ALA | A | 649 | 4.456 | 2.430 | 27.536 | 1.00 | 9.32 |
| ATOM | 915 | CA | ALA | A | 649 | 5.710 | 1.697 | 27.388 | 1.00 | 9.98 |
| ATOM | 916 | CB | ALA | A | 649 | 5.506 | 0.214 | 27.703 | 1.00 | 11.64 |
| ATOM | 917 | C | ALA | A | 649 | 6.285 | 1.853 | 25.989 | 1.00 | 9.64 |
| ATOM | 918 | O | ALA | A | 649 | 7.471 | 2.126 | 25.831 | 1.00 | 10.34 |
| ATOM | 919 | N | ALA | A | 650 | 5.449 | 1.680 | 24.971 | 1.00 | 11.02 |
| ATOM | 920 | CA | ALA | A | 650 | 5.914 | 1.814 | 23.591 | 1.00 | 9.43 |
| ATOM | 921 | CB | ALA | A | 650 | 4.747 | 1.618 | 22.617 | 1.00 | 8.79 |
| ATOM | 922 | C | ALA | A | 650 | 6.561 | 3.183 | 23.359 | 1.00 | 8.11 |
| ATOM | 923 | O | ALA | A | 650 | 7.628 | 3.292 | 22.748 | 1.00 | 9.60 |
| ATOM | 924 | N | LEU | A | 651 | 5.914 | 4.233 | 23.851 | 1.00 | 9.58 |
| ATOM | 925 | CA | LEU | A | 651 | 6.443 | 5.582 | 23.679 | 1.00 | 10.27 |
| ATOM | 926 | CB | LEU | A | 651 | 5.383 | 6.613 | 24.066 | 1.00 | 9.84 |
| ATOM | 927 | CG | LEU | A | 651 | 4.198 | 6.717 | 23.101 | 1.00 | 6.24 |
| ATOM | 928 | CD1 | LEU | A | 651 | 3.038 | 7.432 | 23.772 | 1.00 | 14.57 |
| ATOM | 929 | CD2 | LEU | A | 651 | 4.637 | 7.463 | 21.846 | 1.00 | 11.43 |
| ATOM | 930 | C | LEU | A | 651 | 7.706 | 5.848 | 24.488 | 1.00 | 10.46 |
| ATOM | 931 | O | LEU | A | 651 | 8.573 | 6.611 | 24.066 | 1.00 | 9.75 |
| ATOM | 932 | N | SER | A | 652 | 7.807 | 5.194 | 25.640 | 1.00 | 9.58 |
| ATOM | 933 | CA | SER | A | 652 | 8.935 | 5.394 | 26.542 | 1.00 | 10.30 |
| ATOM | 934 | CB | SER | A | 652 | 8.422 | 5.453 | 27.983 | 1.00 | 9.30 |
| ATOM | 935 | OG | SER | A | 652 | 7.367 | 6.379 | 28.133 | 1.00 | 11.78 |
| ATOM | 936 | C | SER | A | 652 | 10.026 | 4.334 | 26.504 | 1.00 | 8.64 |
| ATOM | 937 | O | SER | A | 652 | 11.068 | 4.520 | 27.125 | 1.00 | 9.90 |
| ATOM | 938 | N | HIS | A | 653 | 9.805 | 3.249 | 25.765 | 1.00 | 9.64 |
| ATOM | 939 | CA | HIS | A | 653 | 10.739 | 2.128 | 25.769 | 1.00 | 9.99 |
| ATOM | 940 | CB | HIS | A | 653 | 10.164 | 0.963 | 24.952 | 1.00 | 8.26 |
| ATOM | 941 | CG | HIS | A | 653 | 10.546 | 0.980 | 23.510 | 1.00 | 8.55 |
| ATOM | 942 | CD2 | HIS | A | 653 | 11.529 | 0.333 | 22.842 | 1.00 | 9.53 |
| ATOM | 943 | ND1 | HIS | A | 653 | 9.881 | 1.740 | 22.569 | 1.00 | 10.01 |
| ATOM | 944 | CE1 | HIS | A | 653 | 10.440 | 1.559 | 21.387 | 1.00 | 6.46 |
| ATOM | 945 | NE2 | HIS | A | 653 | 11.444 | 0.708 | 21.526 | 1.00 | 6.65 |
| ATOM | 946 | C | HIS | A | 653 | 12.210 | 2.321 | 25.412 | 1.00 | 10.62 |
| ATOM | 947 | O | HIS | A | 653 | 13.024 | 1.459 | 25.741 | 1.00 | 8.86 |
| ATOM | 948 | N | ASP | A | 654 | 12.559 | 3.424 | 24.752 | 1.00 | 9.82 |
| ATOM | 949 | CA | ASP | A | 654 | 13.959 | 3.673 | 24.406 | 1.00 | 9.46 |
| ATOM | 950 | CB | ASP | A | 654 | 14.184 | 3.580 | 22.889 | 1.00 | 10.10 |
| ATOM | 951 | CG | ASP | A | 654 | 14.509 | 2.172 | 22.432 | 1.00 | 10.77 |
| ATOM | 952 | OD1 | ASP | A | 654 | 15.159 | 1.444 | 23.204 | 1.00 | 9.55 |
| ATOM | 953 | OD2 | ASP | A | 654 | 14.141 | 1.812 | 21.294 | 1.00 | 8.85 |
| ATOM | 954 | C | ASP | A | 654 | 14.477 | 5.024 | 24.896 | 1.00 | 9.92 |
| ATOM | 955 | O | ASP | A | 654 | 15.500 | 5.507 | 24.413 | 1.00 | 9.67 |
| ATOM | 956 | N | LEU | A | 655 | 13.783 | 5.619 | 25.865 | 1.00 | 9.19 |
| ATOM | 957 | CA | LEU | A | 655 | 14.169 | 6.922 | 26.408 | 1.00 | 10.06 |
| ATOM | 958 | CB | LEU | A | 655 | 13.364 | 7.214 | 27.677 | 1.00 | 8.55 |
| ATOM | 959 | CG | LEU | A | 655 | 11.900 | 7.603 | 27.479 | 1.00 | 10.24 |
| ATOM | 960 | CD1 | LEU | A | 655 | 11.151 | 7.468 | 28.805 | 1.00 | 9.10 |
| ATOM | 961 | CD2 | LEU | A | 655 | 11.819 | 9.028 | 26.944 | 1.00 | 15.71 |
| ATOM | 962 | C | LEU | A | 655 | 15.658 | 7.101 | 26.713 | 1.00 | 10.50 |
| ATOM | 963 | O | LEU | A | 655 | 16.279 | 6.268 | 27.372 | 1.00 | 9.92 |
| ATOM | 964 | N | ASP | A | 656 | 16.215 | 8.209 | 26.232 | 1.00 | 8.66 |
| ATOM | 965 | CA | ASP | A | 656 | 17.622 | 8.542 | 26.448 | 1.00 | 8.66 |
| ATOM | 966 | CB | ASP | A | 656 | 17.867 | 8.811 | 27.941 | 1.00 | 11.08 |
| ATOM | 967 | CG | ASP | A | 656 | 19.211 | 9.475 | 28.207 | 1.00 | 16.07 |
| ATOM | 968 | OD1 | ASP | A | 656 | 19.576 | 10.406 | 27.460 | 1.00 | 11.04 |
| ATOM | 969 | OD2 | ASP | A | 656 | 19.897 | 9.074 | 29.173 | 1.00 | 13.88 |
| ATOM | 970 | C | ASP | A | 656 | 18.600 | 7.483 | 25.939 | 1.00 | 8.59 |
| ATOM | 971 | O | ASP | A | 656 | 19.670 | 7.288 | 26.511 | 1.00 | 9.89 |
| ATOM | 972 | N | HIS | A | 657 | 18.242 | 6.806 | 24.851 | 1.00 | 9.07 |
| ATOM | 973 | CA | HIS | A | 657 | 19.118 | 5.790 | 24.284 | 1.00 | 10.63 |
| ATOM | 974 | CB | HIS | A | 657 | 18.412 | 5.068 | 23.137 | 1.00 | 9.06 |
| ATOM | 975 | CG | HIS | A | 657 | 19.095 | 3.810 | 22.712 | 1.00 | 8.47 |
| ATOM | 976 | CD2 | HIS | A | 657 | 20.347 | 3.589 | 22.245 | 1.00 | 7.97 |
| ATOM | 977 | ND1 | HIS | A | 657 | 18.480 | 2.575 | 22.748 | 1.00 | 12.46 |
| ATOM | 978 | CE1 | HIS | A | 657 | 19.322 | 1.652 | 22.322 | 1.00 | 5.38 |
| ATOM | 979 | NE2 | HIS | A | 657 | 20.464 | 2.242 | 22.011 | 1.00 | 12.33 |
| ATOM | 980 | C | HIS | A | 657 | 20.404 | 6.453 | 23.778 | 1.00 | 11.40 |
| ATOM | 981 | O | HIS | A | 657 | 20.362 | 7.404 | 22.993 | 1.00 | 11.45 |
| ATOM | 982 | N | PRO | A | 658 | 21.567 | 5.957 | 24.230 | 1.00 | 11.01 |
| ATOM | 983 | CD | PRO | A | 658 | 21.697 | 4.963 | 25.311 | 1.00 | 10.94 |
| ATOM | 984 | CA | PRO | A | 658 | 22.884 | 6.479 | 23.850 | 1.00 | 10.19 |
| ATOM | 985 | CB | PRO | A | 658 | 23.792 | 5.930 | 24.944 | 1.00 | 9.66 |
| ATOM | 986 | CG | PRO | A | 658 | 23.171 | 4.618 | 25.252 | 1.00 | 16.61 |
| ATOM | 987 | C | PRO | A | 658 | 23.386 | 6.123 | 22.451 | 1.00 | 11.02 |
| ATOM | 988 | O | PRO | A | 658 | 24.428 | 6.627 | 22.021 | 1.00 | 12.41 |
| ATOM | 989 | N | GLY | A | 659 | 22.659 | 5.265 | 21.744 | 1.00 | 8.82 |
| ATOM | 990 | CA | GLY | A | 659 | 23.080 | 4.888 | 20.405 | 1.00 | 8.78 |
| ATOM | 991 | C | GLY | A | 659 | 24.115 | 3.781 | 20.356 | 1.00 | 9.97 |
| ATOM | 992 | O | GLY | A | 659 | 24.750 | 3.566 | 19.316 | 1.00 | 12.32 |
| ATOM | 993 | N | VAL | A | 660 | 24.305 | 3.095 | 21.480 | 1.00 | 8.93 |
| ATOM | 994 | CA | VAL | A | 660 | 25.248 | 1.983 | 21.564 | 1.00 | 9.69 |
| ATOM | 995 | CB | VAL | A | 660 | 26.554 | 2.387 | 22.301 | 1.00 | 10.19 |
| ATOM | 996 | CG1 | VAL | A | 660 | 27.358 | 3.355 | 21.434 | 1.00 | 12.07 |
| ATOM | 997 | CG2 | VAL | A | 660 | 26.236 | 3.020 | 23.642 | 1.00 | 12.93 |
| ATOM | 998 | C | VAL | A | 660 | 24.545 | 0.834 | 22.289 | 1.00 | 10.76 |
| ATOM | 999 | O | VAL | A | 660 | 23.601 | 1.059 | 23.052 | 1.00 | 10.23 |
| ATOM | 1000 | N | SER | A | 661 | 25.002 | −0.392 | 22.049 | 1.00 | 7.63 |
| ATOM | 1001 | CA | SER | A | 661 | 24.371 | −1.577 | 22.624 | 1.00 | 10.33 |
| ATOM | 1002 | CB | SER | A | 661 | 24.748 | −2.801 | 21.793 | 1.00 | 8.76 |
| ATOM | 1003 | OG | SER | A | 661 | 26.099 | −3.151 | 22.021 | 1.00 | 9.56 |
| ATOM | 1004 | C | SER | A | 661 | 24.687 | −1.864 | 24.088 | 1.00 | 9.36 |
| ATOM | 1005 | O | SER | A | 661 | 25.549 | −1.230 | 24.696 | 1.00 | 10.42 |
| ATOM | 1006 | N | ASN | A | 662 | 23.969 | −2.838 | 24.643 | 1.00 | 10.10 |
| ATOM | 1007 | CA | ASN | A | 662 | 24.165 | −3.261 | 26.026 | 1.00 | 8.94 |
| ATOM | 1008 | CB | ASN | A | 662 | 23.177 | −4.369 | 26.384 | 1.00 | 12.24 |
| ATOM | 1009 | CG | ASN | A | 662 | 21.832 | −3.836 | 26.824 | 1.00 | 11.27 |
| ATOM | 1010 | OD1 | ASN | A | 662 | 20.794 | −4.457 | 26.592 | 1.00 | 19.20 |
| ATOM | 1011 | ND2 | ASN | A | 662 | 21.844 | −2.689 | 27.483 | 1.00 | 5.90 |
| ATOM | 1012 | C | ASN | A | 662 | 25.586 | −3.781 | 26.161 | 1.00 | 11.89 |
| ATOM | 1013 | O | ASN | A | 662 | 26.289 | −3.459 | 27.116 | 1.00 | 12.39 |
| ATOM | 1014 | N | GLN | A | 663 | 26.010 | −4.598 | 25.199 | 1.00 | 10.34 |
| ATOM | 1015 | CA | GLN | A | 663 | 27.356 | −5.139 | 25.246 | 1.00 | 11.06 |
| ATOM | 1016 | CB | GLN | A | 663 | 27.578 | −6.120 | 24.083 | 1.00 | 14.83 |
| ATOM | 1017 | CG | GLN | A | 663 | 26.677 | −7.373 | 24.197 | 1.00 | 25.28 |
| ATOM | 1018 | CD | GLN | A | 663 | 25.283 | −7.191 | 23.575 | 1.00 | 24.96 |
| ATOM | 1019 | OE1 | GLN | A | 663 | 24.919 | −6.113 | 23.106 | 1.00 | 31.10 |
| ATOM | 1020 | NE2 | GLN | A | 663 | 24.497 | −8.271 | 23.572 | 1.00 | 46.85 |
| ATOM | 1021 | C | GLN | A | 663 | 28.422 | −4.039 | 25.236 | 1.00 | 13.51 |
| ATOM | 1022 | O | GLN | A | 663 | 29.439 | −4.161 | 25.921 | 1.00 | 12.70 |
| ATOM | 1023 | N | PHE | A | 664 | 28.184 | −2.956 | 24.504 | 1.00 | 12.64 |
| ATOM | 1024 | CA | PHE | A | 664 | 29.145 | −1.852 | 24.465 | 1.00 | 11.20 |
| ATOM | 1025 | CB | PHE | A | 664 | 28.754 | −0.838 | 23.380 | 1.00 | 12.60 |
| ATOM | 1026 | CG | PHE | A | 664 | 29.669 | 0.355 | 23.296 | 1.00 | 15.21 |
| ATOM | 1027 | CD1 | PHE | A | 664 | 29.554 | 1.407 | 24.198 | 1.00 | 13.93 |
| ATOM | 1028 | CD2 | PHE | A | 664 | 30.668 | 0.409 | 22.336 | 1.00 | 10.34 |
| ATOM | 1029 | CE1 | PHE | A | 664 | 30.419 | 2.484 | 24.144 | 1.00 | 11.59 |
| ATOM | 1030 | CE2 | PHE | A | 664 | 31.541 | 1.487 | 22.275 | 1.00 | 16.42 |
| ATOM | 1031 | CZ | PHE | A | 664 | 31.418 | 2.527 | 23.182 | 1.00 | 18.27 |
| ATOM | 1032 | C | PHE | A | 664 | 29.207 | −1.171 | 25.830 | 1.00 | 11.75 |
| ATOM | 1033 | O | PHE | A | 664 | 30.290 | −0.818 | 26.306 | 1.00 | 11.28 |
| ATOM | 1034 | N | LEU | A | 665 | 28.046 | −0.980 | 26.452 | 1.00 | 11.17 |
| ATOM | 1035 | CA | LEU | A | 665 | 27.982 | −0.346 | 27.768 | 1.00 | 11.46 |
| ATOM | 1036 | CB | LEU | A | 665 | 26.524 | −0.163 | 28.204 | 1.00 | 11.38 |
| ATOM | 1037 | CG | LEU | A | 665 | 25.703 | 0.864 | 27.420 | 1.00 | 9.72 |
| ATOM | 1038 | CD1 | LEU | A | 665 | 24.304 | 0.959 | 28.015 | 1.00 | 16.35 |
| ATOM | 1039 | CD2 | LEU | A | 665 | 26.399 | 2.215 | 27.470 | 1.00 | 13.26 |
| ATOM | 1040 | C | LEU | A | 665 | 28.726 | −1.200 | 28.793 | 1.00 | 13.75 |
| ATOM | 1041 | O | LEU | A | 665 | 29.404 | −0.686 | 29.681 | 1.00 | 11.85 |
| ATOM | 1042 | N | ILE | A | 666 | 28.591 | −2.512 | 28.661 | 1.00 | 12.41 |
| ATOM | 1043 | CA | ILE | A | 666 | 29.259 | −3.444 | 29.559 | 1.00 | 13.52 |
| ATOM | 1044 | CB | ILE | A | 666 | 28.773 | −4.887 | 29.300 | 1.00 | 13.10 |
| ATOM | 1045 | CG2 | ILE | A | 666 | 29.667 | −5.882 | 30.020 | 1.00 | 13.00 |
| ATOM | 1046 | CG1 | ILE | A | 666 | 27.322 | −5.036 | 29.761 | 1.00 | 10.73 |
| ATOM | 1047 | CD1 | ILE | A | 666 | 26.687 | −6.346 | 29.369 | 1.00 | 14.67 |
| ATOM | 1048 | C | ILE | A | 666 | 30.770 | −3.381 | 29.356 | 1.00 | 11.71 |
| ATOM | 1049 | O | ILE | A | 666 | 31.541 | −3.297 | 30.317 | 1.00 | 14.40 |
| ATOM | 1050 | N | ASN | A | 667 | 31.183 | −3.408 | 28.094 | 1.00 | 12.52 |
| ATOM | 1051 | CA | ASN | A | 667 | 32.599 | −3.382 | 27.733 | 1.00 | 11.69 |
| ATOM | 1052 | CB | ASN | A | 667 | 32.745 | −3.652 | 26.232 | 1.00 | 15.94 |
| ATOM | 1053 | CG | ASN | A | 667 | 32.340 | −5.065 | 25.848 | 1.00 | 13.91 |
| ATOM | 1054 | OD1 | ASN | A | 667 | 32.138 | −5.363 | 24.669 | 1.00 | 17.63 |
| ATOM | 1055 | ND2 | ASN | A | 667 | 32.233 | −5.946 | 26.837 | 1.00 | 11.25 |
| ATOM | 1056 | C | ASN | A | 667 | 33.323 | −2.090 | 28.091 | 1.00 | 15.59 |
| ATOM | 1057 | O | ASN | A | 667 | 34.530 | −2.104 | 28.355 | 1.00 | 16.72 |
| ATOM | 1058 | N | THR | A | 668 | 32.601 | −0.974 | 28.098 | 1.00 | 15.05 |
| ATOM | 1059 | CA | THR | A | 668 | 33.211 | 0.308 | 28.428 | 1.00 | 16.29 |
| ATOM | 1060 | CB | THR | A | 668 | 32.670 | 1.432 | 27.520 | 1.00 | 16.12 |
| ATOM | 1061 | OG1 | THR | A | 668 | 31.237 | 1.448 | 27.567 | 1.00 | 19.49 |
| ATOM | 1062 | CG2 | THR | A | 668 | 33.133 | 1.207 | 26.078 | 1.00 | 15.93 |
| ATOM | 1063 | C | THR | A | 668 | 32.995 | 0.672 | 29.895 | 1.00 | 18.10 |
| ATOM | 1064 | O | THR | A | 668 | 33.158 | 1.830 | 30.297 | 1.00 | 20.65 |
| ATOM | 1065 | N | ASN | A | 669 | 32.617 | −0.330 | 30.682 | 1.00 | 17.75 |
| ATOM | 1066 | CA | ASN | A | 669 | 32.402 | −0.177 | 32.115 | 1.00 | 20.15 |
| ATOM | 1067 | CB | ASN | A | 669 | 33.749 | 0.053 | 32.797 | 1.00 | 28.92 |
| ATOM | 1068 | CG | ASN | A | 669 | 34.791 | −0.955 | 32.361 | 1.00 | 34.79 |
| ATOM | 1069 | OD1 | ASN | A | 669 | 34.619 | −2.160 | 32.543 | 1.00 | 40.75 |
| ATOM | 1070 | ND2 | ASN | A | 669 | 35.877 | −0.468 | 31.772 | 1.00 | 36.76 |
| ATOM | 1071 | C | ASN | A | 669 | 31.428 | 0.926 | 32.515 | 1.00 | 20.87 |
| ATOM | 1072 | O | ASN | A | 669 | 31.690 | 1.696 | 33.443 | 1.00 | 21.59 |
| ATOM | 1073 | N | SER | A | 670 | 30.302 | 0.996 | 31.819 | 1.00 | 17.34 |
| ATOM | 1074 | CA | SER | A | 670 | 29.286 | 1.992 | 32.120 | 1.00 | 19.60 |
| ATOM | 1075 | CB | SER | A | 670 | 28.103 | 1.836 | 31.160 | 1.00 | 18.96 |
| ATOM | 1076 | OG | SER | A | 670 | 27.023 | 2.672 | 31.535 | 1.00 | 22.69 |
| ATOM | 1077 | C | SER | A | 670 | 28.803 | 1.787 | 33.551 | 1.00 | 16.24 |
| ATOM | 1078 | O | SER | A | 670 | 28.755 | 0.655 | 34.041 | 1.00 | 14.46 |
| ATOM | 1079 | N | GLU | A | 671 | 28.455 | 2.879 | 34.227 | 1.00 | 18.45 |
| ATOM | 1080 | CA | GLU | A | 671 | 27.951 | 2.775 | 35.589 | 1.00 | 19.69 |
| ATOM | 1081 | CB | GLU | A | 671 | 27.756 | 4.154 | 36.209 | 1.00 | 17.48 |
| ATOM | 1082 | CG | GLU | A | 671 | 28.985 | 5.024 | 36.189 | 1.00 | 31.14 |
| ATOM | 1083 | CD | GLU | A | 671 | 28.901 | 6.156 | 37.191 | 1.00 | 32.78 |
| ATOM | 1084 | OE1 | GLU | A | 671 | 27.830 | 6.796 | 37.291 | 1.00 | 31.62 |
| ATOM | 1085 | OE2 | GLU | A | 671 | 29.914 | 6.406 | 37.875 | 1.00 | 26.52 |
| ATOM | 1086 | C | GLU | A | 671 | 26.611 | 2.058 | 35.549 | 1.00 | 18.77 |
| ATOM | 1087 | O | GLU | A | 671 | 26.253 | 1.332 | 36.482 | 1.00 | 19.75 |
| ATOM | 1088 | N | LEU | A | 672 | 25.868 | 2.281 | 34.466 | 1.00 | 17.92 |
| ATOM | 1089 | CA | LEU | A | 672 | 24.569 | 1.646 | 34.279 | 1.00 | 15.76 |
| ATOM | 1090 | CB | LEU | A | 672 | 23.954 | 2.067 | 32.942 | 1.00 | 12.50 |
| ATOM | 1091 | CG | LEU | A | 672 | 23.478 | 3.509 | 32.771 | 1.00 | 18.92 |
| ATOM | 1092 | CD1 | LEU | A | 672 | 23.101 | 3.741 | 31.314 | 1.00 | 18.65 |
| ATOM | 1093 | CD2 | LEU | A | 672 | 22.292 | 3.766 | 33.678 | 1.00 | 24.52 |
| ATOM | 1094 | C | LEU | A | 672 | 24.738 | 0.130 | 34.291 | 1.00 | 13.50 |
| ATOM | 1095 | O | LEU | A | 672 | 23.961 | −0.586 | 34.912 | 1.00 | 12.92 |
| ATOM | 1096 | N | ALA | A | 673 | 25.755 | −0.358 | 33.590 | 1.00 | 13.11 |
| ATOM | 1097 | CA | ALA | A | 673 | 26.001 | −1.792 | 33.546 | 1.00 | 13.53 |
| ATOM | 1098 | CB | ALA | A | 673 | 27.169 | −2.099 | 32.594 | 1.00 | 11.10 |
| ATOM | 1099 | C | ALA | A | 673 | 26.318 | −2.283 | 34.956 | 1.00 | 13.78 |
| ATOM | 1100 | O | ALA | A | 673 | 25.925 | −3.380 | 35.354 | 1.00 | 12.36 |
| ATOM | 1101 | N | LEU | A | 674 | 27.028 | −1.458 | 35.718 | 1.00 | 15.22 |
| ATOM | 1102 | CA | LEU | A | 674 | 27.394 | −1.829 | 37.076 | 1.00 | 16.65 |
| ATOM | 1103 | CB | LEU | A | 674 | 28.414 | −0.832 | 37.632 | 1.00 | 18.96 |
| ATOM | 1104 | CG | LEU | A | 674 | 29.225 | −1.323 | 38.832 | 1.00 | 26.52 |
| ATOM | 1105 | CD1 | LEU | A | 674 | 29.939 | −2.620 | 38.460 | 1.00 | 24.45 |
| ATOM | 1106 | CD2 | LEU | A | 674 | 30.231 | −0.258 | 39.246 | 1.00 | 20.57 |
| ATOM | 1107 | C | LEU | A | 674 | 26.152 | −1.866 | 37.960 | 1.00 | 15.94 |
| ATOM | 1108 | O | LEU | A | 674 | 25.930 | −2.818 | 38.709 | 1.00 | 16.86 |
| ATOM | 1109 | N | MET | A | 675 | 25.335 | −0.822 | 37.851 | 1.00 | 19.35 |
| ATOM | 1110 | CA | MET | A | 675 | 24.107 | −0.694 | 38.627 | 1.00 | 18.57 |
| ATOM | 1111 | CB | MET | A | 675 | 23.413 | 0.641 | 38.312 | 1.00 | 29.02 |
| ATOM | 1112 | CG | MET | A | 675 | 24.180 | 1.882 | 38.753 | 1.00 | 32.86 |
| ATOM | 1113 | SD | MET | A | 675 | 23.766 | 3.373 | 37.785 | 1.00 | 38.23 |
| ATOM | 1114 | CE | MET | A | 675 | 22.129 | 3.769 | 38.381 | 1.00 | 40.96 |
| ATOM | 1115 | C | MET | A | 675 | 23.133 | −1.830 | 38.346 | 1.00 | 17.83 |
| ATOM | 1116 | O | MET | A | 675 | 22.529 | −2.383 | 39.265 | 1.00 | 17.84 |
| ATOM | 1117 | N | TYR | A | 676 | 23.000 | −2.184 | 37.073 | 1.00 | 15.75 |
| ATOM | 1118 | CA | TYR | A | 676 | 22.052 | −3.212 | 36.679 | 1.00 | 16.85 |
| ATOM | 1119 | CB | TYR | A | 676 | 21.293 | −2.720 | 35.442 | 1.00 | 16.19 |
| ATOM | 1120 | CG | TYR | A | 676 | 20.619 | −1.374 | 35.668 | 1.00 | 20.30 |
| ATOM | 1121 | CD1 | TYR | A | 676 | 19.694 | −1.197 | 36.691 | 1.00 | 31.51 |
| ATOM | 1122 | CE1 | TYR | A | 676 | 19.107 | 0.045 | 36.922 | 1.00 | 18.89 |
| ATOM | 1123 | CD2 | TYR | A | 676 | 20.934 | −0.276 | 34.877 | 1.00 | 23.78 |
| ATOM | 1124 | CE2 | TYR | A | 676 | 20.354 | 0.962 | 35.097 | 1.00 | 27.90 |
| ATOM | 1125 | CZ | TYR | A | 676 | 19.443 | 1.117 | 36.118 | 1.00 | 24.78 |
| ATOM | 1126 | OH | TYR | A | 676 | 18.866 | 2.354 | 36.326 | 1.00 | 28.03 |
| ATOM | 1127 | C | TYR | A | 676 | 22.605 | −4.623 | 36.471 | 1.00 | 16.48 |
| ATOM | 1128 | O | TYR | A | 676 | 21.949 | −5.478 | 35.872 | 1.00 | 15.88 |
| ATOM | 1129 | N | ASN | A | 677 | 23.814 | −4.863 | 36.968 | 1.00 | 16.14 |
| ATOM | 1130 | CA | ASN | A | 677 | 24.433 | −6.182 | 36.888 | 1.00 | 14.23 |
| ATOM | 1131 | CB | ASN | A | 677 | 23.666 | −7.138 | 37.812 | 1.00 | 21.33 |
| ATOM | 1132 | CG | ASN | A | 677 | 24.382 | −8.452 | 38.022 | 1.00 | 29.37 |
| ATOM | 1133 | OD1 | ASN | A | 677 | 25.570 | −8.478 | 38.349 | 1.00 | 38.44 |
| ATOM | 1134 | ND2 | ASN | A | 677 | 23.662 | −9.555 | 37.847 | 1.00 | 42.62 |
| ATOM | 1135 | C | ASN | A | 677 | 24.516 | −6.765 | 35.474 | 1.00 | 15.05 |
| ATOM | 1136 | O | ASN | A | 677 | 24.332 | −7.966 | 35.275 | 1.00 | 14.59 |
| ATOM | 1137 | N | ASP | A | 678 | 24.802 | −5.904 | 34.502 | 1.00 | 13.69 |
| ATOM | 1138 | CA | ASP | A | 678 | 24.926 | −6.302 | 33.100 | 1.00 | 16.08 |
| ATOM | 1139 | CB | ASP | A | 678 | 26.080 | −7.293 | 32.915 | 1.00 | 12.96 |
| ATOM | 1140 | CG | ASP | A | 678 | 27.430 | −6.691 | 33.234 | 1.00 | 18.27 |
| ATOM | 1141 | OD1 | ASP | A | 678 | 27.519 | −5.452 | 33.377 | 1.00 | 16.21 |
| ATOM | 1142 | OD2 | ASP | A | 678 | 28.406 | −7.464 | 33.334 | 1.00 | 20.95 |
| ATOM | 1143 | C | ASP | A | 678 | 23.667 | −6.919 | 32.499 | 1.00 | 16.35 |
| ATOM | 1144 | O | ASP | A | 678 | 23.731 | −7.509 | 31.420 | 1.00 | 17.49 |
| ATOM | 1145 | N | GLU | A | 679 | 22.534 | −6.790 | 33.184 | 1.00 | 14.03 |
| ATOM | 1146 | CA | GLU | A | 679 | 21.278 | −7.358 | 32.690 | 1.00 | 14.39 |
| ATOM | 1147 | CB | GLU | A | 679 | 20.567 | −8.133 | 33.799 | 1.00 | 14.04 |
| ATOM | 1148 | CG | GLU | A | 679 | 21.314 | −9.351 | 34.300 | 1.00 | 22.79 |
| ATOM | 1149 | CD | GLU | A | 679 | 20.514 | −10.128 | 35.330 | 1.00 | 43.42 |
| ATOM | 1150 | OE1 | GLU | A | 679 | 19.464 | −10.699 | 34.964 | 1.00 | 51.30 |
| ATOM | 1151 | OE2 | GLU | A | 679 | 20.931 | −10.162 | 36.507 | 1.00 | 41.34 |
| ATOM | 1152 | C | GLU | A | 679 | 20.320 | −6.301 | 32.151 | 1.00 | 12.89 |
| ATOM | 1153 | O | GLU | A | 679 | 19.873 | −5.430 | 32.895 | 1.00 | 13.61 |
| ATOM | 1154 | N | SER | A | 680 | 19.993 | −6.401 | 30.863 | 1.00 | 10.87 |
| ATOM | 1155 | CA | SER | A | 680 | 19.086 | −5.456 | 30.209 | 1.00 | 12.50 |
| ATOM | 1156 | CB | SER | A | 680 | 17.627 | −5.787 | 30.555 | 1.00 | 12.08 |
| ATOM | 1157 | OG | SER | A | 680 | 17.267 | −7.087 | 30.111 | 1.00 | 11.26 |
| ATOM | 1158 | C | SER | A | 680 | 19.409 | −4.038 | 30.663 | 1.00 | 11.78 |
| ATOM | 1159 | O | SER | A | 680 | 18.517 | −3.280 | 31.045 | 1.00 | 12.76 |
| ATOM | 1160 | N | VAL | A | 681 | 20.693 | −3.693 | 30.617 | 1.00 | 9.67 |
| ATOM | 1161 | CA | VAL | A | 681 | 21.183 | −2.386 | 31.049 | 1.00 | 7.19 |
| ATOM | 1162 | CB | VAL | A | 681 | 22.688 | −2.248 | 30.725 | 1.00 | 11.97 |
| ATOM | 1163 | CG1 | VAL | A | 681 | 23.219 | −0.917 | 31.245 | 1.00 | 10.98 |
| ATOM | 1164 | CG2 | VAL | A | 681 | 23.460 | −3.423 | 31.346 | 1.00 | 12.93 |
| ATOM | 1165 | C | VAL | A | 681 | 20.426 | −1.195 | 30.453 | 1.00 | 9.58 |
| ATOM | 1166 | O | VAL | A | 681 | 19.870 | −0.378 | 31.185 | 1.00 | 10.02 |
| ATOM | 1167 | N | LEU | A | 681A | 20.420 | −1.094 | 29.128 | 1.00 | 9.51 |
| ATOM | 1168 | CA | LEU | A | 681A | 19.718 | −0.011 | 28.443 | 1.00 | 10.16 |
| ATOM | 1169 | CB | LEU | A | 681A | 19.773 | −0.216 | 26.929 | 1.00 | 7.24 |
| ATOM | 1170 | CG | LEU | A | 681A | 21.079 | 0.035 | 26.178 | 1.00 | 9.74 |
| ATOM | 1171 | CD1 | LEU | A | 681A | 20.979 | −0.563 | 24.788 | 1.00 | 13.02 |
| ATOM | 1172 | CD2 | LEU | A | 681A | 21.345 | 1.526 | 26.102 | 1.00 | 15.84 |
| ATOM | 1173 | C | LEU | A | 681A | 18.253 | 0.049 | 28.851 | 1.00 | 8.34 |
| ATOM | 1174 | O | LEU | A | 681A | 17.725 | 1.118 | 29.174 | 1.00 | 10.62 |
| ATOM | 1175 | N | GLU | A | 682 | 17.599 | −1.110 | 28.834 | 1.00 | 10.08 |
| ATOM | 1176 | CA | GLU | A | 682 | 16.183 | −1.191 | 29.161 | 1.00 | 7.57 |
| ATOM | 1177 | CB | GLU | A | 682 | 15.667 | −2.599 | 28.855 | 1.00 | 6.60 |
| ATOM | 1178 | CG | GLU | A | 682 | 15.731 | −2.954 | 27.348 | 1.00 | 8.78 |
| ATOM | 1179 | CD | GLU | A | 682 | 17.151 | −3.199 | 26.835 | 1.00 | 12.12 |
| ATOM | 1180 | OE1 | GLU | A | 682 | 18.001 | −3.693 | 27.614 | 1.00 | 10.38 |
| ATOM | 1181 | OE2 | GLU | A | 682 | 17.418 | −2.919 | 25.646 | 1.00 | 10.27 |
| ATOM | 1182 | C | GLU | A | 682 | 15.867 | −0.773 | 30.594 | 1.00 | 8.81 |
| ATOM | 1183 | O | GLU | A | 682 | 14.829 | −0.158 | 30.854 | 1.00 | 7.94 |
| ATOM | 1184 | N | HIS | A | 683 | 16.752 | −1.099 | 31.526 | 1.00 | 9.05 |
| ATOM | 1185 | CA | HIS | A | 683 | 16.531 | −0.677 | 32.897 | 1.00 | 10.40 |
| ATOM | 1186 | CB | HIS | A | 683 | 17.561 | −1.324 | 33.830 | 1.00 | 11.92 |
| ATOM | 1187 | CG | HIS | A | 683 | 17.161 | −2.686 | 34.296 | 1.00 | 10.38 |
| ATOM | 1188 | CD2 | HIS | A | 683 | 17.557 | −3.920 | 33.903 | 1.00 | 9.93 |
| ATOM | 1189 | ND1 | HIS | A | 683 | 16.172 | −2.888 | 35.238 | 1.00 | 14.33 |
| ATOM | 1190 | CE1 | HIS | A | 683 | 15.977 | −4.183 | 35.400 | 1.00 | 7.54 |
| ATOM | 1191 | NE2 | HIS | A | 683 | 16.806 | −4.831 | 34.599 | 1.00 | 15.67 |
| ATOM | 1192 | C | HIS | A | 683 | 16.631 | 0.846 | 32.925 | 1.00 | 11.08 |
| ATOM | 1193 | O | HIS | A | 683 | 15.868 | 1.512 | 33.621 | 1.00 | 10.48 |
| ATOM | 1194 | N | HIS | A | 684 | 17.553 | 1.397 | 32.138 | 1.00 | 10.86 |
| ATOM | 1195 | CA | HIS | A | 684 | 17.710 | 2.844 | 32.076 | 1.00 | 9.98 |
| ATOM | 1196 | CB | HIS | A | 684 | 18.941 | 3.233 | 31.256 | 1.00 | 9.09 |
| ATOM | 1197 | CG | HIS | A | 684 | 19.229 | 4.697 | 31.284 | 1.00 | 8.54 |
| ATOM | 1198 | CD2 | HIS | A | 684 | 19.399 | 5.555 | 32.322 | 1.00 | 5.20 |
| ATOM | 1199 | ND1 | HIS | A | 684 | 19.313 | 5.467 | 30.142 | 1.00 | 17.15 |
| ATOM | 1200 | CE1 | HIS | A | 684 | 19.518 | 6.728 | 30.474 | 1.00 | 9.30 |
| ATOM | 1201 | NE2 | HIS | A | 684 | 19.573 | 6.807 | 31.795 | 1.00 | 15.79 |
| ATOM | 1202 | C | HIS | A | 684 | 16.473 | 3.502 | 31.465 | 1.00 | 9.44 |
| ATOM | 1203 | O | HIS | A | 684 | 16.020 | 4.542 | 31.941 | 1.00 | 11.75 |
| ATOM | 1204 | N | HIS | A | 685 | 15.929 | 2.898 | 30.411 | 1.00 | 9.42 |
| ATOM | 1205 | CA | HIS | A | 685 | 14.740 | 3.447 | 29.757 | 1.00 | 7.57 |
| ATOM | 1206 | CB | HIS | A | 685 | 14.361 | 2.621 | 28.521 | 1.00 | 14.86 |
| ATOM | 1207 | CG | HIS | A | 685 | 15.472 | 2.468 | 27.521 | 1.00 | 7.24 |
| ATOM | 1208 | CD2 | HIS | A | 685 | 15.846 | 1.402 | 26.773 | 1.00 | 9.32 |
| ATOM | 1209 | ND1 | HIS | A | 685 | 16.299 | 3.506 | 27.161 | 1.00 | 8.86 |
| ATOM | 1210 | CE1 | HIS | A | 685 | 17.141 | 3.088 | 26.227 | 1.00 | 14.03 |
| ATOM | 1211 | NE2 | HIS | A | 685 | 16.886 | 1.819 | 25.974 | 1.00 | 8.62 |
| ATOM | 1212 | C | HIS | A | 685 | 13.574 | 3.446 | 30.747 | 1.00 | 9.27 |
| ATOM | 1213 | O | HIS | A | 685 | 12.798 | 4.401 | 30.805 | 1.00 | 9.78 |
| ATOM | 1214 | N | PHE | A | 686 | 13.439 | 2.376 | 31.523 | 1.00 | 8.10 |
| ATOM | 1215 | CA | PHE | A | 686 | 12.351 | 2.314 | 32.507 | 1.00 | 10.05 |
| ATOM | 1216 | CB | PHE | A | 686 | 12.291 | 0.923 | 33.151 | 1.00 | 8.82 |
| ATOM | 1217 | CG | PHE | A | 686 | 11.281 | 0.808 | 34.263 | 1.00 | 10.55 |
| ATOM | 1218 | CD1 | PHE | A | 686 | 9.937 | 1.078 | 34.035 | 1.00 | 10.53 |
| ATOM | 1219 | CD2 | PHE | A | 686 | 11.674 | 0.442 | 35.538 | 1.00 | 11.42 |
| ATOM | 1220 | CE1 | PHE | A | 686 | 9.013 | 0.985 | 35.061 | 1.00 | 14.12 |
| ATOM | 1221 | CE2 | PHE | A | 686 | 10.754 | 0.348 | 36.568 | 1.00 | 15.20 |
| ATOM | 1222 | CZ | PHE | A | 686 | 9.423 | 0.618 | 36.328 | 1.00 | 13.66 |
| ATOM | 1223 | C | PHE | A | 686 | 12.554 | 3.391 | 33.570 | 1.00 | 9.59 |
| ATOM | 1224 | O | PHE | A | 686 | 11.599 | 4.036 | 34.022 | 1.00 | 11.10 |
| ATOM | 1225 | N | ASP | A | 687 | 13.807 | 3.603 | 33.965 | 1.00 | 10.38 |
| ATOM | 1226 | CA | ASP | A | 687 | 14.169 | 4.620 | 34.950 | 1.00 | 13.63 |
| ATOM | 1227 | CB | ASP | A | 687 | 15.699 | 4.646 | 35.147 | 1.00 | 15.63 |
| ATOM | 1228 | CG | ASP | A | 687 | 16.163 | 5.810 | 36.032 | 1.00 | 35.43 |
| ATOM | 1229 | OD1 | ASP | A | 687 | 15.842 | 5.812 | 37.238 | 1.00 | 39.21 |
| ATOM | 1230 | OD2 | ASP | A | 687 | 16.855 | 6.721 | 35.514 | 1.00 | 46.67 |
| ATOM | 1231 | C | ASP | A | 687 | 13.701 | 5.983 | 34.453 | 1.00 | 11.19 |
| ATOM | 1232 | O | ASP | A | 687 | 13.079 | 6.742 | 35.191 | 1.00 | 12.63 |
| ATOM | 1233 | N | GLN | A | 688 | 14.026 | 6.266 | 33.194 | 1.00 | 10.97 |
| ATOM | 1234 | CA | GLN | A | 688 | 13.664 | 7.531 | 32.571 | 1.00 | 10.87 |
| ATOM | 1235 | CB | GLN | A | 688 | 14.331 | 7.623 | 31.193 | 1.00 | 15.23 |
| ATOM | 1236 | CG | GLN | A | 688 | 15.844 | 7.876 | 31.260 | 1.00 | 11.96 |
| ATOM | 1237 | CD | GLN | A | 688 | 16.179 | 9.350 | 31.456 | 1.00 | 14.39 |
| ATOM | 1238 | OE1 | GLN | A | 688 | 15.805 | 10.193 | 30.640 | 1.00 | 20.66 |
| ATOM | 1239 | NE2 | GLN | A | 688 | 16.881 | 9.663 | 32.535 | 1.00 | 29.50 |
| ATOM | 1240 | C | GLN | A | 688 | 12.158 | 7.651 | 32.455 | 1.00 | 10.36 |
| ATOM | 1241 | O | GLN | A | 688 | 11.587 | 8.729 | 32.641 | 1.00 | 10.53 |
| ATOM | 1242 | N | CYS | A | 689 | 11.495 | 6.536 | 32.160 | 1.00 | 8.48 |
| ATOM | 1243 | CA | CYS | A | 689 | 10.043 | 6.517 | 32.040 | 1.00 | 9.59 |
| ATOM | 1244 | CB | CYS | A | 689 | 9.558 | 5.097 | 31.706 | 1.00 | 11.54 |
| ATOM | 1245 | SG | CYS | A | 689 | 7.772 | 4.940 | 31.573 | 1.00 | 11.98 |
| ATOM | 1246 | C | CYS | A | 689 | 9.406 | 7.005 | 33.343 | 1.00 | 10.54 |
| ATOM | 1247 | O | CYS | A | 689 | 8.559 | 7.905 | 33.331 | 1.00 | 10.86 |
| ATOM | 1248 | N | LEU | A | 690 | 9.822 | 6.417 | 34.463 | 1.00 | 12.43 |
| ATOM | 1249 | CA | LEU | A | 690 | 9.316 | 6.786 | 35.787 | 1.00 | 13.70 |
| ATOM | 1250 | CB | LEU | A | 690 | 9.968 | 5.912 | 36.863 | 1.00 | 15.35 |
| ATOM | 1251 | CG | LEU | A | 690 | 9.620 | 4.423 | 36.853 | 1.00 | 18.35 |
| ATOM | 1252 | CD1 | LEU | A | 690 | 10.326 | 3.723 | 38.008 | 1.00 | 25.25 |
| ATOM | 1253 | CD2 | LEU | A | 690 | 8.118 | 4.248 | 36.969 | 1.00 | 17.06 |
| ATOM | 1254 | C | LEU | A | 690 | 9.605 | 8.247 | 36.093 | 1.00 | 14.05 |
| ATOM | 1255 | O | LEU | A | 690 | 8.767 | 8.975 | 36.633 | 1.00 | 15.63 |
| ATOM | 1256 | N | MET | A | 691 | 10.817 | 8.681 | 35.750 | 1.00 | 13.87 |
| ATOM | 1257 | CA | MET | A | 691 | 11.256 | 10.042 | 35.975 | 1.00 | 14.41 |
| ATOM | 1258 | CB | MET | A | 691 | 12.676 | 10.212 | 35.411 | 1.00 | 14.62 |
| ATOM | 1259 | CG | MET | A | 691 | 13.371 | 11.496 | 35.757 | 1.00 | 32.36 |
| ATOM | 1260 | SD | MET | A | 691 | 12.902 | 12.755 | 34.635 | 1.00 | 45.55 |
| ATOM | 1261 | CE | MET | A | 691 | 13.918 | 12.471 | 33.200 | 1.00 | 37.72 |
| ATOM | 1262 | C | MET | A | 691 | 10.285 | 11.024 | 35.336 | 1.00 | 13.72 |
| ATOM | 1263 | O | MET | A | 691 | 9.863 | 11.990 | 35.971 | 1.00 | 14.45 |
| ATOM | 1264 | N | ILE | A | 692 | 9.903 | 10.758 | 34.091 | 1.00 | 10.48 |
| ATOM | 1265 | CA | ILE | A | 692 | 8.980 | 11.644 | 33.400 | 1.00 | 9.98 |
| ATOM | 1266 | CB | ILE | A | 692 | 8.966 | 11.371 | 31.895 | 1.00 | 10.00 |
| ATOM | 1267 | CG2 | ILE | A | 692 | 7.909 | 12.240 | 31.224 | 1.00 | 12.51 |
| ATOM | 1268 | CG1 | ILE | A | 692 | 10.346 | 11.691 | 31.315 | 1.00 | 14.40 |
| ATOM | 1269 | CD1 | ILE | A | 692 | 10.482 | 11.400 | 29.841 | 1.00 | 19.81 |
| ATOM | 1270 | C | ILE | A | 692 | 7.578 | 11.534 | 33.973 | 1.00 | 10.09 |
| ATOM | 1271 | O | ILE | A | 692 | 6.906 | 12.547 | 34.166 | 1.00 | 11.41 |
| ATOM | 1272 | N | LEU | A | 693 | 7.149 | 10.316 | 34.288 | 1.00 | 12.13 |
| ATOM | 1273 | CA | LEU | A | 693 | 5.821 | 10.111 | 34.862 | 1.00 | 13.85 |
| ATOM | 1274 | CB | LEU | A | 693 | 5.548 | 8.615 | 35.058 | 1.00 | 13.96 |
| ATOM | 1275 | CG | LEU | A | 693 | 5.212 | 7.826 | 33.789 | 1.00 | 10.33 |
| ATOM | 1276 | CD1 | LEU | A | 693 | 5.183 | 6.335 | 34.082 | 1.00 | 17.28 |
| ATOM | 1277 | CD2 | LEU | A | 693 | 3.868 | 8.305 | 33.243 | 1.00 | 9.40 |
| ATOM | 1278 | C | LEU | A | 693 | 5.644 | 10.837 | 36.193 | 1.00 | 14.19 |
| ATOM | 1279 | O | LEU | A | 693 | 4.524 | 11.179 | 36.577 | 1.00 | 16.64 |
| ATOM | 1280 | N | ASN | A | 694 | 6.747 | 11.073 | 36.893 | 1.00 | 16.46 |
| ATOM | 1281 | CA | ASN | A | 694 | 6.697 | 11.755 | 38.183 | 1.00 | 16.63 |
| ATOM | 1282 | CB | ASN | A | 694 | 7.686 | 11.116 | 39.159 | 1.00 | 20.12 |
| ATOM | 1283 | CG | ASN | A | 694 | 7.231 | 9.760 | 39.641 | 1.00 | 24.99 |
| ATOM | 1284 | OD1 | ASN | A | 694 | 7.869 | 8.742 | 39.373 | 1.00 | 30.33 |
| ATOM | 1285 | ND2 | ASN | A | 694 | 6.115 | 9.736 | 40.360 | 1.00 | 25.93 |
| ATOM | 1286 | C | ASN | A | 694 | 6.989 | 13.247 | 38.101 | 1.00 | 18.31 |
| ATOM | 1287 | O | ASN | A | 694 | 6.855 | 13.958 | 39.093 | 1.00 | 18.26 |
| ATOM | 1288 | N | SER | A | 695 | 7.395 | 13.721 | 36.930 | 1.00 | 16.05 |
| ATOM | 1289 | CA | SER | A | 695 | 7.704 | 15.139 | 36.755 | 1.00 | 17.70 |
| ATOM | 1290 | CB | SER | A | 695 | 8.321 | 15.376 | 35.376 | 1.00 | 19.30 |
| ATOM | 1291 | OG | SER | A | 695 | 9.607 | 14.793 | 35.298 | 1.00 | 34.04 |
| ATOM | 1292 | C | SER | A | 695 | 6.471 | 16.022 | 36.914 | 1.00 | 15.83 |
| ATOM | 1293 | O | SER | A | 695 | 5.400 | 15.710 | 36.406 | 1.00 | 17.46 |
| ATOM | 1294 | N | PRO | A | 696 | 6.611 | 17.144 | 37.630 | 1.00 | 18.97 |
| ATOM | 1295 | CD | PRO | A | 696 | 7.779 | 17.577 | 38.415 | 1.00 | 26.97 |
| ATOM | 1296 | CA | PRO | A | 696 | 5.480 | 18.053 | 37.830 | 1.00 | 18.99 |
| ATOM | 1297 | CB | PRO | A | 696 | 6.113 | 19.220 | 38.579 | 1.00 | 22.14 |
| ATOM | 1298 | CG | PRO | A | 696 | 7.154 | 18.534 | 39.414 | 1.00 | 28.71 |
| ATOM | 1299 | C | PRO | A | 696 | 4.866 | 18.480 | 36.498 | 1.00 | 16.02 |
| ATOM | 1300 | O | PRO | A | 696 | 5.585 | 18.839 | 35.564 | 1.00 | 18.08 |
| ATOM | 1301 | N | GLY | A | 697 | 3.539 | 18.425 | 36.418 | 1.00 | 16.17 |
| ATOM | 1302 | CA | GLY | A | 697 | 2.843 | 18.804 | 35.201 | 1.00 | 16.65 |
| ATOM | 1303 | C | GLY | A | 697 | 2.862 | 17.725 | 34.130 | 1.00 | 14.72 |
| ATOM | 1304 | O | GLY | A | 697 | 2.283 | 17.901 | 33.060 | 1.00 | 15.86 |
| ATOM | 1305 | N | ASN | A | 698 | 3.511 | 16.601 | 34.424 | 1.00 | 14.68 |
| ATOM | 1306 | CA | ASN | A | 698 | 3.621 | 15.493 | 33.471 | 1.00 | 14.66 |
| ATOM | 1307 | CB | ASN | A | 698 | 5.099 | 15.209 | 33.174 | 1.00 | 15.25 |
| ATOM | 1308 | CG | ASN | A | 698 | 5.760 | 16.304 | 32.365 | 1.00 | 19.94 |
| ATOM | 1309 | OD1 | ASN | A | 698 | 5.883 | 16.199 | 31.146 | 1.00 | 13.89 |
| ATOM | 1310 | ND2 | ASN | A | 698 | 6.186 | 17.367 | 33.039 | 1.00 | 16.33 |
| ATOM | 1311 | C | ASN | A | 698 | 2.990 | 14.198 | 33.973 | 1.00 | 14.82 |
| ATOM | 1312 | O | ASN | A | 698 | 3.044 | 13.174 | 33.293 | 1.00 | 13.20 |
| ATOM | 1313 | N | GLN | A | 699 | 2.379 | 14.243 | 35.151 | 1.00 | 13.46 |
| ATOM | 1314 | CA | GLN | A | 699 | 1.807 | 13.041 | 35.749 | 1.00 | 14.53 |
| ATOM | 1315 | CB | GLN | A | 699 | 1.652 | 13.281 | 37.251 | 1.00 | 16.25 |
| ATOM | 1316 | CG | GLN | A | 699 | 2.957 | 13.713 | 37.902 | 1.00 | 17.08 |
| ATOM | 1317 | CD | GLN | A | 699 | 2.787 | 14.103 | 39.350 | 1.00 | 37.15 |
| ATOM | 1318 | OE1 | GLN | A | 699 | 2.147 | 15.105 | 39.661 | 1.00 | 41.13 |
| ATOM | 1319 | NE2 | GLN | A | 699 | 3.357 | 13.307 | 40.248 | 1.00 | 41.91 |
| ATOM | 1320 | C | GLN | A | 699 | 0.499 | 12.522 | 35.158 | 1.00 | 13.96 |
| ATOM | 1321 | O | GLN | A | 699 | −0.567 | 12.676 | 35.754 | 1.00 | 15.67 |
| ATOM | 1322 | N | ILE | A | 700 | 0.582 | 11.867 | 34.003 | 1.00 | 13.44 |
| ATOM | 1323 | CA | ILE | A | 700 | −0.617 | 11.348 | 33.356 | 1.00 | 14.25 |
| ATOM | 1324 | CB | ILE | A | 700 | −0.345 | 10.974 | 31.881 | 1.00 | 15.57 |
| ATOM | 1325 | CG2 | ILE | A | 700 | 0.010 | 12.222 | 31.086 | 1.00 | 15.78 |
| ATOM | 1326 | CG1 | ILE | A | 700 | 0.777 | 9.941 | 31.793 | 1.00 | 16.74 |
| ATOM | 1327 | CD1 | ILE | A | 700 | 0.940 | 9.361 | 30.407 | 1.00 | 12.98 |
| ATOM | 1328 | C | ILE | A | 700 | −1.262 | 10.151 | 34.064 | 1.00 | 13.88 |
| ATOM | 1329 | O | ILE | A | 700 | −2.342 | 9.709 | 33.673 | 1.00 | 14.53 |
| ATOM | 1330 | N | LEU | A | 701 | −0.609 | 9.624 | 35.098 | 1.00 | 11.60 |
| ATOM | 1331 | CA | LEU | A | 701 | −1.169 | 8.495 | 35.845 | 1.00 | 14.58 |
| ATOM | 1332 | CB | LEU | A | 701 | −0.150 | 7.358 | 35.965 | 1.00 | 13.85 |
| ATOM | 1333 | CG | LEU | A | 701 | 0.310 | 6.673 | 34.677 | 1.00 | 11.07 |
| ATOM | 1334 | CD1 | LEU | A | 701 | 1.270 | 5.539 | 35.031 | 1.00 | 13.34 |
| ATOM | 1335 | CD2 | LEU | A | 701 | −0.895 | 6.128 | 33.921 | 1.00 | 12.89 |
| ATOM | 1336 | C | LEU | A | 701 | −1.612 | 8.914 | 37.247 | 1.00 | 13.75 |
| ATOM | 1337 | O | LEU | A | 701 | −2.053 | 8.084 | 38.040 | 1.00 | 15.74 |
| ATOM | 1338 | N | SER | A | 702 | −1.497 | 10.201 | 37.547 | 1.00 | 15.74 |
| ATOM | 1339 | CA | SER | A | 702 | −1.872 | 10.717 | 38.860 | 1.00 | 18.36 |
| ATOM | 1340 | CB | SER | A | 702 | −1.573 | 12.217 | 38.949 | 1.00 | 20.19 |
| ATOM | 1341 | OG | SER | A | 702 | −2.446 | 12.958 | 38.113 | 1.00 | 21.94 |
| ATOM | 1342 | C | SER | A | 702 | −3.347 | 10.490 | 39.176 | 1.00 | 19.34 |
| ATOM | 1343 | O | SER | A | 702 | −3.754 | 10.559 | 40.336 | 1.00 | 20.75 |
| ATOM | 1344 | N | GLY | A | 703 | −4.144 | 10.224 | 38.148 | 1.00 | 18.92 |
| ATOM | 1345 | CA | GLY | A | 703 | −5.565 | 10.012 | 38.359 | 1.00 | 20.40 |
| ATOM | 1346 | C | GLY | A | 703 | −5.944 | 8.600 | 38.764 | 1.00 | 21.19 |
| ATOM | 1347 | O | GLY | A | 703 | −7.036 | 8.369 | 39.284 | 1.00 | 20.43 |
| ATOM | 1348 | N | LEU | A | 704 | −5.045 | 7.649 | 38.539 | 1.00 | 16.00 |
| ATOM | 1349 | CA | LEU | A | 704 | −5.326 | 6.259 | 38.876 | 1.00 | 15.59 |
| ATOM | 1350 | CB | LEU | A | 704 | −4.262 | 5.335 | 38.278 | 1.00 | 9.29 |
| ATOM | 1351 | CG | LEU | A | 704 | −4.459 | 4.921 | 36.823 | 1.00 | 14.10 |
| ATOM | 1352 | CD1 | LEU | A | 704 | −4.510 | 6.143 | 35.926 | 1.00 | 17.73 |
| ATOM | 1353 | CD2 | LEU | A | 704 | −3.312 | 3.998 | 36.418 | 1.00 | 11.19 |
| ATOM | 1354 | C | LEU | A | 704 | −5.419 | 5.993 | 40.368 | 1.00 | 16.40 |
| ATOM | 1355 | O | LEU | A | 704 | −4.773 | 6.659 | 41.178 | 1.00 | 16.88 |
| ATOM | 1356 | N | SER | A | 705 | −6.233 | 5.004 | 40.721 | 1.00 | 15.43 |
| ATOM | 1357 | CA | SER | A | 705 | −6.395 | 4.621 | 42.111 | 1.00 | 15.96 |
| ATOM | 1358 | CB | SER | A | 705 | −7.556 | 3.640 | 42.268 | 1.00 | 15.57 |
| ATOM | 1359 | OG | SER | A | 705 | −7.242 | 2.402 | 41.658 | 1.00 | 15.44 |
| ATOM | 1360 | C | SER | A | 705 | −5.092 | 3.947 | 42.515 | 1.00 | 16.32 |
| ATOM | 1361 | O | SER | A | 705 | −4.280 | 3.583 | 41.662 | 1.00 | 17.25 |
| ATOM | 1362 | N | ILE | A | 706 | −4.886 | 3.782 | 43.812 | 1.00 | 17.56 |
| ATOM | 1363 | CA | ILE | A | 706 | −3.666 | 3.164 | 44.292 | 1.00 | 16.88 |
| ATOM | 1364 | CB | ILE | A | 706 | −3.679 | 3.086 | 45.836 | 1.00 | 21.33 |
| ATOM | 1365 | CG2 | ILE | A | 706 | −2.466 | 2.331 | 46.335 | 1.00 | 23.53 |
| ATOM | 1366 | CG1 | ILE | A | 706 | −3.712 | 4.507 | 46.411 | 1.00 | 21.96 |
| ATOM | 1367 | CD1 | ILE | A | 706 | −3.892 | 4.577 | 47.918 | 1.00 | 22.24 |
| ATOM | 1368 | C | ILE | A | 706 | −3.415 | 1.782 | 43.685 | 1.00 | 16.48 |
| ATOM | 1369 | O | ILE | A | 706 | −2.291 | 1.470 | 43.291 | 1.00 | 18.62 |
| ATOM | 1370 | N | GLU | A | 707 | −4.456 | 0.962 | 43.584 | 1.00 | 15.92 |
| ATOM | 1371 | CA | GLU | A | 707 | −4.304 | −0.376 | 43.019 | 1.00 | 14.53 |
| ATOM | 1372 | CB | GLU | A | 707 | −5.509 | −1.250 | 43.378 | 1.00 | 19.33 |
| ATOM | 1373 | CG | GLU | A | 707 | −5.518 | −1.682 | 44.836 | 1.00 | 24.82 |
| ATOM | 1374 | CD | GLU | A | 707 | −4.269 | −2.460 | 45.214 | 1.00 | 31.26 |
| ATOM | 1375 | OE1 | GLU | A | 707 | −4.029 | −3.525 | 44.610 | 1.00 | 34.47 |
| ATOM | 1376 | OE2 | GLU | A | 707 | −3.525 | −2.006 | 46.110 | 1.00 | 30.79 |
| ATOM | 1377 | C | GLU | A | 707 | −4.093 | −0.385 | 41.507 | 1.00 | 14.27 |
| ATOM | 1378 | O | GLU | A | 707 | −3.283 | −1.156 | 41.002 | 1.00 | 15.02 |
| ATOM | 1379 | N | GLU | A | 708 | −4.818 | 0.458 | 40.782 | 1.00 | 11.81 |
| ATOM | 1380 | CA | GLU | A | 708 | −4.644 | 0.495 | 39.337 | 1.00 | 12.49 |
| ATOM | 1381 | CB | GLU | A | 708 | −5.756 | 1.317 | 38.673 | 1.00 | 14.73 |
| ATOM | 1382 | CG | GLU | A | 708 | −7.134 | 0.663 | 38.813 | 1.00 | 11.98 |
| ATOM | 1383 | CD | GLU | A | 708 | −8.088 | 1.020 | 37.683 | 1.00 | 18.01 |
| ATOM | 1384 | OE1 | GLU | A | 708 | −8.121 | 2.196 | 37.274 | 1.00 | 19.76 |
| ATOM | 1385 | OE2 | GLU | A | 708 | −8.816 | 0.120 | 37.216 | 1.00 | 22.22 |
| ATOM | 1386 | C | GLU | A | 708 | −3.264 | 1.054 | 38.996 | 1.00 | 14.00 |
| ATOM | 1387 | O | GLU | A | 708 | −2.624 | 0.607 | 38.047 | 1.00 | 12.79 |
| ATOM | 1388 | N | TYR | A | 709 | −2.798 | 2.018 | 39.784 | 1.00 | 12.70 |
| ATOM | 1389 | CA | TYR | A | 709 | −1.482 | 2.604 | 39.551 | 1.00 | 13.42 |
| ATOM | 1390 | CB | TYR | A | 709 | −1.204 | 3.724 | 40.557 | 1.00 | 12.05 |
| ATOM | 1391 | CG | TYR | A | 709 | 0.129 | 4.407 | 40.339 | 1.00 | 19.13 |
| ATOM | 1392 | CD1 | TYR | A | 709 | 0.311 | 5.302 | 39.296 | 1.00 | 14.81 |
| ATOM | 1393 | CE1 | TYR | A | 709 | 1.530 | 5.928 | 39.091 | 1.00 | 15.81 |
| ATOM | 1394 | CD2 | TYR | A | 709 | 1.207 | 4.149 | 41.174 | 1.00 | 17.39 |
| ATOM | 1395 | CE2 | TYR | A | 709 | 2.427 | 4.767 | 40.978 | 1.00 | 18.97 |
| ATOM | 1396 | CZ | TYR | A | 709 | 2.583 | 5.658 | 39.934 | 1.00 | 17.67 |
| ATOM | 1397 | OH | TYR | A | 709 | 3.793 | 6.284 | 39.737 | 1.00 | 18.77 |
| ATOM | 1398 | C | TYR | A | 709 | −0.424 | 1.515 | 39.708 | 1.00 | 15.44 |
| ATOM | 1399 | O | TYR | A | 709 | 0.452 | 1.348 | 38.855 | 1.00 | 15.06 |
| ATOM | 1400 | N | LYS | A | 710 | −0.520 | 0.774 | 40.808 | 1.00 | 15.25 |
| ATOM | 1401 | CA | LYS | A | 710 | 0.409 | −0.311 | 41.095 | 1.00 | 15.31 |
| ATOM | 1402 | CB | LYS | A | 710 | −0.020 | −1.026 | 42.379 | 1.00 | 22.10 |
| ATOM | 1403 | CG | LYS | A | 710 | 0.809 | −2.249 | 42.716 | 1.00 | 23.74 |
| ATOM | 1404 | CD | LYS | A | 710 | 0.299 | −2.931 | 43.978 | 1.00 | 33.24 |
| ATOM | 1405 | CE | LYS | A | 710 | 0.996 | −4.263 | 44.204 | 1.00 | 39.08 |
| ATOM | 1406 | NZ | LYS | A | 710 | 2.478 | −4.117 | 44.248 | 1.00 | 42.37 |
| ATOM | 1407 | C | LYS | A | 710 | 0.474 | −1.309 | 39.939 | 1.00 | 14.94 |
| ATOM | 1408 | O | LYS | A | 710 | 1.555 | −1.645 | 39.448 | 1.00 | 12.87 |
| ATOM | 1409 | N | THR | A | 711 | −0.692 | −1.777 | 39.506 | 1.00 | 13.50 |
| ATOM | 1410 | CA | THR | A | 711 | −0.783 | −2.736 | 38.411 | 1.00 | 12.45 |
| ATOM | 1411 | CB | THR | A | 711 | −2.249 | −3.092 | 38.128 | 1.00 | 17.44 |
| ATOM | 1412 | OG1 | THR | A | 711 | −2.791 | −3.792 | 39.255 | 1.00 | 21.96 |
| ATOM | 1413 | CG2 | THR | A | 711 | −2.365 | −3.953 | 36.872 | 1.00 | 16.75 |
| ATOM | 1414 | C | THR | A | 711 | −0.161 | −2.198 | 37.129 | 1.00 | 11.72 |
| ATOM | 1415 | O | THR | A | 711 | 0.597 | −2.896 | 36.442 | 1.00 | 13.19 |
| ATOM | 1416 | N | THR | A | 712 | −0.487 | −0.954 | 36.809 | 1.00 | 11.15 |
| ATOM | 1417 | CA | THR | A | 712 | 0.015 | −0.319 | 35.599 | 1.00 | 13.99 |
| ATOM | 1418 | CB | THR | A | 712 | −0.598 | 1.081 | 35.424 | 1.00 | 9.97 |
| ATOM | 1419 | OG1 | THR | A | 712 | −2.029 | 0.967 | 35.357 | 1.00 | 14.40 |
| ATOM | 1420 | CG2 | THR | A | 712 | −0.088 | 1.729 | 34.133 | 1.00 | 12.92 |
| ATOM | 1421 | C | THR | A | 712 | 1.541 | −0.221 | 35.556 | 1.00 | 12.77 |
| ATOM | 1422 | O | THR | A | 712 | 2.158 | −0.596 | 34.559 | 1.00 | 12.54 |
| ATOM | 1423 | N | LEU | A | 713 | 2.156 | 0.275 | 36.625 | 1.00 | 12.50 |
| ATOM | 1424 | CA | LEU | A | 713 | 3.612 | 0.384 | 36.638 | 1.00 | 11.02 |
| ATOM | 1425 | CB | LEU | A | 713 | 4.105 | 1.023 | 37.940 | 1.00 | 13.58 |
| ATOM | 1426 | CG | LEU | A | 713 | 3.889 | 2.528 | 38.100 | 1.00 | 22.03 |
| ATOM | 1427 | CD1 | LEU | A | 713 | 4.754 | 3.042 | 39.250 | 1.00 | 21.64 |
| ATOM | 1428 | CD2 | LEU | A | 713 | 4.261 | 3.241 | 36.809 | 1.00 | 27.88 |
| ATOM | 1429 | C | LEU | A | 713 | 4.298 | −0.967 | 36.438 | 1.00 | 13.04 |
| ATOM | 1430 | O | LEU | A | 713 | 5.340 | −1.048 | 35.785 | 1.00 | 12.26 |
| ATOM | 1431 | N | LYS | A | 714 | 3.718 | −2.026 | 36.995 | 1.00 | 11.66 |
| ATOM | 1432 | CA | LYS | A | 714 | 4.304 | −3.356 | 36.852 | 1.00 | 12.24 |
| ATOM | 1433 | CB | LYS | A | 714 | 3.540 | −4.383 | 37.690 | 1.00 | 17.46 |
| ATOM | 1434 | CG | LYS | A | 714 | 3.582 | −4.123 | 39.182 | 1.00 | 27.51 |
| ATOM | 1435 | CD | LYS | A | 714 | 2.878 | −5.236 | 39.939 | 1.00 | 33.99 |
| ATOM | 1436 | CE | LYS | A | 714 | 2.899 | −4.988 | 41.433 | 1.00 | 42.93 |
| ATOM | 1437 | NZ | LYS | A | 714 | 2.222 | −6.092 | 42.168 | 1.00 | 46.33 |
| ATOM | 1438 | C | LYS | A | 714 | 4.284 | −3.784 | 35.390 | 1.00 | 12.70 |
| ATOM | 1439 | O | LYS | A | 714 | 5.259 | −4.338 | 34.886 | 1.00 | 12.91 |
| ATOM | 1440 | N | ILE | A | 715 | 3.168 | −3.525 | 34.717 | 1.00 | 10.95 |
| ATOM | 1441 | CA | ILE | A | 715 | 3.023 | −3.869 | 33.308 | 1.00 | 10.57 |
| ATOM | 1442 | CB | ILE | A | 715 | 1.586 | −3.590 | 32.820 | 1.00 | 11.39 |
| ATOM | 1443 | CG2 | ILE | A | 715 | 1.468 | −3.896 | 31.339 | 1.00 | 11.35 |
| ATOM | 1444 | CG1 | ILE | A | 715 | 0.596 | −4.450 | 33.613 | 1.00 | 12.69 |
| ATOM | 1445 | CD1 | ILE | A | 715 | −0.854 | −4.130 | 33.332 | 1.00 | 13.44 |
| ATOM | 1446 | C | ILE | A | 715 | 4.017 | −3.052 | 32.482 | 1.00 | 10.34 |
| ATOM | 1447 | O | ILE | A | 715 | 4.676 | −3.581 | 31.588 | 1.00 | 11.86 |
| ATOM | 1448 | N | ILE | A | 716 | 4.133 | −1.762 | 32.793 | 1.00 | 10.78 |
| ATOM | 1449 | CA | ILE | A | 716 | 5.069 | −0.886 | 32.081 | 1.00 | 9.90 |
| ATOM | 1450 | CB | ILE | A | 716 | 4.989 | 0.563 | 32.621 | 1.00 | 10.08 |
| ATOM | 1451 | CG2 | ILE | A | 716 | 6.171 | 1.387 | 32.115 | 1.00 | 10.86 |
| ATOM | 1452 | CG1 | ILE | A | 716 | 3.661 | 1.197 | 32.189 | 1.00 | 10.91 |
| ATOM | 1453 | CD1 | ILE | A | 716 | 3.413 | 2.564 | 32.792 | 1.00 | 11.27 |
| ATOM | 1454 | C | ILE | A | 716 | 6.508 | −1.393 | 32.196 | 1.00 | 8.77 |
| ATOM | 1455 | O | ILE | A | 716 | 7.229 | −1.487 | 31.199 | 1.00 | 9.34 |
| ATOM | 1456 | N | LYS | A | 717 | 6.926 | −1.724 | 33.413 | 1.00 | 9.18 |
| ATOM | 1457 | CA | LYS | A | 717 | 8.275 | −2.235 | 33.623 | 1.00 | 10.88 |
| ATOM | 1458 | CB | LYS | A | 717 | 8.489 | −2.606 | 35.087 | 1.00 | 12.47 |
| ATOM | 1459 | CG | LYS | A | 717 | 9.790 | −3.366 | 35.337 | 1.00 | 14.78 |
| ATOM | 1460 | CD | LYS | A | 717 | 9.865 | −3.864 | 36.775 | 1.00 | 17.70 |
| ATOM | 1461 | CE | LYS | A | 717 | 11.045 | −4.809 | 36.977 | 1.00 | 29.85 |
| ATOM | 1462 | NZ | LYS | A | 717 | 11.082 | −5.374 | 38.355 | 1.00 | 25.71 |
| ATOM | 1463 | C | LYS | A | 717 | 8.540 | −3.469 | 32.752 | 1.00 | 10.54 |
| ATOM | 1464 | O | LYS | A | 717 | 9.536 | −3.524 | 32.041 | 1.00 | 10.05 |
| ATOM | 1465 | N | GLN | A | 718 | 7.647 | −4.451 | 32.847 | 1.00 | 11.40 |
| ATOM | 1466 | CA | GLN | A | 718 | 7.774 | −5.682 | 32.067 | 1.00 | 11.80 |
| ATOM | 1467 | CB | GLN | A | 718 | 6.635 | −6.628 | 32.434 | 1.00 | 11.33 |
| ATOM | 1468 | CG | GLN | A | 718 | 6.683 | −6.965 | 33.899 | 1.00 | 14.75 |
| ATOM | 1469 | CD | GLN | A | 718 | 7.954 | −7.691 | 34.293 | 1.00 | 35.80 |
| ATOM | 1470 | OE1 | GLN | A | 718 | 8.774 | −7.176 | 35.053 | 1.00 | 49.66 |
| ATOM | 1471 | NE2 | GLN | A | 718 | 8.115 | −8.909 | 33.784 | 1.00 | 43.62 |
| ATOM | 1472 | C | GLN | A | 718 | 7.778 | −5.422 | 30.559 | 1.00 | 9.82 |
| ATOM | 1473 | O | GLN | A | 718 | 8.559 | −6.027 | 29.831 | 1.00 | 9.86 |
| ATOM | 1474 | N | ALA | A | 719 | 6.920 | −4.518 | 30.108 | 1.00 | 8.83 |
| ATOM | 1475 | CA | ALA | A | 719 | 6.842 | −4.205 | 28.686 | 1.00 | 10.49 |
| ATOM | 1476 | CB | ALA | A | 719 | 5.657 | −3.292 | 28.436 | 1.00 | 5.96 |
| ATOM | 1477 | C | ALA | A | 719 | 8.129 | −3.560 | 28.186 | 1.00 | 9.98 |
| ATOM | 1478 | O | ALA | A | 719 | 8.618 | −3.892 | 27.101 | 1.00 | 9.19 |
| ATOM | 1479 | N | ILE | A | 720 | 8.688 | −2.641 | 28.971 | 1.00 | 7.25 |
| ATOM | 1480 | CA | ILE | A | 720 | 9.920 | −1.988 | 28.548 | 1.00 | 8.66 |
| ATOM | 1481 | CB | ILE | A | 720 | 10.233 | −0.753 | 29.426 | 1.00 | 10.86 |
| ATOM | 1482 | CG2 | ILE | A | 720 | 11.616 | −0.193 | 29.086 | 1.00 | 11.64 |
| ATOM | 1483 | CG1 | ILE | A | 720 | 9.164 | 0.320 | 29.181 | 1.00 | 11.59 |
| ATOM | 1484 | CD1 | ILE | A | 720 | 9.367 | 1.605 | 29.976 | 1.00 | 12.21 |
| ATOM | 1485 | C | ILE | A | 720 | 11.085 | −2.974 | 28.580 | 1.00 | 10.02 |
| ATOM | 1486 | O | ILE | A | 720 | 11.883 | −3.040 | 27.641 | 1.00 | 8.95 |
| ATOM | 1487 | N | LEU | A | 721 | 11.178 | −3.762 | 29.643 | 1.00 | 8.10 |
| ATOM | 1488 | CA | LEU | A | 721 | 12.264 | −4.733 | 29.719 | 1.00 | 8.45 |
| ATOM | 1489 | CB | LEU | A | 721 | 12.251 | −5.446 | 31.077 | 1.00 | 10.55 |
| ATOM | 1490 | CG | LEU | A | 721 | 12.613 | −4.553 | 32.267 | 1.00 | 13.26 |
| ATOM | 1491 | CD1 | LEU | A | 721 | 12.539 | −5.350 | 33.574 | 1.00 | 15.78 |
| ATOM | 1492 | CD2 | LEU | A | 721 | 14.006 | −3.991 | 32.059 | 1.00 | 24.08 |
| ATOM | 1493 | C | LEU | A | 721 | 12.173 | −5.750 | 28.576 | 1.00 | 9.96 |
| ATOM | 1494 | O | LEU | A | 721 | 13.195 | −6.196 | 28.049 | 1.00 | 8.91 |
| ATOM | 1495 | N | ALA | A | 722 | 10.948 | −6.082 | 28.175 | 1.00 | 8.77 |
| ATOM | 1496 | CA | ALA | A | 722 | 10.715 | −7.047 | 27.100 | 1.00 | 8.74 |
| ATOM | 1497 | CB | ALA | A | 722 | 9.219 | −7.218 | 26.879 | 1.00 | 9.06 |
| ATOM | 1498 | C | ALA | A | 722 | 11.383 | −6.648 | 25.786 | 1.00 | 10.10 |
| ATOM | 1499 | O | ALA | A | 722 | 11.704 | −7.507 | 24.965 | 1.00 | 13.26 |
| ATOM | 1500 | N | THR | A | 723 | 11.598 | −5.350 | 25.585 | 1.00 | 8.97 |
| ATOM | 1501 | CA | THR | A | 723 | 12.213 | −4.884 | 24.344 | 1.00 | 10.94 |
| ATOM | 1502 | CB | THR | A | 723 | 11.943 | −3.374 | 24.127 | 1.00 | 8.53 |
| ATOM | 1503 | OG1 | THR | A | 723 | 12.542 | −2.615 | 25.181 | 1.00 | 8.41 |
| ATOM | 1504 | CG2 | THR | A | 723 | 10.437 | −3.114 | 24.118 | 1.00 | 8.22 |
| ATOM | 1505 | C | THR | A | 723 | 13.713 | −5.187 | 24.203 | 1.00 | 9.21 |
| ATOM | 1506 | O | THR | A | 723 | 14.327 | −4.862 | 23.186 | 1.00 | 12.03 |
| ATOM | 1507 | N | ASP | A | 724 | 14.308 | −5.797 | 25.223 | 1.00 | 9.75 |
| ATOM | 1508 | CA | ASP | A | 724 | 15.710 | −6.203 | 25.133 | 1.00 | 7.82 |
| ATOM | 1509 | CB | ASP | A | 724 | 16.257 | −6.533 | 26.525 | 1.00 | 12.18 |
| ATOM | 1510 | CG | ASP | A | 724 | 17.648 | −7.147 | 26.490 | 1.00 | 8.16 |
| ATOM | 1511 | OD1 | ASP | A | 724 | 18.232 | −7.299 | 25.394 | 1.00 | 11.70 |
| ATOM | 1512 | OD2 | ASP | A | 724 | 18.161 | −7.481 | 27.581 | 1.00 | 11.61 |
| ATOM | 1513 | C | ASP | A | 724 | 15.600 | −7.475 | 24.293 | 1.00 | 8.26 |
| ATOM | 1514 | O | ASP | A | 724 | 15.022 | −8.459 | 24.742 | 1.00 | 10.28 |
| ATOM | 1515 | N | LEU | A | 725 | 16.127 | −7.452 | 23.073 | 1.00 | 9.50 |
| ATOM | 1516 | CA | LEU | A | 725 | 16.037 | −8.611 | 22.189 | 1.00 | 8.83 |
| ATOM | 1517 | CB | LEU | A | 725 | 16.791 | −8.343 | 20.883 | 1.00 | 11.33 |
| ATOM | 1518 | CG | LEU | A | 725 | 16.365 | −9.236 | 19.717 | 1.00 | 16.17 |
| ATOM | 1519 | CD1 | LEU | A | 725 | 14.910 | −8.953 | 19.367 | 1.00 | 12.02 |
| ATOM | 1520 | CD2 | LEU | A | 725 | 17.262 | −8.974 | 18.516 | 1.00 | 20.09 |
| ATOM | 1521 | C | LEU | A | 725 | 16.573 | −9.880 | 22.849 | 1.00 | 12.30 |
| ATOM | 1522 | O | LEU | A | 725 | 16.179 | −10.994 | 22.498 | 1.00 | 11.52 |
| ATOM | 1523 | N | ALA | A | 726 | 17.476 | −9.712 | 23.806 | 1.00 | 11.08 |
| ATOM | 1524 | CA | ALA | A | 726 | 18.028 | −10.855 | 24.517 | 1.00 | 12.63 |
| ATOM | 1525 | CB | ALA | A | 726 | 19.104 | −10.394 | 25.489 | 1.00 | 21.41 |
| ATOM | 1526 | C | ALA | A | 726 | 16.918 | −11.583 | 25.274 | 1.00 | 12.40 |
| ATOM | 1527 | O | ALA | A | 726 | 16.914 | −12.813 | 25.354 | 1.00 | 13.56 |
| ATOM | 1528 | N | LEU | A | 727 | 15.985 | −10.823 | 25.843 | 1.00 | 12.79 |
| ATOM | 1529 | CA | LEU | A | 727 | 14.884 | −11.421 | 26.591 | 1.00 | 14.99 |
| ATOM | 1530 | CB | LEU | A | 727 | 14.206 | −10.380 | 27.485 | 1.00 | 11.71 |
| ATOM | 1531 | CG | LEU | A | 727 | 15.096 | −9.856 | 28.612 | 1.00 | 16.19 |
| ATOM | 1532 | CD1 | LEU | A | 727 | 14.302 | −8.925 | 29.511 | 1.00 | 13.74 |
| ATOM | 1533 | CD2 | LEU | A | 727 | 15.631 | −11.036 | 29.409 | 1.00 | 19.43 |
| ATOM | 1534 | C | LEU | A | 727 | 13.868 | −12.045 | 25.646 | 1.00 | 15.02 |
| ATOM | 1535 | O | LEU | A | 727 | 13.173 | −12.996 | 26.005 | 1.00 | 15.57 |
| ATOM | 1536 | N | TYR | A | 728 | 13.777 | −11.500 | 24.438 | 1.00 | 12.38 |
| ATOM | 1537 | CA | TYR | A | 728 | 12.862 | −12.038 | 23.435 | 1.00 | 12.71 |
| ATOM | 1538 | CB | TYR | A | 728 | 12.844 | −11.136 | 22.194 | 1.00 | 10.64 |
| ATOM | 1539 | CG | TYR | A | 728 | 12.303 | −11.823 | 20.960 | 1.00 | 12.57 |
| ATOM | 1540 | CD1 | TYR | A | 728 | 10.966 | −12.197 | 20.875 | 1.00 | 14.71 |
| ATOM | 1541 | CE1 | TYR | A | 728 | 10.473 | −12.854 | 19.752 | 1.00 | 12.42 |
| ATOM | 1542 | CD2 | TYR | A | 728 | 13.139 | −12.124 | 19.888 | 1.00 | 13.44 |
| ATOM | 1543 | CE2 | TYR | A | 728 | 12.659 | −12.777 | 18.763 | 1.00 | 13.13 |
| ATOM | 1544 | CZ | TYR | A | 728 | 11.329 | −13.142 | 18.699 | 1.00 | 15.27 |
| ATOM | 1545 | OH | TYR | A | 728 | 10.857 | −13.801 | 17.580 | 1.00 | 18.62 |
| ATOM | 1546 | C | TYR | A | 728 | 13.350 | −13.430 | 23.041 | 1.00 | 12.62 |
| ATOM | 1547 | O | TYR | A | 728 | 12.588 | −14.401 | 23.028 | 1.00 | 14.23 |
| ATOM | 1548 | N | ILE | A | 729 | 14.636 | −13.522 | 22.723 | 1.00 | 12.98 |
| ATOM | 1549 | CA | ILE | A | 729 | 15.232 | −14.788 | 22.327 | 1.00 | 13.19 |
| ATOM | 1550 | CB | ILE | A | 729 | 16.723 | −14.595 | 21.983 | 1.00 | 14.56 |
| ATOM | 1551 | CG2 | ILE | A | 729 | 17.395 | −15.942 | 21.797 | 1.00 | 19.39 |
| ATOM | 1552 | CG1 | ILE | A | 729 | 16.849 | −13.727 | 20.727 | 1.00 | 18.02 |
| ATOM | 1553 | CD1 | ILE | A | 729 | 18.268 | −13.309 | 20.406 | 1.00 | 26.38 |
| ATOM | 1554 | C | ILE | A | 729 | 15.088 | −15.826 | 23.439 | 1.00 | 15.70 |
| ATOM | 1555 | O | ILE | A | 729 | 14.830 | −17.001 | 23.178 | 1.00 | 14.36 |
| ATOM | 1556 | N | LYS | A | 730 | 15.227 | −15.375 | 24.681 | 1.00 | 15.21 |
| ATOM | 1557 | CA | LYS | A | 730 | 15.127 | −16.257 | 25.837 | 1.00 | 16.44 |
| ATOM | 1558 | CB | LYS | A | 730 | 15.657 | −15.528 | 27.075 | 1.00 | 21.43 |
| ATOM | 1559 | CG | LYS | A | 730 | 15.494 | −16.291 | 28.376 | 1.00 | 27.38 |
| ATOM | 1560 | CD | LYS | A | 730 | 16.133 | −15.528 | 29.523 | 1.00 | 31.22 |
| ATOM | 1561 | CE | LYS | A | 730 | 15.975 | −16.267 | 30.839 | 1.00 | 36.43 |
| ATOM | 1562 | NZ | LYS | A | 730 | 16.672 | −15.556 | 31.947 | 1.00 | 41.27 |
| ATOM | 1563 | C | LYS | A | 730 | 13.727 | −16.802 | 26.133 | 1.00 | 16.48 |
| ATOM | 1564 | O | LYS | A | 730 | 13.573 | −17.972 | 26.498 | 1.00 | 17.06 |
| ATOM | 1565 | N | ARG | A | 731 | 12.707 | −15.969 | 25.968 | 1.00 | 14.55 |
| ATOM | 1566 | CA | ARG | A | 731 | 11.347 | −16.395 | 26.283 | 1.00 | 15.57 |
| ATOM | 1567 | CB | ARG | A | 731 | 10.622 | −15.291 | 27.060 | 1.00 | 19.59 |
| ATOM | 1568 | CG | ARG | A | 731 | 11.309 | −14.842 | 28.335 | 1.00 | 18.25 |
| ATOM | 1569 | CD | ARG | A | 731 | 10.437 | −13.846 | 29.086 | 1.00 | 33.93 |
| ATOM | 1570 | NE | ARG | A | 731 | 9.209 | −14.462 | 29.588 | 1.00 | 32.78 |
| ATOM | 1571 | CZ | ARG | A | 731 | 8.168 | −13.781 | 30.058 | 1.00 | 29.98 |
| ATOM | 1572 | NH1 | ARG | A | 731 | 8.197 | −12.456 | 30.091 | 1.00 | 30.21 |
| ATOM | 1573 | NH2 | ARG | A | 731 | 7.097 | −14.426 | 30.501 | 1.00 | 36.09 |
| ATOM | 1574 | C | ARG | A | 731 | 10.461 | −16.807 | 25.118 | 1.00 | 12.75 |
| ATOM | 1575 | O | ARG | A | 731 | 9.436 | −17.454 | 25.325 | 1.00 | 15.30 |
| ATOM | 1576 | N | ARG | A | 732 | 10.841 | −16.445 | 23.897 | 1.00 | 13.21 |
| ATOM | 1577 | CA | ARG | A | 732 | 10.002 | −16.759 | 22.751 | 1.00 | 11.06 |
| ATOM | 1578 | CB | ARG | A | 732 | 10.589 | −16.162 | 21.474 | 1.00 | 10.97 |
| ATOM | 1579 | CG | ARG | A | 732 | 11.872 | −16.812 | 21.021 | 1.00 | 12.09 |
| ATOM | 1580 | CD | ARG | A | 732 | 12.325 | −16.192 | 19.724 | 1.00 | 12.79 |
| ATOM | 1581 | NE | ARG | A | 732 | 13.525 | −16.822 | 19.193 | 1.00 | 13.80 |
| ATOM | 1582 | CZ | ARG | A | 732 | 13.919 | −16.704 | 17.930 | 1.00 | 20.20 |
| ATOM | 1583 | NH1 | ARG | A | 732 | 13.201 | −15.980 | 17.077 | 1.00 | 23.11 |
| ATOM | 1584 | NE2 | ARG | A | 732 | 15.025 | −17.307 | 17.518 | 1.00 | 22.17 |
| ATOM | 1585 | C | ARG | A | 732 | 9.751 | −18.245 | 22.549 | 1.00 | 11.37 |
| ATOM | 1586 | O | ARG | A | 732 | 8.704 | −18.625 | 22.031 | 1.00 | 11.95 |
| ATOM | 1587 | N | GLY | A | 733 | 10.703 | −19.078 | 22.962 | 1.00 | 13.25 |
| ATOM | 1588 | CA | GLY | A | 733 | 10.543 | −20.512 | 22.804 | 1.00 | 13.40 |
| ATOM | 1589 | C | GLY | A | 733 | 9.298 | −21.009 | 23.508 | 1.00 | 14.10 |
| ATOM | 1590 | O | GLY | A | 733 | 8.543 | −21.823 | 22.970 | 1.00 | 15.18 |
| ATOM | 1591 | N | GLU | A | 734 | 9.080 | −20.512 | 24.719 | 1.00 | 11.89 |
| ATOM | 1592 | CA | GLU | A | 734 | 7.919 | −20.900 | 25.505 | 1.00 | 14.43 |
| ATOM | 1593 | CB | GLU | A | 734 | 7.988 | −20.264 | 26.895 | 1.00 | 14.05 |
| ATOM | 1594 | CG | GLU | A | 734 | 6.675 | −20.312 | 27.661 | 1.00 | 18.08 |
| ATOM | 1595 | CD | GLU | A | 734 | 6.810 | −19.828 | 29.091 | 1.00 | 21.97 |
| ATOM | 1596 | OE1 | GLU | A | 734 | 7.742 | −19.048 | 29.372 | 1.00 | 19.31 |
| ATOM | 1597 | OE2 | GLU | A | 734 | 5.977 | −20.221 | 29.932 | 1.00 | 32.96 |
| ATOM | 1598 | C | GLU | A | 734 | 6.632 | −20.478 | 24.806 | 1.00 | 14.65 |
| ATOM | 1599 | O | GLU | A | 734 | 5.663 | −21.235 | 24.760 | 1.00 | 14.67 |
| ATOM | 1600 | N | PHE | A | 735 | 6.631 | −19.264 | 24.267 | 1.00 | 15.01 |
| ATOM | 1601 | CA | PHE | A | 735 | 5.470 | −18.729 | 23.566 | 1.00 | 11.90 |
| ATOM | 1602 | CB | PHE | A | 735 | 5.743 | −17.278 | 23.157 | 1.00 | 11.17 |
| ATOM | 1603 | CG | PHE | A | 735 | 4.632 | −16.635 | 22.366 | 1.00 | 16.06 |
| ATOM | 1604 | CD1 | PHE | A | 735 | 3.354 | −16.515 | 22.892 | 1.00 | 16.32 |
| ATOM | 1605 | CD2 | PHE | A | 735 | 4.885 | −16.102 | 21.111 | 1.00 | 13.68 |
| ATOM | 1606 | CE1 | PHE | A | 735 | 2.355 | −15.869 | 22.178 | 1.00 | 13.80 |
| ATOM | 1607 | CE2 | PHE | A | 735 | 3.897 | −15.458 | 20.400 | 1.00 | 12.16 |
| ATOM | 1608 | CZ | PHE | A | 735 | 2.631 | −15.340 | 20.931 | 1.00 | 14.03 |
| ATOM | 1609 | C | PHE | A | 735 | 5.142 | −19.566 | 22.329 | 1.00 | 13.78 |
| ATOM | 1610 | O | PHE | A | 735 | 3.989 | −19.946 | 22.117 | 1.00 | 13.01 |
| ATOM | 1611 | N | PHE | A | 736 | 6.156 | −19.853 | 21.517 | 1.00 | 12.56 |
| ATOM | 1612 | CA | PHE | A | 736 | 5.952 | −20.632 | 20.299 | 1.00 | 14.09 |
| ATOM | 1613 | CB | PHE | A | 736 | 7.242 | −20.691 | 19.472 | 1.00 | 15.66 |
| ATOM | 1614 | CG | PHE | A | 736 | 7.752 | −19.342 | 19.031 | 1.00 | 17.77 |
| ATOM | 1615 | CD1 | PHE | A | 736 | 6.888 | −18.268 | 18.894 | 1.00 | 16.30 |
| ATOM | 1616 | CD2 | PHE | A | 736 | 9.092 | −19.166 | 18.701 | 1.00 | 14.18 |
| ATOM | 1617 | CE1 | PHE | A | 736 | 7.345 | −17.040 | 18.435 | 1.00 | 13.31 |
| ATOM | 1618 | CE2 | PHE | A | 736 | 9.558 | −17.939 | 18.242 | 1.00 | 13.12 |
| ATOM | 1619 | CZ | PHE | A | 736 | 8.686 | −16.877 | 18.106 | 1.00 | 17.60 |
| ATOM | 1620 | C | PHE | A | 736 | 5.483 | −22.054 | 20.598 | 1.00 | 14.02 |
| ATOM | 1621 | O | PHE | A | 736 | 4.653 | −22.608 | 19.874 | 1.00 | 15.27 |
| ATOM | 1622 | N | GLU | A | 737 | 6.022 | −22.640 | 21.661 | 1.00 | 15.53 |
| ATOM | 1623 | CA | GLU | A | 737 | 5.667 | −24.000 | 22.047 | 1.00 | 16.49 |
| ATOM | 1624 | CB | GLU | A | 737 | 6.598 | −24.493 | 23.155 | 1.00 | 18.16 |
| ATOM | 1625 | CG | GLU | A | 737 | 6.296 | −25.908 | 23.623 | 1.00 | 32.20 |
| ATOM | 1626 | CD | GLU | A | 737 | 7.266 | −26.397 | 24.685 | 1.00 | 44.41 |
| ATOM | 1627 | OE1 | GLU | A | 737 | 7.368 | −25.748 | 25.748 | 1.00 | 43.95 |
| ATOM | 1628 | OE2 | GLU | A | 737 | 7.926 | −27.433 | 24.457 | 1.00 | 45.00 |
| ATOM | 1629 | C | GLU | A | 737 | 4.219 | −24.105 | 22.505 | 1.00 | 16.86 |
| ATOM | 1630 | O | GLU | A | 737 | 3.517 | −25.050 | 22.137 | 1.00 | 18.64 |
| ATOM | 1631 | N | LEU | A | 738 | 3.774 | −23.144 | 23.308 | 1.00 | 15.93 |
| ATOM | 1632 | CA | LEU | A | 738 | 2.396 | −23.140 | 23.791 | 1.00 | 14.96 |
| ATOM | 1633 | CB | LEU | A | 738 | 2.137 | −21.910 | 24.665 | 1.00 | 13.51 |
| ATOM | 1634 | CG | LEU | A | 738 | 2.817 | −21.890 | 26.037 | 1.00 | 13.44 |
| ATOM | 1635 | CD1 | LEU | A | 738 | 2.813 | −20.474 | 26.589 | 1.00 | 14.08 |
| ATOM | 1636 | CD2 | LEU | A | 738 | 2.103 | −22.844 | 26.987 | 1.00 | 21.77 |
| ATOM | 1637 | C | LEU | A | 738 | 1.435 | −23.137 | 22.610 | 1.00 | 15.65 |
| ATOM | 1638 | O | LEU | A | 738 | 0.431 | −23.854 | 22.610 | 1.00 | 17.64 |
| ATOM | 1639 | N | ILE | A | 739 | 1.743 | −22.323 | 21.604 | 1.00 | 13.75 |
| ATOM | 1640 | CA | ILE | A | 739 | 0.904 | −22.236 | 20.415 | 1.00 | 14.28 |
| ATOM | 1641 | CB | ILE | A | 739 | 1.388 | −21.112 | 19.464 | 1.00 | 12.79 |
| ATOM | 1642 | CG2 | ILE | A | 739 | 0.686 | −21.224 | 18.118 | 1.00 | 23.84 |
| ATOM | 1643 | CG1 | ILE | A | 739 | 1.122 | −19.744 | 20.096 | 1.00 | 16.58 |
| ATOM | 1644 | CD1 | ILE | A | 739 | 1.656 | −18.576 | 19.279 | 1.00 | 13.81 |
| ATOM | 1645 | C | ILE | A | 739 | 0.925 | −23.557 | 19.654 | 1.00 | 16.17 |
| ATOM | 1646 | O | ILE | A | 739 | −0.126 | −24.107 | 19.309 | 1.00 | 18.24 |
| ATOM | 1647 | N | ARG | A | 740 | 2.124 | −24.075 | 19.410 | 1.00 | 15.35 |
| ATOM | 1648 | CA | ARG | A | 740 | 2.274 | −25.322 | 18.671 | 1.00 | 18.48 |
| ATOM | 1649 | CB | ARG | A | 740 | 3.758 | −25.648 | 18.474 | 1.00 | 23.15 |
| ATOM | 1650 | CG | ARG | A | 740 | 3.996 | −26.804 | 17.508 | 1.00 | 35.46 |
| ATOM | 1651 | CD | ARG | A | 740 | 5.475 | −27.059 | 17.224 | 1.00 | 39.66 |
| ATOM | 1652 | NE | ARG | A | 740 | 6.174 | −27.694 | 18.340 | 1.00 | 40.53 |
| ATOM | 1653 | CZ | ARG | A | 740 | 6.703 | −27.043 | 19.371 | 1.00 | 42.20 |
| ATOM | 1654 | NH1 | ARG | A | 740 | 6.623 | −25.721 | 19.441 | 1.00 | 52.88 |
| ATOM | 1655 | NH2 | ARG | A | 740 | 7.317 | −27.718 | 20.335 | 1.00 | 44.19 |
| ATOM | 1656 | C | ARG | A | 740 | 1.582 | −26.495 | 19.360 | 1.00 | 19.41 |
| ATOM | 1657 | O | ARG | A | 740 | 1.093 | −27.410 | 18.698 | 1.00 | 18.06 |
| ATOM | 1658 | N | LYS | A | 741 | 1.539 | −26.470 | 20.686 | 1.00 | 18.03 |
| ATOM | 1659 | CA | LYS | A | 741 | 0.906 | −27.553 | 21.430 | 1.00 | 18.11 |
| ATOM | 1660 | CB | LYS | A | 741 | 1.696 | −27.853 | 22.699 | 1.00 | 14.50 |
| ATOM | 1661 | CG | LYS | A | 741 | 3.074 | −28.426 | 22.451 | 1.00 | 21.67 |
| ATOM | 1662 | CD | LYS | A | 741 | 3.717 | −28.812 | 23.763 | 1.00 | 23.36 |
| ATOM | 1663 | CE | LYS | A | 741 | 5.087 | −29.430 | 23.558 | 1.00 | 27.38 |
| ATOM | 1664 | NZ | LYS | A | 741 | 5.715 | −29.747 | 24.871 | 1.00 | 23.91 |
| ATOM | 1665 | C | LYS | A | 741 | −0.540 | −27.248 | 21.798 | 1.00 | 17.80 |
| ATOM | 1666 | O | LYS | A | 741 | −1.134 | −27.940 | 22.623 | 1.00 | 18.63 |
| ATOM | 1667 | N | ASN | A | 742 | −1.102 | −26.214 | 21.183 | 1.00 | 17.40 |
| ATOM | 1668 | CA | ASN | A | 742 | −2.478 | −25.822 | 21.456 | 1.00 | 17.84 |
| ATOM | 1669 | CB | ASN | A | 742 | −3.443 | −26.841 | 20.849 | 1.00 | 16.88 |
| ATOM | 1670 | CG | ASN | A | 742 | −3.442 | −26.801 | 19.335 | 1.00 | 15.02 |
| ATOM | 1671 | OD1 | ASN | A | 742 | −3.741 | −25.772 | 18.733 | 1.00 | 28.97 |
| ATOM | 1672 | ND2 | ASN | A | 742 | −3.100 | −27.923 | 18.709 | 1.00 | 20.44 |
| ATOM | 1673 | C | ASN | A | 742 | −2.734 | −25.671 | 22.949 | 1.00 | 17.96 |
| ATOM | 1674 | O | ASN | A | 742 | −3.727 | −26.174 | 23.486 | 1.00 | 17.06 |
| ATOM | 1675 | N | GLN | A | 743 | −1.827 | −24.964 | 23.615 | 1.00 | 16.68 |
| ATOM | 1676 | CA | GLN | A | 743 | −1.937 | −24.727 | 25.045 | 1.00 | 17.92 |
| ATOM | 1677 | CB | GLN | A | 743 | −0.738 | −25.338 | 25.777 | 1.00 | 20.38 |
| ATOM | 1678 | CG | GLN | A | 743 | −0.461 | −26.787 | 25.408 | 1.00 | 28.60 |
| ATOM | 1679 | CD | GLN | A | 743 | 0.746 | −27.350 | 26.131 | 1.00 | 30.94 |
| ATOM | 1680 | OE1 | GLN | A | 743 | 1.775 | −26.682 | 26.255 | 1.00 | 32.52 |
| ATOM | 1681 | NE2 | GLN | A | 743 | 0.634 | −28.588 | 26.602 | 1.00 | 20.21 |
| ATOM | 1682 | C | GLN | A | 743 | −2.002 | −23.233 | 25.341 | 1.00 | 16.35 |
| ATOM | 1683 | O | GLN | A | 743 | −2.175 | −22.836 | 26.490 | 1.00 | 18.44 |
| ATOM | 1684 | N | PHE | A | 744 | −1.877 | −22.407 | 24.307 | 1.00 | 16.26 |
| ATOM | 1685 | CA | PHE | A | 744 | −1.916 | −20.959 | 24.501 | 1.00 | 19.79 |
| ATOM | 1686 | CB | PHE | A | 744 | −1.627 | −20.229 | 23.184 | 1.00 | 21.58 |
| ATOM | 1687 | CG | PHE | A | 744 | −1.465 | −18.740 | 23.340 | 1.00 | 17.97 |
| ATOM | 1688 | CD1 | PHE | A | 744 | −0.441 | −18.216 | 24.117 | 1.00 | 20.04 |
| ATOM | 1689 | CD2 | PHE | A | 744 | −2.345 | −17.865 | 22.720 | 1.00 | 24.29 |
| ATOM | 1690 | CE1 | PHE | A | 744 | −0.298 | −16.847 | 24.271 | 1.00 | 24.80 |
| ATOM | 1691 | CE2 | PHE | A | 744 | −2.208 | −16.499 | 22.869 | 1.00 | 20.38 |
| ATOM | 1692 | CZ | PHE | A | 744 | −1.184 | −15.988 | 23.647 | 1.00 | 26.24 |
| ATOM | 1693 | C | PHE | A | 744 | −3.265 | −20.515 | 25.060 | 1.00 | 19.74 |
| ATOM | 1694 | O | PHE | A | 744 | −4.316 | −20.860 | 24.526 | 1.00 | 21.03 |
| ATOM | 1695 | N | ASN | A | 745 | −3.221 | −19.746 | 26.142 | 1.00 | 19.11 |
| ATOM | 1696 | CA | ASN | A | 745 | −4.427 | −19.260 | 26.791 | 1.00 | 18.83 |
| ATOM | 1697 | CB | ASN | A | 745 | −4.893 | −20.270 | 27.839 | 1.00 | 14.11 |
| ATOM | 1698 | CG | ASN | A | 745 | −6.067 | −19.764 | 28.653 | 1.00 | 17.45 |
| ATOM | 1699 | OD1 | ASN | A | 745 | −6.924 | −19.042 | 28.143 | 1.00 | 22.19 |
| ATOM | 1700 | ND2 | ASN | A | 745 | −6.120 | −20.152 | 29.921 | 1.00 | 30.84 |
| ATOM | 1701 | C | ASN | A | 745 | −4.183 | −17.906 | 27.446 | 1.00 | 17.46 |
| ATOM | 1702 | O | ASN | A | 745 | −3.518 | −17.818 | 28.481 | 1.00 | 17.84 |
| ATOM | 1703 | N | LEU | A | 746 | −4.720 | −16.857 | 26.826 | 1.00 | 16.56 |
| ATOM | 1704 | CA | LEU | A | 746 | −4.573 | −15.497 | 27.323 | 1.00 | 18.48 |
| ATOM | 1705 | CB | LEU | A | 746 | −5.163 | −14.495 | 26.323 | 1.00 | 15.11 |
| ATOM | 1706 | CG | LEU | A | 746 | −4.319 | −14.192 | 25.082 | 1.00 | 17.98 |
| ATOM | 1707 | CD1 | LEU | A | 746 | −5.031 | −13.165 | 24.217 | 1.00 | 16.50 |
| ATOM | 1708 | CD2 | LEU | A | 746 | −2.960 | −13.664 | 25.507 | 1.00 | 17.71 |
| ATOM | 1709 | C | LEU | A | 746 | −5.212 | −15.284 | 28.688 | 1.00 | 21.08 |
| ATOM | 1710 | O | LEU | A | 746 | −4.911 | −14.304 | 29.366 | 1.00 | 20.60 |
| ATOM | 1711 | N | GLU | A | 747 | −6.098 | −16.192 | 29.089 | 1.00 | 21.14 |
| ATOM | 1712 | CA | GLU | A | 747 | −6.748 | −16.075 | 30.389 | 1.00 | 21.90 |
| ATOM | 1713 | CB | GLU | A | 747 | −7.916 | −17.058 | 30.504 | 1.00 | 20.33 |
| ATOM | 1714 | CG | GLU | A | 747 | −9.179 | −16.586 | 29.813 | 1.00 | 31.82 |
| ATOM | 1715 | CD | GLU | A | 747 | −10.324 | −17.569 | 29.959 | 1.00 | 40.12 |
| ATOM | 1716 | OE1 | GLU | A | 747 | −10.550 | −18.057 | 31.086 | 1.00 | 43.30 |
| ATOM | 1717 | OE2 | GLU | A | 747 | −11.003 | −17.846 | 28.949 | 1.00 | 40.94 |
| ATOM | 1718 | C | GLU | A | 747 | −5.740 | −16.331 | 31.498 | 1.00 | 22.98 |
| ATOM | 1719 | O | GLU | A | 747 | −5.885 | −15.826 | 32.612 | 1.00 | 23.46 |
| ATOM | 1720 | N | ASP | A | 748 | −4.719 | −17.124 | 31.188 | 1.00 | 21.44 |
| ATOM | 1721 | CA | ASP | A | 748 | −3.670 | −17.424 | 32.151 | 1.00 | 21.15 |
| ATOM | 1722 | CB | ASP | A | 748 | −2.800 | −18.579 | 31.651 | 1.00 | 19.25 |
| ATOM | 1723 | CG | ASP | A | 748 | −1.656 | −18.899 | 32.595 | 1.00 | 19.90 |
| ATOM | 1724 | OD1 | ASP | A | 748 | −0.710 | −18.088 | 32.692 | 1.00 | 26.86 |
| ATOM | 1725 | OD2 | ASP | A | 748 | −1.704 | −19.962 | 33.246 | 1.00 | 42.46 |
| ATOM | 1726 | C | ASP | A | 748 | −2.819 | −16.171 | 32.314 | 1.00 | 20.94 |
| ATOM | 1727 | O | ASP | A | 748 | −2.240 | −15.677 | 31.351 | 1.00 | 19.01 |
| ATOM | 1728 | N | PRO | A | 749 | −2.742 | −15.637 | 33.541 | 1.00 | 18.53 |
| ATOM | 1729 | CD | PRO | A | 749 | −3.443 | −16.108 | 34.750 | 1.00 | 26.08 |
| ATOM | 1730 | CA | PRO | A | 749 | −1.958 | −14.432 | 33.825 | 1.00 | 19.83 |
| ATOM | 1731 | CB | PRO | A | 749 | −2.009 | −14.346 | 35.347 | 1.00 | 25.90 |
| ATOM | 1732 | CG | PRO | A | 749 | −3.377 | −14.896 | 35.654 | 1.00 | 25.60 |
| ATOM | 1733 | C | PRO | A | 749 | −0.528 | −14.476 | 33.286 | 1.00 | 17.96 |
| ATOM | 1734 | O | PRO | A | 749 | −0.047 | −13.506 | 32.708 | 1.00 | 18.09 |
| ATOM | 1735 | N | HIS | A | 750 | 0.149 | −15.603 | 33.475 | 1.00 | 17.27 |
| ATOM | 1736 | CA | HIS | A | 750 | 1.520 | −15.737 | 32.998 | 1.00 | 16.70 |
| ATOM | 1737 | CB | HIS | A | 750 | 2.138 | −17.039 | 33.509 | 1.00 | 14.57 |
| ATOM | 1738 | CG | HIS | A | 750 | 3.506 | −17.301 | 32.975 | 1.00 | 13.17 |
| ATOM | 1739 | CD2 | HIS | A | 750 | 4.710 | −16.780 | 33.299 | 1.00 | 15.22 |
| ATOM | 1740 | ND1 | HIS | A | 750 | 3.748 | −18.181 | 31.933 | 1.00 | 20.55 |
| ATOM | 1741 | CE1 | HIS | A | 750 | 5.030 | −18.183 | 31.649 | 1.00 | 21.44 |
| ATOM | 1742 | NE2 | HIS | A | 750 | 5.644 | −17.338 | 32.465 | 1.00 | 27.33 |
| ATOM | 1743 | C | HIS | A | 750 | 1.615 | −15.691 | 31.475 | 1.00 | 16.04 |
| ATOM | 1744 | O | HIS | A | 750 | 2.521 | −15.069 | 30.925 | 1.00 | 16.50 |
| ATOM | 1745 | N | GLU | A | 751 | 0.680 | −16.346 | 30.794 | 1.00 | 14.76 |
| ATOM | 1746 | CA | GLU | A | 751 | 0.689 | −16.359 | 29.333 | 1.00 | 14.97 |
| ATOM | 1747 | CB | GLU | A | 751 | −0.269 | −17.427 | 28.798 | 1.00 | 11.89 |
| ATOM | 1748 | CG | GLU | A | 751 | 0.173 | −18.848 | 29.123 | 1.00 | 16.94 |
| ATOM | 1749 | CD | GLU | A | 751 | −0.601 | −19.892 | 28.354 | 1.00 | 17.06 |
| ATOM | 1750 | OE1 | GLU | A | 751 | −0.804 | −19.697 | 27.140 | 1.00 | 17.15 |
| ATOM | 1751 | OE2 | GLU | A | 751 | −0.993 | −20.912 | 28.959 | 1.00 | 27.83 |
| ATOM | 1752 | C | GLU | A | 751 | 0.325 | −14.988 | 28.764 | 1.00 | 14.11 |
| ATOM | 1753 | O | GLU | A | 751 | 0.782 | −14.616 | 27.685 | 1.00 | 14.18 |
| ATOM | 1754 | N | LYS | A | 752 | −0.501 | −14.248 | 29.498 | 1.00 | 13.06 |
| ATOM | 1755 | CA | LYS | A | 752 | −0.900 | −12.910 | 29.092 | 1.00 | 11.63 |
| ATOM | 1756 | CB | LYS | A | 752 | −1.984 | −12.370 | 30.041 | 1.00 | 11.74 |
| ATOM | 1757 | CG | LYS | A | 752 | −2.200 | −10.865 | 29.981 | 1.00 | 11.33 |
| ATOM | 1758 | CD | LYS | A | 752 | −2.667 | −10.402 | 28.608 | 1.00 | 18.51 |
| ATOM | 1759 | CE | LYS | A | 752 | −3.921 | −11.138 | 28.184 | 1.00 | 31.49 |
| ATOM | 1760 | NZ | LYS | A | 752 | −4.536 | −10.523 | 26.978 | 1.00 | 47.29 |
| ATOM | 1761 | C | LYS | A | 752 | 0.362 | −12.047 | 29.176 | 1.00 | 14.58 |
| ATOM | 1762 | O | LYS | A | 752 | 0.665 | −11.293 | 28.249 | 1.00 | 11.11 |
| ATOM | 1763 | N | GLU | A | 753 | 1.093 | −12.196 | 30.270 | 1.00 | 12.46 |
| ATOM | 1764 | CA | GLU | A | 753 | 2.315 | −11.409 | 30.445 | 1.00 | 13.89 |
| ATOM | 1765 | CB | GLU | A | 753 | 2.939 | −11.745 | 31.801 | 1.00 | 15.41 |
| ATOM | 1766 | CG | GLU | A | 753 | 4.187 | −10.950 | 32.098 | 1.00 | 17.51 |
| ATOM | 1767 | CD | GLU | A | 753 | 4.946 | −11.478 | 33.300 | 1.00 | 36.21 |
| ATOM | 1768 | OE1 | GLU | A | 753 | 4.335 | −11.579 | 34.384 | 1.00 | 28.23 |
| ATOM | 1769 | OE2 | GLU | A | 753 | 6.150 | −11.782 | 33.162 | 1.00 | 36.81 |
| ATOM | 1770 | C | GLU | A | 753 | 3.267 | −11.729 | 29.313 | 1.00 | 14.45 |
| ATOM | 1771 | O | GLU | A | 753 | 3.852 | −10.839 | 28.671 | 1.00 | 11.34 |
| ATOM | 1772 | N | LEU | A | 754 | 3.441 | −13.018 | 29.035 | 1.00 | 12.90 |
| ATOM | 1773 | CA | LEU | A | 754 | 4.329 | −13.471 | 27.974 | 1.00 | 13.51 |
| ATOM | 1774 | CB | LEU | A | 754 | 4.377 | −15.011 | 27.908 | 1.00 | 13.76 |
| ATOM | 1775 | CG | LEU | A | 754 | 5.196 | −15.682 | 26.793 | 1.00 | 15.32 |
| ATOM | 1776 | CD1 | LEU | A | 754 | 6.663 | −15.286 | 26.877 | 1.00 | 15.56 |
| ATOM | 1777 | CD2 | LEU | A | 754 | 5.044 | −17.193 | 26.930 | 1.00 | 14.90 |
| ATOM | 1778 | C | LEU | A | 754 | 3.915 | −12.900 | 26.616 | 1.00 | 12.21 |
| ATOM | 1779 | O | LEU | A | 754 | 4.765 | −12.478 | 25.821 | 1.00 | 11.37 |
| ATOM | 1780 | N | PHE | A | 755 | 2.613 | −12.867 | 26.351 | 1.00 | 11.00 |
| ATOM | 1781 | CA | PHE | A | 755 | 2.119 | −12.336 | 25.088 | 1.00 | 9.92 |
| ATOM | 1782 | CB | PHE | A | 755 | 0.609 | −12.551 | 24.966 | 1.00 | 12.44 |
| ATOM | 1783 | CG | PHE | A | 755 | 0.002 | −11.867 | 23.780 | 1.00 | 11.67 |
| ATOM | 1784 | CD1 | PHE | A | 755 | 0.338 | −12.259 | 22.492 | 1.00 | 13.36 |
| ATOM | 1785 | CD2 | PHE | A | 755 | −0.858 | −10.792 | 23.948 | 1.00 | 9.93 |
| ATOM | 1786 | CE1 | PHE | A | 755 | −0.170 | −11.590 | 21.393 | 1.00 | 14.56 |
| ATOM | 1787 | CE2 | PHE | A | 755 | −1.369 | −10.118 | 22.854 | 1.00 | 13.93 |
| ATOM | 1788 | CZ | PHE | A | 755 | −1.021 | −10.517 | 21.574 | 1.00 | 18.65 |
| ATOM | 1789 | C | PHE | A | 755 | 2.431 | −10.840 | 24.972 | 1.00 | 9.95 |
| ATOM | 1790 | O | PHE | A | 755 | 2.856 | −10.367 | 23.916 | 1.00 | 10.28 |
| ATOM | 1791 | N | LEU | A | 756 | 2.225 | −10.099 | 26.057 | 1.00 | 9.98 |
| ATOM | 1792 | CA | LEU | A | 756 | 2.489 | −8.664 | 26.034 | 1.00 | 9.03 |
| ATOM | 1793 | CB | LEU | A | 756 | 2.102 | −8.017 | 27.370 | 1.00 | 9.14 |
| ATOM | 1794 | CG | LEU | A | 756 | 0.610 | −8.119 | 27.706 | 1.00 | 13.27 |
| ATOM | 1795 | CD1 | LEU | A | 756 | 0.331 | −7.487 | 29.065 | 1.00 | 21.21 |
| ATOM | 1796 | CD2 | LEU | A | 756 | −0.198 | −7.432 | 26.613 | 1.00 | 18.57 |
| ATOM | 1797 | C | LEU | A | 756 | 3.959 | −8.410 | 25.721 | 1.00 | 9.36 |
| ATOM | 1798 | O | LEU | A | 756 | 4.291 | −7.437 | 25.045 | 1.00 | 8.42 |
| ATOM | 1799 | N | ALA | A | 757 | 4.844 | −9.285 | 26.197 | 1.00 | 7.41 |
| ATOM | 1800 | CA | ALA | A | 757 | 6.267 | −9.118 | 25.915 | 1.00 | 10.17 |
| ATOM | 1801 | CB | ALA | A | 757 | 7.096 | −10.112 | 26.720 | 1.00 | 10.57 |
| ATOM | 1802 | C | ALA | A | 757 | 6.514 | −9.315 | 24.421 | 1.00 | 9.99 |
| ATOM | 1803 | O | ALA | A | 757 | 7.254 | −8.548 | 23.798 | 1.00 | 10.22 |
| ATOM | 1804 | N | MET | A | 758 | 5.890 | −10.341 | 23.845 | 1.00 | 8.41 |
| ATOM | 1805 | CA | MET | A | 758 | 6.046 | −10.627 | 22.422 | 1.00 | 8.55 |
| ATOM | 1806 | CB | MET | A | 758 | 5.348 | −11.946 | 22.061 | 1.00 | 8.57 |
| ATOM | 1807 | CG | MET | A | 758 | 5.898 | −13.175 | 22.783 | 1.00 | 9.97 |
| ATOM | 1808 | SD | MET | A | 758 | 7.613 | −13.597 | 22.367 | 1.00 | 15.99 |
| ATOM | 1809 | CE | MET | A | 758 | 7.436 | −14.226 | 20.702 | 1.00 | 36.66 |
| ATOM | 1810 | C | MET | A | 758 | 5.469 | −9.499 | 21.572 | 1.00 | 9.31 |
| ATOM | 1811 | O | MET | A | 758 | 6.035 | −9.134 | 20.534 | 1.00 | 8.67 |
| ATOM | 1812 | N | LEU | A | 759 | 4.334 | −8.962 | 22.008 | 1.00 | 10.68 |
| ATOM | 1813 | CA | LEU | A | 759 | 3.682 | −7.877 | 21.290 | 1.00 | 7.26 |
| ATOM | 1814 | CB | LEU | A | 759 | 2.348 | −7.536 | 21.954 | 1.00 | 6.70 |
| ATOM | 1815 | CG | LEU | A | 759 | 1.521 | −6.419 | 21.316 | 1.00 | 9.88 |
| ATOM | 1816 | CD1 | LEU | A | 759 | 1.217 | −6.751 | 19.858 | 1.00 | 13.46 |
| ATOM | 1817 | CD2 | LEU | A | 759 | 0.232 | −6.241 | 22.103 | 1.00 | 8.91 |
| ATOM | 1818 | C | LEU | A | 759 | 4.589 | −6.650 | 21.269 | 1.00 | 10.65 |
| ATOM | 1819 | O | LEU | A | 759 | 4.677 | −5.945 | 20.259 | 1.00 | 8.66 |
| ATOM | 1820 | N | MET | A | 760 | 5.258 | −6.388 | 22.387 | 1.00 | 8.87 |
| ATOM | 1821 | CA | MET | A | 760 | 6.168 | −5.251 | 22.454 | 1.00 | 9.10 |
| ATOM | 1822 | CB | MET | A | 760 | 6.760 | −5.114 | 23.859 | 1.00 | 9.37 |
| ATOM | 1823 | CG | MET | A | 760 | 5.831 | −4.491 | 24.889 | 1.00 | 7.94 |
| ATOM | 1824 | SD | MET | A | 760 | 5.392 | −2.762 | 24.513 | 1.00 | 11.15 |
| ATOM | 1825 | CE | MET | A | 760 | 6.981 | −1.951 | 24.748 | 1.00 | 10.58 |
| ATOM | 1826 | C | MET | A | 760 | 7.292 | −5.445 | 21.444 | 1.00 | 9.36 |
| ATOM | 1827 | O | MET | A | 760 | 7.693 | −4.501 | 20.757 | 1.00 | 8.44 |
| ATOM | 1828 | N | THR | A | 761 | 7.812 | −6.667 | 21.359 | 1.00 | 8.62 |
| ATOM | 1829 | CA | THR | A | 761 | 8.890 | −6.948 | 20.416 | 1.00 | 9.46 |
| ATOM | 1830 | CB | THR | A | 761 | 9.442 | −8.379 | 20.597 | 1.00 | 12.42 |
| ATOM | 1831 | OG1 | THR | A | 761 | 9.916 | −8.540 | 21.940 | 1.00 | 11.86 |
| ATOM | 1832 | CG2 | THR | A | 761 | 10.598 | −8.636 | 19.620 | 1.00 | 9.90 |
| ATOM | 1833 | C | THR | A | 761 | 8.389 | −6.785 | 18.984 | 1.00 | 10.31 |
| ATOM | 1834 | O | THR | A | 761 | 9.088 | −6.238 | 18.128 | 1.00 | 10.55 |
| ATOM | 1835 | N | ALA | A | 762 | 7.172 | −7.254 | 18.722 | 1.00 | 10.42 |
| ATOM | 1836 | CA | ALA | A | 762 | 6.598 | −7.148 | 17.389 | 1.00 | 7.73 |
| ATOM | 1837 | CB | ALA | A | 762 | 5.202 | −7.777 | 17.362 | 1.00 | 10.79 |
| ATOM | 1838 | C | ALA | A | 762 | 6.524 | −5.689 | 16.948 | 1.00 | 9.57 |
| ATOM | 1839 | O | ALA | A | 762 | 6.794 | −5.370 | 15.787 | 1.00 | 9.59 |
| ATOM | 1840 | N | CYS | A | 763 | 6.161 | −4.801 | 17.872 | 1.00 | 7.53 |
| ATOM | 1841 | CA | CYS | A | 763 | 6.063 | −3.378 | 17.551 | 1.00 | 7.89 |
| ATOM | 1842 | CB | CYS | A | 763 | 5.237 | −2.641 | 18.611 | 1.00 | 11.70 |
| ATOM | 1843 | SG | CYS | A | 763 | 3.503 | −3.143 | 18.670 | 1.00 | 11.19 |
| ATOM | 1844 | C | CYS | A | 763 | 7.441 | −2.744 | 17.443 | 1.00 | 9.03 |
| ATOM | 1845 | O | CYS | A | 763 | 7.689 | −1.911 | 16.574 | 1.00 | 10.26 |
| ATOM | 1846 | N | ASP | A | 764 | 8.340 | −3.157 | 18.329 | 1.00 | 8.73 |
| ATOM | 1847 | CA | ASP | A | 764 | 9.703 | −2.641 | 18.352 | 1.00 | 10.03 |
| ATOM | 1848 | CB | ASP | A | 764 | 10.448 | −3.269 | 19.541 | 1.00 | 11.85 |
| ATOM | 1849 | CG | ASP | A | 764 | 11.766 | −2.585 | 19.840 | 1.00 | 12.03 |
| ATOM | 1850 | OD1 | ASP | A | 764 | 11.963 | −1.451 | 19.361 | 1.00 | 11.46 |
| ATOM | 1851 | OD2 | ASP | A | 764 | 12.592 | −3.176 | 20.571 | 1.00 | 15.03 |
| ATOM | 1852 | C | ASP | A | 764 | 10.447 | −2.924 | 17.036 | 1.00 | 10.20 |
| ATOM | 1853 | O | ASP | A | 764 | 11.225 | −2.097 | 16.562 | 1.00 | 11.71 |
| ATOM | 1854 | N | LEU | A | 765 | 10.194 | −4.086 | 16.442 | 1.00 | 11.54 |
| ATOM | 1855 | CA | LEU | A | 765 | 10.867 | −4.474 | 15.202 | 1.00 | 11.71 |
| ATOM | 1856 | CB | LEU | A | 765 | 11.208 | −5.965 | 15.252 | 1.00 | 13.42 |
| ATOM | 1857 | CG | LEU | A | 765 | 12.017 | −6.454 | 16.453 | 1.00 | 15.02 |
| ATOM | 1858 | CD1 | LEU | A | 765 | 12.233 | −7.957 | 16.348 | 1.00 | 13.91 |
| ATOM | 1859 | CD2 | LEU | A | 765 | 13.345 | −5.721 | 16.499 | 1.00 | 16.31 |
| ATOM | 1860 | C | LEU | A | 765 | 10.049 | −4.221 | 13.941 | 1.00 | 12.10 |
| ATOM | 1861 | O | LEU | A | 765 | 10.490 | −4.560 | 12.837 | 1.00 | 11.30 |
| ATOM | 1862 | N | SER | A | 766 | 8.881 | −3.606 | 14.101 | 1.00 | 12.76 |
| ATOM | 1863 | CA | SER | A | 766 | 7.965 | −3.383 | 12.983 | 1.00 | 10.86 |
| ATOM | 1864 | CB | SER | A | 766 | 6.672 | −2.733 | 13.488 | 1.00 | 8.49 |
| ATOM | 1865 | OG | SER | A | 766 | 6.902 | −1.438 | 13.991 | 1.00 | 14.83 |
| ATOM | 1866 | C | SER | A | 766 | 8.446 | −2.657 | 11.729 | 1.00 | 7.60 |
| ATOM | 1867 | O | SER | A | 766 | 7.782 | −2.735 | 10.700 | 1.00 | 9.56 |
| ATOM | 1868 | N | ALA | A | 767 | 9.575 | −1.956 | 11.787 | 1.00 | 8.02 |
| ATOM | 1869 | CA | ALA | A | 767 | 10.073 | −1.296 | 10.581 | 1.00 | 9.45 |
| ATOM | 1870 | CB | ALA | A | 767 | 11.364 | −0.522 | 10.881 | 1.00 | 11.41 |
| ATOM | 1871 | C | ALA | A | 767 | 10.340 | −2.377 | 9.521 | 1.00 | 10.04 |
| ATOM | 1872 | O | ALA | A | 767 | 10.253 | −2.124 | 8.319 | 1.00 | 10.04 |
| ATOM | 1873 | N | ILE | A | 768 | 10.655 | −3.586 | 9.976 | 1.00 | 9.49 |
| ATOM | 1874 | CA | ILE | A | 768 | 10.942 | −4.694 | 9.066 | 1.00 | 12.99 |
| ATOM | 1875 | CB | ILE | A | 768 | 11.555 | −5.894 | 9.840 | 1.00 | 9.64 |
| ATOM | 1876 | CG2 | ILE | A | 768 | 10.473 | −6.631 | 10.612 | 1.00 | 7.91 |
| ATOM | 1877 | CG1 | ILE | A | 768 | 12.270 | −6.836 | 8.868 | 1.00 | 12.83 |
| ATOM | 1878 | CD1 | ILE | A | 768 | 13.471 | −6.199 | 8.190 | 1.00 | 15.69 |
| ATOM | 1879 | C | ILE | A | 768 | 9.704 | −5.173 | 8.296 | 1.00 | 11.57 |
| ATOM | 1880 | O | ILE | A | 768 | 9.812 | −5.986 | 7.376 | 1.00 | 12.45 |
| ATOM | 1881 | N | THR | A | 769 | 8.530 | −4.665 | 8.662 | 1.00 | 12.70 |
| ATOM | 1882 | CA | THR | A | 769 | 7.286 | −5.062 | 7.999 | 1.00 | 10.88 |
| ATOM | 1883 | CB | THR | A | 769 | 6.167 | −5.326 | 9.025 | 1.00 | 13.25 |
| ATOM | 1884 | OG1 | THR | A | 769 | 5.760 | −4.083 | 9.617 | 1.00 | 12.15 |
| ATOM | 1885 | CG2 | THR | A | 769 | 6.654 | −6.271 | 10.124 | 1.00 | 16.86 |
| ATOM | 1886 | C | THR | A | 769 | 6.762 | −3.991 | 7.036 | 1.00 | 12.18 |
| ATOM | 1887 | O | THR | A | 769 | 5.795 | −4.223 | 6.308 | 1.00 | 13.18 |
| ATOM | 1888 | N | LYS | A | 770 | 7.402 | −2.824 | 7.043 | 1.00 | 12.64 |
| ATOM | 1889 | CA | LYS | A | 770 | 6.986 | −1.686 | 6.216 | 1.00 | 10.43 |
| ATOM | 1890 | CB | LYS | A | 770 | 7.684 | −0.413 | 6.714 | 1.00 | 12.22 |
| ATOM | 1891 | CG | LYS | A | 770 | 7.375 | −0.029 | 8.165 | 1.00 | 7.87 |
| ATOM | 1892 | CD | LYS | A | 770 | 5.948 | 0.457 | 8.330 | 1.00 | 13.33 |
| ATOM | 1893 | CE | LYS | A | 770 | 5.644 | 0.766 | 9.794 | 1.00 | 14.40 |
| ATOM | 1894 | NZ | LYS | A | 770 | 4.263 | 1.293 | 9.973 | 1.00 | 14.45 |
| ATOM | 1895 | C | LYS | A | 770 | 7.247 | −1.829 | 4.716 | 1.00 | 11.91 |
| ATOM | 1896 | O | LYS | A | 770 | 8.069 | −2.638 | 4.290 | 1.00 | 13.63 |
| ATOM | 1897 | N | PRO | A | 771 | 6.529 | −1.041 | 3.892 | 1.00 | 11.52 |
| ATOM | 1898 | CD | PRO | A | 771 | 5.408 | −0.150 | 4.240 | 1.00 | 14.60 |
| ATOM | 1899 | CA | PRO | A | 771 | 6.718 | −1.096 | 2.441 | 1.00 | 12.80 |
| ATOM | 1900 | CB | PRO | A | 771 | 5.914 | 0.099 | 1.950 | 1.00 | 15.88 |
| ATOM | 1901 | CG | PRO | A | 771 | 4.751 | 0.098 | 2.886 | 1.00 | 11.15 |
| ATOM | 1902 | C | PRO | A | 771 | 8.206 | −0.933 | 2.183 | 1.00 | 14.66 |
| ATOM | 1903 | O | PRO | A | 771 | 8.880 | −0.201 | 2.908 | 1.00 | 15.01 |
| ATOM | 1904 | N | TRP | A | 772 | 8.711 | −1.604 | 1.156 | 1.00 | 14.26 |
| ATOM | 1905 | CA | TRP | A | 772 | 10.133 | −1.555 | 0.827 | 1.00 | 15.34 |
| ATOM | 1906 | CB | TRP | A | 772 | 10.367 | −2.121 | −0.578 | 1.00 | 14.10 |
| ATOM | 1907 | CG | TRP | A | 772 | 11.809 | −2.101 | −1.009 | 1.00 | 17.47 |
| ATOM | 1908 | CD2 | TRP | A | 772 | 12.931 | −2.659 | −0.307 | 1.00 | 17.61 |
| ATOM | 1909 | CE2 | TRP | A | 772 | 14.079 | −2.404 | −1.086 | 1.00 | 19.63 |
| ATOM | 1910 | CE3 | TRP | A | 772 | 13.076 | −3.348 | 0.901 | 1.00 | 16.69 |
| ATOM | 1911 | CD1 | TRP | A | 772 | 12.313 | −1.547 | −2.152 | 1.00 | 16.69 |
| ATOM | 1912 | NE1 | TRP | A | 772 | 13.675 | −1.725 | −2.204 | 1.00 | 24.41 |
| ATOM | 1913 | CZ2 | TRP | A | 772 | 15.354 | −2.816 | −0.694 | 1.00 | 24.29 |
| ATOM | 1914 | CZ3 | TRP | A | 772 | 14.344 | −3.755 | 1.289 | 1.00 | 17.48 |
| ATOM | 1915 | CH2 | TRP | A | 772 | 15.466 | −3.488 | 0.493 | 1.00 | 21.25 |
| ATOM | 1916 | C | TRP | A | 772 | 10.820 | −0.190 | 0.952 | 1.00 | 17.78 |
| ATOM | 1917 | O | TRP | A | 772 | 11.834 | −0.070 | 1.639 | 1.00 | 15.81 |
| ATOM | 1918 | N | PRO | A | 773 | 10.286 | 0.853 | 0.291 | 1.00 | 19.35 |
| ATOM | 1919 | CD | PRO | A | 773 | 9.132 | 0.877 | −0.624 | 1.00 | 18.85 |
| ATOM | 1920 | CA | PRO | A | 773 | 10.909 | 2.180 | 0.377 | 1.00 | 18.24 |
| ATOM | 1921 | CB | PRO | A | 773 | 9.926 | 3.068 | −0.382 | 1.00 | 21.17 |
| ATOM | 1922 | CG | PRO | A | 773 | 9.386 | 2.138 | −1.422 | 1.00 | 23.60 |
| ATOM | 1923 | C | PRO | A | 773 | 11.129 | 2.654 | 1.814 | 1.00 | 16.33 |
| ATOM | 1924 | O | PRO | A | 773 | 12.167 | 3.235 | 2.140 | 1.00 | 16.01 |
| ATOM | 1925 | N | ILE | A | 774 | 10.143 | 2.406 | 2.668 | 1.00 | 16.23 |
| ATOM | 1926 | CA | ILE | A | 774 | 10.228 | 2.805 | 4.066 | 1.00 | 15.30 |
| ATOM | 1927 | CB | ILE | A | 774 | 8.851 | 2.670 | 4.751 | 1.00 | 15.99 |
| ATOM | 1928 | CG2 | ILE | A | 774 | 8.977 | 2.922 | 6.250 | 1.00 | 13.24 |
| ATOM | 1929 | CG1 | ILE | A | 774 | 7.867 | 3.658 | 4.110 | 1.00 | 20.68 |
| ATOM | 1930 | CD1 | ILE | A | 774 | 6.441 | 3.501 | 4.588 | 1.00 | 21.74 |
| ATOM | 1931 | C | ILE | A | 774 | 11.264 | 1.958 | 4.804 | 1.00 | 16.31 |
| ATOM | 1932 | O | ILE | A | 774 | 12.133 | 2.491 | 5.497 | 1.00 | 15.63 |
| ATOM | 1933 | N | GLN | A | 775 | 11.173 | 0.640 | 4.650 | 1.00 | 13.80 |
| ATOM | 1934 | CA | GLN | A | 775 | 12.120 | −0.274 | 5.279 | 1.00 | 12.85 |
| ATOM | 1935 | CB | GLN | A | 775 | 11.786 | −1.727 | 4.901 | 1.00 | 13.34 |
| ATOM | 1936 | CG | GLN | A | 775 | 12.987 | −2.688 | 4.921 | 1.00 | 14.20 |
| ATOM | 1937 | CD | GLN | A | 775 | 13.582 | −2.897 | 6.305 | 1.00 | 21.70 |
| ATOM | 1938 | OE1 | GLN | A | 775 | 14.663 | −3.472 | 6.441 | 1.00 | 13.81 |
| ATOM | 1939 | NE2 | GLN | A | 775 | 12.880 | −2.440 | 7.336 | 1.00 | 14.50 |
| ATOM | 1940 | C | GLN | A | 775 | 13.549 | 0.057 | 4.877 | 1.00 | 13.13 |
| ATOM | 1941 | O | GLN | A | 775 | 14.458 | 0.060 | 5.694 | 1.00 | 12.67 |
| ATOM | 1942 | N | GLN | A | 776 | 13.749 | 0.352 | 3.590 | 1.00 | 10.04 |
| ATOM | 1943 | CA | GLN | A | 776 | 15.068 | 0.674 | 3.090 | 1.00 | 12.84 |
| ATOM | 1944 | CB | GLN | A | 776 | 15.004 | 0.820 | 1.564 | 1.00 | 19.00 |
| ATOM | 1945 | CG | GLN | A | 776 | 16.326 | 0.943 | 0.890 | 1.00 | 25.33 |
| ATOM | 1946 | CD | GLN | A | 776 | 16.172 | 0.760 | −0.595 | 1.00 | 36.37 |
| ATOM | 1947 | OE1 | GLN | A | 776 | 15.258 | 1.315 | −1.209 | 1.00 | 39.80 |
| ATOM | 1948 | NE2 | GLN | A | 776 | 17.058 | −0.023 | −1.186 | 1.00 | 41.16 |
| ATOM | 1949 | C | GLN | A | 776 | 15.627 | 1.934 | 3.738 | 1.00 | 10.61 |
| ATOM | 1950 | O | GLN | A | 776 | 16.796 | 1.988 | 4.109 | 1.00 | 13.39 |
| ATOM | 1951 | N | ARG | A | 777 | 14.776 | 2.946 | 3.922 | 1.00 | 10.52 |
| ATOM | 1952 | CA | ARG | A | 777 | 15.199 | 4.197 | 4.534 | 1.00 | 11.43 |
| ATOM | 1953 | CB | ARG | A | 777 | 14.090 | 5.253 | 4.419 | 1.00 | 15.26 |
| ATOM | 1954 | CG | ARG | A | 777 | 14.487 | 6.607 | 4.948 | 1.00 | 20.06 |
| ATOM | 1955 | CD | ARG | A | 777 | 13.343 | 7.586 | 4.765 | 1.00 | 25.19 |
| ATOM | 1956 | NE | ARG | A | 777 | 12.206 | 7.273 | 5.627 | 1.00 | 34.37 |
| ATOM | 1957 | CZ | ARG | A | 777 | 12.232 | 7.359 | 6.954 | 1.00 | 35.62 |
| ATOM | 1958 | NH1 | ARG | A | 777 | 13.340 | 7.747 | 7.575 | 1.00 | 37.87 |
| ATOM | 1959 | NH2 | ARG | A | 777 | 11.149 | 7.068 | 7.661 | 1.00 | 29.66 |
| ATOM | 1960 | C | ARG | A | 777 | 15.560 | 3.979 | 5.997 | 1.00 | 10.83 |
| ATOM | 1961 | O | ARG | A | 777 | 16.601 | 4.447 | 6.472 | 1.00 | 13.15 |
| ATOM | 1962 | N | ILE | A | 778 | 14.715 | 3.238 | 6.705 | 1.00 | 12.38 |
| ATOM | 1963 | CA | ILE | A | 778 | 14.953 | 2.944 | 8.112 | 1.00 | 13.18 |
| ATOM | 1964 | CB | ILE | A | 778 | 13.753 | 2.209 | 8.744 | 1.00 | 15.50 |
| ATOM | 1965 | CG2 | ILE | A | 778 | 14.091 | 1.788 | 10.170 | 1.00 | 13.56 |
| ATOM | 1966 | CG1 | ILE | A | 778 | 12.516 | 3.108 | 8.721 | 1.00 | 22.29 |
| ATOM | 1967 | CD1 | ILE | A | 778 | 12.685 | 4.399 | 9.478 | 1.00 | 26.81 |
| ATOM | 1968 | C | ILE | A | 778 | 16.198 | 2.086 | 8.305 | 1.00 | 12.49 |
| ATOM | 1969 | O | ILE | A | 778 | 16.963 | 2.298 | 9.245 | 1.00 | 12.70 |
| ATOM | 1970 | N | ALA | A | 779 | 16.399 | 1.117 | 7.417 | 1.00 | 12.47 |
| ATOM | 1971 | CA | ALA | A | 779 | 17.563 | 0.242 | 7.510 | 1.00 | 13.19 |
| ATOM | 1972 | CB | ALA | A | 779 | 17.538 | −0.789 | 6.391 | 1.00 | 14.05 |
| ATOM | 1973 | C | ALA | A | 779 | 18.856 | 1.051 | 7.452 | 1.00 | 13.71 |
| ATOM | 1974 | O | ALA | A | 779 | 19.839 | 0.700 | 8.106 | 1.00 | 11.99 |
| ATOM | 1975 | N | GLU | A | 780 | 18.858 | 2.131 | 6.672 | 1.00 | 14.86 |
| ATOM | 1976 | CA | GLU | A | 780 | 20.048 | 2.976 | 6.560 | 1.00 | 13.62 |
| ATOM | 1977 | CB | GLU | A | 780 | 19.860 | 4.053 | 5.487 | 1.00 | 17.29 |
| ATOM | 1978 | CG | GLU | A | 780 | 19.742 | 3.519 | 4.075 | 1.00 | 30.45 |
| ATOM | 1979 | CD | GLU | A | 780 | 19.744 | 4.628 | 3.038 | 1.00 | 42.90 |
| ATOM | 1980 | OE1 | GLU | A | 780 | 20.761 | 5.347 | 2.939 | 1.00 | 42.08 |
| ATOM | 1981 | OE2 | GLU | A | 780 | 18.728 | 4.783 | 2.326 | 1.00 | 56.88 |
| ATOM | 1982 | C | GLU | A | 780 | 20.350 | 3.649 | 7.897 | 1.00 | 13.45 |
| ATOM | 1983 | O | GLU | A | 780 | 21.506 | 3.791 | 8.277 | 1.00 | 14.75 |
| ATOM | 1984 | N | LEU | A | 781 | 19.303 | 4.074 | 8.597 | 1.00 | 12.82 |
| ATOM | 1985 | CA | LEU | A | 781 | 19.464 | 4.716 | 9.898 | 1.00 | 10.66 |
| ATOM | 1986 | CB | LEU | A | 781 | 18.113 | 5.215 | 10.410 | 1.00 | 12.60 |
| ATOM | 1987 | CG | LEU | A | 781 | 17.428 | 6.296 | 9.574 | 1.00 | 15.49 |
| ATOM | 1988 | CD1 | LEU | A | 781 | 16.101 | 6.670 | 10.215 | 1.00 | 14.52 |
| ATOM | 1989 | CD2 | LEU | A | 781 | 18.339 | 7.513 | 9.475 | 1.00 | 17.00 |
| ATOM | 1990 | C | LEU | A | 781 | 20.032 | 3.704 | 10.887 | 1.00 | 12.39 |
| ATOM | 1991 | O | LEU | A | 781 | 20.935 | 4.014 | 11.673 | 1.00 | 11.98 |
| ATOM | 1992 | N | VAL | A | 782 | 19.490 | 2.490 | 10.839 | 1.00 | 9.90 |
| ATOM | 1993 | CA | VAL | A | 782 | 19.933 | 1.418 | 11.719 | 1.00 | 9.65 |
| ATOM | 1994 | CB | VAL | A | 782 | 19.116 | 0.126 | 11.463 | 1.00 | 10.42 |
| ATOM | 1995 | CG1 | VAL | A | 782 | 19.714 | −1.029 | 12.240 | 1.00 | 13.17 |
| ATOM | 1996 | CG2 | VAL | A | 782 | 17.657 | 0.339 | 11.868 | 1.00 | 16.63 |
| ATOM | 1997 | C | VAL | A | 782 | 21.418 | 1.154 | 11.477 | 1.00 | 10.70 |
| ATOM | 1998 | O | VAL | A | 782 | 22.210 | 1.068 | 12.418 | 1.00 | 13.37 |
| ATOM | 1999 | N | ALA | A | 783 | 21.793 | 1.053 | 10.206 | 1.00 | 11.25 |
| ATOM | 2000 | CA | ALA | A | 783 | 23.185 | 0.810 | 9.839 | 1.00 | 11.83 |
| ATOM | 2001 | CB | ALA | A | 783 | 23.299 | 0.632 | 8.337 | 1.00 | 15.10 |
| ATOM | 2002 | C | ALA | A | 783 | 24.074 | 1.961 | 10.298 | 1.00 | 12.03 |
| ATOM | 2003 | O | ALA | A | 783 | 25.170 | 1.746 | 10.820 | 1.00 | 12.81 |
| ATOM | 2004 | N | THR | A | 784 | 23.599 | 3.187 | 10.099 | 1.00 | 11.94 |
| ATOM | 2005 | CA | THR | A | 784 | 24.358 | 4.359 | 10.504 | 1.00 | 10.90 |
| ATOM | 2006 | CB | THR | A | 784 | 23.572 | 5.659 | 10.211 | 1.00 | 12.78 |
| ATOM | 2007 | OG1 | THR | A | 784 | 23.359 | 5.778 | 8.797 | 1.00 | 15.62 |
| ATOM | 2008 | CG2 | THR | A | 784 | 24.341 | 6.883 | 10.706 | 1.00 | 13.70 |
| ATOM | 2009 | C | THR | A | 784 | 24.676 | 4.282 | 11.995 | 1.00 | 11.74 |
| ATOM | 2010 | O | THR | A | 784 | 25.819 | 4.490 | 12.408 | 1.00 | 10.51 |
| ATOM | 2011 | N | GLU | A | 785 | 23.670 | 3.969 | 12.805 | 1.00 | 10.01 |
| ATOM | 2012 | CA | GLU | A | 785 | 23.881 | 3.880 | 14.243 | 1.00 | 10.75 |
| ATOM | 2013 | CB | GLU | A | 785 | 22.549 | 3.669 | 14.968 | 1.00 | 7.51 |
| ATOM | 2014 | CG | GLU | A | 785 | 22.675 | 3.740 | 16.483 | 1.00 | 9.65 |
| ATOM | 2015 | CD | GLU | A | 785 | 21.335 | 3.755 | 17.178 | 1.00 | 7.52 |
| ATOM | 2016 | OE1 | GLU | A | 785 | 20.415 | 4.436 | 16.674 | 1.00 | 10.93 |
| ATOM | 2017 | OE2 | GLU | A | 785 | 21.208 | 3.099 | 18.236 | 1.00 | 12.56 |
| ATOM | 2018 | C | GLU | A | 785 | 24.847 | 2.760 | 14.612 | 1.00 | 11.99 |
| ATOM | 2019 | O | GLU | A | 785 | 25.737 | 2.948 | 15.440 | 1.00 | 11.68 |
| ATOM | 2020 | N | PHE | A | 786 | 24.671 | 1.594 | 13.997 | 1.00 | 9.83 |
| ATOM | 2021 | CA | PHE | A | 786 | 25.541 | 0.464 | 14.285 | 1.00 | 10.39 |
| ATOM | 2022 | CB | PHE | A | 786 | 25.069 | −0.794 | 13.550 | 1.00 | 11.58 |
| ATOM | 2023 | CG | PHE | A | 786 | 23.876 | −1.455 | 14.176 | 1.00 | 16.59 |
| ATOM | 2024 | CD1 | PHE | A | 786 | 23.632 | −1.327 | 15.533 | 1.00 | 18.18 |
| ATOM | 2025 | CD2 | PHE | A | 786 | 23.034 | −2.254 | 13.419 | 1.00 | 19.90 |
| ATOM | 2026 | CE1 | PHE | A | 786 | 22.570 | −1.986 | 16.125 | 1.00 | 25.35 |
| ATOM | 2027 | CE2 | PHE | A | 786 | 21.972 | −2.916 | 14.004 | 1.00 | 17.12 |
| ATOM | 2028 | CZ | PHE | A | 786 | 21.740 | −2.781 | 15.360 | 1.00 | 16.84 |
| ATOM | 2029 | C | PHE | A | 786 | 26.985 | 0.760 | 13.898 | 1.00 | 9.62 |
| ATOM | 2030 | O | PHE | A | 786 | 27.907 | 0.444 | 14.652 | 1.00 | 10.46 |
| ATOM | 2031 | N | PHE | A | 787 | 27.183 | 1.348 | 12.720 | 1.00 | 8.90 |
| ATOM | 2032 | CA | PHE | A | 787 | 28.531 | 1.680 | 12.273 | 1.00 | 9.83 |
| ATOM | 2033 | CB | PHE | A | 787 | 28.515 | 2.141 | 10.809 | 1.00 | 10.96 |
| ATOM | 2034 | CG | PHE | A | 787 | 28.116 | 1.056 | 9.849 | 1.00 | 8.28 |
| ATOM | 2035 | CD1 | PHE | A | 787 | 28.335 | −0.276 | 10.172 | 1.00 | 10.72 |
| ATOM | 2036 | CD2 | PHE | A | 787 | 27.546 | 1.357 | 8.626 | 1.00 | 11.47 |
| ATOM | 2037 | CE1 | PHE | A | 787 | 27.993 | −1.287 | 9.296 | 1.00 | 12.45 |
| ATOM | 2038 | CE2 | PHE | A | 787 | 27.201 | 0.344 | 7.739 | 1.00 | 12.21 |
| ATOM | 2039 | CZ | PHE | A | 787 | 27.424 | −0.977 | 8.075 | 1.00 | 8.97 |
| ATOM | 2040 | C | PHE | A | 787 | 29.165 | 2.747 | 13.149 | 1.00 | 12.00 |
| ATOM | 2041 | O | PHE | A | 787 | 30.387 | 2.787 | 13.311 | 1.00 | 11.70 |
| ATOM | 2042 | N | ASP | A | 788 | 28.340 | 3.620 | 13.713 | 1.00 | 10.51 |
| ATOM | 2043 | CA | ASP | A | 788 | 28.867 | 4.655 | 14.583 | 1.00 | 13.07 |
| ATOM | 2044 | CB | ASP | A | 788 | 27.770 | 5.654 | 14.953 | 1.00 | 9.28 |
| ATOM | 2045 | CG | ASP | A | 788 | 28.275 | 6.751 | 15.869 | 1.00 | 17.65 |
| ATOM | 2046 | OD1 | ASP | A | 788 | 27.862 | 6.784 | 17.046 | 1.00 | 22.26 |
| ATOM | 2047 | OD2 | ASP | A | 788 | 29.097 | 7.575 | 15.414 | 1.00 | 23.70 |
| ATOM | 2048 | C | ASP | A | 788 | 29.427 | 3.975 | 15.832 | 1.00 | 11.61 |
| ATOM | 2049 | O | ASP | A | 788 | 30.491 | 4.349 | 16.324 | 1.00 | 11.23 |
| ATOM | 2050 | N | GLN | A | 789 | 28.719 | 2.965 | 16.338 | 1.00 | 10.39 |
| ATOM | 2051 | CA | GLN | A | 789 | 29.201 | 2.243 | 17.515 | 1.00 | 10.66 |
| ATOM | 2052 | CB | GLN | A | 789 | 28.185 | 1.199 | 17.979 | 1.00 | 14.04 |
| ATOM | 2053 | CG | GLN | A | 789 | 28.667 | 0.416 | 19.191 | 1.00 | 13.95 |
| ATOM | 2054 | CD | GLN | A | 789 | 27.696 | −0.652 | 19.641 | 1.00 | 13.88 |
| ATOM | 2055 | OE1 | GLN | A | 789 | 26.488 | −0.445 | 19.641 | 1.00 | 10.68 |
| ATOM | 2056 | NE2 | GLN | A | 789 | 28.226 | −1.799 | 20.051 | 1.00 | 10.88 |
| ATOM | 2057 | C | GLN | A | 789 | 30.509 | 1.541 | 17.155 | 1.00 | 12.25 |
| ATOM | 2058 | O | GLN | A | 789 | 31.452 | 1.504 | 17.952 | 1.00 | 12.92 |
| ATOM | 2059 | N | GLY | A | 790 | 30.552 | 0.979 | 15.950 | 1.00 | 12.27 |
| ATOM | 2060 | CA | GLY | A | 790 | 31.746 | 0.292 | 15.493 | 1.00 | 13.69 |
| ATOM | 2061 | C | GLY | A | 790 | 32.948 | 1.218 | 15.519 | 1.00 | 12.41 |
| ATOM | 2062 | O | GLY | A | 790 | 34.050 | 0.805 | 15.885 | 1.00 | 14.19 |
| ATOM | 2063 | N | ASP | A | 791 | 32.743 | 2.476 | 15.135 | 1.00 | 13.24 |
| ATOM | 2064 | CA | ASP | A | 791 | 33.837 | 3.448 | 15.135 | 1.00 | 15.89 |
| ATOM | 2065 | CB | ASP | A | 791 | 33.383 | 4.802 | 14.582 | 1.00 | 11.78 |
| ATOM | 2066 | CG | ASP | A | 791 | 33.022 | 4.748 | 13.108 | 1.00 | 11.72 |
| ATOM | 2067 | OD1 | ASP | A | 791 | 33.525 | 3.853 | 12.401 | 1.00 | 16.07 |
| ATOM | 2068 | OD2 | ASP | A | 791 | 32.249 | 5.619 | 12.657 | 1.00 | 16.56 |
| ATOM | 2069 | C | ASP | A | 791 | 34.350 | 3.653 | 16.554 | 1.00 | 15.06 |
| ATOM | 2070 | O | ASP | A | 791 | 35.555 | 3.751 | 16.794 | 1.00 | 15.97 |
| ATOM | 2071 | N | ARG | A | 792 | 33.423 | 3.723 | 17.496 | 1.00 | 16.00 |
| ATOM | 2072 | CA | ARG | A | 792 | 33.783 | 3.925 | 18.885 | 1.00 | 13.38 |
| ATOM | 2073 | CB | ARG | A | 792 | 32.544 | 4.308 | 19.680 | 1.00 | 15.67 |
| ATOM | 2074 | CG | ARG | A | 792 | 32.056 | 5.689 | 19.304 | 1.00 | 20.98 |
| ATOM | 2075 | CD | ARG | A | 792 | 30.791 | 6.043 | 20.014 | 1.00 | 22.55 |
| ATOM | 2076 | NE | ARG | A | 792 | 30.929 | 6.004 | 21.465 | 1.00 | 21.96 |
| ATOM | 2077 | CZ | ARG | A | 792 | 29.945 | 6.332 | 22.290 | 1.00 | 19.00 |
| ATOM | 2078 | NH1 | ARG | A | 792 | 28.782 | 6.726 | 21.791 | 1.00 | 18.95 |
| ATOM | 2079 | NH2 | ARG | A | 792 | 30.105 | 6.236 | 23.601 | 1.00 | 23.88 |
| ATOM | 2080 | C | ARG | A | 792 | 34.466 | 2.716 | 19.501 | 1.00 | 13.13 |
| ATOM | 2081 | O | ARG | A | 792 | 35.296 | 2.865 | 20.390 | 1.00 | 14.33 |
| ATOM | 2082 | N | GLU | A | 793 | 34.112 | 1.522 | 19.036 | 1.00 | 14.11 |
| ATOM | 2083 | CA | GLU | A | 793 | 34.730 | 0.305 | 19.545 | 1.00 | 12.55 |
| ATOM | 2084 | CB | GLU | A | 793 | 33.969 | −0.932 | 19.051 | 1.00 | 12.93 |
| ATOM | 2085 | CG | GLU | A | 793 | 32.531 | −0.986 | 19.543 | 1.00 | 15.09 |
| ATOM | 2086 | CD | GLU | A | 793 | 31.816 | −2.269 | 19.165 | 1.00 | 17.10 |
| ATOM | 2087 | OE1 | GLU | A | 793 | 32.105 | −2.807 | 18.078 | 1.00 | 14.75 |
| ATOM | 2088 | OE2 | GLU | A | 793 | 30.951 | −2.728 | 19.949 | 1.00 | 13.37 |
| ATOM | 2089 | C | GLU | A | 793 | 36.184 | 0.251 | 19.080 | 1.00 | 13.70 |
| ATOM | 2090 | O | GLU | A | 793 | 37.073 | −0.143 | 19.837 | 1.00 | 14.48 |
| ATOM | 2091 | N | ARG | A | 794 | 36.429 | 0.655 | 17.838 | 1.00 | 12.88 |
| ATOM | 2092 | CA | ARG | A | 794 | 37.792 | 0.647 | 17.318 | 1.00 | 16.50 |
| ATOM | 2093 | CB | ARG | A | 794 | 37.828 | 1.083 | 15.847 | 1.00 | 16.91 |
| ATOM | 2094 | CG | ARG | A | 794 | 37.330 | 0.047 | 14.838 | 1.00 | 16.40 |
| ATOM | 2095 | CD | ARG | A | 794 | 37.399 | 0.609 | 13.422 | 1.00 | 20.95 |
| ATOM | 2096 | NE | ARG | A | 794 | 36.790 | −0.268 | 12.422 | 1.00 | 21.25 |
| ATOM | 2097 | CZ | ARG | A | 794 | 37.323 | −1.407 | 11.996 | 1.00 | 16.41 |
| ATOM | 2098 | NH1 | ARG | A | 794 | 38.486 | −1.822 | 12.480 | 1.00 | 26.91 |
| ATOM | 2099 | NH2 | ARG | A | 794 | 36.691 | −2.134 | 11.082 | 1.00 | 18.30 |
| ATOM | 2100 | C | ARG | A | 794 | 38.648 | 1.603 | 18.141 | 1.00 | 17.48 |
| ATOM | 2101 | O | ARG | A | 794 | 39.781 | 1.287 | 18.503 | 1.00 | 18.61 |
| ATOM | 2102 | N | LYS | A | 795 | 38.087 | 2.769 | 18.443 | 1.00 | 19.00 |
| ATOM | 2103 | CA | LYS | A | 795 | 38.793 | 3.795 | 19.199 | 1.00 | 19.03 |
| ATOM | 2104 | CB | LYS | A | 795 | 38.146 | 5.161 | 18.947 | 1.00 | 22.77 |
| ATOM | 2105 | CG | LYS | A | 795 | 38.839 | 6.312 | 19.661 | 1.00 | 30.54 |
| ATOM | 2106 | CD | LYS | A | 795 | 38.128 | 7.633 | 19.410 | 1.00 | 38.45 |
| ATOM | 2107 | CE | LYS | A | 795 | 38.902 | 8.796 | 20.009 | 1.00 | 34.57 |
| ATOM | 2108 | NZ | LYS | A | 795 | 40.267 | 8.886 | 19.424 | 1.00 | 42.21 |
| ATOM | 2109 | C | LYS | A | 795 | 38.866 | 3.554 | 20.704 | 1.00 | 19.86 |
| ATOM | 2110 | O | LYS | A | 795 | 39.923 | 3.716 | 21.310 | 1.00 | 21.68 |
| ATOM | 2111 | N | GLU | A | 796 | 37.747 | 3.166 | 21.304 | 1.00 | 17.50 |
| ATOM | 2112 | CA | GLU | A | 796 | 37.696 | 2.953 | 22.745 | 1.00 | 18.20 |
| ATOM | 2113 | CB | GLU | A | 796 | 36.286 | 3.272 | 23.255 | 1.00 | 16.63 |
| ATOM | 2114 | CG | GLU | A | 796 | 35.872 | 4.722 | 23.048 | 1.00 | 19.55 |
| ATOM | 2115 | CD | GLU | A | 796 | 34.425 | 4.982 | 23.414 | 1.00 | 17.22 |
| ATOM | 2116 | OE1 | GLU | A | 796 | 33.986 | 4.520 | 24.489 | 1.00 | 17.11 |
| ATOM | 2117 | OE2 | GLU | A | 796 | 33.729 | 5.660 | 22.629 | 1.00 | 17.31 |
| ATOM | 2118 | C | GLU | A | 796 | 38.116 | 1.568 | 23.238 | 1.00 | 18.82 |
| ATOM | 2119 | O | GLU | A | 796 | 38.604 | 1.432 | 24.359 | 1.00 | 19.88 |
| ATOM | 2120 | N | LEU | A | 797 | 37.936 | 0.544 | 22.408 | 1.00 | 17.76 |
| ATOM | 2121 | CA | LEU | A | 797 | 38.279 | −0.817 | 22.809 | 1.00 | 17.55 |
| ATOM | 2122 | CB | LEU | A | 797 | 37.032 | −1.708 | 22.732 | 1.00 | 14.21 |
| ATOM | 2123 | CG | LEU | A | 797 | 35.816 | −1.230 | 23.535 | 1.00 | 21.04 |
| ATOM | 2124 | CD1 | LEU | A | 797 | 34.655 | −2.194 | 23.333 | 1.00 | 19.81 |
| ATOM | 2125 | CD2 | LEU | A | 797 | 36.177 | −1.134 | 25.008 | 1.00 | 22.47 |
| ATOM | 2126 | C | LEU | A | 797 | 39.400 | −1.441 | 21.980 | 1.00 | 18.06 |
| ATOM | 2127 | O | LEU | A | 797 | 39.899 | −2.518 | 22.312 | 1.00 | 18.10 |
| ATOM | 2128 | N | ASN | A | 798 | 39.795 | −0.760 | 20.910 | 1.00 | 17.87 |
| ATOM | 2129 | CA | ASN | A | 798 | 40.846 | −1.244 | 20.016 | 1.00 | 17.14 |
| ATOM | 2130 | CB | ASN | A | 798 | 42.202 | −1.278 | 20.732 | 1.00 | 18.53 |
| ATOM | 2131 | CG | ASN | A | 798 | 43.357 | −1.501 | 19.770 | 1.00 | 19.84 |
| ATOM | 2132 | OD1 | ASN | A | 798 | 43.497 | −0.774 | 18.788 | 1.00 | 22.65 |
| ATOM | 2133 | ND2 | ASN | A | 798 | 44.186 | −2.507 | 20.043 | 1.00 | 17.46 |
| ATOM | 2134 | C | ASN | A | 798 | 40.522 | −2.632 | 19.472 | 1.00 | 19.40 |
| ATOM | 2135 | O | ASN | A | 798 | 41.400 | −3.485 | 19.352 | 1.00 | 18.37 |
| ATOM | 2136 | N | ILE | A | 799 | 39.254 | −2.852 | 19.141 | 1.00 | 18.23 |
| ATOM | 2137 | CA | ILE | A | 799 | 38.816 | −4.134 | 18.603 | 1.00 | 20.72 |
| ATOM | 2138 | CB | ILE | A | 799 | 37.779 | −4.811 | 19.529 | 1.00 | 23.69 |
| ATOM | 2139 | CG2 | ILE | A | 799 | 38.348 | −4.970 | 20.930 | 1.00 | 25.12 |
| ATOM | 2140 | CG1 | ILE | A | 799 | 36.500 | −3.968 | 19.570 | 1.00 | 27.29 |
| ATOM | 2141 | CD1 | ILE | A | 799 | 35.381 | −4.581 | 20.373 | 1.00 | 23.09 |
| ATOM | 2142 | C | ILE | A | 799 | 38.158 | −3.926 | 17.243 | 1.00 | 20.66 |
| ATOM | 2143 | O | ILE | A | 799 | 37.636 | −2.846 | 16.956 | 1.00 | 20.48 |
| ATOM | 2144 | N | GLU | A | 800 | 38.199 | −4.951 | 16.399 | 1.00 | 20.84 |
| ATOM | 2145 | CA | GLU | A | 800 | 37.558 | −4.855 | 15.096 | 1.00 | 19.43 |
| ATOM | 2146 | CB | GLU | A | 800 | 38.162 | −5.856 | 14.109 | 1.00 | 25.89 |
| ATOM | 2147 | CG | GLU | A | 800 | 37.507 | −5.813 | 12.735 | 1.00 | 37.89 |
| ATOM | 2148 | CD | GLU | A | 800 | 38.245 | −6.637 | 11.699 | 1.00 | 48.08 |
| ATOM | 2149 | OE1 | GLU | A | 800 | 38.421 | −7.856 | 11.915 | 1.00 | 47.23 |
| ATOM | 2150 | OE2 | GLU | A | 800 | 38.647 | −6.063 | 10.663 | 1.00 | 52.27 |
| ATOM | 2151 | C | GLU | A | 800 | 36.095 | −5.188 | 15.345 | 1.00 | 16.86 |
| ATOM | 2152 | O | GLU | A | 800 | 35.774 | −6.249 | 15.879 | 1.00 | 20.00 |
| ATOM | 2153 | N | PRO | A | 801 | 35.186 | −4.277 | 14.979 | 1.00 | 16.72 |
| ATOM | 2154 | CD | PRO | A | 801 | 35.413 | −2.971 | 14.340 | 1.00 | 13.94 |
| ATOM | 2155 | CA | PRO | A | 801 | 33.755 | −4.518 | 15.188 | 1.00 | 15.17 |
| ATOM | 2156 | CB | PRO | A | 801 | 33.101 | −3.242 | 14.651 | 1.00 | 11.35 |
| ATOM | 2157 | CG | PRO | A | 801 | 34.189 | −2.204 | 14.760 | 1.00 | 18.39 |
| ATOM | 2158 | C | PRO | A | 801 | 33.249 | −5.750 | 14.446 | 1.00 | 14.88 |
| ATOM | 2159 | O | PRO | A | 801 | 33.836 | −6.168 | 13.437 | 1.00 | 14.57 |
| ATOM | 2160 | N | THR | A | 802 | 32.166 | −6.333 | 14.952 | 1.00 | 13.89 |
| ATOM | 2161 | CA | THR | A | 802 | 31.554 | −7.473 | 14.282 | 1.00 | 13.70 |
| ATOM | 2162 | CB | THR | A | 802 | 30.367 | −8.057 | 15.090 | 1.00 | 18.66 |
| ATOM | 2163 | OG1 | THR | A | 802 | 29.339 | −7.067 | 15.227 | 1.00 | 22.82 |
| ATOM | 2164 | CG2 | THR | A | 802 | 30.824 | −8.491 | 16.475 | 1.00 | 28.52 |
| ATOM | 2165 | C | THR | A | 802 | 31.028 | −6.886 | 12.974 | 1.00 | 13.06 |
| ATOM | 2166 | O | THR | A | 802 | 30.896 | −5.666 | 12.853 | 1.00 | 13.12 |
| ATOM | 2167 | N | ASP | A | 803 | 30.730 | −7.737 | 11.997 | 1.00 | 13.68 |
| ATOM | 2168 | CA | ASP | A | 803 | 30.239 | −7.260 | 10.706 | 1.00 | 13.10 |
| ATOM | 2169 | CB | ASP | A | 803 | 29.784 | −8.436 | 9.840 | 1.00 | 15.94 |
| ATOM | 2170 | CG | ASP | A | 803 | 30.942 | −9.281 | 9.340 | 1.00 | 19.70 |
| ATOM | 2171 | OD1 | ASP | A | 803 | 32.108 | −8.965 | 9.661 | 1.00 | 16.86 |
| ATOM | 2172 | OD2 | ASP | A | 803 | 30.681 | −10.265 | 8.619 | 1.00 | 17.57 |
| ATOM | 2173 | C | ASP | A | 803 | 29.092 | −6.262 | 10.829 | 1.00 | 14.26 |
| ATOM | 2174 | O | ASP | A | 803 | 29.071 | −5.239 | 10.141 | 1.00 | 15.59 |
| ATOM | 2175 | N | LEU | A | 804 | 28.142 | −6.561 | 11.706 | 1.00 | 12.76 |
| ATOM | 2176 | CA | LEU | A | 804 | 26.982 | −5.700 | 11.907 | 1.00 | 13.21 |
| ATOM | 2177 | CB | LEU | A | 804 | 26.116 | −6.251 | 13.042 | 1.00 | 14.65 |
| ATOM | 2178 | CG | LEU | A | 804 | 24.861 | −5.430 | 13.347 | 1.00 | 15.24 |
| ATOM | 2179 | CD1 | LEU | A | 804 | 24.020 | −5.317 | 12.092 | 1.00 | 16.29 |
| ATOM | 2180 | CD2 | LEU | A | 804 | 24.068 | −6.089 | 14.461 | 1.00 | 23.77 |
| ATOM | 2181 | C | LEU | A | 804 | 27.356 | −4.252 | 12.214 | 1.00 | 13.48 |
| ATOM | 2182 | O | LEU | A | 804 | 26.646 | −3.317 | 11.825 | 1.00 | 13.22 |
| ATOM | 2183 | N | MET | A | 805 | 28.476 | −4.067 | 12.904 | 1.00 | 12.28 |
| ATOM | 2184 | CA | MET | A | 805 | 28.909 | −2.725 | 13.276 | 1.00 | 11.75 |
| ATOM | 2185 | CB | MET | A | 805 | 29.141 | −2.671 | 14.790 | 1.00 | 13.28 |
| ATOM | 2186 | CG | MET | A | 805 | 27.859 | −2.865 | 15.589 | 1.00 | 33.42 |
| ATOM | 2187 | SD | MET | A | 805 | 28.125 | −3.166 | 17.338 | 1.00 | 47.91 |
| ATOM | 2188 | CE | MET | A | 805 | 26.447 | −3.244 | 17.925 | 1.00 | 28.62 |
| ATOM | 2189 | C | MET | A | 805 | 30.160 | −2.268 | 12.542 | 1.00 | 11.31 |
| ATOM | 2190 | O | MET | A | 805 | 30.761 | −1.259 | 12.908 | 1.00 | 12.42 |
| ATOM | 2191 | N | ASN | A | 806 | 30.530 | −2.992 | 11.490 | 1.00 | 11.94 |
| ATOM | 2192 | CA | ASN | A | 806 | 31.726 | −2.673 | 10.712 | 1.00 | 13.21 |
| ATOM | 2193 | CB | ASN | A | 806 | 32.537 | −3.954 | 10.471 | 1.00 | 13.42 |
| ATOM | 2194 | CG | ASN | A | 806 | 33.907 | −3.676 | 9.876 | 1.00 | 16.11 |
| ATOM | 2195 | OD1 | ASN | A | 806 | 34.209 | −2.549 | 9.484 | 1.00 | 17.92 |
| ATOM | 2196 | ND2 | ASN | A | 806 | 34.740 | −4.706 | 9.802 | 1.00 | 15.54 |
| ATOM | 2197 | C | ASN | A | 806 | 31.371 | −2.023 | 9.374 | 1.00 | 14.20 |
| ATOM | 2198 | O | ASN | A | 806 | 30.988 | −2.715 | 8.430 | 1.00 | 14.08 |
| ATOM | 2199 | N | ARG | A | 807 | 31.508 | −0.699 | 9.296 | 1.00 | 12.56 |
| ATOM | 2200 | CA | ARG | A | 807 | 31.189 | 0.037 | 8.075 | 1.00 | 14.91 |
| ATOM | 2201 | CB | ARG | A | 807 | 31.410 | 1.543 | 8.275 | 1.00 | 16.99 |
| ATOM | 2202 | CG | ARG | A | 807 | 30.891 | 2.385 | 7.116 | 1.00 | 25.83 |
| ATOM | 2203 | CD | ARG | A | 807 | 31.162 | 3.881 | 7.280 | 1.00 | 30.30 |
| ATOM | 2204 | NE | ARG | A | 807 | 30.520 | 4.470 | 8.457 | 1.00 | 20.02 |
| ATOM | 2205 | CZ | ARG | A | 807 | 31.078 | 4.525 | 9.662 | 1.00 | 16.27 |
| ATOM | 2206 | NH1 | ARG | A | 807 | 32.290 | 4.025 | 9.854 | 1.00 | 20.90 |
| ATOM | 2207 | NH2 | ARG | A | 807 | 30.431 | 5.095 | 10.670 | 1.00 | 15.75 |
| ATOM | 2208 | C | ARG | A | 807 | 32.021 | −0.442 | 6.889 | 1.00 | 13.89 |
| ATOM | 2209 | O | ARG | A | 807 | 31.573 | −0.392 | 5.736 | 1.00 | 13.84 |
| ATOM | 2210 | N | GLU | A | 808 | 33.228 | −0.910 | 7.177 | 1.00 | 11.68 |
| ATOM | 2211 | CA | GLU | A | 808 | 34.122 | −1.397 | 6.137 | 1.00 | 17.06 |
| ATOM | 2212 | CB | GLU | A | 808 | 35.502 | −1.675 | 6.731 | 1.00 | 16.87 |
| ATOM | 2213 | CG | GLU | A | 808 | 36.256 | −0.394 | 7.085 | 1.00 | 22.64 |
| ATOM | 2214 | CD | GLU | A | 808 | 37.556 | −0.645 | 7.819 | 1.00 | 24.85 |
| ATOM | 2215 | OE1 | GLU | A | 808 | 38.293 | −1.576 | 7.430 | 1.00 | 26.32 |
| ATOM | 2216 | OE2 | GLU | A | 808 | 37.846 | 0.100 | 8.780 | 1.00 | 36.15 |
| ATOM | 2217 | C | GLU | A | 808 | 33.565 | −2.641 | 5.461 | 1.00 | 15.58 |
| ATOM | 2218 | O | GLU | A | 808 | 33.929 | −2.956 | 4.329 | 1.00 | 15.69 |
| ATOM | 2219 | N | LYS | A | 809 | 32.677 | −3.343 | 6.156 | 1.00 | 14.84 |
| ATOM | 2220 | CA | LYS | A | 809 | 32.062 | −4.544 | 5.600 | 1.00 | 16.67 |
| ATOM | 2221 | CB | LYS | A | 809 | 32.381 | −5.765 | 6.478 | 1.00 | 12.30 |
| ATOM | 2222 | CG | LYS | A | 809 | 33.845 | −6.184 | 6.405 | 1.00 | 13.87 |
| ATOM | 2223 | CD | LYS | A | 809 | 34.151 | −7.408 | 7.259 | 1.00 | 14.82 |
| ATOM | 2224 | CE | LYS | A | 809 | 33.430 | −8.641 | 6.745 | 1.00 | 25.64 |
| ATOM | 2225 | NZ | LYS | A | 809 | 33.789 | −9.854 | 7.534 | 1.00 | 21.95 |
| ATOM | 2226 | C | LYS | A | 809 | 30.555 | −4.363 | 5.445 | 1.00 | 15.95 |
| ATOM | 2227 | O | LYS | A | 809 | 29.770 | −5.281 | 5.693 | 1.00 | 18.05 |
| ATOM | 2228 | N | LYS | A | 810 | 30.156 | −3.167 | 5.022 | 1.00 | 15.13 |
| ATOM | 2229 | CA | LYS | A | 810 | 28.742 | −2.867 | 4.828 | 1.00 | 16.24 |
| ATOM | 2230 | CB | LYS | A | 810 | 28.549 | −1.383 | 4.494 | 1.00 | 14.35 |
| ATOM | 2231 | CG | LYS | A | 810 | 29.347 | −0.869 | 3.312 | 1.00 | 24.48 |
| ATOM | 2232 | CD | LYS | A | 810 | 29.222 | 0.647 | 3.226 | 1.00 | 29.64 |
| ATOM | 2233 | CE | LYS | A | 810 | 29.994 | 1.224 | 2.049 | 1.00 | 35.68 |
| ATOM | 2234 | NZ | LYS | A | 810 | 29.432 | 0.786 | 0.746 | 1.00 | 42.39 |
| ATOM | 2235 | C | LYS | A | 810 | 28.139 | −3.744 | 3.734 | 1.00 | 14.42 |
| ATOM | 2236 | O | LYS | A | 810 | 26.921 | −3.843 | 3.607 | 1.00 | 14.44 |
| ATOM | 2237 | N | ASN | A | 811 | 28.995 | −4.389 | 2.950 | 1.00 | 13.62 |
| ATOM | 2238 | CA | ASN | A | 811 | 28.511 | −5.270 | 1.893 | 1.00 | 13.60 |
| ATOM | 2239 | CB | ASN | A | 811 | 29.680 | −5.746 | 1.016 | 1.00 | 18.14 |
| ATOM | 2240 | CG | ASN | A | 811 | 30.815 | −6.340 | 1.826 | 1.00 | 21.65 |
| ATOM | 2241 | OD1 | ASN | A | 811 | 30.936 | −7.559 | 1.958 | 1.00 | 17.54 |
| ATOM | 2242 | ND2 | ASN | A | 811 | 31.651 | −5.472 | 2.388 | 1.00 | 13.93 |
| ATOM | 2243 | C | ASN | A | 811 | 27.777 | −6.474 | 2.487 | 1.00 | 14.60 |
| ATOM | 2244 | O | ASN | A | 811 | 27.084 | −7.197 | 1.777 | 1.00 | 13.25 |
| ATOM | 2245 | N | LYS | A | 812 | 27.922 | −6.677 | 3.796 | 1.00 | 14.05 |
| ATOM | 2246 | CA | LYS | A | 812 | 27.279 | −7.797 | 4.478 | 1.00 | 15.17 |
| ATOM | 2247 | CB | LYS | A | 812 | 28.057 | −8.150 | 5.753 | 1.00 | 15.23 |
| ATOM | 2248 | CG | LYS | A | 812 | 29.401 | −8.803 | 5.500 | 1.00 | 20.27 |
| ATOM | 2249 | CD | LYS | A | 812 | 29.198 | −10.137 | 4.817 | 1.00 | 28.44 |
| ATOM | 2250 | CE | LYS | A | 812 | 30.497 | −10.886 | 4.658 | 1.00 | 35.44 |
| ATOM | 2251 | NZ | LYS | A | 812 | 30.257 | −12.223 | 4.048 | 1.00 | 38.51 |
| ATOM | 2252 | C | LYS | A | 812 | 25.818 | −7.542 | 4.859 | 1.00 | 15.78 |
| ATOM | 2253 | O | LYS | A | 812 | 25.109 | −8.475 | 5.258 | 1.00 | 13.98 |
| ATOM | 2254 | N | ILE | A | 813 | 25.366 | −6.297 | 4.724 | 1.00 | 14.64 |
| ATOM | 2255 | CA | ILE | A | 813 | 24.012 | −5.964 | 5.144 | 1.00 | 14.83 |
| ATOM | 2256 | CB | ILE | A | 813 | 23.770 | −4.460 | 5.007 | 1.00 | 15.21 |
| ATOM | 2257 | CG2 | ILE | A | 813 | 22.264 | −4.158 | 5.091 | 1.00 | 15.81 |
| ATOM | 2258 | CG1 | ILE | A | 813 | 24.616 | −3.740 | 6.073 | 1.00 | 13.99 |
| ATOM | 2259 | CD1 | ILE | A | 813 | 24.445 | −2.265 | 6.070 | 1.00 | 27.43 |
| ATOM | 2260 | C | ILE | A | 813 | 22.862 | −6.792 | 4.546 | 1.00 | 14.67 |
| ATOM | 2261 | O | ILE | A | 813 | 22.042 | −7.309 | 5.270 | 1.00 | 13.44 |
| ATOM | 2262 | N | PRO | A | 814 | 22.807 | −6.936 | 3.224 | 1.00 | 12.07 |
| ATOM | 2263 | CD | PRO | A | 814 | 23.603 | −6.274 | 2.191 | 1.00 | 14.86 |
| ATOM | 2264 | CA | PRO | A | 814 | 21.709 | −7.724 | 2.623 | 1.00 | 14.39 |
| ATOM | 2265 | CB | PRO | A | 814 | 22.060 | −7.714 | 1.152 | 1.00 | 13.45 |
| ATOM | 2266 | CG | PRO | A | 814 | 22.693 | −6.351 | 1.001 | 1.00 | 14.67 |
| ATOM | 2267 | C | PRO | A | 814 | 21.605 | −9.144 | 3.185 | 1.00 | 11.82 |
| ATOM | 2268 | O | PRO | A | 814 | 20.525 | −9.603 | 3.570 | 1.00 | 12.87 |
| ATOM | 2269 | N | SER | A | 815 | 22.730 | −9.846 | 3.215 | 1.00 | 11.71 |
| ATOM | 2270 | CA | SER | A | 815 | 22.730 | −11.205 | 3.729 | 1.00 | 13.13 |
| ATOM | 2271 | CB | SER | A | 815 | 24.102 | −11.863 | 3.526 | 1.00 | 14.66 |
| ATOM | 2272 | OG | SER | A | 815 | 25.124 | −11.185 | 4.233 | 1.00 | 27.24 |
| ATOM | 2273 | C | SER | A | 815 | 22.366 | −11.220 | 5.212 | 1.00 | 14.09 |
| ATOM | 2274 | O | SER | A | 815 | 21.677 | −12.120 | 5.682 | 1.00 | 14.61 |
| ATOM | 2275 | N | MET | A | 816 | 22.809 | −10.211 | 5.937 | 1.00 | 14.82 |
| ATOM | 2276 | CA | MET | A | 816 | 22.498 | −10.197 | 7.353 | 1.00 | 14.08 |
| ATOM | 2277 | CB | MET | A | 816 | 23.449 | −9.254 | 8.079 | 1.00 | 16.95 |
| ATOM | 2278 | CG | MET | A | 816 | 24.829 | −9.872 | 8.232 | 1.00 | 19.08 |
| ATOM | 2279 | SD | MET | A | 816 | 26.019 | −8.905 | 9.168 | 1.00 | 23.16 |
| ATOM | 2280 | CE | MET | A | 816 | 25.502 | −9.310 | 10.812 | 1.00 | 29.41 |
| ATOM | 2281 | C | MET | A | 816 | 21.057 | −9.838 | 7.628 | 1.00 | 12.78 |
| ATOM | 2282 | O | MET | A | 816 | 20.477 | −10.281 | 8.613 | 1.00 | 13.06 |
| ATOM | 2283 | N | GLN | A | 817 | 20.464 | −9.010 | 6.765 | 1.00 | 11.62 |
| ATOM | 2284 | CA | GLN | A | 817 | 19.061 | −8.637 | 6.915 | 1.00 | 11.33 |
| ATOM | 2285 | CB | GLN | A | 817 | 18.666 | −7.547 | 5.899 | 1.00 | 12.94 |
| ATOM | 2286 | CG | GLN | A | 817 | 19.186 | −6.179 | 6.283 | 1.00 | 11.95 |
| ATOM | 2287 | CD | GLN | A | 817 | 18.469 | −5.624 | 7.500 | 1.00 | 17.94 |
| ATOM | 2288 | OE1 | GLN | A | 817 | 18.544 | −6.183 | 8.595 | 1.00 | 34.56 |
| ATOM | 2289 | NE2 | GLN | A | 817 | 17.759 | −4.523 | 7.309 | 1.00 | 34.13 |
| ATOM | 2290 | C | GLN | A | 817 | 18.202 | −9.882 | 6.714 | 1.00 | 13.31 |
| ATOM | 2291 | O | GLN | A | 817 | 17.254 | −10.116 | 7.472 | 1.00 | 10.14 |
| ATOM | 2292 | N | VAL | A | 818 | 18.537 | −10.698 | 5.715 | 1.00 | 12.18 |
| ATOM | 2293 | CA | VAL | A | 818 | 17.774 | −11.918 | 5.479 | 1.00 | 11.53 |
| ATOM | 2294 | CB | VAL | A | 818 | 18.252 | −12.659 | 4.210 | 1.00 | 13.56 |
| ATOM | 2295 | CG1 | VAL | A | 818 | 17.500 | −13.972 | 4.063 | 1.00 | 10.58 |
| ATOM | 2296 | CG2 | VAL | A | 818 | 18.032 | −11.781 | 2.982 | 1.00 | 17.92 |
| ATOM | 2297 | C | VAL | A | 818 | 17.903 | −12.858 | 6.678 | 1.00 | 11.51 |
| ATOM | 2298 | O | VAL | A | 818 | 16.930 | −13.494 | 7.090 | 1.00 | 12.55 |
| ATOM | 2299 | N | GLY | A | 819 | 19.110 | −12.934 | 7.231 | 1.00 | 11.44 |
| ATOM | 2300 | CA | GLY | A | 819 | 19.356 | −13.791 | 8.376 | 1.00 | 12.74 |
| ATOM | 2301 | C | GLY | A | 819 | 18.556 | −13.335 | 9.579 | 1.00 | 13.96 |
| ATOM | 2302 | O | GLY | A | 819 | 18.068 | −14.149 | 10.360 | 1.00 | 13.49 |
| ATOM | 2303 | N | PHE | A | 820 | 18.430 | −12.022 | 9.725 | 1.00 | 14.20 |
| ATOM | 2304 | CA | PHE | A | 820 | 17.682 | −11.426 | 10.825 | 1.00 | 11.70 |
| ATOM | 2305 | CB | PHE | A | 820 | 17.927 | −9.913 | 10.833 | 1.00 | 11.32 |
| ATOM | 2306 | CG | PHE | A | 820 | 17.122 | −9.167 | 11.858 | 1.00 | 9.99 |
| ATOM | 2307 | CD1 | PHE | A | 820 | 17.268 | −9.436 | 13.209 | 1.00 | 23.52 |
| ATOM | 2308 | CD2 | PHE | A | 820 | 16.225 | −8.184 | 11.466 | 1.00 | 17.26 |
| ATOM | 2309 | CE1 | PHE | A | 820 | 16.529 | −8.740 | 14.151 | 1.00 | 19.87 |
| ATOM | 2310 | CE2 | PHE | A | 820 | 15.485 | −7.488 | 12.403 | 1.00 | 16.59 |
| ATOM | 2311 | CZ | PHE | A | 820 | 15.640 | −7.767 | 13.746 | 1.00 | 17.66 |
| ATOM | 2312 | C | PHE | A | 820 | 16.191 | −11.731 | 10.651 | 1.00 | 12.70 |
| ATOM | 2313 | O | PHE | A | 820 | 15.498 | −12.103 | 11.604 | 1.00 | 10.72 |
| ATOM | 2314 | N | ILE | A | 821 | 15.697 | −11.571 | 9.429 | 1.00 | 10.04 |
| ATOM | 2315 | CA | ILE | A | 821 | 14.298 | −11.850 | 9.150 | 1.00 | 9.29 |
| ATOM | 2316 | CB | ILE | A | 821 | 13.957 | −11.540 | 7.679 | 1.00 | 10.14 |
| ATOM | 2317 | CG2 | ILE | A | 821 | 12.617 | −12.166 | 7.307 | 1.00 | 12.87 |
| ATOM | 2318 | CG1 | ILE | A | 821 | 13.931 | −10.022 | 7.479 | 1.00 | 12.03 |
| ATOM | 2319 | CD1 | ILE | A | 821 | 13.795 | −9.580 | 6.037 | 1.00 | 14.77 |
| ATOM | 2320 | C | ILE | A | 821 | 13.991 | −13.311 | 9.462 | 1.00 | 11.67 |
| ATOM | 2321 | O | ILE | A | 821 | 13.012 | −13.617 | 10.140 | 1.00 | 11.27 |
| ATOM | 2322 | N | ASP | A | 822 | 14.841 | −14.215 | 8.985 | 1.00 | 11.97 |
| ATOM | 2323 | CA | ASP | A | 822 | 14.634 | −15.637 | 9.237 | 1.00 | 14.30 |
| ATOM | 2324 | CB | ASP | A | 822 | 15.630 | −16.479 | 8.428 | 1.00 | 17.59 |
| ATOM | 2325 | CG | ASP | A | 822 | 15.317 | −16.499 | 6.942 | 1.00 | 17.92 |
| ATOM | 2326 | OD1 | ASP | A | 822 | 14.177 | −16.153 | 6.564 | 1.00 | 15.98 |
| ATOM | 2327 | OD2 | ASP | A | 822 | 16.212 | −16.880 | 6.152 | 1.00 | 13.97 |
| ATOM | 2328 | C | ASP | A | 822 | 14.759 | −16.021 | 10.714 | 1.00 | 15.42 |
| ATOM | 2329 | O | ASP | A | 822 | 13.876 | −16.671 | 11.275 | 1.00 | 16.53 |
| ATOM | 2330 | N | ALA | A | 823 | 15.849 | −15.608 | 11.345 | 1.00 | 13.55 |
| ATOM | 2331 | CA | ALA | A | 823 | 16.101 | −15.971 | 12.737 | 1.00 | 15.03 |
| ATOM | 2332 | CB | ALA | A | 823 | 17.580 | −15.756 | 13.058 | 1.00 | 13.84 |
| ATOM | 2333 | C | ALA | A | 823 | 15.254 | −15.299 | 13.810 | 1.00 | 15.13 |
| ATOM | 2334 | O | ALA | A | 823 | 14.907 | −15.928 | 14.810 | 1.00 | 17.91 |
| ATOM | 2335 | N | ILE | A | 824 | 14.914 | −14.032 | 13.606 | 1.00 | 13.91 |
| ATOM | 2336 | CA | ILE | A | 824 | 14.169 | −13.281 | 14.611 | 1.00 | 14.87 |
| ATOM | 2337 | CB | ILE | A | 824 | 14.929 | −11.971 | 14.966 | 1.00 | 16.51 |
| ATOM | 2338 | CG2 | ILE | A | 824 | 14.085 | −11.106 | 15.891 | 1.00 | 11.92 |
| ATOM | 2339 | CG1 | ILE | A | 824 | 16.291 | −12.293 | 15.589 | 1.00 | 19.03 |
| ATOM | 2340 | CD1 | ILE | A | 824 | 16.211 | −12.966 | 16.938 | 1.00 | 17.79 |
| ATOM | 2341 | C | ILE | A | 824 | 12.736 | −12.872 | 14.271 | 1.00 | 15.38 |
| ATOM | 2342 | O | ILE | A | 824 | 11.817 | −13.051 | 15.077 | 1.00 | 15.58 |
| ATOM | 2343 | N | CYS | A | 825 | 12.548 | −12.332 | 13.074 | 1.00 | 11.66 |
| ATOM | 2344 | CA | CYS | A | 825 | 11.248 | −11.798 | 12.682 | 1.00 | 10.83 |
| ATOM | 2345 | CB | CYS | A | 825 | 11.470 | −10.717 | 11.629 | 1.00 | 14.06 |
| ATOM | 2346 | SG | CYS | A | 825 | 12.684 | −9.498 | 12.156 | 1.00 | 12.72 |
| ATOM | 2347 | C | CYS | A | 825 | 10.094 | −12.685 | 12.231 | 1.00 | 12.83 |
| ATOM | 2348 | O | CYS | A | 825 | 9.003 | −12.606 | 12.792 | 1.00 | 11.78 |
| ATOM | 2349 | N | LEU | A | 826 | 10.312 | −13.508 | 11.213 | 1.00 | 10.64 |
| ATOM | 2350 | CA | LEU | A | 826 | 9.230 | −14.343 | 10.699 | 1.00 | 12.29 |
| ATOM | 2351 | CB | LEU | A | 826 | 9.759 | −15.291 | 9.621 | 1.00 | 17.45 |
| ATOM | 2352 | CG | LEU | A | 826 | 10.132 | −14.585 | 8.314 | 1.00 | 14.44 |
| ATOM | 2353 | CD1 | LEU | A | 826 | 10.756 | −15.585 | 7.355 | 1.00 | 19.69 |
| ATOM | 2354 | CD2 | LEU | A | 826 | 8.884 | −13.965 | 7.693 | 1.00 | 17.13 |
| ATOM | 2355 | C | LEU | A | 826 | 8.428 | −15.131 | 11.735 | 1.00 | 11.75 |
| ATOM | 2356 | O | LEU | A | 826 | 7.194 | −15.094 | 11.728 | 1.00 | 12.87 |
| ATOM | 2357 | N | GLN | A | 827 | 9.115 | −15.842 | 12.620 | 1.00 | 9.56 |
| ATOM | 2358 | CA | GLN | A | 827 | 8.426 | −16.637 | 13.632 | 1.00 | 10.96 |
| ATOM | 2359 | CB | GLN | A | 827 | 9.438 | −17.368 | 14.507 | 1.00 | 9.95 |
| ATOM | 2360 | CG | GLN | A | 827 | 10.207 | −18.465 | 13.805 | 1.00 | 14.60 |
| ATOM | 2361 | CD | GLN | A | 827 | 11.365 | −18.949 | 14.646 | 1.00 | 27.21 |
| ATOM | 2362 | OE1 | GLN | A | 827 | 12.420 | −18.312 | 14.695 | 1.00 | 20.68 |
| ATOM | 2363 | NE2 | GLN | A | 827 | 11.169 | −20.068 | 15.335 | 1.00 | 24.19 |
| ATOM | 2364 | C | GLN | A | 827 | 7.516 | −15.798 | 14.524 | 1.00 | 13.14 |
| ATOM | 2365 | O | GLN | A | 827 | 6.428 | −16.235 | 14.906 | 1.00 | 13.21 |
| ATOM | 2366 | N | LEU | A | 828 | 7.966 | −14.596 | 14.862 | 1.00 | 10.02 |
| ATOM | 2367 | CA | LEU | A | 828 | 7.182 | −13.713 | 15.720 | 1.00 | 10.78 |
| ATOM | 2368 | CB | LEU | A | 828 | 8.037 | −12.521 | 16.166 | 1.00 | 9.67 |
| ATOM | 2369 | CG | LEU | A | 828 | 7.315 | −11.431 | 16.966 | 1.00 | 14.41 |
| ATOM | 2370 | CD1 | LEU | A | 828 | 6.653 | −12.048 | 18.185 | 1.00 | 16.13 |
| ATOM | 2371 | CD2 | LEU | A | 828 | 8.300 | −10.351 | 17.389 | 1.00 | 14.03 |
| ATOM | 2372 | C | LEU | A | 828 | 5.902 | −13.218 | 15.036 | 1.00 | 11.55 |
| ATOM | 2373 | O | LEU | A | 828 | 4.814 | −13.290 | 15.609 | 1.00 | 12.50 |
| ATOM | 2374 | N | TYR | A | 829 | 6.024 | −12.717 | 13.812 | 1.00 | 10.38 |
| ATOM | 2375 | CA | TYR | A | 829 | 4.851 | −12.217 | 13.109 | 1.00 | 11.38 |
| ATOM | 2376 | CB | TYR | A | 829 | 5.286 | −11.392 | 11.892 | 1.00 | 9.07 |
| ATOM | 2377 | CG | TYR | A | 829 | 5.967 | −10.112 | 12.324 | 1.00 | 13.32 |
| ATOM | 2378 | CD1 | TYR | A | 829 | 5.298 | −9.189 | 13.118 | 1.00 | 8.62 |
| ATOM | 2379 | CE1 | TYR | A | 829 | 5.926 | −8.037 | 13.569 | 1.00 | 10.58 |
| ATOM | 2380 | CD2 | TYR | A | 829 | 7.290 | −9.849 | 11.989 | 1.00 | 10.78 |
| ATOM | 2381 | CE2 | TYR | A | 829 | 7.930 | −8.699 | 12.440 | 1.00 | 8.02 |
| ATOM | 2382 | CZ | TYR | A | 829 | 7.241 | −7.800 | 13.228 | 1.00 | 8.64 |
| ATOM | 2383 | OH | TYR | A | 829 | 7.867 | −6.659 | 13.688 | 1.00 | 10.63 |
| ATOM | 2384 | C | TYR | A | 829 | 3.908 | −13.349 | 12.731 | 1.00 | 12.07 |
| ATOM | 2385 | O | TYR | A | 829 | 2.704 | −13.140 | 12.594 | 1.00 | 14.07 |
| ATOM | 2386 | N | GLU | A | 830 | 4.452 | −14.555 | 12.585 | 1.00 | 14.19 |
| ATOM | 2387 | CA | GLU | A | 830 | 3.614 | −15.709 | 12.274 | 1.00 | 14.53 |
| ATOM | 2388 | CB | GLU | A | 830 | 4.469 | −16.919 | 11.881 | 1.00 | 15.21 |
| ATOM | 2389 | CG | GLU | A | 830 | 5.196 | −16.751 | 10.557 | 1.00 | 18.48 |
| ATOM | 2390 | CD | GLU | A | 830 | 6.057 | −17.948 | 10.208 | 1.00 | 27.35 |
| ATOM | 2391 | OE1 | GLU | A | 830 | 6.422 | −18.701 | 11.133 | 1.00 | 25.55 |
| ATOM | 2392 | OE2 | GLU | A | 830 | 6.380 | −18.127 | 9.014 | 1.00 | 25.64 |
| ATOM | 2393 | C | GLU | A | 830 | 2.806 | −16.027 | 13.531 | 1.00 | 15.34 |
| ATOM | 2394 | O | GLU | A | 830 | 1.594 | −16.245 | 13.462 | 1.00 | 15.05 |
| ATOM | 2395 | N | ALA | A | 831 | 3.484 | −16.037 | 14.679 | 1.00 | 12.88 |
| ATOM | 2396 | CA | ALA | A | 831 | 2.836 | −16.319 | 15.958 | 1.00 | 13.60 |
| ATOM | 2397 | CB | ALA | A | 831 | 3.877 | −16.341 | 17.080 | 1.00 | 14.75 |
| ATOM | 2398 | C | ALA | A | 831 | 1.764 | −15.279 | 16.264 | 1.00 | 14.36 |
| ATOM | 2399 | O | ALA | A | 831 | 0.666 | −15.617 | 16.714 | 1.00 | 13.70 |
| ATOM | 2400 | N | LEU | A | 832 | 2.085 | −14.010 | 16.026 | 1.00 | 11.70 |
| ATOM | 2401 | CA | LEU | A | 832 | 1.135 | −12.935 | 16.276 | 1.00 | 12.90 |
| ATOM | 2402 | CB | LEU | A | 832 | 1.800 | −11.577 | 16.030 | 1.00 | 12.02 |
| ATOM | 2403 | CG | LEU | A | 832 | 0.935 | −10.334 | 16.256 | 1.00 | 13.07 |
| ATOM | 2404 | CD1 | LEU | A | 832 | 0.367 | −10.354 | 17.670 | 1.00 | 16.97 |
| ATOM | 2405 | CD2 | LEU | A | 832 | 1.764 | −9.076 | 16.018 | 1.00 | 12.70 |
| ATOM | 2406 | C | LEU | A | 832 | −0.093 | −13.098 | 15.382 | 1.00 | 13.07 |
| ATOM | 2407 | O | LEU | A | 832 | −1.221 | −12.851 | 15.811 | 1.00 | 12.63 |
| ATOM | 2408 | N | THR | A | 833 | 0.125 | −13.522 | 14.140 | 1.00 | 10.33 |
| ATOM | 2409 | CA | THR | A | 833 | −0.985 | −13.720 | 13.213 | 1.00 | 11.65 |
| ATOM | 2410 | CB | THR | A | 833 | −0.471 | −14.019 | 11.798 | 1.00 | 15.54 |
| ATOM | 2411 | OG1 | THR | A | 833 | 0.250 | −12.881 | 11.309 | 1.00 | 13.44 |
| ATOM | 2412 | CG2 | THR | A | 833 | −1.631 | −14.319 | 10.856 | 1.00 | 20.09 |
| ATOM | 2413 | C | THR | A | 833 | −1.856 | −14.875 | 13.709 | 1.00 | 13.24 |
| ATOM | 2414 | O | THR | A | 833 | −3.070 | −14.879 | 13.512 | 1.00 | 14.62 |
| ATOM | 2415 | N | HIS | A | 834 | −1.233 | −15.855 | 14.358 | 1.00 | 12.04 |
| ATOM | 2416 | CA | HIS | A | 834 | −1.974 | −16.988 | 14.901 | 1.00 | 15.35 |
| ATOM | 2417 | CB | HIS | A | 834 | −1.010 | −18.045 | 15.451 | 1.00 | 17.31 |
| ATOM | 2418 | CG | HIS | A | 834 | −1.686 | −19.161 | 16.174 | 1.00 | 21.70 |
| ATOM | 2419 | CD2 | HIS | A | 834 | −2.005 | −20.422 | 15.792 | 1.00 | 27.35 |
| ATOM | 2420 | ND1 | HIS | A | 834 | −2.158 | −19.040 | 17.471 | 1.00 | 27.23 |
| ATOM | 2421 | CE1 | HIS | A | 834 | −2.728 | −20.162 | 17.843 | 1.00 | 22.73 |
| ATOM | 2422 | NE2 | HIS | A | 834 | −2.650 | −21.026 | 16.836 | 1.00 | 26.03 |
| ATOM | 2423 | C | HIS | A | 834 | −2.907 | −16.495 | 16.011 | 1.00 | 16.43 |
| ATOM | 2424 | O | HIS | A | 834 | −4.061 | −16.917 | 16.101 | 1.00 | 16.32 |
| ATOM | 2425 | N | VAL | A | 835 | −2.402 | −15.600 | 16.852 | 1.00 | 14.39 |
| ATOM | 2426 | CA | VAL | A | 835 | −3.201 | −15.042 | 17.940 | 1.00 | 16.17 |
| ATOM | 2427 | CB | VAL | A | 835 | −2.329 | −14.177 | 18.879 | 1.00 | 20.06 |
| ATOM | 2428 | CG1 | VAL | A | 835 | −3.203 | −13.433 | 19.878 | 1.00 | 26.18 |
| ATOM | 2429 | CG2 | VAL | A | 835 | −1.336 | −15.067 | 19.616 | 1.00 | 22.85 |
| ATOM | 2430 | C | VAL | A | 835 | −4.345 | −14.196 | 17.376 | 1.00 | 18.30 |
| ATOM | 2431 | O | VAL | A | 835 | −5.462 | −14.219 | 17.899 | 1.00 | 16.68 |
| ATOM | 2432 | N | SER | A | 836 | −4.061 | −13.461 | 16.302 | 1.00 | 15.55 |
| ATOM | 2433 | CA | SER | A | 836 | −5.060 | −12.620 | 15.646 | 1.00 | 15.62 |
| ATOM | 2434 | CB | SER | A | 836 | −5.081 | −11.222 | 16.267 | 1.00 | 18.44 |
| ATOM | 2435 | OG | SER | A | 836 | −6.092 | −10.430 | 15.665 | 1.00 | 24.05 |
| ATOM | 2436 | C | SER | A | 836 | −4.744 | −12.516 | 14.153 | 1.00 | 15.03 |
| ATOM | 2437 | O | SER | A | 836 | −3.754 | −11.901 | 13.759 | 1.00 | 14.82 |
| ATOM | 2438 | N | GLU | A | 837 | −5.596 | −13.123 | 13.334 | 1.00 | 16.29 |
| ATOM | 2439 | CA | GLU | A | 837 | −5.423 | −13.139 | 11.886 | 1.00 | 16.40 |
| ATOM | 2440 | CB | GLU | A | 837 | −6.578 | −13.922 | 11.244 | 1.00 | 19.29 |
| ATOM | 2441 | CG | GLU | A | 837 | −6.524 | −14.017 | 9.722 | 1.00 | 30.56 |
| ATOM | 2442 | CD | GLU | A | 837 | −5.259 | −14.685 | 9.213 | 1.00 | 40.78 |
| ATOM | 2443 | OE1 | GLU | A | 837 | −4.925 | −15.785 | 9.704 | 1.00 | 33.63 |
| ATOM | 2444 | OE2 | GLU | A | 837 | −4.603 | −14.112 | 8.316 | 1.00 | 45.55 |
| ATOM | 2445 | C | GLU | A | 837 | −5.324 | −11.749 | 11.256 | 1.00 | 14.00 |
| ATOM | 2446 | O | GLU | A | 837 | −4.672 | −11.574 | 10.227 | 1.00 | 16.56 |
| ATOM | 2447 | N | ASP | A | 838 | −5.967 | −10.762 | 11.871 | 1.00 | 14.38 |
| ATOM | 2448 | CA | ASP | A | 838 | −5.938 | −9.402 | 11.349 | 1.00 | 12.78 |
| ATOM | 2449 | CB | ASP | A | 838 | −6.990 | −8.547 | 12.060 | 1.00 | 12.80 |
| ATOM | 2450 | CG | ASP | A | 838 | −8.410 | −8.929 | 11.669 | 1.00 | 31.66 |
| ATOM | 2451 | OD1 | ASP | A | 838 | −8.770 | −8.750 | 10.487 | 1.00 | 25.15 |
| ATOM | 2452 | OD2 | ASP | A | 838 | −9.162 | −9.414 | 12.540 | 1.00 | 29.94 |
| ATOM | 2453 | C | ASP | A | 838 | −4.558 | −8.751 | 11.468 | 1.00 | 11.81 |
| ATOM | 2454 | O | ASP | A | 838 | −4.355 | −7.625 | 11.017 | 1.00 | 12.87 |
| ATOM | 2455 | N | CYS | A | 839 | −3.610 | −9.456 | 12.080 | 1.00 | 14.34 |
| ATOM | 2456 | CA | CYS | A | 839 | −2.254 | −8.929 | 12.203 | 1.00 | 14.23 |
| ATOM | 2457 | CB | CYS | A | 839 | −1.619 | −9.354 | 13.533 | 1.00 | 12.27 |
| ATOM | 2458 | SG | CYS | A | 839 | −2.115 | −8.349 | 14.969 | 1.00 | 12.53 |
| ATOM | 2459 | C | CYS | A | 839 | −1.412 | −9.440 | 11.038 | 1.00 | 15.74 |
| ATOM | 2460 | O | CYS | A | 839 | −0.222 | −9.142 | 10.941 | 1.00 | 13.52 |
| ATOM | 2461 | N | PHE | A | 840 | −2.044 | −10.209 | 10.152 | 1.00 | 14.15 |
| ATOM | 2462 | CA | PHE | A | 840 | −1.364 | −10.765 | 8.983 | 1.00 | 14.85 |
| ATOM | 2463 | CB | PHE | A | 840 | −2.370 | −11.451 | 8.052 | 1.00 | 18.01 |
| ATOM | 2464 | CG | PHE | A | 840 | −1.760 | −11.926 | 6.764 | 1.00 | 16.92 |
| ATOM | 2465 | CD1 | PHE | A | 840 | −0.886 | −13.001 | 6.751 | 1.00 | 13.52 |
| ATOM | 2466 | CD2 | PHE | A | 840 | −2.010 | −11.257 | 5.575 | 1.00 | 19.38 |
| ATOM | 2467 | CE1 | PHE | A | 840 | −0.269 | −13.398 | 5.575 | 1.00 | 23.30 |
| ATOM | 2468 | CE2 | PHE | A | 840 | −1.397 | −11.649 | 4.396 | 1.00 | 23.75 |
| ATOM | 2469 | CZ | PHE | A | 840 | −0.525 | −12.720 | 4.397 | 1.00 | 22.76 |
| ATOM | 2470 | C | PHE | A | 840 | −0.541 | −9.768 | 8.155 | 1.00 | 13.70 |
| ATOM | 2471 | O | PHE | A | 840 | 0.546 | −10.105 | 7.678 | 1.00 | 13.33 |
| ATOM | 2472 | N | PRO | A | 841 | −1.049 | −8.537 | 7.958 | 1.00 | 12.97 |
| ATOM | 2473 | CD | PRO | A | 841 | −2.369 | −8.002 | 8.339 | 1.00 | 14.47 |
| ATOM | 2474 | CA | PRO | A | 841 | −0.294 | −7.557 | 7.168 | 1.00 | 12.84 |
| ATOM | 2475 | CB | PRO | A | 841 | −1.127 | −6.287 | 7.312 | 1.00 | 14.37 |
| ATOM | 2476 | CG | PRO | A | 841 | −2.533 | −6.826 | 7.391 | 1.00 | 14.58 |
| ATOM | 2477 | C | PRO | A | 841 | 1.151 | −7.367 | 7.630 | 1.00 | 14.54 |
| ATOM | 2478 | O | PRO | A | 841 | 2.034 | −7.065 | 6.826 | 1.00 | 13.75 |
| ATOM | 2479 | N | LEU | A | 842 | 1.393 | −7.530 | 8.926 | 1.00 | 11.66 |
| ATOM | 2480 | CA | LEU | A | 842 | 2.747 | −7.384 | 9.444 | 1.00 | 12.35 |
| ATOM | 2481 | CB | LEU | A | 842 | 2.732 | −7.430 | 10.977 | 1.00 | 11.85 |
| ATOM | 2482 | CG | LEU | A | 842 | 2.039 | −6.228 | 11.624 | 1.00 | 12.78 |
| ATOM | 2483 | CD1 | LEU | A | 842 | 1.900 | −6.434 | 13.131 | 1.00 | 14.23 |
| ATOM | 2484 | CD2 | LEU | A | 842 | 2.837 | −4.975 | 11.321 | 1.00 | 14.25 |
| ATOM | 2485 | C | LEU | A | 842 | 3.613 | −8.513 | 8.880 | 1.00 | 13.95 |
| ATOM | 2486 | O | LEU | A | 842 | 4.741 | −8.290 | 8.444 | 1.00 | 13.84 |
| ATOM | 2487 | N | LEU | A | 843 | 3.067 | −9.727 | 8.882 | 1.00 | 11.90 |
| ATOM | 2488 | CA | LEU | A | 843 | 3.779 | −10.889 | 8.359 | 1.00 | 11.64 |
| ATOM | 2489 | CB | LEU | A | 843 | 2.995 | −12.161 | 8.670 | 1.00 | 10.07 |
| ATOM | 2490 | CG | LEU | A | 843 | 3.532 | −13.468 | 8.084 | 1.00 | 9.50 |
| ATOM | 2491 | CD1 | LEU | A | 843 | 4.940 | −13.735 | 8.588 | 1.00 | 13.64 |
| ATOM | 2492 | CD2 | LEU | A | 843 | 2.597 | −14.608 | 8.466 | 1.00 | 12.37 |
| ATOM | 2493 | C | LEU | A | 843 | 3.971 | −10.751 | 6.847 | 1.00 | 11.74 |
| ATOM | 2494 | O | LEU | A | 843 | 5.054 | −11.003 | 6.319 | 1.00 | 13.14 |
| ATOM | 2495 | N | ASP | A | 844 | 2.909 | −10.342 | 6.160 | 1.00 | 13.31 |
| ATOM | 2496 | CA | ASP | A | 844 | 2.943 | −10.154 | 4.711 | 1.00 | 14.45 |
| ATOM | 2497 | CB | ASP | A | 844 | 1.573 | −9.639 | 4.245 | 1.00 | 18.19 |
| ATOM | 2498 | CG | ASP | A | 844 | 1.354 | −9.771 | 2.739 | 1.00 | 13.51 |
| ATOM | 2499 | OD1 | ASP | A | 844 | 2.141 | −10.459 | 2.057 | 1.00 | 18.86 |
| ATOM | 2500 | OD2 | ASP | A | 844 | 0.367 | −9.187 | 2.243 | 1.00 | 17.91 |
| ATOM | 2501 | C | ASP | A | 844 | 4.053 | −9.153 | 4.366 | 1.00 | 13.87 |
| ATOM | 2502 | O | ASP | A | 844 | 4.854 | −9.386 | 3.460 | 1.00 | 13.07 |
| ATOM | 2503 | N | GLY | A | 845 | 4.100 | −8.046 | 5.104 | 1.00 | 12.67 |
| ATOM | 2504 | CA | GLY | A | 845 | 5.115 | −7.035 | 4.862 | 1.00 | 11.69 |
| ATOM | 2505 | C | GLY | A | 845 | 6.529 | −7.540 | 5.097 | 1.00 | 11.05 |
| ATOM | 2506 | O | GLY | A | 845 | 7.447 | −7.240 | 4.330 | 1.00 | 12.51 |
| ATOM | 2507 | N | CYS | A | 846 | 6.704 | −8.314 | 6.161 | 1.00 | 10.28 |
| ATOM | 2508 | CA | CYS | A | 846 | 8.005 | −8.882 | 6.507 | 1.00 | 10.93 |
| ATOM | 2509 | CB | CYS | A | 846 | 7.889 | −9.643 | 7.831 | 1.00 | 14.58 |
| ATOM | 2510 | SG | CYS | A | 846 | 9.443 | −10.273 | 8.485 | 1.00 | 12.82 |
| ATOM | 2511 | C | CYS | A | 846 | 8.497 | −9.821 | 5.398 | 1.00 | 11.92 |
| ATOM | 2512 | O | CYS | A | 846 | 9.661 | −9.762 | 4.985 | 1.00 | 11.24 |
| ATOM | 2513 | N | ARG | A | 847 | 7.605 | −10.683 | 4.914 | 1.00 | 12.77 |
| ATOM | 2514 | CA | ARG | A | 847 | 7.953 | −11.618 | 3.853 | 1.00 | 13.72 |
| ATOM | 2515 | CB | ARG | A | 847 | 6.762 | −12.527 | 3.535 | 1.00 | 11.63 |
| ATOM | 2516 | CG | ARG | A | 847 | 6.457 | −13.545 | 4.619 | 1.00 | 13.10 |
| ATOM | 2517 | CD | ARG | A | 847 | 5.180 | −14.308 | 4.316 | 1.00 | 17.35 |
| ATOM | 2518 | NE | ARG | A | 847 | 4.978 | −15.402 | 5.262 | 1.00 | 19.23 |
| ATOM | 2519 | CZ | ARG | A | 847 | 3.840 | −16.077 | 5.389 | 1.00 | 21.47 |
| ATOM | 2520 | NH1 | ARG | A | 847 | 2.796 | −15.769 | 4.632 | 1.00 | 24.29 |
| ATOM | 2521 | NH2 | ARG | A | 847 | 3.750 | −17.059 | 6.275 | 1.00 | 27.21 |
| ATOM | 2522 | C | ARG | A | 847 | 8.371 | −10.870 | 2.592 | 1.00 | 13.53 |
| ATOM | 2523 | O | ARG | A | 847 | 9.320 | −11.265 | 1.906 | 1.00 | 13.20 |
| ATOM | 2524 | N | LYS | A | 848 | 7.659 | −9.793 | 2.283 | 1.00 | 12.35 |
| ATOM | 2525 | CA | LYS | A | 848 | 7.977 | −9.007 | 1.099 | 1.00 | 14.65 |
| ATOM | 2526 | CB | LYS | A | 848 | 6.925 | −7.915 | 0.880 | 1.00 | 16.22 |
| ATOM | 2527 | CG | LYS | A | 848 | 5.567 | −8.447 | 0.413 | 1.00 | 14.77 |
| ATOM | 2528 | CD | LYS | A | 848 | 4.579 | −7.308 | 0.195 | 1.00 | 19.65 |
| ATOM | 2529 | CE | LYS | A | 848 | 3.210 | −7.817 | −0.206 | 1.00 | 19.42 |
| ATOM | 2530 | NZ | LYS | A | 848 | 2.214 | −6.709 | −0.212 | 1.00 | 23.11 |
| ATOM | 2531 | C | LYS | A | 848 | 9.364 | −8.392 | 1.229 | 1.00 | 14.80 |
| ATOM | 2532 | O | LYS | A | 848 | 10.112 | −8.319 | 0.253 | 1.00 | 13.12 |
| ATOM | 2533 | N | ASN | A | 849 | 9.713 | −7.953 | 2.435 | 1.00 | 13.42 |
| ATOM | 2534 | CA | ASN | A | 849 | 11.034 | −7.369 | 2.648 | 1.00 | 13.07 |
| ATOM | 2535 | CB | ASN | A | 849 | 11.086 | −6.646 | 3.995 | 1.00 | 13.67 |
| ATOM | 2536 | CG | ASN | A | 849 | 10.412 | −5.288 | 3.950 | 1.00 | 17.85 |
| ATOM | 2537 | OD1 | ASN | A | 849 | 9.863 | −4.821 | 4.949 | 1.00 | 16.87 |
| ATOM | 2538 | ND2 | ASN | A | 849 | 10.461 | −4.638 | 2.790 | 1.00 | 13.21 |
| ATOM | 2539 | C | ASN | A | 849 | 12.122 | −8.434 | 2.568 | 1.00 | 12.00 |
| ATOM | 2540 | O | ASN | A | 849 | 13.252 | −8.148 | 2.165 | 1.00 | 11.88 |
| ATOM | 2541 | N | ARG | A | 850 | 11.797 | −9.667 | 2.945 | 1.00 | 11.53 |
| ATOM | 2542 | CA | ARG | A | 850 | 12.793 | −10.729 | 2.860 | 1.00 | 11.98 |
| ATOM | 2543 | CB | ARG | A | 850 | 12.285 | −12.037 | 3.473 | 1.00 | 14.13 |
| ATOM | 2544 | CG | ARG | A | 850 | 13.373 | −13.110 | 3.508 | 1.00 | 13.55 |
| ATOM | 2545 | CD | ARG | A | 850 | 12.838 | −14.511 | 3.781 | 1.00 | 18.02 |
| ATOM | 2546 | NE | ARG | A | 850 | 13.930 | −15.457 | 4.001 | 1.00 | 16.95 |
| ATOM | 2547 | CZ | ARG | A | 850 | 14.743 | −15.919 | 3.052 | 1.00 | 14.40 |
| ATOM | 2548 | NH1 | ARG | A | 850 | 14.595 | −15.533 | 1.790 | 1.00 | 16.03 |
| ATOM | 2549 | NH2 | ARG | A | 850 | 15.727 | −16.754 | 3.373 | 1.00 | 12.51 |
| ATOM | 2550 | C | ARG | A | 850 | 13.138 | −10.972 | 1.399 | 1.00 | 13.81 |
| ATOM | 2551 | O | ARG | A | 850 | 14.304 | −11.151 | 1.054 | 1.00 | 15.14 |
| ATOM | 2552 | N | GLN | A | 851 | 12.118 | −10.985 | 0.542 | 1.00 | 16.03 |
| ATOM | 2553 | CA | GLN | A | 851 | 12.317 | −11.193 | −0.892 | 1.00 | 15.42 |
| ATOM | 2554 | CB | GLN | A | 851 | 10.969 | −11.156 | −1.622 | 1.00 | 17.30 |
| ATOM | 2555 | CG | GLN | A | 851 | 11.039 | −11.399 | −3.127 | 1.00 | 30.46 |
| ATOM | 2556 | CD | GLN | A | 851 | 11.475 | −10.174 | −3.912 | 1.00 | 34.81 |
| ATOM | 2557 | OE1 | GLN | A | 851 | 10.862 | −9.110 | −3.814 | 1.00 | 37.41 |
| ATOM | 2558 | NE2 | GLN | A | 851 | 12.534 | −10.320 | −4.704 | 1.00 | 40.89 |
| ATOM | 2559 | C | GLN | A | 851 | 13.229 | −10.109 | −1.436 | 1.00 | 16.49 |
| ATOM | 2560 | O | GLN | A | 851 | 14.151 | −10.371 | −2.208 | 1.00 | 15.70 |
| ATOM | 2561 | N | LYS | A | 852 | 12.953 | −8.867 | −1.014 | 1.00 | 15.86 |
| ATOM | 2562 | CA | LYS | A | 852 | 13.705 | −7.685 | −1.428 | 1.00 | 16.63 |
| ATOM | 2563 | CB | LYS | A | 852 | 13.065 | −6.405 | −0.838 | 1.00 | 13.58 |
| ATOM | 2564 | CG | LYS | A | 852 | 11.802 | −6.008 | −1.561 | 1.00 | 18.37 |
| ATOM | 2565 | CD | LYS | A | 852 | 12.183 | −5.582 | −2.958 | 1.00 | 31.95 |
| ATOM | 2566 | CE | LYS | A | 852 | 11.002 | −5.087 | −3.751 | 1.00 | 37.83 |
| ATOM | 2567 | NZ | LYS | A | 852 | 11.421 | −4.599 | −5.099 | 1.00 | 41.92 |
| ATOM | 2568 | C | LYS | A | 852 | 15.175 | −7.754 | −1.037 | 1.00 | 15.46 |
| ATOM | 2569 | O | LYS | A | 852 | 16.055 | −7.542 | −1.874 | 1.00 | 14.58 |
| ATOM | 2570 | N | TRP | A | 853 | 15.446 | −8.063 | 0.225 | 1.00 | 12.99 |
| ATOM | 2571 | CA | TRP | A | 853 | 16.821 | −8.168 | 0.705 | 1.00 | 12.72 |
| ATOM | 2572 | CB | TRP | A | 853 | 16.843 | −8.299 | 2.228 | 1.00 | 11.94 |
| ATOM | 2573 | CG | TRP | A | 853 | 16.640 | −7.011 | 2.957 | 1.00 | 11.40 |
| ATOM | 2574 | CD2 | TRP | A | 853 | 17.485 | −5.864 | 2.913 | 1.00 | 14.74 |
| ATOM | 2575 | CE2 | TRP | A | 853 | 16.929 | −4.888 | 3.763 | 1.00 | 12.41 |
| ATOM | 2576 | CE3 | TRP | A | 853 | 18.675 | −5.554 | 2.236 | 1.00 | 10.90 |
| ATOM | 2577 | CD1 | TRP | A | 853 | 15.618 | −6.701 | 3.813 | 1.00 | 11.02 |
| ATOM | 2578 | NE1 | TRP | A | 853 | 15.781 | −5.429 | 4.303 | 1.00 | 14.15 |
| ATOM | 2579 | CZ2 | TRP | A | 853 | 17.502 | −3.638 | 3.958 | 1.00 | 12.03 |
| ATOM | 2580 | CZ3 | TRP | A | 853 | 19.246 | −4.312 | 2.427 | 1.00 | 13.77 |
| ATOM | 2581 | CH2 | TRP | A | 853 | 18.663 | −3.370 | 3.280 | 1.00 | 16.08 |
| ATOM | 2582 | C | TRP | A | 853 | 17.543 | −9.357 | 0.074 | 1.00 | 13.55 |
| ATOM | 2583 | O | TRP | A | 853 | 18.722 | −9.265 | −0.282 | 1.00 | 13.19 |
| ATOM | 2584 | N | GLN | A | 854 | 16.836 | −10.472 | −0.062 | 1.00 | 12.06 |
| ATOM | 2585 | CA | GLN | A | 854 | 17.427 | −11.667 | −0.657 | 1.00 | 13.49 |
| ATOM | 2586 | CB | GLN | A | 854 | 16.426 | −12.822 | −0.630 | 1.00 | 13.86 |
| ATOM | 2587 | CG | GLN | A | 854 | 16.934 | −14.128 | −1.242 | 1.00 | 15.91 |
| ATOM | 2588 | CD | GLN | A | 854 | 18.267 | −14.585 | −0.665 | 1.00 | 23.66 |
| ATOM | 2589 | OE1 | GLN | A | 854 | 18.544 | −14.404 | 0.520 | 1.00 | 20.44 |
| ATOM | 2590 | NE2 | GLN | A | 854 | 19.091 | −15.199 | −1.504 | 1.00 | 26.84 |
| ATOM | 2591 | C | GLN | A | 854 | 17.869 | −11.400 | −2.094 | 1.00 | 13.14 |
| ATOM | 2592 | O | GLN | A | 854 | 18.920 | −11.880 | −2.524 | 1.00 | 15.55 |
| ATOM | 2593 | N | ALA | A | 855 | 17.069 | −10.635 | −2.832 | 1.00 | 14.63 |
| ATOM | 2594 | CA | ALA | A | 855 | 17.408 | −10.318 | −4.219 | 1.00 | 15.08 |
| ATOM | 2595 | CB | ALA | A | 855 | 16.264 | −9.563 | −4.884 | 1.00 | 17.06 |
| ATOM | 2596 | C | ALA | A | 855 | 18.697 | −9.504 | −4.303 | 1.00 | 16.23 |
| ATOM | 2597 | O | ALA | A | 855 | 19.439 | −9.611 | −5.278 | 1.00 | 17.06 |
| ATOM | 2598 | N | LEU | A | 856 | 18.959 | −8.689 | −3.287 | 1.00 | 15.11 |
| ATOM | 2599 | CA | LEU | A | 856 | 20.178 | −7.889 | −3.261 | 1.00 | 17.23 |
| ATOM | 2600 | CB | LEU | A | 856 | 20.031 | −6.715 | −2.284 | 1.00 | 11.53 |
| ATOM | 2601 | CG | LEU | A | 856 | 18.968 | −5.676 | −2.643 | 1.00 | 15.60 |
| ATOM | 2602 | CD1 | LEU | A | 856 | 18.996 | −4.549 | −1.634 | 1.00 | 19.73 |
| ATOM | 2603 | CD2 | LEU | A | 856 | 19.226 | −5.145 | −4.047 | 1.00 | 12.95 |
| ATOM | 2604 | C | LEU | A | 856 | 21.362 | −8.754 | −2.835 | 1.00 | 17.35 |
| ATOM | 2605 | O | LEU | A | 856 | 22.478 | −8.601 | −3.337 | 1.00 | 17.30 |
| ATOM | 2606 | N | ALA | A | 857 | 21.109 | −9.674 | −1.911 | 1.00 | 16.52 |
| ATOM | 2607 | CA | ALA | A | 857 | 22.156 | −10.545 | −1.396 | 1.00 | 17.82 |
| ATOM | 2608 | CB | ALA | A | 857 | 21.708 | −11.155 | −0.079 | 1.00 | 15.78 |
| ATOM | 2609 | C | ALA | A | 857 | 22.616 | −11.651 | −2.339 | 1.00 | 19.95 |
| ATOM | 2610 | O | ALA | A | 857 | 23.781 | −12.048 | −2.305 | 1.00 | 21.30 |
| ATOM | 2611 | N | GLU | A | 858 | 21.715 | −12.144 | −3.181 | 1.00 | 19.75 |
| ATOM | 2612 | CA | GLU | A | 858 | 22.059 | −13.233 | −4.091 | 1.00 | 23.14 |
| ATOM | 2613 | CB | GLU | A | 858 | 20.798 | −14.008 | −4.475 | 1.00 | 28.23 |
| ATOM | 2614 | CG | GLU | A | 858 | 19.735 | −13.155 | −5.139 | 1.00 | 35.46 |
| ATOM | 2615 | CD | GLU | A | 858 | 18.516 | −13.957 | −5.548 | 1.00 | 41.30 |
| ATOM | 2616 | OE1 | GLU | A | 858 | 17.928 | −14.635 | −4.679 | 1.00 | 46.06 |
| ATOM | 2617 | OE2 | GLU | A | 858 | 18.144 | −13.905 | −6.739 | 1.00 | 53.40 |
| ATOM | 2618 | C | GLU | A | 858 | 22.773 | −12.775 | −5.356 | 1.00 | 24.66 |
| ATOM | 2619 | O | GLU | A | 858 | 22.769 | −11.561 | −5.640 | 1.00 | 27.06 |
| ATOM | 2620 | OXT | GLU | A | 858 | 23.319 | −13.654 | −6.053 | 1.00 | 33.90 |
| TER | 2621 | | GLU | A | 858 | | | | | |
| ATOM | 2622 | O | HOH | W | 1 | 14.659 | 12.373 | 18.930 | 1.00 | 11.90 |
| ATOM | 2623 | O | HOH | W | 2 | 9.029 | 0.616 | 15.467 | 1.00 | 15.60 |
| ATOM | 2624 | O | HOH | W | 3 | 17.339 | −0.310 | 24.269 | 1.00 | 9.87 |
| ATOM | 2625 | O | HOH | W | 4 | 17.787 | 8.492 | 22.312 | 1.00 | 9.91 |
| ATOM | 2626 | O | HOH | W | 5 | 22.322 | 0.101 | 41.405 | 1.00 | 10.58 |
| ATOM | 2627 | O | HOH | W | 6 | 7.037 | −4.929 | 2.938 | 1.00 | 15.52 |
| ATOM | 2628 | O | HOH | W | 7 | 24.844 | 0.942 | 17.890 | 1.00 | 12.19 |
| ATOM | 2629 | O | HOH | W | 8 | 17.812 | −5.285 | 21.940 | 1.00 | 13.09 |
| ATOM | 2630 | O | HOH | W | 9 | 19.355 | −1.244 | 21.749 | 1.00 | 11.09 |
| ATOM | 2631 | O | HOH | W | 10 | 11.998 | −15.929 | 13.123 | 1.00 | 15.08 |
| ATOM | 2632 | O | HOH | W | 11 | 17.595 | 6.868 | 5.858 | 1.00 | 17.40 |
| ATOM | 2633 | O | HOH | W | 12 | 20.522 | −8.124 | 28.490 | 1.00 | 16.30 |
| ATOM | 2634 | O | HOH | W | 13 | 7.122 | 8.426 | 30.706 | 1.00 | 12.74 |
| ATOM | 2635 | O | HOH | W | 14 | 14.281 | −0.736 | 24.882 | 1.00 | 10.20 |
| ATOM | 2636 | O | HOH | W | 15 | 27.778 | −4.812 | 7.714 | 1.00 | 14.34 |
| ATOM | 2637 | O | HOH | W | 16 | 45.697 | −5.856 | 19.373 | 1.00 | 13.79 |
| ATOM | 2638 | O | HOH | W | 17 | 32.028 | 0.851 | 11.859 | 1.00 | 12.49 |
| ATOM | 2639 | O | HOH | W | 18 | 12.716 | −7.833 | 22.260 | 1.00 | 11.62 |
| ATOM | 2640 | O | HOH | W | 19 | 20.770 | −7.019 | 24.953 | 1.00 | 13.66 |
| ATOM | 2641 | O | HOH | W | 20 | 9.679 | −8.387 | 30.457 | 1.00 | 18.04 |
| ATOM | 2642 | O | HOH | W | 21 | 20.996 | −1.566 | 19.584 | 1.00 | 15.27 |
| ATOM | 2643 | O | HOH | W | 22 | 5.367 | −8.570 | 29.548 | 1.00 | 17.36 |
| ATOM | 2644 | O | HOH | W | 23 | 25.138 | −9.337 | 1.725 | 1.00 | 17.63 |
| ATOM | 2645 | O | HOH | W | 24 | −9.020 | −0.116 | 43.708 | 1.00 | 15.91 |
| ATOM | 2646 | O | HOH | W | 25 | 22.657 | −6.174 | 29.194 | 1.00 | 17.18 |
| ATOM | 2647 | O | HOH | W | 26 | −4.280 | 19.557 | 21.192 | 1.00 | 19.85 |
| ATOM | 2648 | O | HOH | W | 27 | 14.289 | 6.604 | 13.852 | 1.00 | 14.85 |
| ATOM | 2649 | O | HOH | W | 28 | 11.411 | −1.302 | 13.935 | 1.00 | 12.80 |
| ATOM | 2650 | O | HOH | W | 29 | 11.700 | 13.882 | 15.200 | 1.00 | 14.97 |
| ATOM | 2651 | O | HOH | W | 30 | 18.410 | 10.076 | 12.196 | 1.00 | 19.59 |
| ATOM | 2652 | O | HOH | W | 31 | 26.709 | 7.571 | 23.378 | 1.00 | 17.44 |
| ATOM | 2653 | O | HOH | W | 32 | 25.420 | 5.805 | 17.773 | 1.00 | 17.40 |
| ATOM | 2654 | O | HOH | W | 33 | 11.087 | 10.589 | 10.070 | 1.00 | 17.81 |
| ATOM | 2655 | O | HOH | W | 34 | 11.500 | −19.954 | 26.231 | 1.00 | 16.07 |
| ATOM | 2656 | O | HOH | W | 35 | −5.204 | −5.042 | 34.106 | 1.00 | 17.24 |
| ATOM | 2657 | O | HOH | W | 36 | 1.941 | 9.940 | 36.428 | 1.00 | 13.95 |
| ATOM | 2658 | O | HOH | W | 37 | 23.916 | 7.238 | 15.924 | 1.00 | 14.00 |
| ATOM | 2659 | O | HOH | W | 38 | 4.522 | 21.473 | 19.911 | 1.00 | 20.60 |
| ATOM | 2660 | O | HOH | W | 39 | 7.654 | 32.374 | 24.586 | 1.00 | 19.77 |
| ATOM | 2661 | O | HOH | W | 40 | −11.105 | 8.617 | 12.732 | 1.00 | 20.19 |
| ATOM | 2662 | O | HOH | W | 41 | 14.266 | 14.289 | 20.855 | 1.00 | 16.50 |
| ATOM | 2663 | O | HOH | W | 42 | 27.797 | −9.140 | 13.072 | 1.00 | 17.28 |
| ATOM | 2664 | O | HOH | W | 43 | 13.361 | −5.781 | 20.636 | 1.00 | 17.62 |
| ATOM | 2665 | O | HOH | W | 44 | 9.439 | 17.952 | 22.920 | 1.00 | 20.34 |
| ATOM | 2666 | O | HOH | W | 45 | 13.506 | −19.339 | 23.851 | 1.00 | 17.51 |
| ATOM | 2667 | O | HOH | W | 46 | −9.608 | −2.484 | 44.794 | 1.00 | 18.03 |
| ATOM | 2668 | O | HOH | W | 47 | 19.179 | −3.517 | 23.479 | 1.00 | 14.63 |
| ATOM | 2669 | O | HOH | W | 48 | 20.825 | −1.771 | 7.357 | 1.00 | 15.56 |
| ATOM | 2670 | O | HOH | W | 49 | 3.953 | −2.401 | 8.103 | 1.00 | 15.98 |
| ATOM | 2671 | O | HOH | W | 50 | 5.164 | 2.828 | 12.389 | 1.00 | 17.57 |
| ATOM | 2672 | O | HOH | W | 51 | 22.475 | −6.004 | 8.390 | 1.00 | 20.31 |
| ATOM | 2673 | O | HOH | W | 52 | −1.505 | −7.713 | 3.922 | 1.00 | 20.93 |
| ATOM | 2674 | O | HOH | W | 53 | 16.030 | 13.344 | 15.066 | 1.00 | 22.86 |
| ATOM | 2675 | O | HOH | W | 54 | 3.120 | 33.114 | 17.327 | 1.00 | 18.81 |
| ATOM | 2676 | O | HOH | W | 55 | 6.517 | 19.676 | 19.112 | 1.00 | 20.43 |
| ATOM | 2677 | O | HOH | W | 56 | 27.812 | 5.941 | 11.269 | 1.00 | 19.39 |
| ATOM | 2678 | O | HOH | W | 57 | 14.704 | 9.138 | 12.638 | 1.00 | 17.37 |
| ATOM | 2679 | O | HOH | W | 58 | 10.813 | −19.129 | 28.734 | 1.00 | 23.50 |
| ATOM | 2680 | O | HOH | W | 59 | 9.603 | −13.958 | 1.403 | 1.00 | 20.05 |
| ATOM | 2681 | O | HOH | W | 60 | 46.209 | −3.550 | 17.835 | 1.00 | 18.22 |
| ATOM | 2682 | O | HOH | W | 61 | 24.760 | −2.396 | 10.006 | 1.00 | 20.80 |
| ATOM | 2683 | O | HOH | W | 62 | 11.077 | 3.087 | 12.585 | 1.00 | 21.05 |
| ATOM | 2684 | O | HOH | W | 63 | 10.534 | −10.726 | 28.998 | 1.00 | 21.18 |
| ATOM | 2685 | O | HOH | W | 64 | −0.961 | −11.064 | 33.440 | 1.00 | 18.63 |
| ATOM | 2686 | O | HOH | W | 65 | 20.890 | 0.577 | 15.388 | 1.00 | 19.77 |
| ATOM | 2687 | O | HOH | W | 66 | 21.685 | 13.866 | 20.281 | 1.00 | 24.54 |
| ATOM | 2688 | O | HOH | W | 67 | −5.012 | −3.878 | 8.180 | 1.00 | 21.24 |
| ATOM | 2689 | O | HOH | W | 68 | −10.443 | 9.371 | 15.194 | 1.00 | 19.03 |
| ATOM | 2690 | O | HOH | W | 69 | 26.635 | 8.578 | 39.152 | 1.00 | 19.48 |
| ATOM | 2691 | O | HOH | W | 70 | 21.837 | −4.044 | 23.094 | 1.00 | 14.46 |
| ATOM | 2692 | O | HOH | W | 71 | 18.979 | 0.534 | 3.275 | 1.00 | 19.32 |
| ATOM | 2693 | O | HOH | W | 72 | −7.687 | −4.529 | 35.484 | 1.00 | 19.76 |
| ATOM | 2694 | O | HOH | W | 73 | −4.441 | 9.972 | 35.483 | 1.00 | 21.75 |
| ATOM | 2695 | O | HOH | W | 74 | 22.147 | 0.611 | 18.233 | 1.00 | 21.06 |
| ATOM | 2696 | O | HOH | W | 75 | 25.625 | 7.694 | 19.834 | 1.00 | 17.79 |
| ATOM | 2697 | O | HOH | W | 76 | −9.716 | 14.941 | 11.027 | 1.00 | 20.22 |
| ATOM | 2698 | O | HOH | W | 77 | 14.015 | −1.710 | 16.866 | 1.00 | 19.03 |
| ATOM | 2699 | O | HOH | W | 78 | 21.861 | −3.953 | 20.354 | 1.00 | 17.68 |
| ATOM | 2700 | O | HOH | W | 79 | 21.854 | −11.471 | 10.659 | 1.00 | 21.05 |
| ATOM | 2701 | O | HOH | W | 80 | 15.795 | −5.944 | −4.295 | 1.00 | 17.34 |
| ATOM | 2702 | O | HOH | W | 81 | 26.427 | −5.333 | 20.748 | 1.00 | 28.65 |
| ATOM | 2703 | O | HOH | W | 82 | 0.806 | 16.637 | 36.547 | 1.00 | 22.11 |
| ATOM | 2704 | O | HOH | W | 83 | 28.883 | 5.909 | 33.300 | 1.00 | 35.99 |
| ATOM | 2705 | O | HOH | W | 84 | 0.615 | −5.836 | 36.804 | 1.00 | 21.63 |
| ATOM | 2706 | O | HOH | W | 85 | 14.416 | −12.696 | −3.692 | 1.00 | 22.36 |
| ATOM | 2707 | O | HOH | W | 86 | −4.841 | 7.644 | 43.980 | 1.00 | 19.92 |
| ATOM | 2708 | O | HOH | W | 87 | −6.059 | 5.564 | 10.293 | 1.00 | 22.49 |
| ATOM | 2709 | O | HOH | W | 88 | 7.210 | 1.031 | 13.002 | 1.00 | 19.78 |
| ATOM | 2710 | O | HOH | W | 89 | −5.786 | −6.329 | 9.023 | 1.00 | 20.32 |
| ATOM | 2711 | O | HOH | W | 90 | 19.117 | 4.909 | 35.027 | 1.00 | 17.30 |
| ATOM | 2712 | O | HOH | W | 91 | 10.592 | 13.017 | 38.293 | 1.00 | 23.66 |
| ATOM | 2713 | O | HOH | W | 92 | −9.801 | 5.234 | 16.132 | 1.00 | 20.64 |
| ATOM | 2714 | O | HOH | W | 93 | −6.370 | −17.172 | 24.540 | 1.00 | 23.65 |
| ATOM | 2715 | O | HOH | W | 94 | −6.833 | 1.342 | 45.207 | 1.00 | 17.92 |
| ATOM | 2716 | O | HOH | W | 95 | 33.715 | −7.279 | 11.000 | 1.00 | 17.67 |
| ATOM | 2717 | O | HOH | W | 96 | 5.722 | 31.659 | 29.252 | 1.00 | 19.35 |
| ATOM | 2718 | O | HOH | W | 97 | 14.155 | −2.890 | 10.083 | 1.00 | 16.30 |
| ATOM | 2719 | O | HOH | W | 98 | 19.709 | −5.898 | 20.414 | 1.00 | 27.45 |
| ATOM | 2720 | O | HOH | W | 99 | 4.418 | −3.958 | 2.629 | 1.00 | 23.70 |
| ATOM | 2721 | O | HOH | W | 100 | 5.739 | 5.419 | 41.566 | 1.00 | 26.30 |
| ATOM | 2722 | O | HOH | W | 101 | 1.882 | −10.334 | 12.547 | 1.00 | 21.56 |
| ATOM | 2723 | O | HOH | W | 102 | 1.506 | 9.558 | 7.516 | 1.00 | 21.76 |
| ATOM | 2724 | O | HOH | W | 103 | 23.211 | 1.757 | 4.698 | 1.00 | 23.21 |
| ATOM | 2725 | O | HOH | W | 104 | 36.282 | 2.363 | 9.440 | 1.00 | 27.13 |
| ATOM | 2726 | O | HOH | W | 105 | 27.600 | −11.267 | 2.472 | 1.00 | 18.59 |
| ATOM | 2727 | O | HOH | W | 106 | 16.305 | 16.757 | 22.862 | 1.00 | 25.14 |
| ATOM | 2728 | O | HOH | W | 107 | 21.223 | −0.443 | 4.807 | 1.00 | 20.51 |
| ATOM | 2729 | O | HOH | W | 108 | 12.939 | 13.840 | 12.680 | 1.00 | 26.10 |
| ATOM | 2730 | O | HOH | W | 109 | 10.191 | −9.652 | 24.549 | 1.00 | 17.97 |
| ATOM | 2731 | O | HOH | W | 110 | 9.527 | −19.990 | 11.160 | 1.00 | 34.52 |
| ATOM | 2732 | O | HOH | W | 111 | 2.300 | −4.117 | 6.982 | 1.00 | 21.40 |
| ATOM | 2733 | O | HOH | W | 112 | 31.807 | 5.599 | 25.384 | 1.00 | 19.28 |
| ATOM | 2734 | O | HOH | W | 113 | 11.858 | 5.625 | 12.858 | 1.00 | 18.80 |
| ATOM | 2735 | O | HOH | W | 114 | 2.975 | 3.851 | 3.583 | 1.00 | 28.02 |
| ATOM | 2736 | O | HOH | W | 115 | 22.328 | −3.325 | 9.278 | 1.00 | 18.13 |
| ATOM | 2737 | O | HOH | W | 116 | 7.581 | 21.278 | 17.210 | 1.00 | 26.33 |
| ATOM | 2738 | O | HOH | W | 117 | −8.959 | 18.330 | 19.702 | 1.00 | 25.41 |
| ATOM | 2739 | O | HOH | W | 118 | −7.913 | −3.625 | 46.688 | 1.00 | 16.16 |
| ATOM | 2740 | O | HOH | W | 119 | 29.369 | −4.122 | 35.016 | 1.00 | 21.35 |
| ATOM | 2741 | O | HOH | W | 120 | −10.313 | 4.301 | 39.676 | 1.00 | 20.84 |
| ATOM | 2742 | O | HOH | W | 121 | 36.288 | −8.163 | 10.383 | 1.00 | 29.41 |
| ATOM | 2743 | O | HOH | W | 122 | −5.305 | 0.154 | 7.066 | 1.00 | 32.03 |
| ATOM | 2744 | O | HOH | W | 123 | 15.892 | 9.345 | 6.948 | 1.00 | 31.98 |
| ATOM | 2745 | O | HOH | W | 124 | −7.934 | −11.981 | 26.594 | 1.00 | 24.88 |
| ATOM | 2746 | O | HOH | W | 125 | −7.700 | −14.762 | 14.417 | 1.00 | 27.16 |
| ATOM | 2747 | O | HOH | W | 126 | 7.924 | 19.345 | 32.073 | 1.00 | 27.10 |
| ATOM | 2748 | O | HOH | W | 127 | 22.963 | −15.271 | −0.194 | 1.00 | 34.57 |
| ATOM | 2749 | O | HOH | W | 128 | 14.018 | 4.418 | 0.344 | 1.00 | 27.94 |
| ATOM | 2750 | O | HOH | W | 129 | 13.334 | 16.377 | 17.301 | 1.00 | 27.05 |
| ATOM | 2751 | O | HOH | W | 130 | 30.641 | −11.391 | 0.176 | 1.00 | 21.79 |
| ATOM | 2752 | O | HOH | W | 131 | −5.143 | −9.116 | 6.397 | 1.00 | 26.22 |
| ATOM | 2753 | O | HOH | W | 132 | 44.452 | 2.118 | 22.189 | 1.00 | 25.35 |
| ATOM | 2754 | O | HOH | W | 133 | −0.942 | −8.268 | 32.526 | 1.00 | 21.73 |
| ATOM | 2755 | O | HOH | W | 134 | 32.286 | 8.100 | 17.531 | 1.00 | 30.18 |
| ATOM | 2756 | O | HOH | W | 135 | 4.471 | −22.268 | 17.254 | 1.00 | 22.18 |
| ATOM | 2757 | O | HOH | W | 136 | 20.964 | −7.154 | 10.051 | 1.00 | 23.34 |
| ATOM | 2758 | O | HOH | W | 137 | −6.984 | 26.107 | 22.746 | 1.00 | 26.33 |
| ATOM | 2759 | O | HOH | W | 138 | 17.856 | 14.348 | 24.807 | 1.00 | 20.33 |
| ATOM | 2760 | O | HOH | W | 139 | 10.038 | −5.546 | 41.193 | 1.00 | 26.62 |
| ATOM | 2761 | O | HOH | W | 140 | 13.989 | 13.837 | 16.687 | 1.00 | 27.53 |
| ATOM | 2762 | O | HOH | W | 141 | 23.629 | −13.084 | 9.502 | 1.00 | 28.21 |
| ATOM | 2763 | O | HOH | W | 142 | 31.635 | 7.713 | 14.307 | 1.00 | 30.30 |
| ATOM | 2764 | O | HOH | W | 143 | 6.811 | −2.344 | −0.868 | 1.00 | 24.47 |
| ATOM | 2765 | O | HOH | W | 144 | 7.278 | −9.981 | 30.756 | 1.00 | 27.03 |
| ATOM | 2766 | O | HOH | W | 145 | −6.981 | 7.089 | 6.281 | 1.00 | 26.94 |
| ATOM | 2767 | O | HOH | W | 146 | −8.635 | 18.558 | 29.861 | 1.00 | 27.91 |
| ATOM | 2768 | O | HOH | W | 147 | 30.525 | −1.680 | 34.334 | 1.00 | 24.09 |
| ATOM | 2769 | O | HOH | W | 148 | 2.251 | −13.867 | 2.121 | 1.00 | 25.49 |
| ATOM | 2770 | O | HOH | W | 149 | −8.063 | −9.740 | 29.191 | 1.00 | 24.34 |
| ATOM | 2771 | O | HOH | W | 150 | −1.333 | 15.279 | 37.020 | 1.00 | 28.81 |
| ATOM | 2772 | O | HOH | W | 151 | 18.429 | 15.610 | 21.408 | 1.00 | 22.50 |
| ATOM | 2773 | O | HOH | W | 152 | 13.471 | −4.264 | 12.528 | 1.00 | 19.38 |
| ATOM | 2774 | O | HOH | W | 153 | 18.621 | −17.600 | 7.222 | 1.00 | 21.52 |
| ATOM | 2775 | O | HOH | W | 154 | 19.181 | −5.396 | 13.023 | 1.00 | 33.83 |
| ATOM | 2776 | O | HOH | W | 155 | −3.698 | −11.419 | 33.622 | 1.00 | 22.32 |
| ATOM | 2777 | O | HOH | W | 156 | 24.773 | −7.687 | 26.828 | 1.00 | 25.23 |
| ATOM | 2778 | O | HOH | W | 157 | 11.338 | 6.981 | 10.450 | 1.00 | 25.98 |
| ATOM | 2779 | O | HOH | W | 158 | 7.187 | 4.097 | 9.722 | 1.00 | 25.73 |
| ATOM | 2780 | O | HOH | W | 159 | 10.571 | 18.276 | 18.940 | 1.00 | 26.56 |
| ATOM | 2781 | O | HOH | W | 160 | 29.444 | 8.138 | 18.655 | 1.00 | 25.10 |
| ATOM | 2782 | O | HOH | W | 161 | −4.733 | −16.782 | 12.321 | 1.00 | 23.51 |
| ATOM | 2783 | O | HOH | W | 162 | 24.194 | 7.218 | 40.602 | 1.00 | 28.88 |
| ATOM | 2784 | O | HOH | W | 163 | 13.925 | −7.040 | −5.961 | 1.00 | 29.58 |
| ATOM | 2785 | O | HOH | W | 164 | −8.456 | 17.425 | 10.469 | 1.00 | 25.03 |
| ATOM | 2786 | O | HOH | W | 165 | 20.283 | 7.809 | 6.109 | 1.00 | 25.58 |
| ATOM | 2787 | O | HOH | W | 166 | 11.226 | 1.360 | 14.389 | 1.00 | 28.28 |
| ATOM | 2788 | O | HOH | W | 167 | 17.600 | 11.985 | 25.766 | 1.00 | 28.21 |
| ATOM | 2789 | O | HOH | W | 168 | −1.369 | 0.364 | 6.150 | 1.00 | 26.61 |
| ATOM | 2790 | O | HOH | W | 169 | 1.250 | 6.912 | 6.112 | 1.00 | 22.96 |
| ATOM | 2791 | O | HOH | W | 170 | 13.982 | 16.820 | 19.827 | 1.00 | 26.70 |
| ATOM | 2792 | O | HOH | W | 171 | 8.401 | 6.117 | 8.285 | 1.00 | 29.37 |
| ATOM | 2793 | O | HOH | W | 172 | 1.561 | 32.980 | 20.315 | 1.00 | 25.71 |
| ATOM | 2794 | O | HOH | W | 173 | 8.687 | −4.960 | 0.724 | 1.00 | 20.63 |
| ATOM | 2795 | O | HOH | W | 174 | 16.762 | −8.584 | 32.541 | 1.00 | 22.53 |
| ATOM | 2796 | O | HOH | W | 175 | −8.928 | 23.790 | 25.122 | 1.00 | 26.68 |
| ATOM | 2797 | O | HOH | W | 176 | 30.774 | −10.692 | 12.781 | 1.00 | 29.33 |
| ATOM | 2798 | O | HOH | W | 177 | −4.491 | −0.583 | 48.435 | 1.00 | 27.59 |
| ATOM | 2799 | O | HOH | W | 178 | −10.254 | 8.887 | 25.677 | 1.00 | 27.41 |
| ATOM | 2800 | O | HOH | W | 179 | 12.487 | −14.347 | 0.366 | 1.00 | 29.02 |
| ATOM | 2801 | O | HOH | W | 180 | −2.194 | −3.193 | 5.572 | 1.00 | 37.26 |
| ATOM | 2802 | O | HOH | W | 181 | 21.240 | −14.747 | 4.723 | 1.00 | 24.12 |
| ATOM | 2803 | O | HOH | W | 182 | 25.238 | 15.872 | 21.083 | 1.00 | 33.15 |
| ATOM | 2804 | O | HOH | W | 183 | 27.650 | 8.541 | 12.497 | 1.00 | 23.15 |
| ATOM | 2805 | O | HOH | W | 184 | 35.303 | 3.837 | 26.688 | 1.00 | 23.28 |
| ATOM | 2806 | O | HOH | W | 185 | −3.730 | 14.555 | 34.107 | 1.00 | 23.34 |
| ATOM | 2807 | O | HOH | W | 186 | −8.927 | −4.660 | 10.381 | 1.00 | 28.70 |
| ATOM | 2808 | O | HOH | W | 187 | 40.695 | −1.179 | 15.166 | 1.00 | 31.35 |
| ATOM | 2809 | O | HOH | W | 188 | −0.356 | −22.615 | 31.607 | 1.00 | 35.28 |
| ATOM | 2810 | O | HOH | W | 189 | 3.463 | 34.674 | 22.382 | 1.00 | 27.20 |
| ATOM | 2811 | O | HOH | W | 190 | 8.911 | 2.196 | 11.376 | 1.00 | 19.30 |
| ATOM | 2812 | O | HOH | W | 191 | −2.598 | −24.115 | 28.861 | 1.00 | 26.04 |
| ATOM | 2813 | O | HOH | W | 192 | 8.418 | −15.510 | 34.179 | 1.00 | 38.02 |
| ATOM | 2814 | O | HOH | W | 193 | 15.703 | −17.633 | −0.060 | 1.00 | 35.88 |
| ATOM | 2815 | O | HOH | W | 194 | 32.518 | 0.997 | 3.614 | 1.00 | 34.25 |
| ATOM | 2816 | O | HOH | W | 195 | 19.062 | −16.798 | 10.198 | 1.00 | 27.69 |
| ATOM | 2817 | O | HOH | W | 196 | 13.216 | 8.976 | 10.392 | 1.00 | 27.61 |
| ATOM | 2818 | O | HOH | W | 197 | 2.647 | −0.086 | 12.019 | 1.00 | 23.91 |
| ATOM | 2819 | O | HOH | W | 198 | −1.276 | −7.561 | 35.325 | 1.00 | 25.96 |
| ATOM | 2820 | O | HOH | W | 199 | 4.267 | −22.362 | 30.170 | 1.00 | 35.68 |
| ATOM | 2821 | O | HOH | W | 200 | 21.787 | 10.595 | 30.558 | 1.00 | 32.19 |
| ATOM | 2822 | O | HOH | W | 201 | 21.638 | 20.389 | 14.210 | 1.00 | 43.09 |
| ATOM | 2823 | O | HOH | W | 202 | 5.373 | −10.297 | 38.927 | 1.00 | 36.29 |
| ATOM | 2824 | O | HOH | W | 203 | 29.035 | −9.588 | 1.139 | 1.00 | 25.61 |
| ATOM | 2825 | O | HOH | W | 204 | 0.403 | −17.629 | 11.462 | 1.00 | 25.52 |
| ATOM | 2826 | O | HOH | W | 205 | 15.896 | −2.824 | 23.429 | 1.00 | 9.81 |
| ATOM | 2827 | O | HOH | W | 206 | 13.783 | −1.283 | 21.923 | 1.00 | 12.03 |
| ATOM | 2828 | O | HOH | W | 207 | 16.854 | −0.738 | 21.328 | 1.00 | 11.76 |
| ATOM | 2829 | O | HOH | W | 208 | 14.946 | −0.465 | 19.263 | 1.00 | 10.55 |
| ATOM | 2830 | O | HOH | W | 209 | −5.566 | −9.500 | 29.835 | 1.00 | 34.12 |
| ATOM | 2831 | O | HOH | W | 210 | 11.435 | 14.251 | 33.693 | 1.00 | 48.96 |
| ATOM | 2832 | O | HOH | W | 211 | 3.944 | 31.281 | 18.816 | 1.00 | 43.83 |
| ATOM | 2833 | O | HOH | W | 212 | 21.511 | 8.201 | 40.401 | 1.00 | 42.14 |
| ATOM | 2834 | O | HOH | W | 213 | 44.825 | 1.178 | 19.793 | 1.00 | 52.54 |
| ATOM | 2835 | O | HOH | W | 214 | 9.397 | 20.244 | 23.958 | 1.00 | 30.91 |
| ATOM | 2836 | O | HOH | W | 215 | 18.636 | −1.182 | 15.865 | 1.00 | 23.82 |
| ATOM | 2837 | O | HOH | W | 216 | 18.955 | −2.693 | 17.957 | 1.00 | 28.82 |
| ATOM | 2838 | O | HOH | W | 217 | 26.248 | −9.541 | 27.965 | 1.00 | 25.10 |
| ATOM | 2839 | O | HOH | W | 218 | 24.703 | −9.867 | 30.146 | 1.00 | 25.52 |
| ATOM | 2840 | O | HOH | W | 219 | 22.600 | −9.260 | 25.832 | 1.00 | 26.71 |
| ATOM | 2841 | O | HOH | W | 220 | 1.825 | 10.054 | 39.457 | 1.00 | 25.12 |
| ATOM | 2842 | O | HOH | W | 221 | 3.585 | 4.590 | 45.058 | 1.00 | 28.59 |
| ATOM | 2843 | O | HOH | W | 222 | 3.777 | −6.424 | 30.447 | 1.00 | 24.42 |
| TER | 2844 | | HOH | W | 222 | | | | | |
| ATOM | 2845 | ZN | ZN | B | 864 | 13.210 | 0.069 | 20.406 | 1.00 | 15.15 |
| ATOM | 2846 | MG | MG | B | 865 | 15.487 | −0.725 | 23.006 | 1.00 | 8.84 |
| TER | 2847 | | MG | B | 865 | | | | | |
| END | | | | | | | | | | |

TABLE 5
Atomic coordinates for PDE5* complex with UK-088, 800
Atom Number, Atom Type, Residue Type, Residue Number Cartesian Coordinates
X, Y, Z, Atom Occupancy (O), Temperature Factor (B) and Atom Type

| | | | | | | X | Y | Z | O | B |
|---|---|---|---|---|---|---|---|---|---|---|
| ATOM | 1 | CB | GLU | A | 536 | 9.887 | 25.182 | 24.787 | 1.00 | 33.98 |
| ATOM | 2 | CG | GLU | A | 536 | 11.305 | 25.697 | 24.635 | 1.00 | 42.32 |
| ATOM | 3 | CD | GLU | A | 536 | 11.492 | 27.074 | 25.245 | 1.00 | 47.12 |
| ATOM | 4 | OE1 | GLU | A | 536 | 11.290 | 27.223 | 26.470 | 1.00 | 54.45 |
| ATOM | 5 | OE2 | GLU | A | 536 | 11.842 | 28.012 | 24.499 | 1.00 | 51.15 |
| ATOM | 6 | C | GLU | A | 536 | 7.942 | 25.044 | 26.327 | 1.00 | 37.22 |
| ATOM | 7 | O | GLU | A | 536 | 7.222 | 24.272 | 25.690 | 1.00 | 36.76 |
| ATOM | 8 | N | GLU | A | 536 | 9.991 | 23.667 | 26.752 | 1.00 | 36.93 |
| ATOM | 9 | CA | GLU | A | 536 | 9.456 | 24.961 | 26.239 | 1.00 | 37.47 |
| ATOM | 10 | N | THR | A | 537 | 7.463 | 25.990 | 27.125 | 1.00 | 37.34 |
| ATOM | 11 | CA | THR | A | 537 | 6.035 | 26.189 | 27.289 | 1.00 | 36.78 |
| ATOM | 12 | CB | THR | A | 537 | 5.750 | 27.235 | 28.387 | 1.00 | 43.61 |
| ATOM | 13 | OG1 | THR | A | 537 | 6.425 | 28.457 | 28.069 | 1.00 | 45.23 |
| ATOM | 14 | CG2 | THR | A | 537 | 6.241 | 26.731 | 29.739 | 1.00 | 34.90 |
| ATOM | 15 | C | THR | A | 537 | 5.452 | 26.660 | 25.957 | 1.00 | 35.15 |
| ATOM | 16 | O | THR | A | 537 | 4.239 | 26.661 | 25.764 | 1.00 | 34.30 |
| ATOM | 17 | N | ARG | A | 538 | 6.336 | 27.046 | 25.040 | 1.00 | 32.72 |
| ATOM | 18 | CA | ARG | A | 538 | 5.943 | 27.514 | 23.712 | 1.00 | 31.35 |
| ATOM | 19 | CB | ARG | A | 538 | 7.143 | 28.133 | 23.002 | 1.00 | 33.78 |
| ATOM | 20 | CG | ARG | A | 538 | 6.910 | 28.452 | 21.531 | 1.00 | 42.77 |
| ATOM | 21 | CD | ARG | A | 538 | 6.041 | 29.690 | 21.340 | 1.00 | 51.85 |
| ATOM | 22 | NE | ARG | A | 538 | 4.638 | 29.475 | 21.687 | 1.00 | 50.21 |
| ATOM | 23 | CZ | ARG | A | 538 | 3.715 | 30.433 | 21.672 | 1.00 | 49.57 |
| ATOM | 24 | NH1 | ARG | A | 538 | 4.047 | 31.673 | 21.330 | 1.00 | 48.08 |
| ATOM | 25 | NH2 | ARG | A | 538 | 2.458 | 30.154 | 21.992 | 1.00 | 49.14 |
| ATOM | 26 | C | ARG | A | 538 | 5.405 | 26.369 | 22.861 | 1.00 | 30.33 |
| ATOM | 27 | O | ARG | A | 538 | 4.381 | 26.501 | 22.187 | 1.00 | 28.72 |
| ATOM | 28 | N | GLU | A | 539 | 6.120 | 25.250 | 22.876 | 1.00 | 28.94 |
| ATOM | 29 | CA | GLU | A | 539 | 5.709 | 24.080 | 22.115 | 1.00 | 25.47 |
| ATOM | 30 | CB | GLU | A | 539 | 6.807 | 23.024 | 22.141 | 1.00 | 30.35 |
| ATOM | 31 | CG | GLU | A | 539 | 8.055 | 23.426 | 21.391 | 1.00 | 31.75 |
| ATOM | 32 | CD | GLU | A | 539 | 9.188 | 22.463 | 21.628 | 1.00 | 31.48 |
| ATOM | 33 | OE1 | GLU | A | 539 | 9.689 | 22.410 | 22.771 | 1.00 | 46.55 |
| ATOM | 34 | OE2 | GLU | A | 539 | 9.575 | 21.754 | 20.676 | 1.00 | 57.06 |
| ATOM | 35 | C | GLU | A | 539 | 4.437 | 23.511 | 22.718 | 1.00 | 23.49 |
| ATOM | 36 | O | GLU | A | 539 | 3.599 | 22.952 | 22.011 | 1.00 | 21.01 |
| ATOM | 37 | N | LEU | A | 540 | 4.299 | 23.653 | 24.032 | 1.00 | 22.37 |
| ATOM | 38 | CA | LEU | A | 540 | 3.114 | 23.159 | 24.713 | 1.00 | 22.10 |
| ATOM | 39 | CB | LEU | A | 540 | 3.302 | 23.223 | 26.231 | 1.00 | 19.44 |
| ATOM | 40 | CG | LEU | A | 540 | 2.098 | 22.763 | 27.059 | 1.00 | 22.04 |
| ATOM | 41 | CD1 | LEU | A | 540 | 1.686 | 21.365 | 26.627 | 1.00 | 19.81 |
| ATOM | 42 | CD2 | LEU | A | 540 | 2.441 | 22.783 | 28.539 | 1.00 | 38.12 |
| ATOM | 43 | C | LEU | A | 540 | 1.907 | 23.995 | 24.300 | 1.00 | 21.23 |
| ATOM | 44 | O | LEU | A | 540 | 0.844 | 23.461 | 23.990 | 1.00 | 19.37 |
| ATOM | 45 | N | GLN | A | 541 | 2.074 | 25.313 | 24.283 | 1.00 | 20.36 |
| ATOM | 46 | CA | GLN | A | 541 | 0.972 | 26.185 | 23.905 | 1.00 | 18.42 |
| ATOM | 47 | CB | GLN | A | 541 | 1.410 | 27.651 | 23.966 | 1.00 | 20.67 |
| ATOM | 48 | CG | GLN | A | 541 | 1.873 | 28.081 | 25.350 | 1.00 | 24.12 |
| ATOM | 49 | CD | GLN | A | 541 | 2.356 | 29.519 | 25.401 | 1.00 | 24.37 |
| ATOM | 50 | OE1 | GLN | A | 541 | 3.047 | 29.986 | 24.497 | 1.00 | 17.86 |
| ATOM | 51 | NE2 | GLN | A | 541 | 2.008 | 30.223 | 26.474 | 1.00 | 20.12 |
| ATOM | 52 | C | GLN | A | 541 | 0.477 | 25.833 | 22.505 | 1.00 | 17.74 |
| ATOM | 53 | O | GLN | A | 541 | −0.726 | 25.799 | 22.254 | 1.00 | 19.08 |
| ATOM | 54 | N | SER | A | 542 | 1.404 | 25.552 | 21.596 | 1.00 | 15.82 |
| ATOM | 55 | CA | SER | A | 542 | 1.026 | 25.207 | 20.231 | 1.00 | 18.42 |
| ATOM | 56 | CB | SER | A | 542 | 2.263 | 25.179 | 19.329 | 1.00 | 20.91 |
| ATOM | 57 | OG | SER | A | 542 | 2.857 | 26.463 | 19.251 | 1.00 | 37.22 |
| ATOM | 58 | C | SER | A | 542 | 0.320 | 23.858 | 20.174 | 1.00 | 16.82 |
| ATOM | 59 | O | SER | A | 542 | −0.710 | 23.709 | 19.520 | 1.00 | 17.71 |
| ATOM | 60 | N | LEU | A | 543 | 0.875 | 22.874 | 20.872 | 1.00 | 17.64 |
| ATOM | 61 | CA | LEU | A | 543 | 0.297 | 21.538 | 20.878 | 1.00 | 17.88 |
| ATOM | 62 | CB | LEU | A | 543 | 1.269 | 20.550 | 21.531 | 1.00 | 13.54 |
| ATOM | 63 | CG | LEU | A | 543 | 0.762 | 19.113 | 21.695 | 1.00 | 17.02 |
| ATOM | 64 | CD1 | LEU | A | 543 | 0.501 | 18.504 | 20.329 | 1.00 | 14.15 |
| ATOM | 65 | CD2 | LEU | A | 543 | 1.789 | 18.284 | 22.465 | 1.00 | 10.59 |
| ATOM | 66 | C | LEU | A | 543 | −1.049 | 21.468 | 21.592 | 1.00 | 18.33 |
| ATOM | 67 | O | LEU | A | 543 | −2.013 | 20.914 | 21.064 | 1.00 | 19.18 |
| ATOM | 68 | N | ALA | A | 544 | −1.112 | 22.038 | 22.790 | 1.00 | 18.60 |
| ATOM | 69 | CA | ALA | A | 544 | −2.332 | 22.003 | 23.588 | 1.00 | 19.09 |
| ATOM | 70 | CB | ALA | A | 544 | −2.053 | 22.573 | 24.972 | 1.00 | 17.83 |
| ATOM | 71 | C | ALA | A | 544 | −3.527 | 22.717 | 22.967 | 1.00 | 21.56 |
| ATOM | 72 | O | ALA | A | 544 | −4.667 | 22.306 | 23.165 | 1.00 | 23.51 |
| ATOM | 73 | N | ALA | A | 545 | −3.269 | 23.782 | 22.217 | 1.00 | 23.98 |
| ATOM | 74 | CA | ALA | A | 545 | −4.348 | 24.547 | 21.599 | 1.00 | 24.34 |
| ATOM | 75 | CB | ALA | A | 545 | −3.861 | 25.952 | 21.274 | 1.00 | 26.53 |
| ATOM | 76 | C | ALA | A | 545 | −4.901 | 23.891 | 20.341 | 1.00 | 25.76 |
| ATOM | 77 | O | ALA | A | 545 | −6.065 | 24.079 | 19.992 | 1.00 | 28.16 |
| ATOM | 78 | N | ALA | A | 546 | −4.059 | 23.115 | 19.668 | 1.00 | 24.74 |
| ATOM | 79 | CA | ALA | A | 546 | −4.441 | 22.443 | 18.430 | 1.00 | 24.22 |
| ATOM | 80 | CB | ALA | A | 546 | −3.239 | 21.701 | 17.861 | 1.00 | 22.00 |
| ATOM | 81 | C | ALA | A | 546 | −5.621 | 21.485 | 18.529 | 1.00 | 21.89 |
| ATOM | 82 | O | ALA | A | 546 | −5.803 | 20.791 | 19.529 | 1.00 | 23.27 |
| ATOM | 83 | N | VAL | A | 547 | −6.433 | 21.464 | 17.478 | 1.00 | 21.72 |
| ATOM | 84 | CA | VAL | A | 547 | −7.560 | 20.548 | 17.418 | 1.00 | 21.65 |
| ATOM | 85 | CB | VAL | A | 547 | −8.660 | 21.045 | 16.454 | 1.00 | 23.33 |
| ATOM | 86 | CG1 | VAL | A | 547 | −9.754 | 19.993 | 16.326 | 1.00 | 25.11 |
| ATOM | 87 | CG2 | VAL | A | 547 | −9.250 | 22.355 | 16.965 | 1.00 | 23.58 |
| ATOM | 88 | C | VAL | A | 547 | −6.925 | 19.284 | 16.850 | 1.00 | 22.28 |
| ATOM | 89 | O | VAL | A | 547 | −6.280 | 19.329 | 15.805 | 1.00 | 24.96 |
| ATOM | 90 | N | VAL | A | 548 | −7.090 | 18.165 | 17.542 | 1.00 | 21.82 |
| ATOM | 91 | CA | VAL | A | 548 | −6.498 | 16.909 | 17.098 | 1.00 | 19.25 |
| ATOM | 92 | CB | VAL | A | 548 | −6.387 | 15.925 | 18.278 | 1.00 | 19.56 |
| ATOM | 93 | CG1 | VAL | A | 548 | −5.669 | 14.659 | 17.840 | 1.00 | 23.78 |
| ATOM | 94 | CG2 | VAL | A | 548 | −5.656 | 16.593 | 19.438 | 1.00 | 24.95 |
| ATOM | 95 | C | VAL | A | 548 | −7.284 | 16.241 | 15.975 | 1.00 | 17.26 |
| ATOM | 96 | O | VAL | A | 548 | −8.453 | 15.904 | 16.146 | 1.00 | 19.24 |
| ATOM | 97 | N | PRO | A | 549 | −6.646 | 16.038 | 14.808 | 1.00 | 17.33 |
| ATOM | 98 | CD | PRO | A | 549 | −5.293 | 16.478 | 14.431 | 1.00 | 18.64 |
| ATOM | 99 | CA | PRO | A | 549 | −7.315 | 15.401 | 13.670 | 1.00 | 19.43 |
| ATOM | 100 | CB | PRO | A | 549 | −6.245 | 15.425 | 12.575 | 1.00 | 17.30 |
| ATOM | 101 | CG | PRO | A | 549 | −5.420 | 16.615 | 12.931 | 1.00 | 23.24 |
| ATOM | 102 | C | PRO | A | 549 | −7.742 | 13.982 | 14.023 | 1.00 | 20.23 |
| ATOM | 103 | O | PRO | A | 549 | −7.259 | 13.402 | 15.001 | 1.00 | 16.82 |
| ATOM | 104 | N | SER | A | 550 | −8.645 | 13.431 | 13.220 | 1.00 | 17.57 |
| ATOM | 105 | CA | SER | A | 550 | −9.153 | 12.081 | 13.432 | 1.00 | 20.68 |
| ATOM | 106 | CB | SER | A | 550 | −10.343 | 11.822 | 12.512 | 1.00 | 17.19 |
| ATOM | 107 | OG | SER | A | 550 | −9.934 | 11.861 | 11.153 | 1.00 | 18.60 |
| ATOM | 108 | C | SER | A | 550 | −8.075 | 11.055 | 13.127 | 1.00 | 17.34 |
| ATOM | 109 | O | SER | A | 550 | −7.096 | 11.357 | 12.443 | 1.00 | 16.71 |
| ATOM | 110 | N | ALA | A | 551 | −8.257 | 9.841 | 13.637 | 1.00 | 18.46 |
| ATOM | 111 | CA | ALA | A | 551 | −7.304 | 8.769 | 13.380 | 1.00 | 19.34 |
| ATOM | 112 | CB | ALA | A | 551 | −7.706 | 7.507 | 14.143 | 1.00 | 17.06 |
| ATOM | 113 | C | ALA | A | 551 | −7.322 | 8.502 | 11.879 | 1.00 | 18.62 |
| ATOM | 114 | O | ALA | A | 551 | −6.284 | 8.262 | 11.261 | 1.00 | 20.73 |
| ATOM | 115 | N | GLN | A | 552 | −8.518 | 8.554 | 11.300 | 1.00 | 19.78 |
| ATOM | 116 | CA | GLN | A | 552 | −8.700 | 8.327 | 9.869 | 1.00 | 21.36 |
| ATOM | 117 | CB | GLN | A | 552 | −10.175 | 8.528 | 9.502 | 1.00 | 23.40 |
| ATOM | 118 | CG | GLN | A | 552 | −10.541 | 8.181 | 8.067 | 1.00 | 31.79 |
| ATOM | 119 | CD | GLN | A | 552 | −12.026 | 8.358 | 7.797 | 1.00 | 35.80 |
| ATOM | 120 | OE1 | GLN | A | 552 | −12.864 | 7.675 | 8.390 | 1.00 | 46.36 |
| ATOM | 121 | NE2 | GLN | A | 552 | −12.358 | 9.283 | 6.902 | 1.00 | 49.03 |
| ATOM | 122 | C | GLN | A | 552 | −7.820 | 9.301 | 9.086 | 1.00 | 21.35 |
| ATOM | 123 | O | GLN | A | 552 | −7.040 | 8.897 | 8.219 | 1.00 | 21.48 |
| ATOM | 124 | N | THR | A | 553 | −7.938 | 10.584 | 9.408 | 1.00 | 19.75 |
| ATOM | 125 | CA | THR | A | 553 | −7.151 | 11.612 | 8.739 | 1.00 | 19.52 |
| ATOM | 126 | CB | THR | A | 553 | −7.501 | 13.017 | 9.268 | 1.00 | 18.18 |
| ATOM | 127 | OG1 | THR | A | 553 | −8.871 | 13.318 | 8.971 | 1.00 | 19.96 |
| ATOM | 128 | CG2 | THR | A | 553 | −6.597 | 14.066 | 8.627 | 1.00 | 18.92 |
| ATOM | 129 | C | THR | A | 553 | −5.654 | 11.396 | 8.926 | 1.00 | 20.00 |
| ATOM | 130 | O | THR | A | 553 | −4.875 | 11.523 | 7.980 | 1.00 | 19.53 |
| ATOM | 131 | N | LEU | A | 554 | −5.260 | 11.062 | 10.150 | 1.00 | 19.10 |
| ATOM | 132 | CA | LEU | A | 554 | −3.855 | 10.853 | 10.474 | 1.00 | 20.03 |
| ATOM | 133 | CB | LEU | A | 554 | −3.650 | 10.974 | 11.987 | 1.00 | 17.91 |
| ATOM | 134 | CG | LEU | A | 554 | −4.091 | 12.323 | 12.571 | 1.00 | 18.40 |
| ATOM | 135 | CD1 | LEU | A | 554 | −3.972 | 12.316 | 14.082 | 1.00 | 16.11 |
| ATOM | 136 | CD2 | LEU | A | 554 | −3.237 | 13.436 | 11.977 | 1.00 | 23.68 |
| ATOM | 137 | C | LEU | A | 554 | −3.303 | 9.521 | 9.970 | 1.00 | 19.88 |
| ATOM | 138 | O | LEU | A | 554 | −2.093 | 9.304 | 9.994 | 1.00 | 19.74 |
| ATOM | 139 | N | LYS | A | 555 | −4.198 | 8.637 | 9.533 | 1.00 | 17.37 |
| ATOM | 140 | CA | LYS | A | 555 | −3.833 | 7.327 | 8.997 | 1.00 | 19.69 |
| ATOM | 141 | CB | LYS | A | 555 | −3.032 | 7.523 | 7.708 | 1.00 | 26.45 |
| ATOM | 142 | CG | LYS | A | 555 | −3.740 | 8.429 | 6.708 | 1.00 | 33.54 |
| ATOM | 143 | CD | LYS | A | 555 | −2.829 | 8.837 | 5.565 | 1.00 | 36.95 |
| ATOM | 144 | CE | LYS | A | 555 | −3.542 | 9.803 | 4.633 | 1.00 | 36.53 |
| ATOM | 145 | NZ | LYS | A | 555 | −2.645 | 10.308 | 3.562 | 1.00 | 43.43 |
| ATOM | 146 | C | LYS | A | 555 | −3.043 | 6.454 | 9.976 | 1.00 | 19.45 |
| ATOM | 147 | O | LYS | A | 555 | −2.294 | 5.569 | 9.565 | 1.00 | 18.29 |
| ATOM | 148 | N | ILE | A | 556 | −3.227 | 6.688 | 11.271 | 1.00 | 17.06 |
| ATOM | 149 | CA | ILE | A | 556 | −2.501 | 5.917 | 12.273 | 1.00 | 16.83 |
| ATOM | 150 | CB | ILE | A | 556 | −2.475 | 6.638 | 13.641 | 1.00 | 13.95 |
| ATOM | 151 | CG2 | ILE | A | 556 | −1.565 | 7.856 | 13.569 | 1.00 | 16.87 |
| ATOM | 152 | CG1 | ILE | A | 556 | −3.897 | 7.017 | 14.062 | 1.00 | 14.93 |
| ATOM | 153 | CD1 | ILE | A | 556 | −3.981 | 7.598 | 15.460 | 1.00 | 19.73 |
| ATOM | 154 | C | ILE | A | 556 | −3.019 | 4.496 | 12.489 | 1.00 | 14.60 |
| ATOM | 155 | O | ILE | A | 556 | −2.443 | 3.745 | 13.271 | 1.00 | 12.74 |
| ATOM | 156 | N | THR | A | 557 | −4.099 | 4.125 | 11.806 | 1.00 | 14.27 |
| ATOM | 157 | CA | THR | A | 557 | −4.653 | 2.776 | 11.938 | 1.00 | 15.05 |
| ATOM | 158 | CB | THR | A | 557 | −6.187 | 2.772 | 11.699 | 1.00 | 21.22 |
| ATOM | 159 | OG1 | THR | A | 557 | −6.813 | 3.726 | 12.567 | 1.00 | 20.77 |
| ATOM | 160 | CG2 | THR | A | 557 | −6.773 | 1.398 | 11.982 | 1.00 | 21.26 |
| ATOM | 161 | C | THR | A | 557 | −3.991 | 1.855 | 10.907 | 1.00 | 14.39 |
| ATOM | 162 | O | THR | A | 557 | −4.131 | 0.631 | 10.955 | 1.00 | 16.89 |
| ATOM | 163 | N | ASP | A | 558 | −3.257 | 2.456 | 9.979 | 1.00 | 14.56 |
| ATOM | 164 | CA | ASP | A | 558 | −2.593 | 1.702 | 8.923 | 1.00 | 14.77 |
| ATOM | 165 | CB | ASP | A | 558 | −2.374 | 2.613 | 7.713 | 1.00 | 20.77 |
| ATOM | 166 | CG | ASP | A | 558 | −3.648 | 3.312 | 7.274 | 1.00 | 30.09 |
| ATOM | 167 | OD1 | ASP | A | 558 | −4.657 | 2.616 | 7.031 | 1.00 | 38.75 |
| ATOM | 168 | OD2 | ASP | A | 558 | −3.642 | 4.555 | 7.173 | 1.00 | 46.69 |
| ATOM | 169 | C | ASP | A | 558 | −1.258 | 1.086 | 9.351 | 1.00 | 14.68 |
| ATOM | 170 | O | ASP | A | 558 | −0.365 | 1.787 | 9.829 | 1.00 | 13.87 |
| ATOM | 171 | N | PHE | A | 559 | −1.124 | −0.227 | 9.175 | 1.00 | 13.82 |
| ATOM | 172 | CA | PHE | A | 559 | 0.119 | −0.916 | 9.526 | 1.00 | 13.07 |
| ATOM | 173 | CB | PHE | A | 559 | 0.000 | −2.427 | 9.283 | 1.00 | 14.41 |
| ATOM | 174 | CG | PHE | A | 559 | −0.732 | −3.175 | 10.366 | 1.00 | 18.21 |
| ATOM | 175 | CD1 | PHE | A | 559 | −0.289 | −3.134 | 11.680 | 1.00 | 14.30 |
| ATOM | 176 | CD2 | PHE | A | 559 | −1.836 | −3.960 | 10.060 | 1.00 | 16.61 |
| ATOM | 177 | CE1 | PHE | A | 559 | −0.931 | −3.866 | 12.668 | 1.00 | 15.41 |
| ATOM | 178 | CE2 | PHE | A | 559 | −2.482 | −4.692 | 11.043 | 1.00 | 15.41 |
| ATOM | 179 | CZ | PHE | A | 559 | −2.031 | −4.647 | 12.344 | 1.00 | 12.10 |
| ATOM | 180 | C | PHE | A | 559 | 1.277 | −0.386 | 8.680 | 1.00 | 11.71 |
| ATOM | 181 | O | PHE | A | 559 | 2.431 | −0.411 | 9.109 | 1.00 | 12.25 |
| ATOM | 182 | N | SER | A | 560 | 0.965 | 0.079 | 7.473 | 1.00 | 13.82 |
| ATOM | 183 | CA | SER | A | 560 | 1.979 | 0.597 | 6.555 | 1.00 | 13.86 |
| ATOM | 184 | CB | SER | A | 560 | 1.538 | 0.384 | 5.106 | 1.00 | 19.52 |
| ATOM | 185 | OG | SER | A | 560 | 1.411 | −0.996 | 4.816 | 1.00 | 19.35 |
| ATOM | 186 | C | SER | A | 560 | 2.294 | 2.071 | 6.770 | 1.00 | 12.88 |
| ATOM | 187 | O | SER | A | 560 | 2.959 | 2.698 | 5.945 | 1.00 | 14.29 |
| ATOM | 188 | N | PHE | A | 561 | 1.815 | 2.608 | 7.886 | 1.00 | 12.82 |
| ATOM | 189 | CA | PHE | A | 561 | 2.021 | 4.009 | 8.261 | 1.00 | 13.63 |
| ATOM | 190 | CB | PHE | A | 561 | 1.567 | 4.207 | 9.713 | 1.00 | 14.59 |
| ATOM | 191 | CG | PHE | A | 561 | 1.835 | 5.583 | 10.258 | 1.00 | 12.08 |
| ATOM | 192 | CD1 | PHE | A | 561 | 0.956 | 6.625 | 10.014 | 1.00 | 17.74 |
| ATOM | 193 | CD2 | PHE | A | 561 | 2.967 | 5.829 | 11.022 | 1.00 | 10.31 |
| ATOM | 194 | CE1 | PHE | A | 561 | 1.199 | 7.891 | 10.524 | 1.00 | 21.75 |
| ATOM | 195 | CE2 | PHE | A | 561 | 3.220 | 7.093 | 11.537 | 1.00 | 13.27 |
| ATOM | 196 | CZ | PHE | A | 561 | 2.333 | 8.126 | 11.287 | 1.00 | 18.85 |
| ATOM | 197 | C | PHE | A | 561 | 3.466 | 4.498 | 8.125 | 1.00 | 14.38 |
| ATOM | 198 | O | PHE | A | 561 | 4.413 | 3.791 | 8.485 | 1.00 | 11.10 |
| ATOM | 199 | N | SER | A | 562 | 3.625 | 5.716 | 7.607 | 1.00 | 14.38 |
| ATOM | 200 | CA | SER | A | 562 | 4.939 | 6.334 | 7.453 | 1.00 | 13.72 |
| ATOM | 201 | CB | SER | A | 562 | 5.283 | 6.520 | 5.972 | 1.00 | 15.96 |
| ATOM | 202 | OG | SER | A | 562 | 6.542 | 7.159 | 5.835 | 1.00 | 23.25 |
| ATOM | 203 | C | SER | A | 562 | 4.940 | 7.695 | 8.159 | 1.00 | 12.53 |
| ATOM | 204 | O | SER | A | 562 | 4.009 | 8.490 | 8.014 | 1.00 | 12.55 |
| ATOM | 205 | N | ASP | A | 563 | 5.992 | 7.965 | 8.922 | 1.00 | 13.45 |
| ATOM | 206 | CA | ASP | A | 563 | 6.094 | 9.223 | 9.661 | 1.00 | 15.79 |
| ATOM | 207 | CB | ASP | A | 563 | 6.662 | 8.953 | 11.050 | 1.00 | 13.73 |
| ATOM | 208 | CG | ASP | A | 563 | 8.166 | 8.765 | 11.022 | 1.00 | 15.49 |
| ATOM | 209 | OD1 | ASP | A | 563 | 8.895 | 9.680 | 11.465 | 1.00 | 18.81 |
| ATOM | 210 | OD2 | ASP | A | 563 | 8.625 | 7.712 | 10.537 | 1.00 | 16.12 |
| ATOM | 211 | C | ASP | A | 563 | 7.009 | 10.232 | 8.974 | 1.00 | 16.63 |
| ATOM | 212 | O | ASP | A | 563 | 7.112 | 11.383 | 9.404 | 1.00 | 17.82 |
| ATOM | 213 | N | PHE | A | 564 | 7.673 | 9.796 | 7.911 | 1.00 | 16.51 |
| ATOM | 214 | CA | PHE | A | 564 | 8.633 | 10.635 | 7.205 | 1.00 | 18.23 |
| ATOM | 215 | CB | PHE | A | 564 | 9.207 | 9.871 | 6.011 | 1.00 | 15.87 |
| ATOM | 216 | CG | PHE | A | 564 | 10.446 | 10.494 | 5.438 | 1.00 | 26.61 |
| ATOM | 217 | CD1 | PHE | A | 564 | 11.599 | 10.592 | 6.201 | 1.00 | 20.64 |
| ATOM | 218 | CD2 | PHE | A | 564 | 10.458 | 10.991 | 4.144 | 1.00 | 27.32 |
| ATOM | 219 | CE1 | PHE | A | 564 | 12.742 | 11.174 | 5.685 | 1.00 | 30.38 |
| ATOM | 220 | CE2 | PHE | A | 564 | 11.599 | 11.574 | 3.622 | 1.00 | 33.45 |
| ATOM | 221 | CZ | PHE | A | 564 | 12.742 | 11.665 | 4.394 | 1.00 | 33.80 |
| ATOM | 222 | C | PHE | A | 564 | 8.157 | 12.010 | 6.740 | 1.00 | 18.52 |
| ATOM | 223 | O | PHE | A | 564 | 8.942 | 12.959 | 6.728 | 1.00 | 19.54 |
| ATOM | 224 | N | GLU | A | 565 | 6.885 | 12.122 | 6.369 | 1.00 | 16.14 |
| ATOM | 225 | CA | GLU | A | 565 | 6.340 | 13.391 | 5.890 | 1.00 | 19.03 |
| ATOM | 226 | CB | GLU | A | 565 | 5.277 | 13.142 | 4.813 | 1.00 | 18.54 |
| ATOM | 227 | CG | GLU | A | 565 | 5.708 | 12.238 | 3.668 | 1.00 | 33.46 |
| ATOM | 228 | CD | GLU | A | 565 | 6.004 | 10.818 | 4.117 | 1.00 | 40.92 |
| ATOM | 229 | OE1 | GLU | A | 565 | 5.205 | 10.258 | 4.899 | 1.00 | 41.02 |
| ATOM | 230 | OE2 | GLU | A | 565 | 7.032 | 10.259 | 3.680 | 1.00 | 50.71 |
| ATOM | 231 | C | GLU | A | 565 | 5.710 | 14.229 | 7.002 | 1.00 | 19.43 |
| ATOM | 232 | O | GLU | A | 565 | 5.158 | 15.301 | 6.740 | 1.00 | 20.33 |
| ATOM | 233 | N | LEU | A | 566 | 5.786 | 13.746 | 8.238 | 1.00 | 13.91 |
| ATOM | 234 | CA | LEU | A | 566 | 5.193 | 14.463 | 9.362 | 1.00 | 15.26 |
| ATOM | 235 | CB | LEU | A | 566 | 4.515 | 13.482 | 10.321 | 1.00 | 15.63 |
| ATOM | 236 | CG | LEU | A | 566 | 3.329 | 12.669 | 9.799 | 1.00 | 24.63 |
| ATOM | 237 | CD1 | LEU | A | 566 | 2.864 | 11.698 | 10.881 | 1.00 | 23.72 |
| ATOM | 238 | CD2 | LEU | A | 566 | 2.199 | 13.607 | 9.399 | 1.00 | 33.15 |
| ATOM | 239 | C | LEU | A | 566 | 6.187 | 15.306 | 10.147 | 1.00 | 14.43 |
| ATOM | 240 | O | LEU | A | 566 | 7.358 | 14.960 | 10.265 | 1.00 | 17.81 |
| ATOM | 241 | N | SER | A | 567 | 5.699 | 16.419 | 10.685 | 1.00 | 14.78 |
| ATOM | 242 | CA | SER | A | 567 | 6.517 | 17.317 | 11.486 | 1.00 | 14.98 |
| ATOM | 243 | CB | SER | A | 567 | 5.928 | 18.727 | 11.467 | 1.00 | 21.89 |
| ATOM | 244 | OG | SER | A | 567 | 4.679 | 18.752 | 12.144 | 1.00 | 17.63 |
| ATOM | 245 | C | SER | A | 567 | 6.492 | 16.797 | 12.922 | 1.00 | 13.89 |
| ATOM | 246 | O | SER | A | 567 | 5.654 | 15.963 | 13.268 | 1.00 | 13.82 |
| ATOM | 247 | N | ASP | A | 568 | 7.403 | 17.289 | 13.753 | 1.00 | 15.43 |
| ATOM | 248 | CA | ASP | A | 568 | 7.428 | 16.869 | 15.149 | 1.00 | 15.57 |
| ATOM | 249 | CB | ASP | A | 568 | 8.536 | 17.603 | 15.904 | 1.00 | 19.60 |
| ATOM | 250 | CG | ASP | A | 568 | 9.888 | 16.947 | 15.727 | 1.00 | 16.75 |
| ATOM | 251 | OD1 | ASP | A | 568 | 9.989 | 16.021 | 14.899 | 1.00 | 19.05 |
| ATOM | 252 | OD2 | ASP | A | 568 | 10.848 | 17.355 | 16.415 | 1.00 | 23.47 |
| ATOM | 253 | C | ASP | A | 568 | 6.076 | 17.145 | 15.805 | 1.00 | 16.52 |
| ATOM | 254 | O | ASP | A | 568 | 5.559 | 16.313 | 16.556 | 1.00 | 15.97 |
| ATOM | 255 | N | LEU | A | 569 | 5.505 | 18.311 | 15.516 | 1.00 | 15.09 |
| ATOM | 256 | CA | LEU | A | 569 | 4.213 | 18.683 | 16.076 | 1.00 | 15.08 |
| ATOM | 257 | CB | LEU | A | 569 | 3.815 | 20.093 | 15.620 | 1.00 | 16.05 |
| ATOM | 258 | CG | LEU | A | 569 | 2.385 | 20.516 | 15.974 | 1.00 | 23.47 |
| ATOM | 259 | CD1 | LEU | A | 569 | 2.203 | 20.527 | 17.489 | 1.00 | 21.65 |
| ATOM | 260 | CD2 | LEU | A | 569 | 2.103 | 21.896 | 15.398 | 1.00 | 27.54 |
| ATOM | 261 | C | LEU | A | 569 | 3.136 | 17.694 | 15.647 | 1.00 | 12.90 |
| ATOM | 262 | O | LEU | A | 569 | 2.279 | 17.308 | 16.441 | 1.00 | 16.26 |
| ATOM | 263 | N | GLU | A | 570 | 3.184 | 17.281 | 14.386 | 1.00 | 13.40 |
| ATOM | 264 | CA | GLU | A | 570 | 2.202 | 16.340 | 13.869 | 1.00 | 14.08 |
| ATOM | 265 | CB | GLU | A | 570 | 2.354 | 16.196 | 12.352 | 1.00 | 14.43 |
| ATOM | 266 | CG | GLU | A | 570 | 1.885 | 17.432 | 11.579 | 1.00 | 20.85 |
| ATOM | 267 | CD | GLU | A | 570 | 2.062 | 17.291 | 10.081 | 1.00 | 23.63 |
| ATOM | 268 | OE1 | GLU | A | 570 | 3.218 | 17.158 | 9.632 | 1.00 | 21.68 |
| ATOM | 269 | OE2 | GLU | A | 570 | 1.047 | 17.311 | 9.352 | 1.00 | 28.09 |
| ATOM | 270 | C | GLU | A | 570 | 2.309 | 14.978 | 14.547 | 1.00 | 14.43 |
| ATOM | 271 | O | GLU | A | 570 | 1.293 | 14.319 | 14.780 | 1.00 | 15.20 |
| ATOM | 272 | N | THR | A | 571 | 3.529 | 14.551 | 14.866 | 1.00 | 12.67 |
| ATOM | 273 | CA | THR | A | 571 | 3.694 | 13.259 | 15.538 | 1.00 | 12.83 |
| ATOM | 274 | CB | THR | A | 571 | 5.182 | 12.816 | 15.638 | 1.00 | 13.30 |
| ATOM | 275 | OG1 | THR | A | 571 | 5.915 | 13.737 | 16.452 | 1.00 | 12.68 |
| ATOM | 276 | CG2 | THR | A | 571 | 5.817 | 12.738 | 14.250 | 1.00 | 12.38 |
| ATOM | 277 | C | THR | A | 571 | 3.110 | 13.360 | 16.945 | 1.00 | 11.29 |
| ATOM | 278 | O | THR | A | 571 | 2.589 | 12.381 | 17.483 | 1.00 | 12.14 |
| ATOM | 279 | N | ALA | A | 572 | 3.202 | 14.548 | 17.538 | 1.00 | 12.42 |
| ATOM | 280 | CA | ALA | A | 572 | 2.659 | 14.780 | 18.869 | 1.00 | 11.84 |
| ATOM | 281 | CB | ALA | A | 572 | 3.090 | 16.142 | 19.388 | 1.00 | 12.18 |
| ATOM | 282 | C | ALA | A | 572 | 1.136 | 14.701 | 18.803 | 1.00 | 13.34 |
| ATOM | 283 | O | ALA | A | 572 | 0.500 | 14.128 | 19.683 | 1.00 | 11.72 |
| ATOM | 284 | N | LEU | A | 573 | 0.554 | 15.283 | 17.758 | 1.00 | 12.67 |
| ATOM | 285 | CA | LEU | A | 573 | −0.894 | 15.244 | 17.583 | 1.00 | 13.98 |
| ATOM | 286 | CB | LEU | A | 573 | −1.307 | 16.104 | 16.384 | 1.00 | 14.33 |
| ATOM | 287 | CG | LEU | A | 573 | −1.135 | 17.619 | 16.544 | 1.00 | 14.55 |
| ATOM | 288 | CD1 | LEU | A | 573 | −1.445 | 18.310 | 15.224 | 1.00 | 21.57 |
| ATOM | 289 | CD2 | LEU | A | 573 | −2.061 | 18.125 | 17.647 | 1.00 | 20.27 |
| ATOM | 290 | C | LEU | A | 573 | −1.338 | 13.795 | 17.366 | 1.00 | 15.26 |
| ATOM | 291 | O | LEU | A | 573 | −2.394 | 13.378 | 17.853 | 1.00 | 15.01 |
| ATOM | 292 | N | CYS | A | 574 | −0.534 | 13.030 | 16.632 | 1.00 | 13.44 |
| ATOM | 293 | CA | CYS | A | 574 | −0.852 | 11.625 | 16.385 | 1.00 | 13.06 |
| ATOM | 294 | CB | CYS | A | 574 | 0.187 | 10.974 | 15.470 | 1.00 | 12.27 |
| ATOM | 295 | SG | CYS | A | 574 | 0.049 | 11.382 | 13.714 | 1.00 | 15.24 |
| ATOM | 296 | C | CYS | A | 574 | −0.873 | 10.864 | 17.703 | 1.00 | 12.15 |
| ATOM | 297 | O | CYS | A | 574 | −1.710 | 9.991 | 17.919 | 1.00 | 13.51 |
| ATOM | 298 | N | THR | A | 575 | 0.066 | 11.194 | 18.581 | 1.00 | 11.60 |
| ATOM | 299 | CA | THR | A | 575 | 0.158 | 10.532 | 19.872 | 1.00 | 12.28 |
| ATOM | 300 | CB | THR | A | 575 | 1.453 | 10.949 | 20.592 | 1.00 | 10.90 |
| ATOM | 301 | OG1 | THR | A | 575 | 2.575 | 10.576 | 19.777 | 1.00 | 13.47 |
| ATOM | 302 | CG2 | THR | A | 575 | 1.565 | 10.264 | 21.944 | 1.00 | 10.06 |
| ATOM | 303 | C | THR | A | 575 | −1.068 | 10.841 | 20.726 | 1.00 | 11.74 |
| ATOM | 304 | O | THR | A | 575 | −1.613 | 9.952 | 21.379 | 1.00 | 10.78 |
| ATOM | 305 | N | ILE | A | 576 | −1.514 | 12.095 | 20.717 | 1.00 | 12.29 |
| ATOM | 306 | CA | ILE | A | 576 | −2.700 | 12.453 | 21.484 | 1.00 | 11.92 |
| ATOM | 307 | CB | ILE | A | 576 | −3.046 | 13.945 | 21.354 | 1.00 | 11.38 |
| ATOM | 308 | CG2 | ILE | A | 576 | −4.371 | 14.222 | 22.053 | 1.00 | 12.83 |
| ATOM | 309 | CG1 | ILE | A | 576 | −1.933 | 14.802 | 21.965 | 1.00 | 17.73 |
| ATOM | 310 | CD1 | ILE | A | 576 | −2.154 | 16.292 | 21.798 | 1.00 | 13.34 |
| ATOM | 311 | C | ILE | A | 576 | −3.880 | 11.636 | 20.960 | 1.00 | 12.34 |
| ATOM | 312 | O | ILE | A | 576 | −4.674 | 11.103 | 21.737 | 1.00 | 13.64 |
| ATOM | 313 | N | ARG | A | 577 | −3.985 | 11.532 | 19.638 | 1.00 | 13.14 |
| ATOM | 314 | CA | ARG | A | 577 | −5.071 | 10.772 | 19.032 | 1.00 | 13.22 |
| ATOM | 315 | CB | ARG | A | 577 | −5.029 | 10.888 | 17.503 | 1.00 | 12.21 |
| ATOM | 316 | CG | ARG | A | 577 | −6.113 | 10.084 | 16.795 | 1.00 | 12.42 |
| ATOM | 317 | CD | ARG | A | 577 | −7.492 | 10.383 | 17.365 | 1.00 | 10.79 |
| ATOM | 318 | NE | ARG | A | 577 | −7.912 | 11.757 | 17.111 | 1.00 | 18.46 |
| ATOM | 319 | CZ | ARG | A | 577 | −9.015 | 12.304 | 17.612 | 1.00 | 16.47 |
| ATOM | 320 | NH1 | ARG | A | 577 | −9.319 | 13.564 | 17.325 | 1.00 | 24.31 |
| ATOM | 321 | NH2 | ARG | A | 577 | −9.809 | 11.596 | 18.405 | 1.00 | 18.21 |
| ATOM | 322 | C | ARG | A | 577 | −5.029 | 9.304 | 19.455 | 1.00 | 12.45 |
| ATOM | 323 | O | ARG | A | 577 | −6.069 | 8.675 | 19.625 | 1.00 | 11.98 |
| ATOM | 324 | N | MET | A | 578 | −3.831 | 8.756 | 19.630 | 1.00 | 10.43 |
| ATOM | 325 | CA | MET | A | 578 | −3.716 | 7.367 | 20.058 | 1.00 | 11.28 |
| ATOM | 326 | CB | MET | A | 578 | −2.245 | 6.934 | 20.090 | 1.00 | 11.91 |
| ATOM | 327 | CG | MET | A | 578 | −1.605 | 6.808 | 18.716 | 1.00 | 12.93 |
| ATOM | 328 | SD | MET | A | 578 | 0.134 | 6.324 | 18.830 | 1.00 | 16.07 |
| ATOM | 329 | CE | MET | A | 578 | 0.781 | 7.021 | 17.300 | 1.00 | 19.76 |
| ATOM | 330 | C | MET | A | 578 | −4.332 | 7.191 | 21.445 | 1.00 | 12.18 |
| ATOM | 331 | O | MET | A | 578 | −5.103 | 6.260 | 21.675 | 1.00 | 11.26 |
| ATOM | 332 | N | PHE | A | 579 | −3.982 | 8.080 | 22.372 | 1.00 | 11.60 |
| ATOM | 333 | CA | PHE | A | 579 | −4.521 | 8.004 | 23.725 | 1.00 | 11.60 |
| ATOM | 334 | CB | PHE | A | 579 | −3.921 | 9.097 | 24.619 | 1.00 | 12.17 |
| ATOM | 335 | CG | PHE | A | 579 | −2.584 | 8.748 | 25.201 | 1.00 | 9.68 |
| ATOM | 336 | CD1 | PHE | A | 579 | −1.415 | 9.023 | 24.510 | 1.00 | 13.42 |
| ATOM | 337 | CD2 | PHE | A | 579 | −2.497 | 8.148 | 26.450 | 1.00 | 12.49 |
| ATOM | 338 | CE1 | PHE | A | 579 | −0.178 | 8.709 | 25.054 | 1.00 | 10.87 |
| ATOM | 339 | CE2 | PHE | A | 579 | −1.264 | 7.829 | 27.000 | 1.00 | 13.01 |
| ATOM | 340 | CZ | PHE | A | 579 | −0.103 | 8.113 | 26.301 | 1.00 | 12.83 |
| ATOM | 341 | C | PHE | A | 579 | −6.040 | 8.170 | 23.708 | 1.00 | 12.84 |
| ATOM | 342 | O | PHE | A | 579 | −6.765 | 7.429 | 24.367 | 1.00 | 12.73 |
| ATOM | 343 | N | THR | A | 580 | −6.504 | 9.151 | 22.943 | 1.00 | 15.04 |
| ATOM | 344 | CA | THR | A | 580 | −7.926 | 9.459 | 22.839 | 1.00 | 14.30 |
| ATOM | 345 | CB | THR | A | 580 | −8.145 | 10.702 | 21.967 | 1.00 | 10.78 |
| ATOM | 346 | OG1 | THR | A | 580 | −7.344 | 11.780 | 22.469 | 1.00 | 18.08 |
| ATOM | 347 | CG2 | THR | A | 580 | −9.610 | 11.115 | 21.987 | 1.00 | 13.16 |
| ATOM | 348 | C | THR | A | 580 | −8.772 | 8.327 | 22.273 | 1.00 | 14.34 |
| ATOM | 349 | O | THR | A | 580 | −9.774 | 7.936 | 22.871 | 1.00 | 14.67 |
| ATOM | 350 | N | ASP | A | 581 | −8.367 | 7.806 | 21.119 | 1.00 | 15.75 |
| ATOM | 351 | CA | ASP | A | 581 | −9.106 | 6.731 | 20.466 | 1.00 | 14.61 |
| ATOM | 352 | CB | ASP | A | 581 | −8.724 | 6.668 | 18.990 | 1.00 | 16.27 |
| ATOM | 353 | CG | ASP | A | 581 | −9.398 | 7.757 | 18.181 | 1.00 | 20.38 |
| ATOM | 354 | OD1 | ASP | A | 581 | −9.762 | 8.797 | 18.776 | 1.00 | 15.70 |
| ATOM | 355 | OD2 | ASP | A | 581 | −9.560 | 7.580 | 16.958 | 1.00 | 17.57 |
| ATOM | 356 | C | ASP | A | 581 | −8.970 | 5.365 | 21.123 | 1.00 | 15.19 |
| ATOM | 357 | O | ASP | A | 581 | −9.627 | 4.402 | 20.719 | 1.00 | 14.62 |
| ATOM | 358 | N | LEU | A | 582 | −8.110 | 5.275 | 22.130 | 1.00 | 13.55 |
| ATOM | 359 | CA | LEU | A | 582 | −7.957 | 4.032 | 22.867 | 1.00 | 14.36 |
| ATOM | 360 | CB | LEU | A | 582 | −6.484 | 3.739 | 23.156 | 1.00 | 16.24 |
| ATOM | 361 | CG | LEU | A | 582 | −5.770 | 3.024 | 22.006 | 1.00 | 14.56 |
| ATOM | 362 | CD1 | LEU | A | 582 | −4.272 | 2.968 | 22.250 | 1.00 | 12.82 |
| ATOM | 363 | CD2 | LEU | A | 582 | −6.351 | 1.625 | 21.865 | 1.00 | 12.05 |
| ATOM | 364 | C | LEU | A | 582 | −8.739 | 4.189 | 24.168 | 1.00 | 17.31 |
| ATOM | 365 | O | LEU | A | 582 | −8.617 | 3.378 | 25.081 | 1.00 | 17.82 |
| ATOM | 366 | N | ASN | A | 583 | −9.533 | 5.255 | 24.246 | 1.00 | 17.93 |
| ATOM | 367 | CA | ASN | A | 583 | −10.361 | 5.500 | 25.420 | 1.00 | 21.01 |
| ATOM | 368 | CB | ASN | A | 583 | −11.405 | 4.377 | 25.525 | 1.00 | 22.07 |
| ATOM | 369 | CG | ASN | A | 583 | −12.368 | 4.564 | 26.682 | 1.00 | 34.95 |
| ATOM | 370 | OD1 | ASN | A | 583 | −12.921 | 5.645 | 26.879 | 1.00 | 34.93 |
| ATOM | 371 | ND2 | ASN | A | 583 | −12.589 | 3.496 | 27.444 | 1.00 | 39.94 |
| ATOM | 372 | C | ASN | A | 583 | −9.516 | 5.575 | 26.693 | 1.00 | 23.16 |
| ATOM | 373 | O | ASN | A | 583 | −9.866 | 4.978 | 27.713 | 1.00 | 27.41 |
| ATOM | 374 | N | LEU | A | 584 | −8.406 | 6.313 | 26.634 | 1.00 | 18.24 |
| ATOM | 375 | CA | LEU | A | 584 | −7.517 | 6.446 | 27.791 | 1.00 | 18.39 |
| ATOM | 376 | CB | LEU | A | 584 | −6.060 | 6.188 | 27.379 | 1.00 | 13.74 |
| ATOM | 377 | CG | LEU | A | 584 | −5.713 | 4.823 | 26.778 | 1.00 | 13.61 |
| ATOM | 378 | CD1 | LEU | A | 584 | −4.226 | 4.789 | 26.412 | 1.00 | 16.18 |
| ATOM | 379 | CD2 | LEU | A | 584 | −6.050 | 3.726 | 27.769 | 1.00 | 15.78 |
| ATOM | 380 | C | LEU | A | 584 | −7.591 | 7.807 | 28.486 | 1.00 | 18.85 |
| ATOM | 381 | O | LEU | A | 584 | −7.377 | 7.904 | 29.695 | 1.00 | 18.54 |
| ATOM | 382 | N | VAL | A | 585 | −7.890 | 8.853 | 27.721 | 1.00 | 18.53 |
| ATOM | 383 | CA | VAL | A | 585 | −7.956 | 10.211 | 28.258 | 1.00 | 20.90 |
| ATOM | 384 | CB | VAL | A | 585 | −8.157 | 11.234 | 27.123 | 1.00 | 25.63 |
| ATOM | 385 | CG1 | VAL | A | 585 | −8.083 | 12.648 | 27.672 | 1.00 | 26.57 |
| ATOM | 386 | CG2 | VAL | A | 585 | −7.102 | 11.024 | 26.054 | 1.00 | 26.78 |
| ATOM | 387 | C | VAL | A | 585 | −9.049 | 10.416 | 29.307 | 1.00 | 21.72 |
| ATOM | 388 | O | VAL | A | 585 | −8.771 | 10.839 | 30.429 | 1.00 | 21.94 |
| ATOM | 389 | N | GLN | A | 586 | −10.291 | 10.118 | 28.945 | 1.00 | 22.79 |
| ATOM | 390 | CA | GLN | A | 586 | −11.394 | 10.290 | 29.881 | 1.00 | 25.11 |
| ATOM | 391 | CB | GLN | A | 586 | −12.728 | 10.306 | 29.131 | 1.00 | 34.29 |
| ATOM | 392 | CG | GLN | A | 586 | −13.719 | 11.317 | 29.678 | 1.00 | 39.65 |
| ATOM | 393 | CD | GLN | A | 586 | −13.155 | 12.725 | 29.663 | 1.00 | 50.17 |
| ATOM | 394 | OE1 | GLN | A | 586 | −12.715 | 13.218 | 28.622 | 1.00 | 52.20 |
| ATOM | 395 | NE2 | GLN | A | 586 | −13.160 | 13.380 | 30.820 | 1.00 | 48.68 |
| ATOM | 396 | C | GLN | A | 586 | −11.402 | 9.172 | 30.914 | 1.00 | 22.36 |
| ATOM | 397 | O | GLN | A | 586 | −11.587 | 9.413 | 32.108 | 1.00 | 23.81 |
| ATOM | 398 | N | ASN | A | 587 | −11.195 | 7.948 | 30.441 | 1.00 | 22.47 |
| ATOM | 399 | CA | ASN | A | 587 | −11.181 | 6.769 | 31.298 | 1.00 | 19.07 |
| ATOM | 400 | CB | ASN | A | 587 | −10.651 | 5.565 | 30.507 | 1.00 | 21.06 |
| ATOM | 401 | CG | ASN | A | 587 | −10.944 | 4.236 | 31.179 | 1.00 | 29.14 |
| ATOM | 402 | OD1 | ASN | A | 587 | −11.098 | 4.157 | 32.397 | 1.00 | 21.26 |
| ATOM | 403 | ND2 | ASN | A | 587 | −11.002 | 3.174 | 30.382 | 1.00 | 33.44 |
| ATOM | 404 | C | ASN | A | 587 | −10.319 | 6.981 | 32.542 | 1.00 | 20.86 |
| ATOM | 405 | O | ASN | A | 587 | −10.727 | 6.646 | 33.656 | 1.00 | 19.43 |
| ATOM | 406 | N | PHE | A | 588 | −9.134 | 7.553 | 32.357 | 1.00 | 18.57 |
| ATOM | 407 | CA | PHE | A | 588 | −8.227 | 7.753 | 33.480 | 1.00 | 18.65 |
| ATOM | 408 | CB | PHE | A | 588 | −6.880 | 7.109 | 33.145 | 1.00 | 13.88 |
| ATOM | 409 | CG | PHE | A | 588 | −6.976 | 5.624 | 32.948 | 1.00 | 14.75 |
| ATOM | 410 | CD1 | PHE | A | 588 | −7.272 | 4.791 | 34.022 | 1.00 | 13.58 |
| ATOM | 411 | CD2 | PHE | A | 588 | −6.860 | 5.065 | 31.686 | 1.00 | 12.05 |
| ATOM | 412 | CE1 | PHE | A | 588 | −7.456 | 3.431 | 33.837 | 1.00 | 13.16 |
| ATOM | 413 | CE2 | PHE | A | 588 | −7.044 | 3.704 | 31.496 | 1.00 | 10.45 |
| ATOM | 414 | CZ | PHE | A | 588 | −7.344 | 2.888 | 32.576 | 1.00 | 16.21 |
| ATOM | 415 | C | PHE | A | 588 | −8.063 | 9.190 | 33.966 | 1.00 | 20.57 |
| ATOM | 416 | O | PHE | A | 588 | −7.072 | 9.538 | 34.611 | 1.00 | 18.90 |
| ATOM | 417 | N | GLN | A | 589 | −9.052 | 10.020 | 33.662 | 1.00 | 21.41 |
| ATOM | 418 | CA | GLN | A | 589 | −9.057 | 11.410 | 34.110 | 1.00 | 23.36 |
| ATOM | 419 | CB | GLN | A | 589 | −9.213 | 11.449 | 35.634 | 1.00 | 28.73 |
| ATOM | 420 | CG | GLN | A | 589 | −10.311 | 10.551 | 36.194 | 1.00 | 40.43 |
| ATOM | 421 | CD | GLN | A | 589 | −11.701 | 11.003 | 35.797 | 1.00 | 42.17 |
| ATOM | 422 | OE1 | GLN | A | 589 | −12.058 | 12.167 | 35.971 | 1.00 | 38.12 |
| ATOM | 423 | NE2 | GLN | A | 589 | −12.498 | 10.080 | 35.270 | 1.00 | 47.35 |
| ATOM | 424 | C | GLN | A | 589 | −7.805 | 12.201 | 33.725 | 1.00 | 20.90 |
| ATOM | 425 | O | GLN | A | 589 | −7.282 | 12.972 | 34.531 | 1.00 | 21.16 |
| ATOM | 426 | N | MET | A | 590 | −7.321 | 12.019 | 32.502 | 1.00 | 22.12 |
| ATOM | 427 | CA | MET | A | 590 | −6.133 | 12.745 | 32.069 | 1.00 | 21.73 |
| ATOM | 428 | CB | MET | A | 590 | −5.463 | 12.035 | 30.893 | 1.00 | 22.93 |
| ATOM | 429 | CG | MET | A | 590 | −4.751 | 10.752 | 31.257 | 1.00 | 24.06 |
| ATOM | 430 | SD | MET | A | 590 | −3.865 | 10.087 | 29.838 | 1.00 | 19.72 |
| ATOM | 431 | CE | MET | A | 590 | −3.631 | 8.404 | 30.357 | 1.00 | 17.35 |
| ATOM | 432 | C | MET | A | 590 | −6.455 | 14.172 | 31.654 | 1.00 | 22.68 |
| ATOM | 433 | O | MET | A | 590 | −7.295 | 14.398 | 30.784 | 1.00 | 23.69 |
| ATOM | 434 | N | LYS | A | 591 | −5.792 | 15.138 | 32.278 | 1.00 | 20.35 |
| ATOM | 435 | CA | LYS | A | 591 | −6.013 | 16.530 | 31.919 | 1.00 | 20.85 |
| ATOM | 436 | CB | LYS | A | 591 | −5.554 | 17.460 | 33.045 | 1.00 | 22.95 |
| ATOM | 437 | CG | LYS | A | 591 | −6.472 | 17.422 | 34.262 | 1.00 | 28.61 |
| ATOM | 438 | CD | LYS | A | 591 | −6.065 | 18.438 | 35.315 | 1.00 | 37.59 |
| ATOM | 439 | CE | LYS | A | 591 | −7.106 | 18.527 | 36.423 | 1.00 | 42.92 |
| ATOM | 440 | NZ | LYS | A | 591 | −8.417 | 19.026 | 35.915 | 1.00 | 51.43 |
| ATOM | 441 | C | LYS | A | 591 | −5.235 | 16.800 | 30.636 | 1.00 | 20.72 |
| ATOM | 442 | O | LYS | A | 591 | −4.048 | 16.486 | 30.537 | 1.00 | 21.29 |
| ATOM | 443 | N | HIS | A | 592 | −5.920 | 17.375 | 29.654 | 1.00 | 20.84 |
| ATOM | 444 | CA | HIS | A | 592 | −5.333 | 17.670 | 28.351 | 1.00 | 21.11 |
| ATOM | 445 | CB | HIS | A | 592 | −6.273 | 18.577 | 27.553 | 1.00 | 21.39 |
| ATOM | 446 | CG | HIS | A | 592 | −5.884 | 18.737 | 26.125 | 1.00 | 18.05 |
| ATOM | 447 | CD2 | HIS | A | 592 | −5.427 | 19.798 | 25.424 | 1.00 | 24.75 |
| ATOM | 448 | ND1 | HIS | A | 592 | −5.926 | 17.685 | 25.213 | 1.00 | 22.74 |
| ATOM | 449 | CE1 | HIS | A | 592 | −5.518 | 18.102 | 24.044 | 1.00 | 29.20 |
| ATOM | 450 | NE2 | HIS | A | 592 | −5.204 | 19.393 | 24.135 | 1.00 | 27.94 |
| ATOM | 451 | C | HIS | A | 592 | −3.937 | 18.295 | 28.377 | 1.00 | 20.49 |
| ATOM | 452 | O | HIS | A | 592 | −3.034 | 17.848 | 27.661 | 1.00 | 19.49 |
| ATOM | 453 | N | GLU | A | 593 | −3.765 | 19.338 | 29.184 | 1.00 | 19.12 |
| ATOM | 454 | CA | GLU | A | 593 | −2.481 | 20.019 | 29.279 | 1.00 | 18.26 |
| ATOM | 455 | CB | GLU | A | 593 | −2.592 | 21.243 | 30.192 | 1.00 | 23.02 |
| ATOM | 456 | CG | GLU | A | 593 | −1.267 | 21.957 | 30.416 | 1.00 | 32.52 |
| ATOM | 457 | CD | GLU | A | 593 | −1.422 | 23.296 | 31.116 | 1.00 | 44.01 |
| ATOM | 458 | OE1 | GLU | A | 593 | −2.062 | 24.200 | 30.535 | 1.00 | 41.08 |
| ATOM | 459 | OE2 | GLU | A | 593 | −0.901 | 23.446 | 32.244 | 1.00 | 45.85 |
| ATOM | 460 | C | GLU | A | 593 | −1.381 | 19.099 | 29.793 | 1.00 | 18.42 |
| ATOM | 461 | O | GLU | A | 593 | −0.230 | 19.195 | 29.365 | 1.00 | 16.87 |
| ATOM | 462 | N | VAL | A | 594 | −1.738 | 18.209 | 30.710 | 1.00 | 18.22 |
| ATOM | 463 | CA | VAL | A | 594 | −0.770 | 17.275 | 31.277 | 1.00 | 17.13 |
| ATOM | 464 | CB | VAL | A | 594 | −1.372 | 16.506 | 32.465 | 1.00 | 16.16 |
| ATOM | 465 | CG1 | VAL | A | 594 | −0.314 | 15.605 | 33.085 | 1.00 | 20.37 |
| ATOM | 466 | CG2 | VAL | A | 594 | −1.911 | 17.481 | 33.491 | 1.00 | 22.54 |
| ATOM | 467 | C | VAL | A | 594 | −0.299 | 16.263 | 30.235 | 1.00 | 15.25 |
| ATOM | 468 | O | VAL | A | 594 | 0.902 | 16.005 | 30.101 | 1.00 | 16.26 |
| ATOM | 469 | N | LEU | A | 595 | −1.246 | 15.681 | 29.504 | 1.00 | 13.83 |
| ATOM | 470 | CA | LEU | A | 595 | −0.897 | 14.705 | 28.478 | 1.00 | 14.01 |
| ATOM | 471 | CB | LEU | A | 595 | −2.163 | 14.147 | 27.818 | 1.00 | 9.50 |
| ATOM | 472 | CG | LEU | A | 595 | −1.969 | 13.167 | 26.653 | 1.00 | 18.45 |
| ATOM | 473 | CD1 | LEU | A | 595 | −1.068 | 12.014 | 27.076 | 1.00 | 12.38 |
| ATOM | 474 | CD2 | LEU | A | 595 | −3.318 | 12.643 | 26.201 | 1.00 | 14.12 |
| ATOM | 475 | C | LEU | A | 595 | −0.002 | 15.372 | 27.438 | 1.00 | 13.61 |
| ATOM | 476 | O | LEU | A | 595 | 1.004 | 14.804 | 27.010 | 1.00 | 13.98 |
| ATOM | 477 | N | CYS | A | 596 | −0.358 | 16.588 | 27.037 | 1.00 | 14.70 |
| ATOM | 478 | CA | CYS | A | 596 | 0.448 | 17.303 | 26.058 | 1.00 | 12.24 |
| ATOM | 479 | CB | CYS | A | 596 | −0.191 | 18.649 | 25.709 | 1.00 | 15.21 |
| ATOM | 480 | SG | CYS | A | 596 | −1.702 | 18.507 | 24.749 | 1.00 | 17.45 |
| ATOM | 481 | C | CYS | A | 596 | 1.861 | 17.534 | 26.586 | 1.00 | 12.70 |
| ATOM | 482 | O | CYS | A | 596 | 2.839 | 17.327 | 25.868 | 1.00 | 11.06 |
| ATOM | 483 | N | ARG | A | 597 | 1.970 | 17.959 | 27.841 | 1.00 | 11.36 |
| ATOM | 484 | CA | ARG | A | 597 | 3.283 | 18.211 | 28.420 | 1.00 | 11.51 |
| ATOM | 485 | CB | ARG | A | 597 | 3.141 | 18.836 | 29.815 | 1.00 | 14.74 |
| ATOM | 486 | CG | ARG | A | 597 | 4.463 | 19.343 | 30.388 | 1.00 | 15.86 |
| ATOM | 487 | CD | ARG | A | 597 | 4.294 | 20.067 | 31.727 | 1.00 | 20.22 |
| ATOM | 488 | NE | ARG | A | 597 | 3.565 | 21.332 | 31.614 | 1.00 | 21.93 |
| ATOM | 489 | CZ | ARG | A | 597 | 2.279 | 21.493 | 31.916 | 1.00 | 27.00 |
| ATOM | 490 | NH1 | ARG | A | 597 | 1.560 | 20.469 | 32.357 | 1.00 | 22.77 |
| ATOM | 491 | NH2 | ARG | A | 597 | 1.708 | 22.683 | 31.782 | 1.00 | 30.27 |
| ATOM | 492 | C | ARG | A | 597 | 4.100 | 16.919 | 28.494 | 1.00 | 11.80 |
| ATOM | 493 | O | ARG | A | 597 | 5.287 | 16.909 | 28.162 | 1.00 | 10.48 |
| ATOM | 494 | N | TRP | A | 598 | 3.454 | 15.830 | 28.905 | 1.00 | 11.97 |
| ATOM | 495 | CA | TRP | A | 598 | 4.112 | 14.530 | 29.022 | 1.00 | 9.62 |
| ATOM | 496 | CB | TRP | A | 598 | 3.125 | 13.488 | 29.556 | 1.00 | 11.57 |
| ATOM | 497 | CG | TRP | A | 598 | 3.717 | 12.110 | 29.702 | 1.00 | 10.65 |
| ATOM | 498 | CD2 | TRP | A | 598 | 3.609 | 11.028 | 28.771 | 1.00 | 9.66 |
| ATOM | 499 | CE2 | TRP | A | 598 | 4.350 | 9.948 | 29.298 | 1.00 | 8.24 |
| ATOM | 500 | CE3 | TRP | A | 598 | 2.964 | 10.866 | 27.541 | 1.00 | 11.87 |
| ATOM | 501 | CD1 | TRP | A | 598 | 4.494 | 11.656 | 30.728 | 1.00 | 9.74 |
| ATOM | 502 | NE1 | TRP | A | 598 | 4.878 | 10.359 | 30.492 | 1.00 | 11.42 |
| ATOM | 503 | CZ2 | TRP | A | 598 | 4.459 | 8.720 | 28.635 | 1.00 | 7.38 |
| ATOM | 504 | CZ3 | TRP | A | 598 | 3.075 | 9.646 | 26.886 | 1.00 | 9.43 |
| ATOM | 505 | CH2 | TRP | A | 598 | 3.818 | 8.593 | 27.434 | 1.00 | 10.02 |
| ATOM | 506 | C | TRP | A | 598 | 4.656 | 14.064 | 27.670 | 1.00 | 12.66 |
| ATOM | 507 | O | TRP | A | 598 | 5.804 | 13.617 | 27.568 | 1.00 | 11.33 |
| ATOM | 508 | N | ILE | A | 599 | 3.823 | 14.160 | 26.637 | 1.00 | 9.84 |
| ATOM | 509 | CA | ILE | A | 599 | 4.232 | 13.748 | 25.298 | 1.00 | 12.03 |
| ATOM | 510 | CB | ILE | A | 599 | 3.101 | 13.974 | 24.270 | 1.00 | 11.30 |
| ATOM | 511 | CG2 | ILE | A | 599 | 3.648 | 13.807 | 22.856 | 1.00 | 13.41 |
| ATOM | 512 | CG1 | ILE | A | 599 | 1.953 | 13.000 | 24.546 | 1.00 | 13.16 |
| ATOM | 513 | CD1 | ILE | A | 599 | 0.732 | 13.218 | 23.668 | 1.00 | 13.06 |
| ATOM | 514 | C | ILE | A | 599 | 5.460 | 14.533 | 24.855 | 1.00 | 14.21 |
| ATOM | 515 | O | ILE | A | 599 | 6.414 | 13.969 | 24.308 | 1.00 | 11.47 |
| ATOM | 516 | N | LEU | A | 600 | 5.439 | 15.839 | 25.094 | 1.00 | 14.33 |
| ATOM | 517 | CA | LEU | A | 600 | 6.566 | 16.670 | 24.707 | 1.00 | 13.61 |
| ATOM | 518 | CB | LEU | A | 600 | 6.214 | 18.151 | 24.882 | 1.00 | 13.53 |
| ATOM | 519 | CG | LEU | A | 600 | 5.073 | 18.621 | 23.971 | 1.00 | 12.80 |
| ATOM | 520 | CD1 | LEU | A | 600 | 4.652 | 20.036 | 24.336 | 1.00 | 15.66 |
| ATOM | 521 | CD2 | LEU | A | 600 | 5.518 | 18.557 | 22.520 | 1.00 | 13.09 |
| ATOM | 522 | C | LEU | A | 600 | 7.800 | 16.297 | 25.523 | 1.00 | 10.78 |
| ATOM | 523 | O | LEU | A | 600 | 8.917 | 16.324 | 25.011 | 1.00 | 10.47 |
| ATOM | 524 | N | SER | A | 601 | 7.601 | 15.925 | 26.788 | 1.00 | 10.50 |
| ATOM | 525 | CA | SER | A | 601 | 8.734 | 15.540 | 27.624 | 1.00 | 8.54 |
| ATOM | 526 | CB | SER | A | 601 | 8.303 | 15.398 | 29.085 | 1.00 | 9.00 |
| ATOM | 527 | OG | SER | A | 601 | 8.036 | 16.678 | 29.634 | 1.00 | 9.87 |
| ATOM | 528 | C | SER | A | 601 | 9.342 | 14.239 | 27.129 | 1.00 | 9.99 |
| ATOM | 529 | O | SER | A | 601 | 10.563 | 14.082 | 27.104 | 1.00 | 10.43 |
| ATOM | 530 | N | VAL | A | 602 | 8.487 | 13.300 | 26.739 | 1.00 | 10.77 |
| ATOM | 531 | CA | VAL | A | 602 | 8.968 | 12.024 | 26.223 | 1.00 | 10.98 |
| ATOM | 532 | CB | VAL | A | 602 | 7.791 | 11.081 | 25.874 | 1.00 | 12.52 |
| ATOM | 533 | CG1 | VAL | A | 602 | 8.303 | 9.856 | 25.138 | 1.00 | 9.22 |
| ATOM | 534 | CG2 | VAL | A | 602 | 7.068 | 10.658 | 27.152 | 1.00 | 11.71 |
| ATOM | 535 | C | VAL | A | 602 | 9.794 | 12.290 | 24.967 | 1.00 | 12.57 |
| ATOM | 536 | O | VAL | A | 602 | 10.935 | 11.848 | 24.855 | 1.00 | 11.99 |
| ATOM | 537 | N | LYS | A | 603 | 9.220 | 13.043 | 24.034 | 1.00 | 12.97 |
| ATOM | 538 | CA | LYS | A | 603 | 9.906 | 13.363 | 22.792 | 1.00 | 11.73 |
| ATOM | 539 | CB | LYS | A | 603 | 9.025 | 14.268 | 21.928 | 1.00 | 14.00 |
| ATOM | 540 | CG | LYS | A | 603 | 9.666 | 14.688 | 20.620 | 1.00 | 14.56 |
| ATOM | 541 | CD | LYS | A | 603 | 8.656 | 15.317 | 19.686 | 1.00 | 16.82 |
| ATOM | 542 | CE | LYS | A | 603 | 8.088 | 16.603 | 20.262 | 1.00 | 24.97 |
| ATOM | 543 | NZ | LYS | A | 603 | 7.169 | 17.256 | 19.300 | 1.00 | 39.23 |
| ATOM | 544 | C | LYS | A | 603 | 11.256 | 14.035 | 23.042 | 1.00 | 11.50 |
| ATOM | 545 | O | LYS | A | 603 | 12.252 | 13.694 | 22.414 | 1.00 | 14.04 |
| ATOM | 546 | N | LYS | A | 604 | 11.279 | 14.985 | 23.968 | 1.00 | 11.12 |
| ATOM | 547 | CA | LYS | A | 604 | 12.501 | 15.714 | 24.293 | 1.00 | 13.84 |
| ATOM | 548 | CB | LYS | A | 604 | 12.181 | 16.849 | 25.274 | 1.00 | 16.59 |
| ATOM | 549 | CG | LYS | A | 604 | 13.403 | 17.589 | 25.788 | 1.00 | 18.98 |
| ATOM | 550 | CD | LYS | A | 604 | 13.016 | 18.673 | 26.784 | 1.00 | 25.53 |
| ATOM | 551 | CE | LYS | A | 604 | 14.251 | 19.335 | 27.391 | 1.00 | 31.51 |
| ATOM | 552 | NZ | LYS | A | 604 | 13.893 | 20.351 | 28.424 | 1.00 | 29.80 |
| ATOM | 553 | C | LYS | A | 604 | 13.582 | 14.824 | 24.890 | 1.00 | 12.98 |
| ATOM | 554 | O | LYS | A | 604 | 14.776 | 15.073 | 24.710 | 1.00 | 13.10 |
| ATOM | 555 | N | ASN | A | 605 | 13.173 | 13.773 | 25.587 | 1.00 | 12.62 |
| ATOM | 556 | CA | ASN | A | 605 | 14.146 | 12.901 | 26.220 | 1.00 | 13.04 |
| ATOM | 557 | CB | ASN | A | 605 | 13.555 | 12.336 | 27.507 | 1.00 | 13.13 |
| ATOM | 558 | CG | ASN | A | 605 | 13.579 | 13.356 | 28.638 | 1.00 | 32.58 |
| ATOM | 559 | OD1 | ASN | A | 605 | 14.638 | 13.650 | 29.197 | 1.00 | 25.76 |
| ATOM | 560 | ND2 | ASN | A | 605 | 12.418 | 13.918 | 28.962 | 1.00 | 30.95 |
| ATOM | 561 | C | ASN | A | 605 | 14.757 | 11.809 | 25.352 | 1.00 | 15.12 |
| ATOM | 562 | O | ASN | A | 605 | 15.342 | 10.848 | 25.858 | 1.00 | 18.48 |
| ATOM | 563 | N | TYR | A | 606 | 14.612 | 11.961 | 24.042 | 1.00 | 10.60 |
| ATOM | 564 | CA | TYR | A | 606 | 15.233 | 11.051 | 23.093 | 1.00 | 10.45 |
| ATOM | 565 | CB | TYR | A | 606 | 14.254 | 10.645 | 21.982 | 1.00 | 10.34 |
| ATOM | 566 | CG | TYR | A | 606 | 13.420 | 9.444 | 22.358 | 1.00 | 11.02 |
| ATOM | 567 | CD1 | TYR | A | 606 | 13.950 | 8.156 | 22.297 | 1.00 | 8.31 |
| ATOM | 568 | CE1 | TYR | A | 606 | 13.222 | 7.061 | 22.752 | 1.00 | 8.28 |
| ATOM | 569 | CD2 | TYR | A | 606 | 12.139 | 9.601 | 22.874 | 1.00 | 12.47 |
| ATOM | 570 | CE2 | TYR | A | 606 | 11.410 | 8.516 | 23.335 | 1.00 | 15.05 |
| ATOM | 571 | CZ | TYR | A | 606 | 11.955 | 7.252 | 23.275 | 1.00 | 15.46 |
| ATOM | 572 | OH | TYR | A | 606 | 11.227 | 6.188 | 23.773 | 1.00 | 12.36 |
| ATOM | 573 | C | TYR | A | 606 | 16.362 | 11.908 | 22.539 | 1.00 | 11.41 |
| ATOM | 574 | O | TYR | A | 606 | 16.216 | 13.129 | 22.428 | 1.00 | 14.91 |
| ATOM | 575 | N | ARG | A | 607 | 17.499 | 11.293 | 22.234 | 1.00 | 11.41 |
| ATOM | 576 | CA | ARG | A | 607 | 18.627 | 12.042 | 21.700 | 1.00 | 10.90 |
| ATOM | 577 | CB | ARG | A | 607 | 19.933 | 11.322 | 22.034 | 1.00 | 12.68 |
| ATOM | 578 | CG | ARG | A | 607 | 20.078 | 11.019 | 23.520 | 1.00 | 10.36 |
| ATOM | 579 | CD | ARG | A | 607 | 21.460 | 10.489 | 23.870 | 1.00 | 13.25 |
| ATOM | 580 | NE | ARG | A | 607 | 21.492 | 9.929 | 25.221 | 1.00 | 9.55 |
| ATOM | 581 | CZ | ARG | A | 607 | 22.601 | 9.556 | 25.854 | 1.00 | 18.33 |
| ATOM | 582 | NH1 | ARG | A | 607 | 23.781 | 9.686 | 25.262 | 1.00 | 13.50 |
| ATOM | 583 | NH2 | ARG | A | 607 | 22.530 | 9.045 | 27.077 | 1.00 | 17.68 |
| ATOM | 584 | C | ARG | A | 607 | 18.425 | 12.150 | 20.194 | 1.00 | 10.45 |
| ATOM | 585 | O | ARG | A | 607 | 18.616 | 11.182 | 19.458 | 1.00 | 12.53 |
| ATOM | 586 | N | LYS | A | 608 | 18.052 | 13.340 | 19.738 | 1.00 | 11.88 |
| ATOM | 587 | CA | LYS | A | 608 | 17.755 | 13.555 | 18.323 | 1.00 | 13.33 |
| ATOM | 588 | CB | LYS | A | 608 | 17.273 | 14.993 | 18.104 | 1.00 | 18.33 |
| ATOM | 589 | CG | LYS | A | 608 | 18.226 | 16.064 | 18.574 | 1.00 | 34.41 |
| ATOM | 590 | CD | LYS | A | 608 | 17.558 | 17.429 | 18.538 | 1.00 | 39.16 |
| ATOM | 591 | CE | LYS | A | 608 | 18.430 | 18.485 | 19.193 | 1.00 | 44.56 |
| ATOM | 592 | NZ | LYS | A | 608 | 19.755 | 18.595 | 18.525 | 1.00 | 49.34 |
| ATOM | 593 | C | LYS | A | 608 | 18.837 | 13.220 | 17.311 | 1.00 | 13.41 |
| ATOM | 594 | O | LYS | A | 608 | 18.527 | 12.904 | 16.164 | 1.00 | 14.71 |
| ATOM | 595 | N | ASN | A | 609 | 20.098 | 13.272 | 17.724 | 1.00 | 12.65 |
| ATOM | 596 | CA | ASN | A | 609 | 21.185 | 12.977 | 16.804 | 1.00 | 15.37 |
| ATOM | 597 | CB | ASN | A | 609 | 22.383 | 13.872 | 17.121 | 1.00 | 19.55 |
| ATOM | 598 | CG | ASN | A | 609 | 22.059 | 15.347 | 16.952 | 1.00 | 21.29 |
| ATOM | 599 | OD1 | ASN | A | 609 | 21.630 | 15.779 | 15.882 | 1.00 | 28.72 |
| ATOM | 600 | ND2 | ASN | A | 609 | 22.261 | 16.126 | 18.009 | 1.00 | 36.76 |
| ATOM | 601 | C | ASN | A | 609 | 21.593 | 11.507 | 16.743 | 1.00 | 14.47 |
| ATOM | 602 | O | ASN | A | 609 | 22.486 | 11.135 | 15.981 | 1.00 | 15.29 |
| ATOM | 603 | N | VAL | A | 610 | 20.955 | 10.670 | 17.557 | 1.00 | 11.89 |
| ATOM | 604 | CA | VAL | A | 610 | 21.221 | 9.233 | 17.511 | 1.00 | 11.43 |
| ATOM | 605 | CB | VAL | A | 610 | 20.696 | 8.531 | 18.777 | 1.00 | 11.38 |
| ATOM | 606 | CG1 | VAL | A | 610 | 20.649 | 7.019 | 18.571 | 1.00 | 10.56 |
| ATOM | 607 | CG2 | VAL | A | 610 | 21.608 | 8.882 | 19.955 | 1.00 | 10.47 |
| ATOM | 608 | C | VAL | A | 610 | 20.432 | 8.809 | 16.272 | 1.00 | 12.97 |
| ATOM | 609 | O | VAL | A | 610 | 19.221 | 9.025 | 16.198 | 1.00 | 11.59 |
| ATOM | 610 | N | ALA | A | 611 | 21.131 | 8.223 | 15.302 | 1.00 | 11.37 |
| ATOM | 611 | CA | ALA | A | 611 | 20.554 | 7.833 | 14.018 | 1.00 | 12.77 |
| ATOM | 612 | CB | ALA | A | 611 | 21.579 | 7.021 | 13.223 | 1.00 | 12.83 |
| ATOM | 613 | C | ALA | A | 611 | 19.208 | 7.117 | 13.981 | 1.00 | 12.88 |
| ATOM | 614 | O | ALA | A | 611 | 18.305 | 7.522 | 13.247 | 1.00 | 14.79 |
| ATOM | 615 | N | TYR | A | 612 | 19.071 | 6.050 | 14.756 | 1.00 | 12.18 |
| ATOM | 616 | CA | TYR | A | 612 | 17.830 | 5.288 | 14.743 | 1.00 | 10.69 |
| ATOM | 617 | CB | TYR | A | 612 | 18.147 | 3.824 | 14.408 | 1.00 | 13.15 |
| ATOM | 618 | CG | TYR | A | 612 | 16.968 | 2.877 | 14.509 | 1.00 | 12.63 |
| ATOM | 619 | CD1 | TYR | A | 612 | 15.832 | 3.054 | 13.729 | 1.00 | 19.45 |
| ATOM | 620 | CE1 | TYR | A | 612 | 14.752 | 2.185 | 13.833 | 1.00 | 24.00 |
| ATOM | 621 | CD2 | TYR | A | 612 | 16.996 | 1.805 | 15.392 | 1.00 | 13.47 |
| ATOM | 622 | CE2 | TYR | A | 612 | 15.930 | 0.938 | 15.503 | 1.00 | 24.95 |
| ATOM | 623 | CZ | TYR | A | 612 | 14.812 | 1.129 | 14.722 | 1.00 | 24.27 |
| ATOM | 624 | OH | TYR | A | 612 | 13.761 | 0.245 | 14.831 | 1.00 | 25.94 |
| ATOM | 625 | C | TYR | A | 612 | 17.017 | 5.371 | 16.028 | 1.00 | 11.05 |
| ATOM | 626 | O | TYR | A | 612 | 15.812 | 5.632 | 15.989 | 1.00 | 9.25 |
| ATOM | 627 | N | HIS | A | 613 | 17.667 | 5.159 | 17.166 | 1.00 | 10.71 |
| ATOM | 628 | CA | HIS | A | 613 | 16.951 | 5.199 | 18.432 | 1.00 | 9.46 |
| ATOM | 629 | CB | HIS | A | 613 | 17.694 | 4.391 | 19.499 | 1.00 | 12.29 |
| ATOM | 630 | CG | HIS | A | 613 | 17.706 | 2.923 | 19.232 | 1.00 | 13.22 |
| ATOM | 631 | CD2 | HIS | A | 613 | 16.742 | 1.983 | 19.364 | 1.00 | 13.53 |
| ATOM | 632 | ND1 | HIS | A | 613 | 18.811 | 2.262 | 18.726 | 1.00 | 8.83 |
| ATOM | 633 | CE1 | HIS | A | 613 | 18.523 | 0.989 | 18.562 | 1.00 | 12.86 |
| ATOM | 634 | NE2 | HIS | A | 613 | 17.268 | 0.789 | 18.942 | 1.00 | 16.65 |
| ATOM | 635 | C | HIS | A | 613 | 16.686 | 6.606 | 18.936 | 1.00 | 11.81 |
| ATOM | 636 | O | HIS | A | 613 | 17.226 | 7.023 | 19.957 | 1.00 | 11.26 |
| ATOM | 637 | N | ASN | A | 614 | 15.859 | 7.333 | 18.192 | 1.00 | 12.20 |
| ATOM | 638 | CA | ASN | A | 614 | 15.459 | 8.685 | 18.556 | 1.00 | 11.65 |
| ATOM | 639 | CB | ASN | A | 614 | 15.955 | 9.702 | 17.516 | 1.00 | 19.08 |
| ATOM | 640 | CG | ASN | A | 614 | 15.631 | 9.295 | 16.090 | 1.00 | 17.63 |
| ATOM | 641 | OD1 | ASN | A | 614 | 16.529 | 9.151 | 15.253 | 1.00 | 16.40 |
| ATOM | 642 | ND2 | ASN | A | 614 | 14.349 | 9.115 | 15.802 | 1.00 | 11.39 |
| ATOM | 643 | C | ASN | A | 614 | 13.933 | 8.690 | 18.644 | 1.00 | 11.53 |
| ATOM | 644 | O | ASN | A | 614 | 13.298 | 7.647 | 18.493 | 1.00 | 9.32 |
| ATOM | 645 | N | TRP | A | 615 | 13.342 | 9.850 | 18.890 | 1.00 | 8.95 |
| ATOM | 646 | CA | TRP | A | 615 | 11.893 | 9.929 | 19.015 | 1.00 | 9.02 |
| ATOM | 647 | CB | TRP | A | 615 | 11.457 | 11.397 | 19.127 | 1.00 | 9.00 |
| ATOM | 648 | CG | TRP | A | 615 | 10.002 | 11.638 | 18.809 | 1.00 | 8.75 |
| ATOM | 649 | CD2 | TRP | A | 615 | 8.860 | 11.228 | 19.581 | 1.00 | 10.58 |
| ATOM | 650 | CE2 | TRP | A | 615 | 7.709 | 11.655 | 18.883 | 1.00 | 12.68 |
| ATOM | 651 | CE3 | TRP | A | 615 | 8.698 | 10.542 | 20.789 | 1.00 | 12.70 |
| ATOM | 652 | CD1 | TRP | A | 615 | 9.504 | 12.279 | 17.709 | 1.00 | 11.21 |
| ATOM | 653 | NE1 | TRP | A | 615 | 8.132 | 12.292 | 17.747 | 1.00 | 11.25 |
| ATOM | 654 | CZ2 | TRP | A | 615 | 6.414 | 11.418 | 19.354 | 1.00 | 12.28 |
| ATOM | 655 | CZ3 | TRP | A | 615 | 7.410 | 10.307 | 21.255 | 1.00 | 18.15 |
| ATOM | 656 | CH2 | TRP | A | 615 | 6.286 | 10.744 | 20.536 | 1.00 | 13.05 |
| ATOM | 657 | C | TRP | A | 615 | 11.090 | 9.237 | 17.910 | 1.00 | 8.08 |
| ATOM | 658 | O | TRP | A | 615 | 10.093 | 8.579 | 18.197 | 1.00 | 9.24 |
| ATOM | 659 | N | ARG | A | 616 | 11.514 | 9.365 | 16.656 | 1.00 | 9.61 |
| ATOM | 660 | CA | ARG | A | 616 | 10.750 | 8.759 | 15.570 | 1.00 | 11.24 |
| ATOM | 661 | CB | ARG | A | 616 | 11.346 | 9.129 | 14.206 | 1.00 | 9.33 |
| ATOM | 662 | CG | ARG | A | 616 | 11.241 | 10.630 | 13.885 | 1.00 | 13.33 |
| ATOM | 663 | CD | ARG | A | 616 | 9.817 | 11.169 | 14.120 | 1.00 | 11.22 |
| ATOM | 664 | NE | ARG | A | 616 | 9.705 | 12.598 | 13.823 | 1.00 | 13.61 |
| ATOM | 665 | CZ | ARG | A | 616 | 9.119 | 13.103 | 12.741 | 1.00 | 15.86 |
| ATOM | 666 | NH1 | ARG | A | 616 | 8.577 | 12.300 | 11.834 | 1.00 | 15.89 |
| ATOM | 667 | NH2 | ARG | A | 616 | 9.073 | 14.419 | 12.564 | 1.00 | 19.53 |
| ATOM | 668 | C | ARG | A | 616 | 10.607 | 7.250 | 15.705 | 1.00 | 9.88 |
| ATOM | 669 | O | ARG | A | 616 | 9.575 | 6.692 | 15.328 | 1.00 | 10.40 |
| ATOM | 670 | N | HIS | A | 617 | 11.625 | 6.582 | 16.242 | 1.00 | 7.87 |
| ATOM | 671 | CA | HIS | A | 617 | 11.514 | 5.141 | 16.419 | 1.00 | 7.37 |
| ATOM | 672 | CB | HIS | A | 617 | 12.862 | 4.523 | 16.782 | 1.00 | 11.98 |
| ATOM | 673 | CG | HIS | A | 617 | 12.742 | 3.163 | 17.390 | 1.00 | 8.70 |
| ATOM | 674 | CD2 | HIS | A | 617 | 13.043 | 2.715 | 18.632 | 1.00 | 9.51 |
| ATOM | 675 | ND1 | HIS | A | 617 | 12.206 | 2.088 | 16.713 | 1.00 | 8.99 |
| ATOM | 676 | CE1 | HIS | A | 617 | 12.183 | 1.036 | 17.510 | 1.00 | 11.83 |
| ATOM | 677 | NE2 | HIS | A | 617 | 12.684 | 1.391 | 18.681 | 1.00 | 10.58 |
| ATOM | 678 | C | HIS | A | 617 | 10.488 | 4.816 | 17.506 | 1.00 | 8.63 |
| ATOM | 679 | O | HIS | A | 617 | 9.692 | 3.888 | 17.361 | 1.00 | 10.24 |
| ATOM | 680 | N | ALA | A | 618 | 10.498 | 5.586 | 18.591 | 1.00 | 7.45 |
| ATOM | 681 | CA | ALA | A | 618 | 9.549 | 5.358 | 19.687 | 1.00 | 8.54 |
| ATOM | 682 | CB | ALA | A | 618 | 9.887 | 6.261 | 20.864 | 1.00 | 11.31 |
| ATOM | 683 | C | ALA | A | 618 | 8.126 | 5.637 | 19.210 | 1.00 | 8.39 |
| ATOM | 684 | O | ALA | A | 618 | 7.184 | 4.900 | 19.523 | 1.00 | 9.03 |
| ATOM | 685 | N | PHE | A | 619 | 7.981 | 6.725 | 18.465 | 1.00 | 8.88 |
| ATOM | 686 | CA | PHE | A | 619 | 6.695 | 7.118 | 17.911 | 1.00 | 9.63 |
| ATOM | 687 | CB | PHE | A | 619 | 6.850 | 8.418 | 17.119 | 1.00 | 12.92 |
| ATOM | 688 | CG | PHE | A | 619 | 5.618 | 8.809 | 16.357 | 1.00 | 10.44 |
| ATOM | 689 | CD1 | PHE | A | 619 | 4.432 | 9.066 | 17.021 | 1.00 | 18.21 |
| ATOM | 690 | CD2 | PHE | A | 619 | 5.646 | 8.914 | 14.974 | 1.00 | 12.41 |
| ATOM | 691 | CE1 | PHE | A | 619 | 3.291 | 9.412 | 16.324 | 1.00 | 14.74 |
| ATOM | 692 | CE2 | PHE | A | 619 | 4.508 | 9.260 | 14.270 | 1.00 | 13.16 |
| ATOM | 693 | CZ | PHE | A | 619 | 3.328 | 9.512 | 14.947 | 1.00 | 11.01 |
| ATOM | 694 | C | PHE | A | 619 | 6.150 | 6.017 | 16.996 | 1.00 | 9.52 |
| ATOM | 695 | O | PHE | A | 619 | 4.975 | 5.654 | 17.079 | 1.00 | 9.30 |
| ATOM | 696 | N | ASN | A | 620 | 7.001 | 5.490 | 16.119 | 1.00 | 10.15 |
| ATOM | 697 | CA | ASN | A | 620 | 6.583 | 4.428 | 15.204 | 1.00 | 11.34 |
| ATOM | 698 | CB | ASN | A | 620 | 7.694 | 4.123 | 14.194 | 1.00 | 9.10 |
| ATOM | 699 | CG | ASN | A | 620 | 7.658 | 5.052 | 12.999 | 1.00 | 17.16 |
| ATOM | 700 | OD1 | ASN | A | 620 | 8.665 | 5.656 | 12.634 | 1.00 | 19.51 |
| ATOM | 701 | ND2 | ASN | A | 620 | 6.490 | 5.166 | 12.382 | 1.00 | 12.88 |
| ATOM | 702 | C | ASN | A | 620 | 6.206 | 3.161 | 15.956 | 1.00 | 9.23 |
| ATOM | 703 | O | ASN | A | 620 | 5.273 | 2.457 | 15.574 | 1.00 | 10.76 |
| ATOM | 704 | N | THR | A | 621 | 6.936 | 2.871 | 17.026 | 1.00 | 8.56 |
| ATOM | 705 | CA | THR | A | 621 | 6.650 | 1.688 | 17.819 | 1.00 | 9.52 |
| ATOM | 706 | CB | THR | A | 621 | 7.707 | 1.509 | 18.928 | 1.00 | 8.96 |
| ATOM | 707 | OG1 | THR | A | 621 | 9.010 | 1.404 | 18.328 | 1.00 | 9.14 |
| ATOM | 708 | CG2 | THR | A | 621 | 7.428 | 0.244 | 19.736 | 1.00 | 7.93 |
| ATOM | 709 | C | THR | A | 621 | 5.252 | 1.821 | 18.425 | 1.00 | 10.48 |
| ATOM | 710 | O | THR | A | 621 | 4.455 | 0.879 | 18.382 | 1.00 | 10.11 |
| ATOM | 711 | N | ALA | A | 622 | 4.954 | 2.995 | 18.978 | 1.00 | 8.32 |
| ATOM | 712 | CA | ALA | A | 622 | 3.644 | 3.244 | 19.573 | 1.00 | 9.28 |
| ATOM | 713 | CB | ALA | A | 622 | 3.642 | 4.583 | 20.294 | 1.00 | 9.90 |
| ATOM | 714 | C | ALA | A | 622 | 2.567 | 3.224 | 18.488 | 1.00 | 10.02 |
| ATOM | 715 | O | ALA | A | 622 | 1.447 | 2.769 | 18.721 | 1.00 | 8.06 |
| ATOM | 716 | N | GLN | A | 623 | 2.902 | 3.708 | 17.297 | 1.00 | 7.82 |
| ATOM | 717 | CA | GLN | A | 623 | 1.927 | 3.708 | 16.211 | 1.00 | 10.17 |
| ATOM | 718 | CB | GLN | A | 623 | 2.456 | 4.487 | 15.003 | 1.00 | 8.39 |
| ATOM | 719 | CG | GLN | A | 623 | 1.473 | 4.578 | 13.825 | 1.00 | 8.23 |
| ATOM | 720 | CD | GLN | A | 623 | 1.444 | 3.319 | 12.967 | 1.00 | 13.63 |
| ATOM | 721 | OE1 | GLN | A | 623 | 2.484 | 2.730 | 12.682 | 1.00 | 12.75 |
| ATOM | 722 | NE2 | GLN | A | 623 | 0.258 | 2.919 | 12.535 | 1.00 | 11.69 |
| ATOM | 723 | C | GLN | A | 623 | 1.597 | 2.278 | 15.806 | 1.00 | 10.35 |
| ATOM | 724 | O | GLN | A | 623 | 0.449 | 1.962 | 15.501 | 1.00 | 9.61 |
| ATOM | 725 | N | CYS | A | 624 | 2.601 | 1.406 | 15.802 | 1.00 | 9.08 |
| ATOM | 726 | CA | CYS | A | 624 | 2.355 | 0.017 | 15.439 | 1.00 | 9.34 |
| ATOM | 727 | CB | CYS | A | 624 | 3.679 | −0.740 | 15.273 | 1.00 | 11.00 |
| ATOM | 728 | SG | CYS | A | 624 | 3.481 | −2.446 | 14.713 | 1.00 | 11.71 |
| ATOM | 729 | C | CYS | A | 624 | 1.495 | −0.625 | 16.529 | 1.00 | 10.17 |
| ATOM | 730 | O | CYS | A | 624 | 0.631 | −1.448 | 16.239 | 1.00 | 10.64 |
| ATOM | 731 | N | MET | A | 625 | 1.728 | −0.238 | 17.781 | 1.00 | 9.06 |
| ATOM | 732 | CA | MET | A | 625 | 0.933 | −0.764 | 18.892 | 1.00 | 9.38 |
| ATOM | 733 | CB | MET | A | 625 | 1.418 | −0.180 | 20.225 | 1.00 | 8.21 |
| ATOM | 734 | CG | MET | A | 625 | 0.692 | −0.718 | 21.466 | 1.00 | 8.16 |
| ATOM | 735 | SD | MET | A | 625 | 0.904 | −2.512 | 21.728 | 1.00 | 11.88 |
| ATOM | 736 | CE | MET | A | 625 | 2.600 | −2.586 | 22.309 | 1.00 | 11.24 |
| ATOM | 737 | C | MET | A | 625 | −0.529 | −0.376 | 18.661 | 1.00 | 9.31 |
| ATOM | 738 | O | MET | A | 625 | −1.434 | −1.208 | 18.757 | 1.00 | 10.06 |
| ATOM | 739 | N | PHE | A | 626 | −0.757 | 0.896 | 18.353 | 1.00 | 9.47 |
| ATOM | 740 | CA | PHE | A | 626 | −2.112 | 1.377 | 18.092 | 1.00 | 9.60 |
| ATOM | 741 | CB | PHE | A | 626 | −2.077 | 2.859 | 17.725 | 1.00 | 9.70 |
| ATOM | 742 | CG | PHE | A | 626 | −3.432 | 3.451 | 17.461 | 1.00 | 7.25 |
| ATOM | 743 | CD1 | PHE | A | 626 | −4.241 | 3.858 | 18.509 | 1.00 | 13.86 |
| ATOM | 744 | CD2 | PHE | A | 626 | −3.897 | 3.598 | 16.163 | 1.00 | 13.47 |
| ATOM | 745 | CE1 | PHE | A | 626 | −5.491 | 4.411 | 18.267 | 1.00 | 18.88 |
| ATOM | 746 | CE2 | PHE | A | 626 | −5.144 | 4.147 | 15.917 | 1.00 | 11.81 |
| ATOM | 747 | CZ | PHE | A | 626 | −5.942 | 4.552 | 16.970 | 1.00 | 16.53 |
| ATOM | 748 | C | PHE | A | 626 | −2.737 | 0.579 | 16.940 | 1.00 | 10.98 |
| ATOM | 749 | O | PHE | A | 626 | −3.862 | 0.084 | 17.047 | 1.00 | 12.71 |
| ATOM | 750 | N | ALA | A | 627 | −1.999 | 0.455 | 15.838 | 1.00 | 7.24 |
| ATOM | 751 | CA | ALA | A | 627 | −2.486 | −0.279 | 14.675 | 1.00 | 9.31 |
| ATOM | 752 | CB | ALA | A | 627 | −1.454 | −0.220 | 13.549 | 1.00 | 14.42 |
| ATOM | 753 | C | ALA | A | 627 | −2.803 | −1.734 | 15.024 | 1.00 | 11.79 |
| ATOM | 754 | O | ALA | A | 627 | −3.815 | −2.276 | 14.583 | 1.00 | 11.59 |
| ATOM | 755 | N | ALA | A | 628 | −1.945 | −2.361 | 15.824 | 1.00 | 10.63 |
| ATOM | 756 | CA | ALA | A | 628 | −2.158 | −3.749 | 16.219 | 1.00 | 11.00 |
| ATOM | 757 | CB | ALA | A | 628 | −0.934 | −4.280 | 16.961 | 1.00 | 11.43 |
| ATOM | 758 | C | ALA | A | 628 | −3.398 | −3.877 | 17.099 | 1.00 | 11.16 |
| ATOM | 759 | O | ALA | A | 628 | −4.152 | −4.845 | 16.994 | 1.00 | 12.30 |
| ATOM | 760 | N | LEU | A | 629 | −3.608 | −2.896 | 17.970 | 1.00 | 9.63 |
| ATOM | 761 | CA | LEU | A | 629 | −4.765 | −2.924 | 18.856 | 1.00 | 9.96 |
| ATOM | 762 | CB | LEU | A | 629 | −4.627 | −1.858 | 19.945 | 1.00 | 12.79 |
| ATOM | 763 | CG | LEU | A | 629 | −3.538 | −2.115 | 20.990 | 1.00 | 10.87 |
| ATOM | 764 | CD1 | LEU | A | 629 | −3.286 | −0.859 | 21.792 | 1.00 | 11.38 |
| ATOM | 765 | CD2 | LEU | A | 629 | −3.956 | −3.264 | 21.897 | 1.00 | 14.99 |
| ATOM | 766 | C | LEU | A | 629 | −6.051 | −2.692 | 18.073 | 1.00 | 12.31 |
| ATOM | 767 | O | LEU | A | 629 | −7.084 | −3.300 | 18.364 | 1.00 | 11.63 |
| ATOM | 768 | N | LYS | A | 630 | −5.973 | −1.822 | 17.070 | 1.00 | 11.64 |
| ATOM | 769 | CA | LYS | A | 630 | −7.125 | −1.479 | 16.242 | 1.00 | 12.87 |
| ATOM | 770 | CB | LYS | A | 630 | −6.984 | −0.037 | 15.738 | 1.00 | 15.53 |
| ATOM | 771 | CG | LYS | A | 630 | −7.084 | 1.030 | 16.820 | 1.00 | 13.30 |
| ATOM | 772 | CD | LYS | A | 630 | −8.495 | 1.104 | 17.385 | 1.00 | 19.60 |
| ATOM | 773 | CE | LYS | A | 630 | −8.698 | 2.349 | 18.245 | 1.00 | 18.93 |
| ATOM | 774 | NZ | LYS | A | 630 | −7.877 | 2.334 | 19.481 | 1.00 | 40.94 |
| ATOM | 775 | C | LYS | A | 630 | −7.323 | −2.422 | 15.049 | 1.00 | 13.54 |
| ATOM | 776 | O | LYS | A | 630 | −8.122 | −3.350 | 15.107 | 1.00 | 12.81 |
| ATOM | 777 | N | ALA | A | 631 | −6.597 | −2.170 | 13.967 | 1.00 | 13.05 |
| ATOM | 778 | CA | ALA | A | 631 | −6.701 | −2.997 | 12.770 | 1.00 | 14.94 |
| ATOM | 779 | CB | ALA | A | 631 | −5.763 | −2.460 | 11.688 | 1.00 | 17.58 |
| ATOM | 780 | C | ALA | A | 631 | −6.374 | −4.461 | 13.068 | 1.00 | 16.22 |
| ATOM | 781 | O | ALA | A | 631 | −6.960 | −5.375 | 12.479 | 1.00 | 15.44 |
| ATOM | 782 | N | GLY | A | 632 | −5.439 | −4.670 | 13.990 | 1.00 | 12.88 |
| ATOM | 783 | CA | GLY | A | 632 | −5.028 | −6.014 | 14.359 | 1.00 | 13.10 |
| ATOM | 784 | C | GLY | A | 632 | −5.995 | −6.711 | 15.298 | 1.00 | 13.12 |
| ATOM | 785 | O | GLY | A | 632 | −5.850 | −7.902 | 15.576 | 1.00 | 15.03 |
| ATOM | 786 | N | LYS | A | 633 | −6.968 | −5.959 | 15.803 | 1.00 | 13.75 |
| ATOM | 787 | CA | LYS | A | 633 | −7.997 | −6.497 | 16.692 | 1.00 | 14.20 |
| ATOM | 788 | CB | LYS | A | 633 | −8.815 | −7.566 | 15.954 | 1.00 | 13.72 |
| ATOM | 789 | CG | LYS | A | 633 | −9.438 | −7.109 | 14.631 | 1.00 | 23.64 |
| ATOM | 790 | CD | LYS | A | 633 | −10.532 | −6.072 | 14.834 | 1.00 | 30.20 |
| ATOM | 791 | CE | LYS | A | 633 | −11.182 | −5.672 | 13.510 | 1.00 | 34.87 |
| ATOM | 792 | NZ | LYS | A | 633 | −10.227 | −4.989 | 12.594 | 1.00 | 46.21 |
| ATOM | 793 | C | LYS | A | 633 | −7.464 | −7.085 | 17.997 | 1.00 | 14.18 |
| ATOM | 794 | O | LYS | A | 633 | −8.048 | −8.024 | 18.540 | 1.00 | 14.72 |
| ATOM | 795 | N | ILE | A | 634 | −6.366 | −6.536 | 18.507 | 1.00 | 13.07 |
| ATOM | 796 | CA | ILE | A | 634 | −5.801 | −7.037 | 19.755 | 1.00 | 12.44 |
| ATOM | 797 | CB | ILE | A | 634 | −4.262 | −6.819 | 19.799 | 1.00 | 13.52 |
| ATOM | 798 | CG2 | ILE | A | 634 | −3.709 | −7.236 | 21.156 | 1.00 | 14.73 |
| ATOM | 799 | CG1 | ILE | A | 634 | −3.581 | −7.632 | 18.694 | 1.00 | 14.10 |
| ATOM | 800 | CD1 | ILE | A | 634 | −3.757 | −9.128 | 18.829 | 1.00 | 14.44 |
| ATOM | 801 | C | ILE | A | 634 | −6.439 | −6.346 | 20.967 | 1.00 | 12.23 |
| ATOM | 802 | O | ILE | A | 634 | −6.456 | −6.894 | 22.067 | 1.00 | 12.06 |
| ATOM | 803 | N | GLN | A | 635 | −6.990 | −5.155 | 20.752 | 1.00 | 13.17 |
| ATOM | 804 | CA | GLN | A | 635 | −7.597 | −4.377 | 21.834 | 1.00 | 12.59 |
| ATOM | 805 | CB | GLN | A | 635 | −8.330 | −3.158 | 21.267 | 1.00 | 14.77 |
| ATOM | 806 | CG | GLN | A | 635 | −8.796 | −2.180 | 22.345 | 1.00 | 17.07 |
| ATOM | 807 | CD | GLN | A | 635 | −9.479 | −0.949 | 21.778 | 1.00 | 17.87 |
| ATOM | 808 | OE1 | GLN | A | 635 | −9.080 | −0.425 | 20.741 | 1.00 | 15.93 |
| ATOM | 809 | NE2 | GLN | A | 635 | −10.509 | −0.473 | 22.471 | 1.00 | 27.59 |
| ATOM | 810 | C | GLN | A | 635 | −8.549 | −5.127 | 22.762 | 1.00 | 13.46 |
| ATOM | 811 | O | GLN | A | 635 | −8.382 | −5.107 | 23.984 | 1.00 | 12.66 |
| ATOM | 812 | N | ASN | A | 636 | −9.554 | −5.778 | 22.188 | 1.00 | 12.36 |
| ATOM | 813 | CA | ASN | A | 636 | −10.533 | −6.493 | 22.996 | 1.00 | 15.16 |
| ATOM | 814 | CB | ASN | A | 636 | −11.736 | −6.872 | 22.130 | 1.00 | 13.54 |
| ATOM | 815 | CG | ASN | A | 636 | −12.508 | −5.655 | 21.658 | 1.00 | 19.96 |
| ATOM | 816 | OD1 | ASN | A | 636 | −12.211 | −4.526 | 22.059 | 1.00 | 19.43 |
| ATOM | 817 | ND2 | ASN | A | 636 | −13.506 | −5.874 | 20.810 | 1.00 | 20.88 |
| ATOM | 818 | C | ASN | A | 636 | −10.002 | −7.708 | 23.743 | 1.00 | 14.39 |
| ATOM | 819 | O | ASN | A | 636 | −10.720 | −8.312 | 24.539 | 1.00 | 17.61 |
| ATOM | 820 | N | LYS | A | 637 | −8.744 | −8.060 | 23.500 | 1.00 | 13.69 |
| ATOM | 821 | CA | LYS | A | 637 | −8.132 | −9.194 | 24.180 | 1.00 | 13.94 |
| ATOM | 822 | CB | LYS | A | 637 | −7.124 | −9.893 | 23.262 | 1.00 | 13.19 |
| ATOM | 823 | CG | LYS | A | 637 | −7.667 | −10.328 | 21.910 | 1.00 | 16.41 |
| ATOM | 824 | CD | LYS | A | 637 | −6.599 | −11.079 | 21.128 | 1.00 | 18.79 |
| ATOM | 825 | CE | LYS | A | 637 | −7.110 | −11.542 | 19.773 | 1.00 | 21.00 |
| ATOM | 826 | NZ | LYS | A | 637 | −8.266 | −12.467 | 19.893 | 1.00 | 27.72 |
| ATOM | 827 | C | LYS | A | 637 | −7.400 | −8.708 | 25.435 | 1.00 | 14.29 |
| ATOM | 828 | O | LYS | A | 637 | −6.930 | −9.514 | 26.237 | 1.00 | 15.91 |
| ATOM | 829 | N | LEU | A | 638 | −7.316 | −7.391 | 25.611 | 1.00 | 13.88 |
| ATOM | 830 | CA | LEU | A | 638 | −6.602 | −6.828 | 26.757 | 1.00 | 14.02 |
| ATOM | 831 | CB | LEU | A | 638 | −5.426 | −5.988 | 26.256 | 1.00 | 14.42 |
| ATOM | 832 | CG | LEU | A | 638 | −4.496 | −6.595 | 25.201 | 1.00 | 13.65 |
| ATOM | 833 | CD1 | LEU | A | 638 | −3.405 | −5.589 | 24.860 | 1.00 | 13.98 |
| ATOM | 834 | CD2 | LEU | A | 638 | −3.881 | −7.885 | 25.718 | 1.00 | 12.70 |
| ATOM | 835 | C | LEU | A | 638 | −7.464 | −5.973 | 27.687 | 1.00 | 13.83 |
| ATOM | 836 | O | LEU | A | 638 | −8.551 | −5.529 | 27.313 | 1.00 | 16.24 |
| ATOM | 837 | N | THR | A | 639 | −6.968 | −5.745 | 28.902 | 1.00 | 13.04 |
| ATOM | 838 | CA | THR | A | 639 | −7.665 | −4.913 | 29.885 | 1.00 | 12.97 |
| ATOM | 839 | CB | THR | A | 639 | −7.225 | −5.244 | 31.323 | 1.00 | 9.72 |
| ATOM | 840 | OG1 | THR | A | 639 | −5.883 | −4.777 | 31.534 | 1.00 | 11.58 |
| ATOM | 841 | CG2 | THR | A | 639 | −7.284 | −6.752 | 31.574 | 1.00 | 13.01 |
| ATOM | 842 | C | THR | A | 639 | −7.320 | −3.443 | 29.627 | 1.00 | 11.14 |
| ATOM | 843 | O | THR | A | 639 | −6.369 | −3.147 | 28.910 | 1.00 | 12.39 |
| ATOM | 844 | N | ASP | A | 640 | −8.082 | −2.524 | 30.216 | 1.00 | 11.83 |
| ATOM | 845 | CA | ASP | A | 640 | −7.814 | −1.098 | 30.027 | 1.00 | 11.03 |
| ATOM | 846 | CB | ASP | A | 640 | −8.846 | −0.243 | 30.774 | 1.00 | 9.58 |
| ATOM | 847 | CG | ASP | A | 640 | −10.238 | −0.368 | 30.191 | 1.00 | 23.27 |
| ATOM | 848 | OD1 | ASP | A | 640 | −10.357 | −0.828 | 29.037 | 1.00 | 21.74 |
| ATOM | 849 | OD2 | ASP | A | 640 | −11.211 | 0.011 | 30.879 | 1.00 | 18.96 |
| ATOM | 850 | C | ASP | A | 640 | −6.419 | −0.724 | 30.519 | 1.00 | 10.85 |
| ATOM | 851 | O | ASP | A | 640 | −5.708 | 0.058 | 29.881 | 1.00 | 11.05 |
| ATOM | 852 | N | LEU | A | 641 | −6.031 | −1.290 | 31.655 | 1.00 | 11.16 |
| ATOM | 853 | CA | LEU | A | 641 | −4.727 | −1.004 | 32.235 | 1.00 | 10.51 |
| ATOM | 854 | CB | LEU | A | 641 | −4.628 | −1.606 | 33.640 | 1.00 | 13.05 |
| ATOM | 855 | CG | LEU | A | 641 | −5.633 | −1.081 | 34.674 | 1.00 | 13.28 |
| ATOM | 856 | CD1 | LEU | A | 641 | −5.383 | −1.762 | 36.005 | 1.00 | 14.91 |
| ATOM | 857 | CD2 | LEU | A | 641 | −5.506 | 0.432 | 34.813 | 1.00 | 12.98 |
| ATOM | 858 | C | LEU | A | 641 | −3.593 | −1.527 | 31.359 | 1.00 | 10.02 |
| ATOM | 859 | O | LEU | A | 641 | −2.550 | −0.883 | 31.237 | 1.00 | 9.79 |
| ATOM | 860 | N | GLU | A | 642 | −3.791 | −2.692 | 30.748 | 1.00 | 9.49 |
| ATOM | 861 | CA | GLU | A | 642 | −2.756 | −3.253 | 29.886 | 1.00 | 9.55 |
| ATOM | 862 | CB | GLU | A | 642 | −3.120 | −4.687 | 29.493 | 1.00 | 13.24 |
| ATOM | 863 | CG | GLU | A | 642 | −3.215 | −5.605 | 30.705 | 1.00 | 17.56 |
| ATOM | 864 | CD | GLU | A | 642 | −3.645 | −7.021 | 30.373 | 1.00 | 14.47 |
| ATOM | 865 | OE1 | GLU | A | 642 | −4.411 | −7.212 | 29.404 | 1.00 | 12.63 |
| ATOM | 866 | OE2 | GLU | A | 642 | −3.233 | −7.949 | 31.108 | 1.00 | 15.36 |
| ATOM | 867 | C | GLU | A | 642 | −2.568 | −2.366 | 28.656 | 1.00 | 9.56 |
| ATOM | 868 | O | GLU | A | 642 | −1.442 | −2.135 | 28.217 | 1.00 | 10.36 |
| ATOM | 869 | N | ILE | A | 643 | −3.665 | −1.847 | 28.112 | 1.00 | 9.98 |
| ATOM | 870 | CA | ILE | A | 643 | −3.585 | −0.978 | 26.945 | 1.00 | 11.53 |
| ATOM | 871 | CB | ILE | A | 643 | −4.992 | −0.683 | 26.391 | 1.00 | 12.52 |
| ATOM | 872 | CG2 | ILE | A | 643 | −4.915 | 0.367 | 25.281 | 1.00 | 11.83 |
| ATOM | 873 | CG1 | ILE | A | 643 | −5.614 | −1.984 | 25.885 | 1.00 | 11.82 |
| ATOM | 874 | CD1 | ILE | A | 643 | −7.089 | −1.872 | 25.553 | 1.00 | 18.49 |
| ATOM | 875 | C | ILE | A | 643 | −2.883 | 0.323 | 27.339 | 1.00 | 9.56 |
| ATOM | 876 | O | ILE | A | 643 | −1.993 | 0.806 | 26.635 | 1.00 | 10.76 |
| ATOM | 877 | N | LEU | A | 644 | −3.278 | 0.880 | 28.479 | 1.00 | 9.68 |
| ATOM | 878 | CA | LEU | A | 644 | −2.663 | 2.105 | 28.974 | 1.00 | 9.78 |
| ATOM | 879 | CB | LEU | A | 644 | −3.285 | 2.482 | 30.321 | 1.00 | 11.15 |
| ATOM | 880 | CG | LEU | A | 644 | −2.658 | 3.643 | 31.093 | 1.00 | 14.02 |
| ATOM | 881 | CD1 | LEU | A | 644 | −2.853 | 4.946 | 30.328 | 1.00 | 14.15 |
| ATOM | 882 | CD2 | LEU | A | 644 | −3.315 | 3.735 | 32.471 | 1.00 | 14.10 |
| ATOM | 883 | C | LEU | A | 644 | −1.152 | 1.899 | 29.139 | 1.00 | 9.98 |
| ATOM | 884 | O | LEU | A | 644 | −0.342 | 2.720 | 28.694 | 1.00 | 9.97 |
| ATOM | 885 | N | ALA | A | 645 | −0.778 | 0.788 | 29.766 | 1.00 | 8.98 |
| ATOM | 886 | CA | ALA | A | 645 | 0.634 | 0.492 | 30.008 | 1.00 | 8.84 |
| ATOM | 887 | CB | ALA | A | 645 | 0.763 | −0.724 | 30.928 | 1.00 | 8.17 |
| ATOM | 888 | C | ALA | A | 645 | 1.414 | 0.256 | 28.718 | 1.00 | 9.39 |
| ATOM | 889 | O | ALA | A | 645 | 2.556 | 0.687 | 28.598 | 1.00 | 9.92 |
| ATOM | 890 | N | LEU | A | 646 | 0.799 | −0.430 | 27.761 | 1.00 | 8.76 |
| ATOM | 891 | CA | LEU | A | 646 | 1.462 | −0.708 | 26.488 | 1.00 | 8.34 |
| ATOM | 892 | CB | LEU | A | 646 | 0.607 | −1.652 | 25.644 | 1.00 | 10.30 |
| ATOM | 893 | CG | LEU | A | 646 | 0.541 | −3.101 | 26.128 | 1.00 | 11.47 |
| ATOM | 894 | CD1 | LEU | A | 646 | −0.557 | −3.832 | 25.379 | 1.00 | 15.28 |
| ATOM | 895 | CD2 | LEU | A | 646 | 1.879 | −3.779 | 25.906 | 1.00 | 10.48 |
| ATOM | 896 | C | LEU | A | 646 | 1.761 | 0.550 | 25.682 | 1.00 | 7.55 |
| ATOM | 897 | O | LEU | A | 646 | 2.837 | 0.676 | 25.094 | 1.00 | 9.05 |
| ATOM | 898 | N | LEU | A | 647 | 0.812 | 1.479 | 25.643 | 1.00 | 7.57 |
| ATOM | 899 | CA | LEU | A | 647 | 1.020 | 2.713 | 24.889 | 1.00 | 8.87 |
| ATOM | 900 | CB | LEU | A | 647 | −0.283 | 3.516 | 24.803 | 1.00 | 7.82 |
| ATOM | 901 | CG | LEU | A | 647 | −0.214 | 4.722 | 23.862 | 1.00 | 10.06 |
| ATOM | 902 | CD1 | LEU | A | 647 | 0.194 | 4.251 | 22.470 | 1.00 | 13.81 |
| ATOM | 903 | CD2 | LEU | A | 647 | −1.560 | 5.437 | 23.820 | 1.00 | 12.05 |
| ATOM | 904 | C | LEU | A | 647 | 2.120 | 3.550 | 25.540 | 1.00 | 8.12 |
| ATOM | 905 | O | LEU | A | 647 | 3.011 | 4.070 | 24.863 | 1.00 | 8.78 |
| ATOM | 906 | N | ILE | A | 648 | 2.056 | 3.676 | 26.860 | 1.00 | 9.66 |
| ATOM | 907 | CA | ILE | A | 648 | 3.065 | 4.429 | 27.597 | 1.00 | 8.86 |
| ATOM | 908 | CB | ILE | A | 648 | 2.692 | 4.501 | 29.100 | 1.00 | 10.81 |
| ATOM | 909 | CG2 | ILE | A | 648 | 3.851 | 5.063 | 29.917 | 1.00 | 7.70 |
| ATOM | 910 | CG1 | ILE | A | 648 | 1.439 | 5.364 | 29.266 | 1.00 | 11.45 |
| ATOM | 911 | CD1 | ILE | A | 648 | 0.921 | 5.437 | 30.688 | 1.00 | 9.57 |
| ATOM | 912 | C | ILE | A | 648 | 4.427 | 3.756 | 27.424 | 1.00 | 8.38 |
| ATOM | 913 | O | ILE | A | 648 | 5.439 | 4.427 | 27.180 | 1.00 | 9.63 |
| ATOM | 914 | N | ALA | A | 649 | 4.456 | 2.430 | 27.536 | 1.00 | 9.32 |
| ATOM | 915 | CA | ALA | A | 649 | 5.710 | 1.697 | 27.388 | 1.00 | 9.98 |
| ATOM | 916 | CB | ALA | A | 649 | 5.506 | 0.214 | 27.703 | 1.00 | 11.64 |
| ATOM | 917 | C | ALA | A | 649 | 6.285 | 1.853 | 25.989 | 1.00 | 9.64 |
| ATOM | 918 | O | ALA | A | 649 | 7.471 | 2.126 | 25.831 | 1.00 | 10.34 |
| ATOM | 919 | N | ALA | A | 650 | 5.449 | 1.680 | 24.971 | 1.00 | 11.02 |
| ATOM | 920 | CA | ALA | A | 650 | 5.914 | 1.814 | 23.591 | 1.00 | 9.43 |
| ATOM | 921 | CB | ALA | A | 650 | 4.747 | 1.618 | 22.617 | 1.00 | 8.79 |
| ATOM | 922 | C | ALA | A | 650 | 6.561 | 3.183 | 23.359 | 1.00 | 8.11 |
| ATOM | 923 | O | ALA | A | 650 | 7.628 | 3.292 | 22.748 | 1.00 | 9.60 |
| ATOM | 924 | N | LEU | A | 651 | 5.914 | 4.233 | 23.851 | 1.00 | 9.58 |
| ATOM | 925 | CA | LEU | A | 651 | 6.443 | 5.582 | 23.679 | 1.00 | 10.27 |
| ATOM | 926 | CB | LEU | A | 651 | 5.383 | 6.613 | 24.066 | 1.00 | 9.84 |
| ATOM | 927 | CG | LEU | A | 651 | 4.198 | 6.717 | 23.101 | 1.00 | 6.24 |
| ATOM | 928 | CD1 | LEU | A | 651 | 3.038 | 7.432 | 23.772 | 1.00 | 14.57 |
| ATOM | 929 | CD2 | LEU | A | 651 | 4.637 | 7.463 | 21.846 | 1.00 | 11.43 |
| ATOM | 930 | C | LEU | A | 651 | 7.706 | 5.848 | 24.488 | 1.00 | 10.46 |
| ATOM | 931 | O | LEU | A | 651 | 8.573 | 6.611 | 24.066 | 1.00 | 9.75 |
| ATOM | 932 | N | SER | A | 652 | 7.807 | 5.194 | 25.640 | 1.00 | 9.58 |
| ATOM | 933 | CA | SER | A | 652 | 8.935 | 5.394 | 26.542 | 1.00 | 10.30 |
| ATOM | 934 | CB | SER | A | 652 | 8.422 | 5.453 | 27.983 | 1.00 | 9.30 |
| ATOM | 935 | OG | SER | A | 652 | 7.367 | 6.379 | 28.133 | 1.00 | 11.78 |
| ATOM | 936 | C | SER | A | 652 | 10.026 | 4.334 | 26.504 | 1.00 | 8.64 |
| ATOM | 937 | O | SER | A | 652 | 11.068 | 4.520 | 27.125 | 1.00 | 9.90 |
| ATOM | 938 | N | HIS | A | 653 | 9.805 | 3.249 | 25.765 | 1.00 | 9.64 |
| ATOM | 939 | CA | HIS | A | 653 | 10.739 | 2.128 | 25.769 | 1.00 | 9.99 |
| ATOM | 940 | CB | HIS | A | 653 | 10.164 | 0.963 | 24.952 | 1.00 | 8.26 |
| ATOM | 941 | CG | HIS | A | 653 | 10.546 | 0.980 | 23.510 | 1.00 | 8.55 |
| ATOM | 942 | CD2 | HIS | A | 653 | 11.529 | 0.333 | 22.842 | 1.00 | 9.53 |
| ATOM | 943 | ND1 | HIS | A | 653 | 9.881 | 1.740 | 22.569 | 1.00 | 10.01 |
| ATOM | 944 | CE1 | HIS | A | 653 | 10.440 | 1.559 | 21.387 | 1.00 | 6.46 |
| ATOM | 945 | NE2 | HIS | A | 653 | 11.444 | 0.708 | 21.526 | 1.00 | 6.65 |
| ATOM | 946 | C | HIS | A | 653 | 12.210 | 2.321 | 25.412 | 1.00 | 10.62 |
| ATOM | 947 | O | HIS | A | 653 | 13.024 | 1.459 | 25.741 | 1.00 | 8.86 |
| ATOM | 948 | N | ASP | A | 654 | 12.559 | 3.424 | 24.752 | 1.00 | 9.82 |
| ATOM | 949 | CA | ASP | A | 654 | 13.959 | 3.673 | 24.406 | 1.00 | 9.46 |
| ATOM | 950 | CB | ASP | A | 654 | 14.184 | 3.580 | 22.889 | 1.00 | 10.10 |
| ATOM | 951 | CG | ASP | A | 654 | 14.509 | 2.172 | 22.432 | 1.00 | 10.77 |
| ATOM | 952 | OD1 | ASP | A | 654 | 15.159 | 1.444 | 23.204 | 1.00 | 9.55 |
| ATOM | 953 | OD2 | ASP | A | 654 | 14.141 | 1.812 | 21.294 | 1.00 | 8.85 |
| ATOM | 954 | C | ASP | A | 654 | 14.477 | 5.024 | 24.896 | 1.00 | 9.92 |
| ATOM | 955 | O | ASP | A | 654 | 15.500 | 5.507 | 24.413 | 1.00 | 9.67 |
| ATOM | 956 | N | LEU | A | 655 | 13.783 | 5.619 | 25.865 | 1.00 | 9.19 |
| ATOM | 957 | CA | LEU | A | 655 | 14.169 | 6.922 | 26.408 | 1.00 | 10.06 |
| ATOM | 958 | CB | LEU | A | 655 | 13.364 | 7.214 | 27.677 | 1.00 | 8.55 |
| ATOM | 959 | CG | LEU | A | 655 | 11.900 | 7.603 | 27.479 | 1.00 | 10.24 |
| ATOM | 960 | CD1 | LEU | A | 655 | 11.151 | 7.468 | 28.805 | 1.00 | 9.10 |
| ATOM | 961 | CD2 | LEU | A | 655 | 11.819 | 9.028 | 26.944 | 1.00 | 15.71 |
| ATOM | 962 | C | LEU | A | 655 | 15.658 | 7.101 | 26.713 | 1.00 | 10.50 |
| ATOM | 963 | O | LEU | A | 655 | 16.279 | 6.268 | 27.372 | 1.00 | 9.92 |
| ATOM | 964 | N | ASP | A | 656 | 16.215 | 8.209 | 26.232 | 1.00 | 8.66 |
| ATOM | 965 | CA | ASP | A | 656 | 17.622 | 8.542 | 26.448 | 1.00 | 8.66 |
| ATOM | 966 | CB | ASP | A | 656 | 17.867 | 8.811 | 27.941 | 1.00 | 11.08 |
| ATOM | 967 | CG | ASP | A | 656 | 19.211 | 9.475 | 28.207 | 1.00 | 16.07 |
| ATOM | 968 | OD1 | ASP | A | 656 | 19.576 | 10.406 | 27.460 | 1.00 | 11.04 |
| ATOM | 969 | OD2 | ASP | A | 656 | 19.897 | 9.074 | 29.173 | 1.00 | 13.88 |
| ATOM | 970 | C | ASP | A | 656 | 18.600 | 7.483 | 25.939 | 1.00 | 8.59 |
| ATOM | 971 | O | ASP | A | 656 | 19.670 | 7.288 | 26.511 | 1.00 | 9.89 |
| ATOM | 972 | N | HIS | A | 657 | 18.242 | 6.806 | 24.851 | 1.00 | 9.07 |
| ATOM | 973 | CA | HIS | A | 657 | 19.118 | 5.790 | 24.284 | 1.00 | 10.63 |
| ATOM | 974 | CB | HIS | A | 657 | 18.412 | 5.068 | 23.137 | 1.00 | 9.06 |
| ATOM | 975 | CG | HIS | A | 657 | 19.095 | 3.810 | 22.712 | 1.00 | 8.47 |
| ATOM | 976 | CD2 | HIS | A | 657 | 20.347 | 3.589 | 22.245 | 1.00 | 7.97 |
| ATOM | 977 | ND1 | HIS | A | 657 | 18.480 | 2.575 | 22.748 | 1.00 | 12.46 |
| ATOM | 978 | CE1 | HIS | A | 657 | 19.322 | 1.652 | 22.322 | 1.00 | 5.38 |
| ATOM | 979 | NE2 | HIS | A | 657 | 20.464 | 2.242 | 22.011 | 1.00 | 12.33 |
| ATOM | 980 | C | HIS | A | 657 | 20.404 | 6.453 | 23.778 | 1.00 | 11.40 |
| ATOM | 981 | O | HIS | A | 657 | 20.362 | 7.404 | 22.993 | 1.00 | 11.45 |
| ATOM | 982 | N | PRO | A | 658 | 21.567 | 5.957 | 24.230 | 1.00 | 11.01 |
| ATOM | 983 | CD | PRO | A | 658 | 21.697 | 4.963 | 25.311 | 1.00 | 10.94 |
| ATOM | 984 | CA | PRO | A | 658 | 22.884 | 6.479 | 23.850 | 1.00 | 10.19 |
| ATOM | 985 | CB | PRO | A | 658 | 23.792 | 5.930 | 24.944 | 1.00 | 9.66 |
| ATOM | 986 | CG | PRO | A | 658 | 23.171 | 4.618 | 25.252 | 1.00 | 16.61 |
| ATOM | 987 | C | PRO | A | 658 | 23.386 | 6.123 | 22.451 | 1.00 | 11.02 |
| ATOM | 988 | O | PRO | A | 658 | 24.428 | 6.627 | 22.021 | 1.00 | 12.41 |
| ATOM | 989 | N | GLY | A | 659 | 22.659 | 5.265 | 21.744 | 1.00 | 8.82 |
| ATOM | 990 | CA | GLY | A | 659 | 23.080 | 4.888 | 20.405 | 1.00 | 8.78 |
| ATOM | 991 | C | GLY | A | 659 | 24.115 | 3.781 | 20.356 | 1.00 | 9.97 |
| ATOM | 992 | O | GLY | A | 659 | 24.750 | 3.566 | 19.316 | 1.00 | 12.32 |
| ATOM | 993 | N | VAL | A | 660 | 24.305 | 3.095 | 21.480 | 1.00 | 8.93 |
| ATOM | 994 | CA | VAL | A | 660 | 25.248 | 1.983 | 21.564 | 1.00 | 9.69 |
| ATOM | 995 | CB | VAL | A | 660 | 26.554 | 2.387 | 22.301 | 1.00 | 10.19 |
| ATOM | 996 | CG1 | VAL | A | 660 | 27.358 | 3.355 | 21.434 | 1.00 | 12.07 |
| ATOM | 997 | CG2 | VAL | A | 660 | 26.236 | 3.020 | 23.642 | 1.00 | 12.93 |
| ATOM | 998 | C | VAL | A | 660 | 24.545 | 0.834 | 22.289 | 1.00 | 10.76 |
| ATOM | 999 | O | VAL | A | 660 | 23.601 | 1.059 | 23.052 | 1.00 | 10.23 |
| ATOM | 1000 | N | SER | A | 661 | 25.002 | −0.392 | 22.049 | 1.00 | 7.63 |
| ATOM | 1001 | CA | SER | A | 661 | 24.371 | −1.577 | 22.624 | 1.00 | 10.33 |
| ATOM | 1002 | CB | SER | A | 661 | 24.748 | −2.801 | 21.793 | 1.00 | 8.76 |
| ATOM | 1003 | OG | SER | A | 661 | 26.099 | −3.151 | 22.021 | 1.00 | 9.56 |
| ATOM | 1004 | C | SER | A | 661 | 24.687 | −1.864 | 24.088 | 1.00 | 9.36 |
| ATOM | 1005 | O | SER | A | 661 | 25.549 | −1.230 | 24.696 | 1.00 | 10.42 |
| ATOM | 1006 | N | ASN | A | 662 | 23.969 | −2.838 | 24.643 | 1.00 | 10.10 |
| ATOM | 1007 | CA | ASN | A | 662 | 24.165 | −3.261 | 26.026 | 1.00 | 8.94 |
| ATOM | 1008 | CB | ASN | A | 662 | 23.177 | −4.369 | 26.384 | 1.00 | 12.24 |
| ATOM | 1009 | CG | ASN | A | 662 | 21.832 | −3.836 | 26.824 | 1.00 | 11.27 |
| ATOM | 1010 | OD1 | ASN | A | 662 | 20.794 | −4.457 | 26.592 | 1.00 | 19.20 |
| ATOM | 1011 | ND2 | ASN | A | 662 | 21.844 | −2.689 | 27.483 | 1.00 | 5.90 |
| ATOM | 1012 | C | ASN | A | 662 | 25.586 | −3.781 | 26.161 | 1.00 | 11.89 |
| ATOM | 1013 | O | ASN | A | 662 | 26.289 | −3.459 | 27.116 | 1.00 | 12.39 |
| ATOM | 1014 | N | GLN | A | 663 | 26.010 | −4.598 | 25.199 | 1.00 | 10.34 |
| ATOM | 1015 | CA | GLN | A | 663 | 27.356 | −5.139 | 25.246 | 1.00 | 11.06 |
| ATOM | 1016 | CB | GLN | A | 663 | 27.578 | −6.120 | 24.083 | 1.00 | 14.83 |
| ATOM | 1017 | CG | GLN | A | 663 | 26.677 | −7.373 | 24.197 | 1.00 | 25.28 |
| ATOM | 1018 | CD | GLN | A | 663 | 25.283 | −7.191 | 23.575 | 1.00 | 24.96 |
| ATOM | 1019 | OE1 | GLN | A | 663 | 24.919 | −6.113 | 23.106 | 1.00 | 31.10 |
| ATOM | 1020 | NE2 | GLN | A | 663 | 24.497 | −8.271 | 23.572 | 1.00 | 46.85 |
| ATOM | 1021 | C | GLN | A | 663 | 28.422 | −4.039 | 25.236 | 1.00 | 13.51 |
| ATOM | 1022 | O | GLN | A | 663 | 29.439 | −4.161 | 25.921 | 1.00 | 12.70 |
| ATOM | 1023 | N | PHE | A | 664 | 28.184 | −2.956 | 24.504 | 1.00 | 12.64 |
| ATOM | 1024 | CA | PHE | A | 664 | 29.145 | −1.852 | 24.465 | 1.00 | 11.20 |
| ATOM | 1025 | CB | PHE | A | 664 | 28.754 | −0.838 | 23.380 | 1.00 | 12.60 |
| ATOM | 1026 | CG | PHE | A | 664 | 29.669 | 0.355 | 23.296 | 1.00 | 15.21 |
| ATOM | 1027 | CD1 | PHE | A | 664 | 29.554 | 1.407 | 24.198 | 1.00 | 13.93 |
| ATOM | 1028 | CD2 | PHE | A | 664 | 30.668 | 0.409 | 22.336 | 1.00 | 10.34 |
| ATOM | 1029 | CE1 | PHE | A | 664 | 30.419 | 2.484 | 24.144 | 1.00 | 11.59 |
| ATOM | 1030 | CE2 | PHE | A | 664 | 31.541 | 1.487 | 22.275 | 1.00 | 16.42 |
| ATOM | 1031 | CZ | PHE | A | 664 | 31.418 | 2.527 | 23.182 | 1.00 | 18.27 |
| ATOM | 1032 | C | PHE | A | 664 | 29.207 | −1.171 | 25.830 | 1.00 | 11.75 |
| ATOM | 1033 | O | PHE | A | 664 | 30.290 | −0.818 | 26.306 | 1.00 | 11.28 |
| ATOM | 1034 | N | LEU | A | 665 | 28.046 | −0.980 | 26.452 | 1.00 | 11.17 |
| ATOM | 1035 | CA | LEU | A | 665 | 27.982 | −0.346 | 27.768 | 1.00 | 11.46 |
| ATOM | 1036 | CB | LEU | A | 665 | 26.524 | −0.163 | 28.204 | 1.00 | 11.38 |
| ATOM | 1037 | CG | LEU | A | 665 | 25.703 | 0.864 | 27.420 | 1.00 | 9.72 |
| ATOM | 1038 | CD1 | LEU | A | 665 | 24.304 | 0.959 | 28.015 | 1.00 | 16.35 |
| ATOM | 1039 | CD2 | LEU | A | 665 | 26.399 | 2.215 | 27.470 | 1.00 | 13.26 |
| ATOM | 1040 | C | LEU | A | 665 | 28.726 | −1.200 | 28.793 | 1.00 | 13.75 |
| ATOM | 1041 | O | LEU | A | 665 | 29.404 | −0.686 | 29.681 | 1.00 | 11.85 |
| ATOM | 1042 | N | ILE | A | 666 | 28.591 | −2.512 | 28.661 | 1.00 | 12.41 |
| ATOM | 1043 | CA | ILE | A | 666 | 29.259 | −3.444 | 29.559 | 1.00 | 13.52 |
| ATOM | 1044 | CB | ILE | A | 666 | 28.773 | −4.887 | 29.300 | 1.00 | 13.10 |
| ATOM | 1045 | CG2 | ILE | A | 666 | 29.667 | −5.882 | 30.020 | 1.00 | 13.00 |
| ATOM | 1046 | CG1 | ILE | A | 666 | 27.322 | −5.036 | 29.761 | 1.00 | 10.73 |
| ATOM | 1047 | CD1 | ILE | A | 666 | 26.687 | −6.346 | 29.369 | 1.00 | 14.67 |
| ATOM | 1048 | C | ILE | A | 666 | 30.770 | −3.381 | 29.356 | 1.00 | 11.71 |
| ATOM | 1049 | O | ILE | A | 666 | 31.541 | −3.297 | 30.317 | 1.00 | 14.40 |
| ATOM | 1050 | N | ASN | A | 667 | 31.183 | −3.408 | 28.094 | 1.00 | 12.52 |
| ATOM | 1051 | CA | ASN | A | 667 | 32.599 | −3.382 | 27.733 | 1.00 | 11.69 |
| ATOM | 1052 | CB | ASN | A | 667 | 32.745 | −3.652 | 26.232 | 1.00 | 15.94 |
| ATOM | 1053 | CG | ASN | A | 667 | 32.340 | −5.065 | 25.848 | 1.00 | 13.91 |
| ATOM | 1054 | OD1 | ASN | A | 667 | 32.138 | −5.363 | 24.669 | 1.00 | 17.63 |
| ATOM | 1055 | ND2 | ASN | A | 667 | 32.233 | −5.946 | 26.837 | 1.00 | 11.25 |
| ATOM | 1056 | C | ASN | A | 667 | 33.323 | −2.090 | 28.091 | 1.00 | 15.59 |
| ATOM | 1057 | O | ASN | A | 667 | 34.530 | −2.104 | 28.355 | 1.00 | 16.72 |
| ATOM | 1058 | N | THR | A | 668 | 32.601 | −0.974 | 28.098 | 1.00 | 15.05 |
| ATOM | 1059 | CA | THR | A | 668 | 33.211 | 0.308 | 28.428 | 1.00 | 16.29 |
| ATOM | 1060 | CB | THR | A | 668 | 32.670 | 1.432 | 27.520 | 1.00 | 16.12 |
| ATOM | 1061 | OG1 | THR | A | 668 | 31.237 | 1.448 | 27.567 | 1.00 | 19.49 |
| ATOM | 1062 | CG2 | THR | A | 668 | 33.133 | 1.207 | 26.078 | 1.00 | 15.93 |
| ATOM | 1063 | C | THR | A | 668 | 32.995 | 0.672 | 29.895 | 1.00 | 18.10 |
| ATOM | 1064 | O | THR | A | 668 | 33.158 | 1.830 | 30.297 | 1.00 | 20.65 |
| ATOM | 1065 | N | ASN | A | 669 | 32.617 | −0.330 | 30.682 | 1.00 | 17.75 |
| ATOM | 1066 | CA | ASN | A | 669 | 32.402 | −0.177 | 32.115 | 1.00 | 20.15 |
| ATOM | 1067 | CB | ASN | A | 669 | 33.749 | 0.053 | 32.797 | 1.00 | 28.92 |
| ATOM | 1068 | CG | ASN | A | 669 | 34.791 | −0.955 | 32.361 | 1.00 | 34.79 |
| ATOM | 1069 | OD1 | ASN | A | 669 | 34.619 | −2.160 | 32.543 | 1.00 | 40.75 |
| ATOM | 1070 | ND2 | ASN | A | 669 | 35.877 | −0.468 | 31.772 | 1.00 | 36.76 |
| ATOM | 1071 | C | ASN | A | 669 | 31.428 | 0.926 | 32.515 | 1.00 | 20.87 |
| ATOM | 1072 | O | ASN | A | 669 | 31.690 | 1.696 | 33.443 | 1.00 | 21.59 |
| ATOM | 1073 | N | SER | A | 670 | 30.302 | 0.996 | 31.819 | 1.00 | 17.34 |
| ATOM | 1074 | CA | SER | A | 670 | 29.286 | 1.992 | 32.120 | 1.00 | 19.60 |
| ATOM | 1075 | CB | SER | A | 670 | 28.103 | 1.836 | 31.160 | 1.00 | 18.96 |
| ATOM | 1076 | OG | SER | A | 670 | 27.023 | 2.672 | 31.535 | 1.00 | 22.69 |
| ATOM | 1077 | C | SER | A | 670 | 28.803 | 1.787 | 33.551 | 1.00 | 16.24 |
| ATOM | 1078 | O | SER | A | 670 | 28.755 | 0.655 | 34.041 | 1.00 | 14.46 |
| ATOM | 1079 | N | GLU | A | 671 | 28.455 | 2.879 | 34.227 | 1.00 | 18.45 |
| ATOM | 1080 | CA | GLU | A | 671 | 27.951 | 2.775 | 35.589 | 1.00 | 19.69 |
| ATOM | 1081 | CB | GLU | A | 671 | 27.756 | 4.154 | 36.209 | 1.00 | 17.48 |
| ATOM | 1082 | CG | GLU | A | 671 | 28.985 | 5.024 | 36.189 | 1.00 | 31.14 |
| ATOM | 1083 | CD | GLU | A | 671 | 28.901 | 6.156 | 37.191 | 1.00 | 32.78 |
| ATOM | 1084 | OE1 | GLU | A | 671 | 27.830 | 6.796 | 37.291 | 1.00 | 31.62 |
| ATOM | 1085 | OE2 | GLU | A | 671 | 29.914 | 6.406 | 37.875 | 1.00 | 26.52 |
| ATOM | 1086 | C | GLU | A | 671 | 26.611 | 2.058 | 35.549 | 1.00 | 18.77 |
| ATOM | 1087 | O | GLU | A | 671 | 26.253 | 1.332 | 36.482 | 1.00 | 19.75 |
| ATOM | 1088 | N | LEU | A | 672 | 25.868 | 2.281 | 34.466 | 1.00 | 17.92 |
| ATOM | 1089 | CA | LEU | A | 672 | 24.569 | 1.646 | 34.279 | 1.00 | 15.76 |
| ATOM | 1090 | CB | LEU | A | 672 | 23.954 | 2.067 | 32.942 | 1.00 | 12.50 |
| ATOM | 1091 | CG | LEU | A | 672 | 23.478 | 3.509 | 32.771 | 1.00 | 18.92 |
| ATOM | 1092 | CD1 | LEU | A | 672 | 23.101 | 3.741 | 31.314 | 1.00 | 18.65 |
| ATOM | 1093 | CD2 | LEU | A | 672 | 22.292 | 3.766 | 33.678 | 1.00 | 24.52 |
| ATOM | 1094 | C | LEU | A | 672 | 24.738 | 0.130 | 34.291 | 1.00 | 13.50 |
| ATOM | 1095 | O | LEU | A | 672 | 23.961 | −0.586 | 34.912 | 1.00 | 12.92 |
| ATOM | 1096 | N | ALA | A | 673 | 25.755 | −0.358 | 33.590 | 1.00 | 13.11 |
| ATOM | 1097 | CA | ALA | A | 673 | 26.001 | −1.792 | 33.546 | 1.00 | 13.53 |
| ATOM | 1098 | CB | ALA | A | 673 | 27.169 | −2.099 | 32.594 | 1.00 | 11.10 |
| ATOM | 1099 | C | ALA | A | 673 | 26.318 | −2.283 | 34.956 | 1.00 | 13.78 |
| ATOM | 1100 | O | ALA | A | 673 | 25.925 | −3.380 | 35.354 | 1.00 | 12.36 |
| ATOM | 1101 | N | LEU | A | 674 | 27.028 | −1.458 | 35.718 | 1.00 | 15.22 |
| ATOM | 1102 | CA | LEU | A | 674 | 27.394 | −1.829 | 37.076 | 1.00 | 16.65 |
| ATOM | 1103 | CB | LEU | A | 674 | 28.414 | −0.832 | 37.632 | 1.00 | 18.96 |
| ATOM | 1104 | CG | LEU | A | 674 | 29.225 | −1.323 | 38.832 | 1.00 | 26.52 |
| ATOM | 1105 | CD1 | LEU | A | 674 | 29.939 | −2.620 | 38.460 | 1.00 | 24.45 |
| ATOM | 1106 | CD2 | LEU | A | 674 | 30.231 | −0.258 | 39.246 | 1.00 | 20.57 |
| ATOM | 1107 | C | LEU | A | 674 | 26.152 | −1.866 | 37.960 | 1.00 | 15.94 |
| ATOM | 1108 | O | LEU | A | 674 | 25.930 | −2.818 | 38.709 | 1.00 | 16.86 |
| ATOM | 1109 | N | MET | A | 675 | 25.335 | −0.822 | 37.851 | 1.00 | 19.35 |
| ATOM | 1110 | CA | MET | A | 675 | 24.107 | −0.694 | 38.627 | 1.00 | 18.57 |
| ATOM | 1111 | CB | MET | A | 675 | 23.413 | 0.641 | 38.312 | 1.00 | 29.02 |
| ATOM | 1112 | CG | MET | A | 675 | 24.180 | 1.882 | 38.753 | 1.00 | 32.86 |
| ATOM | 1113 | SD | MET | A | 675 | 23.766 | 3.373 | 37.785 | 1.00 | 38.23 |
| ATOM | 1114 | CE | MET | A | 675 | 22.129 | 3.769 | 38.381 | 1.00 | 40.96 |
| ATOM | 1115 | C | MET | A | 675 | 23.133 | −1.830 | 38.346 | 1.00 | 17.83 |
| ATOM | 1116 | O | MET | A | 675 | 22.529 | −2.383 | 39.265 | 1.00 | 17.84 |
| ATOM | 1117 | N | TYR | A | 676 | 23.000 | −2.184 | 37.073 | 1.00 | 15.75 |
| ATOM | 1118 | CA | TYR | A | 676 | 22.052 | −3.212 | 36.679 | 1.00 | 16.85 |
| ATOM | 1119 | CB | TYR | A | 676 | 21.293 | −2.720 | 35.442 | 1.00 | 16.19 |
| ATOM | 1120 | CG | TYR | A | 676 | 20.619 | −1.374 | 35.668 | 1.00 | 20.30 |
| ATOM | 1121 | CD1 | TYR | A | 676 | 19.694 | −1.197 | 36.691 | 1.00 | 31.51 |
| ATOM | 1122 | CE1 | TYR | A | 676 | 19.107 | 0.045 | 36.922 | 1.00 | 18.89 |
| ATOM | 1123 | CD2 | TYR | A | 676 | 20.934 | −0.276 | 34.877 | 1.00 | 23.78 |
| ATOM | 1124 | CE2 | TYR | A | 676 | 20.354 | 0.962 | 35.097 | 1.00 | 27.90 |
| ATOM | 1125 | CZ | TYR | A | 676 | 19.443 | 1.117 | 36.118 | 1.00 | 24.78 |
| ATOM | 1126 | OH | TYR | A | 676 | 18.866 | 2.354 | 36.326 | 1.00 | 28.03 |
| ATOM | 1127 | C | TYR | A | 676 | 22.605 | −4.623 | 36.471 | 1.00 | 16.48 |
| ATOM | 1128 | O | TYR | A | 676 | 21.949 | −5.478 | 35.872 | 1.00 | 15.88 |
| ATOM | 1129 | N | ASN | A | 677 | 23.814 | −4.863 | 36.968 | 1.00 | 16.14 |
| ATOM | 1130 | CA | ASN | A | 677 | 24.433 | −6.182 | 36.888 | 1.00 | 14.23 |
| ATOM | 1131 | CB | ASN | A | 677 | 23.666 | −7.138 | 37.812 | 1.00 | 21.33 |
| ATOM | 1132 | CG | ASN | A | 677 | 24.382 | −8.452 | 38.022 | 1.00 | 29.37 |
| ATOM | 1133 | OD1 | ASN | A | 677 | 25.570 | −8.478 | 38.349 | 1.00 | 38.44 |
| ATOM | 1134 | ND2 | ASN | A | 677 | 23.662 | −9.555 | 37.847 | 1.00 | 42.62 |
| ATOM | 1135 | C | ASN | A | 677 | 24.516 | −6.765 | 35.474 | 1.00 | 15.05 |
| ATOM | 1136 | O | ASN | A | 677 | 24.332 | −7.966 | 35.275 | 1.00 | 14.59 |
| ATOM | 1137 | N | ASP | A | 678 | 24.802 | −5.904 | 34.502 | 1.00 | 13.69 |
| ATOM | 1138 | CA | ASP | A | 678 | 24.926 | −6.302 | 33.100 | 1.00 | 16.08 |
| ATOM | 1139 | CB | ASP | A | 678 | 26.080 | −7.293 | 32.915 | 1.00 | 12.96 |
| ATOM | 1140 | CG | ASP | A | 678 | 27.430 | −6.691 | 33.234 | 1.00 | 18.27 |
| ATOM | 1141 | OD1 | ASP | A | 678 | 27.519 | −5.452 | 33.377 | 1.00 | 16.21 |
| ATOM | 1142 | OD2 | ASP | A | 678 | 28.406 | −7.464 | 33.334 | 1.00 | 20.95 |
| ATOM | 1143 | C | ASP | A | 678 | 23.667 | −6.919 | 32.499 | 1.00 | 16.35 |
| ATOM | 1144 | O | ASP | A | 678 | 23.731 | −7.509 | 31.420 | 1.00 | 17.49 |
| ATOM | 1145 | N | GLU | A | 679 | 22.534 | −6.790 | 33.184 | 1.00 | 14.03 |
| ATOM | 1146 | CA | GLU | A | 679 | 21.278 | −7.358 | 32.690 | 1.00 | 14.39 |
| ATOM | 1147 | CB | GLU | A | 679 | 20.567 | −8.133 | 33.799 | 1.00 | 14.04 |
| ATOM | 1148 | CG | GLU | A | 679 | 21.314 | −9.351 | 34.300 | 1.00 | 22.79 |
| ATOM | 1149 | CD | GLU | A | 679 | 20.514 | −10.128 | 35.330 | 1.00 | 43.42 |
| ATOM | 1150 | OE1 | GLU | A | 679 | 19.464 | −10.699 | 34.964 | 1.00 | 51.30 |
| ATOM | 1151 | OE2 | GLU | A | 679 | 20.931 | −10.162 | 36.507 | 1.00 | 41.34 |
| ATOM | 1152 | C | GLU | A | 679 | 20.320 | −6.301 | 32.151 | 1.00 | 12.89 |
| ATOM | 1153 | O | GLU | A | 679 | 19.873 | −5.430 | 32.895 | 1.00 | 13.61 |
| ATOM | 1154 | N | SER | A | 680 | 19.993 | −6.401 | 30.863 | 1.00 | 10.87 |
| ATOM | 1155 | CA | SER | A | 680 | 19.086 | −5.456 | 30.209 | 1.00 | 12.50 |
| ATOM | 1156 | CB | SER | A | 680 | 17.627 | −5.787 | 30.555 | 1.00 | 12.08 |
| ATOM | 1157 | OG | SER | A | 680 | 17.267 | −7.087 | 30.111 | 1.00 | 11.26 |
| ATOM | 1158 | C | SER | A | 680 | 19.409 | −4.038 | 30.663 | 1.00 | 11.78 |
| ATOM | 1159 | O | SER | A | 680 | 18.517 | −3.280 | 31.045 | 1.00 | 12.76 |
| ATOM | 1160 | N | VAL | A | 681 | 20.693 | −3.693 | 30.617 | 1.00 | 9.67 |
| ATOM | 1161 | CA | VAL | A | 681 | 21.183 | −2.386 | 31.049 | 1.00 | 7.19 |
| ATOM | 1162 | CB | VAL | A | 681 | 22.688 | −2.248 | 30.725 | 1.00 | 11.97 |
| ATOM | 1163 | CG1 | VAL | A | 681 | 23.219 | −0.917 | 31.245 | 1.00 | 10.98 |
| ATOM | 1164 | CG2 | VAL | A | 681 | 23.460 | −3.423 | 31.346 | 1.00 | 12.93 |
| ATOM | 1165 | C | VAL | A | 681 | 20.426 | −1.195 | 30.453 | 1.00 | 9.58 |
| ATOM | 1166 | O | VAL | A | 681 | 19.870 | −0.378 | 31.185 | 1.00 | 10.02 |
| ATOM | 1167 | N | LEU | A | 681A | 20.420 | −1.094 | 29.128 | 1.00 | 9.51 |
| ATOM | 1168 | CA | LEU | A | 681A | 19.718 | −0.011 | 28.443 | 1.00 | 10.16 |
| ATOM | 1169 | CB | LEU | A | 681A | 19.773 | −0.216 | 26.929 | 1.00 | 7.24 |
| ATOM | 1170 | CG | LEU | A | 681A | 21.079 | 0.035 | 26.178 | 1.00 | 9.74 |
| ATOM | 1171 | CD1 | LEU | A | 681A | 20.979 | −0.563 | 24.788 | 1.00 | 13.02 |
| ATOM | 1172 | CD2 | LEU | A | 681A | 21.345 | 1.526 | 26.102 | 1.00 | 15.84 |
| ATOM | 1173 | C | LEU | A | 681A | 18.253 | 0.049 | 28.851 | 1.00 | 8.34 |
| ATOM | 1174 | O | LEU | A | 681A | 17.725 | 1.118 | 29.174 | 1.00 | 10.62 |
| ATOM | 1175 | N | GLU | A | 682 | 17.599 | −1.110 | 28.834 | 1.00 | 10.08 |
| ATOM | 1176 | CA | GLU | A | 682 | 16.183 | −1.191 | 29.161 | 1.00 | 7.57 |
| ATOM | 1177 | CB | GLU | A | 682 | 15.667 | −2.599 | 28.855 | 1.00 | 6.60 |
| ATOM | 1178 | CG | GLU | A | 682 | 15.731 | −2.954 | 27.348 | 1.00 | 8.78 |
| ATOM | 1179 | CD | GLU | A | 682 | 17.151 | −3.199 | 26.835 | 1.00 | 12.12 |
| ATOM | 1180 | OE1 | GLU | A | 682 | 18.001 | −3.693 | 27.614 | 1.00 | 10.38 |
| ATOM | 1181 | OE2 | GLU | A | 682 | 17.418 | −2.919 | 25.646 | 1.00 | 10.27 |
| ATOM | 1182 | C | GLU | A | 682 | 15.867 | −0.773 | 30.594 | 1.00 | 8.81 |
| ATOM | 1183 | O | GLU | A | 682 | 14.829 | −0.158 | 30.854 | 1.00 | 7.94 |
| ATOM | 1184 | N | HIS | A | 683 | 16.752 | −1.099 | 31.526 | 1.00 | 9.05 |
| ATOM | 1185 | CA | HIS | A | 683 | 16.531 | −0.677 | 32.897 | 1.00 | 10.40 |
| ATOM | 1186 | CB | HIS | A | 683 | 17.561 | −1.324 | 33.830 | 1.00 | 11.92 |
| ATOM | 1187 | CG | HIS | A | 683 | 17.161 | −2.686 | 34.296 | 1.00 | 10.38 |
| ATOM | 1188 | CD2 | HIS | A | 683 | 17.557 | −3.920 | 33.903 | 1.00 | 9.93 |
| ATOM | 1189 | ND1 | HIS | A | 683 | 16.172 | −2.888 | 35.238 | 1.00 | 14.33 |
| ATOM | 1190 | CE1 | HIS | A | 683 | 15.977 | −4.183 | 35.400 | 1.00 | 7.54 |
| ATOM | 1191 | NE2 | HIS | A | 683 | 16.806 | −4.831 | 34.599 | 1.00 | 15.67 |
| ATOM | 1192 | C | HIS | A | 683 | 16.631 | 0.846 | 32.925 | 1.00 | 11.08 |
| ATOM | 1193 | O | HIS | A | 683 | 15.868 | 1.512 | 33.621 | 1.00 | 10.48 |
| ATOM | 1194 | N | HIS | A | 684 | 17.553 | 1.397 | 32.138 | 1.00 | 10.86 |
| ATOM | 1195 | CA | HIS | A | 684 | 17.710 | 2.844 | 32.076 | 1.00 | 9.98 |
| ATOM | 1196 | CB | HIS | A | 684 | 18.941 | 3.233 | 31.256 | 1.00 | 9.09 |
| ATOM | 1197 | CG | HIS | A | 684 | 19.229 | 4.697 | 31.284 | 1.00 | 8.54 |
| ATOM | 1198 | CD2 | HIS | A | 684 | 19.399 | 5.555 | 32.322 | 1.00 | 5.20 |
| ATOM | 1199 | ND1 | HIS | A | 684 | 19.313 | 5.467 | 30.142 | 1.00 | 17.15 |
| ATOM | 1200 | CE1 | HIS | A | 684 | 19.518 | 6.728 | 30.474 | 1.00 | 9.30 |
| ATOM | 1201 | NE2 | HIS | A | 684 | 19.573 | 6.807 | 31.795 | 1.00 | 15.79 |
| ATOM | 1202 | C | HIS | A | 684 | 16.473 | 3.502 | 31.465 | 1.00 | 9.44 |
| ATOM | 1203 | O | HIS | A | 684 | 16.020 | 4.542 | 31.941 | 1.00 | 11.75 |
| ATOM | 1204 | N | HIS | A | 685 | 15.929 | 2.898 | 30.411 | 1.00 | 9.42 |
| ATOM | 1205 | CA | HIS | A | 685 | 14.740 | 3.447 | 29.757 | 1.00 | 7.57 |
| ATOM | 1206 | CB | HIS | A | 685 | 14.361 | 2.621 | 28.521 | 1.00 | 14.86 |
| ATOM | 1207 | CG | HIS | A | 685 | 15.472 | 2.468 | 27.521 | 1.00 | 7.24 |
| ATOM | 1208 | CD2 | HIS | A | 685 | 15.846 | 1.402 | 26.773 | 1.00 | 9.32 |
| ATOM | 1209 | ND1 | HIS | A | 685 | 16.299 | 3.506 | 27.161 | 1.00 | 8.86 |
| ATOM | 1210 | CE1 | HIS | A | 685 | 17.141 | 3.088 | 26.227 | 1.00 | 14.03 |
| ATOM | 1211 | NE2 | HIS | A | 685 | 16.886 | 1.819 | 25.974 | 1.00 | 8.62 |
| ATOM | 1212 | C | HIS | A | 685 | 13.574 | 3.446 | 30.747 | 1.00 | 9.27 |
| ATOM | 1213 | O | HIS | A | 685 | 12.798 | 4.401 | 30.805 | 1.00 | 9.78 |
| ATOM | 1214 | N | PHE | A | 686 | 13.439 | 2.376 | 31.523 | 1.00 | 8.10 |
| ATOM | 1215 | CA | PHE | A | 686 | 12.351 | 2.314 | 32.507 | 1.00 | 10.05 |
| ATOM | 1216 | CB | PHE | A | 686 | 12.291 | 0.923 | 33.151 | 1.00 | 8.82 |
| ATOM | 1217 | CG | PHE | A | 686 | 11.281 | 0.808 | 34.263 | 1.00 | 10.55 |
| ATOM | 1218 | CD1 | PHE | A | 686 | 9.937 | 1.078 | 34.035 | 1.00 | 10.53 |
| ATOM | 1219 | CD2 | PHE | A | 686 | 11.674 | 0.442 | 35.538 | 1.00 | 11.42 |
| ATOM | 1220 | CE1 | PHE | A | 686 | 9.013 | 0.985 | 35.061 | 1.00 | 14.12 |
| ATOM | 1221 | CE2 | PHE | A | 686 | 10.754 | 0.348 | 36.568 | 1.00 | 15.20 |
| ATOM | 1222 | CZ | PHE | A | 686 | 9.423 | 0.618 | 36.328 | 1.00 | 13.66 |
| ATOM | 1223 | C | PHE | A | 686 | 12.554 | 3.391 | 33.570 | 1.00 | 9.59 |
| ATOM | 1224 | O | PHE | A | 686 | 11.599 | 4.036 | 34.022 | 1.00 | 11.10 |
| ATOM | 1225 | N | ASP | A | 687 | 13.807 | 3.603 | 33.965 | 1.00 | 10.38 |
| ATOM | 1226 | CA | ASP | A | 687 | 14.169 | 4.620 | 34.950 | 1.00 | 13.63 |
| ATOM | 1227 | CB | ASP | A | 687 | 15.699 | 4.646 | 35.147 | 1.00 | 15.63 |
| ATOM | 1228 | CG | ASP | A | 687 | 16.163 | 5.810 | 36.032 | 1.00 | 35.43 |
| ATOM | 1229 | OD1 | ASP | A | 687 | 15.842 | 5.812 | 37.238 | 1.00 | 39.21 |
| ATOM | 1230 | OD2 | ASP | A | 687 | 16.855 | 6.721 | 35.514 | 1.00 | 46.67 |
| ATOM | 1231 | C | ASP | A | 687 | 13.701 | 5.983 | 34.453 | 1.00 | 11.19 |
| ATOM | 1232 | O | ASP | A | 687 | 13.079 | 6.742 | 35.191 | 1.00 | 12.63 |
| ATOM | 1233 | N | GLN | A | 688 | 14.026 | 6.266 | 33.194 | 1.00 | 10.97 |
| ATOM | 1234 | CA | GLN | A | 688 | 13.664 | 7.531 | 32.571 | 1.00 | 10.87 |
| ATOM | 1235 | CB | GLN | A | 688 | 14.331 | 7.623 | 31.193 | 1.00 | 15.23 |
| ATOM | 1236 | CG | GLN | A | 688 | 15.844 | 7.876 | 31.260 | 1.00 | 11.96 |
| ATOM | 1237 | CD | GLN | A | 688 | 16.179 | 9.350 | 31.456 | 1.00 | 14.39 |
| ATOM | 1238 | OE1 | GLN | A | 688 | 15.805 | 10.193 | 30.640 | 1.00 | 20.66 |
| ATOM | 1239 | NE2 | GLN | A | 688 | 16.881 | 9.663 | 32.535 | 1.00 | 29.50 |
| ATOM | 1240 | C | GLN | A | 688 | 12.158 | 7.651 | 32.455 | 1.00 | 10.36 |
| ATOM | 1241 | O | GLN | A | 688 | 11.587 | 8.729 | 32.641 | 1.00 | 10.53 |
| ATOM | 1242 | N | CYS | A | 689 | 11.495 | 6.536 | 32.160 | 1.00 | 8.48 |
| ATOM | 1243 | CA | CYS | A | 689 | 10.043 | 6.517 | 32.040 | 1.00 | 9.59 |
| ATOM | 1244 | CB | CYS | A | 689 | 9.558 | 5.097 | 31.706 | 1.00 | 11.54 |
| ATOM | 1245 | SG | CYS | A | 689 | 7.772 | 4.940 | 31.573 | 1.00 | 11.98 |
| ATOM | 1246 | C | CYS | A | 689 | 9.406 | 7.005 | 33.343 | 1.00 | 10.54 |
| ATOM | 1247 | O | CYS | A | 689 | 8.559 | 7.905 | 33.331 | 1.00 | 10.86 |
| ATOM | 1248 | N | LEU | A | 690 | 9.822 | 6.417 | 34.463 | 1.00 | 12.43 |
| ATOM | 1249 | CA | LEU | A | 690 | 9.316 | 6.786 | 35.787 | 1.00 | 13.70 |
| ATOM | 1250 | CB | LEU | A | 690 | 9.968 | 5.912 | 36.863 | 1.00 | 15.35 |
| ATOM | 1251 | CG | LEU | A | 690 | 9.620 | 4.423 | 36.853 | 1.00 | 18.35 |
| ATOM | 1252 | CD1 | LEU | A | 690 | 10.326 | 3.723 | 38.008 | 1.00 | 25.25 |
| ATOM | 1253 | CD2 | LEU | A | 690 | 8.118 | 4.248 | 36.969 | 1.00 | 17.06 |
| ATOM | 1254 | C | LEU | A | 690 | 9.605 | 8.247 | 36.093 | 1.00 | 14.05 |
| ATOM | 1255 | O | LEU | A | 690 | 8.767 | 8.975 | 36.633 | 1.00 | 15.63 |
| ATOM | 1256 | N | MET | A | 691 | 10.817 | 8.681 | 35.750 | 1.00 | 13.87 |
| ATOM | 1257 | CA | MET | A | 691 | 11.256 | 10.042 | 35.975 | 1.00 | 14.41 |
| ATOM | 1258 | CB | MET | A | 691 | 12.676 | 10.212 | 35.411 | 1.00 | 14.62 |
| ATOM | 1259 | CG | MET | A | 691 | 13.371 | 11.496 | 35.757 | 1.00 | 32.36 |
| ATOM | 1260 | SD | MET | A | 691 | 12.902 | 12.755 | 34.635 | 1.00 | 45.55 |
| ATOM | 1261 | CE | MET | A | 691 | 13.918 | 12.471 | 33.200 | 1.00 | 37.72 |
| ATOM | 1262 | C | MET | A | 691 | 10.285 | 11.024 | 35.336 | 1.00 | 13.72 |
| ATOM | 1263 | O | MET | A | 691 | 9.863 | 11.990 | 35.971 | 1.00 | 14.45 |
| ATOM | 1264 | N | ILE | A | 692 | 9.903 | 10.758 | 34.091 | 1.00 | 10.48 |
| ATOM | 1265 | CA | ILE | A | 692 | 8.980 | 11.644 | 33.400 | 1.00 | 9.98 |
| ATOM | 1266 | CB | ILE | A | 692 | 8.966 | 11.371 | 31.895 | 1.00 | 10.00 |
| ATOM | 1267 | CG2 | ILE | A | 692 | 7.909 | 12.240 | 31.224 | 1.00 | 12.51 |
| ATOM | 1268 | CG1 | ILE | A | 692 | 10.346 | 11.691 | 31.315 | 1.00 | 14.40 |
| ATOM | 1269 | CD1 | ILE | A | 692 | 10.482 | 11.400 | 29.841 | 1.00 | 19.81 |
| ATOM | 1270 | C | ILE | A | 692 | 7.578 | 11.534 | 33.973 | 1.00 | 10.09 |
| ATOM | 1271 | O | ILE | A | 692 | 6.906 | 12.547 | 34.166 | 1.00 | 11.41 |
| ATOM | 1272 | N | LEU | A | 693 | 7.149 | 10.316 | 34.288 | 1.00 | 12.13 |
| ATOM | 1273 | CA | LEU | A | 693 | 5.821 | 10.111 | 34.862 | 1.00 | 13.85 |
| ATOM | 1274 | CB | LEU | A | 693 | 5.548 | 8.615 | 35.058 | 1.00 | 13.96 |
| ATOM | 1275 | CG | LEU | A | 693 | 5.212 | 7.826 | 33.789 | 1.00 | 10.33 |
| ATOM | 1276 | CD1 | LEU | A | 693 | 5.183 | 6.335 | 34.082 | 1.00 | 17.28 |
| ATOM | 1277 | CD2 | LEU | A | 693 | 3.868 | 8.305 | 33.243 | 1.00 | 9.40 |
| ATOM | 1278 | C | LEU | A | 693 | 5.644 | 10.837 | 36.193 | 1.00 | 14.19 |
| ATOM | 1279 | O | LEU | A | 693 | 4.524 | 11.179 | 36.577 | 1.00 | 16.64 |
| ATOM | 1280 | N | ASN | A | 694 | 6.747 | 11.073 | 36.893 | 1.00 | 16.46 |
| ATOM | 1281 | CA | ASN | A | 694 | 6.697 | 11.755 | 38.183 | 1.00 | 16.63 |
| ATOM | 1282 | CB | ASN | A | 694 | 7.686 | 11.116 | 39.159 | 1.00 | 20.12 |
| ATOM | 1283 | CG | ASN | A | 694 | 7.231 | 9.760 | 39.641 | 1.00 | 24.99 |
| ATOM | 1284 | OD1 | ASN | A | 694 | 7.869 | 8.742 | 39.373 | 1.00 | 30.33 |
| ATOM | 1285 | ND2 | ASN | A | 694 | 6.115 | 9.736 | 40.360 | 1.00 | 25.93 |
| ATOM | 1286 | C | ASN | A | 694 | 6.989 | 13.247 | 38.101 | 1.00 | 18.31 |
| ATOM | 1287 | O | ASN | A | 694 | 6.855 | 13.958 | 39.093 | 1.00 | 18.26 |
| ATOM | 1288 | N | SER | A | 695 | 7.395 | 13.721 | 36.930 | 1.00 | 16.05 |
| ATOM | 1289 | CA | SER | A | 695 | 7.704 | 15.139 | 36.755 | 1.00 | 17.70 |
| ATOM | 1290 | CB | SER | A | 695 | 8.321 | 15.376 | 35.376 | 1.00 | 19.30 |
| ATOM | 1291 | OG | SER | A | 695 | 9.607 | 14.793 | 35.298 | 1.00 | 34.04 |
| ATOM | 1292 | C | SER | A | 695 | 6.471 | 16.022 | 36.914 | 1.00 | 15.83 |
| ATOM | 1293 | O | SER | A | 695 | 5.400 | 15.710 | 36.406 | 1.00 | 17.46 |
| ATOM | 1294 | N | PRO | A | 696 | 6.611 | 17.144 | 37.630 | 1.00 | 18.97 |
| ATOM | 1295 | CD | PRO | A | 696 | 7.779 | 17.577 | 38.415 | 1.00 | 26.97 |
| ATOM | 1296 | CA | PRO | A | 696 | 5.480 | 18.053 | 37.830 | 1.00 | 18.99 |
| ATOM | 1297 | CB | PRO | A | 696 | 6.113 | 19.220 | 38.579 | 1.00 | 22.14 |
| ATOM | 1298 | CG | PRO | A | 696 | 7.154 | 18.534 | 39.414 | 1.00 | 28.71 |
| ATOM | 1299 | C | PRO | A | 696 | 4.866 | 18.480 | 36.498 | 1.00 | 16.02 |
| ATOM | 1300 | O | PRO | A | 696 | 5.585 | 18.839 | 35.564 | 1.00 | 18.08 |
| ATOM | 1301 | N | GLY | A | 697 | 3.539 | 18.425 | 36.418 | 1.00 | 16.17 |
| ATOM | 1302 | CA | GLY | A | 697 | 2.843 | 18.804 | 35.201 | 1.00 | 16.65 |
| ATOM | 1303 | C | GLY | A | 697 | 2.862 | 17.725 | 34.130 | 1.00 | 14.72 |
| ATOM | 1304 | O | GLY | A | 697 | 2.283 | 17.901 | 33.060 | 1.00 | 15.86 |
| ATOM | 1305 | N | ASN | A | 698 | 3.511 | 16.601 | 34.424 | 1.00 | 14.68 |
| ATOM | 1306 | CA | ASN | A | 698 | 3.621 | 15.493 | 33.471 | 1.00 | 14.66 |
| ATOM | 1307 | CB | ASN | A | 698 | 5.099 | 15.209 | 33.174 | 1.00 | 15.25 |
| ATOM | 1308 | CG | ASN | A | 698 | 5.760 | 16.304 | 32.365 | 1.00 | 19.94 |
| ATOM | 1309 | OD1 | ASN | A | 698 | 5.883 | 16.199 | 31.146 | 1.00 | 13.89 |
| ATOM | 1310 | ND2 | ASN | A | 698 | 6.186 | 17.367 | 33.039 | 1.00 | 16.33 |
| ATOM | 1311 | C | ASN | A | 698 | 2.990 | 14.198 | 33.973 | 1.00 | 14.82 |
| ATOM | 1312 | O | ASN | A | 698 | 3.044 | 13.174 | 33.293 | 1.00 | 13.20 |
| ATOM | 1313 | N | GLN | A | 699 | 2.379 | 14.243 | 35.151 | 1.00 | 13.46 |
| ATOM | 1314 | CA | GLN | A | 699 | 1.807 | 13.041 | 35.749 | 1.00 | 14.53 |
| ATOM | 1315 | CB | GLN | A | 699 | 1.652 | 13.281 | 37.251 | 1.00 | 16.25 |
| ATOM | 1316 | CG | GLN | A | 699 | 2.957 | 13.713 | 37.902 | 1.00 | 17.08 |
| ATOM | 1317 | CD | GLN | A | 699 | 2.787 | 14.103 | 39.350 | 1.00 | 37.15 |
| ATOM | 1318 | OE1 | GLN | A | 699 | 2.147 | 15.105 | 39.661 | 1.00 | 41.13 |
| ATOM | 1319 | NE2 | GLN | A | 699 | 3.357 | 13.307 | 40.248 | 1.00 | 41.91 |
| ATOM | 1320 | C | GLN | A | 699 | 0.499 | 12.522 | 35.158 | 1.00 | 13.96 |
| ATOM | 1321 | O | GLN | A | 699 | −0.567 | 12.676 | 35.754 | 1.00 | 15.67 |
| ATOM | 1322 | N | ILE | A | 700 | 0.582 | 11.867 | 34.003 | 1.00 | 13.44 |
| ATOM | 1323 | CA | ILE | A | 700 | −0.617 | 11.348 | 33.356 | 1.00 | 14.25 |
| ATOM | 1324 | CB | ILE | A | 700 | −0.345 | 10.974 | 31.881 | 1.00 | 15.57 |
| ATOM | 1325 | CG2 | ILE | A | 700 | 0.010 | 12.222 | 31.086 | 1.00 | 15.78 |
| ATOM | 1326 | CG1 | ILE | A | 700 | 0.777 | 9.941 | 31.793 | 1.00 | 16.74 |
| ATOM | 1327 | CD1 | ILE | A | 700 | 0.940 | 9.361 | 30.407 | 1.00 | 12.98 |
| ATOM | 1328 | C | ILE | A | 700 | −1.262 | 10.151 | 34.064 | 1.00 | 13.88 |
| ATOM | 1329 | O | ILE | A | 700 | −2.342 | 9.709 | 33.673 | 1.00 | 14.53 |
| ATOM | 1330 | N | LEU | A | 701 | −0.609 | 9.624 | 35.098 | 1.00 | 11.60 |
| ATOM | 1331 | CA | LEU | A | 701 | −1.169 | 8.495 | 35.845 | 1.00 | 14.58 |
| ATOM | 1332 | CB | LEU | A | 701 | −0.150 | 7.358 | 35.965 | 1.00 | 13.85 |
| ATOM | 1333 | CG | LEU | A | 701 | 0.310 | 6.673 | 34.677 | 1.00 | 11.07 |
| ATOM | 1334 | CD1 | LEU | A | 701 | 1.270 | 5.539 | 35.031 | 1.00 | 13.34 |
| ATOM | 1335 | CD2 | LEU | A | 701 | −0.895 | 6.128 | 33.921 | 1.00 | 12.89 |
| ATOM | 1336 | C | LEU | A | 701 | −1.612 | 8.914 | 37.247 | 1.00 | 13.75 |
| ATOM | 1337 | O | LEU | A | 701 | −2.053 | 8.084 | 38.040 | 1.00 | 15.74 |
| ATOM | 1338 | N | SER | A | 702 | −1.497 | 10.201 | 37.547 | 1.00 | 15.74 |
| ATOM | 1339 | CA | SER | A | 702 | −1.872 | 10.717 | 38.860 | 1.00 | 18.36 |
| ATOM | 1340 | CB | SER | A | 702 | −1.573 | 12.217 | 38.949 | 1.00 | 20.19 |
| ATOM | 1341 | OG | SER | A | 702 | −2.446 | 12.958 | 38.113 | 1.00 | 21.94 |
| ATOM | 1342 | C | SER | A | 702 | −3.347 | 10.490 | 39.176 | 1.00 | 19.34 |
| ATOM | 1343 | O | SER | A | 702 | −3.754 | 10.559 | 40.336 | 1.00 | 20.75 |
| ATOM | 1344 | N | GLY | A | 703 | −4.144 | 10.224 | 38.148 | 1.00 | 18.92 |
| ATOM | 1345 | CA | GLY | A | 703 | −5.565 | 10.012 | 38.359 | 1.00 | 20.40 |
| ATOM | 1346 | C | GLY | A | 703 | −5.944 | 8.600 | 38.764 | 1.00 | 21.19 |
| ATOM | 1347 | O | GLY | A | 703 | −7.036 | 8.369 | 39.284 | 1.00 | 20.43 |
| ATOM | 1348 | N | LEU | A | 704 | −5.045 | 7.649 | 38.539 | 1.00 | 16.00 |
| ATOM | 1349 | CA | LEU | A | 704 | −5.326 | 6.259 | 38.876 | 1.00 | 15.59 |
| ATOM | 1350 | CB | LEU | A | 704 | −4.262 | 5.335 | 38.278 | 1.00 | 9.29 |
| ATOM | 1351 | CG | LEU | A | 704 | −4.459 | 4.921 | 36.823 | 1.00 | 14.10 |
| ATOM | 1352 | CD1 | LEU | A | 704 | −4.510 | 6.143 | 35.926 | 1.00 | 17.73 |
| ATOM | 1353 | CD2 | LEU | A | 704 | −3.312 | 3.998 | 36.418 | 1.00 | 11.19 |
| ATOM | 1354 | C | LEU | A | 704 | −5.419 | 5.993 | 40.368 | 1.00 | 16.40 |
| ATOM | 1355 | O | LEU | A | 704 | −4.773 | 6.659 | 41.178 | 1.00 | 16.88 |
| ATOM | 1356 | N | SER | A | 705 | −6.233 | 5.004 | 40.721 | 1.00 | 15.43 |
| ATOM | 1357 | CA | SER | A | 705 | −6.395 | 4.621 | 42.111 | 1.00 | 15.96 |
| ATOM | 1358 | CB | SER | A | 705 | −7.556 | 3.640 | 42.268 | 1.00 | 15.57 |
| ATOM | 1359 | OG | SER | A | 705 | −7.242 | 2.402 | 41.658 | 1.00 | 15.44 |
| ATOM | 1360 | C | SER | A | 705 | −5.092 | 3.947 | 42.515 | 1.00 | 16.32 |
| ATOM | 1361 | O | SER | A | 705 | −4.280 | 3.583 | 41.662 | 1.00 | 17.25 |
| ATOM | 1362 | N | ILE | A | 706 | −4.886 | 3.782 | 43.812 | 1.00 | 17.56 |
| ATOM | 1363 | CA | ILE | A | 706 | −3.666 | 3.164 | 44.292 | 1.00 | 16.88 |
| ATOM | 1364 | CB | ILE | A | 706 | −3.679 | 3.086 | 45.836 | 1.00 | 21.33 |
| ATOM | 1365 | CG2 | ILE | A | 706 | −2.466 | 2.331 | 46.335 | 1.00 | 23.53 |
| ATOM | 1366 | CG1 | ILE | A | 706 | −3.712 | 4.507 | 46.411 | 1.00 | 21.96 |
| ATOM | 1367 | CD1 | ILE | A | 706 | −3.892 | 4.577 | 47.918 | 1.00 | 22.24 |
| ATOM | 1368 | C | ILE | A | 706 | −3.415 | 1.782 | 43.685 | 1.00 | 16.48 |
| ATOM | 1369 | O | ILE | A | 706 | −2.291 | 1.470 | 43.291 | 1.00 | 18.62 |
| ATOM | 1370 | N | GLU | A | 707 | −4.456 | 0.962 | 43.584 | 1.00 | 15.92 |
| ATOM | 1371 | CA | GLU | A | 707 | −4.304 | −0.376 | 43.019 | 1.00 | 14.53 |
| ATOM | 1372 | CB | GLU | A | 707 | −5.509 | −1.250 | 43.378 | 1.00 | 19.33 |
| ATOM | 1373 | CG | GLU | A | 707 | −5.518 | −1.682 | 44.836 | 1.00 | 24.82 |
| ATOM | 1374 | CD | GLU | A | 707 | −4.269 | −2.460 | 45.214 | 1.00 | 31.26 |
| ATOM | 1375 | OE1 | GLU | A | 707 | −4.029 | −3.525 | 44.610 | 1.00 | 34.47 |
| ATOM | 1376 | OE2 | GLU | A | 707 | −3.525 | −2.006 | 46.110 | 1.00 | 30.79 |
| ATOM | 1377 | C | GLU | A | 707 | −4.093 | −0.385 | 41.507 | 1.00 | 14.27 |
| ATOM | 1378 | O | GLU | A | 707 | −3.283 | −1.156 | 41.002 | 1.00 | 15.02 |
| ATOM | 1379 | N | GLU | A | 708 | −4.818 | 0.458 | 40.782 | 1.00 | 11.81 |
| ATOM | 1380 | CA | GLU | A | 708 | −4.644 | 0.495 | 39.337 | 1.00 | 12.49 |
| ATOM | 1381 | CB | GLU | A | 708 | −5.756 | 1.317 | 38.673 | 1.00 | 14.73 |
| ATOM | 1382 | CG | GLU | A | 708 | −7.134 | 0.663 | 38.813 | 1.00 | 11.98 |
| ATOM | 1383 | CD | GLU | A | 708 | −8.088 | 1.020 | 37.683 | 1.00 | 18.01 |
| ATOM | 1384 | OE1 | GLU | A | 708 | −8.121 | 2.196 | 37.274 | 1.00 | 19.76 |
| ATOM | 1385 | OE2 | GLU | A | 708 | −8.816 | 0.120 | 37.216 | 1.00 | 22.22 |
| ATOM | 1386 | C | GLU | A | 708 | −3.264 | 1.054 | 38.996 | 1.00 | 14.00 |
| ATOM | 1387 | O | GLU | A | 708 | −2.624 | 0.607 | 38.047 | 1.00 | 12.79 |
| ATOM | 1388 | N | TYR | A | 709 | −2.798 | 2.018 | 39.784 | 1.00 | 12.70 |
| ATOM | 1389 | CA | TYR | A | 709 | −1.482 | 2.604 | 39.551 | 1.00 | 13.42 |
| ATOM | 1390 | CB | TYR | A | 709 | −1.204 | 3.724 | 40.557 | 1.00 | 12.05 |
| ATOM | 1391 | CG | TYR | A | 709 | 0.129 | 4.407 | 40.339 | 1.00 | 19.13 |
| ATOM | 1392 | CD1 | TYR | A | 709 | 0.311 | 5.302 | 39.296 | 1.00 | 14.81 |
| ATOM | 1393 | CE1 | TYR | A | 709 | 1.530 | 5.928 | 39.091 | 1.00 | 15.81 |
| ATOM | 1394 | CD2 | TYR | A | 709 | 1.207 | 4.149 | 41.174 | 1.00 | 17.39 |
| ATOM | 1395 | CE2 | TYR | A | 709 | 2.427 | 4.767 | 40.978 | 1.00 | 18.97 |
| ATOM | 1396 | CZ | TYR | A | 709 | 2.583 | 5.658 | 39.934 | 1.00 | 17.67 |
| ATOM | 1397 | OH | TYR | A | 709 | 3.793 | 6.284 | 39.737 | 1.00 | 18.77 |
| ATOM | 1398 | C | TYR | A | 709 | −0.424 | 1.515 | 39.708 | 1.00 | 15.44 |
| ATOM | 1399 | O | TYR | A | 709 | 0.452 | 1.348 | 38.855 | 1.00 | 15.06 |
| ATOM | 1400 | N | LYS | A | 710 | −0.520 | 0.774 | 40.808 | 1.00 | 15.25 |
| ATOM | 1401 | CA | LYS | A | 710 | 0.409 | −0.311 | 41.095 | 1.00 | 15.31 |
| ATOM | 1402 | CB | LYS | A | 710 | −0.020 | −1.026 | 42.379 | 1.00 | 22.10 |
| ATOM | 1403 | CG | LYS | A | 710 | 0.809 | −2.249 | 42.716 | 1.00 | 23.74 |
| ATOM | 1404 | CD | LYS | A | 710 | 0.299 | −2.931 | 43.978 | 1.00 | 33.24 |
| ATOM | 1405 | CE | LYS | A | 710 | 0.996 | −4.263 | 44.204 | 1.00 | 39.08 |
| ATOM | 1406 | NZ | LYS | A | 710 | 2.478 | −4.117 | 44.248 | 1.00 | 42.37 |
| ATOM | 1407 | C | LYS | A | 710 | 0.474 | −1.309 | 39.939 | 1.00 | 14.94 |
| ATOM | 1408 | O | LYS | A | 710 | 1.555 | −1.645 | 39.448 | 1.00 | 12.87 |
| ATOM | 1409 | N | THR | A | 711 | −0.692 | −1.777 | 39.506 | 1.00 | 13.50 |
| ATOM | 1410 | CA | THR | A | 711 | −0.783 | −2.736 | 38.411 | 1.00 | 12.45 |
| ATOM | 1411 | CB | THR | A | 711 | −2.249 | −3.092 | 38.128 | 1.00 | 17.44 |
| ATOM | 1412 | OG1 | THR | A | 711 | −2.791 | −3.792 | 39.255 | 1.00 | 21.96 |
| ATOM | 1413 | CG2 | THR | A | 711 | −2.365 | −3.953 | 36.872 | 1.00 | 16.75 |
| ATOM | 1414 | C | THR | A | 711 | −0.161 | −2.198 | 37.129 | 1.00 | 11.72 |
| ATOM | 1415 | O | THR | A | 711 | 0.597 | −2.896 | 36.442 | 1.00 | 13.19 |
| ATOM | 1416 | N | THR | A | 712 | −0.487 | −0.954 | 36.809 | 1.00 | 11.15 |
| ATOM | 1417 | CA | THR | A | 712 | 0.015 | −0.319 | 35.599 | 1.00 | 13.99 |
| ATOM | 1418 | CB | THR | A | 712 | −0.598 | 1.081 | 35.424 | 1.00 | 9.97 |
| ATOM | 1419 | OG1 | THR | A | 712 | −2.029 | 0.967 | 35.357 | 1.00 | 14.40 |
| ATOM | 1420 | CG2 | THR | A | 712 | −0.088 | 1.729 | 34.133 | 1.00 | 12.92 |
| ATOM | 1421 | C | THR | A | 712 | 1.541 | −0.221 | 35.556 | 1.00 | 12.77 |
| ATOM | 1422 | O | THR | A | 712 | 2.158 | −0.596 | 34.559 | 1.00 | 12.54 |
| ATOM | 1423 | N | LEU | A | 713 | 2.156 | 0.275 | 36.625 | 1.00 | 12.50 |
| ATOM | 1424 | CA | LEU | A | 713 | 3.612 | 0.384 | 36.638 | 1.00 | 11.02 |
| ATOM | 1425 | CB | LEU | A | 713 | 4.105 | 1.023 | 37.940 | 1.00 | 13.58 |
| ATOM | 1426 | CG | LEU | A | 713 | 3.889 | 2.528 | 38.100 | 1.00 | 22.03 |
| ATOM | 1427 | CD1 | LEU | A | 713 | 4.754 | 3.042 | 39.250 | 1.00 | 21.64 |
| ATOM | 1428 | CD2 | LEU | A | 713 | 4.261 | 3.241 | 36.809 | 1.00 | 27.88 |
| ATOM | 1429 | C | LEU | A | 713 | 4.298 | −0.967 | 36.438 | 1.00 | 13.04 |
| ATOM | 1430 | O | LEU | A | 713 | 5.340 | −1.048 | 35.785 | 1.00 | 12.26 |
| ATOM | 1431 | N | LYS | A | 714 | 3.718 | −2.026 | 36.995 | 1.00 | 11.66 |
| ATOM | 1432 | CA | LYS | A | 714 | 4.304 | −3.356 | 36.852 | 1.00 | 12.24 |
| ATOM | 1433 | CB | LYS | A | 714 | 3.540 | −4.383 | 37.690 | 1.00 | 17.46 |
| ATOM | 1434 | CG | LYS | A | 714 | 3.582 | −4.123 | 39.182 | 1.00 | 27.51 |
| ATOM | 1435 | CD | LYS | A | 714 | 2.878 | −5.236 | 39.939 | 1.00 | 33.99 |
| ATOM | 1436 | CE | LYS | A | 714 | 2.899 | −4.988 | 41.433 | 1.00 | 42.93 |
| ATOM | 1437 | NZ | LYS | A | 714 | 2.222 | −6.092 | 42.168 | 1.00 | 46.33 |
| ATOM | 1438 | C | LYS | A | 714 | 4.284 | −3.784 | 35.390 | 1.00 | 12.70 |
| ATOM | 1439 | O | LYS | A | 714 | 5.259 | −4.338 | 34.886 | 1.00 | 12.91 |
| ATOM | 1440 | N | ILE | A | 715 | 3.168 | −3.525 | 34.717 | 1.00 | 10.95 |
| ATOM | 1441 | CA | ILE | A | 715 | 3.023 | −3.869 | 33.308 | 1.00 | 10.57 |
| ATOM | 1442 | CB | ILE | A | 715 | 1.586 | −3.590 | 32.820 | 1.00 | 11.39 |
| ATOM | 1443 | CG2 | ILE | A | 715 | 1.468 | −3.896 | 31.339 | 1.00 | 11.35 |
| ATOM | 1444 | CG1 | ILE | A | 715 | 0.596 | −4.450 | 33.613 | 1.00 | 12.69 |
| ATOM | 1445 | CD1 | ILE | A | 715 | −0.854 | −4.130 | 33.332 | 1.00 | 13.44 |
| ATOM | 1446 | C | ILE | A | 715 | 4.017 | −3.052 | 32.482 | 1.00 | 10.34 |
| ATOM | 1447 | O | ILE | A | 715 | 4.676 | −3.581 | 31.588 | 1.00 | 11.86 |
| ATOM | 1448 | N | ILE | A | 716 | 4.133 | −1.762 | 32.793 | 1.00 | 10.78 |
| ATOM | 1449 | CA | ILE | A | 716 | 5.069 | −0.886 | 32.081 | 1.00 | 9.90 |
| ATOM | 1450 | CB | ILE | A | 716 | 4.989 | 0.563 | 32.621 | 1.00 | 10.08 |
| ATOM | 1451 | CG2 | ILE | A | 716 | 6.171 | 1.387 | 32.115 | 1.00 | 10.86 |
| ATOM | 1452 | CG1 | ILE | A | 716 | 3.661 | 1.197 | 32.189 | 1.00 | 10.91 |
| ATOM | 1453 | CD1 | ILE | A | 716 | 3.413 | 2.564 | 32.792 | 1.00 | 11.27 |
| ATOM | 1454 | C | ILE | A | 716 | 6.508 | −1.393 | 32.196 | 1.00 | 8.77 |
| ATOM | 1455 | O | ILE | A | 716 | 7.229 | −1.487 | 31.199 | 1.00 | 9.34 |
| ATOM | 1456 | N | LYS | A | 717 | 6.926 | −1.724 | 33.413 | 1.00 | 9.18 |
| ATOM | 1457 | CA | LYS | A | 717 | 8.275 | −2.235 | 33.623 | 1.00 | 10.88 |
| ATOM | 1458 | CB | LYS | A | 717 | 8.489 | −2.606 | 35.087 | 1.00 | 12.47 |
| ATOM | 1459 | CG | LYS | A | 717 | 9.790 | −3.366 | 35.337 | 1.00 | 14.78 |
| ATOM | 1460 | CD | LYS | A | 717 | 9.865 | −3.864 | 36.775 | 1.00 | 17.70 |
| ATOM | 1461 | CE | LYS | A | 717 | 11.045 | −4.809 | 36.977 | 1.00 | 29.85 |
| ATOM | 1462 | NZ | LYS | A | 717 | 11.082 | −5.374 | 38.355 | 1.00 | 25.71 |
| ATOM | 1463 | C | LYS | A | 717 | 8.540 | −3.469 | 32.752 | 1.00 | 10.54 |
| ATOM | 1464 | O | LYS | A | 717 | 9.536 | −3.524 | 32.041 | 1.00 | 10.05 |
| ATOM | 1465 | N | GLN | A | 718 | 7.647 | −4.451 | 32.847 | 1.00 | 11.40 |
| ATOM | 1466 | CA | GLN | A | 718 | 7.774 | −5.682 | 32.067 | 1.00 | 11.80 |
| ATOM | 1467 | CB | GLN | A | 718 | 6.635 | −6.628 | 32.434 | 1.00 | 11.33 |
| ATOM | 1468 | CG | GLN | A | 718 | 6.683 | −6.965 | 33.899 | 1.00 | 14.75 |
| ATOM | 1469 | CD | GLN | A | 718 | 7.954 | −7.691 | 34.293 | 1.00 | 35.80 |
| ATOM | 1470 | OE1 | GLN | A | 718 | 8.774 | −7.176 | 35.053 | 1.00 | 49.66 |
| ATOM | 1471 | NE2 | GLN | A | 718 | 8.115 | −8.909 | 33.784 | 1.00 | 43.62 |
| ATOM | 1472 | C | GLN | A | 718 | 7.778 | −5.422 | 30.559 | 1.00 | 9.82 |
| ATOM | 1473 | O | GLN | A | 718 | 8.559 | −6.027 | 29.831 | 1.00 | 9.86 |
| ATOM | 1474 | N | ALA | A | 719 | 6.920 | −4.518 | 30.108 | 1.00 | 8.83 |
| ATOM | 1475 | CA | ALA | A | 719 | 6.842 | −4.205 | 28.686 | 1.00 | 10.49 |
| ATOM | 1476 | CB | ALA | A | 719 | 5.657 | −3.292 | 28.436 | 1.00 | 5.96 |
| ATOM | 1477 | C | ALA | A | 719 | 8.129 | −3.560 | 28.186 | 1.00 | 9.98 |
| ATOM | 1478 | O | ALA | A | 719 | 8.618 | −3.892 | 27.101 | 1.00 | 9.19 |
| ATOM | 1479 | N | ILE | A | 720 | 8.688 | −2.641 | 28.971 | 1.00 | 7.25 |
| ATOM | 1480 | CA | ILE | A | 720 | 9.920 | −1.988 | 28.548 | 1.00 | 8.66 |
| ATOM | 1481 | CB | ILE | A | 720 | 10.233 | −0.753 | 29.426 | 1.00 | 10.86 |
| ATOM | 1482 | CG2 | ILE | A | 720 | 11.616 | −0.193 | 29.086 | 1.00 | 11.64 |
| ATOM | 1483 | CG1 | ILE | A | 720 | 9.164 | 0.320 | 29.181 | 1.00 | 11.59 |
| ATOM | 1484 | CD1 | ILE | A | 720 | 9.367 | 1.605 | 29.976 | 1.00 | 12.21 |
| ATOM | 1485 | C | ILE | A | 720 | 11.085 | −2.974 | 28.580 | 1.00 | 10.02 |
| ATOM | 1486 | O | ILE | A | 720 | 11.883 | −3.040 | 27.641 | 1.00 | 8.95 |
| ATOM | 1487 | N | LEU | A | 721 | 11.178 | −3.762 | 29.643 | 1.00 | 8.10 |
| ATOM | 1488 | CA | LEU | A | 721 | 12.264 | −4.733 | 29.719 | 1.00 | 8.45 |
| ATOM | 1489 | CB | LEU | A | 721 | 12.251 | −5.446 | 31.077 | 1.00 | 10.55 |
| ATOM | 1490 | CG | LEU | A | 721 | 12.613 | −4.553 | 32.267 | 1.00 | 13.26 |
| ATOM | 1491 | CD1 | LEU | A | 721 | 12.539 | −5.350 | 33.574 | 1.00 | 15.78 |
| ATOM | 1492 | CD2 | LEU | A | 721 | 14.006 | −3.991 | 32.059 | 1.00 | 24.08 |
| ATOM | 1493 | C | LEU | A | 721 | 12.173 | −5.750 | 28.576 | 1.00 | 9.96 |
| ATOM | 1494 | O | LEU | A | 721 | 13.195 | −6.196 | 28.049 | 1.00 | 8.91 |
| ATOM | 1495 | N | ALA | A | 722 | 10.948 | −6.082 | 28.175 | 1.00 | 8.77 |
| ATOM | 1496 | CA | ALA | A | 722 | 10.715 | −7.047 | 27.100 | 1.00 | 8.74 |
| ATOM | 1497 | CB | ALA | A | 722 | 9.219 | −7.218 | 26.879 | 1.00 | 9.06 |
| ATOM | 1498 | C | ALA | A | 722 | 11.383 | −6.648 | 25.786 | 1.00 | 10.10 |
| ATOM | 1499 | O | ALA | A | 722 | 11.704 | −7.507 | 24.965 | 1.00 | 13.26 |
| ATOM | 1500 | N | THR | A | 723 | 11.598 | −5.350 | 25.585 | 1.00 | 8.97 |
| ATOM | 1501 | CA | THR | A | 723 | 12.213 | −4.884 | 24.344 | 1.00 | 10.94 |
| ATOM | 1502 | CB | THR | A | 723 | 11.943 | −3.374 | 24.127 | 1.00 | 8.53 |
| ATOM | 1503 | OG1 | THR | A | 723 | 12.542 | −2.615 | 25.181 | 1.00 | 8.41 |
| ATOM | 1504 | CG2 | THR | A | 723 | 10.437 | −3.114 | 24.118 | 1.00 | 8.22 |
| ATOM | 1505 | C | THR | A | 723 | 13.713 | −5.187 | 24.203 | 1.00 | 9.21 |
| ATOM | 1506 | O | THR | A | 723 | 14.327 | −4.862 | 23.186 | 1.00 | 12.03 |
| ATOM | 1507 | N | ASP | A | 724 | 14.308 | −5.797 | 25.223 | 1.00 | 9.75 |
| ATOM | 1508 | CA | ASP | A | 724 | 15.710 | −6.203 | 25.133 | 1.00 | 7.82 |
| ATOM | 1509 | CB | ASP | A | 724 | 16.257 | −6.533 | 26.525 | 1.00 | 12.18 |
| ATOM | 1510 | CG | ASP | A | 724 | 17.648 | −7.147 | 26.490 | 1.00 | 8.16 |
| ATOM | 1511 | OD1 | ASP | A | 724 | 18.232 | −7.299 | 25.394 | 1.00 | 11.70 |
| ATOM | 1512 | OD2 | ASP | A | 724 | 18.161 | −7.481 | 27.581 | 1.00 | 11.61 |
| ATOM | 1513 | C | ASP | A | 724 | 15.600 | −7.475 | 24.293 | 1.00 | 8.26 |
| ATOM | 1514 | O | ASP | A | 724 | 15.022 | −8.459 | 24.742 | 1.00 | 10.28 |
| ATOM | 1515 | N | LEU | A | 725 | 16.127 | −7.452 | 23.073 | 1.00 | 9.50 |
| ATOM | 1516 | CA | LEU | A | 725 | 16.037 | −8.611 | 22.189 | 1.00 | 8.83 |
| ATOM | 1517 | CB | LEU | A | 725 | 16.791 | −8.343 | 20.883 | 1.00 | 11.33 |
| ATOM | 1518 | CG | LEU | A | 725 | 16.365 | −9.236 | 19.717 | 1.00 | 16.17 |
| ATOM | 1519 | CD1 | LEU | A | 725 | 14.910 | −8.953 | 19.367 | 1.00 | 12.02 |
| ATOM | 1520 | CD2 | LEU | A | 725 | 17.262 | −8.974 | 18.516 | 1.00 | 20.09 |
| ATOM | 1521 | C | LEU | A | 725 | 16.573 | −9.880 | 22.849 | 1.00 | 12.30 |
| ATOM | 1522 | O | LEU | A | 725 | 16.179 | −10.994 | 22.498 | 1.00 | 11.52 |
| ATOM | 1523 | N | ALA | A | 726 | 17.476 | −9.712 | 23.806 | 1.00 | 11.08 |
| ATOM | 1524 | CA | ALA | A | 726 | 18.028 | −10.855 | 24.517 | 1.00 | 12.63 |
| ATOM | 1525 | CB | ALA | A | 726 | 19.104 | −10.394 | 25.489 | 1.00 | 21.41 |
| ATOM | 1526 | C | ALA | A | 726 | 16.918 | −11.583 | 25.274 | 1.00 | 12.40 |
| ATOM | 1527 | O | ALA | A | 726 | 16.914 | −12.813 | 25.354 | 1.00 | 13.56 |
| ATOM | 1528 | N | LEU | A | 727 | 15.985 | −10.823 | 25.843 | 1.00 | 12.79 |
| ATOM | 1529 | CA | LEU | A | 727 | 14.884 | −11.421 | 26.591 | 1.00 | 14.99 |
| ATOM | 1530 | CB | LEU | A | 727 | 14.206 | −10.380 | 27.485 | 1.00 | 11.71 |
| ATOM | 1531 | CG | LEU | A | 727 | 15.096 | −9.856 | 28.612 | 1.00 | 16.19 |
| ATOM | 1532 | CD1 | LEU | A | 727 | 14.302 | −8.925 | 29.511 | 1.00 | 13.74 |
| ATOM | 1533 | CD2 | LEU | A | 727 | 15.631 | −11.036 | 29.409 | 1.00 | 19.43 |
| ATOM | 1534 | C | LEU | A | 727 | 13.868 | −12.045 | 25.646 | 1.00 | 15.02 |
| ATOM | 1535 | O | LEU | A | 727 | 13.173 | −12.996 | 26.005 | 1.00 | 15.57 |
| ATOM | 1536 | N | TYR | A | 728 | 13.777 | −11.500 | 24.438 | 1.00 | 12.38 |
| ATOM | 1537 | CA | TYR | A | 728 | 12.862 | −12.038 | 23.435 | 1.00 | 12.71 |
| ATOM | 1538 | CB | TYR | A | 728 | 12.844 | −11.136 | 22.194 | 1.00 | 10.64 |
| ATOM | 1539 | CG | TYR | A | 728 | 12.303 | −11.823 | 20.960 | 1.00 | 12.57 |
| ATOM | 1540 | CD1 | TYR | A | 728 | 10.966 | −12.197 | 20.875 | 1.00 | 14.71 |
| ATOM | 1541 | CE1 | TYR | A | 728 | 10.473 | −12.854 | 19.752 | 1.00 | 12.42 |
| ATOM | 1542 | CD2 | TYR | A | 728 | 13.139 | −12.124 | 19.888 | 1.00 | 13.44 |
| ATOM | 1543 | CE2 | TYR | A | 728 | 12.659 | −12.777 | 18.763 | 1.00 | 13.13 |
| ATOM | 1544 | CZ | TYR | A | 728 | 11.329 | −13.142 | 18.699 | 1.00 | 15.27 |
| ATOM | 1545 | OH | TYR | A | 728 | 10.857 | −13.801 | 17.580 | 1.00 | 18.62 |
| ATOM | 1546 | C | TYR | A | 728 | 13.350 | −13.430 | 23.041 | 1.00 | 12.62 |
| ATOM | 1547 | O | TYR | A | 728 | 12.588 | −14.401 | 23.028 | 1.00 | 14.23 |
| ATOM | 1548 | N | ILE | A | 729 | 14.636 | −13.522 | 22.723 | 1.00 | 12.98 |
| ATOM | 1549 | CA | ILE | A | 729 | 15.232 | −14.788 | 22.327 | 1.00 | 13.19 |
| ATOM | 1550 | CB | ILE | A | 729 | 16.723 | −14.595 | 21.983 | 1.00 | 14.56 |
| ATOM | 1551 | CG2 | ILE | A | 729 | 17.395 | −15.942 | 21.797 | 1.00 | 19.39 |
| ATOM | 1552 | CG1 | ILE | A | 729 | 16.849 | −13.727 | 20.727 | 1.00 | 18.02 |
| ATOM | 1553 | CD1 | ILE | A | 729 | 18.268 | −13.309 | 20.406 | 1.00 | 26.38 |
| ATOM | 1554 | C | ILE | A | 729 | 15.088 | −15.826 | 23.439 | 1.00 | 15.70 |
| ATOM | 1555 | O | ILE | A | 729 | 14.830 | −17.001 | 23.178 | 1.00 | 14.36 |
| ATOM | 1556 | N | LYS | A | 730 | 15.227 | −15.375 | 24.681 | 1.00 | 15.21 |
| ATOM | 1557 | CA | LYS | A | 730 | 15.127 | −16.257 | 25.837 | 1.00 | 16.44 |
| ATOM | 1558 | CB | LYS | A | 730 | 15.657 | −15.528 | 27.075 | 1.00 | 21.43 |
| ATOM | 1559 | CG | LYS | A | 730 | 15.494 | −16.291 | 28.376 | 1.00 | 27.38 |
| ATOM | 1560 | CD | LYS | A | 730 | 16.133 | −15.528 | 29.523 | 1.00 | 31.22 |
| ATOM | 1561 | CE | LYS | A | 730 | 15.975 | −16.267 | 30.839 | 1.00 | 36.43 |
| ATOM | 1562 | NZ | LYS | A | 730 | 16.672 | −15.556 | 31.947 | 1.00 | 41.27 |
| ATOM | 1563 | C | LYS | A | 730 | 13.727 | −16.802 | 26.133 | 1.00 | 16.48 |
| ATOM | 1564 | O | LYS | A | 730 | 13.573 | −17.972 | 26.498 | 1.00 | 17.06 |
| ATOM | 1565 | N | ARG | A | 731 | 12.707 | −15.969 | 25.968 | 1.00 | 14.55 |
| ATOM | 1566 | CA | ARG | A | 731 | 11.347 | −16.395 | 26.283 | 1.00 | 15.57 |
| ATOM | 1567 | CB | ARG | A | 731 | 10.622 | −15.291 | 27.060 | 1.00 | 19.59 |
| ATOM | 1568 | CG | ARG | A | 731 | 11.309 | −14.842 | 28.335 | 1.00 | 18.25 |
| ATOM | 1569 | CD | ARG | A | 731 | 10.437 | −13.846 | 29.086 | 1.00 | 33.93 |
| ATOM | 1570 | NE | ARG | A | 731 | 9.209 | −14.462 | 29.588 | 1.00 | 32.78 |
| ATOM | 1571 | CZ | ARG | A | 731 | 8.168 | −13.781 | 30.058 | 1.00 | 29.98 |
| ATOM | 1572 | NH1 | ARG | A | 731 | 8.197 | −12.456 | 30.091 | 1.00 | 30.21 |
| ATOM | 1573 | NH2 | ARG | A | 731 | 7.097 | −14.426 | 30.501 | 1.00 | 36.09 |
| ATOM | 1574 | C | ARG | A | 731 | 10.461 | −16.807 | 25.118 | 1.00 | 12.75 |
| ATOM | 1575 | O | ARG | A | 731 | 9.436 | −17.454 | 25.325 | 1.00 | 15.30 |
| ATOM | 1576 | N | ARG | A | 732 | 10.841 | −16.445 | 23.897 | 1.00 | 13.21 |
| ATOM | 1577 | CA | ARG | A | 732 | 10.002 | −16.759 | 22.751 | 1.00 | 11.06 |
| ATOM | 1578 | CB | ARG | A | 732 | 10.589 | −16.162 | 21.474 | 1.00 | 10.97 |
| ATOM | 1579 | CG | ARG | A | 732 | 11.872 | −16.812 | 21.021 | 1.00 | 12.09 |
| ATOM | 1580 | CD | ARG | A | 732 | 12.325 | −16.192 | 19.724 | 1.00 | 12.79 |
| ATOM | 1581 | NE | ARG | A | 732 | 13.525 | −16.822 | 19.193 | 1.00 | 13.80 |
| ATOM | 1582 | CZ | ARG | A | 732 | 13.919 | −16.704 | 17.930 | 1.00 | 20.20 |
| ATOM | 1583 | NH1 | ARG | A | 732 | 13.201 | −15.980 | 17.077 | 1.00 | 23.11 |
| ATOM | 1584 | NH2 | ARG | A | 732 | 15.025 | −17.307 | 17.518 | 1.00 | 22.17 |
| ATOM | 1585 | C | ARG | A | 732 | 9.751 | −18.245 | 22.549 | 1.00 | 11.37 |
| ATOM | 1586 | O | ARG | A | 732 | 8.704 | −18.625 | 22.031 | 1.00 | 11.95 |
| ATOM | 1587 | N | GLY | A | 733 | 10.703 | −19.078 | 22.962 | 1.00 | 13.25 |
| ATOM | 1588 | CA | GLY | A | 733 | 10.543 | −20.512 | 22.804 | 1.00 | 13.40 |
| ATOM | 1589 | C | GLY | A | 733 | 9.298 | −21.009 | 23.508 | 1.00 | 14.10 |
| ATOM | 1590 | O | GLY | A | 733 | 8.543 | −21.823 | 22.970 | 1.00 | 15.18 |
| ATOM | 1591 | N | GLU | A | 734 | 9.080 | −20.512 | 24.719 | 1.00 | 11.89 |
| ATOM | 1592 | CA | GLU | A | 734 | 7.919 | −20.900 | 25.505 | 1.00 | 14.43 |
| ATOM | 1593 | CB | GLU | A | 734 | 7.988 | −20.264 | 26.895 | 1.00 | 14.05 |
| ATOM | 1594 | CG | GLU | A | 734 | 6.675 | −20.312 | 27.661 | 1.00 | 18.08 |
| ATOM | 1595 | CD | GLU | A | 734 | 6.810 | −19.828 | 29.091 | 1.00 | 21.97 |
| ATOM | 1596 | OE1 | GLU | A | 734 | 7.742 | −19.048 | 29.372 | 1.00 | 19.31 |
| ATOM | 1597 | OE2 | GLU | A | 734 | 5.977 | −20.221 | 29.932 | 1.00 | 32.96 |
| ATOM | 1598 | C | GLU | A | 734 | 6.632 | −20.478 | 24.806 | 1.00 | 14.65 |
| ATOM | 1599 | O | GLU | A | 734 | 5.663 | −21.235 | 24.760 | 1.00 | 14.67 |
| ATOM | 1600 | N | PHE | A | 735 | 6.631 | −19.264 | 24.267 | 1.00 | 15.01 |
| ATOM | 1601 | CA | PHE | A | 735 | 5.470 | −18.729 | 23.566 | 1.00 | 11.90 |
| ATOM | 1602 | CB | PHE | A | 735 | 5.743 | −17.278 | 23.157 | 1.00 | 11.17 |
| ATOM | 1603 | CG | PHE | A | 735 | 4.632 | −16.635 | 22.366 | 1.00 | 16.06 |
| ATOM | 1604 | CD1 | PHE | A | 735 | 3.354 | −16.515 | 22.892 | 1.00 | 16.32 |
| ATOM | 1605 | CD2 | PHE | A | 735 | 4.885 | −16.102 | 21.111 | 1.00 | 13.68 |
| ATOM | 1606 | CE1 | PHE | A | 735 | 2.355 | −15.869 | 22.178 | 1.00 | 13.80 |
| ATOM | 1607 | CE2 | PHE | A | 735 | 3.897 | −15.458 | 20.400 | 1.00 | 12.16 |
| ATOM | 1608 | CZ | PHE | A | 735 | 2.631 | −15.340 | 20.931 | 1.00 | 14.03 |
| ATOM | 1609 | C | PHE | A | 735 | 5.142 | −19.566 | 22.329 | 1.00 | 13.78 |
| ATOM | 1610 | O | PHE | A | 735 | 3.989 | −19.946 | 22.117 | 1.00 | 13.01 |
| ATOM | 1611 | N | PHE | A | 736 | 6.156 | −19.853 | 21.517 | 1.00 | 12.56 |
| ATOM | 1612 | CA | PHE | A | 736 | 5.952 | −20.632 | 20.299 | 1.00 | 14.09 |
| ATOM | 1613 | CB | PHE | A | 736 | 7.242 | −20.691 | 19.472 | 1.00 | 15.66 |
| ATOM | 1614 | CG | PHE | A | 736 | 7.752 | −19.342 | 19.031 | 1.00 | 17.77 |
| ATOM | 1615 | CD1 | PHE | A | 736 | 6.888 | −18.268 | 18.894 | 1.00 | 16.30 |
| ATOM | 1616 | CD2 | PHE | A | 736 | 9.092 | −19.166 | 18.701 | 1.00 | 14.18 |
| ATOM | 1617 | CE1 | PHE | A | 736 | 7.345 | −17.040 | 18.435 | 1.00 | 13.31 |
| ATOM | 1618 | CE2 | PHE | A | 736 | 9.558 | −17.939 | 18.242 | 1.00 | 13.12 |
| ATOM | 1619 | CZ | PHE | A | 736 | 8.686 | −16.877 | 18.106 | 1.00 | 17.60 |
| ATOM | 1620 | C | PHE | A | 736 | 5.483 | −22.054 | 20.598 | 1.00 | 14.02 |
| ATOM | 1621 | O | PHE | A | 736 | 4.653 | −22.608 | 19.874 | 1.00 | 15.27 |
| ATOM | 1622 | N | GLU | A | 737 | 6.022 | −22.640 | 21.661 | 1.00 | 15.53 |
| ATOM | 1623 | CA | GLU | A | 737 | 5.667 | −24.000 | 22.047 | 1.00 | 16.49 |
| ATOM | 1624 | CB | GLU | A | 737 | 6.598 | −24.493 | 23.155 | 1.00 | 18.16 |
| ATOM | 1625 | CG | GLU | A | 737 | 6.296 | −25.908 | 23.623 | 1.00 | 32.20 |
| ATOM | 1626 | CD | GLU | A | 737 | 7.266 | −26.397 | 24.685 | 1.00 | 44.41 |
| ATOM | 1627 | OE1 | GLU | A | 737 | 7.368 | −25.748 | 25.748 | 1.00 | 43.95 |
| ATOM | 1628 | OE2 | GLU | A | 737 | 7.926 | −27.433 | 24.457 | 1.00 | 45.00 |
| ATOM | 1629 | C | GLU | A | 737 | 4.219 | −24.105 | 22.505 | 1.00 | 16.86 |
| ATOM | 1630 | O | GLU | A | 737 | 3.517 | −25.050 | 22.137 | 1.00 | 18.64 |
| ATOM | 1631 | N | LEU | A | 738 | 3.774 | −23.144 | 23.308 | 1.00 | 15.93 |
| ATOM | 1632 | CA | LEU | A | 738 | 2.396 | −23.140 | 23.791 | 1.00 | 14.96 |
| ATOM | 1633 | CB | LEU | A | 738 | 2.137 | −21.910 | 24.665 | 1.00 | 13.51 |
| ATOM | 1634 | CG | LEU | A | 738 | 2.817 | −21.890 | 26.037 | 1.00 | 13.44 |
| ATOM | 1635 | CD1 | LEU | A | 738 | 2.813 | −20.474 | 26.589 | 1.00 | 14.08 |
| ATOM | 1636 | CD2 | LEU | A | 738 | 2.103 | −22.844 | 26.987 | 1.00 | 21.77 |
| ATOM | 1637 | C | LEU | A | 738 | 1.435 | −23.137 | 22.610 | 1.00 | 15.65 |
| ATOM | 1638 | O | LEU | A | 738 | 0.431 | −23.854 | 22.610 | 1.00 | 17.64 |
| ATOM | 1639 | N | ILE | A | 739 | 1.743 | −22.323 | 21.604 | 1.00 | 13.75 |
| ATOM | 1640 | CA | ILE | A | 739 | 0.904 | −22.236 | 20.415 | 1.00 | 14.28 |
| ATOM | 1641 | CB | ILE | A | 739 | 1.388 | −21.112 | 19.464 | 1.00 | 12.79 |
| ATOM | 1642 | CG2 | ILE | A | 739 | 0.686 | −21.224 | 18.118 | 1.00 | 23.84 |
| ATOM | 1643 | CG1 | ILE | A | 739 | 1.122 | −19.744 | 20.096 | 1.00 | 16.58 |
| ATOM | 1644 | CD1 | ILE | A | 739 | 1.656 | −18.576 | 19.279 | 1.00 | 13.81 |
| ATOM | 1645 | C | ILE | A | 739 | 0.925 | −23.557 | 19.654 | 1.00 | 16.17 |
| ATOM | 1646 | O | ILE | A | 739 | −0.126 | −24.107 | 19.309 | 1.00 | 18.24 |
| ATOM | 1647 | N | ARG | A | 740 | 2.124 | −24.075 | 19.410 | 1.00 | 15.35 |
| ATOM | 1648 | CA | ARG | A | 740 | 2.274 | −25.322 | 18.671 | 1.00 | 18.48 |
| ATOM | 1649 | CB | ARG | A | 740 | 3.758 | −25.648 | 18.474 | 1.00 | 23.15 |
| ATOM | 1650 | CG | ARG | A | 740 | 3.996 | −26.804 | 17.508 | 1.00 | 35.46 |
| ATOM | 1651 | CD | ARG | A | 740 | 5.475 | −27.059 | 17.224 | 1.00 | 39.66 |
| ATOM | 1652 | NE | ARG | A | 740 | 6.174 | −27.694 | 18.340 | 1.00 | 40.53 |
| ATOM | 1653 | CZ | ARG | A | 740 | 6.703 | −27.043 | 19.371 | 1.00 | 42.20 |
| ATOM | 1654 | NH1 | ARG | A | 740 | 6.623 | −25.721 | 19.441 | 1.00 | 52.88 |
| ATOM | 1655 | NH2 | ARG | A | 740 | 7.317 | −27.718 | 20.335 | 1.00 | 44.19 |
| ATOM | 1656 | C | ARG | A | 740 | 1.582 | −26.495 | 19.360 | 1.00 | 19.41 |
| ATOM | 1657 | O | ARG | A | 740 | 1.093 | −27.410 | 18.698 | 1.00 | 18.06 |
| ATOM | 1658 | N | LYS | A | 741 | 1.539 | −26.470 | 20.686 | 1.00 | 18.03 |
| ATOM | 1659 | CA | LYS | A | 741 | 0.906 | −27.553 | 21.430 | 1.00 | 18.11 |
| ATOM | 1660 | CB | LYS | A | 741 | 1.696 | −27.853 | 22.699 | 1.00 | 14.50 |
| ATOM | 1661 | CG | LYS | A | 741 | 3.074 | −28.426 | 22.451 | 1.00 | 21.67 |
| ATOM | 1662 | CD | LYS | A | 741 | 3.717 | −28.812 | 23.763 | 1.00 | 23.36 |
| ATOM | 1663 | CE | LYS | A | 741 | 5.087 | −29.430 | 23.558 | 1.00 | 27.38 |
| ATOM | 1664 | NZ | LYS | A | 741 | 5.715 | −29.747 | 24.871 | 1.00 | 23.91 |
| ATOM | 1665 | C | LYS | A | 741 | −0.540 | −27.248 | 21.798 | 1.00 | 17.80 |
| ATOM | 1666 | O | LYS | A | 741 | −1.134 | −27.940 | 22.623 | 1.00 | 18.63 |
| ATOM | 1667 | N | ASN | A | 742 | −1.102 | −26.214 | 21.183 | 1.00 | 17.40 |
| ATOM | 1668 | CA | ASN | A | 742 | −2.478 | −25.822 | 21.456 | 1.00 | 17.84 |
| ATOM | 1669 | CB | ASN | A | 742 | −3.443 | −26.841 | 20.849 | 1.00 | 16.88 |
| ATOM | 1670 | CG | ASN | A | 742 | −3.442 | −26.801 | 19.335 | 1.00 | 15.02 |
| ATOM | 1671 | OD1 | ASN | A | 742 | −3.741 | −25.772 | 18.733 | 1.00 | 28.97 |
| ATOM | 1672 | ND2 | ASN | A | 742 | −3.100 | −27.923 | 18.709 | 1.00 | 20.44 |
| ATOM | 1673 | C | ASN | A | 742 | −2.734 | −25.671 | 22.949 | 1.00 | 17.96 |
| ATOM | 1674 | O | ASN | A | 742 | −3.727 | −26.174 | 23.486 | 1.00 | 17.06 |
| ATOM | 1675 | N | GLN | A | 743 | −1.827 | −24.964 | 23.615 | 1.00 | 16.68 |
| ATOM | 1676 | CA | GLN | A | 743 | −1.937 | −24.727 | 25.045 | 1.00 | 17.92 |
| ATOM | 1677 | CB | GLN | A | 743 | −0.738 | −25.338 | 25.777 | 1.00 | 20.38 |
| ATOM | 1678 | CG | GLN | A | 743 | −0.461 | −26.787 | 25.408 | 1.00 | 28.60 |
| ATOM | 1679 | CD | GLN | A | 743 | 0.746 | −27.350 | 26.131 | 1.00 | 30.94 |
| ATOM | 1680 | OE1 | GLN | A | 743 | 1.775 | −26.682 | 26.255 | 1.00 | 32.52 |
| ATOM | 1681 | NE2 | GLN | A | 743 | 0.634 | −28.588 | 26.602 | 1.00 | 20.21 |
| ATOM | 1682 | C | GLN | A | 743 | −2.002 | −23.233 | 25.341 | 1.00 | 16.35 |
| ATOM | 1683 | O | GLN | A | 743 | −2.175 | −22.836 | 26.490 | 1.00 | 18.44 |
| ATOM | 1684 | N | PHE | A | 744 | −1.877 | −22.407 | 24.307 | 1.00 | 16.26 |
| ATOM | 1685 | CA | PHE | A | 744 | −1.916 | −20.959 | 24.501 | 1.00 | 19.79 |
| ATOM | 1686 | CB | PHE | A | 744 | −1.627 | −20.229 | 23.184 | 1.00 | 21.58 |
| ATOM | 1687 | CG | PHE | A | 744 | −1.465 | −18.740 | 23.340 | 1.00 | 17.97 |
| ATOM | 1688 | CD1 | PHE | A | 744 | −0.441 | −18.216 | 24.117 | 1.00 | 20.04 |
| ATOM | 1689 | CD2 | PHE | A | 744 | −2.345 | −17.865 | 22.720 | 1.00 | 24.29 |
| ATOM | 1690 | CE1 | PHE | A | 744 | −0.298 | −16.847 | 24.271 | 1.00 | 24.80 |
| ATOM | 1691 | CE2 | PHE | A | 744 | −2.208 | −16.499 | 22.869 | 1.00 | 20.38 |
| ATOM | 1692 | CZ | PHE | A | 744 | −1.184 | −15.988 | 23.647 | 1.00 | 26.24 |
| ATOM | 1693 | C | PHE | A | 744 | −3.265 | −20.515 | 25.060 | 1.00 | 19.74 |
| ATOM | 1694 | O | PHE | A | 744 | −4.316 | −20.860 | 24.526 | 1.00 | 21.03 |
| ATOM | 1695 | N | ASN | A | 745 | −3.221 | −19.746 | 26.142 | 1.00 | 19.11 |
| ATOM | 1696 | CA | ASN | A | 745 | −4.427 | −19.260 | 26.791 | 1.00 | 18.83 |
| ATOM | 1697 | CB | ASN | A | 745 | −4.893 | −20.270 | 27.839 | 1.00 | 14.11 |
| ATOM | 1698 | CG | ASN | A | 745 | −6.067 | −19.764 | 28.653 | 1.00 | 17.45 |
| ATOM | 1699 | OD1 | ASN | A | 745 | −6.924 | −19.042 | 28.143 | 1.00 | 22.19 |
| ATOM | 1700 | ND2 | ASN | A | 745 | −6.120 | −20.152 | 29.921 | 1.00 | 30.84 |
| ATOM | 1701 | C | ASN | A | 745 | −4.183 | −17.906 | 27.446 | 1.00 | 17.46 |
| ATOM | 1702 | O | ASN | A | 745 | −3.518 | −17.818 | 28.481 | 1.00 | 17.84 |
| ATOM | 1703 | N | LEU | A | 746 | −4.720 | −16.857 | 26.826 | 1.00 | 16.56 |
| ATOM | 1704 | CA | LEU | A | 746 | −4.573 | −15.497 | 27.323 | 1.00 | 18.48 |
| ATOM | 1705 | CB | LEU | A | 746 | −5.163 | −14.495 | 26.323 | 1.00 | 15.11 |
| ATOM | 1706 | CG | LEU | A | 746 | −4.319 | −14.192 | 25.082 | 1.00 | 17.98 |
| ATOM | 1707 | CD1 | LEU | A | 746 | −5.031 | −13.165 | 24.217 | 1.00 | 16.50 |
| ATOM | 1708 | CD2 | LEU | A | 746 | −2.960 | −13.664 | 25.507 | 1.00 | 17.71 |
| ATOM | 1709 | C | LEU | A | 746 | −5.212 | −15.284 | 28.688 | 1.00 | 21.08 |
| ATOM | 1710 | O | LEU | A | 746 | −4.911 | −14.304 | 29.366 | 1.00 | 20.60 |
| ATOM | 1711 | N | GLU | A | 747 | −6.098 | −16.192 | 29.089 | 1.00 | 21.14 |
| ATOM | 1712 | CA | GLU | A | 747 | −6.748 | −16.075 | 30.389 | 1.00 | 21.90 |
| ATOM | 1713 | CB | GLU | A | 747 | −7.916 | −17.058 | 30.504 | 1.00 | 20.33 |
| ATOM | 1714 | CG | GLU | A | 747 | −9.179 | −16.586 | 29.813 | 1.00 | 31.82 |
| ATOM | 1715 | CD | GLU | A | 747 | −10.324 | −17.569 | 29.959 | 1.00 | 40.12 |
| ATOM | 1716 | OE1 | GLU | A | 747 | −10.550 | −18.057 | 31.086 | 1.00 | 43.30 |
| ATOM | 1717 | OE2 | GLU | A | 747 | −11.003 | −17.846 | 28.949 | 1.00 | 40.94 |
| ATOM | 1718 | C | GLU | A | 747 | −5.740 | −16.331 | 31.498 | 1.00 | 22.98 |
| ATOM | 1719 | O | GLU | A | 747 | −5.885 | −15.826 | 32.612 | 1.00 | 23.46 |
| ATOM | 1720 | N | ASP | A | 748 | −4.719 | −17.124 | 31.188 | 1.00 | 21.44 |
| ATOM | 1721 | CA | ASP | A | 748 | −3.670 | −17.424 | 32.151 | 1.00 | 21.15 |
| ATOM | 1722 | CB | ASP | A | 748 | −2.800 | −18.579 | 31.651 | 1.00 | 19.25 |
| ATOM | 1723 | CG | ASP | A | 748 | −1.656 | −18.899 | 32.595 | 1.00 | 19.90 |
| ATOM | 1724 | OD1 | ASP | A | 748 | −0.710 | −18.088 | 32.692 | 1.00 | 26.86 |
| ATOM | 1725 | OD2 | ASP | A | 748 | −1.704 | −19.962 | 33.246 | 1.00 | 42.46 |
| ATOM | 1726 | C | ASP | A | 748 | −2.819 | −16.171 | 32.314 | 1.00 | 20.94 |
| ATOM | 1727 | O | ASP | A | 748 | −2.240 | −15.677 | 31.351 | 1.00 | 19.01 |
| ATOM | 1728 | N | PRO | A | 749 | −2.742 | −15.637 | 33.541 | 1.00 | 18.53 |
| ATOM | 1729 | CD | PRO | A | 749 | −3.443 | −16.108 | 34.750 | 1.00 | 26.08 |
| ATOM | 1730 | CA | PRO | A | 749 | −1.958 | −14.432 | 33.825 | 1.00 | 19.83 |
| ATOM | 1731 | CB | PRO | A | 749 | −2.009 | −14.346 | 35.347 | 1.00 | 25.90 |
| ATOM | 1732 | CG | PRO | A | 749 | −3.377 | −14.896 | 35.654 | 1.00 | 25.60 |
| ATOM | 1733 | C | PRO | A | 749 | −0.528 | −14.476 | 33.286 | 1.00 | 17.96 |
| ATOM | 1734 | O | PRO | A | 749 | −0.047 | −13.506 | 32.708 | 1.00 | 18.09 |
| ATOM | 1735 | N | HIS | A | 750 | 0.149 | −15.603 | 33.475 | 1.00 | 17.27 |
| ATOM | 1736 | CA | HIS | A | 750 | 1.520 | −15.737 | 32.998 | 1.00 | 16.70 |
| ATOM | 1737 | CB | HIS | A | 750 | 2.138 | −17.039 | 33.509 | 1.00 | 14.57 |
| ATOM | 1738 | CG | HIS | A | 750 | 3.506 | −17.301 | 32.975 | 1.00 | 13.17 |
| ATOM | 1739 | CD2 | HIS | A | 750 | 4.710 | −16.780 | 33.299 | 1.00 | 15.22 |
| ATOM | 1740 | ND1 | HIS | A | 750 | 3.748 | −18.181 | 31.933 | 1.00 | 20.55 |
| ATOM | 1741 | CE1 | HIS | A | 750 | 5.030 | −18.183 | 31.649 | 1.00 | 21.44 |
| ATOM | 1742 | NE2 | HIS | A | 750 | 5.644 | −17.338 | 32.465 | 1.00 | 27.33 |
| ATOM | 1743 | C | HIS | A | 750 | 1.615 | −15.691 | 31.475 | 1.00 | 16.04 |
| ATOM | 1744 | O | HIS | A | 750 | 2.521 | −15.069 | 30.925 | 1.00 | 16.50 |
| ATOM | 1745 | N | GLU | A | 751 | 0.680 | −16.346 | 30.794 | 1.00 | 14.76 |
| ATOM | 1746 | CA | GLU | A | 751 | 0.689 | −16.359 | 29.333 | 1.00 | 14.97 |
| ATOM | 1747 | CB | GLU | A | 751 | −0.269 | −17.427 | 28.798 | 1.00 | 11.89 |
| ATOM | 1748 | CG | GLU | A | 751 | 0.173 | −18.848 | 29.123 | 1.00 | 16.94 |
| ATOM | 1749 | CD | GLU | A | 751 | −0.601 | −19.892 | 28.354 | 1.00 | 17.06 |
| ATOM | 1750 | OE1 | GLU | A | 751 | −0.804 | −19.697 | 27.140 | 1.00 | 17.15 |
| ATOM | 1751 | OE2 | GLU | A | 751 | −0.993 | −20.912 | 28.959 | 1.00 | 27.83 |
| ATOM | 1752 | C | GLU | A | 751 | 0.325 | −14.988 | 28.764 | 1.00 | 14.11 |
| ATOM | 1753 | O | GLU | A | 751 | 0.782 | −14.616 | 27.685 | 1.00 | 14.18 |
| ATOM | 1754 | N | LYS | A | 752 | −0.501 | −14.248 | 29.498 | 1.00 | 13.06 |
| ATOM | 1755 | CA | LYS | A | 752 | −0.900 | −12.910 | 29.092 | 1.00 | 11.63 |
| ATOM | 1756 | CB | LYS | A | 752 | −1.984 | −12.370 | 30.041 | 1.00 | 11.74 |
| ATOM | 1757 | CG | LYS | A | 752 | −2.200 | −10.865 | 29.981 | 1.00 | 11.33 |
| ATOM | 1758 | CD | LYS | A | 752 | −2.667 | −10.402 | 28.608 | 1.00 | 18.51 |
| ATOM | 1759 | CE | LYS | A | 752 | −3.921 | −11.138 | 28.184 | 1.00 | 31.49 |
| ATOM | 1760 | NZ | LYS | A | 752 | −4.536 | −10.523 | 26.978 | 1.00 | 47.29 |
| ATOM | 1761 | C | LYS | A | 752 | 0.362 | −12.047 | 29.176 | 1.00 | 14.58 |
| ATOM | 1762 | O | LYS | A | 752 | 0.665 | −11.293 | 28.249 | 1.00 | 11.11 |
| ATOM | 1763 | N | GLU | A | 753 | 1.093 | −12.196 | 30.270 | 1.00 | 12.46 |
| ATOM | 1764 | CA | GLU | A | 753 | 2.315 | −11.409 | 30.445 | 1.00 | 13.89 |
| ATOM | 1765 | CB | GLU | A | 753 | 2.939 | −11.745 | 31.801 | 1.00 | 15.41 |
| ATOM | 1766 | CG | GLU | A | 753 | 4.187 | −10.950 | 32.098 | 1.00 | 17.51 |
| ATOM | 1767 | CD | GLU | A | 753 | 4.946 | −11.478 | 33.300 | 1.00 | 36.21 |
| ATOM | 1768 | OE1 | GLU | A | 753 | 4.335 | −11.579 | 34.384 | 1.00 | 28.23 |
| ATOM | 1769 | OE2 | GLU | A | 753 | 6.150 | −11.782 | 33.162 | 1.00 | 36.81 |
| ATOM | 1770 | C | GLU | A | 753 | 3.267 | −11.729 | 29.313 | 1.00 | 14.45 |
| ATOM | 1771 | O | GLU | A | 753 | 3.852 | −10.839 | 28.671 | 1.00 | 11.34 |
| ATOM | 1772 | N | LEU | A | 754 | 3.441 | −13.018 | 29.035 | 1.00 | 12.90 |
| ATOM | 1773 | CA | LEU | A | 754 | 4.329 | −13.471 | 27.974 | 1.00 | 13.51 |
| ATOM | 1774 | CB | LEU | A | 754 | 4.377 | −15.011 | 27.908 | 1.00 | 13.76 |
| ATOM | 1775 | CG | LEU | A | 754 | 5.196 | −15.682 | 26.793 | 1.00 | 15.32 |
| ATOM | 1776 | CD1 | LEU | A | 754 | 6.663 | −15.286 | 26.877 | 1.00 | 15.56 |
| ATOM | 1777 | CD2 | LEU | A | 754 | 5.044 | −17.193 | 26.930 | 1.00 | 14.90 |
| ATOM | 1778 | C | LEU | A | 754 | 3.915 | −12.900 | 26.616 | 1.00 | 12.21 |
| ATOM | 1779 | O | LEU | A | 754 | 4.765 | −12.478 | 25.821 | 1.00 | 11.37 |
| ATOM | 1780 | N | PHE | A | 755 | 2.613 | −12.867 | 26.351 | 1.00 | 11.00 |
| ATOM | 1781 | CA | PHE | A | 755 | 2.119 | −12.336 | 25.088 | 1.00 | 9.92 |
| ATOM | 1782 | CB | PHE | A | 755 | 0.609 | −12.551 | 24.966 | 1.00 | 12.44 |
| ATOM | 1783 | CG | PHE | A | 755 | 0.002 | −11.867 | 23.780 | 1.00 | 11.67 |
| ATOM | 1784 | CD1 | PHE | A | 755 | 0.338 | −12.259 | 22.492 | 1.00 | 13.36 |
| ATOM | 1785 | CD2 | PHE | A | 755 | −0.858 | −10.792 | 23.948 | 1.00 | 9.93 |
| ATOM | 1786 | CE1 | PHE | A | 755 | −0.170 | −11.590 | 21.393 | 1.00 | 14.56 |
| ATOM | 1787 | CE2 | PHE | A | 755 | −1.369 | −10.118 | 22.854 | 1.00 | 13.93 |
| ATOM | 1788 | CZ | PHE | A | 755 | −1.021 | −10.517 | 21.574 | 1.00 | 18.65 |
| ATOM | 1789 | C | PHE | A | 755 | 2.431 | −10.840 | 24.972 | 1.00 | 9.95 |
| ATOM | 1790 | O | PHE | A | 755 | 2.856 | −10.367 | 23.916 | 1.00 | 10.28 |
| ATOM | 1791 | N | LEU | A | 756 | 2.225 | −10.099 | 26.057 | 1.00 | 9.98 |
| ATOM | 1792 | CA | LEU | A | 756 | 2.489 | −8.664 | 26.034 | 1.00 | 9.03 |
| ATOM | 1793 | CB | LEU | A | 756 | 2.102 | −8.017 | 27.370 | 1.00 | 9.14 |
| ATOM | 1794 | CG | LEU | A | 756 | 0.610 | −8.119 | 27.706 | 1.00 | 13.27 |
| ATOM | 1795 | CD1 | LEU | A | 756 | 0.331 | −7.487 | 29.065 | 1.00 | 21.21 |
| ATOM | 1796 | CD2 | LEU | A | 756 | −0.198 | −7.432 | 26.613 | 1.00 | 18.57 |
| ATOM | 1797 | C | LEU | A | 756 | 3.959 | −8.410 | 25.721 | 1.00 | 9.36 |
| ATOM | 1798 | O | LEU | A | 756 | 4.291 | −7.437 | 25.045 | 1.00 | 8.42 |
| ATOM | 1799 | N | ALA | A | 757 | 4.844 | −9.285 | 26.197 | 1.00 | 7.41 |
| ATOM | 1800 | CA | ALA | A | 757 | 6.267 | −9.118 | 25.915 | 1.00 | 10.17 |
| ATOM | 1801 | CB | ALA | A | 757 | 7.096 | −10.112 | 26.720 | 1.00 | 10.57 |
| ATOM | 1802 | C | ALA | A | 757 | 6.514 | −9.315 | 24.421 | 1.00 | 9.99 |
| ATOM | 1803 | O | ALA | A | 757 | 7.254 | −8.548 | 23.798 | 1.00 | 10.22 |
| ATOM | 1804 | N | MET | A | 758 | 5.890 | −10.341 | 23.845 | 1.00 | 8.41 |
| ATOM | 1805 | CA | MET | A | 758 | 6.046 | −10.627 | 22.422 | 1.00 | 8.55 |
| ATOM | 1806 | CB | MET | A | 758 | 5.348 | −11.946 | 22.061 | 1.00 | 8.57 |
| ATOM | 1807 | CG | MET | A | 758 | 5.898 | −13.175 | 22.783 | 1.00 | 9.97 |
| ATOM | 1808 | SD | MET | A | 758 | 7.613 | −13.597 | 22.367 | 1.00 | 15.99 |
| ATOM | 1809 | CE | MET | A | 758 | 7.436 | −14.226 | 20.702 | 1.00 | 36.66 |
| ATOM | 1810 | C | MET | A | 758 | 5.469 | −9.499 | 21.572 | 1.00 | 9.31 |
| ATOM | 1811 | O | MET | A | 758 | 6.035 | −9.134 | 20.534 | 1.00 | 8.67 |
| ATOM | 1812 | N | LEU | A | 759 | 4.334 | −8.962 | 22.008 | 1.00 | 10.68 |
| ATOM | 1813 | CA | LEU | A | 759 | 3.682 | −7.877 | 21.290 | 1.00 | 7.26 |
| ATOM | 1814 | CB | LEU | A | 759 | 2.348 | −7.536 | 21.954 | 1.00 | 6.70 |
| ATOM | 1815 | CG | LEU | A | 759 | 1.521 | −6.419 | 21.316 | 1.00 | 9.88 |
| ATOM | 1816 | CD1 | LEU | A | 759 | 1.217 | −6.751 | 19.858 | 1.00 | 13.46 |
| ATOM | 1817 | CD2 | LEU | A | 759 | 0.232 | −6.241 | 22.103 | 1.00 | 8.91 |
| ATOM | 1818 | C | LEU | A | 759 | 4.589 | −6.650 | 21.269 | 1.00 | 10.65 |
| ATOM | 1819 | O | LEU | A | 759 | 4.677 | −5.945 | 20.259 | 1.00 | 8.66 |
| ATOM | 1820 | N | MET | A | 760 | 5.258 | −6.388 | 22.387 | 1.00 | 8.87 |
| ATOM | 1821 | CA | MET | A | 760 | 6.168 | −5.251 | 22.454 | 1.00 | 9.10 |
| ATOM | 1822 | CB | MET | A | 760 | 6.760 | −5.114 | 23.859 | 1.00 | 9.37 |
| ATOM | 1823 | CG | MET | A | 760 | 5.831 | −4.491 | 24.889 | 1.00 | 7.94 |
| ATOM | 1824 | SD | MET | A | 760 | 5.392 | −2.762 | 24.513 | 1.00 | 11.15 |
| ATOM | 1825 | CE | MET | A | 760 | 6.981 | −1.951 | 24.748 | 1.00 | 10.58 |
| ATOM | 1826 | C | MET | A | 760 | 7.292 | −5.445 | 21.444 | 1.00 | 9.36 |
| ATOM | 1827 | O | MET | A | 760 | 7.693 | −4.501 | 20.757 | 1.00 | 8.44 |
| ATOM | 1828 | N | THR | A | 761 | 7.812 | −6.667 | 21.359 | 1.00 | 8.62 |
| ATOM | 1829 | CA | THR | A | 761 | 8.890 | −6.948 | 20.416 | 1.00 | 9.46 |
| ATOM | 1830 | CB | THR | A | 761 | 9.442 | −8.379 | 20.597 | 1.00 | 12.42 |
| ATOM | 1831 | OG1 | THR | A | 761 | 9.916 | −8.540 | 21.940 | 1.00 | 11.86 |
| ATOM | 1832 | CG2 | THR | A | 761 | 10.598 | −8.636 | 19.620 | 1.00 | 9.90 |
| ATOM | 1833 | C | THR | A | 761 | 8.389 | −6.785 | 18.984 | 1.00 | 10.31 |
| ATOM | 1834 | O | THR | A | 761 | 9.088 | −6.238 | 18.128 | 1.00 | 10.55 |
| ATOM | 1835 | N | ALA | A | 762 | 7.172 | −7.254 | 18.722 | 1.00 | 10.42 |
| ATOM | 1836 | CA | ALA | A | 762 | 6.598 | −7.148 | 17.389 | 1.00 | 7.73 |
| ATOM | 1837 | CB | ALA | A | 762 | 5.202 | −7.777 | 17.362 | 1.00 | 10.79 |
| ATOM | 1838 | C | ALA | A | 762 | 6.524 | −5.689 | 16.948 | 1.00 | 9.57 |
| ATOM | 1839 | O | ALA | A | 762 | 6.794 | −5.370 | 15.787 | 1.00 | 9.59 |
| ATOM | 1840 | N | CYS | A | 763 | 6.161 | −4.801 | 17.872 | 1.00 | 7.53 |
| ATOM | 1841 | CA | CYS | A | 763 | 6.063 | −3.378 | 17.551 | 1.00 | 7.89 |
| ATOM | 1842 | CB | CYS | A | 763 | 5.237 | −2.641 | 18.611 | 1.00 | 11.70 |
| ATOM | 1843 | SG | CYS | A | 763 | 3.503 | −3.143 | 18.670 | 1.00 | 11.19 |
| ATOM | 1844 | C | CYS | A | 763 | 7.441 | −2.744 | 17.443 | 1.00 | 9.03 |
| ATOM | 1845 | O | CYS | A | 763 | 7.689 | −1.911 | 16.574 | 1.00 | 10.26 |
| ATOM | 1846 | N | ASP | A | 764 | 8.340 | −3.157 | 18.329 | 1.00 | 8.73 |
| ATOM | 1847 | CA | ASP | A | 764 | 9.703 | −2.641 | 18.352 | 1.00 | 10.03 |
| ATOM | 1848 | CB | ASP | A | 764 | 10.448 | −3.269 | 19.541 | 1.00 | 11.85 |
| ATOM | 1849 | CG | ASP | A | 764 | 11.766 | −2.585 | 19.840 | 1.00 | 12.03 |
| ATOM | 1850 | OD1 | ASP | A | 764 | 11.963 | −1.451 | 19.361 | 1.00 | 11.46 |
| ATOM | 1851 | OD2 | ASP | A | 764 | 12.592 | −3.176 | 20.571 | 1.00 | 15.03 |
| ATOM | 1852 | C | ASP | A | 764 | 10.447 | −2.924 | 17.036 | 1.00 | 10.20 |
| ATOM | 1853 | O | ASP | A | 764 | 11.225 | −2.097 | 16.562 | 1.00 | 11.71 |
| ATOM | 1854 | N | LEU | A | 765 | 10.194 | −4.086 | 16.442 | 1.00 | 11.54 |
| ATOM | 1855 | CA | LEU | A | 765 | 10.867 | −4.474 | 15.202 | 1.00 | 11.71 |
| ATOM | 1856 | CB | LEU | A | 765 | 11.208 | −5.965 | 15.252 | 1.00 | 13.42 |
| ATOM | 1857 | CG | LEU | A | 765 | 12.017 | −6.454 | 16.453 | 1.00 | 15.02 |
| ATOM | 1858 | CD1 | LEU | A | 765 | 12.233 | −7.957 | 16.348 | 1.00 | 13.91 |
| ATOM | 1859 | CD2 | LEU | A | 765 | 13.345 | −5.721 | 16.499 | 1.00 | 16.31 |
| ATOM | 1860 | C | LEU | A | 765 | 10.049 | −4.221 | 13.941 | 1.00 | 12.10 |
| ATOM | 1861 | O | LEU | A | 765 | 10.490 | −4.560 | 12.837 | 1.00 | 11.30 |
| ATOM | 1862 | N | SER | A | 766 | 8.881 | −3.606 | 14.101 | 1.00 | 12.76 |
| ATOM | 1863 | CA | SER | A | 766 | 7.965 | −3.383 | 12.983 | 1.00 | 10.86 |
| ATOM | 1864 | CB | SER | A | 766 | 6.672 | −2.733 | 13.488 | 1.00 | 8.49 |
| ATOM | 1865 | OG | SER | A | 766 | 6.902 | −1.438 | 13.991 | 1.00 | 14.83 |
| ATOM | 1866 | C | SER | A | 766 | 8.446 | −2.657 | 11.729 | 1.00 | 7.60 |
| ATOM | 1867 | O | SER | A | 766 | 7.782 | −2.735 | 10.700 | 1.00 | 9.56 |
| ATOM | 1868 | N | ALA | A | 767 | 9.575 | −1.956 | 11.787 | 1.00 | 8.02 |
| ATOM | 1869 | CA | ALA | A | 767 | 10.073 | −1.296 | 10.581 | 1.00 | 9.45 |
| ATOM | 1870 | CB | ALA | A | 767 | 11.364 | −0.522 | 10.881 | 1.00 | 11.41 |
| ATOM | 1871 | C | ALA | A | 767 | 10.340 | −2.377 | 9.521 | 1.00 | 10.04 |
| ATOM | 1872 | O | ALA | A | 767 | 10.253 | −2.124 | 8.319 | 1.00 | 10.04 |
| ATOM | 1873 | N | ILE | A | 768 | 10.655 | −3.586 | 9.976 | 1.00 | 9.49 |
| ATOM | 1874 | CA | ILE | A | 768 | 10.942 | −4.694 | 9.066 | 1.00 | 12.99 |
| ATOM | 1875 | CB | ILE | A | 768 | 11.555 | −5.894 | 9.840 | 1.00 | 9.64 |
| ATOM | 1876 | CG2 | ILE | A | 768 | 10.473 | −6.631 | 10.612 | 1.00 | 7.91 |
| ATOM | 1877 | CG1 | ILE | A | 768 | 12.270 | −6.836 | 8.868 | 1.00 | 12.83 |
| ATOM | 1878 | CD1 | ILE | A | 768 | 13.471 | −6.199 | 8.190 | 1.00 | 15.69 |
| ATOM | 1879 | C | ILE | A | 768 | 9.704 | −5.173 | 8.296 | 1.00 | 11.57 |
| ATOM | 1880 | O | ILE | A | 768 | 9.812 | −5.986 | 7.376 | 1.00 | 12.45 |
| ATOM | 1881 | N | THR | A | 769 | 8.530 | −4.665 | 8.662 | 1.00 | 12.70 |
| ATOM | 1882 | CA | THR | A | 769 | 7.286 | −5.062 | 7.999 | 1.00 | 10.88 |
| ATOM | 1883 | CB | THR | A | 769 | 6.167 | −5.326 | 9.025 | 1.00 | 13.25 |
| ATOM | 1884 | OG1 | THR | A | 769 | 5.760 | −4.083 | 9.617 | 1.00 | 12.15 |
| ATOM | 1885 | CG2 | THR | A | 769 | 6.654 | −6.271 | 10.124 | 1.00 | 16.86 |
| ATOM | 1886 | C | THR | A | 769 | 6.762 | −3.991 | 7.036 | 1.00 | 12.18 |
| ATOM | 1887 | O | THR | A | 769 | 5.795 | −4.223 | 6.308 | 1.00 | 13.18 |
| ATOM | 1888 | N | LYS | A | 770 | 7.402 | −2.824 | 7.043 | 1.00 | 12.64 |
| ATOM | 1889 | CA | LYS | A | 770 | 6.986 | −1.686 | 6.216 | 1.00 | 10.43 |
| ATOM | 1890 | CB | LYS | A | 770 | 7.684 | −0.413 | 6.714 | 1.00 | 12.22 |
| ATOM | 1891 | CG | LYS | A | 770 | 7.375 | −0.029 | 8.165 | 1.00 | 7.87 |
| ATOM | 1892 | CD | LYS | A | 770 | 5.948 | 0.457 | 8.330 | 1.00 | 13.33 |
| ATOM | 1893 | CE | LYS | A | 770 | 5.644 | 0.766 | 9.794 | 1.00 | 14.40 |
| ATOM | 1894 | NZ | LYS | A | 770 | 4.263 | 1.293 | 9.973 | 1.00 | 14.45 |
| ATOM | 1895 | C | LYS | A | 770 | 7.247 | −1.829 | 4.716 | 1.00 | 11.91 |
| ATOM | 1896 | O | LYS | A | 770 | 8.069 | −2.638 | 4.290 | 1.00 | 13.63 |
| ATOM | 1897 | N | PRO | A | 771 | 6.529 | −1.041 | 3.892 | 1.00 | 11.52 |
| ATOM | 1898 | CD | PRO | A | 771 | 5.408 | −0.150 | 4.240 | 1.00 | 14.60 |
| ATOM | 1899 | CA | PRO | A | 771 | 6.718 | −1.096 | 2.441 | 1.00 | 12.80 |
| ATOM | 1900 | CB | PRO | A | 771 | 5.914 | 0.099 | 1.950 | 1.00 | 15.88 |
| ATOM | 1901 | CG | PRO | A | 771 | 4.751 | 0.098 | 2.886 | 1.00 | 11.15 |
| ATOM | 1902 | C | PRO | A | 771 | 8.206 | −0.933 | 2.183 | 1.00 | 14.66 |
| ATOM | 1903 | O | PRO | A | 771 | 8.880 | −0.201 | 2.908 | 1.00 | 15.01 |
| ATOM | 1904 | N | TRP | A | 772 | 8.711 | −1.604 | 1.156 | 1.00 | 14.26 |
| ATOM | 1905 | CA | TRP | A | 772 | 10.133 | −1.555 | 0.827 | 1.00 | 15.34 |
| ATOM | 1906 | CB | TRP | A | 772 | 10.367 | −2.121 | −0.578 | 1.00 | 14.10 |
| ATOM | 1907 | CG | TRP | A | 772 | 11.809 | −2.101 | −1.009 | 1.00 | 17.47 |
| ATOM | 1908 | CD2 | TRP | A | 772 | 12.931 | −2.659 | −0.307 | 1.00 | 17.61 |
| ATOM | 1909 | CE2 | TRP | A | 772 | 14.079 | −2.404 | −1.086 | 1.00 | 19.63 |
| ATOM | 1910 | CE3 | TRP | A | 772 | 13.076 | −3.348 | 0.901 | 1.00 | 16.69 |
| ATOM | 1911 | CD1 | TRP | A | 772 | 12.313 | −1.547 | −2.152 | 1.00 | 16.69 |
| ATOM | 1912 | NE1 | TRP | A | 772 | 13.675 | −1.725 | −2.204 | 1.00 | 24.41 |
| ATOM | 1913 | CZ2 | TRP | A | 772 | 15.354 | −2.816 | −0.694 | 1.00 | 24.29 |
| ATOM | 1914 | CZ3 | TRP | A | 772 | 14.344 | −3.755 | 1.289 | 1.00 | 17.48 |
| ATOM | 1915 | CH2 | TRP | A | 772 | 15.466 | −3.488 | 0.493 | 1.00 | 21.25 |
| ATOM | 1916 | C | TRP | A | 772 | 10.820 | −0.190 | 0.952 | 1.00 | 17.78 |
| ATOM | 1917 | O | TRP | A | 772 | 11.834 | −0.070 | 1.639 | 1.00 | 15.81 |
| ATOM | 1918 | N | PRO | A | 773 | 10.286 | 0.853 | 0.291 | 1.00 | 19.35 |
| ATOM | 1919 | CD | PRO | A | 773 | 9.132 | 0.877 | −0.624 | 1.00 | 18.85 |
| ATOM | 1920 | CA | PRO | A | 773 | 10.909 | 2.180 | 0.377 | 1.00 | 18.24 |
| ATOM | 1921 | CB | PRO | A | 773 | 9.926 | 3.068 | −0.382 | 1.00 | 21.17 |
| ATOM | 1922 | CG | PRO | A | 773 | 9.386 | 2.138 | −1.422 | 1.00 | 23.60 |
| ATOM | 1923 | C | PRO | A | 773 | 11.129 | 2.654 | 1.814 | 1.00 | 16.33 |
| ATOM | 1924 | O | PRO | A | 773 | 12.167 | 3.235 | 2.140 | 1.00 | 16.01 |
| ATOM | 1925 | N | ILE | A | 774 | 10.143 | 2.406 | 2.668 | 1.00 | 16.23 |
| ATOM | 1926 | CA | ILE | A | 774 | 10.228 | 2.805 | 4.066 | 1.00 | 15.30 |
| ATOM | 1927 | CB | ILE | A | 774 | 8.851 | 2.670 | 4.751 | 1.00 | 15.99 |
| ATOM | 1928 | CG2 | ILE | A | 774 | 8.977 | 2.922 | 6.250 | 1.00 | 13.24 |
| ATOM | 1929 | CG1 | ILE | A | 774 | 7.867 | 3.658 | 4.110 | 1.00 | 20.68 |
| ATOM | 1930 | CD1 | ILE | A | 774 | 6.441 | 3.501 | 4.588 | 1.00 | 21.74 |
| ATOM | 1931 | C | ILE | A | 774 | 11.264 | 1.958 | 4.804 | 1.00 | 16.31 |
| ATOM | 1932 | O | ILE | A | 774 | 12.133 | 2.491 | 5.497 | 1.00 | 15.63 |
| ATOM | 1933 | N | GLN | A | 775 | 11.173 | 0.640 | 4.650 | 1.00 | 13.80 |
| ATOM | 1934 | CA | GLN | A | 775 | 12.120 | −0.274 | 5.279 | 1.00 | 12.85 |
| ATOM | 1935 | CB | GLN | A | 775 | 11.786 | −1.727 | 4.901 | 1.00 | 13.34 |
| ATOM | 1936 | CG | GLN | A | 775 | 12.987 | −2.688 | 4.921 | 1.00 | 14.20 |
| ATOM | 1937 | CD | GLN | A | 775 | 13.582 | −2.897 | 6.305 | 1.00 | 21.70 |
| ATOM | 1938 | OE1 | GLN | A | 775 | 14.663 | −3.472 | 6.441 | 1.00 | 13.81 |
| ATOM | 1939 | NE2 | GLN | A | 775 | 12.880 | −2.440 | 7.336 | 1.00 | 14.50 |
| ATOM | 1940 | C | GLN | A | 775 | 13.549 | 0.057 | 4.877 | 1.00 | 13.13 |
| ATOM | 1941 | O | GLN | A | 775 | 14.458 | 0.060 | 5.694 | 1.00 | 12.67 |
| ATOM | 1942 | N | GLN | A | 776 | 13.749 | 0.352 | 3.590 | 1.00 | 10.04 |
| ATOM | 1943 | CA | GLN | A | 776 | 15.068 | 0.674 | 3.090 | 1.00 | 12.84 |
| ATOM | 1944 | CB | GLN | A | 776 | 15.004 | 0.820 | 1.564 | 1.00 | 19.00 |
| ATOM | 1945 | CG | GLN | A | 776 | 16.326 | 0.943 | 0.890 | 1.00 | 25.33 |
| ATOM | 1946 | CD | GLN | A | 776 | 16.172 | 0.760 | −0.595 | 1.00 | 36.37 |
| ATOM | 1947 | OE1 | GLN | A | 776 | 15.258 | 1.315 | −1.209 | 1.00 | 39.80 |
| ATOM | 1948 | NE2 | GLN | A | 776 | 17.058 | −0.023 | −1.186 | 1.00 | 41.16 |
| ATOM | 1949 | C | GLN | A | 776 | 15.627 | 1.934 | 3.738 | 1.00 | 10.61 |
| ATOM | 1950 | O | GLN | A | 776 | 16.796 | 1.988 | 4.109 | 1.00 | 13.39 |
| ATOM | 1951 | N | ARG | A | 777 | 14.776 | 2.946 | 3.922 | 1.00 | 10.52 |
| ATOM | 1952 | CA | ARG | A | 777 | 15.199 | 4.197 | 4.534 | 1.00 | 11.43 |
| ATOM | 1953 | CB | ARG | A | 777 | 14.090 | 5.253 | 4.419 | 1.00 | 15.26 |
| ATOM | 1954 | CG | ARG | A | 777 | 14.487 | 6.607 | 4.948 | 1.00 | 20.06 |
| ATOM | 1955 | CD | ARG | A | 777 | 13.343 | 7.586 | 4.765 | 1.00 | 25.19 |
| ATOM | 1956 | NE | ARG | A | 777 | 12.206 | 7.273 | 5.627 | 1.00 | 34.37 |
| ATOM | 1957 | CZ | ARG | A | 777 | 12.232 | 7.359 | 6.954 | 1.00 | 35.62 |
| ATOM | 1958 | NH1 | ARG | A | 777 | 13.340 | 7.747 | 7.575 | 1.00 | 37.87 |
| ATOM | 1959 | NH2 | ARG | A | 777 | 11.149 | 7.068 | 7.661 | 1.00 | 29.66 |
| ATOM | 1960 | C | ARG | A | 777 | 15.560 | 3.979 | 5.997 | 1.00 | 10.83 |
| ATOM | 1961 | O | ARG | A | 777 | 16.601 | 4.447 | 6.472 | 1.00 | 13.15 |
| ATOM | 1962 | N | ILE | A | 778 | 14.715 | 3.238 | 6.705 | 1.00 | 12.38 |
| ATOM | 1963 | CA | ILE | A | 778 | 14.953 | 2.944 | 8.112 | 1.00 | 13.18 |
| ATOM | 1964 | CB | ILE | A | 778 | 13.753 | 2.209 | 8.744 | 1.00 | 15.50 |
| ATOM | 1965 | CG2 | ILE | A | 778 | 14.091 | 1.788 | 10.170 | 1.00 | 13.56 |
| ATOM | 1966 | CG1 | ILE | A | 778 | 12.516 | 3.108 | 8.721 | 1.00 | 22.29 |
| ATOM | 1967 | CD1 | ILE | A | 778 | 12.685 | 4.399 | 9.478 | 1.00 | 26.81 |
| ATOM | 1968 | C | ILE | A | 778 | 16.198 | 2.086 | 8.305 | 1.00 | 12.49 |
| ATOM | 1969 | O | ILE | A | 778 | 16.963 | 2.298 | 9.245 | 1.00 | 12.70 |
| ATOM | 1970 | N | ALA | A | 779 | 16.399 | 1.117 | 7.417 | 1.00 | 12.47 |
| ATOM | 1971 | CA | ALA | A | 779 | 17.563 | 0.242 | 7.510 | 1.00 | 13.19 |
| ATOM | 1972 | CB | ALA | A | 779 | 17.538 | −0.789 | 6.391 | 1.00 | 14.05 |
| ATOM | 1973 | C | ALA | A | 779 | 18.856 | 1.051 | 7.452 | 1.00 | 13.71 |
| ATOM | 1974 | O | ALA | A | 779 | 19.839 | 0.700 | 8.106 | 1.00 | 11.99 |
| ATOM | 1975 | N | GLU | A | 780 | 18.858 | 2.131 | 6.672 | 1.00 | 14.86 |
| ATOM | 1976 | CA | GLU | A | 780 | 20.048 | 2.976 | 6.560 | 1.00 | 13.62 |
| ATOM | 1977 | CB | GLU | A | 780 | 19.860 | 4.053 | 5.487 | 1.00 | 17.29 |
| ATOM | 1978 | CG | GLU | A | 780 | 19.742 | 3.519 | 4.075 | 1.00 | 30.45 |
| ATOM | 1979 | CD | GLU | A | 780 | 19.744 | 4.628 | 3.038 | 1.00 | 42.90 |
| ATOM | 1980 | OE1 | GLU | A | 780 | 20.761 | 5.347 | 2.939 | 1.00 | 42.08 |
| ATOM | 1981 | OE2 | GLU | A | 780 | 18.728 | 4.783 | 2.326 | 1.00 | 56.88 |
| ATOM | 1982 | C | GLU | A | 780 | 20.350 | 3.649 | 7.897 | 1.00 | 13.45 |
| ATOM | 1983 | O | GLU | A | 780 | 21.506 | 3.791 | 8.277 | 1.00 | 14.75 |
| ATOM | 1984 | N | LEU | A | 781 | 19.303 | 4.074 | 8.597 | 1.00 | 12.82 |
| ATOM | 1985 | CA | LEU | A | 781 | 19.464 | 4.716 | 9.898 | 1.00 | 10.66 |
| ATOM | 1986 | CB | LEU | A | 781 | 18.113 | 5.215 | 10.410 | 1.00 | 12.60 |
| ATOM | 1987 | CG | LEU | A | 781 | 17.428 | 6.296 | 9.574 | 1.00 | 15.49 |
| ATOM | 1988 | CD1 | LEU | A | 781 | 16.101 | 6.670 | 10.215 | 1.00 | 14.52 |
| ATOM | 1989 | CD2 | LEU | A | 781 | 18.339 | 7.513 | 9.475 | 1.00 | 17.00 |
| ATOM | 1990 | C | LEU | A | 781 | 20.032 | 3.704 | 10.887 | 1.00 | 12.39 |
| ATOM | 1991 | O | LEU | A | 781 | 20.935 | 4.014 | 11.673 | 1.00 | 11.98 |
| ATOM | 1992 | N | VAL | A | 782 | 19.490 | 2.490 | 10.839 | 1.00 | 9.90 |
| ATOM | 1993 | CA | VAL | A | 782 | 19.933 | 1.418 | 11.719 | 1.00 | 9.65 |
| ATOM | 1994 | CB | VAL | A | 782 | 19.116 | 0.126 | 11.463 | 1.00 | 10.42 |
| ATOM | 1995 | CG1 | VAL | A | 782 | 19.714 | −1.029 | 12.240 | 1.00 | 13.17 |
| ATOM | 1996 | CG2 | VAL | A | 782 | 17.657 | 0.339 | 11.868 | 1.00 | 16.63 |
| ATOM | 1997 | C | VAL | A | 782 | 21.418 | 1.154 | 11.477 | 1.00 | 10.70 |
| ATOM | 1998 | O | VAL | A | 782 | 22.210 | 1.068 | 12.418 | 1.00 | 13.37 |
| ATOM | 1999 | N | ALA | A | 783 | 21.793 | 1.053 | 10.206 | 1.00 | 11.25 |
| ATOM | 2000 | CA | ALA | A | 783 | 23.185 | 0.810 | 9.839 | 1.00 | 11.83 |
| ATOM | 2001 | CB | ALA | A | 783 | 23.299 | 0.632 | 8.337 | 1.00 | 15.10 |
| ATOM | 2002 | C | ALA | A | 783 | 24.074 | 1.961 | 10.298 | 1.00 | 12.03 |
| ATOM | 2003 | O | ALA | A | 783 | 25.170 | 1.746 | 10.820 | 1.00 | 12.81 |
| ATOM | 2004 | N | THR | A | 784 | 23.599 | 3.187 | 10.099 | 1.00 | 11.94 |
| ATOM | 2005 | CA | THR | A | 784 | 24.358 | 4.359 | 10.504 | 1.00 | 10.90 |
| ATOM | 2006 | CB | THR | A | 784 | 23.572 | 5.659 | 10.211 | 1.00 | 12.78 |
| ATOM | 2007 | OG1 | THR | A | 784 | 23.359 | 5.778 | 8.797 | 1.00 | 15.62 |
| ATOM | 2008 | CG2 | THR | A | 784 | 24.341 | 6.883 | 10.706 | 1.00 | 13.70 |
| ATOM | 2009 | C | THR | A | 784 | 24.676 | 4.282 | 11.995 | 1.00 | 11.74 |
| ATOM | 2010 | O | THR | A | 784 | 25.819 | 4.490 | 12.408 | 1.00 | 10.51 |
| ATOM | 2011 | N | GLU | A | 785 | 23.670 | 3.969 | 12.805 | 1.00 | 10.01 |
| ATOM | 2012 | CA | GLU | A | 785 | 23.881 | 3.880 | 14.243 | 1.00 | 10.75 |
| ATOM | 2013 | CB | GLU | A | 785 | 22.549 | 3.669 | 14.968 | 1.00 | 7.51 |
| ATOM | 2014 | CG | GLU | A | 785 | 22.675 | 3.740 | 16.483 | 1.00 | 9.65 |
| ATOM | 2015 | CD | GLU | A | 785 | 21.335 | 3.755 | 17.178 | 1.00 | 7.52 |
| ATOM | 2016 | OE1 | GLU | A | 785 | 20.415 | 4.436 | 16.674 | 1.00 | 10.93 |
| ATOM | 2017 | OE2 | GLU | A | 785 | 21.208 | 3.099 | 18.236 | 1.00 | 12.56 |
| ATOM | 2018 | C | GLU | A | 785 | 24.847 | 2.760 | 14.612 | 1.00 | 11.99 |
| ATOM | 2019 | O | GLU | A | 785 | 25.737 | 2.948 | 15.440 | 1.00 | 11.68 |
| ATOM | 2020 | N | PHE | A | 786 | 24.671 | 1.594 | 13.997 | 1.00 | 9.83 |
| ATOM | 2021 | CA | PHE | A | 786 | 25.541 | 0.464 | 14.285 | 1.00 | 10.39 |
| ATOM | 2022 | CB | PHE | A | 786 | 25.069 | −0.794 | 13.550 | 1.00 | 11.58 |
| ATOM | 2023 | CG | PHE | A | 786 | 23.876 | −1.455 | 14.176 | 1.00 | 16.59 |
| ATOM | 2024 | CD1 | PHE | A | 786 | 23.632 | −1.327 | 15.533 | 1.00 | 18.18 |
| ATOM | 2025 | CD2 | PHE | A | 786 | 23.034 | −2.254 | 13.419 | 1.00 | 19.90 |
| ATOM | 2026 | CE1 | PHE | A | 786 | 22.570 | −1.986 | 16.125 | 1.00 | 25.35 |
| ATOM | 2027 | CE2 | PHE | A | 786 | 21.972 | −2.916 | 14.004 | 1.00 | 17.12 |
| ATOM | 2028 | CZ | PHE | A | 786 | 21.740 | −2.781 | 15.360 | 1.00 | 16.84 |
| ATOM | 2029 | C | PHE | A | 786 | 26.985 | 0.760 | 13.898 | 1.00 | 9.62 |
| ATOM | 2030 | O | PHE | A | 786 | 27.907 | 0.444 | 14.652 | 1.00 | 10.46 |
| ATOM | 2031 | N | PHE | A | 787 | 27.183 | 1.348 | 12.720 | 1.00 | 8.90 |
| ATOM | 2032 | CA | PHE | A | 787 | 28.531 | 1.680 | 12.273 | 1.00 | 9.83 |
| ATOM | 2033 | CB | PHE | A | 787 | 28.515 | 2.141 | 10.809 | 1.00 | 10.96 |
| ATOM | 2034 | CG | PHE | A | 787 | 28.116 | 1.056 | 9.849 | 1.00 | 8.28 |
| ATOM | 2035 | CD1 | PHE | A | 787 | 28.335 | −0.276 | 10.172 | 1.00 | 10.72 |
| ATOM | 2036 | CD2 | PHE | A | 787 | 27.546 | 1.357 | 8.626 | 1.00 | 11.47 |
| ATOM | 2037 | CE1 | PHE | A | 787 | 27.993 | −1.287 | 9.296 | 1.00 | 12.45 |
| ATOM | 2038 | CE2 | PHE | A | 787 | 27.201 | 0.344 | 7.739 | 1.00 | 12.21 |
| ATOM | 2039 | CZ | PHE | A | 787 | 27.424 | −0.977 | 8.075 | 1.00 | 8.97 |
| ATOM | 2040 | C | PHE | A | 787 | 29.165 | 2.747 | 13.149 | 1.00 | 12.00 |
| ATOM | 2041 | O | PHE | A | 787 | 30.387 | 2.787 | 13.311 | 1.00 | 11.70 |
| ATOM | 2042 | N | ASP | A | 788 | 28.340 | 3.620 | 13.713 | 1.00 | 10.51 |
| ATOM | 2043 | CA | ASP | A | 788 | 28.867 | 4.655 | 14.583 | 1.00 | 13.07 |
| ATOM | 2044 | CB | ASP | A | 788 | 27.770 | 5.654 | 14.953 | 1.00 | 9.28 |
| ATOM | 2045 | CG | ASP | A | 788 | 28.275 | 6.751 | 15.869 | 1.00 | 17.65 |
| ATOM | 2046 | OD1 | ASP | A | 788 | 27.862 | 6.784 | 17.046 | 1.00 | 22.26 |
| ATOM | 2047 | OD2 | ASP | A | 788 | 29.097 | 7.575 | 15.414 | 1.00 | 23.70 |
| ATOM | 2048 | CA | ASP | A | 788 | 29.427 | 3.975 | 15.832 | 1.00 | 11.61 |
| ATOM | 2049 | O | ASP | A | 788 | 30.491 | 4.349 | 16.324 | 1.00 | 11.23 |
| ATOM | 2050 | N | GLN | A | 789 | 28.719 | 2.965 | 16.338 | 1.00 | 10.39 |
| ATOM | 2051 | CA | GLN | A | 789 | 29.201 | 2.243 | 17.515 | 1.00 | 10.66 |
| ATOM | 2052 | CB | GLN | A | 789 | 28.185 | 1.199 | 17.979 | 1.00 | 14.04 |
| ATOM | 2053 | CG | GLN | A | 789 | 28.667 | 0.416 | 19.191 | 1.00 | 13.95 |
| ATOM | 2054 | CD | GLN | A | 789 | 27.696 | −0.652 | 19.641 | 1.00 | 13.88 |
| ATOM | 2055 | OE1 | GLN | A | 789 | 26.488 | −0.445 | 19.641 | 1.00 | 10.68 |
| ATOM | 2056 | NE2 | GLN | A | 789 | 28.226 | −1.799 | 20.051 | 1.00 | 10.88 |
| ATOM | 2057 | C | GLN | A | 789 | 30.509 | 1.541 | 17.155 | 1.00 | 12.25 |
| ATOM | 2058 | O | GLN | A | 789 | 31.452 | 1.504 | 17.952 | 1.00 | 12.92 |
| ATOM | 2059 | N | GLY | A | 790 | 30.552 | 0.979 | 15.950 | 1.00 | 12.27 |
| ATOM | 2060 | CA | GLY | A | 790 | 31.746 | 0.292 | 15.493 | 1.00 | 13.69 |
| ATOM | 2061 | C | GLY | A | 790 | 32.948 | 1.218 | 15.519 | 1.00 | 12.41 |
| ATOM | 2062 | O | GLY | A | 790 | 34.050 | 0.805 | 15.885 | 1.00 | 14.19 |
| ATOM | 2063 | N | ASP | A | 791 | 32.743 | 2.476 | 15.135 | 1.00 | 13.24 |
| ATOM | 2064 | CA | ASP | A | 791 | 33.837 | 3.448 | 15.135 | 1.00 | 15.89 |
| ATOM | 2065 | CB | ASP | A | 791 | 33.383 | 4.802 | 14.582 | 1.00 | 11.78 |
| ATOM | 2066 | CG | ASP | A | 791 | 33.022 | 4.748 | 13.108 | 1.00 | 11.72 |
| ATOM | 2067 | OD1 | ASP | A | 791 | 33.525 | 3.853 | 12.401 | 1.00 | 16.07 |
| ATOM | 2068 | OD2 | ASP | A | 791 | 32.249 | 5.619 | 12.657 | 1.00 | 16.56 |
| ATOM | 2069 | C | ASP | A | 791 | 34.350 | 3.653 | 16.554 | 1.00 | 15.06 |
| ATOM | 2070 | O | ASP | A | 791 | 35.555 | 3.751 | 16.794 | 1.00 | 15.97 |
| ATOM | 2071 | N | ARG | A | 792 | 33.423 | 3.723 | 17.496 | 1.00 | 16.00 |
| ATOM | 2072 | CA | ARG | A | 792 | 33.783 | 3.925 | 18.885 | 1.00 | 13.38 |
| ATOM | 2073 | CB | ARG | A | 792 | 32.544 | 4.308 | 19.680 | 1.00 | 15.67 |
| ATOM | 2074 | CG | ARG | A | 792 | 32.056 | 5.689 | 19.304 | 1.00 | 20.98 |
| ATOM | 2075 | CD | ARG | A | 792 | 30.791 | 6.043 | 20.014 | 1.00 | 22.55 |
| ATOM | 2076 | NE | ARG | A | 792 | 30.929 | 6.004 | 21.465 | 1.00 | 21.96 |
| ATOM | 2077 | CZ | ARG | A | 792 | 29.945 | 6.332 | 22.290 | 1.00 | 19.00 |
| ATOM | 2078 | NH1 | ARG | A | 792 | 28.782 | 6.726 | 21.791 | 1.00 | 18.95 |
| ATOM | 2079 | NH2 | ARG | A | 792 | 30.105 | 6.236 | 23.601 | 1.00 | 23.88 |
| ATOM | 2080 | C | ARG | A | 792 | 34.466 | 2.716 | 19.501 | 1.00 | 13.13 |
| ATOM | 2081 | O | ARG | A | 792 | 35.296 | 2.865 | 20.390 | 1.00 | 14.33 |
| ATOM | 2082 | N | GLU | A | 793 | 34.112 | 1.522 | 19.036 | 1.00 | 14.11 |
| ATOM | 2083 | CA | GLU | A | 793 | 34.730 | 0.305 | 19.545 | 1.00 | 12.55 |
| ATOM | 2084 | CB | GLU | A | 793 | 33.969 | −0.932 | 19.051 | 1.00 | 12.93 |
| ATOM | 2085 | CG | GLU | A | 793 | 32.531 | −0.986 | 19.543 | 1.00 | 15.09 |
| ATOM | 2086 | CD | GLU | A | 793 | 31.816 | −2.269 | 19.165 | 1.00 | 17.10 |
| ATOM | 2087 | OE1 | GLU | A | 793 | 32.105 | −2.807 | 18.078 | 1.00 | 14.75 |
| ATOM | 2088 | OE2 | GLU | A | 793 | 30.951 | −2.728 | 19.949 | 1.00 | 13.37 |
| ATOM | 2089 | C | GLU | A | 793 | 36.184 | 0.251 | 19.080 | 1.00 | 13.70 |
| ATOM | 2090 | O | GLU | A | 793 | 37.073 | −0.143 | 19.837 | 1.00 | 14.48 |
| ATOM | 2091 | N | ARG | A | 794 | 36.429 | 0.655 | 17.838 | 1.00 | 12.88 |
| ATOM | 2092 | CA | ARG | A | 794 | 37.792 | 0.647 | 17.318 | 1.00 | 16.50 |
| ATOM | 2093 | CB | ARG | A | 794 | 37.828 | 1.083 | 15.847 | 1.00 | 16.91 |
| ATOM | 2094 | CG | ARG | A | 794 | 37.330 | 0.047 | 14.838 | 1.00 | 16.40 |
| ATOM | 2095 | CD | ARG | A | 794 | 37.399 | 0.609 | 13.422 | 1.00 | 20.95 |
| ATOM | 2096 | NE | ARG | A | 794 | 36.790 | −0.268 | 12.422 | 1.00 | 21.25 |
| ATOM | 2097 | CZ | ARG | A | 794 | 37.323 | −1.407 | 11.996 | 1.00 | 16.41 |
| ATOM | 2098 | NH1 | ARG | A | 794 | 38.486 | −1.822 | 12.480 | 1.00 | 26.91 |
| ATOM | 2099 | NH2 | ARG | A | 794 | 36.691 | −2.134 | 11.082 | 1.00 | 18.30 |
| ATOM | 2100 | C | ARG | A | 794 | 38.648 | 1.603 | 18.141 | 1.00 | 17.48 |
| ATOM | 2101 | O | ARG | A | 794 | 39.781 | 1.287 | 18.503 | 1.00 | 18.61 |
| ATOM | 2102 | N | LYS | A | 795 | 38.087 | 2.769 | 18.443 | 1.00 | 19.00 |
| ATOM | 2103 | CA | LYS | A | 795 | 38.793 | 3.795 | 19.199 | 1.00 | 19.03 |
| ATOM | 2104 | CB | LYS | A | 795 | 38.146 | 5.161 | 18.947 | 1.00 | 22.77 |
| ATOM | 2105 | CG | LYS | A | 795 | 38.839 | 6.312 | 19.661 | 1.00 | 30.54 |
| ATOM | 2106 | CD | LYS | A | 795 | 38.128 | 7.633 | 19.410 | 1.00 | 38.45 |
| ATOM | 2107 | CE | LYS | A | 795 | 38.902 | 8.796 | 20.009 | 1.00 | 34.57 |
| ATOM | 2108 | NZ | LYS | A | 795 | 40.267 | 8.886 | 19.424 | 1.00 | 42.21 |
| ATOM | 2109 | C | LYS | A | 795 | 38.866 | 3.554 | 20.704 | 1.00 | 19.86 |
| ATOM | 2110 | O | LYS | A | 795 | 39.923 | 3.716 | 21.310 | 1.00 | 21.68 |
| ATOM | 2111 | N | GLU | A | 796 | 37.747 | 3.166 | 21.304 | 1.00 | 17.50 |
| ATOM | 2112 | CA | GLU | A | 796 | 37.696 | 2.953 | 22.745 | 1.00 | 18.20 |
| ATOM | 2113 | CB | GLU | A | 796 | 36.286 | 3.272 | 23.255 | 1.00 | 16.63 |
| ATOM | 2114 | CG | GLU | A | 796 | 35.872 | 4.722 | 23.048 | 1.00 | 19.55 |
| ATOM | 2115 | CD | GLU | A | 796 | 34.425 | 4.982 | 23.414 | 1.00 | 17.22 |
| ATOM | 2116 | OE1 | GLU | A | 796 | 33.986 | 4.520 | 24.489 | 1.00 | 17.11 |
| ATOM | 2117 | OE2 | GLU | A | 796 | 33.729 | 5.660 | 22.629 | 1.00 | 17.31 |
| ATOM | 2118 | C | GLU | A | 796 | 38.116 | 1.568 | 23.238 | 1.00 | 18.82 |
| ATOM | 2119 | O | GLU | A | 796 | 38.604 | 1.432 | 24.359 | 1.00 | 19.88 |
| ATOM | 2120 | N | LEU | A | 797 | 37.936 | 0.544 | 22.408 | 1.00 | 17.76 |
| ATOM | 2121 | CA | LEU | A | 797 | 38.279 | −0.817 | 22.809 | 1.00 | 17.55 |
| ATOM | 2122 | CB | LEU | A | 797 | 37.032 | −1.708 | 22.732 | 1.00 | 14.21 |
| ATOM | 2123 | CG | LEU | A | 797 | 35.816 | −1.230 | 23.535 | 1.00 | 21.04 |
| ATOM | 2124 | CD1 | LEU | A | 797 | 34.655 | −2.194 | 23.333 | 1.00 | 19.81 |
| ATOM | 2125 | CD2 | LEU | A | 797 | 36.177 | −1.134 | 25.008 | 1.00 | 22.47 |
| ATOM | 2126 | C | LEU | A | 797 | 39.400 | −1.441 | 21.980 | 1.00 | 18.06 |
| ATOM | 2127 | O | LEU | A | 797 | 39.899 | −2.518 | 22.312 | 1.00 | 18.10 |
| ATOM | 2128 | N | ASN | A | 798 | 39.795 | −0.760 | 20.910 | 1.00 | 17.87 |
| ATOM | 2129 | CA | ASN | A | 798 | 40.846 | −1.244 | 20.016 | 1.00 | 17.14 |
| ATOM | 2130 | CB | ASN | A | 798 | 42.202 | −1.278 | 20.732 | 1.00 | 18.53 |
| ATOM | 2131 | CG | ASN | A | 798 | 43.357 | −1.501 | 19.770 | 1.00 | 19.84 |
| ATOM | 2132 | OD1 | ASN | A | 798 | 43.497 | −0.774 | 18.788 | 1.00 | 22.65 |
| ATOM | 2133 | ND2 | ASN | A | 798 | 44.186 | −2.507 | 20.043 | 1.00 | 17.46 |
| ATOM | 2134 | C | ASN | A | 798 | 40.522 | −2.632 | 19.472 | 1.00 | 19.40 |
| ATOM | 2135 | O | ASN | A | 798 | 41.400 | −3.485 | 19.352 | 1.00 | 18.37 |
| ATOM | 2136 | N | ILE | A | 799 | 39.254 | −2.852 | 19.141 | 1.00 | 18.23 |
| ATOM | 2137 | CA | ILE | A | 799 | 38.816 | −4.134 | 18.603 | 1.00 | 20.72 |
| ATOM | 2138 | CB | ILE | A | 799 | 37.779 | −4.811 | 19.529 | 1.00 | 23.69 |
| ATOM | 2139 | CG2 | ILE | A | 799 | 38.348 | −4.970 | 20.930 | 1.00 | 25.12 |
| ATOM | 2140 | CG1 | ILE | A | 799 | 36.500 | −3.968 | 19.570 | 1.00 | 27.29 |
| ATOM | 2141 | CD1 | ILE | A | 799 | 35.381 | −4.581 | 20.373 | 1.00 | 23.09 |
| ATOM | 2142 | C | ILE | A | 799 | 38.158 | −3.926 | 17.243 | 1.00 | 20.66 |
| ATOM | 2143 | O | ILE | A | 799 | 37.636 | −2.846 | 16.956 | 1.00 | 20.48 |
| ATOM | 2144 | N | GLU | A | 800 | 38.199 | −4.951 | 16.399 | 1.00 | 20.84 |
| ATOM | 2145 | CA | GLU | A | 800 | 37.558 | −4.855 | 15.096 | 1.00 | 19.43 |
| ATOM | 2146 | CB | GLU | A | 800 | 38.162 | −5.856 | 14.109 | 1.00 | 25.89 |
| ATOM | 2147 | CG | GLU | A | 800 | 37.507 | −5.813 | 12.735 | 1.00 | 37.89 |
| ATOM | 2148 | CD | GLU | A | 800 | 38.245 | −6.637 | 11.699 | 1.00 | 48.08 |
| ATOM | 2149 | OE1 | GLU | A | 800 | 38.421 | −7.856 | 11.915 | 1.00 | 47.23 |
| ATOM | 2150 | OE2 | GLU | A | 800 | 38.647 | −6.063 | 10.663 | 1.00 | 52.27 |
| ATOM | 2151 | C | GLU | A | 800 | 36.095 | −5.188 | 15.345 | 1.00 | 16.86 |
| ATOM | 2152 | O | GLU | A | 800 | 35.774 | −6.249 | 15.879 | 1.00 | 20.00 |
| ATOM | 2153 | N | PRO | A | 801 | 35.186 | −4.277 | 14.979 | 1.00 | 16.72 |
| ATOM | 2154 | CD | PRO | A | 801 | 35.413 | −2.971 | 14.340 | 1.00 | 13.94 |
| ATOM | 2155 | CA | PRO | A | 801 | 33.755 | −4.518 | 15.188 | 1.00 | 15.17 |
| ATOM | 2156 | CB | PRO | A | 801 | 33.101 | −3.242 | 14.651 | 1.00 | 11.35 |
| ATOM | 2157 | CG | PRO | A | 801 | 34.189 | −2.204 | 14.760 | 1.00 | 18.39 |
| ATOM | 2158 | C | PRO | A | 801 | 33.249 | −5.750 | 14.446 | 1.00 | 14.88 |
| ATOM | 2159 | O | PRO | A | 801 | 33.836 | −6.168 | 13.437 | 1.00 | 14.57 |
| ATOM | 2160 | N | THR | A | 802 | 32.166 | −6.333 | 14.952 | 1.00 | 13.89 |
| ATOM | 2161 | CA | THR | A | 802 | 31.554 | −7.473 | 14.282 | 1.00 | 13.70 |
| ATOM | 2162 | CB | THR | A | 802 | 30.367 | −8.057 | 15.090 | 1.00 | 18.66 |
| ATOM | 2163 | OG1 | THR | A | 802 | 29.339 | −7.067 | 15.227 | 1.00 | 22.82 |
| ATOM | 2164 | CG2 | THR | A | 802 | 30.824 | −8.491 | 16.475 | 1.00 | 28.52 |
| ATOM | 2165 | C | THR | A | 802 | 31.028 | −6.886 | 12.974 | 1.00 | 13.06 |
| ATOM | 2166 | O | THR | A | 802 | 30.896 | −5.666 | 12.853 | 1.00 | 13.12 |
| ATOM | 2167 | N | ASP | A | 803 | 30.730 | −7.737 | 11.997 | 1.00 | 13.68 |
| ATOM | 2168 | CA | ASP | A | 803 | 30.239 | −7.260 | 10.706 | 1.00 | 13.10 |
| ATOM | 2169 | CB | ASP | A | 803 | 29.784 | −8.436 | 9.840 | 1.00 | 15.94 |
| ATOM | 2170 | CG | ASP | A | 803 | 30.942 | −9.281 | 9.340 | 1.00 | 19.70 |
| ATOM | 2171 | OD1 | ASP | A | 803 | 32.108 | −8.965 | 9.661 | 1.00 | 16.86 |
| ATOM | 2172 | OD2 | ASP | A | 803 | 30.681 | −10.265 | 8.619 | 1.00 | 17.57 |
| ATOM | 2173 | C | ASP | A | 803 | 29.092 | −6.262 | 10.829 | 1.00 | 14.26 |
| ATOM | 2174 | O | ASP | A | 803 | 29.071 | −5.239 | 10.141 | 1.00 | 15.59 |
| ATOM | 2175 | N | LEU | A | 804 | 28.142 | −6.561 | 11.706 | 1.00 | 12.76 |
| ATOM | 2176 | CA | LEU | A | 804 | 26.982 | −5.700 | 11.907 | 1.00 | 13.21 |
| ATOM | 2177 | CB | LEU | A | 804 | 26.116 | −6.251 | 13.042 | 1.00 | 14.65 |
| ATOM | 2178 | CG | LEU | A | 804 | 24.861 | −5.430 | 13.347 | 1.00 | 15.24 |
| ATOM | 2179 | CD1 | LEU | A | 804 | 24.020 | −5.317 | 12.092 | 1.00 | 16.29 |
| ATOM | 2180 | CD2 | LEU | A | 804 | 24.068 | −6.089 | 14.461 | 1.00 | 23.77 |
| ATOM | 2181 | C | LEU | A | 804 | 27.356 | −4.252 | 12.214 | 1.00 | 13.48 |
| ATOM | 2182 | O | LEU | A | 804 | 26.646 | −3.317 | 11.825 | 1.00 | 13.22 |
| ATOM | 2183 | N | MET | A | 805 | 28.476 | −4.067 | 12.904 | 1.00 | 12.28 |
| ATOM | 2184 | CA | MET | A | 805 | 28.909 | −2.725 | 13.276 | 1.00 | 11.75 |
| ATOM | 2185 | CB | MET | A | 805 | 29.141 | −2.671 | 14.790 | 1.00 | 13.28 |
| ATOM | 2186 | CG | MET | A | 805 | 27.859 | −2.865 | 15.589 | 1.00 | 33.42 |
| ATOM | 2187 | SD | MET | A | 805 | 28.125 | −3.166 | 17.338 | 1.00 | 47.91 |
| ATOM | 2188 | CE | MET | A | 805 | 26.447 | −3.244 | 17.925 | 1.00 | 28.62 |
| ATOM | 2189 | C | MET | A | 805 | 30.160 | −2.268 | 12.542 | 1.00 | 11.31 |
| ATOM | 2190 | O | MET | A | 805 | 30.761 | −1.259 | 12.908 | 1.00 | 12.42 |
| ATOM | 2191 | N | ASN | A | 806 | 30.530 | −2.992 | 11.490 | 1.00 | 11.94 |
| ATOM | 2192 | CA | ASN | A | 806 | 31.726 | −2.673 | 10.712 | 1.00 | 13.21 |
| ATOM | 2193 | CB | ASN | A | 806 | 32.537 | −3.954 | 10.471 | 1.00 | 13.42 |
| ATOM | 2194 | CG | ASN | A | 806 | 33.907 | −3.676 | 9.876 | 1.00 | 16.11 |
| ATOM | 2195 | OD1 | ASN | A | 806 | 34.209 | −2.549 | 9.484 | 1.00 | 17.92 |
| ATOM | 2196 | ND2 | ASN | A | 806 | 34.740 | −4.706 | 9.802 | 1.00 | 15.54 |
| ATOM | 2197 | C | ASN | A | 806 | 31.371 | −2.023 | 9.374 | 1.00 | 14.20 |
| ATOM | 2198 | O | ASN | A | 806 | 30.988 | −2.715 | 8.430 | 1.00 | 14.08 |
| ATOM | 2199 | N | ARG | A | 807 | 31.508 | −0.699 | 9.296 | 1.00 | 12.56 |
| ATOM | 2200 | CA | ARG | A | 807 | 31.189 | 0.037 | 8.075 | 1.00 | 14.91 |
| ATOM | 2201 | CB | ARG | A | 807 | 31.410 | 1.543 | 8.275 | 1.00 | 16.99 |
| ATOM | 2202 | CG | ARG | A | 807 | 30.891 | 2.385 | 7.116 | 1.00 | 25.83 |
| ATOM | 2203 | CD | ARG | A | 807 | 31.162 | 3.881 | 7.280 | 1.00 | 30.30 |
| ATOM | 2204 | NE | ARG | A | 807 | 30.520 | 4.470 | 8.457 | 1.00 | 20.02 |
| ATOM | 2205 | CZ | ARG | A | 807 | 31.078 | 4.525 | 9.662 | 1.00 | 16.27 |
| ATOM | 2206 | NH1 | ARG | A | 807 | 32.290 | 4.025 | 9.854 | 1.00 | 20.90 |
| ATOM | 2207 | NH2 | ARG | A | 807 | 30.431 | 5.095 | 10.670 | 1.00 | 15.75 |
| ATOM | 2208 | C | ARG | A | 807 | 32.021 | −0.442 | 6.889 | 1.00 | 13.89 |
| ATOM | 2209 | O | ARG | A | 807 | 31.573 | −0.392 | 5.736 | 1.00 | 13.84 |
| ATOM | 2210 | N | GLU | A | 808 | 33.228 | −0.910 | 7.177 | 1.00 | 11.68 |
| ATOM | 2211 | CA | GLU | A | 808 | 34.122 | −1.397 | 6.137 | 1.00 | 17.06 |
| ATOM | 2212 | CB | GLU | A | 808 | 35.502 | −1.675 | 6.731 | 1.00 | 16.87 |
| ATOM | 2213 | CG | GLU | A | 808 | 36.256 | −0.394 | 7.085 | 1.00 | 22.64 |
| ATOM | 2214 | CD | GLU | A | 808 | 37.556 | −0.645 | 7.819 | 1.00 | 24.85 |
| ATOM | 2215 | OE1 | GLU | A | 808 | 38.293 | −1.576 | 7.430 | 1.00 | 26.32 |
| ATOM | 2216 | OE2 | GLU | A | 808 | 37.846 | 0.100 | 8.780 | 1.00 | 36.15 |
| ATOM | 2217 | C | GLU | A | 808 | 33.565 | −2.641 | 5.461 | 1.00 | 15.58 |
| ATOM | 2218 | O | GLU | A | 808 | 33.929 | −2.956 | 4.329 | 1.00 | 15.69 |
| ATOM | 2219 | N | LYS | A | 809 | 32.677 | −3.343 | 6.156 | 1.00 | 14.84 |
| ATOM | 2220 | CA | LYS | A | 809 | 32.062 | −4.544 | 5.600 | 1.00 | 16.67 |
| ATOM | 2221 | CB | LYS | A | 809 | 32.381 | −5.765 | 6.478 | 1.00 | 12.30 |
| ATOM | 2222 | CG | LYS | A | 809 | 33.845 | −6.184 | 6.405 | 1.00 | 13.87 |
| ATOM | 2223 | CD | LYS | A | 809 | 34.151 | −7.408 | 7.259 | 1.00 | 14.82 |
| ATOM | 2224 | CE | LYS | A | 809 | 33.430 | −8.641 | 6.745 | 1.00 | 25.64 |
| ATOM | 2225 | NZ | LYS | A | 809 | 33.789 | −9.854 | 7.534 | 1.00 | 21.95 |
| ATOM | 2226 | C | LYS | A | 809 | 30.555 | −4.363 | 5.445 | 1.00 | 15.95 |
| ATOM | 2227 | O | LYS | A | 809 | 29.770 | −5.281 | 5.693 | 1.00 | 18.05 |
| ATOM | 2228 | N | LYS | A | 810 | 30.156 | −3.167 | 5.022 | 1.00 | 15.13 |
| ATOM | 2229 | CA | LYS | A | 810 | 28.742 | −2.867 | 4.828 | 1.00 | 16.24 |
| ATOM | 2230 | CB | LYS | A | 810 | 28.549 | −1.383 | 4.494 | 1.00 | 14.35 |
| ATOM | 2231 | CG | LYS | A | 810 | 29.347 | −0.869 | 3.312 | 1.00 | 24.48 |
| ATOM | 2232 | CD | LYS | A | 810 | 29.222 | 0.647 | 3.226 | 1.00 | 29.64 |
| ATOM | 2233 | CE | LYS | A | 810 | 29.994 | 1.224 | 2.049 | 1.00 | 35.68 |
| ATOM | 2234 | NZ | LYS | A | 810 | 29.432 | 0.786 | 0.746 | 1.00 | 42.39 |
| ATOM | 2235 | C | LYS | A | 810 | 28.139 | −3.744 | 3.734 | 1.00 | 14.42 |
| ATOM | 2236 | O | LYS | A | 810 | 26.921 | −3.843 | 3.607 | 1.00 | 14.44 |
| ATOM | 2237 | N | ASN | A | 811 | 28.995 | −4.389 | 2.950 | 1.00 | 13.62 |
| ATOM | 2238 | CA | ASN | A | 811 | 28.511 | −5.270 | 1.893 | 1.00 | 13.60 |
| ATOM | 2239 | CB | ASN | A | 811 | 29.680 | −5.746 | 1.016 | 1.00 | 18.14 |
| ATOM | 2240 | CG | ASN | A | 811 | 30.815 | −6.340 | 1.826 | 1.00 | 21.65 |
| ATOM | 2241 | OD1 | ASN | A | 811 | 30.936 | −7.559 | 1.958 | 1.00 | 17.54 |
| ATOM | 2242 | ND2 | ASN | A | 811 | 31.651 | −5.472 | 2.388 | 1.00 | 13.93 |
| ATOM | 2243 | C | ASN | A | 811 | 27.777 | −6.474 | 2.487 | 1.00 | 14.60 |
| ATOM | 2244 | O | ASN | A | 811 | 27.084 | −7.197 | 1.777 | 1.00 | 13.25 |
| ATOM | 2245 | N | LYS | A | 812 | 27.922 | −6.677 | 3.796 | 1.00 | 14.05 |
| ATOM | 2246 | CA | LYS | A | 812 | 27.279 | −7.797 | 4.478 | 1.00 | 15.17 |
| ATOM | 2247 | CB | LYS | A | 812 | 28.057 | −8.150 | 5.753 | 1.00 | 15.23 |
| ATOM | 2248 | CG | LYS | A | 812 | 29.401 | −8.803 | 5.500 | 1.00 | 20.27 |
| ATOM | 2249 | CD | LYS | A | 812 | 29.198 | −10.137 | 4.817 | 1.00 | 28.44 |
| ATOM | 2250 | CE | LYS | A | 812 | 30.497 | −10.886 | 4.658 | 1.00 | 35.44 |
| ATOM | 2251 | NZ | LYS | A | 812 | 30.257 | −12.223 | 4.048 | 1.00 | 38.51 |
| ATOM | 2252 | C | LYS | A | 812 | 25.818 | −7.542 | 4.859 | 1.00 | 15.78 |
| ATOM | 2253 | O | LYS | A | 812 | 25.109 | −8.475 | 5.258 | 1.00 | 13.98 |
| ATOM | 2254 | N | ILE | A | 813 | 25.366 | −6.297 | 4.724 | 1.00 | 14.64 |
| ATOM | 2255 | CA | ILE | A | 813 | 24.012 | −5.964 | 5.144 | 1.00 | 14.83 |
| ATOM | 2256 | CB | ILE | A | 813 | 23.770 | −4.460 | 5.007 | 1.00 | 15.21 |
| ATOM | 2257 | CG2 | ILE | A | 813 | 22.264 | −4.158 | 5.091 | 1.00 | 15.81 |
| ATOM | 2258 | CG1 | ILE | A | 813 | 24.616 | −3.740 | 6.073 | 1.00 | 13.99 |
| ATOM | 2259 | CD1 | ILE | A | 813 | 24.445 | −2.265 | 6.070 | 1.00 | 27.43 |
| ATOM | 2260 | C | ILE | A | 813 | 22.862 | −6.792 | 4.546 | 1.00 | 14.67 |
| ATOM | 2261 | O | ILE | A | 813 | 22.042 | −7.309 | 5.270 | 1.00 | 13.44 |
| ATOM | 2262 | N | PRO | A | 814 | 22.807 | −6.936 | 3.224 | 1.00 | 12.07 |
| ATOM | 2263 | CD | PRO | A | 814 | 23.603 | −6.274 | 2.191 | 1.00 | 14.86 |
| ATOM | 2264 | CA | PRO | A | 814 | 21.709 | −7.724 | 2.623 | 1.00 | 14.39 |
| ATOM | 2265 | CB | PRO | A | 814 | 22.060 | −7.714 | 1.152 | 1.00 | 13.45 |
| ATOM | 2266 | CG | PRO | A | 814 | 22.693 | −6.351 | 1.001 | 1.00 | 14.67 |
| ATOM | 2267 | C | PRO | A | 814 | 21.605 | −9.144 | 3.185 | 1.00 | 11.82 |
| ATOM | 2268 | O | PRO | A | 814 | 20.525 | −9.603 | 3.570 | 1.00 | 12.87 |
| ATOM | 2269 | N | SER | A | 815 | 22.730 | −9.846 | 3.215 | 1.00 | 11.71 |
| ATOM | 2270 | CA | SER | A | 815 | 22.730 | −11.205 | 3.729 | 1.00 | 13.13 |
| ATOM | 2271 | CB | SER | A | 815 | 24.102 | −11.863 | 3.526 | 1.00 | 14.66 |
| ATOM | 2272 | OG | SER | A | 815 | 25.124 | −11.185 | 4.233 | 1.00 | 27.24 |
| ATOM | 2273 | C | SER | A | 815 | 22.366 | −11.220 | 5.212 | 1.00 | 14.09 |
| ATOM | 2274 | O | SER | A | 815 | 21.677 | −12.120 | 5.682 | 1.00 | 14.61 |
| ATOM | 2275 | N | MET | A | 816 | 22.809 | −10.211 | 5.937 | 1.00 | 14.82 |
| ATOM | 2276 | CA | MET | A | 816 | 22.498 | −10.197 | 7.353 | 1.00 | 14.08 |
| ATOM | 2277 | CB | MET | A | 816 | 23.449 | −9.254 | 8.079 | 1.00 | 16.95 |
| ATOM | 2278 | CG | MET | A | 816 | 24.829 | −9.872 | 8.232 | 1.00 | 19.08 |
| ATOM | 2279 | SD | MET | A | 816 | 26.019 | −8.905 | 9.168 | 1.00 | 23.16 |
| ATOM | 2280 | CE | MET | A | 816 | 25.502 | −9.310 | 10.812 | 1.00 | 29.41 |
| ATOM | 2281 | C | MET | A | 816 | 21.057 | −9.838 | 7.628 | 1.00 | 12.78 |
| ATOM | 2282 | O | MET | A | 816 | 20.477 | −10.281 | 8.613 | 1.00 | 13.06 |
| ATOM | 2283 | N | GLN | A | 817 | 20.464 | −9.010 | 6.765 | 1.00 | 11.62 |
| ATOM | 2284 | CA | GLN | A | 817 | 19.061 | −8.637 | 6.915 | 1.00 | 11.33 |
| ATOM | 2285 | CB | GLN | A | 817 | 18.666 | −7.547 | 5.899 | 1.00 | 12.94 |
| ATOM | 2286 | CG | GLN | A | 817 | 19.186 | −6.179 | 6.283 | 1.00 | 11.95 |
| ATOM | 2287 | CD | GLN | A | 817 | 18.469 | −5.624 | 7.500 | 1.00 | 17.94 |
| ATOM | 2288 | OE1 | GLN | A | 817 | 18.544 | −6.183 | 8.595 | 1.00 | 34.56 |
| ATOM | 2289 | NE2 | GLN | A | 817 | 17.759 | −4.523 | 7.309 | 1.00 | 34.13 |
| ATOM | 2290 | C | GLN | A | 817 | 18.202 | −9.882 | 6.714 | 1.00 | 13.31 |
| ATOM | 2291 | O | GLN | A | 817 | 17.254 | −10.116 | 7.472 | 1.00 | 10.14 |
| ATOM | 2292 | N | VAL | A | 818 | 18.537 | −10.698 | 5.715 | 1.00 | 12.18 |
| ATOM | 2293 | CA | VAL | A | 818 | 17.774 | −11.918 | 5.479 | 1.00 | 11.53 |
| ATOM | 2294 | CB | VAL | A | 818 | 18.252 | −12.659 | 4.210 | 1.00 | 13.56 |
| ATOM | 2295 | CG1 | VAL | A | 818 | 17.500 | −13.972 | 4.063 | 1.00 | 10.58 |
| ATOM | 2296 | CG2 | VAL | A | 818 | 18.032 | −11.781 | 2.982 | 1.00 | 17.92 |
| ATOM | 2297 | C | VAL | A | 818 | 17.903 | −12.858 | 6.678 | 1.00 | 11.51 |
| ATOM | 2298 | O | VAL | A | 818 | 16.930 | −13.494 | 7.090 | 1.00 | 12.55 |
| ATOM | 2299 | N | GLY | A | 819 | 19.110 | −12.934 | 7.231 | 1.00 | 11.44 |
| ATOM | 2300 | CA | GLY | A | 819 | 19.356 | −13.791 | 8.376 | 1.00 | 12.74 |
| ATOM | 2301 | C | GLY | A | 819 | 18.556 | −13.335 | 9.579 | 1.00 | 13.96 |
| ATOM | 2302 | O | GLY | A | 819 | 18.068 | −14.149 | 10.360 | 1.00 | 13.49 |
| ATOM | 2303 | N | PHE | A | 820 | 18.430 | −12.022 | 9.725 | 1.00 | 14.20 |
| ATOM | 2304 | CA | PHE | A | 820 | 17.682 | −11.426 | 10.825 | 1.00 | 11.70 |
| ATOM | 2305 | CB | PHE | A | 820 | 17.927 | −9.913 | 10.833 | 1.00 | 11.32 |
| ATOM | 2306 | CG | PHE | A | 820 | 17.122 | −9.167 | 11.858 | 1.00 | 9.99 |
| ATOM | 2307 | CD1 | PHE | A | 820 | 17.268 | −9.436 | 13.209 | 1.00 | 23.52 |
| ATOM | 2308 | CD2 | PHE | A | 820 | 16.225 | −8.184 | 11.466 | 1.00 | 17.26 |
| ATOM | 2309 | CE1 | PHE | A | 820 | 16.529 | −8.740 | 14.151 | 1.00 | 19.87 |
| ATOM | 2310 | CE2 | PHE | A | 820 | 15.485 | −7.488 | 12.403 | 1.00 | 16.59 |
| ATOM | 2311 | CZ | PHE | A | 820 | 15.640 | −7.767 | 13.746 | 1.00 | 17.66 |
| ATOM | 2312 | C | PHE | A | 820 | 16.191 | −11.731 | 10.651 | 1.00 | 12.70 |
| ATOM | 2313 | O | PHE | A | 820 | 15.498 | −12.103 | 11.604 | 1.00 | 10.72 |
| ATOM | 2314 | N | ILE | A | 821 | 15.697 | −11.571 | 9.429 | 1.00 | 10.04 |
| ATOM | 2315 | CA | ILE | A | 821 | 14.298 | −11.850 | 9.150 | 1.00 | 9.29 |
| ATOM | 2316 | CB | ILE | A | 821 | 13.957 | −11.540 | 7.679 | 1.00 | 10.14 |
| ATOM | 2317 | CG2 | ILE | A | 821 | 12.617 | −12.166 | 7.307 | 1.00 | 12.87 |
| ATOM | 2318 | CG1 | ILE | A | 821 | 13.931 | −10.022 | 7.479 | 1.00 | 12.03 |
| ATOM | 2319 | CD1 | ILE | A | 821 | 13.795 | −9.580 | 6.037 | 1.00 | 14.77 |
| ATOM | 2320 | C | ILE | A | 821 | 13.991 | −13.311 | 9.462 | 1.00 | 11.67 |
| ATOM | 2321 | O | ILE | A | 821 | 13.012 | −13.617 | 10.140 | 1.00 | 11.27 |
| ATOM | 2322 | N | ASP | A | 822 | 14.841 | −14.215 | 8.985 | 1.00 | 11.97 |
| ATOM | 2323 | CA | ASP | A | 822 | 14.634 | −15.637 | 9.237 | 1.00 | 14.30 |
| ATOM | 2324 | CB | ASP | A | 822 | 15.630 | −16.479 | 8.428 | 1.00 | 17.59 |
| ATOM | 2325 | CG | ASP | A | 822 | 15.317 | −16.499 | 6.942 | 1.00 | 17.92 |
| ATOM | 2326 | OD1 | ASP | A | 822 | 14.177 | −16.153 | 6.564 | 1.00 | 15.98 |
| ATOM | 2327 | OD2 | ASP | A | 822 | 16.212 | −16.880 | 6.152 | 1.00 | 13.97 |
| ATOM | 2328 | C | ASP | A | 822 | 14.759 | −16.021 | 10.714 | 1.00 | 15.42 |
| ATOM | 2329 | O | ASP | A | 822 | 13.876 | −16.671 | 11.275 | 1.00 | 16.53 |
| ATOM | 2330 | N | ALA | A | 823 | 15.849 | −15.608 | 11.345 | 1.00 | 13.55 |
| ATOM | 2331 | CA | ALA | A | 823 | 16.101 | −15.971 | 12.737 | 1.00 | 15.03 |
| ATOM | 2332 | CB | ALA | A | 823 | 17.580 | −15.756 | 13.058 | 1.00 | 13.84 |
| ATOM | 2333 | C | ALA | A | 823 | 15.254 | −15.299 | 13.810 | 1.00 | 15.13 |
| ATOM | 2334 | O | ALA | A | 823 | 14.907 | −15.928 | 14.810 | 1.00 | 17.91 |
| ATOM | 2335 | N | ILE | A | 824 | 14.914 | −14.032 | 13.606 | 1.00 | 13.91 |
| ATOM | 2336 | CA | ILE | A | 824 | 14.169 | −13.281 | 14.611 | 1.00 | 14.87 |
| ATOM | 2337 | CB | ILE | A | 824 | 14.929 | −11.971 | 14.966 | 1.00 | 16.51 |
| ATOM | 2338 | CG2 | ILE | A | 824 | 14.085 | −11.106 | 15.891 | 1.00 | 11.92 |
| ATOM | 2339 | CG1 | ILE | A | 824 | 16.291 | −12.293 | 15.589 | 1.00 | 19.03 |
| ATOM | 2340 | CD1 | ILE | A | 824 | 16.211 | −12.966 | 16.938 | 1.00 | 17.79 |
| ATOM | 2341 | C | ILE | A | 824 | 12.736 | −12.872 | 14.271 | 1.00 | 15.38 |
| ATOM | 2342 | O | ILE | A | 824 | 11.817 | −13.051 | 15.077 | 1.00 | 15.58 |
| ATOM | 2343 | N | CYS | A | 825 | 12.548 | −12.332 | 13.074 | 1.00 | 11.66 |
| ATOM | 2344 | CA | CYS | A | 825 | 11.248 | −11.798 | 12.682 | 1.00 | 10.83 |
| ATOM | 2345 | CB | CYS | A | 825 | 11.470 | −10.717 | 11.629 | 1.00 | 14.06 |
| ATOM | 2346 | SG | CYS | A | 825 | 12.684 | −9.498 | 12.156 | 1.00 | 12.72 |
| ATOM | 2347 | C | CYS | A | 825 | 10.094 | −12.685 | 12.231 | 1.00 | 12.83 |
| ATOM | 2348 | O | CYS | A | 825 | 9.003 | −12.606 | 12.792 | 1.00 | 11.78 |
| ATOM | 2349 | N | LEU | A | 826 | 10.312 | −13.508 | 11.213 | 1.00 | 10.64 |
| ATOM | 2350 | CA | LEU | A | 826 | 9.230 | −14.343 | 10.699 | 1.00 | 12.29 |
| ATOM | 2351 | CB | LEU | A | 826 | 9.759 | −15.291 | 9.621 | 1.00 | 17.45 |
| ATOM | 2352 | CG | LEU | A | 826 | 10.132 | −14.585 | 8.314 | 1.00 | 14.44 |
| ATOM | 2353 | CD1 | LEU | A | 826 | 10.756 | −15.585 | 7.355 | 1.00 | 19.69 |
| ATOM | 2354 | CD2 | LEU | A | 826 | 8.884 | −13.965 | 7.693 | 1.00 | 17.13 |
| ATOM | 2355 | C | LEU | A | 826 | 8.428 | −15.131 | 11.735 | 1.00 | 11.75 |
| ATOM | 2356 | O | LEU | A | 826 | 7.194 | −15.094 | 11.728 | 1.00 | 12.87 |
| ATOM | 2357 | N | GLN | A | 827 | 9.115 | −15.842 | 12.620 | 1.00 | 9.56 |
| ATOM | 2358 | CA | GLN | A | 827 | 8.426 | −16.637 | 13.632 | 1.00 | 10.96 |
| ATOM | 2359 | CB | GLN | A | 827 | 9.438 | −17.368 | 14.507 | 1.00 | 9.95 |
| ATOM | 2360 | CG | GLN | A | 827 | 10.207 | −18.465 | 13.805 | 1.00 | 14.60 |
| ATOM | 2361 | CD | GLN | A | 827 | 11.365 | −18.949 | 14.646 | 1.00 | 27.21 |
| ATOM | 2362 | OE1 | GLN | A | 827 | 12.420 | −18.312 | 14.695 | 1.00 | 20.68 |
| ATOM | 2363 | NE2 | GLN | A | 827 | 11.169 | −20.068 | 15.335 | 1.00 | 24.19 |
| ATOM | 2364 | C | GLN | A | 827 | 7.516 | −15.798 | 14.524 | 1.00 | 13.14 |
| ATOM | 2365 | O | GLN | A | 827 | 6.428 | −16.235 | 14.906 | 1.00 | 13.21 |
| ATOM | 2366 | N | LEU | A | 828 | 7.966 | −14.596 | 14.862 | 1.00 | 10.02 |
| ATOM | 2367 | CA | LEU | A | 828 | 7.182 | −13.713 | 15.720 | 1.00 | 10.78 |
| ATOM | 2368 | CB | LEU | A | 828 | 8.037 | −12.521 | 16.166 | 1.00 | 9.67 |
| ATOM | 2369 | CG | LEU | A | 828 | 7.315 | −11.431 | 16.966 | 1.00 | 14.41 |
| ATOM | 2370 | CD1 | LEU | A | 828 | 6.653 | −12.048 | 18.185 | 1.00 | 16.13 |
| ATOM | 2371 | CD2 | LEU | A | 828 | 8.300 | −10.351 | 17.389 | 1.00 | 14.03 |
| ATOM | 2372 | C | LEU | A | 828 | 5.902 | −13.218 | 15.036 | 1.00 | 11.55 |
| ATOM | 2373 | O | LEU | A | 828 | 4.814 | −13.290 | 15.609 | 1.00 | 12.50 |
| ATOM | 2374 | N | TYR | A | 829 | 6.024 | −12.717 | 13.812 | 1.00 | 10.38 |
| ATOM | 2375 | CA | TYR | A | 829 | 4.851 | −12.217 | 13.109 | 1.00 | 11.38 |
| ATOM | 2376 | CB | TYR | A | 829 | 5.286 | −11.392 | 11.892 | 1.00 | 9.07 |
| ATOM | 2377 | CG | TYR | A | 829 | 5.967 | −10.112 | 12.324 | 1.00 | 13.32 |
| ATOM | 2378 | CD1 | TYR | A | 829 | 5.298 | −9.189 | 13.118 | 1.00 | 8.62 |
| ATOM | 2379 | CE1 | TYR | A | 829 | 5.926 | −8.037 | 13.569 | 1.00 | 10.58 |
| ATOM | 2380 | CD2 | TYR | A | 829 | 7.290 | −9.849 | 11.989 | 1.00 | 10.78 |
| ATOM | 2381 | CE2 | TYR | A | 829 | 7.930 | −8.699 | 12.440 | 1.00 | 8.02 |
| ATOM | 2382 | CZ | TYR | A | 829 | 7.241 | −7.800 | 13.228 | 1.00 | 8.64 |
| ATOM | 2383 | OH | TYR | A | 829 | 7.867 | −6.659 | 13.688 | 1.00 | 10.63 |
| ATOM | 2384 | C | TYR | A | 829 | 3.908 | −13.349 | 12.731 | 1.00 | 12.07 |
| ATOM | 2385 | O | TYR | A | 829 | 2.704 | −13.140 | 12.594 | 1.00 | 14.07 |
| ATOM | 2386 | N | GLU | A | 830 | 4.452 | −14.555 | 12.585 | 1.00 | 14.19 |
| ATOM | 2387 | CA | GLU | A | 830 | 3.614 | −15.709 | 12.274 | 1.00 | 14.53 |
| ATOM | 2388 | CB | GLU | A | 830 | 4.469 | −16.919 | 11.881 | 1.00 | 15.21 |
| ATOM | 2389 | CG | GLU | A | 830 | 5.196 | −16.751 | 10.557 | 1.00 | 18.48 |
| ATOM | 2390 | CD | GLU | A | 830 | 6.057 | −17.948 | 10.208 | 1.00 | 27.35 |
| ATOM | 2391 | OE1 | GLU | A | 830 | 6.422 | −18.701 | 11.133 | 1.00 | 25.55 |
| ATOM | 2392 | OE2 | GLU | A | 830 | 6.380 | −18.127 | 9.014 | 1.00 | 25.64 |
| ATOM | 2393 | C | GLU | A | 830 | 2.806 | −16.027 | 13.531 | 1.00 | 15.34 |
| ATOM | 2394 | O | GLU | A | 830 | 1.594 | −16.245 | 13.462 | 1.00 | 15.05 |
| ATOM | 2395 | N | ALA | A | 831 | 3.484 | −16.037 | 14.679 | 1.00 | 12.88 |
| ATOM | 2396 | CA | ALA | A | 831 | 2.836 | −16.319 | 15.958 | 1.00 | 13.60 |
| ATOM | 2397 | CB | ALA | A | 831 | 3.877 | −16.341 | 17.080 | 1.00 | 14.75 |
| ATOM | 2398 | C | ALA | A | 831 | 1.764 | −15.279 | 16.264 | 1.00 | 14.36 |
| ATOM | 2399 | O | ALA | A | 831 | 0.666 | −15.617 | 16.714 | 1.00 | 13.70 |
| ATOM | 2400 | N | LEU | A | 832 | 2.085 | −14.010 | 16.026 | 1.00 | 11.70 |
| ATOM | 2401 | CA | LEU | A | 832 | 1.135 | −12.935 | 16.276 | 1.00 | 12.90 |
| ATOM | 2402 | CB | LEU | A | 832 | 1.800 | −11.577 | 16.030 | 1.00 | 12.02 |
| ATOM | 2403 | CG | LEU | A | 832 | 0.935 | −10.334 | 16.256 | 1.00 | 13.07 |
| ATOM | 2404 | CD1 | LEU | A | 832 | 0.367 | −10.354 | 17.670 | 1.00 | 16.97 |
| ATOM | 2405 | CD2 | LEU | A | 832 | 1.764 | −9.076 | 16.018 | 1.00 | 12.70 |
| ATOM | 2406 | C | LEU | A | 832 | −0.093 | −13.098 | 15.382 | 1.00 | 13.07 |
| ATOM | 2407 | O | LEU | A | 832 | −1.221 | −12.851 | 15.811 | 1.00 | 12.63 |
| ATOM | 2408 | N | THR | A | 833 | 0.125 | −13.522 | 14.140 | 1.00 | 10.33 |
| ATOM | 2409 | CA | THR | A | 833 | −0.985 | −13.720 | 13.213 | 1.00 | 11.65 |
| ATOM | 2410 | CB | THR | A | 833 | −0.471 | −14.019 | 11.798 | 1.00 | 15.54 |
| ATOM | 2411 | OG1 | THR | A | 833 | 0.250 | −12.881 | 11.309 | 1.00 | 13.44 |
| ATOM | 2412 | CG2 | THR | A | 833 | −1.631 | −14.319 | 10.856 | 1.00 | 20.09 |
| ATOM | 2413 | C | THR | A | 833 | −1.856 | −14.875 | 13.709 | 1.00 | 13.24 |
| ATOM | 2414 | O | THR | A | 833 | −3.070 | −14.879 | 13.512 | 1.00 | 14.62 |
| ATOM | 2415 | N | HIS | A | 834 | −1.233 | −15.855 | 14.358 | 1.00 | 12.04 |
| ATOM | 2416 | CA | HIS | A | 834 | −1.974 | −16.988 | 14.901 | 1.00 | 15.35 |
| ATOM | 2417 | CB | HIS | A | 834 | −1.010 | −18.045 | 15.451 | 1.00 | 17.31 |
| ATOM | 2418 | CG | HIS | A | 834 | −1.686 | −19.161 | 16.174 | 1.00 | 21.70 |
| ATOM | 2419 | CD2 | HIS | A | 834 | −2.005 | −20.422 | 15.792 | 1.00 | 27.35 |
| ATOM | 2420 | ND1 | HIS | A | 834 | −2.158 | −19.040 | 17.471 | 1.00 | 27.23 |
| ATOM | 2421 | CE1 | HIS | A | 834 | −2.728 | −20.162 | 17.843 | 1.00 | 22.73 |
| ATOM | 2422 | NE2 | HIS | A | 834 | −2.650 | −21.026 | 16.836 | 1.00 | 26.03 |
| ATOM | 2423 | C | HIS | A | 834 | −2.907 | −16.495 | 16.011 | 1.00 | 16.43 |
| ATOM | 2424 | O | HIS | A | 834 | −4.061 | −16.917 | 16.101 | 1.00 | 16.32 |
| ATOM | 2425 | N | VAL | A | 835 | −2.402 | −15.600 | 16.852 | 1.00 | 14.39 |
| ATOM | 2426 | CA | VAL | A | 835 | −3.201 | −15.042 | 17.940 | 1.00 | 16.17 |
| ATOM | 2427 | CB | VAL | A | 835 | −2.329 | −14.177 | 18.879 | 1.00 | 20.06 |
| ATOM | 2428 | CG1 | VAL | A | 835 | −3.203 | −13.433 | 19.878 | 1.00 | 26.18 |
| ATOM | 2429 | CG2 | VAL | A | 835 | −1.336 | −15.067 | 19.616 | 1.00 | 22.85 |
| ATOM | 2430 | C | VAL | A | 835 | −4.345 | −14.196 | 17.376 | 1.00 | 18.30 |
| ATOM | 2431 | O | VAL | A | 835 | −5.462 | −14.219 | 17.899 | 1.00 | 16.68 |
| ATOM | 2432 | N | SER | A | 836 | −4.061 | −13.461 | 16.302 | 1.00 | 15.55 |
| ATOM | 2433 | CA | SER | A | 836 | −5.060 | −12.620 | 15.646 | 1.00 | 15.62 |
| ATOM | 2434 | CB | SER | A | 836 | −5.081 | −11.222 | 16.267 | 1.00 | 18.44 |
| ATOM | 2435 | OG | SER | A | 836 | −6.092 | −10.430 | 15.665 | 1.00 | 24.05 |
| ATOM | 2436 | C | SER | A | 836 | −4.744 | −12.516 | 14.153 | 1.00 | 15.03 |
| ATOM | 2437 | O | SER | A | 836 | −3.754 | −11.901 | 13.759 | 1.00 | 14.82 |
| ATOM | 2438 | N | GLU | A | 837 | −5.596 | −13.123 | 13.334 | 1.00 | 16.29 |
| ATOM | 2439 | CA | GLU | A | 837 | −5.423 | −13.139 | 11.886 | 1.00 | 16.40 |
| ATOM | 2440 | CB | GLU | A | 837 | −6.578 | −13.922 | 11.244 | 1.00 | 19.29 |
| ATOM | 2441 | CG | GLU | A | 837 | −6.524 | −14.017 | 9.722 | 1.00 | 30.56 |
| ATOM | 2442 | CD | GLU | A | 837 | −5.259 | −14.685 | 9.213 | 1.00 | 40.78 |
| ATOM | 2443 | OE1 | GLU | A | 837 | −4.925 | −15.785 | 9.704 | 1.00 | 33.63 |
| ATOM | 2444 | OE2 | GLU | A | 837 | −4.603 | −14.112 | 8.316 | 1.00 | 45.55 |
| ATOM | 2445 | C | GLU | A | 837 | −5.324 | −11.749 | 11.256 | 1.00 | 14.00 |
| ATOM | 2446 | O | GLU | A | 837 | −4.672 | −11.574 | 10.227 | 1.00 | 16.56 |
| ATOM | 2447 | N | ASP | A | 838 | −5.967 | −10.762 | 11.871 | 1.00 | 14.38 |
| ATOM | 2448 | CA | ASP | A | 838 | −5.938 | −9.402 | 11.349 | 1.00 | 12.78 |
| ATOM | 2449 | CB | ASP | A | 838 | −6.990 | −8.547 | 12.060 | 1.00 | 12.80 |
| ATOM | 2450 | CG | ASP | A | 838 | −8.410 | −8.929 | 11.669 | 1.00 | 31.66 |
| ATOM | 2451 | OD1 | ASP | A | 838 | −8.770 | −8.750 | 10.487 | 1.00 | 25.15 |
| ATOM | 2452 | OD2 | ASP | A | 838 | −9.162 | −9.414 | 12.540 | 1.00 | 29.94 |
| ATOM | 2453 | C | ASP | A | 838 | −4.558 | −8.751 | 11.468 | 1.00 | 11.81 |
| ATOM | 2454 | O | ASP | A | 838 | −4.355 | −7.625 | 11.017 | 1.00 | 12.87 |
| ATOM | 2455 | N | CYS | A | 839 | −3.610 | −9.456 | 12.080 | 1.00 | 14.34 |
| ATOM | 2456 | CA | CYS | A | 839 | −2.254 | −8.929 | 12.203 | 1.00 | 14.23 |
| ATOM | 2457 | CB | CYS | A | 839 | −1.619 | −9.354 | 13.533 | 1.00 | 12.27 |
| ATOM | 2458 | SG | CYS | A | 839 | −2.115 | −8.349 | 14.969 | 1.00 | 12.53 |
| ATOM | 2459 | C | CYS | A | 839 | −1.412 | −9.440 | 11.038 | 1.00 | 15.74 |
| ATOM | 2460 | O | CYS | A | 839 | −0.222 | −9.142 | 10.941 | 1.00 | 13.52 |
| ATOM | 2461 | N | PHE | A | 840 | −2.044 | −10.209 | 10.152 | 1.00 | 14.15 |
| ATOM | 2462 | CA | PHE | A | 840 | −1.364 | −10.765 | 8.983 | 1.00 | 14.85 |
| ATOM | 2463 | CB | PHE | A | 840 | −2.370 | −11.451 | 8.052 | 1.00 | 18.01 |
| ATOM | 2464 | CG | PHE | A | 840 | −1.760 | −11.926 | 6.764 | 1.00 | 16.92 |
| ATOM | 2465 | CD1 | PHE | A | 840 | −0.886 | −13.001 | 6.751 | 1.00 | 13.52 |
| ATOM | 2466 | CD2 | PHE | A | 840 | −2.010 | −11.257 | 5.575 | 1.00 | 19.38 |
| ATOM | 2467 | CE1 | PHE | A | 840 | −0.269 | −13.398 | 5.575 | 1.00 | 23.30 |
| ATOM | 2468 | CE2 | PHE | A | 840 | −1.397 | −11.649 | 4.396 | 1.00 | 23.75 |
| ATOM | 2469 | CZ | PHE | A | 840 | −0.525 | −12.720 | 4.397 | 1.00 | 22.76 |
| ATOM | 2470 | C | PHE | A | 840 | −0.541 | −9.768 | 8.155 | 1.00 | 13.70 |
| ATOM | 2471 | O | PHE | A | 840 | 0.546 | −10.105 | 7.678 | 1.00 | 13.33 |
| ATOM | 2472 | N | PRO | A | 841 | −1.049 | −8.537 | 7.958 | 1.00 | 12.97 |
| ATOM | 2473 | CD | PRO | A | 841 | −2.369 | −8.002 | 8.339 | 1.00 | 14.47 |
| ATOM | 2474 | CA | PRO | A | 841 | −0.294 | −7.557 | 7.168 | 1.00 | 12.84 |
| ATOM | 2475 | CB | PRO | A | 841 | −1.127 | −6.287 | 7.312 | 1.00 | 14.37 |
| ATOM | 2476 | CG | PRO | A | 841 | −2.533 | −6.826 | 7.391 | 1.00 | 14.58 |
| ATOM | 2477 | C | PRO | A | 841 | 1.151 | −7.367 | 7.630 | 1.00 | 14.54 |
| ATOM | 2478 | O | PRO | A | 841 | 2.034 | −7.065 | 6.826 | 1.00 | 13.75 |
| ATOM | 2479 | N | LEU | A | 842 | 1.393 | −7.530 | 8.926 | 1.00 | 11.66 |
| ATOM | 2480 | CA | LEU | A | 842 | 2.747 | −7.384 | 9.444 | 1.00 | 12.35 |
| ATOM | 2481 | CB | LEU | A | 842 | 2.732 | −7.430 | 10.977 | 1.00 | 11.85 |
| ATOM | 2482 | CG | LEU | A | 842 | 2.039 | −6.228 | 11.624 | 1.00 | 12.78 |
| ATOM | 2483 | CD1 | LEU | A | 842 | 1.900 | −6.434 | 13.131 | 1.00 | 14.23 |
| ATOM | 2484 | CD2 | LEU | A | 842 | 2.837 | −4.975 | 11.321 | 1.00 | 14.25 |
| ATOM | 2485 | C | LEU | A | 842 | 3.613 | −8.513 | 8.880 | 1.00 | 13.95 |
| ATOM | 2486 | O | LEU | A | 842 | 4.741 | −8.290 | 8.444 | 1.00 | 13.84 |
| ATOM | 2487 | N | LEU | A | 843 | 3.067 | −9.727 | 8.882 | 1.00 | 11.90 |
| ATOM | 2488 | CA | LEU | A | 843 | 3.779 | −10.889 | 8.359 | 1.00 | 11.64 |
| ATOM | 2489 | CB | LEU | A | 843 | 2.995 | −12.161 | 8.670 | 1.00 | 10.07 |
| ATOM | 2490 | CG | LEU | A | 843 | 3.532 | −13.468 | 8.084 | 1.00 | 9.50 |
| ATOM | 2491 | CD1 | LEU | A | 843 | 4.940 | −13.735 | 8.588 | 1.00 | 13.64 |
| ATOM | 2492 | CD2 | LEU | A | 843 | 2.597 | −14.608 | 8.466 | 1.00 | 12.37 |
| ATOM | 2493 | C | LEU | A | 843 | 3.971 | −10.751 | 6.847 | 1.00 | 11.74 |
| ATOM | 2494 | O | LEU | A | 843 | 5.054 | −11.003 | 6.319 | 1.00 | 13.14 |
| ATOM | 2495 | N | ASP | A | 844 | 2.909 | −10.342 | 6.160 | 1.00 | 13.31 |
| ATOM | 2496 | CA | ASP | A | 844 | 2.943 | −10.154 | 4.711 | 1.00 | 14.45 |
| ATOM | 2497 | CB | ASP | A | 844 | 1.573 | −9.639 | 4.245 | 1.00 | 18.19 |
| ATOM | 2498 | CG | ASP | A | 844 | 1.354 | −9.771 | 2.739 | 1.00 | 13.51 |
| ATOM | 2499 | OD1 | ASP | A | 844 | 2.141 | −10.459 | 2.057 | 1.00 | 18.86 |
| ATOM | 2500 | OD2 | ASP | A | 844 | 0.367 | −9.187 | 2.243 | 1.00 | 17.91 |
| ATOM | 2501 | C | ASP | A | 844 | 4.053 | −9.153 | 4.366 | 1.00 | 13.87 |
| ATOM | 2502 | O | ASP | A | 844 | 4.854 | −9.386 | 3.460 | 1.00 | 13.07 |
| ATOM | 2503 | N | GLY | A | 845 | 4.100 | −8.046 | 5.104 | 1.00 | 12.67 |
| ATOM | 2504 | CA | GLY | A | 845 | 5.115 | −7.035 | 4.862 | 1.00 | 11.69 |
| ATOM | 2505 | C | GLY | A | 845 | 6.529 | −7.540 | 5.097 | 1.00 | 11.05 |
| ATOM | 2506 | O | GLY | A | 845 | 7.447 | −7.240 | 4.330 | 1.00 | 12.51 |
| ATOM | 2507 | N | CYS | A | 846 | 6.704 | −8.314 | 6.161 | 1.00 | 10.28 |
| ATOM | 2508 | CA | CYS | A | 846 | 8.005 | −8.882 | 6.507 | 1.00 | 10.93 |
| ATOM | 2509 | CB | CYS | A | 846 | 7.889 | −9.643 | 7.831 | 1.00 | 14.58 |
| ATOM | 2510 | SG | CYS | A | 846 | 9.443 | −10.273 | 8.485 | 1.00 | 12.82 |
| ATOM | 2511 | C | CYS | A | 846 | 8.497 | −9.821 | 5.398 | 1.00 | 11.92 |
| ATOM | 2512 | O | CYS | A | 846 | 9.661 | −9.762 | 4.985 | 1.00 | 11.24 |
| ATOM | 2513 | N | ARG | A | 847 | 7.605 | −10.683 | 4.914 | 1.00 | 12.77 |
| ATOM | 2514 | CA | ARG | A | 847 | 7.953 | −11.618 | 3.853 | 1.00 | 13.72 |
| ATOM | 2515 | CB | ARG | A | 847 | 6.762 | −12.527 | 3.535 | 1.00 | 11.63 |
| ATOM | 2516 | CG | ARG | A | 847 | 6.457 | −13.545 | 4.619 | 1.00 | 13.10 |
| ATOM | 2517 | CD | ARG | A | 847 | 5.180 | −14.308 | 4.316 | 1.00 | 17.35 |
| ATOM | 2518 | NE | ARG | A | 847 | 4.978 | −15.402 | 5.262 | 1.00 | 19.23 |
| ATOM | 2519 | CZ | ARG | A | 847 | 3.840 | −16.077 | 5.389 | 1.00 | 21.47 |
| ATOM | 2520 | NH1 | ARG | A | 847 | 2.796 | −15.769 | 4.632 | 1.00 | 24.29 |
| ATOM | 2521 | NH2 | ARG | A | 847 | 3.750 | −17.059 | 6.275 | 1.00 | 27.21 |
| ATOM | 2522 | C | ARG | A | 847 | 8.371 | −10.870 | 2.592 | 1.00 | 13.53 |
| ATOM | 2523 | O | ARG | A | 847 | 9.320 | −11.265 | 1.906 | 1.00 | 13.20 |
| ATOM | 2524 | N | LYS | A | 848 | 7.659 | −9.793 | 2.283 | 1.00 | 12.35 |
| ATOM | 2525 | CA | LYS | A | 848 | 7.977 | −9.007 | 1.099 | 1.00 | 14.65 |
| ATOM | 2526 | CB | LYS | A | 848 | 6.925 | −7.915 | 0.880 | 1.00 | 16.22 |
| ATOM | 2527 | CG | LYS | A | 848 | 5.567 | −8.447 | 0.413 | 1.00 | 14.77 |
| ATOM | 2528 | CD | LYS | A | 848 | 4.579 | −7.308 | 0.195 | 1.00 | 19.65 |
| ATOM | 2529 | CE | LYS | A | 848 | 3.210 | −7.817 | −0.206 | 1.00 | 19.42 |
| ATOM | 2530 | NZ | LYS | A | 848 | 2.214 | −6.709 | −0.212 | 1.00 | 23.11 |
| ATOM | 2531 | C | LYS | A | 848 | 9.364 | −8.392 | 1.229 | 1.00 | 14.80 |
| ATOM | 2532 | O | LYS | A | 848 | 10.112 | −8.319 | 0.253 | 1.00 | 13.12 |
| ATOM | 2533 | N | ASN | A | 849 | 9.713 | −7.953 | 2.435 | 1.00 | 13.42 |
| ATOM | 2534 | CA | ASN | A | 849 | 11.034 | −7.369 | 2.648 | 1.00 | 13.07 |
| ATOM | 2535 | CB | ASN | A | 849 | 11.086 | −6.646 | 3.995 | 1.00 | 13.67 |
| ATOM | 2536 | CG | ASN | A | 849 | 10.412 | −5.288 | 3.950 | 1.00 | 17.85 |
| ATOM | 2537 | OD1 | ASN | A | 849 | 9.863 | −4.821 | 4.949 | 1.00 | 16.87 |
| ATOM | 2538 | ND2 | ASN | A | 849 | 10.461 | −4.638 | 2.790 | 1.00 | 13.21 |
| ATOM | 2539 | C | ASN | A | 849 | 12.122 | −8.434 | 2.568 | 1.00 | 12.00 |
| ATOM | 2540 | O | ASN | A | 849 | 13.252 | −8.148 | 2.165 | 1.00 | 11.88 |
| ATOM | 2541 | N | ARG | A | 850 | 11.797 | −9.667 | 2.945 | 1.00 | 11.53 |
| ATOM | 2542 | CA | ARG | A | 850 | 12.793 | −10.729 | 2.860 | 1.00 | 11.98 |
| ATOM | 2543 | CB | ARG | A | 850 | 12.285 | −12.037 | 3.473 | 1.00 | 14.13 |
| ATOM | 2544 | CG | ARG | A | 850 | 13.373 | −13.110 | 3.508 | 1.00 | 13.55 |
| ATOM | 2545 | CD | ARG | A | 850 | 12.838 | −14.511 | 3.781 | 1.00 | 18.02 |
| ATOM | 2546 | NE | ARG | A | 850 | 13.930 | −15.457 | 4.001 | 1.00 | 16.95 |
| ATOM | 2547 | CZ | ARG | A | 850 | 14.743 | −15.919 | 3.052 | 1.00 | 14.40 |
| ATOM | 2548 | NH1 | ARG | A | 850 | 14.595 | −15.533 | 1.790 | 1.00 | 16.03 |
| ATOM | 2549 | NH2 | ARG | A | 850 | 15.727 | −16.754 | 3.373 | 1.00 | 12.51 |
| ATOM | 2550 | C | ARG | A | 850 | 13.138 | −10.972 | 1.399 | 1.00 | 13.81 |
| ATOM | 2551 | O | ARG | A | 850 | 14.304 | −11.151 | 1.054 | 1.00 | 15.14 |
| ATOM | 2552 | N | GLN | A | 851 | 12.118 | −10.985 | 0.542 | 1.00 | 16.03 |
| ATOM | 2553 | CA | GLN | A | 851 | 12.317 | −11.193 | −0.892 | 1.00 | 15.42 |
| ATOM | 2554 | CB | GLN | A | 851 | 10.969 | −11.156 | −1.622 | 1.00 | 17.30 |
| ATOM | 2555 | CG | GLN | A | 851 | 11.039 | −11.399 | −3.127 | 1.00 | 30.46 |
| ATOM | 2556 | CD | GLN | A | 851 | 11.475 | −10.174 | −3.912 | 1.00 | 34.81 |
| ATOM | 2557 | OE1 | GLN | A | 851 | 10.862 | −9.110 | −3.814 | 1.00 | 37.41 |
| ATOM | 2558 | NE2 | GLN | A | 851 | 12.534 | −10.320 | −4.704 | 1.00 | 40.89 |
| ATOM | 2559 | C | GLN | A | 851 | 13.229 | −10.109 | −1.436 | 1.00 | 16.49 |
| ATOM | 2560 | O | GLN | A | 851 | 14.151 | −10.371 | −2.208 | 1.00 | 15.70 |
| ATOM | 2561 | N | LYS | A | 852 | 12.953 | −8.867 | −1.014 | 1.00 | 15.86 |
| ATOM | 2562 | CA | LYS | A | 852 | 13.705 | −7.685 | −1.428 | 1.00 | 16.63 |
| ATOM | 2563 | CB | LYS | A | 852 | 13.065 | −6.405 | −0.838 | 1.00 | 13.58 |
| ATOM | 2564 | CG | LYS | A | 852 | 11.802 | −6.008 | −1.561 | 1.00 | 18.37 |
| ATOM | 2565 | CD | LYS | A | 852 | 12.183 | −5.582 | −2.958 | 1.00 | 31.95 |
| ATOM | 2566 | CE | LYS | A | 852 | 11.002 | −5.087 | −3.751 | 1.00 | 37.83 |
| ATOM | 2567 | NZ | LYS | A | 852 | 11.421 | −4.599 | −5.099 | 1.00 | 41.92 |
| ATOM | 2568 | C | LYS | A | 852 | 15.175 | −7.754 | −1.037 | 1.00 | 15.46 |
| ATOM | 2569 | O | LYS | A | 852 | 16.055 | −7.542 | −1.874 | 1.00 | 14.58 |
| ATOM | 2570 | N | TRP | A | 853 | 15.446 | −8.063 | 0.225 | 1.00 | 12.99 |
| ATOM | 2571 | CA | TRP | A | 853 | 16.821 | −8.168 | 0.705 | 1.00 | 12.72 |
| ATOM | 2572 | CB | TRP | A | 853 | 16.843 | −8.299 | 2.228 | 1.00 | 11.94 |
| ATOM | 2573 | CG | TRP | A | 853 | 16.640 | −7.011 | 2.957 | 1.00 | 11.40 |
| ATOM | 2574 | CD2 | TRP | A | 853 | 17.485 | −5.864 | 2.913 | 1.00 | 14.74 |
| ATOM | 2575 | CE2 | TRP | A | 853 | 16.929 | −4.888 | 3.763 | 1.00 | 12.41 |
| ATOM | 2576 | CE3 | TRP | A | 853 | 18.675 | −5.554 | 2.236 | 1.00 | 10.90 |
| ATOM | 2577 | CD1 | TRP | A | 853 | 15.618 | −6.701 | 3.813 | 1.00 | 11.02 |
| ATOM | 2578 | NE1 | TRP | A | 853 | 15.781 | −5.429 | 4.303 | 1.00 | 14.15 |
| ATOM | 2579 | CZ2 | TRP | A | 853 | 17.502 | −3.638 | 3.958 | 1.00 | 12.03 |
| ATOM | 2580 | CZ3 | TRP | A | 853 | 19.246 | −4.312 | 2.427 | 1.00 | 13.77 |
| ATOM | 2581 | CH2 | TRP | A | 853 | 18.663 | −3.370 | 3.280 | 1.00 | 16.08 |
| ATOM | 2582 | C | TRP | A | 853 | 17.543 | −9.357 | 0.074 | 1.00 | 13.55 |
| ATOM | 2583 | O | TRP | A | 853 | 18.722 | −9.265 | −0.282 | 1.00 | 13.19 |
| ATOM | 2584 | N | GLN | A | 854 | 16.836 | −10.472 | −0.062 | 1.00 | 12.06 |
| ATOM | 2585 | CA | GLN | A | 854 | 17.427 | −11.667 | −0.657 | 1.00 | 13.49 |
| ATOM | 2586 | CB | GLN | A | 854 | 16.426 | −12.822 | −0.630 | 1.00 | 13.86 |
| ATOM | 2587 | CG | GLN | A | 854 | 16.934 | −14.128 | −1.242 | 1.00 | 15.91 |
| ATOM | 2588 | CD | GLN | A | 854 | 18.267 | −14.585 | −0.665 | 1.00 | 23.66 |
| ATOM | 2589 | OE1 | GLN | A | 854 | 18.544 | −14.404 | 0.520 | 1.00 | 20.44 |
| ATOM | 2590 | NE2 | GLN | A | 854 | 19.091 | −15.199 | −1.504 | 1.00 | 26.84 |
| ATOM | 2591 | C | GLN | A | 854 | 17.869 | −11.400 | −2.094 | 1.00 | 13.14 |
| ATOM | 2592 | O | GLN | A | 854 | 18.920 | −11.880 | −2.524 | 1.00 | 15.55 |
| ATOM | 2593 | N | ALA | A | 855 | 17.069 | −10.635 | −2.832 | 1.00 | 14.63 |
| ATOM | 2594 | CA | ALA | A | 855 | 17.408 | −10.318 | −4.219 | 1.00 | 15.08 |
| ATOM | 2595 | CB | ALA | A | 855 | 16.264 | −9.563 | −4.884 | 1.00 | 17.06 |
| ATOM | 2596 | C | ALA | A | 855 | 18.697 | −9.504 | −4.303 | 1.00 | 16.23 |
| ATOM | 2597 | O | ALA | A | 855 | 19.439 | −9.611 | −5.278 | 1.00 | 17.06 |
| ATOM | 2598 | N | LEU | A | 856 | 18.959 | −8.689 | −3.287 | 1.00 | 15.11 |
| ATOM | 2599 | CA | LEU | A | 856 | 20.178 | −7.889 | −3.261 | 1.00 | 17.23 |
| ATOM | 2600 | CB | LEU | A | 856 | 20.031 | −6.715 | −2.284 | 1.00 | 11.53 |
| ATOM | 2601 | CG | LEU | A | 856 | 18.968 | −5.676 | −2.643 | 1.00 | 15.60 |
| ATOM | 2602 | CD1 | LEU | A | 856 | 18.996 | −4.549 | −1.634 | 1.00 | 19.73 |
| ATOM | 2603 | CD2 | LEU | A | 856 | 19.226 | −5.145 | −4.047 | 1.00 | 12.95 |
| ATOM | 2604 | C | LEU | A | 856 | 21.362 | −8.754 | −2.835 | 1.00 | 17.35 |
| ATOM | 2605 | O | LEU | A | 856 | 22.478 | −8.601 | −3.337 | 1.00 | 17.30 |
| ATOM | 2606 | N | ALA | A | 857 | 21.109 | −9.674 | −1.911 | 1.00 | 16.52 |
| ATOM | 2607 | CA | ALA | A | 857 | 22.156 | −10.545 | −1.396 | 1.00 | 17.82 |
| ATOM | 2608 | CB | ALA | A | 857 | 21.708 | −11.155 | −0.079 | 1.00 | 15.78 |
| ATOM | 2609 | C | ALA | A | 857 | 22.616 | −11.651 | −2.339 | 1.00 | 19.95 |
| ATOM | 2610 | O | ALA | A | 857 | 23.781 | −12.048 | −2.305 | 1.00 | 21.30 |
| ATOM | 2611 | N | GLU | A | 858 | 21.715 | −12.144 | −3.181 | 1.00 | 19.75 |
| ATOM | 2612 | CA | GLU | A | 858 | 22.059 | −13.233 | −4.091 | 1.00 | 23.14 |
| ATOM | 2613 | CB | GLU | A | 858 | 20.798 | −14.008 | −4.475 | 1.00 | 28.23 |
| ATOM | 2614 | CG | GLU | A | 858 | 19.735 | −13.155 | −5.139 | 1.00 | 35.46 |
| ATOM | 2615 | CD | GLU | A | 858 | 18.516 | −13.957 | −5.548 | 1.00 | 41.30 |
| ATOM | 2616 | OE1 | GLU | A | 858 | 17.928 | −14.635 | −4.679 | 1.00 | 46.06 |
| ATOM | 2617 | OE2 | GLU | A | 858 | 18.144 | −13.905 | −6.739 | 1.00 | 53.40 |
| ATOM | 2618 | C | GLU | A | 858 | 22.773 | −12.775 | −5.356 | 1.00 | 24.66 |
| ATOM | 2619 | O | GLU | A | 858 | 22.769 | −11.561 | −5.640 | 1.00 | 27.06 |
| ATOM | 2620 | OXT | GLU | A | 858 | 23.319 | −13.654 | −6.053 | 1.00 | 33.90 |
| TER | 2621 | | GLU | A | 858 | | | | | |
| ATOM | 2622 | O | HOH | W | 1 | 14.659 | 12.373 | 18.930 | 1.00 | 11.90 |
| ATOM | 2623 | O | HOH | W | 2 | 9.029 | 0.616 | 15.467 | 1.00 | 15.60 |
| ATOM | 2624 | O | HOH | W | 3 | 17.339 | −0.310 | 24.269 | 1.00 | 9.87 |
| ATOM | 2625 | O | HOH | W | 4 | 17.787 | 8.492 | 22.312 | 1.00 | 9.91 |
| ATOM | 2626 | O | HOH | W | 5 | 22.322 | 0.101 | 41.405 | 1.00 | 10.58 |
| ATOM | 2627 | O | HOH | W | 6 | 7.037 | −4.929 | 2.938 | 1.00 | 15.52 |
| ATOM | 2628 | O | HOH | W | 7 | 24.844 | 0.942 | 17.890 | 1.00 | 12.19 |
| ATOM | 2629 | O | HOH | W | 8 | 17.812 | −5.285 | 21.940 | 1.00 | 13.09 |
| ATOM | 2630 | O | HOH | W | 9 | 19.355 | −1.244 | 21.749 | 1.00 | 11.09 |
| ATOM | 2631 | O | HOH | W | 10 | 11.998 | −15.929 | 13.123 | 1.00 | 15.08 |
| ATOM | 2632 | O | HOH | W | 11 | 17.595 | 6.868 | 5.858 | 1.00 | 17.40 |
| ATOM | 2633 | O | HOH | W | 12 | 20.522 | −8.124 | 28.490 | 1.00 | 16.30 |
| ATOM | 2634 | O | HOH | W | 13 | 7.122 | 8.426 | 30.706 | 1.00 | 12.74 |
| ATOM | 2635 | O | HOH | W | 14 | 14.281 | −0.736 | 24.882 | 1.00 | 10.20 |
| ATOM | 2636 | O | HOH | W | 15 | 27.778 | −4.812 | 7.714 | 1.00 | 14.34 |
| ATOM | 2637 | O | HOH | W | 16 | 45.697 | −5.856 | 19.373 | 1.00 | 13.79 |
| ATOM | 2638 | O | HOH | W | 17 | 32.028 | 0.851 | 11.859 | 1.00 | 12.49 |
| ATOM | 2639 | O | HOH | W | 18 | 12.716 | −7.833 | 22.260 | 1.00 | 11.62 |
| ATOM | 2640 | O | HOH | W | 19 | 20.770 | −7.019 | 24.953 | 1.00 | 13.66 |
| ATOM | 2641 | O | HOH | W | 20 | 9.679 | −8.387 | 30.457 | 1.00 | 18.04 |
| ATOM | 2642 | O | HOH | W | 21 | 20.996 | −1.566 | 19.584 | 1.00 | 15.27 |
| ATOM | 2643 | O | HOH | W | 22 | 5.367 | −8.570 | 29.548 | 1.00 | 17.36 |
| ATOM | 2644 | O | HOH | W | 23 | 25.138 | −9.337 | 1.725 | 1.00 | 17.63 |
| ATOM | 2645 | O | HOH | W | 24 | −9.020 | −0.116 | 43.708 | 1.00 | 15.91 |
| ATOM | 2646 | O | HOH | W | 25 | 22.657 | −6.174 | 29.194 | 1.00 | 17.18 |
| ATOM | 2647 | O | HOH | W | 26 | −4.280 | 19.557 | 21.192 | 1.00 | 19.85 |
| ATOM | 2648 | O | HOH | W | 27 | 14.289 | 6.604 | 13.852 | 1.00 | 14.85 |
| ATOM | 2649 | O | HOH | W | 28 | 11.411 | −1.302 | 13.935 | 1.00 | 12.80 |
| ATOM | 2650 | O | HOH | W | 29 | 11.700 | 13.882 | 15.200 | 1.00 | 14.97 |
| ATOM | 2651 | O | HOH | W | 30 | 18.410 | 10.076 | 12.196 | 1.00 | 19.59 |
| ATOM | 2652 | O | HOH | W | 31 | 26.709 | 7.571 | 23.378 | 1.00 | 17.44 |
| ATOM | 2653 | O | HOH | W | 32 | 25.420 | 5.805 | 17.773 | 1.00 | 17.40 |
| ATOM | 2654 | O | HOH | W | 33 | 11.087 | 10.589 | 10.070 | 1.00 | 17.81 |
| ATOM | 2655 | O | HOH | W | 34 | 11.500 | −19.954 | 26.231 | 1.00 | 16.07 |
| ATOM | 2656 | O | HOH | W | 35 | −5.204 | −5.042 | 34.106 | 1.00 | 17.24 |
| ATOM | 2657 | O | HOH | W | 36 | 1.941 | 9.940 | 36.428 | 1.00 | 13.95 |
| ATOM | 2658 | O | HOH | W | 37 | 23.916 | 7.238 | 15.924 | 1.00 | 14.00 |
| ATOM | 2659 | O | HOH | W | 38 | 4.522 | 21.473 | 19.911 | 1.00 | 20.60 |
| ATOM | 2660 | O | HOH | W | 39 | 7.654 | 32.374 | 24.586 | 1.00 | 19.77 |
| ATOM | 2661 | O | HOH | W | 40 | −11.105 | 8.617 | 12.732 | 1.00 | 20.19 |
| ATOM | 2662 | O | HOH | W | 41 | 14.266 | 14.289 | 20.855 | 1.00 | 16.50 |
| ATOM | 2663 | O | HOH | W | 42 | 27.797 | −9.140 | 13.072 | 1.00 | 17.28 |
| ATOM | 2664 | O | HOH | W | 43 | 13.361 | −5.781 | 20.636 | 1.00 | 17.62 |
| ATOM | 2665 | O | HOH | W | 44 | 9.439 | 17.952 | 22.920 | 1.00 | 20.34 |
| ATOM | 2666 | O | HOH | W | 45 | 13.506 | −19.339 | 23.851 | 1.00 | 17.51 |
| ATOM | 2667 | O | HOH | W | 46 | −9.608 | −2.484 | 44.794 | 1.00 | 18.03 |
| ATOM | 2668 | O | HOH | W | 47 | 19.179 | −3.517 | 23.479 | 1.00 | 14.63 |
| ATOM | 2669 | O | HOH | W | 48 | 20.825 | −1.771 | 7.357 | 1.00 | 15.56 |
| ATOM | 2670 | O | HOH | W | 49 | 3.953 | −2.401 | 8.103 | 1.00 | 15.98 |
| ATOM | 2671 | O | HOH | W | 50 | 5.164 | 2.828 | 12.389 | 1.00 | 17.57 |
| ATOM | 2672 | O | HOH | W | 51 | 22.475 | −6.004 | 8.390 | 1.00 | 20.31 |
| ATOM | 2673 | O | HOH | W | 52 | −1.505 | −7.713 | 3.922 | 1.00 | 20.93 |
| ATOM | 2674 | O | HOH | W | 53 | 16.030 | 13.344 | 15.066 | 1.00 | 22.86 |
| ATOM | 2675 | O | HOH | W | 54 | 3.120 | 33.114 | 17.327 | 1.00 | 18.81 |
| ATOM | 2676 | O | HOH | W | 55 | 6.517 | 19.676 | 19.112 | 1.00 | 20.43 |
| ATOM | 2677 | O | HOH | W | 56 | 27.812 | 5.941 | 11.269 | 1.00 | 19.39 |
| ATOM | 2678 | O | HOH | W | 57 | 14.704 | 9.138 | 12.638 | 1.00 | 17.37 |
| ATOM | 2679 | O | HOH | W | 58 | 10.813 | −19.129 | 28.734 | 1.00 | 23.50 |
| ATOM | 2680 | O | HOH | W | 59 | 9.603 | −13.958 | 1.403 | 1.00 | 20.05 |
| ATOM | 2681 | O | HOH | W | 60 | 46.209 | −3.550 | 17.835 | 1.00 | 18.22 |
| ATOM | 2682 | O | HOH | W | 61 | 24.760 | −2.396 | 10.006 | 1.00 | 20.80 |
| ATOM | 2683 | O | HOH | W | 62 | 11.077 | 3.087 | 12.585 | 1.00 | 21.05 |
| ATOM | 2684 | O | HOH | W | 63 | 10.534 | −10.726 | 28.998 | 1.00 | 21.18 |
| ATOM | 2685 | O | HOH | W | 64 | −0.961 | −11.064 | 33.440 | 1.00 | 18.63 |
| ATOM | 2686 | O | HOH | W | 65 | 20.890 | 0.577 | 15.388 | 1.00 | 19.77 |
| ATOM | 2687 | O | HOH | W | 66 | 21.685 | 13.866 | 20.281 | 1.00 | 24.54 |
| ATOM | 2688 | O | HOH | W | 67 | −5.012 | −3.878 | 8.180 | 1.00 | 21.24 |
| ATOM | 2689 | O | HOH | W | 68 | −10.443 | 9.371 | 15.194 | 1.00 | 19.03 |
| ATOM | 2690 | O | HOH | W | 69 | 26.635 | 8.578 | 39.152 | 1.00 | 19.48 |
| ATOM | 2691 | O | HOH | W | 70 | 21.837 | −4.044 | 23.094 | 1.00 | 14.46 |
| ATOM | 2692 | O | HOH | W | 71 | 18.979 | 0.534 | 3.275 | 1.00 | 19.32 |
| ATOM | 2693 | O | HOH | W | 72 | −7.687 | −4.529 | 35.484 | 1.00 | 19.76 |
| ATOM | 2694 | O | HOH | W | 73 | −4.441 | 9.972 | 35.483 | 1.00 | 21.75 |
| ATOM | 2695 | O | HOH | W | 74 | 22.147 | 0.611 | 18.233 | 1.00 | 21.06 |
| ATOM | 2696 | O | HOH | W | 75 | 25.625 | 7.694 | 19.834 | 1.00 | 17.79 |
| ATOM | 2697 | O | HOH | W | 76 | −9.716 | 14.941 | 11.027 | 1.00 | 20.22 |
| ATOM | 2698 | O | HOH | W | 77 | 14.015 | −1.710 | 16.866 | 1.00 | 19.03 |
| ATOM | 2699 | O | HOH | W | 78 | 21.861 | −3.953 | 20.354 | 1.00 | 17.68 |
| ATOM | 2700 | O | HOH | W | 79 | 21.854 | −11.471 | 10.659 | 1.00 | 21.05 |
| ATOM | 2701 | O | HOH | W | 80 | 15.795 | −5.944 | −4.295 | 1.00 | 17.34 |
| ATOM | 2702 | O | HOH | W | 81 | 26.427 | −5.333 | 20.748 | 1.00 | 28.65 |
| ATOM | 2703 | O | HOH | W | 82 | 0.806 | 16.637 | 36.547 | 1.00 | 22.11 |
| ATOM | 2704 | O | HOH | W | 83 | 28.883 | 5.909 | 33.300 | 1.00 | 35.99 |
| ATOM | 2705 | O | HOH | W | 84 | 0.615 | −5.836 | 36.804 | 1.00 | 21.63 |
| ATOM | 2706 | O | HOH | W | 85 | 14.416 | −12.696 | −3.692 | 1.00 | 22.36 |
| ATOM | 2707 | O | HOH | W | 86 | −4.841 | 7.644 | 43.980 | 1.00 | 19.92 |
| ATOM | 2708 | O | HOH | W | 87 | −6.059 | 5.564 | 10.293 | 1.00 | 22.49 |
| ATOM | 2709 | O | HOH | W | 88 | 7.210 | 1.031 | 13.002 | 1.00 | 19.78 |
| ATOM | 2710 | O | HOH | W | 89 | −5.786 | −6.329 | 9.023 | 1.00 | 20.32 |
| ATOM | 2711 | O | HOH | W | 90 | 19.117 | 4.909 | 35.027 | 1.00 | 17.30 |
| ATOM | 2712 | O | HOH | W | 91 | 10.592 | 13.017 | 38.293 | 1.00 | 23.66 |
| ATOM | 2713 | O | HOH | W | 92 | −9.801 | 5.234 | 16.132 | 1.00 | 20.64 |
| ATOM | 2714 | O | HOH | W | 93 | −6.370 | −17.172 | 24.540 | 1.00 | 23.65 |
| ATOM | 2715 | O | HOH | W | 94 | −6.833 | 1.342 | 45.207 | 1.00 | 17.92 |
| ATOM | 2716 | O | HOH | W | 95 | 33.715 | −7.279 | 11.000 | 1.00 | 17.67 |
| ATOM | 2717 | O | HOH | W | 96 | 5.722 | 31.659 | 29.252 | 1.00 | 19.35 |
| ATOM | 2718 | O | HOH | W | 97 | 14.155 | −2.890 | 10.083 | 1.00 | 16.30 |
| ATOM | 2719 | O | HOH | W | 98 | 19.709 | −5.898 | 20.414 | 1.00 | 27.45 |
| ATOM | 2720 | O | HOH | W | 99 | 4.418 | −3.958 | 2.629 | 1.00 | 23.70 |
| ATOM | 2721 | O | HOH | W | 100 | 5.739 | 5.419 | 41.566 | 1.00 | 26.30 |
| ATOM | 2722 | O | HOH | W | 101 | 1.882 | −10.334 | 12.547 | 1.00 | 21.56 |
| ATOM | 2723 | O | HOH | W | 102 | 1.506 | 9.558 | 7.516 | 1.00 | 21.76 |
| ATOM | 2724 | O | HOH | W | 103 | 23.211 | 1.757 | 4.698 | 1.00 | 23.21 |
| ATOM | 2725 | O | HOH | W | 104 | 36.282 | 2.363 | 9.440 | 1.00 | 27.13 |
| ATOM | 2726 | O | HOH | W | 105 | 27.600 | −11.267 | 2.472 | 1.00 | 18.59 |
| ATOM | 2727 | O | HOH | W | 106 | 16.305 | 16.757 | 22.862 | 1.00 | 25.14 |
| ATOM | 2728 | O | HOH | W | 107 | 21.223 | −0.443 | 4.807 | 1.00 | 20.51 |
| ATOM | 2729 | O | HOH | W | 108 | 12.939 | 13.840 | 12.680 | 1.00 | 26.10 |
| ATOM | 2730 | O | HOH | W | 109 | 10.191 | −9.652 | 24.549 | 1.00 | 17.97 |
| ATOM | 2731 | O | HOH | W | 110 | 9.527 | −19.990 | 11.160 | 1.00 | 34.52 |
| ATOM | 2732 | O | HOH | W | 111 | 2.300 | −4.117 | 6.982 | 1.00 | 21.40 |
| ATOM | 2733 | O | HOH | W | 112 | 31.807 | 5.599 | 25.384 | 1.00 | 19.28 |
| ATOM | 2734 | O | HOH | W | 113 | 11.858 | 5.625 | 12.858 | 1.00 | 18.80 |
| ATOM | 2735 | O | HOH | W | 114 | 2.975 | 3.851 | 3.583 | 1.00 | 28.02 |
| ATOM | 2736 | O | HOH | W | 115 | 22.328 | −3.325 | 9.278 | 1.00 | 18.13 |
| ATOM | 2737 | O | HOH | W | 116 | 7.581 | 21.278 | 17.210 | 1.00 | 26.33 |
| ATOM | 2738 | O | HOH | W | 117 | −8.959 | 18.330 | 19.702 | 1.00 | 25.41 |
| ATOM | 2739 | O | HOH | W | 118 | −7.913 | −3.625 | 46.688 | 1.00 | 16.16 |
| ATOM | 2740 | O | HOH | W | 119 | 29.369 | −4.122 | 35.016 | 1.00 | 21.35 |
| ATOM | 2741 | O | HOH | W | 120 | −10.313 | 4.301 | 39.676 | 1.00 | 20.84 |
| ATOM | 2742 | O | HOH | W | 121 | 36.288 | −8.163 | 10.383 | 1.00 | 29.41 |
| ATOM | 2743 | O | HOH | W | 122 | −5.305 | 0.154 | 7.066 | 1.00 | 32.03 |
| ATOM | 2744 | O | HOH | W | 123 | 15.892 | 9.345 | 6.948 | 1.00 | 31.98 |
| ATOM | 2745 | O | HOH | W | 124 | −7.934 | −11.981 | 26.594 | 1.00 | 24.88 |
| ATOM | 2746 | O | HOH | W | 125 | −7.700 | −14.762 | 14.417 | 1.00 | 27.16 |
| ATOM | 2747 | O | HOH | W | 126 | 7.924 | 19.345 | 32.073 | 1.00 | 27.10 |
| ATOM | 2748 | O | HOH | W | 127 | 22.963 | −15.271 | −0.194 | 1.00 | 34.57 |
| ATOM | 2749 | O | HOH | W | 128 | 14.018 | 4.418 | 0.344 | 1.00 | 27.94 |
| ATOM | 2750 | O | HOH | W | 129 | 13.334 | 16.377 | 17.301 | 1.00 | 27.05 |
| ATOM | 2751 | O | HOH | W | 130 | 30.641 | −11.391 | 0.176 | 1.00 | 21.79 |
| ATOM | 2752 | O | HOH | W | 131 | −5.143 | −9.116 | 6.397 | 1.00 | 26.22 |
| ATOM | 2753 | O | HOH | W | 132 | 44.452 | 2.118 | 22.189 | 1.00 | 25.35 |
| ATOM | 2754 | O | HOH | W | 133 | −0.942 | −8.268 | 32.526 | 1.00 | 21.73 |
| ATOM | 2755 | O | HOH | W | 134 | 32.286 | 8.100 | 17.531 | 1.00 | 30.18 |
| ATOM | 2756 | O | HOH | W | 135 | 4.471 | −22.268 | 17.254 | 1.00 | 22.18 |
| ATOM | 2757 | O | HOH | W | 136 | 20.964 | −7.154 | 10.051 | 1.00 | 23.34 |
| ATOM | 2758 | O | HOH | W | 137 | −6.984 | 26.107 | 22.746 | 1.00 | 26.33 |
| ATOM | 2759 | O | HOH | W | 138 | 17.856 | 14.348 | 24.807 | 1.00 | 20.33 |
| ATOM | 2760 | O | HOH | W | 139 | 10.038 | −5.546 | 41.193 | 1.00 | 26.62 |
| ATOM | 2761 | O | HOH | W | 140 | 13.989 | 13.837 | 16.687 | 1.00 | 27.53 |
| ATOM | 2762 | O | HOH | W | 141 | 23.629 | −13.084 | 9.502 | 1.00 | 28.21 |
| ATOM | 2763 | O | HOH | W | 142 | 31.635 | 7.713 | 14.307 | 1.00 | 30.30 |
| ATOM | 2764 | O | HOH | W | 143 | 6.811 | −2.344 | −0.868 | 1.00 | 24.47 |
| ATOM | 2765 | O | HOH | W | 144 | 7.278 | −9.981 | 30.756 | 1.00 | 27.03 |
| ATOM | 2766 | O | HOH | W | 145 | −6.981 | 7.089 | 6.281 | 1.00 | 26.94 |
| ATOM | 2767 | O | HOH | W | 146 | −8.635 | 18.558 | 29.861 | 1.00 | 27.91 |
| ATOM | 2768 | O | HOH | W | 147 | 30.525 | −1.680 | 34.334 | 1.00 | 24.09 |
| ATOM | 2769 | O | HOH | W | 148 | 2.251 | −13.867 | 2.121 | 1.00 | 25.49 |
| ATOM | 2770 | O | HOH | W | 149 | −8.063 | −9.740 | 29.191 | 1.00 | 24.34 |
| ATOM | 2771 | O | HOH | W | 150 | −1.333 | 15.279 | 37.020 | 1.00 | 28.81 |
| ATOM | 2772 | O | HOH | W | 151 | 18.429 | 15.610 | 21.408 | 1.00 | 22.50 |
| ATOM | 2773 | O | HOH | W | 152 | 13.471 | −4.264 | 12.528 | 1.00 | 19.38 |
| ATOM | 2774 | O | HOH | W | 153 | 18.621 | −17.600 | 7.222 | 1.00 | 21.52 |
| ATOM | 2775 | O | HOH | W | 154 | 19.181 | −5.396 | 13.023 | 1.00 | 33.83 |
| ATOM | 2776 | O | HOH | W | 155 | −3.698 | −11.419 | 33.622 | 1.00 | 22.32 |
| ATOM | 2777 | O | HOH | W | 156 | 24.773 | −7.687 | 26.828 | 1.00 | 25.23 |
| ATOM | 2778 | O | HOH | W | 157 | 11.338 | 6.981 | 10.450 | 1.00 | 25.98 |
| ATOM | 2779 | O | HOH | W | 158 | 7.187 | 4.097 | 9.722 | 1.00 | 25.73 |
| ATOM | 2780 | O | HOH | W | 159 | 10.571 | 18.276 | 18.940 | 1.00 | 26.56 |
| ATOM | 2781 | O | HOH | W | 160 | 29.444 | 8.138 | 18.655 | 1.00 | 25.10 |
| ATOM | 2782 | O | HOH | W | 161 | −4.733 | −16.782 | 12.321 | 1.00 | 23.51 |
| ATOM | 2783 | O | HOH | W | 162 | 24.194 | 7.218 | 40.602 | 1.00 | 28.88 |
| ATOM | 2784 | O | HOH | W | 163 | 13.925 | −7.040 | −5.961 | 1.00 | 29.58 |
| ATOM | 2785 | O | HOH | W | 164 | −8.456 | 17.425 | 10.469 | 1.00 | 25.03 |
| ATOM | 2786 | O | HOH | W | 165 | 20.283 | 7.809 | 6.109 | 1.00 | 25.58 |
| ATOM | 2787 | O | HOH | W | 166 | 11.226 | 1.360 | 14.389 | 1.00 | 28.28 |
| ATOM | 2788 | O | HOH | W | 167 | 17.600 | 11.985 | 25.766 | 1.00 | 28.21 |
| ATOM | 2789 | O | HOH | W | 168 | −1.369 | 0.364 | 6.150 | 1.00 | 26.61 |
| ATOM | 2790 | O | HOH | W | 169 | 1.250 | 6.912 | 6.112 | 1.00 | 22.96 |
| ATOM | 2791 | O | HOH | W | 170 | 13.982 | 16.820 | 19.827 | 1.00 | 26.70 |
| ATOM | 2792 | O | HOH | W | 171 | 8.401 | 6.117 | 8.285 | 1.00 | 29.37 |
| ATOM | 2793 | O | HOH | W | 172 | 1.561 | 32.980 | 20.315 | 1.00 | 25.71 |
| ATOM | 2794 | O | HOH | W | 173 | 8.687 | −4.960 | 0.724 | 1.00 | 20.63 |
| ATOM | 2795 | O | HOH | W | 174 | 16.762 | −8.584 | 32.541 | 1.00 | 22.53 |
| ATOM | 2796 | O | HOH | W | 175 | −8.928 | 23.790 | 25.122 | 1.00 | 26.68 |
| ATOM | 2797 | O | HOH | W | 176 | 30.774 | −10.692 | 12.781 | 1.00 | 29.33 |
| ATOM | 2798 | O | HOH | W | 177 | −4.491 | −0.583 | 48.435 | 1.00 | 27.59 |
| ATOM | 2799 | O | HOH | W | 178 | −10.254 | 8.887 | 25.677 | 1.00 | 27.41 |
| ATOM | 2800 | O | HOH | W | 179 | 12.487 | −14.347 | 0.366 | 1.00 | 29.02 |
| ATOM | 2801 | O | HOH | W | 180 | −2.194 | −3.193 | 5.572 | 1.00 | 37.26 |
| ATOM | 2802 | O | HOH | W | 181 | 21.240 | −14.747 | 4.723 | 1.00 | 24.12 |
| ATOM | 2803 | O | HOH | W | 182 | 25.238 | 15.872 | 21.083 | 1.00 | 33.15 |
| ATOM | 2804 | O | HOH | W | 183 | 27.650 | 8.541 | 12.497 | 1.00 | 23.15 |
| ATOM | 2805 | O | HOH | W | 184 | 35.303 | 3.837 | 26.688 | 1.00 | 23.28 |
| ATOM | 2806 | O | HOH | W | 185 | −3.730 | 14.555 | 34.107 | 1.00 | 23.34 |
| ATOM | 2807 | O | HOH | W | 186 | −8.927 | −4.660 | 10.381 | 1.00 | 28.70 |
| ATOM | 2808 | O | HOH | W | 187 | 40.695 | −1.179 | 15.166 | 1.00 | 31.35 |
| ATOM | 2809 | O | HOH | W | 188 | −0.356 | −22.615 | 31.607 | 1.00 | 35.28 |
| ATOM | 2810 | O | HOH | W | 189 | 3.463 | 34.674 | 22.382 | 1.00 | 27.20 |
| ATOM | 2811 | O | HOH | W | 190 | 8.911 | 2.196 | 11.376 | 1.00 | 19.30 |
| ATOM | 2812 | O | HOH | W | 191 | −2.598 | −24.115 | 28.861 | 1.00 | 26.04 |
| ATOM | 2813 | O | HOH | W | 192 | 8.418 | −15.510 | 34.179 | 1.00 | 38.02 |
| ATOM | 2814 | O | HOH | W | 193 | 15.703 | −17.633 | −0.060 | 1.00 | 35.88 |
| ATOM | 2815 | O | HOH | W | 194 | 32.518 | 0.997 | 3.614 | 1.00 | 34.25 |
| ATOM | 2816 | O | HOH | W | 195 | 19.062 | −16.798 | 10.198 | 1.00 | 27.69 |
| ATOM | 2817 | O | HOH | W | 196 | 13.216 | 8.976 | 10.392 | 1.00 | 27.61 |
| ATOM | 2818 | O | HOH | W | 197 | 2.647 | −0.086 | 12.019 | 1.00 | 23.91 |
| ATOM | 2819 | O | HOH | W | 198 | −1.276 | −7.561 | 35.325 | 1.00 | 25.96 |
| ATOM | 2820 | O | HOH | W | 199 | 4.267 | −22.362 | 30.170 | 1.00 | 35.68 |
| ATOM | 2821 | O | HOH | W | 200 | 21.787 | 10.595 | 30.558 | 1.00 | 32.19 |
| ATOM | 2822 | O | HOH | W | 201 | 21.638 | 20.389 | 14.210 | 1.00 | 43.09 |
| ATOM | 2823 | O | HOH | W | 202 | 5.373 | −10.297 | 38.927 | 1.00 | 36.29 |
| ATOM | 2824 | O | HOH | W | 203 | 29.035 | −9.588 | 1.139 | 1.00 | 25.61 |
| ATOM | 2825 | O | HOH | W | 204 | 0.403 | −17.629 | 11.462 | 1.00 | 25.52 |
| ATOM | 2826 | O | HOH | W | 205 | 15.896 | −2.824 | 23.429 | 1.00 | 9.81 |
| ATOM | 2827 | O | HOH | W | 206 | 13.783 | −1.283 | 21.923 | 1.00 | 12.03 |
| ATOM | 2828 | O | HOH | W | 207 | 16.854 | −0.738 | 21.328 | 1.00 | 11.76 |
| ATOM | 2829 | O | HOH | W | 208 | 14.946 | −0.465 | 19.263 | 1.00 | 10.55 |
| ATOM | 2830 | O | HOH | W | 209 | −5.566 | −9.500 | 29.835 | 1.00 | 34.12 |
| ATOM | 2831 | O | HOH | W | 210 | 11.435 | 14.251 | 33.693 | 1.00 | 48.96 |
| ATOM | 2832 | O | HOH | W | 211 | 3.944 | 31.281 | 18.816 | 1.00 | 43.83 |
| ATOM | 2833 | O | HOH | W | 212 | 21.511 | 8.201 | 40.401 | 1.00 | 42.14 |
| ATOM | 2834 | O | HOH | W | 213 | 44.825 | 1.178 | 19.793 | 1.00 | 52.54 |
| ATOM | 2835 | O | HOH | W | 214 | 9.397 | 20.244 | 23.958 | 1.00 | 30.91 |
| ATOM | 2836 | O | HOH | W | 215 | 18.636 | −1.182 | 15.865 | 1.00 | 23.82 |
| ATOM | 2837 | O | HOH | W | 216 | 18.955 | −2.693 | 17.957 | 1.00 | 28.82 |
| ATOM | 2838 | O | HOH | W | 217 | 26.248 | −9.541 | 27.965 | 1.00 | 25.10 |
| ATOM | 2839 | O | HOH | W | 218 | 24.703 | −9.867 | 30.146 | 1.00 | 25.52 |
| ATOM | 2840 | O | HOH | W | 219 | 22.600 | −9.260 | 25.832 | 1.00 | 26.71 |
| ATOM | 2841 | O | HOH | W | 220 | 1.825 | 10.054 | 39.457 | 1.00 | 25.12 |
| ATOM | 2842 | O | HOH | W | 221 | 3.585 | 4.590 | 45.058 | 1.00 | 28.59 |
| ATOM | 2843 | O | HOH | W | 222 | 3.777 | −6.424 | 30.447 | 1.00 | 24.42 |
| TER | 2844 | | HOH | W | 222 | | | | | |
| ATOM | 2845 | ZN | ZN | B | 864 | 13.210 | 0.069 | 20.406 | 1.00 | 15.15 |
| ATOM | 2846 | MG | MG | B | 865 | 15.487 | −0.725 | 23.006 | 1.00 | 8.84 |
| TER | 2847 | | MG | B | 865 | | | | | |
| END | | | | | | | | | | |

References
Abrahams, J. P. & Leslie, A. (1996) Acta Crystallogr. D 52, 30-42.
Aravind, L. & Ponting, C. P. (1997) TIBS. 22, 458-459.
Briinger, A. et al. (1998), Acta Crystallogr. D 54, 905-921.
Butcher, R. W. & Sutherland, E. W. (1962) J. biol. Chem. 237, 1244-1250.
Charbonneau, H. (1990) in Cyclic Nucleotide Phosphodiesterases: Structure, Regulation and Drug Action (Beavo, J., and Houslay, M. D., eds) pp. 267-296, John Wiley & Sons, Inc., New York.
Charbonneau, H. et al., (1986) Proc. Natl. Acad. Sci. U.S.A., 83, 9308-9312.
Collaborative Computational Project Number 4 (1994) Acta Crystallogr. D 50, 760-763.
Corbin, J. D. & Francis, S. H. (1999) J. Biol. Chem. 274, 13729-13732.
de La Fortelle, E. & Bricogne, G. (1997) Methods Enzymol. 276, 472-494.
Francis, S. H. et al. (1994) J. Biol. Chem. 269, 22477-22480.
Goldberg, N. D. et al (1980) J. Biol. Chem. 255, 10344-10347.
Hendrickson, W. A. et al. (1989) Basic Life Sci. 51 (Synchrotron Radiat. Struct. Biol.), 317-324.
Jiang, H. et al. (1992) J. Biol. Chem. 267, 1015-1019.
Laskowski, R. A. et al. (1993) J. Appl. Crystallogr. 26, 283-291.
Lohman, S. M. et al. (1997) Trends in Biochem. Sci. 22, 307-312.
Martinez, S. et al. (2001) Poster presented at the American Crystallography Association annual meeting, Los Angeles, USA.
Matthews, B. W. (1968) J. Mol. Biol. 33, 491-497.
Nicholls, A. et al. (1993) J. Biophys, A166.
Otwinowski, Z. & Minor, W. (1997) Methods Enzymol. 276, 307-326.
Quanta98, 1998, version 98.1111; Molecular Simulations Inc., San Diego, Calif. 92121-3752.
Rall, T. W. & Sutherland, E. W. (1958) J. Biol. Chem. 232, 1065-1076.
Soderling, S. H. & Beavo, J. A., (2000) Curr. Opin. Cell Biol. 12, 174-179.
Smith, G. D. et al. (1998) Acta Crystallographica Section D-Biological Crystallography. 54 (Pt 5):799-804
Terwilliger, T. C. & Berendzen, J. (1997) Acta Crystallogr. D 53, 571-579 Crystallogr. D 54, 799-804.
Thomas et al. (1990) J. Biol. Chem. 265, 14971-14978.
Xu, R. X. et al. (2000) Science 288, 1822-1825.
J.Navaza, (1994) Acta Cryst. A50, 157-163.

Abbreviations

| | |
|---|---|
| cAMP | cyclic adenosine monophosphate |
| cGMP | cyclic guanosine monophosphate |
| PDE | phosphodiesterase |
| PGK | protein kinase G |
| MAD | multi-wavelength anomalous dispersion |
| PCR | polymerase chain reaction |
| 2YT | 16 g tryptone, 10 g yeast extract, 5 g NaCl per litre |
| | solution |
| Tris | tris[hydroxymethyl]amino-methane |
| E-64 | epoxysuccinyl-l-leucylamido-(4-guanidino) butane |
| DTT | DL-dithiothreitol |
| β-ME | β-mercaptoethanol |
| IPTG | β-D-isopropyl-thiogalactopyranoside |
| EDTA | ethylenediamine tetraacetic acid |
| Bis-Tris | bis[2-hydroxyethyl]imino-tris[hydroxymethyl]methane |
| PEG | polyethylene glycol |
| PEG2KMME | polyethylene glycol 2000 monomethyl ether |
| rmsd | root mean square deviation |
| rpm | revolutions per minute |
| Moi | multiplicity of infection |
| Sildenafil | 5-[2-ethoxy-5-(4-methyl-1-piperazinylsulphonyl)phenyl]- |
| | 1-methyl-3-n-propyl-1,6-dihydro-7H-pyrazolo[4,3- |
| | d]pyrimidin-7-one, which is also known as 1-[[3-(6,7- |
| | dihydro-1-methyl-7-oxo-3-propyl-1H-pyrazolo[4,3- |
| | d]pyrimidin-5-yl)-4-ethoxyphenyl]sulphonyl]-4- |
| | methylpiperazine |
| | (see EP-A-0463756) |
| UK-092,480 | see “Sildenafil” |
| UK-088,800 | 5-(2-ethoxyphenyl)-1-methyl-3-propyl-1,6-dihydro-7H- |
| | pyrazolo[4,3-d]pyrimidin-7-one. |

List of Sequences

| | |
|---|---|
| Wild-type PDE5 “loop region” | |
| HRGVNNSYIQRSEHPLAQLYCHSIME | SEQ ID NO: 1 |
| Wild-type PDE5 catalytic domain | |
| EEETRELQSLAAAVVPSAQTLKITDFSFSDFELSDLE | SEQ ID NO: 2 |
| TALCTIRMFTDLNLVQNFQMKHEVLCRWILSVKKNYR | |
| KNVAYHNWRHAFNTAQCMFAALKAGKIQNKLTDLEIL | |
| ALLIAALSHDLDHRGVNNSYIQRSEKPLAQLYCHSIM | |
| EHHHFDQCLMILNSPGNQILSGLSIEEYKTTLKIIKQ | |
| AILATDLALYIKRRGEFFELIRKNQFNLEDPHQKELF | |
| LAMLMTACDLSAITKPWPIQQRIAELVATEFFDQGDR | |
| ERKELNIEPTDLMNREKLKNKIPSMQVGFDAICLQLY | |
| EALTHVSEDCFPLLDGCRKNRQKWQALAEQQEKMLIN | |
| GESGQAKRN | |
| Full length wild-type PDE5 sequence | |
| ERAGPSFGQQRQQQQPQQQKQQQRDQDSVEAWLDDHW | SEQ ID NO: 3 |
| DFTFSYFVRLKATREMVNAWFAERVHTIPVCKEGIRG | |
| HTESCSCPLQQSPRADNSVPGTPTRKISASEFDRPLR | |
| PIVVKDSEGTVSFLSDSEKKEQMPLTPPRFDHDEGDQ | |
| CSRLLELVKDISSHLDVTALCHKIFLHIHGLISADRY | |
| SLFLVCEDSSNDKFLISRLFDVAEGSTLEEVSNNCIR | |
| LEWNKGIVGHVAALGEPLNIKDAYEDPRFNAEVDQIT | |
| GYKTQSILCMPIKNHREEVVGVAQAINKKSGNGGTFT | |
| EKDEKDFAAYLAFCGIVLHNAQLYETSLLENKRNQVL | |
| LDLASLIFEEQQSLEVILKKIAATIISFMQVQKCTIF | |
| IVDEDCSDSFSSVFHMECEELEKSSDTLTREHDANKI | |
| NYMYAQYVKNTMEPLNIPDVSKDKRFPWTTENTGNVN | |
| QQCIRSLLCTPIKNGKKNKVIGVCQLVNKMEENTGKV | |
| KPFNRNDEQFLEAFVIFCGLGIQNTQMYEAVERAMAK | |
| QMVTLEVLSYHASAAEEETRELQSLAAAVVPSAQTLK | |
| ITDFSFSDFELSDLETALCTIRMFTDLNLVQNFQMKH | |
| EVLCRWILSVKKNYRKNVAYHNWRHAFNTAQCMFAAL | |
| KAGKIQNKLTDLEILALLIAALSHDLDHRGVNNSYIQ | |
| RSEHPLAQLYCHSIMEHHHFDQCLMILNSPGNQILSG | |
| LSIEEYKTTLKIIKQAILATDLALYIKRRGEFFELIR | |
| KNQFNLEDPHQKELFLAMLMTACDLSAITKPWPIQQR | |
| IAELVATEFFDQGDRERKELNIEPTDLMNREKKNKIP | |
| SMQVGFIDAICLQLYEALTHVSEDCFPLLDGCRKNRQ | |
| KWQALAEQQEKMLINGESGQAKRN | |
| Wild-type PDE4 “loop region” | |
| HPGVSNQFLINTNSELALMYNDESVLE | SEQ ID NO: 4 |
| Loop-swapped PDE5 catalytic domain = | |
| PDE5* | |
| EEETRELQSLAAAVVPSAQTLKITDFSFSDFELSDLE | SEQ ID NO: 5 |
| TALCTIRMFTDLNLVQNFQMKHEVLCRWILSVKKNYR | |
| KNVAYHNWRHAFNTAQCMFAALKAGKIQNKLTDLEIL | |
| ALLIAALSHDLDLHPGVSNQFLINTNSELALMYNDES | |
| VLEHHHDQCLMILNSPGNQILSGLSIEEYKTTLKIIK | |
| QAILATDLALYIKRRGEFFELIRKNQFNLEDPHQKEL | |
| FLAMLMTACDLSAITKPWPIQQRIAELVATEFFDQGD | |
| RERKELNIEPTDLMNREKKNKIPSMQVGFIDAICLQL | |
| YEALTHVSEDCFPLLDGCRKNRQKWQALAE | |
| Full length PDE5 sequence comprising | |
| PDE5* | |
| ERAGPSFGQQRQQQQPQQQKQQQRDQDSVEAWLDDHW | SEQ ID NO: 6 |
| DFTFSYFVRKATREMVNAWFAERVHTIPVCKEGIRGH | |
| TESCSCPLQQSPRADNSVPGTPTRKISASEFDRPLRP | |
| IVVKDSEGTVSFLSDSEKKEQMPLTPPRFDHDEGDQC | |
| SRLLELVKDISSHLDVTALCHKIFLHIHGLISADRYS | |
| LFLVCEDSSNDKFLISRLFDVAEGSTLEEVSNNCIRL | |
| EWNKGIVGHVAALGEPLNIKDAYEDPRFNAEVDQITG | |
| YKTQSILCMPIKNHREEVVGVAQAINKKSGNGGTFTE | |
| KDEKDFAAYLAFCGIVLHNAQLYETSLLENKRNQVLL | |
| DLASLIFEEQQSLEVILKKIAATIISFMQVQKCTIFI | |
| VDEDCSDSFSSVFHMECEELEKSSDTLTREHDANKIN | |
| YMYAQYVKNTMEPLNIPDVSKDKRFPWTTENTGNVNQ | |
| QCIRSLLCTPIKNGKKNKVIGVCQLVNKMEENTGKVK | |
| PFNRNDEQFLEAFVIFCGLGIQNTQMYEAVERAMAKQ | |
| MVTLEVLSYHASAA | |
| EEETRELQSLAAAVVPSAQTLKITDFSFSDFELSDLE | |
| TALCTIRMFTDLNLVQNFQMKHEVLCRWILSVKKNYR | |
| KNVAYHNWRHAFNTAQCMFAALKAGKIQNKLTDLEIL | |
| ALLIAALSHDLDHPGVSNQFLNTNSELALMYNDESVL | |
| EHHHFDQCLMILNSPGNQILSGLSIEEYKTTLKIIKQ | |
| AILATDLALYIKRRGEFFELIRKNQFNLEDPHQKELF | |
| LAMLMTACDLSAITKPWPIQQRIAELVATEFFDQGDR | |
| ERKELNIEPTDLMNREKKNKIPSMQVGFIDAICLQLY | |
| EALTHVSEDCFPLLDGCRKNRQKWQALAE | |
| CGTGAATTCATGGAGGAAGAAACAAGAGAGCTAC | SEQ ID NO: 7 |
| CGTTCTAGACTATCAGTTCCGCTTGGCCTGGCCGCTT | SEQ ID NO: 8 |
| TCCCC | |
| CGTGAATTCATGCATCATCATCATCATCATCTTCTGG | SEQ ID NO: 9 |
| TTCCGCGTGGATCTGCGCCCGAGGAAGAAACAAGAGA | |
| GCTAC | |
| CAAAGAAAGTTCTGAATTTGTGTTGATGAGAAACTGA | SEQ ID NO: 10 |
| TTGGAGACTCCAGGATGATCCAAATCGTGGCTTAG | |
| ATCAACACAAATTCAGAACTTGCTTTGATGTATAATG | SEQ ID NO: 11 |
| ATGAATCTGTGTTGGAACACCATCATTTTGACCAG | |
| CGTTCTAGACTATCATTCTGCAAGGGCCTGCCATTTC | SEQ ID NO: 12 |
| TG | |
| CGTCATATGGAGGAAGAAACAAGAGAGCTAC | SEQ ID NO: 13 |
| CGTCTCGAGCTATCATTCTGCAAGGGCCTGCCATTTC | SEQ ID NO: 14 |
| TG | |

1. A crystal of a PDE5 protein comprising SEQ ID NO: 4 or a sequence having 95% identity thereto into which a ligand is capable of being soaked such that the ligand is bound to the active site of the protein.

2. A crystal of claim 1 wherein said protein comprises SEQ ID NO:4.

3. A crystal of claim 1 wherein said ligand is a compound of the formula represented in FIG. 4.

4. A crystal of a PDE5 protein comprising SEQ ID NO: 5 or a sequence having 95% identity thereto into which a ligand is capable of being soaked such that the ligand is bound to the active site of the protein.

5. A crystal of claim 4 wherein said protein comprises SEQ ID NO: 5.

6. A crystal of claim 4 wherein said ligand is a compound of the Formula represented in FIG. 4.

7. A crystal according to one of claim 1 or claim 4 which is grown in a buffer in the pH range of 6.5 to 8.0.

8. A crystal according to one of claim 1 or claim 4 which is grown in the presence of an alcohol.

9. A crystal according to one of claim 1 or claim 4 which is grown in a solution containing HEPES buffer, polyethylene glycol 4000 and iso-propanol.

10. A PDE5 protein crystal comprising one or more of the following characteristics:
(a) a space group C2;
(b) unit cell dimensions a˜56 Å±1%, b˜77 Å±1%, c˜81 Å±1%, α=γ=90°, β=103°±1%;
(c) one molecule per asymmetric unit;
(d) a protein of a molecular weight of approximately 40 kDa±2 kDa;
(e) a calculated solvent content of approximately 44±5%; and/or
(f) a monoclinic crystal system.

11. A crystal of a PDE5 protein including SEQ ID NO:4 or SEQ ID NO:5 or a sequence having 95% identity to SEQ ID NO:4 or SEQ ID NO:5 wherein the protein has an active site within the third sub-domain of the protein which is bounded by Helices 15 (H15 813-824) and 14 (H14 772-797), the C-terminus of Helix 13 (H13 749-765), and the C-terminus of Helix 11 (H11 706-721) along with the loop region between Helices 11 and 12a (H12a 725-731) as shown in FIG. 2.

12. A crystal as recited in claim 11 wherein said crystal is soakable.

13. A crystal of claim 11 wherein the protein has an active site within the third sub-domain of the protein and comprises Leu 765, Ala 767 and Ile 768 and one or more of Phe 820, Val 782, Phe 786, Tyr 612, Leu 804, Ala 779, Ala 783, Ile 813, Met 816 and Gln 817 or functional substituents thereof.

14. A crystal according to one of claim 1 or claim 4, wherein the protein has a three-dimensional structure characterized by the atomic co-ordinates set out in Table 4 or a derivative set as expressed in any reference frame thereof.

15. A crystal according to one of claim 1 or claim 4, wherein a PDE5 ligand has been soaked in.

16. A crystal according to claim 14, wherein the protein has a three-dimensional structure characterized by the atomic co-ordinates set out in Table 5 or a derivative set as expressed in any reference frame thereof.

17. A heavy atom derivative of a crystal according to one of claim 1 or claim 4.

18. A method of using the atomic co-ordinates determined from a crystal according to one of claim 1 or claim 4 for deriving a three-dimensional structure of a PDE5 or a mutant, derivative, variant, analogue, homologue, sub-domain or fragment thereof.

19. A method according to claim 18, wherein the sub-domain is the catalytic domain.

20. A method of identifying a compound which is a ligand of PDE5 comprising the following steps:
(a) providing a PDE5 crystal including SEQ ID NO: 4 or SEQ ID NO:5 or a sequence having 95% identity to SEQ ID NO:4 or SEQ ID NO:5,
(b) contacting the crystal with the compound under conditions conducive to soaking the compound into the crystal,
(c) determining, by X-ray diffraction and structure solution, whether or not the compound is bound to the active site of the protein in the resultant soaked crystal,
(d) optionally assaying the ligand to determine whether it is an inhibitor of PDE5.

21. A method according to claim 20 wherein the compound is based on the structural template as shown in FIG. 4.

22. A method of designing a compound capable of associating with PDE5, comprising the following steps:
(a) computationally creating a three-dimensional representation of the structure of the protein present in a PDE5 crystal including SEQ ID NO: 4 or SEQ ID NO: 5 or a sequence having 95% identity to SEQ ID NO:4 or SEQ ID NO:5, or a fragment or subdomain thereof,
(b) computationally creating a three-dimensional representation of the structure of a compound,
(c) co-displaying the three-dimensional representation of the compound structure with the three-dimensional representation of the protein structure,
(d) assessing whether the three-dimensional representation of the compound structure fits the three-dimensional representation of an active site of the protein structure,
(e) optionally making assaying the ligand to determine whether it is an inhibitor of PDE5.

23. A method of selecting a PDE5 ligand from a group of potential PDE5 ligands, comprising the following steps:
(a) computationally creating a three-dimensional representation of the structure of the protein present in a PDE5 crystal including SEQ ID NO:4 or SEQ ID NO:5 or a sequence having 95% identity to SEQ ID NO:4 or SEQ ID NO:5, or a fragment or subdomain thereof,
(b) computationally creating a three-dimensional representation of the structure of the ligand,
(c) co-displaying the three-dimensional representation of the ligand structure with the three-dimensional representation of the protein structure,
(d) assessing whether the three-dimensional representation of the ligand structure fits the three-dimensional representation of an active site of the protein structure,
(e) optionally making and assaying the ligand to determine whether it is an inhibitor of PDE5.

24. A method of using of the three-dimensional structure as derivable according to claim 23 to design site-directed mutants of PDE5.

25. A method of selecting a PDE5 ligand from a group of potential PDE5 ligands, comprising the following steps:
(a) computationally creating a three-dimensional representation of the structure of the protein present in the PDE5 crystal, including SEQ ID NO:4 or SEQ ID NO:5 or a sequence having 95% identity to SEQ ID NO:4 or SEQ ID NO:5, or a fragment or subdomain thereof,
(b) computationally creating a three-dimensional representation of the structure of the ligand,
(c) co-displaying the three-dimensional representation of the ligand with the three-dimensional representation of the protein structure,
(d) assessing whether the three-dimensional representation of the ligand fits the three-dimensional representation of an active site of the protein structure,
(e) optionally making and assaying the ligand to determine whether it is an inhibitor of PDE5.

26. A method of soaking a ligand into a crystal according to one of claims 1 or 4 comprising the following steps:
(a) incubating the crystal in an aqueous stabilising solution comprising buffer and polyethylene glycol;
(b) combining the compound with the stabilising solution; and
(c) optionally adding a cryo-protectant to the stabilising solution.


SEQUENCE LISTING (14 sequences)

SEQ ID NO: 1 - protein, 26 aa, Homo sapiens
HRGVNNSYIQRSEHPLAQLYCHSIME

SEQ ID NO: 2 - protein, 342 aa, Homo sapiens
EEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 3 - protein, 874 aa, Homo sapiens
ERAGPSFGQQRQQQQPQQQKQQQRDQDSVEAWLDDHWDFTFSYFVRKATREMVNAWFAERVHTIPVCKEGIRGHTESCSCPLQQSPRADNSVPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 4 - protein, 27 aa, Homo sapiens
HPGVSNQFLINTNSELALMYNDESVLE

SEQ ID NO: 5 - protein, 326 aa, Homo sapiens
EEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHPGVSNQFLINTNSELALMYNDESVLEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAE

SEQ ID NO: 6 - protein, 858 aa, Homo sapiens
ERAGPSFGQQRQQQQPQQQKQQQRDQDSVEAWLDDHWDFTFSYFVRKATREMVNAWFAERVHTIPVCKEGIRGHTESCSCPLQQSPRADNSVPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHPGVSNQFLINTNSELALMYNDESVLEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAE

SEQ ID NO: 7 - DNA, 34 bp, Homo sapiens
cgtgaattcatggaggaagaaacaagagagctac

SEQ ID NO: 8 - DNA, 42 bp, Homo sapiens
cgttctagactatcagttccgcttggcctggccgctttcccc

SEQ ID NO: 9 - DNA, 79 bp, Homo sapiens
cgtgaattcatgcatcatcatcatcatcatcttctggttccgcgtggatctgcgcccgaggaagaaacaagagagctac

SEQ ID NO: 10 - DNA, 72 bp, Homo sapiens
caaagaaagttctgaatttgtgttgatgagaaactgattggagactccaggatgatccaaatcgtggcttag

SEQ ID NO: 11 - DNA, 72 bp, Homo sapiens
atcaacacaaattcagaacttgctttgatgtataatgatgaatctgtgttggaacaccatcattttgaccag

SEQ ID NO: 12 - DNA, 39 bp, Homo sapiens
cgttctagactatcattctgcaagggcctgccatttctg

SEQ ID NO: 13 - DNA, 31 bp, Homo sapiens
cgtcatatggaggaagaaacaagagagctac

SEQ ID NO: 14 - DNA, 39 bp, Homo sapiens
cgtctcgagctatcattctgcaagggcctgccatttctg